SEQUENCE LISTING (233 sequences)

SEQ ID NO: 1 - DNA, 2001 bp, Homo sapiens
aaattaaaaaaaaaatttttttttttgagttggggtcttgctttgtcacccagcctggagtgcagtggcacgaacatggctcactgcagcctcgacttcccaggctaaggcgatccttcctacctcagcctcccaagtagctgggactgtggacgcacaccactacacccggctatttttttttaaaattttttatttttttggtatttttgtagcaatggggtttcaccatgttgcccaggctggtcacaaacttctgggcttggatgatcctcctgtcttggcctcccagtgttaggattacaggtgcgagccaccgcgtctggctgatgtcttctcatgatgagattccggttgtgccttcctggctggaatgccacatagatgatgctgtgacctgcgaagaccttcacatctggaggcacacgacaccctcctgcctctcacaggtgatttattttaaccccttggtcaagatgtctgattcctctactgtatagttactctttttccttttgttactaataagcaatctatcaagagatgcttcgtgaccgtgctgacaccctcccccctcactgaagtcttccccccagatttagcccccactgatgagttttgcacacaggctgtcttcttgtaggcagccccctctgctacctttgtttttccgggaaggaccttagatcctgggtaggtgctcatcctgcctggcagtcgagtgcaatgctgctgtcctggccaatgcccagccttcctcatttgccccggcttcaaggcattggttcacagtcccgtacgggcagctagaccatctcacagcaagcagcactctgggaggctcatctcatttcctgccctggaagcttcccttcctcactgcagccactgaagctgcaggctagatcaaggggaccacagagttgtccagtccccagggtgggggtgccaggagcccttgtcctccgtagcaggtctcagaggctgacgatggggaaatcgcactggtgaagctgccgtggggtcaggaggaacctgcgataagacgcccaacagttagggcaggtgctttgaggtttggagcctgggaccccacaggaagtgacttcgtgctgagctaatgttgcttcatcctggcgccagtctcaggccaagagctgcaggctggcatggctggggggatgtcagcggagtgccctgagcctgggccaggaggtctgcagggccagtccccagggccaggcaggcagtgtccccctcccatcacgcccacctcctggagcctgcccccgtggagggcctacgtggctgccgccgtcctctgctacatcaacctcctgaattacatgaactggttcatcattgcaggtgaggaggggatggctaccctgggcagtacctgctgctgtgcccaccagccaggggctgcagatccatggtggccttccagccatcacccatctttctgctcctgggcccatctctggtgttctcacctctagaggtggttttcagctgtgctcgaatcggggaggaccaactgatgttcccaatccagcctcaactaagaagggacacaggctgggcatggtggctcccgcttgtaattccagcactttgggaggctgaggcaggaagattgcttgagcccaggagtttgagaccaacctggacaacatagtgagactctgtctctatttatttatttttttttttgagacagagtctcgctctgtcgcccaggctggagtgtagtggcgcaatctcagctcactgcaacctctgtctcccaggttcaagtgattctcctgcctcagcctcccgagtagctgggattacaggcgtgcaccaccacatccggctaatttttgtattttgagtagaggaggagtagggacggggttttaccatgctagtcaggctggtctcgaactcctgacctcaggtgatccacccgcctcggcctcccaaagtgctgggattacaggcatgagccaccgt

SEQ ID NO: 2 - DNA, 2001 bp, Homo sapiens
attgtggcctgtgtgtaagagtatgctcatgtgaagctacattgtgtttgcttgtggacatgaggtgtttgtggctcctggatgctttgtctgttgaatcctgaccccaagtgacctgctgatacagggcaccttggaagtcaattaggccaaaagaccgtgtctgtgcactaatgagaagctttccccaggtgcccctgcccaccttctccacctctcctgtggttcctgcctggcttgggtttggcctttttaacccaaagacaacatgcatccaaatgagattttctctagtgcatacgtctctctctccctcccttctcctctgccttccctgtatctcagaaagaaaaggcctaggaataaggaaaaggaggaggaagaaacatttttttgaggacataccatgcggcaggcctgtgctgggcagtatacacgcattataggcaattgtagacagggcttcccttgtgtcaggcactgttctaagcacttcacccctaaggctgggttaatccacgtatccaccctatgaggtaggtgttgttcttttcccactttcacagagaaggcaagaaatcacacagtgagaaagtggcagagctgggattcaggcctcagcttctcccacacaagcccacagatgggaggatgaacaaggagtcagagtggacctgtggctctcccctgagaggataccaggattcgaggctcagcgccctgggaggagtgaacccctcagtttcctgagcctcatcagaagcagtctggggaggctggggcccagccaatggcagtgcaggtcccagctcctcctcctcccaggctgggccagcacacccggcaggctctgtcctggaaacaggcttcaacgggcttccccgaaaaccttccccgcttctggatatgaaattcaagctgcttgctgagtcctattgccggctgctgggagccaggagagccctgaggagtagtcactcaggtaagggcatctgtgtccgcatgtgccctgccccttggcatacgtggcaggaggacacttctctgctggggagtggtgggatgtggcctggacacatctgccaccctgggtcctgatggtggaggaataaggggctctgggttctttcaggtctccccagccgggcaaaggtaggagcaggggccaggccggaagctcagcaggcagggtagctctgcctgggcctgcaaagccctgccccactcctccccaagtagggatgggctgggtgagagcccctggggctaagggcggggccttcctgggtgttccctgagaagctgcaggagtcctaccttagacaggactgagcatgcaagatggtgattcaaaacagagctccccccaaccccggcccccacctccacccatcccccgtccaagtggcgcaggtggtgtggagacagggtggtgggcaaaggttcccccagccaccctcatccaggatccacacacaaggggaaggacagaatccgtctgcacacaaacaggcaggcagctcattgaaacttgaggaaagggtctgaggcctaaggctgggcagttcccggagggtgggctctagagccagcctgcctgggttcaaatcctagctctgccacttattagctgtgtgacgttgagcaagttaattaatccctctgtgttcctctttcctcatctgtaaattgggaataaaaaatatgccctttcctaaggttgctgtgaaggaataatgtactgattcatacaaagaacttagagcggtgcctggcacggcatgaatgcttcatggatattggctgttatcactactctgctttgcagttgatcatgcagaggctccagagagcctggtgagcttcagcctggaccagcatgggaaggccatcatgagccctttagtggctaggcagacaggttccttggggtcccagggtgcacagttggttgaaaccacagtgcatgggtcttaatggacacacccccatctccaggaa

SEQ ID NO: 3 - DNA, 2001 bp, Homo sapiens
tgggtccagagaggttaagtgacctgttcaaggtcacacatcaatccagttgtagacctggggccagaatccaggattagatctcaccaggctctctgttcccaccacactcttcttccaaatggggcatcagctcctgggtgaggactgtggatctgccacaggaaccccattccaccaaccttcccaagcctctccatcccaatcccacatcgctccccttcacctgcacaaactgctccatggcccactggtccaggcaggtgtagttctccaggaagcgcagcttctccaactgaaactggtccctggactgagcctccgcctgcctctctagcagctggaagatagtggcaccgagcagcaggtagcagacataggccagcagcaggggcagcacccggccaccccagcagctgcagagcccagcactgggcatgctgtggccaggaccctgccagggccgtggggctggctatgggggagaggtgggaaacaggtgaggagtaagcacctaggggcctgtgcccaggctgccgcctgccctgccctcctcctcggcacaggtgtctggaggctggggagactgtttgaacagtgcggctcagcttggctgcactaagtgctatgctgtgagtgcaaacaggccggccacccccacccggcctttgtttccatggacagcagcaagtcagtgtcagggacccttcaaagagacctaccaggttctcctgagtgcttcatgtctctctctctgtctccaagttggatagatgtttctgggctaaatcggataggtgtgctcactctgtccaggccccagtgggctctctcggccgcccccagcgtcttcacccttgaaggctgtaagaccccagcttaggagctgggaagaggtcagaatgggtatgaataaaccttctactcagttgccttggctacaaaatggggcaggataattgaactgcctattgggggaacctcaggatgagtaaactgcgtgacaaatcagggcaaagtaaatagacaggtgattgtctcactcagcctgatggtaagagtatcagggttgattctgctgataagttggattcattggttttataactcttgctaatttgtcttgcatgaagccctctagtgggttaccttcagaggatatgtcggtgaaatatatgtgtgaattaaaaagaaactataagtatatcctgatcctgggatggtaagaggggctgtctctgagtggatggaattgagagtgctttcctttctctttgcctgtgtgtgtgtgtgtacacatgcttgtttatgtacatgtataacagctttatacagatataattcacatgctgtatcgtccacacatttaaagtgtgcaattcagtgggttttagtatattcacagggtcttcaagatccagccctgttgcaacatgtaacagtacttcttccttctttctggctgagtaatatttcattgtgtggctataacacatttgtttatccatttgatttctagttgatggacatttgggttgtccctgtttccttctattaagaacaaagcagctatgaacctctgtgtatgagttttttttgtggacatgtgtttgcattactcacaggtacatacctaggagtagaatttctggactatctggccactctgtattgaactttttgaggaactgccaggctattttccaaagtggctgaaccattttatattctcactagcccagggcgaggattccaatatctctgctgtgttttctgctgtttcctttaatgagctgtggctgcagacactgtgagttgcctgctgagtatcctttccctcctccccagctagcagaaccccggctttgtaagtgttaaccccaggtaattgagcataattggtctaagtcatagtgctaatcttgctcccctctgtctcagcccctgagagcagtgaagtcactgaagacccataagacagtgggcaagagggccaggctg

SEQ ID NO: 4 - DNA, 2001 bp, Homo sapiens
tgggcgcagcaggccatgggttaccacggccagacccagcagcagcaggggccagcgctgcctgaggttggggcctggctcaccaggggcctcaaggcccccctggcctgtccctggcctcatcggccctcagggtcagaggtccttgtggcctgacttgcagtgtgggctccctagagagtggtgccctggtccctctcgctggcagcagctgtcccattgcaccccagtccatgtggccccatcctcatcctctggtccctgcccccagccaggccccttccagagtccatgaccgttacccctggggttctgtcccagaggcacatctgccaactgccctgctgtggtcaggatccagggctcaactgcctcccacacctgtcagcttggccttgcccctggggccctgggtgctttgtttggggacctcccaccacaggcagagggtcagcaagagctgaggtcacacagggaggtgggatgcgccatggccccccaagaccctcccttggggctttcctttggaggcacctgcgccctggatgggcttgagtcccctcggggctggtcccaagaggtgatgcacctgacttccctccttccccacttccccgagggcgccctgggtctgtggagcatctcactggggtccagagagctgcccagcaggagtcccccagggcttgagctcaggggcctgggctgaggctcctccaaccggtcctagagggaggtgtggttagagagcaggaaacaggtttgggtttagccagaagccactcaacggttcaagtccaggaatcatgagataagaatgaaaatgctgtttaatgcaccatgttcagcacctcactgacctcacatcacacagacacacacacacacaccaaccaccccagcttcctgaaggtataattgacaaaactgtatatatatttaccacctacagtgcgatgctttgccacactgcaaaatgattaaatcaagctgacacacacgtccgtcatctcacatacttatgcttttgtagtgagaacatggaagatctaatcttagcaattttccagtatacaatacagtttttaactgtagactccatgttgtacgatacatctccagagttcattcttatagatttcactcaaaggccagtagcttcctcctgagcggtgagtgcatcatgtcatggcttctgacaggcccccggtcctctcccagcccagcatccccgaccctctcccaacatctcgcgaccctgtgctttggtgttgagggagcagtggcagtcaggagggactgagcacctgtgctgggcggccagggccaggctcttttgctggtgctgccagagacgcctcaggtcacccaagagtctcaggttcctctccttcctccagaggcggggctggaaatgctgctggctctgctcacactccagggctgggttgaggttggtgttgggagataatgcatgtcagtgttgttttcttcatctgtgaaaaagcagtaacatacacacaacctaaaatttatcatcttgaccttttttttttttttgagagtcttgctgtgtcgccaggctggagtgcagtgtggcgtgatctcagctcactgaaacctccgaatccctggttcaagcgattctcctgcctcagcctcctgagtagctgggattacaggcatgcaccaccacgacaagctaatttttgtatttttagtagagatggggttcaccatgttggccaggctggtctcgatctcctaacctcatgatccacccgcctgggcctcccaaagtgctgggatcacaggtgtgagccaccgcgcctggcccatcttgaccatttttaagtacacagttcagtggcattaactacattcacaatgtcgtgcaaccatcaccaccatccatttctagatttttcttttttcagagtcttgctctgttgcccaggctggagtgcagtggcgcaatcttagctcatggcaatctctttc

SEQ ID NO: 5 - DNA, 2001 bp, Homo sapiens
attttttgtatttttagtagagatggggtttcaccatgttagccaagatggtctcgatctcctgacctcatgatccacctgccttggcctcccaaagtgctgggattacaggcgtgagccaccgcacctggcccagaatatttatttagagagtgatgtcaggcctaagtgttgatagtgggtagtagtgggcaagatgcctggccctctctctttcttctcatgagagaaggtggaatgccttccaccaaagaaacgagacctccccatgaccagctgctccactaaccacacatcctccagtgatgcctctgaatggtcccgaggggctgtggtggccgggcagagccaggcaggagccagagtcagcctggggggtgatggagctgaggccatcaccggtctgacagtggaccagtatggcatgctgtataaggtggctgtgccgcctgccaccttctcaccaactggcctcccatctgtggtgaatatgagccccttgcccccggaaaaaaaataatattgcatatgcagttgattaaatggcatactccatggctattaggaatgatgaaaatgggctgggtgcggtggctcacacctgtaatcccagcactttggtaggccaaggtgggcagatcacctgaggtcgggagtttgacaccagcctaatcaacatggagctgtgtttactgagtggggctttcgcaggctggagctaagaatttccagtatgcgtaacagccacagcccaaatatctgccagtgagttgtgtaattccccagagcaggcctgggcagtgtctgggtggggcctgggagccacaggagacgcccaaagccaggcagagcccgggggcgagggggcggcaggcaggtgtagcgctgccctgggcgggcttgcacccccacacccaagtgagcggcctgctcactcctcagctgcaggagccagacgtgtggagtcccagcagaggccaacctgtgtctcttcatctccgtgagaaaggtgcccccgaagtgaaagagatggcctggtggaaagcctgggtaagtgggagctggcaggagatttgggttcctgcatggtgttgggtgctggtcaccagggagtcctgagaaatgtaaagtcgcccttggctgggagtggctcagtctacatttggctgcctcctgggccaagcatggagggtgggaaggggccgagaagaaacagtcccaggcctagagtgcccacggcagggatgggacctgggccgggagacaggctgatgaagaaatctagaaatgcaagggggaagctgaggtttgagaagagtccaggggagggacccttgcaggggtggggccactcaaaggagggggtatcatggttgagccttgtgtgtaaatgtgtatggggtgcacacacgtgtgcatttggggagaatcaggcatactgctcctggcagagggaactgtgtgcagagggtcagagggcggggagcttggctttgagggaagctgctcagcacagctcaagggtcctggtaccggaggagatggacctggggaagcaggcagggccaggtcctgaaaggccttgcctggcaggctgaggagttaggctttaccttaagagaagtgggaaggcactgaagggctttgagcatgagagtaacctgaacctgctgttggggacccatcaatgccttcacctcagggatcaatgatgttcatttgtcaagactggagagggaagaggggaagcaggaggacctagcctgctagagcctggaggaacccggggctcagggcagcccatgcccagcagaactgatgggttctcgggagctttctgtctatggttgagcctgaggccatcgctgtgctaaggagttgaatatccagaaggggacagaggtgacatctcccggctgccggctctgcacacccttttcctttcctcccaggcagcctcaggatgtcagggctgcactttctgttttgcagatgatgaaa

SEQ ID NO: 6 - DNA, 2001 bp, Homo sapiens
aaacacaaaaggaaaaaaaataggcaaaaataaagattatctcatatggaatttcagtaagatacttctattacttggaaaattccaaaggtttttgtcctaggagaaatgccacattcttttttacacagcagattcctactctaaccctccccatacttcctacctccttagttctccttgccaccgaattatcctgacatatgtacttacgcgtttggttatttggggcctccatcacattaaactttatgtttgatgacattatggaatttgttttgcttgctcctgtgtttcttgaacctactacactgcctaatataatatgtgctcaaaatttttgaaagaaaaatataaaccaggtaaacaaatcatttcttacagtgaagtgatggaaatcagtcttaatagaaatcagatatccctcattagtcctagtggtgttgctgccagtctttaaacaacttcctaccaaagattttcttcagagccatcatcacttctttgttcctaagggtgtagattagtggattcagtacaggggtgacggcgctatacatgatggccattatccggtcctgaatcatggaggtggctgaagcaggacgaatatatgtgaagcccacaggtccatagaaaagacataccaccataaaatgggaggcacaagtggacagagccttgtggagtattctgcaggacctgttcttaaacagaaggaagccaattacatagaagcaggagagaagagtcagaaagaaagctcccatggatatgctgcctgtgacaatggaaagaagccattgattgagtaatgtgtcactacaggccaattctaagagcggcttgacatcgtagaagaagtgattgagtttctgagagccacaaaaactcaggtgtgcagtcatgacagaatgcatcagagcgtaaaagaagctgatgagccaggccgcagctgccaacagaatacacacctgggggttcatgatgacagtgtagcgaagaggattgcagatggcaacaaaacggtcaaaggccatgatagccagtaaaatggcctctgtgcttcccaaaaagtggaagaagtgtagctgggtgatacagcctagaaaagatatagccctgcgactgcacacgaggtttacgagcagcttgggcagtgtcactgaagaataagaaatatccagacaagaaaggtttcccagaaaaaaatacataggggagtggagttgtggttccaaaacaaccatcaccaatatagatccatttccaatcaagtttatcaggtaaatgattaagaaaatcccaaagaagaaaggctgcagctcctgaacaccagtcaggccaagtagaagaaactcattcattgtagtgacattctccattgctctgggaagcaaatttaacaataacagaattaatttttctgaatttttaattttacactgtgaggattaaataaagcaagtttactatttggaggaacctaggggtgagcaacagtgtgaagaatagttacgaacagtaaaactaaatttatgtacaacaaaattgaggaagactaatatacctgataaattctgagctgttaaaatggcactagactaataaaagcattatattatgaacaaatcagatatagataaagggcatttgttttcaaaactaggaagtgcaagatttgatggacataatcataacctcatgtatggcattagaattatgtattacaaaaattttaaacatacaaacagggaatagtttttcaacttatgattctaggttctggcaaaaattcagatgtagctcagtggggatttcatcacttcttctaagatacataaaagccacaagagagtaacacatgtcttctgacaacccaaactttcagtgaaagtaagaaacataaacacccacaaaccaaatgttgtatgagtagaggagaaaaaaacagcaaaattagcatcattagtcacaagactgtgtcatagcaatgaggca

SEQ ID NO: 7 - DNA, 2001 bp, Homo sapiens
ccagaggaagtgggggtcttctgaagggtttaagtaggctgggaggacatgatcttaggaagctcactctggtgtcagttgcaggatggatttgagaggagcaagttaggtgtagatgcccatgatgatgccaagatctgggcaacagacaggaaggccctagctcagaagtggctctagggaaggtgaggtgcatagaattgagagatgctcagtagatggcatgagcagtgcttgatgattgtctgggttggtggaggaaggtggacagggaaaagcagaaagctacgatggtgcctcaaggggctgagtgatgtcactcacagagacagaatgtatagagtgaatgttcagactcacaggaagtccaaaactacataccccaacgtgaggtgctgtgggacatccggggtgcagggtccagagagcaggtaggtagagtttagaagagggctgggtccacaatgcagccttggatgttctcaatgtaagagttgtgggagatgaagccttgtgagtggatgggaacacccaggtgcatttcaggtgaagcaaggggacaagaggctgaggacacagacaagcaaatcctagatcttccatcagtccctagaaggcacgatgtgtgcccctcccagcacacagcctgagccctagcacagagctggccgcagagagggcagcagtgaatgtgtcctcggtggttcctccagatggggcctttgtccgcagtgcacttgtctctgcctgggttgctatagtaacccacagatgcagagagacttggcctccgtgttgccatggaaaccagcaattgggtgtccctgtgtggcatggccactgagaccttgaggattcagtctggtcctgaagggtttggggggagactgcgaccagaagatgtttccatgtcctaattaatgggtgatggtggttgttagtctgactgttgccacggtgatgaagggagacatccaagtgctggtttcagtactgaggcgaatacagggaatttcaacaggctccaggtcttactatgcagcctgaagtgggaccatcccttaaacccactccatcctgtggccacgatgggggccaggacacctttgccttctttctgggtttctttctttgccgagacagggattttgttcccaggaggcactccctggcccatgggatctcagcattcaaagcagcacaggaaacctgggcccctgaaacggggccaccgaagagatcggtaagctttcaacatcctgcctttagcccatgggcccaaccattgcgtcagctccacccacccaccctcactgacctcaaccctttacagtcccaaggcctccccttcctgatggtgctgctcccctatcaccacgtggagccccacaggtatatccccgcaaacccctcttgctccagacaggtgatcagggaggcttcattactccaggagctgcccaggacctcagggaggtcccccaccatgggagtaagggcctctcccccagcctccagggctccggcagctccacactccggccctctgcctcttctggagggctcctccttccagcttctgcctgtctccccaccccccagtgcttcattcccatggctcaggtcatcactgccctcatctggactgcctgcctgcctgcctgccatccggggcccacagagtgagcactctatggaagtccctctaccctgatccatcacgtaactcacctactgaggcagctgagaccagggcactgaaggaggccttgagtgaccaggtgtggccaggcagcaggcagggatgatgaggtgaggctgcaaggatgtggagagtcaccccttcttttcctcctcaggagaacaaccaggaggaagtactggaaggaaggaagggagatcgctcggtgagctggaggggaggagggcgtgccggccctgggaaagggccaaccacaggagactgtgagggaggtgggcggaagacgtgggtggctcagg

SEQ ID NO: 8 - DNA, 2001 bp, Homo sapiens
agcttgagtaaattccaattaattcttcccaactcattctaaagccacagaaaaacctcactgaaaaggacttctgtctacgttaaagtgcttttacccaataccttgtgaagctccctttcctgggggacacagttcgtggtctctggcttttggcaggtggtccactgcatttccacattcaggtgatactccaggtggtcagtgacctgtgggcgccaggcgtgggggaggcacagcttgtccccttctcccctgtctgcggtttctgtgcctgggggctccgctaccccacgtccaagggcaaggagctggtcctaggcggattagcgggctcctctctcctctatttgactcatgattgtttttgtcattccaagagaattaaccagaaaccatcagaaacatgaactttccccctgggcaaatctatcccttgggtaatgcaagtgacgctagtgattctggataggggtctgtaaactttttccgtaaagggctacatagtaaacatcttacgttttattggccatgtggtctctgtaactactcagttcggcatgagtgtggcagcagcaagcagtggcagacaaagcacaggcatgctcatggctaggtcacaagaacacgacttacaaaagtgggcggcaggcagttttggcccttgggatgtaatgtgctgaggatccctggtcctgggttttgtttaagtgccttaatttcatcaccctagtgaatttatgcttctcttaaagtgctcacatcaagctaagcaaggttgaatttcccttgagtgggactattcctgacaccagtggccacccgatgtgggcgtatcaggcctggggagaggcgggaagtcatacactcacctgcctctggactttaaggactcggaagatcctgaagtggtacttgtcatcactttccttgttatactggtcggtgatccactgcaagctgtccttcgcatagttttctactgccatcacttcatggacgcttagaaaggttttcttccttgcttggtaggggagggccatcagagtcaatagaatggccaacaggagctgtagggcctgccagggctcagccatcatccttcagctgcagaggaacaggaagagtgttctctgtactcctcagggacaagtcgaatcacactgaccctacctgccctggcaggatttaagagcagctggtggtggacacccacagctctcctctcctcctcctcccccttcatcagcagctgaactcgaggggagagcagcaagagactgtgcaatggtttttgcataagttcatgccacacacacacaggataacacacacattttctcttttgaggggctgctccctcagcaacacacaggcttctgtggacccagcattgtcttggtgggagccaggcgagggcccctgggagtcctggaggatggtagctgctggagacacggggagattggatgccaggactggcgcattcataggtgagcagtggggggtgggcactgtggggaggggccgcctgcaccggaagccactctcccccagcctggttcctggagacgagttctggaaaatcccctcccttagaggacaaaggagcagaggcaccctacagaaaggtgtgaagggagggagagtcaactctgacaccgacgattccccagtaccgacaagtcctagagtggcctccgctggacttgcatgttttggaggaggaggcagtggcgaggaagcttcaggtgtgagacacagggagctttctggttggacattctggtctcctaaaggtcccatcctggggacctgatgggagtccccagacttggctgccagttagagaaggaagaagccactggtgggtggaggctggagcttgtgtcccacggcacacagaatgcaggcttagtgcggttatgttcgattcctagaaggcctgttatggtgtcttttgggaagatgcgatataaaaatgtgcagtgatggctcctg

SEQ ID NO: 9 - DNA, 2001 bp, Homo sapiens
agggtctcctcctattctaaagctaatattcagttgtatatatacatataaaccacagtccctttatccattcatccactgatggacacctaggctgtttccataattctttagtaaatcacagaaccctcttgttttctcttctaacttagcaaacttcatttgattgagtcacaacaaaatatttatcacaagtaaacaagaaagatggaaaatttctgatcaatgatgaccatccatgttgctgatgatggaacaaaaagccagattaaacaagtaagacagataaaatcgttgattagatccattgcttcaactagaaagggaacataggtgaaacatctactctgtactcttgtatgtggggttttaaaaacaaaccttaataccaaaatgaaagacaagatcagcaaatattaggtgagaagtttaaaaaattattggtattttgcatataggaaaataatttaaaataaagtccctttacttcattgtacatttaattgaatttggaggttttttatagtaagaaatgaaagttcgattgagaagagatgccagctttcactcaccatttctgacagtctcccaggaaccttcttcgtacactgcctttggtaagctcctttctcactggctttattggcttgtgtctttcatttgtaggaacagttaactgacaaagctgcagaggcaggctgagaggtggctccaagagaaccctggccaagccatttatggtcaggggagtagccaagccctcagtccatccctcgggaaagatgggctgctgctgcatggccgacgtgtatggcctggttcacccaccctctttgccttgattttctgtccttacttttcaggctgctttcttggctatacctccatctcagcaaagaagccacagctggcctgagagcttgggggcttctgcagcctgcagccctttggaaacaatgggttacatgaggctgaggctggccactccatccccagctgacgggagtccacattaaccatgttactgataatgatggagtcattgtgttacatggacaacagcatggacattcgggatgtctggaggaggggtgggggcccattctgttgcaggagtcacactgctgggggctccagctgctgcaaggtgctgcactcagtggccaatgagctgcctggtgtgagttcagcctcaccccctctctgtttttgtattttgggtgggaagctctctagacctggaggaagccatggacttgggctgctttataacttacaatttccctgttagggatttgcctctttaaaatggtgaatgagttgctaataaaacaacagaattaaaccaataatttggccattgaaatgaaaaccactgtgagaggagcagataggagtgtaaggagagagcgactcaagtgacagaggaggccgttgtttagatgactctgggagatggcactgaagctgtgcgtgaaagatgagaagagggcagacatgtgaagggttgagggaggagagtcccaggcagaagccacagcaaatgctaagccctgtaagcttggtgcactcccagagcaggcaggaggccggttggctggaacaacataaaccagggagtcacccgggaaggggctaaggacggagtgagtttgagcccttttgatgtgacatgggagggcatggtaagattttcatccttagatgaaaccttttccaagggtttttcagtgggagagatgcatgatgtaattgctggtgacgaggattgctagggcttctctgtggagccaagattacaggggaagagagtggaagcaggtggctggctagtggagtccaggtgagcgatgaaggtggctgctgtaggaagacagcagtgaatgattgtgctctctactgtggaggtggagccactgggattaaatgatgaaaaaaatatggggcggggaagggcgaggtgagggaaagagaagaataaagccaactcctgggtttgag

SEQ ID NO: 10 - DNA, 2001 bp, Homo sapiens
tatttaaagatcatgtcaaggggaggataatggggctcagagaggcaaaatttgatggtgtttattaagcagagagaaacacagaagctggagccgtctagtccttagagagtgtgctccaaggtgggaccagggagggagcaaggccttggctgggcctgggcaaagctaccagaggaccaaccggcttcctctcctctgattccacagcctactgctgctggtgaatttctgatcaagaattctgctccccacaaagaactgcctagagggtgggaccaagccacccctcacacttgagcatccagtccactccatcgtgatgatgatgatgatcatagtattggtgactacagctaaccctcattaagctctcactatgtgccaggctccgttccaagctctttacaaataccaactccttcaaactccccaaccaccctcagaagtagcgtttctaccccgattttatagatgagaaaattgaggcattgttaaataactctccaagtttatacagcttgtaagtggaaaagccaagattcaaactcaggccatctgactccagaacctgcacatcagactgccccgcacttaactgttgttggtaacagttagttgtccatctgcttagcatgtccatcccttctggggttggctggggcaagtcagggcccaactcggcacagccagctccggagcactaacctaatgggggttttgcaagactcgggactgtgtgcttcacctggaaagtgccaggtgtgtggataagccatgtgagcagctggcttcctgggcaatgcccctccccacttcagtgtcccaggcccctttagggcccagccctccaggcactgtgagctgggaatgaggtgaaggaggactaggcttaccctctgtgtgcctccattttctcaccagtaaactgtgtgtgttggatcttacgatcttctaaaataactccagctccaaatgaaggccagtggacccatgaacacgctggctcttgcccagtgtgctgagaagagggcaggagggaggagtagggtgggcacggcagggcacagggtgcagggcaggtgggttcagtttgatttcaaagccgactgcccgccgccccaggtgctctggtgtacccaataggggctgcattggccaggcggcctaggctctgcgggtggagtgaagatttctctacctcccagtttctcccttttcagctctggtaggagagaggaagagtgaagaggaagtcaaaagttctgttcatctggaagggctgggagttgcttcctgggatggggagctccgaagccaattcagaagacggccccggggtctggcggggaggcccaccgctgcccagagggctcggccggccgggagggggcgctgtgtcctccccacgccctgcccgcgggctcctagcagcgttcgccgcgctgcaacgcaatgcgcgcccggctttcccctgggcagcgttcagtgcccgcttgtcctgcagggcaccgccgtctccctttcatcctggggctcccaggtccaaagggttcccagatccagcctcttgcctgaaccaaagtggccaggcactcagtttgtaagatgaacaaatgaaggaaagaaggaacgaatgaataagggctcatcttgcagagcgttcgcgtctcagcgcgcaccaggcacatcagtgccaaaggtgctgggggtcgggcccagcgcccggcgcggctggaatgggggcggcgaggagctcctcctagccagccgcagggcgggaactgcccaaacgcaggtcgcagcctcactgggttccgcggcggctccgctcagtagggaccgccctgttccctagcccccaatagcctcgccctcataaattcacctgagcaccctggcccaagtcaccccagatccctgggacttcccaggccccgacctcggccgggcgggaggctgatcctttaccgcggcccccttcccccattcccggcttct

SEQ ID NO: 11 - DNA, 2001 bp, Homo sapiens
aatgaggttttcaacaagcgtcttggacccttagaagtttcaagtgacagcccttattagtggcaccccttaggggctccctcaaactcaatgccaagactagcctgactggagggaacttagacaatggagtgggcatttgatgttccagcatcttgagtgggtgccattattctgtgacacctggatcctggggttccataagctggggttctaggatgtccattcttagttgagtctcatttcctgtcatatcgacgtcaaaggcccaagaccttcctcctccccttgagcaaacaagccacgccctgcaccaaagtccatctcaccttccagctaccctaggtaattttcctggtaattcagtgtttctaggaaagcatgagtctttccaccccccgccctgagatttactgacatatgaacacatatgattggcatgatataagaaccactcatctgctggccatctctccaggtctggagccagagaagatgatctgaaattgtagcaggagaaattgaggtaggatactaagaaagcttttcaggagtggggctaggcaagaggtgcagcatgagggaataagagtgaatcctcagtatctaagggaggtggcaggtggcgggggacttctttctttgggtcatctttggtggtgatttgacaggaaggaacaaagtggcttcaaacattatacaagtctctagtctgttctgtgtcctgttttctttctcattctttcagtgtggaatctatatgaccctgggagggatgttggttggaagaatgaccagctgatggagatgctgctgtaattattggtggtaataatgggcagcagtgagccacccggtgtgacagtgtaggagaaaacagtccaaactcctgccaaactctctctactgatggcaaatcagaggagactcaaattgtaagtttatagtggtctggcttttggccatgacaatgacaccttgcccttttaatttggggcccgtgcaaatattcactgaaagctgtcaagaggaaaacagaattggttattgaatcacttgcttcctctaggtgtatgaaaaataatttcaagtttaacaaacacaaggaaaccgcagggtccatgtcaaagctgatgagctatttctgaaactcgtgcagaattgtggtttgtgtggtctatgtcacggcacccttgagggagagtgggcaattgcctgaacttggaggctgtgtcctgtccccaggctgctccagggctgcctccttccgactgggccttcttatctgggactgttgagggcaacaggccttccgaagaccagtgaagaaggaggccctgcaaacaggaggctgacagggtaggaacgaggccatgatccctttgcagaaggacaaccaggaggagggtgtctgccccatctgccaggagagcctgaaggaggccgtgagcaccaactgcggacatctcttctgtcgagtgtgcctgacacagcatgtggagaaggcctcagcctctggggtcttctgctgccccctctgccggaagccctgttctgaggaggtgctagggacaggctatatctgccccaaccaccagaagagggtgtgcaggttctgtgaggagagcagacttcttctatgtgtggaatgcctggtgtcccctgaacacatgtctcatcatgaactgaccattgaaaatgccctcagccactacaaggtaagcctgggtcaccgcagccaggccctgcctccacctcgctgaggtgctgcatcctacatgttcatcatgcctggcacctcagagtagctcaacaatggacatctctctttgtttcttctgcttcatcctgttttggacccttgtcttgcttttctgtgtatattttgaggctgatgtttccatgcattaatgtgagtctgtctaaaagaggatattgtcagtgtgatgttagagtcccagtctgctcatctgtagaatagagtaattggactaactaatg

SEQ ID NO: 12 - DNA, 2001 bp, Homo sapiens
gacagatctagactcagtttcaaaaaaaaaaaaagactcaggccaaatctccttcctttatgatgcaaactttctcggccccattccacctaatcaggctgctatttcctcccctggctcccagcattacacctctaggtcaagcctttgccacagtaggctataatttatgtatctatccctctacctctctctctctctcttttttttttttttttttttttttttgagacagactctcactctgtcacccaggttggagtgcagtggcgccatctcagctcactgcaacctctgcctcctgggttcaggcaattctcctatctcagcctgccactatgcccagctaatttttgtatttttagtagagacagggttttgctatgttaggccgggctggtcttgaactcctgacctcaggtgatccaccggcctcagcctcccaaagtgttgggattacaggcatgagccaccacacccagccttctgcctctcttgctagaccatgaatttgttgggcgctgggtctctttcctctctttccttggtgcccagcaaagtgctcagcacagaagaggtgcttgatatatgcttattgaagtaatcatcaacaaacatttatcaatcatcaatatatttatttattgaggatttactatgaggtggggaaactgaggcatgaaaaaagagctggttcaaggcaaatctagaatgaggatccaggctcctgccccccagagctagcctgtgcatttgagaaaggaccaataccccccaagataagcatctggaatggggacgagggtgcagcgtggttaccttggatgcccatctcctttcgacaccactggatgaagttagagacattgtccctggcctggaaggtacctggctgggcggccccattgcaggagaccccgacccggggcatgggaatcttctgggcttgggcaggtgcctcagccaggaaggccagggcagcgtcagtgacaacgttggcgtgttggcacagcaccaggcccgtttccagcacctgcaggaagttggctgcgtcgatgtccagcccatagaggtcgcgaagccactcagccaggtcttccttcatggcctccaggtactgctcactcgacttgaaaggccggatactgcacacaggcggccctagggtcctgggcttcctcctgcctcccgcaggctgggacatggctggaccccagcagggcaggaggtgggcacctcccctctcccactgccgcctctttcctcccgctgctgctgggtctcggctgggttcttcctgattctgaggttcccgtgtactcgctgagagcccgggacctccagtgctgctacttgccactggcttcagctgccaggcctggcccagcccctgcccccccacattcctcagtccttgccagagctttccctggtggccctgcagcaaccttgctctcaggctgcccagtccacttggagttcacccctctgaggccagatggaagtgaggacactcctctctttccccagagctggagagatcaagctggctgcctcctaccctctgctggctgcggccacagagcctggggtggctgccggcctccccactctgctttcctcccctctccttggagacagcggctgctacaaacactccttgttaacccttcccagcaggcgaggactgccctgcagaaccaagggagagggaggagaggttggggctggggctggcttctctcacggggctggccaaatgggaactttgggggagtggagggaaagcaggggtaggggttgccataccccttggtgtttagtgagtacaggagaatgggaatgaaacagacatagaaatagggcccaaaactcataagtgtagccttaaatcctgtgtgaccatcatatactacctagcctctctgtgccttagtttctcaactgtaaaatgggggtaataatgtttacctttttggtgtgttgtgagggataaagaaa

SEQ ID NO: 13 - DNA, 2001 bp, Homo sapiens
taagaggaattgctcttgcaggtggtaaaggaaggcaaaaagctgaggatagtaagagtgagaggccgggcacagtggctaggctgtaatcccagcactttgggaggccaaagcaggcagatcatctgaggtcaggagttggagaccagcctggccaaaatagtgaaatcccttctctactaaaaatacaaaaattagccaggcatggtggcatgtgcctgtagtcccagctactcttgaggctgaggcaggaaaatcacttgaactcaggaggtggaggttgaagtgagccgagagcatgccaccgcactccagcctaggcaacagagggagactccgtctccaaaaaaacaaataaataaaaataaaaacaaaaatacaaaaaatagccaggtgtggtggtacaagcctgtagtcccagctactcttgaggctgaggcaggagagttgcttgaacccgggaggcagaggttgcagtaagccaagatgatgccactgcactccagcctgggtgacagagagagactccatctcaaaaaaaaaaaaaaagtgagaaccacactcacaataatattcataatgagaactggcatttatggggcacggactgtgcaccaggcactgtgctgaatgctttctgcgcatgattttgtttgacactcacaacatctgtgtgaggtggggcctgtcactctcccagttagatgatgacgctgaagttcagagagctttgctggcactgcttgagaggaagggccaaccccggctttgaggcctgagtcctcttaaccaccccactgtgttagggacagggttcccctatgattgcagaaggtctgagcagaaaaagccctccgatatcatgtacccaaggctctaggtcttcatggggaaactgaagatcagaaaggggcagtggcctgcacaaagtcacaggttagagtttggaggacacaggtggaacagcacttgccaggcgctgcagtgtgggtggtgacatcgtcagggcccgctgggctcacagggcctccaggcaaccgggggcctttgggtccccagttacacactgtggtcacatcagccaggctgacctgagggtcactgcaggaccagcctctgctgagggctggctcacctgcatggagggggttgctcccaccccaagcatgagtctgacccacgcaccagctcactctcccctcaggaccaccgacttgcccagtcactgaatggagaccacctgctgtggtcaccttgccaggcccttctctgtagacacaggagaccgatcttgaggagccagaggcagagcccagctgggagccggggtttcactggtcagtcggtcagtctctgcagctgcagcctgagatcagccaagcccctcctgaggctcctacctggctcactttgccctttctccacaggggtctgatctctctgctctgcttttccaaactatttctctttctctctctcgctctctggttctctctctctctctctctctctctctctctctctctctcatcagatggctgactggaggcagggtcccagcccaaggatggggttggggtggaggtggcgaacctgggttggtccccactggatgctggtcctcactctcatggcagacggctttctttgaggccaggactgggtgatggtgtcgctacccccgccgcagtctgacgtcacgctgccgggccccaccagactggagggcgagcgccaaggggacctcatgcaggcaccgggcctcccaggctcccctgccccacagagtgtaagtacccggcatctgggcctgggtttaggccaaggcctgcggctgcccaggcaacaagaagaccagtcaccttaccgcccccagggacctcccagggcctgtcttgtcccctccaccagcaaacaagtcctcagggaccccgagatccagaggggtcactcctcatcccttcagtctgagcctgtcccagggctgtgagccccatcaga

SEQ ID NO: 14 - DNA, 2001 bp, Homo sapiens
aggggtaacagcagactagggaggcctggggtcctccacacgcctgtgtggggtgactttggcccccagcaacaaggcaggcctgaggtctgcagagggaggcagcccactcctcagccgggggcgtctatcctcctcctggccccatccctcacctgtgggggaggctaagtgggtgtcagggccctgtcagagggtgagccacccccgggggtgcccatcggccaccagcctcccttcagtagacacccagcccaagtgacagcagtggcatggcactgctgagcacagggagcctcaaggtcagtgcctggctcctggtatcctccgtggtccccaggacacccccacacagaggtcccagaacccccaaagctggcacaagagcccaggaacaccctgcccagcaccctccaggtccctcttttaggatctgggtgagctaaagaagccaaaagaaaggctgggcgcggtggctcacgcttgtaatcctagcactttgggaggccgaggtgggcggattgcctgagctcaggagttggagaccagcctgggcaacacattgaaaccccatctctactaaaatacaaaaaaaattagccaggtgtggtggcgtgcacctgtagtcccagctactcgggagcctgaggcaggggaattgcttgaacccaggaggcagaggttgcagtgagctgagattgcatcactgcactccagcccgggtgacagagtgagactccatctcaaaaaaagaaaaaaaaaaaaaaaaaaaaaaaaaaaagaagccaaaagagggtcttcggcccagcccgcccctccccaacagccctgggtgtggcagggagcctgaccagttccctccagcctgcgtcatggagccctgggcgtggctgcagggtttaaagagccgacccacgtgcccagcagcctcctcagatccgttctctgcgctgccagctcaggtgagccctcgccaaggtgacctcgcaggtgagcaggcgtccactttggggctgctgggtgaagggggtgggtgcccaagaattctgagtaatgaggagcctgttgaggggtctgaggacagatatggctgcagcagccgcttgaaggcctcctgctttgcacccactatcatatggatggtgggttgggggttggaggaaacagaagccagcccttggctaaagaatatatgagctggtggggtatacctctggtcccaactgcttgagatactcaggtgaggagatcacctgagccaagagagttcaaggctgcagtgagccgagatcacgccactgctctccatcctgggtaacagagcaagaccctgtctcaaaaaaaaaaaaaatagtaataataataataataaaataaaaaatgtgacctagagttgagaccctcgtattccaggaactccaggttggtgaaggtagagagaccagtgaaggctgggggaagccagctttggagactgctcctagcccaagaaccgatgttcagaagcagtgcctaacagtctctccctccagtgcctccctcccactgcagaaggaccatgtgtttatttatggatgtatttatttatttattttgagatggagtctccctctgttgcccaggctggagtgcagtgccaggatctcggctcactgcaacctccaccttccaggtttaagtgattctcctgcctcagcttcccaagtagctgggaatacaggcgcctgtcaccacactcagctaatttttgtatttttagtatagatggggttttgccatgttggccaggctgatcttgaactcctgacctcaggtgatccacccgcctcagcctcccaaagtgctgggattataggtgtgagccactgcgcccggcctatggatgtatttattgtaacaggtttttagtgcttactgtgtgctaggcatagttcttaggcttgacaaatagtagttcacgtaattccatcgctataaccttatgaggttgctactattctg

SEQ ID NO: 15 - DNA, 2001 bp, Homo sapiens
cagagaaagatgatatattttaagaaagacaaggaagcagaaatgtttagtgcagaatctgatctaactggctttcaaaggctggtggtcgtggaatgtgagggaggccactggataaactgaggcctcctcaatgtcagtctcctcctggttcccttggaccagaacaagaccagaccccagatctgcagtgggtcctctcacggccctgggccactgcccccttaccttgttgatgaaagatgcgaaacggttgttgaggcacttgatctgctccttctcatccctctttaccctctgcacagtcgggtctatctccagtgccagtgggaccagcaggctctcattgatggtgacaggggtgatgcaggcagaaggcccacaggtggctcccagtcggtacccgaagccaggcagggtacctccacacctggaggctacccggggcctcccaaagcccacgttgcacaggctccgtgagccaaggcagcccagagctcggaggcccctaccacccccgggccggcatggccccttgctcactgcatagtgggtgaccatccggggcatgacagccgagtatgagctgaaactctgactgccacacctggagcctggctggaaagagtggtacgacatggcaggagagagaggcagagaaatgcggccctggaggaaagaaccggggcaggatgatcaagtcaggctcagcttccccccttttatctggtagggggatggaggactgagatgtgtcaaaccccaaatcaccattaaactaggtttatgagcttgggatattggcagagctaagtagcaaggtggataattagggtagcgaagagcaaagagaaggggccaatggtcagtgctggtgggccctataaaaatcctatttgtcctccttccttgattgtgggggtgatgaaaggggccaaccagacagatatgttccatgtatccagtggctcagcctcaaggaaagggagagttgggactacgtcatttcataggaggggaggcgtgccatgaattatacaccctggcttctctgggattagagggcaagtcccatatgcttaaagatgctctggctctttccccaaggctgctcctaggcttttttgcagatcctcaacccaagtgccctctgtgggctcctaggtgagtcaggaagttcccaggaggaaggcttttggggtgggagaatcctccctacaacaggccggaccttaaccctcctgagctctggctcagtgacccactgtcccatgagggtcttgaaggtagacagactccaggcatagacacttttctacagggcccatcagtccacaaaggctgcagggaactcaagaggagaggcctggtgcttgagaagggcagaggaagggacgctctcctcaacacttctggggcccccagaaggcatgatttcagcatcaggtctcggcttccatgtggctgttcctttccccttttgcccaacccagcctgagtgtccctgaggtcaacagagccagctttggcctgaggatgtctctggggagagttgttgcccttgggaattttcaggtggaggtgggattcaggttttctttccagctcaggtcgcagacacagctctttggagatgtctgaagcccccgcctcactctctttcctcctcttttccatcaatttcatcagtgaaacttttgccatggccgaatggcctttcccttccacactgagacttatcaccaggtcaaaatcaaaccaataataataataataacaataataaacatttattgggcatttattgtgtgttgggaccactccgtactctttacatccatggtctcatttaattctctgaccacacaatgagatagttactatgccacagacaggaaaatgggaagcacagcagttaagagcaacatccccaagtcacacagcttgtggctgatcagctgggagtccatcctgttccttttgacttcaccctgccatgct

SEQ ID NO: 16 - DNA, 2001 bp, Homo sapiens
acgtgaatacacacaaacacacaaacagcttggctgctctgggtgaagcatgtgtgtgtttgggggaaatttccttccccacccgtggtcgtccatttgcccatgagcagcttccgaggcaggtccgtggaccccaacgctgtgtaacaccgtgtgaaaaacacaaaggcttgcagccctctcactgcagacgtgggaacacgactctgtttaaaacccttcaaagcccgccttcttgccgtgtgatgctgcctgggccagcagggcaggtcaccacgctgtctcttcaaagcagctcgctcatgcccacagcgctgggcacaagggcagccacgagctgtttgatttctgcgtccccagcagatggcttggtgcacaggggcagggctcgtctgtccctctcactgctgcgtcccaggctatgtctccaccatgtaacttcagacaagttcctcagcgtctctgtgtcctggttccctcatttgtaacatcaggatattgataaggatcatagtaatagggatgtccttatggggcctcatgaaaatgaagagttctctgtgatgtgcctggaaccacagagggcagcaagtgggagccacagaaagtcatctctttatcatagttgtcattggtaactatgacggttatcaagggcaggggtctcccaccttctgcatccaggccttgtcattccttataaaattttctcctttcaaggactttcaatgccctgaaaacggaatgctcctccctgctccccatcagctgctgcagagccctgaaccccttgaacttgccttgcttagttctttcaggggctgccttggatgccacctcacttccctcttcttggctctgtggctgtcagaggggctgggcactgggttacttcatcagttcctgggtgttgatcggcaggaaacagtggtttcctgttcatatcctgcctgagcctgggccgggtggaggtgtcctgcatggagtcttgtgaaatcgcgtggactgcaacacccagcagcgcggtgagtccaccctgaaatccacagtgacccacgcggcatgtgctgggaattgaacgcttggggaaagagctgggctccagcggatcagccccgaccaagtcgttcccacagctcaggaccggacgctacaagcccactttgtagcagtttggcggacggcaaacttttgcaacacagtgcaaaaaccatttaacacctttaagggaattggaaatgggtttttcatgtgtggtggtgctgaaaattgagaataatgacagaattattaaaaacagccttctcggccttagactcttaattgcctctggtctgtaaaactgacaaataagcactgtagaagccacagatgaaatatgtcgtcttgggagccctggagaattgtctctgaattcttttgtttatggattgtagaaactgctttgggggtagttggatgtagaagcagaaggacagagtggggagctgggagcaggaggcctgggtttgtctcctgagtctgtttccactggggcctgaggctcccgtgtgagggaacatgaatgggccaggcacatgggccctgtggggtgacagagctgtgggctccatggtggctctgtgggcagctagcttggggatggctgagataaagacaagtagttttccttcctgcaggacttctcagagcctttagtgtggttataatgatagaaaacagtattaagtccataccctgaaccagaaacaatgctaaaagctttgcacttggcctttttttttctttttcttttatcaacatgtgtgaggacggaagcttaggcttacagatatggactcattgagagtgagtggacgtgtctgatctttaacccagttctgtctgaccaaaaagcctgtgctctaaccactgtgcacccctgcctgtctgtcctgttgagagaactgtagggaaagccttgagattatgtatccagtgctcccctcctcctgattggaa

SEQ ID NO: 17 - DNA, 2001 bp, Homo sapiens
tgaccttgctcaggtccatctccgggtgtcagtttcttcatctacaatgcaagagggttgcccacctctgagaacccttctaaccccaaatctcaccctatgaatctaagaacaccaacccctcgccatcctaagtatcacagagccaggcaagcatgggtgagagctcagaccatccttgttggactaaaaggaaggggcagactgcccatggggggcagccgagagggtcaggcccccataggtcctcagcctgcttcaacctcaaaggggatggggggctgagtggtgccagaggagcagcaggctcgctcggggagagtagggccttaggatagaagggaaatgaactaaacaaccagcttcctcccaaaccagtttcaggccagggctgggaatttcacaaaaaagcagaaggcgctctgtgaacatttcctgccccgccccagcccccttcctggcagcattaccacactgctcacctgtgaagcaatcttccggagacagggccaaagggccaagtgccccagtcaggagctgcctataaatgccgagcctgcacagctctggcaaacactctgtgtggctcctcggctttggtaagtgagctgccagcttccccaggcagaagcctgcctgccgattccttctttccttccctgacccaacttccttccaaatcctcctcctagaagccctccttggttggccctgcctactttaaagcttctttcacattttcttaggtcatgttcccctggggcctcctgccctcaaatgctttgctttttggcactctgtagatattctaaaaaatcattttgtacatgtgtgtgacaggccatctcccagttaagttgcagcctgtgctttctttttattttgcacttcccccactatttctgtgagtgcttagtaggaagtgtcaaagaagcttgacagcattttcttctaagtgtcccaactcttggttttccattacacagacagagtgcaagacgatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaaggtagggctggactctggcaggtctgacccagcctcaccgcagtttgggttgacaagggaggatgggagtatgggctacagcaatcaaggggaagatttgagctcctggagcccagccccaagacgcagcgagtgtcctgttatacagggcaggtgctcacagttacacaggacgacagggtcaagaaattgctcaattgaacacctgctatttgtcgggccctgttctgggcagagggatgtagtggtaaatggggagcccactattccagtggagggagacacacagtaaagttgttggccaataaagagcacagataaagccaaatgccaataagtgcctggaagaaaatgagatagagtgcgctgtgggcaatggggctgggtggggtggaggtgaccagttagggtacatgagaagggcctctttgaggaggtaacatttgagctgagccccgaatgttggggagggaagcccctgaggatgacacttggcacaaagctgaggagaccctaagcctcaggcgggaacttggggtggaagacttgggggcttttctaatcctaagggtctgcggtggaaaatgaatgcataaagagcacatggagagcacctgcacagcactcagggaactgggaggtttttcccccgctccaaaaatgattaggcagttctaagaaaaaggctgagcacttccaacagcctttttgttttcttttcaaatttggggaaagtcgggaaacagaggcctgcattaagaagggtggaacacatgggtctcagtctcagttccagtcccggagccagacatcctggggtaggtccccagccctcccagtgcccctccctc

SEQ ID NO: 18 - DNA, 2001 bp, Homo sapiens
tccccttttgctattatttttaagagccttgctgctctccaggcaaattctcaggttctctgcccgtctgcagcatgtatgcatatgcgtgtgtgtgtgcgtgcgtgtgtgcgcgtgcgtgcgtgggcgtgtgcgcgtgtgtgggcgtgcgcgtgtgcgtgtgcgcgtgtgtgcgtgtgcgcgtacgcgtgtgtgcgtgtatgtgtgtgttgggggaggctgcggtgcctatgactggttgcccccttgaaccccaccagaagccagcccaggcccctctgcgccctcttctgagtctccagggccggttctctcctggcaactccagaaagtgcctcctcacctcaaaagaccaagaaacagggtttctgataaagtgtcccacccagtcccggctggggcagttggttagagaatgaggggacattggggggaagaggagggagaatgaggggatgggaagagaggcgcagagaaaatgagaggtggagagaaagggacagagtgaaagagaaggagaaaggggagaagccgggaagacaatgagaatggaagcgagagatggaagtggaaagaagaactggaagcctagagacagagggagagagggagagagggagggggagagagagagagagagagagagaacgggaatgagcagctgtgatgtggccagatcagttcatagaaaagaaacacagttcagagcagaagggaggaaagaaaataagaaggaaagaaacagagaatgagatgcagagaggagaggagggagagagagaaggaaaccgaggtgggtagaaaaatgtgacgaaagggaaggacagaaaaaaaaatgagagaagtaagacagaataaaggagaaaaagaagaagggaatcatgaggatggtgagaaaagacaggagagaaagccgggagagaggatagaggggcgttctcgctgcgctgcgtgtcagctctggtaagccgtctagcatgtcattagtggggacctgaggcgccagcgggaaagtcccctccccgtgggctggaggctgagggagggcgggccctgggctgggatccacaggcaggtggccgcccctgggtggggccgcccggccaagccctaggggtataagtgggaggccgggcctgctgagcacctgtcaggggcagctccggggtgcaggtgccaggcaggtggctccggccagcccagccccagcatgagctccttcgacctgccggcgccctccccacctcgctgcagcccccagttccccagcatcggccaggagccccccgagatgaacctctactatgagaacttcttccacccacagggcgtgcccagccctcagcggccctccttcgaggggggcggcgagtatggggccacccccaacccctacctctggttcaacgggcccaccatgaccccgccaccctacctgcccggccccaacgccagccccttcctgccccaggcctatggagtgcagaggccgctgctgcccagcgtgtcggggcttggggggagcgacctgggctggctgcccatcccctcgcaggaggagctgatgaagctggtgcggccaccctattcctactcggctctcatcgccatggccatccacggggcacccgacaagcgcctcactctcagccagatctaccagtacgtggccgacaacttccccttctacaacaagagcaaggccggctggcagaactccatccgccacaacctgtcgctcaacgactgcttcaagaaggtgccccgcgacgaggacgacccgggtaaggaggctttgagtgtggggggtgtccccaaggaagactcacttccttcctccccaagaccccattctagaaagttctgactagggctgtgccccccaagactgggggatgctaccaggcaggacagaggggagctcagcagtgaaggagagcatgggcctgggggaagatttgggtttcagccctggctgtgctcctgcccagtcagtgacct

SEQ ID NO: 19 - DNA, 2001 bp, Homo sapiens
gtaagattcaggggctgctatggatgcattacccaggagataagggcaacctcaggtgggccggggccctcacagcaaccttacgagaggggttgtatcatgcccatttgatggaagagaaaactaagattccattagaattgatgtgacgtatcctaaatcacacagctgcagcaggactaggatttgaatgcgggtcaatctgcctccaacactactgccttttcctccatgctggattaggctattggcttctgaaagcacagacaggtgagatgggtcaaacatggcctgaagcacccaggcacaggtgggagatggcaagcagaggcaggttcaaaacgtggggccaggactggtctgaaagatagtcgttaaatagcactgaggagagggagtgagagagatgtgggtcttgggggagtggggtcttgagaaccctgaaatcagcagtgtctgcccactttgccctggaccagcctctctgtctgtatccttcccccagagaaaggaccatgggctgacctgggccaaggacacctggctggagccccttctctgacattaactagctccgtgacctggggaaagccaccctccctcctgagcctcacttttcctgtctgtaaaatggcctccccgtcatcacacccacacaggtgtcttgaggagtgaatgatggtgtgctccaaggcccccatggagagcccggcacatggcaggcactcggtgttcaagcactgtgttcccatagactctgggctcctgccctcctcgcccccaacccccttcctaggaaccagccccggcttgtcagagagccagctagctgtcaaccccaccccagctggccagttaatgagtgaccaggactcctgagcctgacctataaggaggtgctaggcagggacacagatgggagacggtggacagcggcagggggaacccagggagcgcgatgggctgcagggctgcatcagggctcctgccaggagtggccgtggtcctcctgctgctgctgcagagcacacagtcagtctacatccaggtgagtcccttggccagcgttccctttgcctgaagggccccatggtgggaggctaggctggagagggtgtactgggaattcagaggggcaccgggggagcacggggcccggggctcagcccaacacccatcacagcccagagaccaaggcagctggtgggtgggggcagccagatgttgatggcagtgatgatggcaatgacaacaccaaactgattcagcgctgctcccctcaaaggcccgtgctggtcacagaggtcagggtgtggaacagaaggctcctccttgcatgggaggaggccagaaagggaaggagacaccctgttatgctcccacagttccgtatattcacccacatgtaggtggaggaggacaggctccagaaaaattttcctgagaagtaacccttgagctggcattgcaagatgaacaggaggttgccagacacagatgatggggaaactgttccaggccaaggggcacagcatgtgcaaagaccagatagaagactatgggggttccagtgcccttccagggggcatgggagagtggcagtagtcgtcacagcagggcctgtctgccacgtcacaggggatgctgctggcctcgcccagcatagcagagacgtctgtggccctgggtgtcacatccctgactctgggcatggtctccttgggaagaacaaagtgggtcccagagaggggtgcaggagacttggcctccccaacagccctggcctcagagaccagctacttggggctgccatcactctgccatgtgagctcaggcaggcgtactcagttagtgattgtgccctggtaccaggcccccacgggagatgtcagatgagggaactgaggctgagtggggaacagacaggcagccggtcatgggcagaggtgggatttgaaccagggcttgtctgtctctaaagcctcgaaccactgtgctcca

SEQ ID NO: 20 - DNA, 2001 bp, Homo sapiens
ctatgctacaaaggcagtaggccaaatttgattgaattgaataattccttattttcatcagcttctcctttttttttttttttttttttttttgagatggagtattgctgtgtcacccaggctggagtgcagtggtgtaatcttggctcactgcagcctccacctcccaggttcaagtgattctcttgcctcagcctcccgagtagctgggattaaaagtacccaccatcacgcccggttaatttttgtgtttttagtacagatggggttttgccatgtgggccaggatggtctcgaactcttgacctcaattgatctgcccccctcagcttcccaacgtgctgggattacaggtgtgagccaccgcacccagccagcctctcagttttgaacatgcactaccaccacctccacaacacacaaatgtaaatgcactttcgtatataaaactgtataaatacaaggaagctcatacacatgcaaggatacacacataagcacccccagattcaaccacagaaatatacgccagtacatttgcataaattcaaacacccctttacatgtaaaaatcatataagcacatacagggatgcaagcaggcatggacaaatgcatgcaagcacagacaaacagacaaagctaagtaaaaaagtgcaagctcacctatgcttacaaaaatagacatacatatacccacaaacccacacacccacacattcacttgctcacctggactttgatatctctaccactgtatccctgccaatatctacagagtgggtaaagggataggcatcaggtcactgggttgcccaagcaggaagtctgggttccctaacaactttttctaagctaatgctcctggatgatgatgaaaaaggaggtggggaatggatgaaattttataacagggtgcagaggcagggtcaggataaaaggcccagttggaggctgcagcagggtgcagggcagtcagaccaggaccatggaactcagcgtcctcctcttccttgcactcctcacaggactcttgctactcctggttcagcgccaccctaacacccatgaccgcctcccaccagggccccgccctctgccccttttgggaaaccttctgcagatggatagaagaggcctactcaaatcctttctgagggtaagacacagacgaatggggtctgagggtgagctgcttcttgccttggtacttggggaagcttcaccaaacagaatgaggcagacttccagagtcaggggtggcacgggcatggttggtgagtacggagcatggtgaagcatgatgggtggtattattaggagaaaagcatcaaattaaatttagcagagtttatttgagcaaagaagtgactcatgagttggacagctccctaaaccaggaaagacaccacacggcagtatggtcaagtggtatttacaggcagaaaaaggaggtgacatacagaaacagcctgattggccacagatcacagcttgccttacttggtcacaatctgagcagtttgcagcctgtgtggactgaaagcccagctgctctgattagccaacacttggctacttgtcacaagaatatattcatttgggccaggtgcagtggctcatgcctgtaatcccagtgcttttggaggccgaggtggtggatcacctgaggtcaggagttcgagaccagcctggccaacatggtgaaaccttgtctctactaaaaatacaaatattagctgggcatagtgatgcgtgcctgtaatctcagctacccaggaggctgaggaaggagaatcacttgaatccaggaggcagaggttgcagtgagccaaaatcttaccactgcactccatcctgggtgacagagtgagactccttctcaaaaaaaaaaaaaaaaaaaagaatatactcccaagttaggttgcagttcactctacagagagagctttaggtcaaatttaatttaattaaacaattctccccttttggtcagcctca

SEQ ID NO: 21 - DNA, 2001 bp, Homo sapiens
gtggcgtgcacctgtaatcccaactactcaggaggctgaggcaggagaatcacttgaacccgggaaatggaggttgcagtgagccgagatcgtgccacagtactacagcctaggcgacagagtgagactccatctcaaaataaataaataaataaataaataagtctcatgaccttgagtggggtctcgtacccctatagatttcagtcttctcatctgcaaatcgaggaagacagaatcatccacctctcatttgctgccaggcctgagggtgtatgatgagtccactcagatccacacattttagtgtgttccctccctaccagtatttgcattttcactgaagacaaagtcttaaggtgaagcaataaacatcaaattggggaatggtcagcctcttcccacaaaagggcaaggtataaaaagaggggatgaggaggctgtcctcttcacctgctaactccatgtatgagtggtggtcacaagagaggaagcgggcagcagaaatcctcagctcccacagaaatgaatacattttccaaaataaagtcaagccaagcagccctaccccacagaaagtcctagcaaacagaggtggcttcctacctgaagcaccggccaggtgtgtccaacagtaggagctgcagtcagagatcagagtgacttaacagctagagggcacttgatgaataaacggaaataaggaaaccacatcagatgacacctcccttctgagccccacccatgtctacatttggggaagaacagtttaagtcaagggatcacagacttgtgattagagactgccagggtccatatgaccaagtgggggtcccaggtgtgaagctggggttgagggtccattatctgaattctccactctatggatgatcatttttattattttccttttcttgaatttatttccatttgtttattcctaaattccctagtatatcacctgtggaagcttgcaacttcctgaccaagtataaagggcgaaggatttgacttacagcagagacttcagaaggagtcccccataggagggacttgggggcaatccagtgggaaagaggaggaggactgcgcttgagttgtgtttgaacagcaggcactcaatctctacttattttgcaggcttctttgaggtggcctagctcggattcaaggcattctcatcaaggatgtgagagcagttactgggatatccttggcttccctgttggtttgccaacgagagctccccttcctcctttgcctccaacctggtcttctttggaggttggcttcccagtgttctccaccttccacaactccaaggcctggcctcaatctctacttatttttcaggctttttgagggtatttgaatggaacacaaaccctcctttgccgccagccactctttcgcacaatcactgccaatgaccagttggtttaaatggccctgaggctgacggcataggccactcccagagtctggctggttagcagagagaccacagagctggggcagtccatgaatggaagagctgtggtcaaggtcactgcagcccccatctcagtaacggagaatggcttctctgggaacccaccttagctgcctccagcagctaatggctgtcccctagcttgagtcttctggcaccttcgtcacttctccatataacatgaaccctgctgtgaggcatttacaagtcagccctcttctagcttgggagccatgagacagcaagaaccttcttcaattcatgtttatgttctctttcagggaatatagtaggtgctcaatcaatggttgtggaataaatgaagggagacatttctcctgtgattcatggaccataagtcaccaccacgattcagtgtctattgcttttggctacacactaaagtcactggtatgcctgatagcttcacttccacacagcccagttggtctgcctggcattgcttctttccaaccttcctctaatgtttggagctaatgccagtctgt

SEQ ID NO: 22 - DNA, 2001 bp, Homo sapiens
tccctcagaaactgaagcatgttgcccagccagttgctgatgccctccttctcctggctgtggctcacggagagcatcttgtgaaagagacgctggagtcggcccacttctagccactcccggggccacagcagggagtagacaaaccttgggaaaagaaggtcaaacttgcggaactccatgaataactctcctgcctgtagcaggtcctgctccttgtccttcgtgtagccgaacaagctcaggtagccagctgtgaacaagatgtaatagcagaagcgaaagaggctgtcctcatgccagcaactggcatccagactccagcctttggacgtcagcattacaaaggacaggctgtccagcatggtctcattaagatccttcaagccatcccccctcagatgcttggtgctggctgagtgtatcatctcatggtccccttgcactgaacggtatccaaataccttcagcaccagtttttttgcatattgcccaaagtctagttttctctgtgtgtccttgaggatggagccaaaggagagggggtccatgacgaaggtgaagtactggccccctagctgcactgtgaacacatccccatgcttggtcctcatgcgcttcagaaattcaaacatattcttccggaaagccatggcatggccaagccagggcacggtacccttgtccagagggggctcccatggcctgcgttgtcggagcatccctggcaggcacaggtatccagcaatgaccaccagcagagctcccagcactggaccccagagaaccatggctatgctcctgcggattaagctctcgacacgcacactgttccctgggtgccagagagctttgggaggccttggaaagtccaggatgtaaaggtctgagcacctggaccttgccctgtggccgcaagttatggcaaataacactagtgagacagtacttggaagcagcaaaatgttcccctggacttaacacttggctgactggtcacgtaggggaggaggaggtaaccaggagaaagctcagagagaagcttccagctatggtcacagctgctcaccagcttcatgctttgctctgaccttttcagacagctcttgcaccttctctctaagcctgccttgagcccactcccacccccatagctgacagactatttatacccccatttactcatgcttttatttatttgccagttcagttggtagaaatgaatcaagcacttacaaaatctcacttccactctttcctcatctttagaagactcaacaaatgcctccattcctacttggcccccttggtcaagtaagttaacctcactgtggctccactccctcatctgtgaagtagggataattacagtgccttgtaggtgctgttgtgaggcaaaaagagataacacctgaaaagcacttagaatagtgcctggctcatgctagtgctccatatatgttagttagtatcctaaagccttttctcttacctttgcctgctcccctggacctcacagcagcccgtgcttatctcctcattctttccctagtctgtcttcctctctggtctcaaagatgcttgccaagggggcatgaccttgttgaagtcatgtctgctcctacctccatgtgcacagagtacacagtcaaaggcaagtgaagaatgactgtatgcccttccacaacagctctgacccctaacccaaaaaaagaggcattgatgctggggctttagatgctgagaaggctgagatggtggagggggtgttgactccatcttggggtatgagtgtgaatgaactcatctgaggaggccacagagcctcccagatgatgactcttcaagcagaggctgagccctcagtctctgagcctctcacccggagccctctcacctgcccaactgcttgcctatacacttctctgacaagaaggcagaatcctggaattgtaaaatgtcagagacaatagagacctcactcaatcacttcatacttca

SEQ ID NO: 23 - DNA, 2001 bp, Homo sapiens
agcctcccaaagtgctgggattacaggcatgagccaccgcgcctggcctatatacatgtttaaaaaataaagacagggccagcacggtggctcatgcctgtaatcccagcactttgggaggctgaggcaggcagatcacctgtggtcaggagttcaagactagcctggccaacacggtgaaaccctgtctctactaaaaatacaaaaattagcctggagtggtggtgcacacctgtagtcccagctactcaggaggctaaggcaggaggatcccttgaacccagaaggtggaggttgcagagagccgagatcacaccactatgctccagcctgggggacagagagagattccctctcaaacaaaacaaaaaaaataaaaataaaaataaataaataaataaagtatgtagagttttatttaataattaaccacacagattaaaaaatagaaagtagccagcttcccagaagtcttctgtgtgcaccttctaattaccatctctgtcatgtgtcttcagacagaagcacatataaaaacaaggttatgtattgatcaaatgatgaaaatgtgtggtcagaggcttgcattttccctaggagcaatggctcagtatttgctaatccggtgttcacagtgactttacagaacataactacccccaagtaatgggaatcaactgtatttttattactttggtgtacaaagaactttgagctctccatggcttgccacaacaaaagacttcctaggcaaattgtctactgctttgttccaagtgggggctctacttgaggctgtgggaccaagacaaaagaagcagcctctatgtggattaggccccttctcctggcagagagaaagcacaaaggagctggcagaaactcacagtgcaaaactcactatgcatatgctacatctgctcatactccaataggcaaagcacagctgacagcaaacccaaagtcaatgagacagaaatgtgtcttccttccacgggccgttctaaaattgcaaggtaatggacaggagtgtcctcttaaaggaaagatggaatgaatagttgtgaataataatataacccatcactactactcctgttttaattttttgaaaatgtttagatacatattatatgcttgtttaatttaaaatatcaaactttaccttgctcaggctctgcccctccacactgggccactcaaagatgcccaacttaccctacttgggctccaacagccatatcaggttgcattcgagaccagcctggccaacatggtgaaaccccgtctctactaaaaatgcaaaacttagccgagcgtggtggcgcacacctgtagtcccagctactagggaggctaaggcaggagaatcgctggatgcattgatgccctcttcaccctgcttaggctctgattcctgacacaagacagctctgttctgtggaacccctcctcactatgcttggcccaactccccacagccgaccactctaacttctatgtatcctcctcatctccttgggctcagaccctccatgtctggctacccacccccatagacaccctcctcaccctccttcagttcggacagtccacatcaggcctcttccctatggagacagcttcctcactttgctccagctctaaggctcctccctcatctgctcccctgcctggacactctgctcaccccactcaggctctcattctatgccagtctgcccctctagccccagggggatgcctttctcaacatgggcaggctggtctccatgctatgctgcttcttgcctcttcactgggcattctcatctacccctggctccagcacaccaggccagcagccctcctacagagacaccctccttgacctgttccagctctgatgcccacacggagccagccgtggctcccttccccttggggcaaacgcctaccttgctaaatgtgcctatctaatggctttaggactgactagttcagttagtaaagaaaaggaga

SEQ ID NO: 24 - DNA, 2001 bp, Homo sapiens
tgcaggtggccagccacccagtgctgactaggtgggaggcttccaactacctggctatctaaatgcccgctgcttcaccagctgctggctaccttcctgcttagggcagtgtgactcactgacggattgacaaggaggcctggcaggacccatcacccagttccatccctctaatgctgtgtccccaggctcactcctctctacccagttccccgacctgcttacttgggtttggggtgtcaacaggatccaggcatttgatgccacaagtgtcaggacaacatctcttcttccctggacactgccagtcactctggcactcaggtttcttgtatctaaggcactgggcagatttcttaggaggacagactccagctttgaaggcttttgaagagaaagtcaagaggtcaggaggaggctgggtactgaggacccatcatgcctcctgaacaaagcaccacttttgagggggagagacccttacctcagtagctgtccacaaagcccaaagggtcatgcctgcctgatctccaggtgtattgatgggcttgattaaattagacaaatggacaggggacgtttcacacccaagaaaagggcagcaggcttctagtcaagacctcccacccaagagcaaaacaaggctacacaaaggcagagaggaaggctgggagtgatggagatggagagaggtaaacaaagataaatagaaccagtgtgagagaagttgagagctaaagcccaaggcagaagcacagagacaaaatagcagaagcaaagacacaaggaagcagagtcagaaactaatgatttgagagtcgcagacaggtgaagaagaaggcggagtatgagagagagagagagagagagagagagagagagagagtgggacctgggctgacagcatggctgcatagaggaagcatttccagaagctcaaactgtcacacaaaccttggagctcagccagacatgtattccaccctcagggaggtctcccgaaactaagctgagggatcagagcctacagaaggggacacacccacaccacaaggagaccccacctctgaccctgcagcccagattagaccagagtgactccaacttacactttccagagccttccacagcccaaggtgccagagttcccagggcaagcagcaccaggaaggggaagaggccgctggacttcatggtgaaggcaggagtgactctgatggccaatgccaaacctcactatttattctttcaccagtgggtgtggccccaccagaattctttcggcctctcccagctactattgcataagcaggaaacgtagccagagaccagaaatcctgacaccaaggagattgtttatctttcctatgccaggtaaggctatgaggccaacacagcaactatcagaaaagaaagggaagagattggcctgggaagttggttgattaaagaggccatctcagtgtccatctgagccctaaacatcacaagatcccaggccattagccttataggtggagagagggaagaagtggtcaattgcaaaggtgctacccggacctggaactaaaaccagacacagactccctgctgagactttgtgcaatacattctattctcacattgctaattctgattctcacattactggactctcaggcattgtgaaactctgctgattactcaatgcagtttctttctcaaaagttcagctcgttcaaccaactgatacttagttccctgaaatatttacaccgttatatccccagtgcctgttctgtgcctgacacaggaaaagcactcacaaatgaaagctgaagaagagctccgctctccacaaatctctctggagctcagttgtctcagctgtgaaatgaaatgatactaacaagtcagcccttctcagagggttatgaaattaagggcatgaagggactctgaatgaaaagaacaaatgaatagtatcccttcccactaaagagaattgaaatgaatctgaga

SEQ ID NO: 25 - DNA, 2001 bp, Homo sapiens
gagatctgggaaatagtttaatcagacactagcatttattcttagctgctttcttaattaaataaaaatgagtataaattaatttactaaatgttcacaacgtaaaaacggacattaaaaaaagaaaaaaaatgccaaggtaagctgataatgtttttaaaaagtagacaataaaataatgtatgtaacttaaaatttgccatgataatttattcttaaaatggaaaatcagtaaaatactcagacttctagcataatatctctttttttttatttattattatttaagttttaaggacatggatgaaattggaaatcatcattctcagtaaactatcgcaagaacaaaaaaccaaacaccgcatattctcactcataggtgggaattgaacaatgagaacacatggacacaggaaggggaacatcacactctggggactgttgtggggtggggggaggggggagggatagcattgggagatatacctaatgctagatgacgagttagtgggtgcagcgcaccagcatggcacatgaatacatttttaaaagtgtgttactactgtcatttcaactgactagaaataccaccagcacgtgattgccacctcaaaggactgagaatgtaatagtagtctatgaagagcagtgtgtgtgatggacaatgatggggtagctccagtgactgatggcttcaggtgccactgacgggtaattgttgatgtattcaactgattttccgtgagcacccactttgtgccagacagtaaggactcggagataaaaagcagccctctgctggatacgtttacaggctagtggaggtgacaggcaccgtcagagaagattgtaacaagatatgtatggcatagtaagagagaagagatcatctaattctgccactgagtatttaagggcaggtttcacagaagagtttacatctgaattgaatcttaaaggatgacagggagttcactggatagacaaagtagaggaggacgtaagaggcaaaggacacatcatgtgtaaaggcatgaaagctgagccagcctattatcttggaaaaactacatgtactgtagttactacaactgtcacaaaggctattagtggagcagcagaggaaggttaaataacagagggaagttggatcatgaagaaccttatccatactacaaggtgattttatccagtaggcattgaaaaatgtaatcgaacctgtgttttaatggaatattgactaggatgaggggaaaagacagaggataacaaaacacttagtagacaaaagcaacatttgtttctatctcctactacttcagtcataattataataataatagcccacattattgatactgacatgtagataggtgaatccaatataaaatgataggaggtatgaaagaagttggtgagtttggaatattatttgactggctataatatagagtaatggggtgggagccagagggaaatcgaaaaggtgacctggaaaacattgttcagggccttgaatgctatgttgaggtattttgactttataaaatagtgggggacaaactacaagtattttatacattgaggtatagtttatgaagtttttttatatgtatagcttatgtaaatctcaaaggaatgctgagaagtaggcgttattatcccattgtacaggtgagaaagctgagtttgtggaatatgcatacattctacattattgagcacctgctatttgccaagcactatgctgagctatgagactagaaggaagttgtagtttaatactctaatagaggaaacaggaataaatcagcaattgtaagtagttgctataataaaggcaaacaatgttacgggagtgaaagggggacttgtctatatttgcatagtcagggaaggcttcctggaggaggtgagcttaaactgagtttggagaaacaaatagattacagtaattgcccagagatatttaactaggaagcagtttacctgaaacttgaa

SEQ ID NO: 26 - DNA, 2001 bp, Homo sapiens
aataggtgtcagccagtttgcagcaacctgtcttgcccctaccattcttgggttctgtattcagaccccagctattcctaacacccagttcatactccacgttctgtacaaagccatccctagctatcccagtcagcccacactggtctccatgacatgcatgaaccgaagtttttagaacctttgacacttatgtctgccctggatgatttgttcattatcttatgttgttgtcatccacagaacaatctggagaatattcatatatttagtcagcaaatagctatttattgggggcctttgtaggcctcaatgttgagaagaagggttggttgttttggacagattgtgtatttataaagctctagcaatttttaaatctctagcaggaaccaaagccttcttgacagccacctctctgaattattgtcactagcctccttaggagaagagacaatatagtttataccaagatttgcagttgtctagatattactcacttcagctagcactgtgctagtagataacattcaaagttggtctcaacaaatattttctctagtgcccatggagagtggctgagaaatacactctaagatgtaaaagagtttggtatctagatcctctttcttattcatttcaagccaatatttattaagcaccaactgcaagctagatactattatagttaggaatataaagtgggccagggatggtgtttatgcctataatcccagcactttgggaggccaaggcaggaggattacttgaggccaggaattcaaggtcagcctgcccaacacagcaagacctcgtctctacaaaaaattaaaaaattagctgggtgtggtggcatttgcctgtagccctagctactcaagaggctgaggtgggaggattgcttgagcccaggggttggaggctgcagtgcgctatgatggtgccattgaaaacaaaaacaaaaaccaaccaacaaacaaaaaaacaaacaaaaagaaaacgatactcagtctttatagggaggttggccagtcaataggttactttatgagttgctaaccctggtgagcaggaagttatgtggaccaggagagaaacccttggttcagcctggagaaaggagaggttgaccctaaactggagggtggagaggaccctgttgtgactctccgactgacttgtcttccttgatgtcctttaagccggagctgattcgggctgctgccttatttctgagttagcgctcttaagattgggcctcccagtttgaggaaggggcgggctgctgtctacctctgtgaatctgccctggaccaccccgggagagaaggagggctccggggaatctcgcacattccaggcaaaggctcccgggccgcagcctctgtgccacacccttggcccgggccaggtgtgcgccctcctcgctgcgagggggagcgggcggctgcggggagcgattttccagcccggtttgtgctctgtgtgtttgtctgcctctggagggctgggtcctccttattcacaggtgagtcacaccctgaaacacaggctctcttcctgtcaggactgagtcaggtagaagagtcgataaaaccacctgatcaaggaaaaggaaggcacagcggagcgcagagtgagaaccaccaaccgaggcgccgggcagcgacccctgcagcggagacagagactgagcggcccggccccgccatgcctgcgctctggctgggctgctgcctctgcttctcgctcctcctgcccgcagcccgggccacctccaggagggaaggtgagtcggcttccacaaggaaacatctcagccctgggctgagaatctgcctttctctgcagaccgtgatggctgaacgtacctccgaggattcccactttgtccagaaacttgttagagctcccttctcctcccctatctggggctcctccagagacggattttaaatgagactaatatataaagctatctgctttttaaaaatagtaaga

SEQ ID NO: 27 - DNA, 2001 bp, Homo sapiens
tcaggtgcagcgtcacagcagggggcctgcgttctgcttcccagtctgggagaagagagtgtctggatattttcaggaaaaaaaaagaaagttgagcagccacagcagggcagggcagtctacatcctcaagtctccctcagcttctctgctgctgccggctggcctcaaattttgcctctctttggttctctgagaagttttaccaagagaagagacaggaggagagatccagcctggactattgtttgtggggtgtgtaggtgtgtgtgtgtgtgtgtgtgtgtgtgtgtgtgtgtgtgtgtgtatgcgtgtatgtatcccgcgaggctggagccacagcttccctgatgaaccatggaggcgggccagctcctgagctgcggttgccctggagacccacctaagcctctttgcctggagcagcattatctgtagagggagtggctgatctgacacaaaagcctgaggcgacccaggcccaggcagtgagggagcacttgtgtccagatggcattctgtccgcttcctcctccagccggtgctcagtgtggcacaaactgggcaagaccattctcacccaaccctctgctgagccctaggtgtacctgctgttggcaaatgtttccagctgcctggtgggtggctcctggatggagatcccctctttctatcagctccctcctacagggaaggggtgattctgggtgctgaggacagggacctgtgaagatacatctccgcctttgccccaccacatcccttgcctgtcttcctctctgcttatctaaattatttccttccctcaaggcaagctcaagagcagacagttctgactttcagaaggaggggctgtccagggagaagaggaacatggaacagaaagcattgacgttgaggtatttgagataacatttggggatagcagaagcatcctgttgctgttctgcagcatcccacactcatgtctgggcctccttcctgtctctgctctctggtcacgctgtcacttgtagactgttctgcctagaagccatgtgtgggccagcttgcacctaacagacattaggtagcactaaaacagatgcacactgccctgtgaggggcatgggaatactgtaatgggcatgaagctgcattataatagatcttaggtaatcctgtgctgggtgatagcgtgacttttgcacacatctggttttgattgtgttggaaataaagcagaaagtacaaccaagaataaattaaacaatggctgatgaaatggactcaggttgcccacctgcctgcctgcatgatttcgtccctgggtgatttgtcacttcgtgcttgctctgtggctggttccactcagctggttccatccgccttccctcttcatccatcaagcaggcaagattcttgagtcaggagttggatcatatacttctcttgaccagttcacagcacgacagccccaaagattgagtgggtcacttggggatggaagatccccacatttgagggagctgaagctgacttcctctgacccacagttcccaggcggcctccacctcagggccagctaaggccagagtcacccaaagcaggtaactgggagtccccacctctgagcaccccaccactatcctggcactttcccaaggggcgtggcttggaggtccaagggcagaaattcccaggcatttccacactgtagggaagggagcagggggatgacaagactttaccttccctttaggaaagttgctggaggcaatgaggagaggagggcatggactaagtcacatgctcatctgtgcagcacgtatttctaaagcaccattctacaggggcaccaagctaggagctggctgagcctggctctcctctctgcacacatcccagccaagcctgtgtgtgctggattgctggatcagatgaggaggaggtggaagtctggcaggcaggtggggagcagggacagcattagggcccagagttaaagacttttatctccaag

SEQ ID NO: 28 - DNA, 2001 bp, Homo sapiens
acttgggagacagagggaggaggcttgctcgagcccaggagttcgtggctatagtgagctatgatcatgccactgcactccagcctggacaacagagcaagaccctgtctctaaaaaagaaaagaaatttggaaatggtttattttgtattaacaatttataatttacactgaaatttattatgataaaacttttccctgtgttaaaaagctattaactttatgaaaaatttcttttaggtaaggttgattatatatacccacacacatacacaggttaaaagttagtttcatgtgacataataactagcattttgagcactacctgtttgcccagcactgttctaagtgctctacatgtattattgttaaattatcataacactatgaattatgtactataattaccccagctttacagatgaggagactaatccatggggaggttaagtaacttgtccaaggccagacagctagagccggcttttggacccacaccacagtctgactccagcacccatattcttaacaatttcaccatattaatatgtcaagattaagcagttttaaaggatgctattttctcacaaatttcttaatatgaacactcaataagaataatcactaatataagcatttagtatttttttaacactaagttggaagcatagtggaacatttatttttagaaatattattaattggctgggctcacgcttgtaatcggctgggctcatgcctgtaaattttgggaggccaaggtaaaagaattgcttgagcccagtatttccagaccagcatgggcaatacattaagacatcatctttaaaaaaaaaatgttattaatctcctctttttgttaaatgtatattatcaaaattgttactaagctaacaaacttcagaaaaacttatgatgggcaagctgcttgtgacattgaaggtatttaagattcaattctagtttggtcctagatgaccacatatccattgttccttcaacgagcacatggtaaagagcctagaacacagagacacagaacacagtggagaaaagggagtgaaatgtctttaatgacacttactatatatgggattttgtgacaatatacaaggatggttaagacatataaggtgatgcaaaaaaacatattaacaattatagtgacaaaaaatgaggagcatataattatacattgatttatacagagtaccagaggaacacagcattgagagccgtaacaccacctgagggagtggagaaaggcttcagagagaaagtgttttttggaatggatcactgtttccaaaagaactaaagtacagtttgagaaatgcatacttaattcattacttttttcccctcaactttaataataaatttacccaacaaaaaagtttatttttgacttgtaaatctcttaaaatcataaaaaagtaaaattagcttttaaaaacaggtagtcaccatagcattgaatgtgtagtttataatacagcaaagttaaatacaatttcaaattacctattaagttagttgctcatttctttgatttcatttagcattgatctaactcaatgtggaagaaggttacattcgtgcaagttaacacggcttaatgattaactatgttcacctaccaaccttaccttttctgggcaaatattggtatatatagagttaagaagtctaggtctgcttccagaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgat

SEQ ID NO: 29 - DNA, 2001 bp, Homo sapiens
tttcatattccatttctctgtatgctttcttcaccaggaagaaatagttttagtgtcaggaatgaatgagtctgcccctcaattccagcctgctcagcacacaaggaaacaaagccctgacaatcagagtgactccctggtgactaagctccagtcctggatgcatatttgtttagcagttctgacagcatctgacccagccctctctttgcataccccaccagaaccttctttttttttttttttctttgagactgagtcttgctctgtcggaagcgattcccgtgcctcagcctcccaaatacctggaattataggcgtaagccatcatgcctggctaatttttgtatttttcatggagatggggttttgccatgttggtcaaattggtctcacactcctgacctcatgtgatccacctgcctcagcctcccaaagtgctgggatgacaggtgtaagccaccatgctaggctcagaaatttccttttataaaaatgtcattaaggatcttggctgcacaatatcgttaccagcttcctttaaatccacctctggcctgccaggaatcagggttcttcagaacctgacattttaaatgaagaggtcaggcaggtcatgaggaaagcctcattgtccccatgtctctgtcactgctgcacccctgagacatcacagacatggacactggggcctgcttgtttctcaaactgcccttagatcgaaagagggaggaaccaggatgaatgccactcattttcccaagaaaggccctctcctgagtgcccgggatggggctctgtccattgcctggggccgccaattgctactctgggttacggaagaaggacagggtcctgagagacaccagagacctcacacagccctgaaaacatggggctccttcataagtgtttcccatcaccaacagggagaccacgtggaggccttgcagccctactcggtgcttctccaccaaatcccaagggcagtgacgctgacgtctgtggaaagcagagaaagccctggctcccaaagccctgaagtcctgtggagctgacattccctgagtgacggtgtgaatggaaggaactcaagtgcgggtggtaggccacctcctggcccaggcctgggtgaactctgaggggacacatgtagtcacaatcccatcctcccattctccttctcagaggaaggaagtgggcatccatctgcctcatctctctcccgtggggaagatggggagtttcaggggaactttcacataaatttcaccagctcagatctcctgtgaggatggggcccaccatgctcccggtgctgccagaggccctgagcccctccagggtccctgggtttgagccagccctgtatcatccccaggagctgaatgtccgagcaatggatagaattagatggaaagagctctcaatttggcctgagactgtccccagatactcaggaaaaacaggacgtcgcacagagtgggcagcaggtgagtggcaggttataggtcctgagtttgagtttgttctcacgtgagacagacccagcccctcactccattcacacactgggttttaaatggtgcaagataggaggaattttctggtcccaagagcaggaggaagggattttctggggtttcctgagtccagatttgcataagatctcctgagtgtgcattgttctttgaggaccattctctgactcaccaggtaagtggctgaattctaacctctgtaatgagcattgcacccaataccagttctgaactctacctggtgaccagggaccaggacctttataaggtggaaggcttgatgtcctccccagactcagctcctggtgaagctcccagccatcagccatgagggtcttgtatctcctcttctcgttcctcttcatattcctgatgcctcttccaggtgagatgggccagggaaataggagggttggccaaatggaagaatggcgtagaagttctctgtctcctctcattc

SEQ ID NO: 30 - DNA, 2001 bp, Homo sapiens
gaaaggaaagcctggctcccgctccaactgcaaaaggagaaagataaaggatggggtggaaaggctgggactggaaaaatgtggagagggccaacaaggggcatctggagacgggggaagaagggggttaaggagaggggcggagagaggccaagaggctgagaggttagggcttgaaagagggcgccggcgagcaggagaaagcccaagaaggagtcggaggttggggaaggaggagataaaggaaaaaccccgggatggagtgaagacggcggcagcaagcaactcaccagagcccagggagaggcagggctggggacggcggagagccgcagacgccccttcggccagtagtctcgacttgaaggctggcgaagccccggcggcggcagccaacgagccaaagacgggaggggacgcgtgagggactctgagacagggggcaacaggcggttacgggtgaggacaacaggaagcgttccaactgccggaaaagacgcgagcgctgaggggccaaggcagcccggtcccccgccgcggagacgggctggcgcggcagcccggggccgagactacgcatgcgcgacttcccggagcggccgggattgcggaactgcgcttgcgtggccccgccctgcccctcccttcaccccctgcctagggctggctggacctgggtgaggccagatgtgtgagtgtgaggcggtctcgaagtaggctgggctgagggggaagttccattgttttcttttgttttactttttacttcgaaataattatggactcacaagaagttgcaaaattaatacatacaggttccctataccatcacccacccagcttacctcaacatttttcttaagatacaacattctcaaagccaggaaattgacattcgcacaatacaattaactaaaccaatacaattaactaaaccaatacaattaacttcattacagaccacactcggatttaacctgttgggtatgcagtcatgtgcgtgtgtgtgagtgtgtgtgtagttctatgaaatgttgtcacctgtatagttcccacccacccccctaccccgtgtagaacaggtctgctggagcccttggcccccctcctcttccttaaagtaaggaccttacctgggataaggtgagctctcataggtttgggaggttacccagcgggcgacccagtgtggacagaagaaggtgcttttcaaggccacccaactcttattctgtactacttttgggggtaaagaggtcccaagtttaatagcctttgggcacttgtcgtttcttgagaatggagcacaaagccctcaactacaaagtgtcaagctcctggaaactaagatgaacgagacatggtccctgactgcctgcaaaggggccatctagggagagaaggaagcatgtgctgcgagttattcattcatttcatcttcaaagcccagtttccttttgtctagcctcttaacaaaaatcacttctcgccaggcgcggtggctcctgcctgtaattccagcactttgggaggtccaggtgggaggatagcttgagcctaggagtttgagaccagtctgggcaacataaggagaatccacctctaaaaaaatttttgtaattggccagcatggtggcaagtgcctgtggtcccacctactgtggaggctgaagcaggaggattgattgagtccaggagttcagggctgctatgagccatgatcatgacactgcaccccagcctgggcaacggagggagaccctgtctcaatcagtcactacatacatatcaatcaatcacttcgcctttcattctctctgcccagaggcccttctccccaaatgcctgttgtcaaagtcctacccattcttaagcttttaataatagtccaagatgtctgcataatgctttacagtttacaaacaactttcacttatttaatctcacttggtcctcactttggcagtgacacacgaggttttgcagatgaagtaattgata

SEQ ID NO: 31 - DNA, 2001 bp, Homo sapiens
gccgagatggtgccactgcactccagcctgggcgacagagcgagactctgtctcaaaaacaaacaaacaaaaaaaacaaaacaaacaaacaaacaaacaaaaaaccttccttggatttttcaagaagttttcacaattgcaaaaaaacaaaacaaaacaacacaacacaacaaaacaaaacaaaagcaaaaaaaccctatgcttccacccaaggtaaaaattttagctttaggtccactctcaatacattatttaatgaactgaagttggcaaatattcctcacagcctgttggagggttcagcagtttattacagaagtatgaaatgcttttatttaaaaaatgtattttggtaaatacatttttgtttaggtaaatatcatcaaataatccaatttggaaaccaacattcttacttctttttccaacagttgttcctatcatcataaaaacatgttaaatttttctcatcctttcaaaaaatctctggagcttacctcaccctccagatacagcctcacctctcattccaccatgaaatcagacttcttgagatggtttcagctggactccatccttcaactgacaacccaacacaactgtattcatctctcgttaacattactagcagtgagtaacatcagaaagtttttgacacatgttagtcttttttgtgatgaacttcacagatacatttgacattggtatgcctcatttatttgttgaaatttttttctttggcttccatgaagtttctttctcttctgtatcattctacttctatgactgctccttctcgagtaaaacagaatgtgtctcaggattactttaaaacaagacaaagtatagagttatacctaaaatttagtatttaagttattggatcagaaaggaaactcgcatttagagtatgaaggcattgtcagccaccaattacttttgtaacctgaagctagtctccttcctactccggactgaatttcttctgtataatgcaagcgatctggcatgatgatatacaaagaccgataaaattttgctggggattctgaaagtaaaaaaaattgcctttgatattatgtccccatgctaagtccctggggactttgacattatcccccactgagcaggggtgaggaagctggcatttactaataagctatgaaagggcagtgccttttgacatttcagctccacccatagcacacccactcaaggaacatataaatgaagagatccgctcagttctcagacaagatttttcaagcaagatgaagtccatcatcctctttgtcctttccctgctccttatcttggagaagcaagcagctgtgatgggacaaaaaggtgagtggagagggtaagccttggggaaagctactttaaaaaaatggcctctaaggatattcagggtgcaaacagtaacctgttcaggcacagattcttctccttgatgagaattgatttttctccacccaacgctgtaggcttttggaaatatcagaaatttgttgggaaaaggtgggaggtaagagttgcaagagagctttggagataatgaatgcatacatttctattatcaattaccaggtggatcaaaaggccaattgccaagcggatcttcccaatttccacatggacaaaagggccagcactattttggacaaaaagaccaacaacatactaaatccaaaggcagtttttctattcaacacacatatcatgtagacatcaatgatcatgactggacccgaaaaagtcagcaatatgatttgaatgccctacataaggcgacaaaatcaaaacaacacctaggtggaagtcaacaactgctcaattataaacaagaaggcagagaccatgataaatcaaaaggtcattttcacatgatagttatacatcataaaggaggccaagctcatcatgggacacaaaatccttctcaagatcaggggaatagcccatctggaaagggattatccagtcaatgttcaaacacag

SEQ ID NO: 32 - DNA, 2001 bp, Homo sapiens
ctgacagtgtcttcctgaatgactcaagtggtcaaaaacgcaagattatttttcatttttttacagagaaataaaatacaattcatggcctttactgaagtttactgatttgtgattcataaaacaattagcgtatcattagattttattaagagctataatttagtgttctaggaaatttagaaggccgaattatactttgaaatgcactggcaggcatactaaggaagtcctgaggtgaatcattttttataaacctacatttttatatattacattagagccctataaatatatgtatacactttttttttttttttttttttcagacagagtctccctgtcacccaggctggagtgctgtggtgcgatctcagctcaccgcaacctctgcctcctgggttcaagagattctctgcctcagcctcccaagtagctgggattacaggtgggtgccatgacgcctagctaacttttgtatttttagtagagacagggttttaccatgttggccaggctggtctcgaactcctgacctcaggtgatctgcctgcctcgatgtatatactcttttatacttacttttgactagagacaattgagacttgaaaattagaaaaatcaagttagaattactgagaaaattagtagctctgatattctacccattctttatttttaaattaaaattaatcttaatgacctacaaataggttaacttaaacagtccataccttacatgagaagctccgacttgggatgtttcttgctaactctgatatcttgggtttattctgaagacacttggttaggtgatattttttcaattttattctattgtaacaatgatcagcgaagttatagtgattcaaatgctgtttctgcatgcattttccaaggtgaggaatactatacttcaaagcttggagaaataatttggtcttctgctgcatgatatcaaagtccctggtttatgctccttttggggctgccagcacacgaatgattttataggtatgattacagagtagtgggttctcagagtaactaagatcagcagttctcatccctccttttcagggccatgccccacttaggttatgaataacttcagacagctgataacttaggctaactaaatctaaaaacctgtgtaagttaaagtttcattcaaatcataattcttggcagatgtgacttaaatgtctcatttgtaaagttaatcctatcaggctcaaactatgcagcactgactctggcacacataggggttaaactaaaacaaaaaacaaaacaagaaacaaaatataaatttgcaaataagtacacagacttaatggggtatctcaggaactcagggctgtaaaaggtaaaaagccctttggtcttacataattatgaacgtatctttggtatatatgtggaagataaactggaaaagcctaattatttaataaaggtgggggagggaagattaattattgttgagtgttaactaactgccaggtgcttttatcttgtttgtctaatttaaattttatatggacttaataaagtagatattaccatgcacattttacaggttgccaaactgtggctgtgggaggttctaagcaactgttccaggtcacttgtacagtaagctgtatagcaaaaaatccaatttggttcggttccaaatactggacatactcaacttcggatttttaatgttaaaaaggtatttagcgctttaattaaaaacaacaaaaaacccctgcaagatgcacaaataaaatgtaaggccatctctggtttgtcaggaggtagcttttattaaccttgattgctaataagtagttaagagtcagagagacctgggttgaggtcttagctcctacacttaactgtgtctccgggcctcagtttcttcatctgcaaaaatgaaatgacaccatttaccttctagggttgttgttgtaaagacaaaatgagacagcttaacattaagaccatacagtg

SEQ ID NO: 33 - DNA, 2001 bp, Homo sapiens
ccccagtttccttggttaaaatgtatatgtacataatccctaaagtgctggccaagctcccaccatgagataagaatttcaggccaacctgactctataatggatggccacaggaaagaacatgctagttgtctgtgatgccaaggactgcacacactaacaccacctgggcacacactaacaccacctgggcaaaagccattgggtgaggagctgcatttttttttctttgagacagagtctcgctatgtcacccaggatggagtgcagtggcgtgatcttggctcattgcaacctccgcctcccaggttcaagccattctcctgcctcagcctcccgagtagctgggattacaggcaactgacccatgcccagctaatttttgtatttttagtagagacagggtttcaagttagtcaggctggtctcaaacccctgacctcaagtgatccacccacctcagcctctcaaagtgctgggattacaggcgtgagccaccaggctcgacctaatttctgtatttttagtagagaccgggtttcgctatgttggtcaggctggtctcgaactcctgaactcatgtgacccgcccgccttggcctcctaaagtgctgggattacaggcatgagccaccacgcctggccaggagctgcatttttatgccattataacttttctagttatttttttctttttctccaccagacaggagtcaaaattaaagtcatctctgttttttaagaagtaattccaaaaagaccccaatcaaacctaattccttaggattctgttttctgaaatcctaggcatgacagtccttaaaccagaatgattgctgctactcaccatggctgggccatgcagggtcttcactgtcatgttgcgctggtggcttactttgtgcaggaagctctttctgtgaatgtatcttcctgtgtatgcctgtgttctatcaatgagagtacctgttagttttatagcaatcattgcccctgtttcgattgacctgcttactgtctttaagttgtggttgacccggagttactgacgaatgcaggggtttttttttttattccatttcccgaaggggaacaaaagggggaccctaaagaaagggatgacaggaaaaggaccaaatttttctttccttctaccctacttccacccccattgcccacaaatcagagcaaaatttttaatttactacagagatgaaccaagttagaaattgttgtcaaatcttttttgttcttccacaaattgccaacacccttgtcacggaccatatgttctactttccagtcacgcaatatgaatcgtgatttaaatgggagaaaactcatccttagaggagccttggtactaaactatctttacctccattttatgaatgggaaaacaaaactaaagaaatgaatgacataagtgaaaaattcagaaatgatataccctggtagttttgttatctccctgaactaattattttttaaccccatggttagactcctactggtgcttaataaataatcactattcattgtgttgataaaacaaactgttttcctctgctctcacaccacaaccagcaacacaaaagatttccatgaccatgtatgtgggcatttatctccatcaaccagcaagcaatcaattctgcatccaacgccagctaagcatcttctaatccaattcaattctgacgctatctccaggtaggtagagtcagatcccacaggctgaaggctcagtcccacaaaactgctcccactttaagcaccaatcacaggtccaagcctctgggacttctgactgactggcctcaagttcaggttcccacaaaccgcttgagtttgattaggcttgattaatttgttagaacagctcacaaaacttagggaacactaatttatatttaccagtatattctaaaggatatttttaaggatacaagtcaacagccagatgaaaaggtacacagggcaaggtttgaaagggtcaca

SEQ ID NO: 34 - DNA, 2001 bp, Homo sapiens
ccaccaactggggccgggcgcggtggttcatgcctgtaataccagcactttgggaggccgaggcgggcagatcacgaggtcaggagatcgagaccatcctggctaacacggcgaaaccccgtctctactaaaaatacaaaaaattagccgggtgcggtggcgggcgcctgtagtcccggctactcgggaagctgaggcaggagaatggcatgaacctaggaagcggagcttgcagtgagccaagatagcgccactgcactccaatctgggcaacagagagagactccgtctcaaaaaaataaataaccaattggacaagttatctgacttttaagaactttcatctataaaatgtcaatactagctaccttatatggtgattgtgaaagttgggtaagtagacagtgtgaaagtacccagcatagtaaggattcagttaacatttatggaaaataatccagatacttaggaacttttatgaaataacacaatgtatttaaactgttttctagtctgccatttgctttacaattataccgttgactggtatggtgtttcccagtgtctcatctctcttttaggttactgcttgccttccttgtttcactgaattggtatgtctgacctaatgaagactttttagattatttagaaactactacgttttctgcagtcttacctgtttcagagcgtggtggagaatccaaacattcaaaaacatgccatcgaggtgtctgccctatcaatgaggaaaaaggcatctggcaatttggacagtatccatcataaactggacgtatctttggggacacgtgttggcttttttgagttctacagagttttcctggagtagtttccttgtcttgggtctgctgaataccatctccacaacttgaattctgactagaagcaacagaagtctgacaacctgcatttccaaggggcacttcatggtccttcacctctttagcttctgcggctctttttctgtttctactccgttttgactggtattttccatctgttgctttttcaacagattttagaatatttttagagccattatttggatcaactcgttttggttttcttttagatttgtattcccaaatgtcttcttcggaaatgtcttctaacatggcaaaatgattttatcattcaagaagtattaatcttaagtaaactgaaagtccatttcttgtcacaaacaaaaagttatagaattattttgctgagaaaaaaaacaaaggtaacaaaacccccaccaactcaatgggaatctgcagtgttggtaatgaacatattggcaagctacaaaccttgtacctgacaacacctgctattccatttataccacaatgaaactaagataaacctatcacaggagtagcaactgtgaagttctccattatgggtgctatttcattcaaatatcttgtccttttccagggcttgattcataaaaataatacttcaaaaattaagcgctaatggcaaatataggttcaacagcttaatcttagaacgagatttgtaacattgttatgaagctactgtcagttctttcaagtggacaaccgtccaaataattcataaatccaaccacagtccctggacaaagggcttaacaggtatcccaaacactaaatgactatctaacaaaactagtcacaacataatcaaagagatgtgtaattcgcaaaaaactgcgaagtcactgccctcatccacgactagtggatactcaaccacttcagtttcccgcgacagtccatgatcttcctccagcagcaatgaacaatccctgcacagtgaacaaaaaaagttgatatgtgaaaaaagttcaaatatgtgaaagagtgggcttgtattgaggtctgactccgtattgcacccatggttccgaaggtggccagccctctgtccacctgactcaaaacggggtcacacgccagcctctcccttatcttttggcattagaggctgggaggccaggaagcg

SEQ ID NO: 35 - DNA, 2001 bp, Homo sapiens
gtgatccgcccgccttggcctcccaaagtgttgggattacaggcgtgagccatcccacccggcaatcgtcaggactttggcttggactgtgagtgagatagggagttgcaggaagctttttcgaaaaaccacctttgggccaggtgcagtggctcccacctataatcccagcactttgggaggccaaagcaggcagatcacttaaggtcaggaatttgagaccaacctggccaacatgttgaaaccccatctctaccaaaaaaatacaaaaattagccaggcatggtggtgcatgcctatattccccgctactcgggaggttgaggtgggaggatcgcttgaacccgggaggtggaggttgcagtgagccaagattgcgccactgcactccagcctgggcgacagagcaagagcctgtctccaaaaacaaacaaaaaaacccaaaaaacttcttttggcagtgtacaattattattactattttttgtagtaaatttacagagatctgcagtcatgaccacaatccaaatttagaacaattctatgacttcaaagggatgccttagagccgttccctggaaccactcctctgttctctctgtatctactggagggttctaagcagaaaagaaacaaagtctgacttatattattatcactattgcaccttttctgttaaaacatcataatcccagcactttgggaggccgaggcgggcggatcacgaggtcaggagatcgagaccgtcctggctaacacggtgaaaccccgtctctactaaaaaatacaaaaaattagccgggcatggtggtgggcgcctgtagtcccagctactcgggagcctgaggcaggagaatggcgcaaacccgggaggtagagcttggggtgagccaagatcgcgccactgcactccagcctgggccacagagggagactccgtctcaaaacaaacaaacaaacaaaaaaaaaaacccgtcataatcttcctgttgctttttccgattagatggcgacttgaatttttagggaatacatccaccagtttgggcacacccaccaggcacgacagttgaggtctgatcttccagccactgtgagtactgagaaaacttggcagggggccactctcactggctcaatgcaccctgaccactggaagccactgtgtcagctgctgggttctggctctgcccactcttggctgcatttggggctgaagttccctgtgggaggctgttttctgagggagctgagtgtttacagccactcagccctgctctgctcagctgaagcagaaaacagagaccttttgcattactttggttcaagagcaaggtaatttggaaagaaatttttcagacgaaggtatcagctgcctctgaggcctttgctgattaaatcaagaagtgacatctgaaatggtgtcggggaatgggaaggaggcctggggattctaaattatcagctgtttaggtggctgaggaatccaggggtgggagactgaggaaaaggactggggcagagctcagttcaggagaggtgaggggcgggagaggtgaggggcgggagaggtgaggggcgggagaggtgaggggcgggagaggtgggtcagagaaaggaagtctctgaggctgcaggcctaggcctggtggccactgcagaggctggggcctatgtttttccaggaagtgaaacctgaaagtcacttttcccctttaagcctcattttctttatctttaaatggagaaaataacatttacagcatagagttgttataaagatttcatgaaataatgcctctaagcactcttaaaatgcttggtacacagtaggccttcaatagactgaagctgttatggtttttcttgttgtgccacgttattattgttggttgttattaccatattttgttattattattactaagaggaatttaatgcaaggaaactgaaactgaggtcacacgcagctcccgggccagtgcctctcaaactttaaca

SEQ ID NO: 36 - DNA, 2001 bp, Homo sapiens
ggggtgtccatttttttggcttccctggtcacattggaagaagaacaattgtcttgggccacacatacataaactacactaacactaatgatagctgatgggcaaacaaacaaaacaaaaaatgctaaaaatctcctaatattttagttaagtttacaaatttgtgttgggccacattcaaaactgtcctgggctgagggttggacaagctgctttaagtcatttgtagattacttataatacctaacacaatgtaaacgcaatgtaagtggttgtcatgttgtattttctttgtattattttactgttgtattattttttattgttagtttttgttttgttttgtagtattttgatccgctgttggttgactctgcagatgcagaatgctcggataaggaagccaactgtactcccgcataccagctacttgcttactctctatttattatctgttattctctatgccttcattctagcatatgcttcattctagcataagtaatcacctttctacattctggagtcaggacaatatctagatcattgctcaagagttctgaactgggagattaaaaagctgggccagctgggcacagtggctcacgcccgtaatcccagcaccttgggaagtagaggcgggtggatcacctgaggtcaggagttagagaccagcctgaccaacatggagaaaccccgtctctactgaaaatacaaaattagccgggagtggtggcgcatgcctgtaatctcagctactcgggaggctgaggcaggagaatcacttgaacccgggaggcagaggctgcagtaagccgagatcgtgccattgcactctaacctgggcaacaagaacgaaacccgaaaaaaaaaaaaaaggctggggaggtgattgtggggaaagacaggtctcccaaaatgccctctgtgtgtagttgttcacggaaaccacataaacagaaaatagctacaacagctaattcttactaaggtgtcaagcgttgtgcataacactgcctaatgtgtgacattaaacgttttcaccaggtcagccacttaatcttcagaaaactggtggccaactctgaggtttcacattgacgtttaatggctgttcctaaagaagtgcgtgctttttaaaaaaatagaggatgggaggtaggttggaaagggaagataagcggcaatggaaaaatcgagtgtgattattgcatttttatcaaatatgtctgtctggattatgtggttagaatcactgcaaatttggcttctataaatactttaaaacttttttccaatgttttcattatcagatccactatgcaggctttgagtgggctgccatttagttcagatatcaaagtattttacaagtaactaatagcatttattatctacacgtggaaaactatagcaaagaaaggtcaatcaaaagttgtgctctaggtcatcagataaaatgagattaatactgaaaaattcttccacgaagaccaggggtccagctaaatgtgtggtggtgggaaaaatgtttatctgacgtccctctaaagaaacctcagatgggagaaaaatgtcccagccccaggaatggacacatttaggtttttccttttttttttttttttttttttttgagacagggtctcgctctgttgcccaggctggagtgtagtggcgcgatctctgctcactgcaacctccgcctcccgagttcaagcgattatcctgcctcagccccccgagtagctgggattacaggcgcgcgccaccaggcccagataatttctttgtattttttagtagagatggggtttcgccctgttggccaggctggtctcgaactcctggcgatccgcccgcctcgcgttcccaaagtgctgggaccacaggcgtgggccaccgcgcccggcgcacatttgggtttttcgtggtcagcaaaagcacgaccaccccaacacataaatctggcgaatcatacgcaaattccctcatgctata

SEQ ID NO: 37 - DNA, 2001 bp, Homo sapiens
tgtgccaggaatttattaatggagaaaccccgcctctactaaaaatacaaaattagccgggcggtggtacatgcctataatcccagctacttgggaggctgaggcaggagaatcacttgaacccaggaggcagaggttgcagtgagccgagactgtgccattgcactccagcttaggcaacaagagcaaaactccgtctcaaaaaacaaaacacaaacaagaaagaaaacccaaaaaacaaaaaacaaacaaaaacccatagatggtacaaaaagtggaagaaaaaggaaaaaaaaacccatgaaactatctgcaatgcagtttcctagaatcaaccacagtaatcttttagcttttctctatctaaatgttatacatattttatatttatattaagtgcatttatatttatattaaaatacagatacaaatttaaaaactctttattattaaattttcaaataaaaagcaacttttcaaataaaagcagcgagaacctccttatacccaacacctggcttcaactattagcaacccatggtcaatatttcctctatttcccacttaatgtctctactgaattatttaatttcacccataaacacttgtgtgcatctctaaaagttaaggactcttttttgaaaaagactttaatacaatttctcatctcaaaaaattagcaataattcctcaacagcatccagatgcaatttttcatgcttttttcagttagtttaaaatcatgagcttttctccatgtctctaaaatgtaattactaatgcttctaatactattccaccaggttgagtgtattataaatgatctgatcaccccttgcctcccactcttccatccccttggacatatttaggctacaacatccccaggaaaatggtttccattgtctgcacgtaagcaaacaggcattattaacctattagctgtccactgctccaccccacccacaggtacccaactagtcctgtgcacctacctggcgtggactgccaagggaacctctggatctgccagtcagccaccctgtcctgctcagactttaccagagacgagtatttccctttccttccctctaagagcattttatttgcagagttagccaatagtatcttgtttcctgtaagggagactatctttagatgctgccacaggcaagctcagatactccagggtggaggccagctgccaacaggagggcctgactttataaacctggaagatctaaacctcagggaagactgcggagctggagggagagaggtgccacttaccccaacctggaggtggggggcagggagcaggccctgcagtcagcaaagcacagcacagtcagctctcattaatggggagccttcaggaagctgggagagctcatggggccctcagcttccctgcccacgagagggagcccagagtactcactggaccacactggggcacacacagctcttctcctcaaagtgcagtcccaggcactgggagcttattaaagacagcacctcaagccctgccctatatctgctgaattagaatctgcattttaatgaaattcttagaagagtgatatgcatactaagtttgagaagctttggtactgaaggcatctagtgagtaccttgcaccttgtagatgctcggtaaatatttttttcctcacctctctgccccaaggcatttagtggctccctatacccatcagtcaaggctgaggacacagctttcacccaggacctggcctatgctcctttcacagtctctccccctgccactcttgccctgcagtggggagcctggtgggctccggcatccagccatgcttctgctccctggcctggccagcttggggcgactgcaacgtgaatctcaactgccactgcctcatcaccttccgatcacttcattacctgcctgacccctggcatatgctatataattctgatccatccctaactagggcctggcccatggtggccttcaatacac

SEQ ID NO: 38 - DNA, 2001 bp, Homo sapiens
ctcatcctgagtttatttcatcttcttattgttttcttctgaatttacatcttttcatcagaacaaaaagaatacaatcagtttttcagaagtttaaaaaattttttcaaatatatagcaaagaatgtcatcatttttatatactctaccaggactcctgaaaccaaacacttaagaaaggttatgtctggaaattttttcaaaatctatatgcatttcagtggtggagaaatagttctgtctggaagcaggaggctgaaattatgaccctgaagatttcttccagcttttgaacttcttattctatgacaactcctggtagatacaggtttcctggtagggattgagcaagaagcatgcacaattactttatgagtcttgataaagctggaatttcaggttgatggttagaaggagttcggctgggtcgtgagaatgcttgttttcctggttactggatatgccatgggctgtatatcttgtgttgtcctgtggtaactgaagccactgttgctgtggaaaatgatggcagtttgaatgagataatttagagtgacctatggaaatctgaggctgtctccttaatgtacaagaccttgtatgtgtggcactgttgtatttgaatggcacacatctctttagttcccttcaactgaaatgacaaaaggtgagaaactgaatcgtaatttgatggcttaatattaaaaaaaaattggcaaaaatgctaaataaggaacaattcagaagaattgctgatcatttcaaaaatattatttgcatagcatttataggtagctaaagaaagagaaaaatggcttaatattttaaaaataatttggtcaatttagcgatctggggattctgcgatccccagaactttgcatccaatttccaaaaaccaaaccttacatctaatgtaaaatgacacaaaaaattctaaatccagtctaattcagtagtatatagcttattcccagtgatgtctcacattcgcaagcccgttacacgttggttttacctgtcaccaaaacctttgtatcactcttataaaaaaccacatcactttctataaaaagtgagtggaagaatctaatattgtaaaactgcacgtgcctcgcgtgacctgagcagaaaattcctagtttgggattttctcacaggcatacctaaactgacctctgctcctagcaggtaaattgctagaaggagaagcaagtgtcagtccaaagaaaacccttgttgtgtgattttttttttctctttaatcgctttttaggtggagtacccgctgcattctctaaagtctttgccaccaccagattaccaacgttttcaccaatttgccacctccctactcctctcctttccaaaagccctcattggaaagaactttctctcgtgacccccctattccatctagttccaatcactcccagcgccccccatctccatcggcttcccctctcccgcccagaatccaggttcccaaaccacttaagagaaaattcacaaccctcgcccccttcccgggcgtccggaggaggcgctggggtgggggtgtctgtgggggacagggtttcctggggtctcctgggtcttcctcgcgcacccccaggaggtgttgcagggggaggggagcgcgggtgggtgggaccgggccgggtgggggaggggtgagggcgaggaggaggaggaggaggaggcggcggcggaggagtggtgtgtgtgcgagcgtgtgtggctgggggaagccattgcctgtttaatagttgctgttgctgcacttccgcttctctcccagcgagagagagacacgagtggccaggcccagccgcagccgcagcagcagccgccgcggcggcacggaggagccagacacaaagagaggtacggggatccccccagcggcccgccgccccccggccgcgccgccgggcctcggggacacccctcgccgcctcccccgactcgcgccgccggggccgccgggccgcagggcac

SEQ ID NO: 39 - DNA, 2001 bp, Homo sapiens
aatagccagcgtgaaagaactgcccacatactagggcttttataaccctgggatttggcctctgtgaaaactgatgacggatgtcgttttcttttttttcttttctgagacagagttttgcttttgttgcccaggctggagcgcaatggcgtgatcgccgctcactgcaacccctgcctcctgggttcaagcgattctcctgcctcagcctcccgagtagctgggattacaagcatgagccaccatgcccggctaattttgtatttttagtagagatggggtttctccatgttgatcaggctggtctcgaacccctaacctcaagtgatccacccacctcggcctcccaaagtgctgggattacaggcgtgagccactgcgcctggcctcatagatgtcgttttcttcatgaataagctagagtagagttcgtttgaaaatacacacacgcaccacacgcacacacacacacgtgtgcacacacacgcatacacccccccacatacaagcatacagatgcacacacacacccatatgcacacacccacgtgtgcgcacacacacacagatgcagacatacatatgcgcatacacacaccaatacacatacacgcacacacagatgcatgtatacacacacacacccataggcacacacacacatgtgcacacacacaaggacaacagcaacacaaacaacatgagccagaagctaccacagtcaggctccttgcttctgagcactcacttgctccaggtaaatatccacgctgcctgttgaggttgtgctgatgtaagcaactgccaagaatggatggacagggtgccagctgataaatgagggtgaccctgctgcatccggggcttctatctggtggtcataatagagggccatgacggaactcaggcctcctcagcgctgaaacctgcagggaaaaaaaaatgaccaagtccaatcagttttaaataaaacaaacaatatgactgtacatggaatgacgaaaaggaagcatcccaaagccaccatcggaagaccatcggcaacttgaaaatgccttgcggactgagcaggcaacctgatgcgaggagatctccaacagggagacatgcagaagagaagcagttagacccggaatcacccaggctgtgacagaaacgaatacaactcatggccatcaaaggaggaaagaaagaaaaggcctgcagcctaactacacacgcaatcctcagtaactttcattccagtcacaataaggacaagttctatttctgtagttccctttaactttttagaccaagtcttgctctgtcacccaggctggagtgcagtggtgtgatcacggctcactgcagcctcaaactcctgggctcaagtgatcctcccacctcagcctctcaagtagctgggactacagacatgcactaccatgcccagctaatttttaagtaatttgtgtagacacaaggttttactatgttgcccaggctggtcttgaacccctggctcaggtaatcctcctgccttggcctcccaaagtgctgggattacagccgtgggccaccactgccttttaaacaacattttcaaacaaaataaatgcaaactgctccagaaaagagtactggtacagaaaaaaaaccagtactgaatttccaataaagcataaaatcaaagactgtcttttacctctgactcacaggtggtagaagacagaagtcttcctctattagagagtcaagaatgcacagccgcagcagctgtgagaagttgagcttgatcacttacgtgaacttaatctctctctttcattcttcctttcagatctggaaaagatatacacatcgtcccttttcaggcgggttagggtgataaataaagtgacggtctggttactgctcacttttttacacctcagtcaatttggggaagttaactggcagagaagaacatacttgtagccgggtttgccaccatttaaaattatacaggctcatgcctgt

SEQ ID NO: 40 - DNA, 2001 bp, Homo sapiens
ctgttgtagacccagccatccaggcatggctccatggccctctgggtgtcattgggcaggctggcattgggcggatgtacaaaacggaggcacctctcaggcttcccatttgggcccatggggagcacccaaggccctgtggaggcattgtggggcgggcgacagtggtggacaggggtggcggctgtgaagatctgcagcaggttgtggttggccatgttgaggatcgggaggcccagtatggctacatgcaggaactggaaatggcccatgcttcccacacggtccaggatctccgagaaggtcatggcactggggcaagacgagccagagctgtgggcagggcataggccagggagaggtatatttgtgcctggtagtgcgtgggtctatggaacgatatgtgggagggtgaaccaggtggatatgcgagcacaggtgtgtggagcagagcatgtgtaagtgtgtaaagatgtggaagctcaggcatgtggagccgcagattcttgtgtggatgtctgaggaagagcccagctgcacggagggctgtgggatcaaaggtggaagtgtgtgtccaggtgtgtggacacaaaggtatctatgggtatgtgctcatttaggctgcagcataaggacacagctgcgtgagctcgggtatgtgttgtgagcgtgtgcacctgcggacacgagcatgtgtgtgctcagctaggctggttgcacatgctgcatgcatggagcagtgcaggacacgtagcagggaccaaggggcccagccttggatgttggctacaaaggacagattagagggtgggtggtgaatgtccccagttgctccaggcagatacacagggttagcagagttcaggttgtctgctgggaaaggcctccccaagctcagagcctcctggcttctggcagttccctggggctgggcccaccatgctggagtatctgtaagagcccgaagcaggtctcagagggcactaggggcttggctagtcgcaagtggagcctggccttgggagatccctgggattcctaggtgcctgggctggggaggccctccatccctcctcacctggtggccgggtctggatccgatggggctggtgatcagtgtagttgaggtccctgctctgtcctaggggccggcagctgctgtagtagggcagctcagctctaacaagctgtgtttgtgcctcccccaggggggcttatataaggcataagctgtttcccgggctaatgtttgactttcttttgcggattaacgtcactggcagggagagtgctgtggacccaccaagcccagctctcctcaaagggctgcctcagcgagggccgctgggggtggggcagcgtggctgagcctagcagggctgctggctggacaccacgggcccaaatgggaagtctgagacgtgcctccgttttgtacatccgcccaacgcgagcagccctggcaggctcaccgaggggagattctgggaaggaatctggcctgacctctgagggttcaaggtccctctcaactgatgttcagaggacttgatgctgaggacaaccaatgggaaaggcactggtgcccgtaagcaggcagcattctcctgtgtgctacctcccctgcctggcacctcccgagtaggggaaggggatttgcctctcaggaagtgttagggcacacccagacagaaaatgtcactttcatctatccctgaggacgcagggctgccatctcctgggcccctggggcagccaggacctccgtgtctgtgtctctgacacaggtgaactgcccccttccatcccaccctcaatggacaggcttctctcctgatcagccgcttccccctggggaccaaatgtagtccctgctgttttattttcactctgaatattattttaattatataataatgtgtgtttcctgcataaaacatgggaaatacagacaagccaaacaaaacaaatcaaaacaaagcaaaatgtccagtctcacct

SEQ ID NO: 41 - DNA, 2001 bp, Homo sapiens
atggcctagctgagtgccagccaaggttgactcattcaaaggcagcaggggctggaaccttggccttttgaggcccagtgcttttcccaataccccactctacgttgcctctgcccatagtaggcagaggaaggcacagggggcaggcctgttgaatgcatcttgggcaccactgttcctgcgctgggtggactgctgagtgcaggggccaggcctccaagcagggaggggaggccgggacagcccctccagcagtaggggtaggggtggaggtagttacaaggtttcagataaagcgctcctccagtccccaggcagcctggattctgtcggcaggagagggtctgccctggaaaggcctcggggcttctctgcaggtctcaacctggctccccctttgaacagaaccttccaaggctgcaggcttgcatcctgcctctcagccaagacctgtctcctcagtcatctgaaaccaccctaccacgccagacgctccctctgccttcttgctattccagctctctcttcttcccacttcctattttctctccattttgttcaaactccatgaacaagaacaacagagctcagacagcgaaagcctccccatcccagctgtatgcaaattcccagcaggctgaaaggaccacccgcccacccaagaccacctcccaggaagccgaggccttgtgtgcagccccagaggagcttgagctgaacggagaatgggttgaggtgggtgggacccatcactgttcttcccttctcattcctgcgcgttccctgaagagtctctggggcctgcttttccccccagcagagacggatgtgtcacatttatggcatcccatttccttcacagagaggctatagatcctgtttcattctggtgtatttaattcactccataggaatcaataaaaactttaaattaaaaaaaaggcaaaatccaattgcttctcattttcttgatccaactggcagtcggcatgtcacctacgttcatgtgtgttctggtgagctggaggtgggagaaggagcttttcattgtacagtgaggcactaactttcctttcccatcatcagagatggggggcttcaaccttcaagtgtccagctctgcatgaagggggtgaccgtctgccctcccttgcagcacccctggccgggctagccttgcctccccatgtgctctccctcggggtcaactttttcttttccttttcttttcttcttttttttttttttttctgagatggagtctcgctctgtctcccaggctagagtgcagtggcgcgatctcggctcactgcaacctctgcctcccaggttcaagcgattcttctgcctcagcttcctgagtagctgggactacaggcatgtgccacctcgcatggctaatttttgtatttttagtagagacagggtgtcaccatattggcctggctggtctcaaactcctgaccttgtgatccgcccacctcagtctcccaaaatgctgggattacaggcatgagccactgcagggccaaatttttcttacctttccagagcaaagccaccatgccaagaggggccctgcgcccatgatgagctctggcttgggttcccggcccaccgtggatgtgccagacccacccacaacagcctctgctttgccaggagttgtacccacctgcttgcctccagcgtctgtgcctcagccctgcctctgctcactgatgccctgggaatcagcgcgggcactcagtgcgaagcccggctgggcactggaaggtggcagagggccctggaccaggctggctctggcttgcaccagcgttctgctttagggcttatgctgaccactccccacccactttctccaagggctacttacggcgtgcacgtggtctgagtgggtctggaggagtctctctgcttgtcttctggtgtccggctgatctgacgggttcttatatacctcccaaccctgggagctgcccttggagcccattaacc

SEQ ID NO: 42 - DNA, 2001 bp, Homo sapiens
ctgaatttttccttctggtgatgagaagcaatgccagaatttctactccagccatcttcacacttgtggagagaaattcaatagtgatacatttgtagtgaggagaaagatgtgttagcttaacaactgatgcagggtctatcttttaaggaagatattgtgtttgtggaggaaaacatcttggaaggtatttagaagtggaaagtaagactttaagggcaagagaaactctgcccatgtcaagagacacaactaaactgctttgtatttggaaatcaataggaatttggagagagaaactaaaaacatgagtaatcaacaaaagatgatttaacctacatgatcggaatctgaagcccaagatttggaaggagagagggaactttaggaacatcaatatctaacatagaatattacatcttgagccttttggcagtgtaaatctctacatcccctataactcagtagtcctttacacatataagagtttctataagtagtttggcataactaagaaaaagtatagtgatccacatctaaaacaatataatgcaaaacataataatacatattggacatttagattatccctaaactgattcacactaagcaggacttaagagctgaaagtgatcataaattctgatgaaatttgaaaattcttgagatacaggttgaactcagtggaaaatacattgaatcaatcttactgatctcaaaaggcagggaaatgctgttgaaatttagtttacataattattttctctttttctggctgtccttaaagctaacacttggcatgatactaaaaacattgttttaagagaaaatctattgtggacaaatacaagctttaggaataaattaaaatgtttagaggaattaacacatctgtgtaaaattttacatgtgtgccaaactggtccccaagctaaaactagtgacatattaaaacccaacatattagctcacacatttacaatgaaatacaacacttttcgtaatactatagtttgtgacagttcaagaagtgatgaattggaaaacatgctatgataggcatgggcaatttagagctcagtctggacaatcggtgatgattaatcttaagaggatttctgaagttcaggcaacaaatgattaaaaatcaacttcaacaatagtcaatgatggctgaatgtaagatatttaaacatgcattggaaaatacaatggaactaattgtagtcatggacattgagagaagacttctggattcagacaaatttttgctcactcaaatccctgtaacattgtttaccttatattacatgtcataaattgactacgttatgaaataggaagggtaatatctatttttaaatgtaataattataaacaatgtataaaatatgtgacacaaagaagactggcagtttatagtgactattgtattattattttgattttagtagttgaacaggaaaaagagagaacagcacagactgaagtatttatatctcaataattaaacttacttaatattttctattatacatttacctaaaaagtacagggctgatacttccatatttatatggaataacagttcttaaaatatataaagcaaaatattattgaaataaagagatgatcgataaacttgtaaaattatcatcagagatgtaaataccacctctacaataggtagaccaaaattagtaaagatacaaaataatttaacaacacaataaacttgaactaattaaaatacaaggtctctatcccaaagaactacaaaataaatatcctttttaagtagaaaaaaaattcactaaaaatatatatttcaagccataagacaagtctgaataaatttcaacaacagaaatcattcatagtacaaaccctgactacaaaggaagaaagttagaaatctcccaattatttgtaaacaatgcactcctaatcaacccaagagtcaaagaagaaatcagaatttgcacaaaacagctgaaagaa

SEQ ID NO: 43 - DNA, 2001 bp, Homo sapiens
ctgtaatcccagcactttgggaggccaaggcaagcagatcacctgaggtcaggagttcgagaccagcctgaccaacacggagaaaccctgtgtctattaaaaatacaaaattagccgggtgtggtggtgcatgcctgtaatcccagctactcgggaggttgaggcaggagaatcacttgaacctgggaggtggaggtttcagtgagctgagattgtgccattgcactccagcctgggcaagaagagcgaaactcagtctcaaaataaataaacaaataaataaataaagtgcacttctctctctctctttctttctttctttttttttcttttttcttttgagatggaatctcgttctgtcactcaggctggagtgcagtggcgcaaactcggctcactgcaagctccacctcccgtgttcacgccattctcctgcctcaacctccggagtagctgggagtacaggtgcccgccacctcgcccggctaattttttgtatttttagtagagacggggtttcaccgtgttagccaggatggtcttgatcttctgaccttgtgatccacccgtcttggcatcccaaagtgctgggattacaggcatgagccaccgcgcccggccaagtgcacttctttttcagtaaaaacagttggctgacttcaaggacatgtgaattaagtagcattacgtatagttcctagacaggggaaaaagccaaactggtggaaccactggataatgactgaggtttgggaaactgaggctggatgagaaggttccagaggcctaccctaaatcaggaagcagctgcgcttggtagaaatagtgtctgtttggtctgtttggtaggaggcctgcctcccattgagccaacaataatcctgttgacataaccctgttgatcaaagcgatcccctgactgctctgggccagttcctttggctcctggtggaatgcactcagagaatccggatccggtcagtataaaaggtctggtcccgcagtgggggagccacatcctggaagagtggcctaggacagctcctctcctgccagagctaggcaggcgccgaagtagccgcatggccccgtcagaagaccccagggactggagagccaacctcaaaggcaccatccgtgagacaggcctggagaccagctccggtaagaggcggcaaagggaccccagccccacagaaacaccagctgggggccaggtgcggtgcctcatgcctgtaatcccagcactttgggaggccgaagcgggcagatcatgaggtcaggagttcgagaccagcttggccaacatagtgaaaccccatatctgctaaaaatacaaaaaattagctgggtgtggtggcgggtgcctataatcccagctactcaggaggttgaggcaggagaatcgcttgaacccaggaggcggaggttgcagtgagctgagatcgcaccactgcactccagcctgagcaacagtgtgagactccatctcaaaaaaaaaaaaaaaaaaaaaaaagcaagaccagctgggggtgggtgggggtctgatggcaaacatgctacctcccttgggttgctgggattctccagccaagggcgggctcgttttatttcctgtgctagccaacctactagaacataagcttccttctgagcccacgtcgtgtttcacagcacgtagctaagagaatggatgtgtatgttatttgggtaactcaaaaagttacctttttaagagcacaggctttggattcagatacaattgctcagacacctgcttcattgataagtctattggctggtttgtccatccctctaccctgtgctgggctctgagcggggggattgccagggcaaatgccaccaggtactgatgggattctctgaggctgtggacaagattatctggggagggggagagtagcaagatgctccctgagacccaccaacacttgtgcaggctgagcctcagtttcctcacctggaaagatggccagggct

SEQ ID NO: 44 - DNA, 2001 bp, Homo sapiens
taaagaataaaagtgaatgaaaaaatggaagcattgcaccgtgcttttgatttacatttccctgatcatttagtacaaccaccatggaaaacagtgtggagattccttaaagaactaaaagtagaactaccatttgatccagcaatcccactactgggtatctacccagaggaaaagaagttgttatacgaaaaacatacttgcacacacatgtttatggcagcacaattcacaactgcaaaaatatagaaccagcccaaatgcccgtcaatcaatgagtggataaagaaactatgatacatatatatgatggaatactactcacccataaaaaggaatgaattaatgatgttcgcatcagcctggatgggattggagacaattattctaagtgaagtaactcaggaatggaaaaccaaacattgtatgttctaactcacagtgggagctaaattacaaagatgcaaagacataggaatgatctaatggactttagggactcaggggaaagggtgggaggggggtgaggaataaaagactaaaaaatgggttcaatgtgtacactgctcgggtgatgggtgcaccaaaatctcacaaatcaccactaaataacttaaccatgtaatcaaataccacctattccccaaaaacctgtggaaataatttttttttaaatggaagcatccaaaatgtaatttgtttccacattcttgtaagcctcattatgtatcccagggacagttaatatcgtcatacgaattcacaggtgaggaaatggaatcacagagagattatgtactttgcataaggagagcgagggtgaggcaggatttgcagtctagaagcctgggtctggagttctccactttgcactatatcatttctgtaaacttcttacttaagactgtgtggaaatgtagagtgagacagaactaaagctcactattcttagtccacaggggagtagtgactacccagggcttggtaagtgctcagtaacgtttgttgaaagatgaatcaatatttcaatgctggggcaaagcagtgaaaaactggggaatgtccaggaaccagccatcaggataaggcaggactagaatgaatgcaacagtcctcacaggtccaccaagtcaacaacagaaaaatccaaagctaaggtggctcttgtgtgatcaaggacctcttgtacaccgacctgttaggctgtggcacagtcaacatgggagttgtagacaagctacttgagagtggttaatggtagcagtcaggcatttggagctggaggcaaggctgattctcctcaactagtatcatattgagcggggacttaaagcctcagaagtctgagcagaaaagctgagaccaggctccttgggacaggctgttcatatgcatgattgagtttcagtgatgagtcccagaaggaaaaagtttatgcatgttttccttaaaaatatattcacttatatttagagaatccttcagatatctaacctaaatacgtctgtaacatttactctcattctttttatgacattattagtataataatatgaataattcccagtggacttacttccacacttccttcaaggattctccattttatttcataattgaagactgttaataaatcactctctagctaagaacaattcaatgctaaaacacttagcttatctgaccttcacattaattatttttatgacagcagcaggaactttcagctgcttttcccccttccttatgaagccatgtggtcccaagcagacagagttcccttgctacacttaagggtgggcttctcagacctgagcatatcttaaaagctttcccttgactgtgccaatccaaactccaagtttcctcagcagatttaaggggttttaaatctacttgagcatgagtgttatgagaagactttgaaattcttacaaaaactgaaagtgaaatgaggaagacagattgagcaatccaatcggagggtaaatgccagcaaac

SEQ ID NO: 45 - DNA, 2001 bp, Homo sapiens
gtccagtagacatcaatcaaactggactcagtttgcacacacctggagctcaagagtctccagggggaaaacagagacacaaagtcagacagagagagagccagagaaatttcctgcaccgtgaagatagtcagaggcagggaagaaactccttagcactagttagagtgatcagaaaccaagaggacctgatcgctgtacctgccaggtctcagtttctgtctccttccaactgaccacctcttcctctgagactcaccagttctgcatctcttgctcctccttctgtttctccgaccacttccacctgtggctgtcacagaagggcggatgaaggaggggacactggagatagactcagcatctgcaggcttccaaagagaggggctaggagatccaccaacacaccagcacaaatacaccagcacacacagatacacacaattggttcatgtattgctaggttacagtttgctatgctacaaaggcagtaggccaaatttgattgaattgaataattccttattttcatcagcttctcctttttttttttttttttttttttttgagatggagtattgctgtgtcacccaggctggagtgcagtggtgtaatcttggctcactgcagcctccacctcccaggttcaagtgattctcttgcctcagcctcccgagtagctgggattaaaagtacccaccatcacgcccggttaatttttgtgtttttagtacagatggggttttgccatgtgggccaggatggtctcgaactcttgacctcaattgatctgcccccctcagcttcccaacgtgctgggattacaggtgtgagccaccgcacccagccagcctctcagttttgaacatgcactaccaccacctccacaacacacaaatgtaaatgcactttcgtatataaaactgtataaatacaaggaagctcatacacatgcaaggatacacacataagcacccccagattcaaccacagaaatatacgccagtacatttgcataaattcaaacacccctttacatgtaaaaatcatataagcacatacagggatgcaagcaggcatggacaaatgcatgcaagcacagacaaacagacaaagctaagtaaaaaagtgcaagctcacctatgcttacaaaaatagacatacatatacccacaaacccacacacccacacattcacttgctcacctggactttgatatctctaccactgtatccctgccaatatctacagagtgggtaaagggataggcatcaggtcactgggttgcccaagcaggaagtctgggttccctaacaactttttctaagctaatgctcctggatgatgatgaaaaaggaggtggggaatggatgaaattttataacagggtgcagaggcagggtcaggataaaaggcccagttggaggctgcagcagggtgcagggcagtcagaccaggaccatggaactcagcgtcctcctcttccttgcactcctcacaggactcttgctactcctggttcagcgccaccctaacacccatgaccgcctcccaccagggccccgccctctgccccttttgggaaaccttctgcagatggatagaagaggcctactcaaatcctttctgagggtaagacacagacgaatggggtctgagggtgagctgcttcttgccttggtacttggggaagcttcaccaaacagaatgaggcagacttccagagtcaggggtggcacgggcatggttggtgagtacggagcatggtgaagcatgatgggtggtattattaggagaaaagcatcaaattaaatttagcagagtttatttgagcaaagaagtgactcatgagttggacagctccctaaaccaggaaagacaccacacggcagtatggtcaagtggtatttacaggcagaaaaaggaggtgacatacagaaacagcctgattggccacagatcacagcttgccttacttggtcacaatctgagcagtttgc

SEQ ID NO: 46 - DNA, 2001 bp, Homo sapiens
agtaatctagaccttcctcttcctatcacagtctcaaggattgtgcccccaaagcagatgcaaagccctgtctaccccttctccttcccctgaggtagagctttatcgaaggtttctcttggtaaacgttagctttctgaactaagggccgtcttttttttttttcccgcttaagcccattgcataacaatctcttgttttaatcttttgcccaacactgcttttcttactgtcagaggttactacaagtgtttgaacaggtaacttctacctttgaacttctgttgtgacagattcaaaggttccaggggagtgtgttggccactctcaggacagtacacagtagcttcgggtgtgtcccagtcagtcctaggagctgtggaaagagtagaagtgcctgaatgtggtgctgaatcaatacagccagctgtgaggggagcacttcctggacccaggaagggagagtcttcttccaaggtaagctgcaatgatactacctgcactatattgaatatatctgtgagatagtagggaggggattggtgtggggaaagaaagtgggatttgttatgctcaacctaaggctctttaataacatgaaattacatttagttgaatagtaatctcagctgacttgtgtgggaggcaaagaaaaaaatcagttggaaaaggggtggagatatatgagctatccaaatgctagcaggggatggaattggaagggaacactgattttggtaagtgtactaaagcacaccaagccagaatgtgggtctccttcatcttccattaagaaatttctgtctcagtgtagttagaaggatcgtgcgtcagctgctgagggcagagagtcccatttggcagacagagaggaagggaatcagcagatattcgattaggtgctggggactgttctgggtagattatgtatgttacttcattggtgtatctcatcctataaagcaggtggtatccttcccatttcacagaggcatagtgcgtctccaggctgttaaacagcatggcctaagctacatagctccaaggtggtgtggctagaatttgagacttgattggtcagtttccacagcctgcccaataatgcagaattgttgtgggttgccaaagctttcagttaccccctgaaatcctgagggtgtcactcatgtgcacagaggattgataggaatacaactctgatgggcctttagaatgactcaaccttcctgaacttgagcatttttactggaggaggtcaataaaaaaaaaagaaataaaacctcatcttcccctggccctgagccctttggtgaaggaagacagcacccggtgaagggctcagaggtattagtgtggctgagattcgacacccactctgccatcctctgctctcctctagaggaagttgcctcttggggagctgagttcctgaaactaagcaaatgacaagtgctcctccagccgtgagatgagccagaggatggaatgccacaggccccacttcgtctcccagcccttcatctttcttccatgtccaaaggcggctccccagaggccaagggctcagctcttgtgttctctgagactgtggcacagctcaccctggcttccttagtaggaagcttttaagaactgctctgtagaaagaacaaaccctaatctagctccagcaaagagccaggatcatgctgaacttcctgtgagccttcgtgggggatcgaggctggagaaagtgctgcttcattctgaggtatctgtgaggtggggaaggggcaggaaaatttgtcatttcggcttcctaatctgagaatggtagctccagggcccccctgtagccacagccttaggcggatcaaaaatgagactaatacaggtcctggtaatactgggggtgaccaaggcgaaagaatcgatttttaagttaagttttgaggtgtgactgtatcagagggaaagaaaccatgccaattgaacgactcacagccctgtcgttcctgactt

SEQ ID NO: 47 - DNA, 2001 bp, Homo sapiens
acaaattacaccattccattcaatgcagcaatatttcttcatgcttatggtgtgctgtatctgatgataaaagcacaaataagaggatttacatttctatcttggaaatacacaatatctataggcaaagagacaaaagaaaaggcatttcatttgaaaggcataggtcagtggtgagatctacatattctcttgatactctggaatagattgggtggggaaggcaagatacctgttcttaggaagtcactatctcactctggttctatattatctgtagttctacactgtcaaagaggacattgaacctggtgatatggtttggctctgtgtccctactcaaatctcatcacacattgtgatccccataatctccacgtgttgagggagagacctggtgggaggtgattggatcatggaggaagtttcccccatgctgttcttgtgatagtgagtgaattctcatgagatctgatggttttataagtgtttggcagttcctccttttctctctctctctcctgccgcctaatgaagaaggtacttgtttctccttctccttccgccatgattgtaagtttcctgaggcttccccagccatgcggaactgtgagtcaattaaaactctttcctttataaatgacacagtctctggtattctttgtagcagtgtgaaaatggactagtacacctggtgacatcaagacccccagcatcattcaagatttattgtctcttgaacaatatgaatgactgactaaagtgcctcaaattctgtagtttgcatgtgggatgagtcaaaagggggtgagaatgttaacaaggcacatgacccatgggaatttgctgggcatatcctcagtcacagagcaatgaggggagagtacaggcaaaaggtgtaagcagaaccagccacaatctgtggagaagaatgagatgggatgatgtttgcttggctgctaacttgataataagaatgttagcctttggcttcattttacgtgaatacagggagttccaccacaaagtgggtaggattatgtcccctaaaaatatagtttaagtcctaatccttctacctgtgaatatgaccttgtttggaaatagggtctttgcagatgtggtcaaattaagatgaggttgtactggcttaggatgggctctaattcagtgactgctgtctttaaaagaagagggaaatttggatacatacatggacaataaagagaaggatgtgaagatggaggcagagacttgagtgatttgtctacaagtcaaggaatgtcaaagtttgctggcaaccaccacaagctgagagagaggcatgaaacagattttccttgagagtcttgaaatgaaccaaatctgctgatatcttgattttggacgtttgtcccccagaaccatgggagaataaactcctgctatttaaaccactcagtctgtggtaatttcttatggcagccccaggaaacgaattcacactactaaatgaaaatagatctctgtctatcaatatcaacaccaatatttatatacagggatgataatgaccttagttttccagttcttaatagcatactgaataattgtgcatgattgtatttcaggtatgtaaaataaaattgtttgttcacttattttcttccaatcaccattttctctttctcctttttttttggttttagatcgttataagttttacaatggactctcttgttacagcaaacaccaaattttgctttgatctttttcaagagataggcaaagatgatcgtcataaaaacatatttttctctcccctgagcctctcagctgcccttggtatggtacgcttgggtgctagaagtgacagtgcacatcagattgatgaggtacgtgtccactagggtgctacacagggtcctaaactctgggtccacactcaaagtcagaccctaatcccacttaggcaagccaaggggcttattgcaccctgggcttggtcagaacccatccctg

SEQ ID NO: 48 - DNA, 2001 bp, Homo sapiens
tttattctccccaaccaggtggaaatgtaccctgtttaggcccaggacctaccttagcaaccttgattggctcacaagccaggttgcttaagacttgtttccccagcagctggtgtccctacttcctgcctagcccttttccttttgctttgtctgtgcttgggaaatacatgttatactctgtagagcagatttgctggcagtaagaacagagatgtgtagagcccagcgtgctggagggctctctgcatctggctgtgagtggcccctggactgggaccgtgggaggacagctttccatagtttaacataaatgaaccacaccataaggacaatctgctctcatttcactcaactaggatttagtgagtactgttaaagcacagtttgcactggagtaattggagaatggagtttgcccttcacaatctgtgcacaatctgttagcagctaaaaacaaatgttagcagttataattaccatcaatttgtctccaggcttggatcacattccaggtgttagaagagatggtccatttccttttttttctcctttaagtcccatgccctgtttcaagtagcttcagaaggcaattgtgctgtaaagattcaccttgtaatgggttcaggactctttcacagtttgtgtctattcgttcatttgaactttgctgttccaagttagaaagcccttgctgaggtgtggctacctctaagaacactgttgattgggtgccatggtaacgtttgcaaagtagctactgctaggatacagctctgacacttattactttatttgcatatcaagccacctgttccaaagatgaaaatatacaggactgcatatcccagtctgggtaaaaggataatagctgactgttgaagagaacatgttgcagtttttctcctgctagctgaagaatcaagttctgagggcctgctcctcagcacctttctcctggtctagagtaggtgccaggcctggcggttctgttgcctgcgtgggtgccagggcttctccttgggaggcttgccctctgtgagagggagcagagagttgacttcctccaagaggcccatagtccctgaagggggtgtttttgattctgtctaaacctaggctatgtgttagcggtttccctcctcttagaaattgcctggaagactcttgctattggtatgtatgtggttccctccagtttaagacaagatttgttccaggatcggttgtagccccttctctgtggctaacggaagagccacttgtccttctccattcttttccttagtctcccttgacccccacatcatctcctagacacaaggcacattgatagtcacaattcactgtcaatcactctggattattccctcattcccctttcccaggccacctgaaggagtgagtggggattgctattgtgtacattccagtaataactattggctgtaagcagacatactctaaaaataactcagggatactgagcacccagagggcaccttctgtcttctcgaagcttcgccacgcctgtttccactgtcagacttccagtctgcaccacagcacacatcctttctcatccagagtgctcctactagacgagatgtttttgtctcctaagggcctcaggtgtattcatgaactcctcagctggttattttcagtttcatcttccttggtgcttgctctggcacatggtcactgctgcatcctgtcccttccccacagtttgccatgggtcatcccttttttctcattgcttgcatcttctaactgaatgaacttgtaaatatggaactgaaagtcaatcctcatagctcatcttttatttaatcctaataaatgacaatctattgcaaacattactttaaaaatattacttatcaaaagctgggtgaaaggataatagctgactgttgaagagaacatgttggggtttttctcccgctagctgaagaatcaagttctgagggcctgctcagttctgtaagaaagtat

SEQ ID NO: 49 - DNA, 2001 bp, Homo sapiens
ttaggctcatgtgcttccccctgctccccgccccctgaccttggacccccattcaagggcatccctgtcttccttgactgacggttaacactcccaagtgcatggcgctgggctgcccccatcttgagttctgaacagcaaagcctaatctcagagctgctgtgggactgtgactcccaaacctggtggcaggtgctgccttcagcccccacctgagtcagggaccgccacacggccccctgccccaagccttgggttaccctgtagatactgccctggcttatgtgttcactggctcctgtgccccaacccaaaatgccgccaccattgccttcctccaagaggacccccagtatctactgctgcttctcagagccaggagctctgagcaggagcagtcaagaggggttgccggtctgccctcctctgctcttgtgctacactacagagggataggcaaagggagctaacagcgtcgctcagagacaaagaacacagaagcaagatggtttccttggccagcagggagctctggccctgccttctaggctccctgggagtcctgcgcgtcaggcaccctccttctcttccgctgcaggcattgctgactttgagcactcaccctggctctctgatcccagggccaccccttcccctgggtgcgctctccctcccacactctgctgggcatcctgtgtgaaaacatcatcccttaggctctgccctggggctcctaaggaacctcacggaagattctgctccttgctcacagcctccaaatggccaggaaaaagtttcacctctcacaacagttttgcaggagagatgccattctgtgggacaaaatgctgtgggagtattttaggaatgttctaccaacaaaccaactgcacaggcaagaaatggttttctccagctccaagacctcgctttctccttaaacaaaagccccaggttctctgcctgggatcaacctacatctgaagttctgggctgcagacgcttaccttgttcagtcctcgtggagctgggctggcctgtgcctcgccgtagactgcagactgtgaggggcagagccgaggggaggctatgaaaactgtgtccagcagcaactggctggtaaaaatagaacagacccactgccccctcctgctgcccctcctccctcccacccttcacacacacacacacacacacacacacacacacacacacacacacctcacagggcagatgcgtgctgcaatgcacacaacaggcacataggcggaaagccatgtgaatttgtggctcaggagcagaggcaaattctgtgcacataggtgccaccaactgagtgaaaccagcacccacctggggagactcgagctgcagaaagggaaagaatgagcagccagaccaggtgtggggactgagcagagatgggactgggagggggaatgagggagagcaaagggccacagtgaaggcccagaggggcaagtagcagctgggggaagaggaccattgggctaaccctgcatctcttataaggcaatatggaaatgaggcccctgaacccatgccccaaaagcccaagagggctaaagactcagctttcagggatttttcagtggctctggtgcacgtgggatacgagggtgcctggctgtggggagtgatgtgagcagtgttgcaggctgaccccagtgagtgtttccagggctgagcagaatggcagtggcagcagggcacacctttatgctcagggaggtctgagggtctgcagacactctgcatccccctccagatgggtttcctggaatctagatttcccaggttccaaaggacacccgagtctcatgcctggaactcagtgagactaattcacctctcctctgccctaatcttcatctccagccagaagccaacagatcccaggggactggagccacaggggctgcacctgtttaccgggtatttttaggatggttgatgaacacataatacccaccctatagt

SEQ ID NO: 50 - DNA, 2001 bp, Homo sapiens
ggttccttaactttgcctaggtttttcttcctgtctccagtgacagagagaaatcacccctggggttcccaaaggtgcatccccgtcatcatcttcactcactgcatcttgaagcatatggatgaaacccagcaggcttatcgcatagcttgtggtctgccctggggagccaaggaccaagagccagagagatgagaatcaagataaaaataggtgaactaccacctaccaagtagatgactacataggaatggatgtgtgggcaagtggagccgtctgtgtgtgtgtgtgtgtgtgtgtgtgtgtgtgtgtgtgtgtgtattgaagacaagggcaggggttccagacctccctgcatttgagccccatgccctctcttagcatacacacagcctcccaggtacaaacagtgccctgtttggaaagtaaacatgccatagcaatcctggcacctctgtgaagtgacttagatgcccacatgcaccacctacaatgctacctgagtgatgaatgtctctgggatgtgagtgtcagctgagcacgtctgttgggaaatgggcccctcccaagagcctgaacctgaatacttcttataaaaaggttctctggctggacgctgctcaatccactgcctagcaggtggcccattccagttggagaacgtagtgagtctttcagtggagccagggtctggtaagtctcctagcagggtctgggggagagggtgggatgagttgagggcacaggggaggagcaaacagaacacaggcagcagatagatgtggaaggggtgtcccgcaaggcacctacgcttctagtgagccaacctcttaaggtactaatgaggacataaaggcaacataggaggtggtttttgcaagggtacacagcaagtccgcagaagagctgtgactagaacccaggtctttcagctccctggatggggttatttatattggcacaactctcctttcccacatggttgtggggcatgagaggcgtaggcaagaaagaatgatcaggacatgcttctggcctgtggattatttagacccagcaggtgggaccgcctgttggacttgggtgttttctaactgatgggggtgtagtaactaggaagatacagtcgaaggtcggtaggaaagtaaaatgagggacaagaaaaaagccaagtggagattttggactggatcatacagataagttgccgcagtgtgctgtttattttccactgtggagaacttgagcagtcttcttctagtcagagaaaatggaaatgcattgattcattgcatggattgtgtcctgctctgagcatgtttaactcactggaaacacctgcttggatttgcttctttgcgtccgctctttctgggtgcttttccctccttgtcccaggccaggctgacttgtacactaagccgaatctcaaaagcccagacagagttccattggtggaaagaaaaagccggctcgcctttgaaaggctgcctttcttccaggtttgtcgtgaggagctccgcgatgtcctctcaacagagcgccgtttccgccaaaggcttttccaaggggtcgtcccagggccccgctccgtgtcccgccccggcgcccaccccggcgcccgcctcctcctcctcctgctgcggctccggcaggggctgctgcggcgactcaggctgctgcggctccagctccaccagttgctgctgcttcccaaggagacgccgccgacagcggagtagtggttgctgctgctgcgggggcggcagccagaggtcccagcgctccaacaaccggagctcaggatgctgctccggctgctgagaggcccgcaacccccagcgctgcgctagagaaacccgcccagcccagagcgggcccgccccgctgcggctcccacgcggggctgggcctcggagtttgccccgtaaagcgaattgcactttgatgttcagaaacccactttgttctcagccacgcaaaactccctgaccccgatgt

SEQ ID NO: 51 - DNA, 2001 bp, Homo sapiens
gcttccgcagagtggggaggggccgccagtgcgcgccacttttcgcctgtccgggcactgcgccagcgcggggcggcccggaggcggggtcgcgtctctgcgcctgcgcggcgggaagccagtggggcgttgtttcgtccgatatccgcgtttcagtctccgcccatacccctccgggttaggcggctgtagcggagctcgaaaagagtggcgcagggtcgcgcggccccgcctccttccccgcccagcgaagctctctgaccacccctcttttctagagttctgcctcgcttcccggcgcggtcgcagccctcagcccacttaggataatggcgacagctgagggtgagtaacagggatccgggcccttcacggtctccctgacccccaggccgtgagctcgcggccacgtgcgagccgcgaaactctgtcccgcctccctgcagcagcgcctctaggcagccgcgcgggcgcacacgtggcccaggcgctcccgggcctccccgaactttactgcctcggggatggggagaaggagcaaagtagaggagaacgagaaatggccaaggggtggaaaggtgcctggaagtattatttataatcaatagctattgcaaaatacaagcaaatcagtcttgattcgtcaaaacctgagtcttgctttgcccgttaggttaatgacctttctaagaaggttaataattgaaacttttgaaagacctgtctagaggccgggcctggtggctcacgcctgtaatcccagcactttgggaggccaaggcgggcggatcacctgaggtcagtagtacgagaccagcctggccaacatggcgaaaccccgtctcttactaaaaatacaaaaaattagctggacgtggtggcgcgcgcctgtaatccccagctactcaggaggctgaggtaggagaatcgcttgaacccggaggcggaggttgcagtgagccgagatcgcgccattgcactccagcctgggcgacaaagcgagactccgtctcaaaaaagaaaaagaaaaacctgtctagggggcgtgtgtgtaagtagtctttgatgccatggtgtgtgtgtgtgtgtgcacgcgcgcgtgaagtagtctttgatgccacgatagcttggggtgtgtgtacgtgcgtgtgcgtgtttaagtagtctttgatgtcatgggtgtgtatgtgtgtgcgcgcgcgtgttgaagtagtctgattccatgatggctggggaggggggctgtttttaggtagtctttgatgccatgatggtgtgtgtgtgtgtgtgtgtgtgtgtgtgtgtgtgtgtgtgttcgttccagtagtctttgatgccacgatggctttgaagaactccaaaacctttaggcaaaaaagttgcactatcaggaaagattgttcttgctatttatgctggaaaaaaacaaatgctggttttttccacacacccagttgctatatgtatagtgaggcaaattgcttttgtggctgctgggtacattcttttccacatagaaagtaactgaattcagattttaaaaagctgtagactcaggccgggcgctgtggctcacgcctgtaatccccagcactttgggagtcccaggcgggcggatcacgaggtcaggagatcgagatcatcctgaccaacatggtgaaaccccggctctactaaaaatgcaaaaatttgctggacgtggtggcgtatgcctgtaatcctagctactcgggaacctgaggcaggagaatcacttgaaccggggagtcagaggctgcagtgagccgggatcgcgccactgcactccagcctggcgacagagagagactctgtctcaaaaaaaaaaaaaaaaaaaaaaaaaagctgtagactcagcaaaaaagattcagatgatattgagtatcaactccctagaatcaaggcaaggaccagaatttttcttgcaatcatggtacctgtacctctaaaatggcaaatctctaaaatctttgctcttccact

SEQ ID NO: 52 - DNA, 2001 bp, Homo sapiens
accctctcaactctgggtcagggtggctggtccccactgctgtgtggacatgcaagtgagggagtctctgggtcagctggaatcatgaagacgagagttcagggagggtgggagtggtggagcctcagcagagaagttgacggtcattgatgttgaggaagtagattgtgtggagggaggagaatggcatggtgtttgcgctccccaccagcctcacttacacttgtggacactgcggccggtgtcctgaggacgtgtgttgggaattcagccacggggaatggaggtcccttggttcatatagtcacaggctctgtattctaaccccagctttgtaattttccagctgtgcaaccctgggcagtggcttagcatctctgagcttgcctccagatccggatctgtatgatggggttattaatgcttcataaagtaggtgtgaggagggattgaggtactattgggagaaactttgtaactaaaaggcaattgtatcatttgcattattaccagtgacatgagacatagtcacacaaaaattgaaatgtcatttgttttaaaaccagaaattgcccaacttagggccctcctgtctctcactgggtgccctgcaggggaccaagagccagagcaagactgagaacctggggcagaagcgcagggaggaaggaggtggggcctgcttaccaaccaggcctcggccccttcccctttcaatcccaggcccctgcatggcagggagaggccagttccaccatggcaggttctttctaggctggtgtgcagggtgggggcctccaaggagcaggggcatggggaaaggggctgcaggcgctgggcctaggaagagaggctcttatcttgtgggcaggaagtgtgaggtgttgagcagctagggaaccctggggaggcggcggggagcaggaggggtgtgtgttgtggtgaggaggagcggaagtcggaagctccagccgtcacagccacattcactgggcaagccgactgtgagccaggaagtgctcttggggagcccaggccaagccatccattcttgggtcctttggaggtgagctaagtgggtctgcctaggttggggctggtggaacctgtgggagcagggaatgtggagagtcacatgtgggtccttgtggcaggggctggggcaggtgggcaggggagaggcgagggctctggcagtgccgtgtgcacctgccatgagcctgggctaggctaggcccctccttcccccttcctgctctgtgtcccgtccccctcctccctcagcctacccctcctcatctgacccttctctgattccctgccctggccccacggacctctgttgtttcccttccctggctccccgcagagcctctggaggtctgcaatagtgaagggtgaaggaagctccttgctaatcctcttgtgagtgtcactgcgtccccagtgactgctctggctgagtacccagtgggggcttttgtggccaaaggtcccaagtgattctcgaccctagactctggagagagggagtctggaaggcctgggtttggggtgggggtggctctgggtgtggttgctgcaggagctcagagtcaacttggccatcagggggctcccagcttcatcttgccttccaggaccatcctggagaagctcgaggagaggtaaggggaccctccttactggtttagctctggcttaggcctgttgtcctgatgggcaacaggaaccactgaactctcaaagtgtgcctgtgtaggaaactaaggcccactgtagtcattattgttatagtgaaggtagctgcatggagggaaagtcccctgacactgccattccccccttattccagggcctaaaattccacagttgaagtcactcctctgctagaatgcagggaagggtggcctgtcattagctgatgataaatactcctccgttcattggaccttggaccagctttaaaaagtggtccccaagcaggcctgtctcccagct

SEQ ID NO: 53 - DNA, 2001 bp, Homo sapiens
cctcacccaccccagcctcaggatgtcctggggcccctgctcacgccatcatgatgacactgaagtccagccagttccaggggtcccggaggaatgtgaagtcgtcgacacagaagcctcgggccagtatcttgatgagggactcaaaggtgtagatccctgtgaaggtgtacctgggggggagagggccggccgggacaggcatgtcacctgggtaggggtcagtgtggcaacgacagacccccagaagggagggacacagaaatgaaatatgccactcaggtggctaaggacactagcgagtgatagcatggccaccccagggactcaaggtgtggatgagggtcacaatgacagtgtgtctccctgaaagacaagagcagcaccacacagaggtgcaaacacctgagatgggctgtgtcccagggctggggagctcagggagcagggagacttactccacattcttggaccagggaggcgggtcactcatggtcatgaatacgcagttggtcaagatggtgatcatgatgaacatgctgaacagcgtggggcagggtcaaggaaagtgaggaagcagctaggatgggaggtgatagagggtctcctgggccacagcaccccatctcgcccatccctaacccctcccgcctctcccatcttgggcaggatatgcatggatgagcaccttgatggccccgcgcctgactacgctgaaggggctcagcaggtagagagcaggtgtggcggagaagcggaagatggccttgcccttgttgagtacgatgaaggtctaaggtgggagagaggctgtgagacccagggacagacagacaggcagacagatggatggatggcagacagacagaggaagccttcccaggcccagaccttcttattgctgtagtagggatccaggtcctccagggggatgccgatgacctccggcggggggtctccgtagatcatgggtaggttcttgccagcctccaagtcacttcgtggcttccgttcgggctcctcaatctccatctgcttattccgctgcagccgggcctcctcctccaccgcccgctgttctatggctgccagtgactcccgggtgaaggggcgcaagcactcagggcccagaggcaccagggtgcacagagatggtctggccatcctcgcatcctgggctcagagaccagaagggtggtgggtggcctggggagctgcagtgcgcagccccggggtgctgggcggccgcccctccctgggcgggccgtccactcctcacctcctgcttgcccagtggccgagccggggaggtgggcgtgcagactctatcaggactgggagatgtgcctgcaaggtgctgctgctgcacaggtgctgggcacagagcatcacccctggcccccccaccacactttccccagaagtggcagcggctgccctcaggaccgacggcaaaccaaccagattctttgtggccttgggacttggggtcaacaccccagtgccttcgactgcctcctgggcccaagctccgggaactgtgtggcttcagcctaatgggacagagagagaaccacagctggctgtgctcaggcgacctctggataggccaggatgaggggcctggttggggggttagatcaaagtgggactggcacagggacttcaccctgagggccgacgtggatcctgatattggccacctggagatgtaggtggagaccatgaaggtggcagccaggctctgtgcaaaaaggttgctgtgattgattagtgatgtctgccaggggcgcaaggaggaagaccgtggtgcctctgcctgtgtttgccatccctggcctggagggaagggaaaagaaagatgggggtgggtgtggattcctctttcctgatctttggggtacttggtgttgggctgggagagggaagaagaaagggccctgggcccggcaggggtctcctagcagttccctcccacagccctgggggctctagcccact

SEQ ID NO: 54 - DNA, 2001 bp, Homo sapiens
tgggactcaagccctgccattcagattcttttgttccacagggtattcccttgatgtgatgttcttccccttcctctaggaatgggacttcctggaagccagactacagtgctcttctgggtctagccacccagtgggcctaccagactctgggctggtgctagggaatgtctgcaagggatccagtggtgttacctgtcttcaagactcccagcagtgaataccagcaccatctctgacagaagtgggaggggagtgatgcaaactctgcaagattccttggttgtagatagacttagagtattgactttctcaaatgctggttataattatagtgaacttatcacgtggacagacgcaggacttctggttagccagggtgttgtaagcagtggtgatagctgagaatatgcagccgttttctccttcctgggtacagtgctattctgcctagaggtgagtttttgcctgtctcacagagtaggctgcagcctgccacttctttcaaaagttctgtggattctttcagctttcctgttcagttcctgcattcgttcctggagaaaaaaaaattatagtgtgaatctctacacgctattctgtccttccagatgggagaggcatgctaatgctgcctccaatctgttgtcttggggggaaagaaagcaatttgtttttgtttttttttttaatggatttgctgggtttagtaacccagaggtcattgggaaccttggcgaaagttatttcagtggagcagtaggaaggaagccagttgcagtgtctttgttgagcagcagaggggaggtgaggaagtggagagagcagctgtggtggctgaagggtgctaggacagagggagcatgataaagccataagtaagatgtggtagaagggaaagagccaggacagaggggagagaaagcaggatcctcaaggaccagagaggcagtgggtcccacagtcaggtgcggggattcaacagaaacagaaggagggacgcttctcactctgaccccggaaagaaggaagaaactgcgtgcgagttcagacaagtccgttggcgggggctgggatgcctgcatcttccttgttaaaaaaggaagtgcaggttcaaaacattcagagacagaaggtggatagacaaatctccaccttcagactggtaggctcctccagaagccatcagacaggaagatgtgaaaatccccagcactcatcccagaatcactaagtggcacctgtcctgggccaaagtcccaggacagacctcattgttcctctgtgggaatacctccccaggagggcatcctggatttcccccttgcaacccaggtcagaagtttcatcgtcaaggttgtttcatcttttttttcctgtctaacagctctgactaccacccaaccttgaggcacagtgaagacatcggtggccactccaataacagcaggtaggtgacttgttcagtctgagatgctggcaacatatttctaatggaagagatctggaaacttcagcctcctctgaggcccctgagaactggggtgatgaagtgtagaagcttagagctaaagacaactaagtcttcacctagacagtgttgcacaaaagaattttctgcaataatagaaatgtcctagacctgccctgcccagtatggtagccactagtcacacgtggccacggagcacttgaaatgtgactagtgtgactgcggatctcaatttttatttcattttattttattttattttttcttttttgagacaggctctcactccaatgcccaggctggagtgctgtggcatgatcttggctcattgcaacctcaacctacaggtctaggtgattctcccacctcagcctcccaagtagctgcagctacaggcatgtgccgctacactcagctaattttttgggttttgtttgtttgtttgttttgtttttgtttttgtaaagacagggtttcgtcatgttgcccaggctgatctcaaactcctg

SEQ ID NO: 55 - DNA, 2001 bp, Homo sapiens
ttcattcatccatccattcattccattcaggaaacacacttcggacactgaatgtgccagaacctgtgcaggaagggggttgggggacaagctatatgggatgctgtctttgccttccaggaggaagctcacagcccaggcttgagggcactgaaatgcagaagagttctgggtccaactggggacaacgtcataaatttgcttcaggaactcccagttagctaaggagaaacattcctgcctcaggagagacccctctgcaatggtgaatacatagttcttgtcccaggaagacccccttctgaggagggagcctcagtgtgtcccaggatagtttccagtcgggtggggaggacccttggtctcgggggaattcccagtctgacccagcccttgccctaagagatctcagcctgatgggggatacacaaagcccactctctgggagctaacagtcagatgggagaggtaggctgaatagatccactctatacacagggggaggaacaggagaccgcatgtggaccgaaactaacgaaccactcactcacactaaactgagttagcaagtcctgttggtctttctgaaaagcaaagacattttgaacaggtgaaagcagggtgggttgaggtgttgcgcagaagtggaaaagggaaggaactttcttcccctgttccttccccgctccctccagaaaggtatttctggaactctccagttggtctgggatctgggcaactgctgtgtgtcccagtctgtcagcccctcagctgtcactctgctcccaaaaatagcaatggttgaggggagggagaggaagctgtttatctgggatagatgtggagaggtgcattagagctgtgcccagggagggagtctgctggggggttcctgtcccttctggatctttctgtcactctttggaatgttagtgcctaagccccccaccaggataacccagagtggggtgcttgggcggaggctggtcagcacctccacgtggtcatgactgaccagtctggggagggggagggccctgagtatcagtgacagactccaggggcacctgcccctgtggccaaatatggtccagagccactggctgaggcaagaaaatctcttgggtgggggctgggaggggcagactgaggctggctggctggaggttgacacaggagtgctcaggggagcagcatcacaagagggcagatcgaaagcatcgtccttgctgaaaaagtgagcatgggcctggggtgtgccctggactggggagggagacggcaaacttctaagacctgcaatgagagttcaggcctttggcacaaactaagcagcttcgcctgctcaggctgggagggacagagagtgaggcacagggactctcaacttgggccaagctctgagcaaatgctataatgtcctcatggggcaatagatttgggggttctgggggtgggtcccttccaggcagaatccccaccatcctctctggtgtgaggggaccctggagctccggacctcaggccaggagcccagccctgggtctgcagtgaggggctgaccagacagagcttctcctgtcccagcagatggcagaggtggaagcggtacagctgaaggaggaaggaaaccggcatttccagctccaggactacaaggccgccacaaatagctacagccaggccctgaagctgaccaaggacaaggccctgctggccacgctttatcggaaccgggcagcctgtggcctgaaaacggtctggggcagggcagggcacagggtgggagtgaggcagaggggctttggggctgagtggcagccccttcaccctgatagaaattgcccttcatcccagccgactctcctggatgcctggggttggagaagggtgcctccaaccccctgacccatgccccggaaagacagaaattcatccagagcagaatcagcccaatgccaggtgctttcatgtgttattcatggcatcttactaatggcc

SEQ ID NO: 56 - DNA, 2000 bp, Homo sapiens
agggagataatatctgagaagggacaagggcttttgagatgctgctgacattctgtcttggtctgagaagttattccataggtggtgttttctttgtgaatattcattaagctatacaattaggatttgtgcactttttctgaatatatatttatgcttcaatttaaagaaatgttaaagaaaaagagagtgcaaaatcagaggataggaggaagaaagagagatctctctggatttgagagcagcagcgactctggaagcccaccagggaggtggtaacagcctcgaaagaaaaagcgctggagcctagggggatctcaaaatgaggtttcctgtctcagcaataattcctaagccacactaacgaaccagaacactagttggcgaacactagcaaaaccaaagggagcacatgagctgggacactggggatctcaccaatgtggatggttgaccaatgacctgagtattcctccccaccccaacccagcctcctgggatcacactaactattgggaaagttctgaacttgaaacaatcacgagggagataggatttatgctgataatggctggagttatcctttccaacaacccagcagaatggggacttgcagtcataactcaagttgagttctaggaaataaaaaaagatctcttacatatctaaatttgaggatagagattcttaccagatacatggaactaacaagtctgggttttgtttattagttttttattttttaacagacttcctccttcttctctcatcctttcttctcagtttcttcccaatcgtcacacttgaaacagtagggcctaaagccacctatttggtatgcctgtaaggatggccctgtggatataacccaggacttactattccccccaaatttgctcttatgtactaaaataagcatatgtcaggagctatctcctccctgcaaagacacatgtacctttctgtacttcactggcacgtctgggatgtttgccagtgcgttcatttttctgtgcagagttgtccatgttagcaaaattctgtgacgtgttccatttgtttgatgaaaagttctactgcatgttctcctaagcacctgaaacagcaaaacacagcatttgtagagatcatttcacattggttaattattgcaaatgggttctgttctatctacagggtaaactgagaaatggtgtgggtgccggctgagatctaccaagtgcagtgacccaaagtcccaagcacattgtgctggtgctgcctgcagtaggaactgcgtaagaagtcactgttgatgttgcttacagaactactgaaggcttggattttatttttattttttattttttgatacggagttttgctcttgtcacccaggccggagtgtaatggtgtgatcttggctcactgcaaccgctgcccctgcctgggttcaagtgattctcctgccttagcctcctcagtagctgggattacaggcgcccaccatctgtaatttgtatttttagcagagatggggtttcactatgttggccaggctggtctcgaactcctgacctcaggtaatctgcctgcctctgccatccaaagtgctaggattacaggtgtgagccaccatgcctggcctatttttatttaaatttagctttactgagacataatgtatacacaaataaaattcatcagtgttagttgtacaattccaagttttggcaaatgtttacagccaagttaaataccaccacaattaagatattaatttatctatgatgctgagaagctccctgtgtccctttgcagtcagcaccctcccaaccactgctctgctgtcacaattgttgcctttctagaatgtcacagaaatgcaatcatagacactcttttttcacttagtgtaatgcttttgaaattcttccatattgttgcatagatcagtactttgttccagttgttctacatcctcaccaacatgtgtttgctcaatcttgttaattttaggc

SEQ ID NO: 57 - DNA, 2001 bp, Homo sapiens
cggggcctgcctcctgcatccccggcccaaaagcccgggccaagaaggacacaggcttcaatggctgtcatgtgttgcagacaacatggtgttgagatcttgcatggtggagggtgacgctggtccctgaagggagatggaggaggaggcagagctgggaacaaagggttaaagggcgccatgtaagagagctctccattcccaccacggagacatccagaccccagcagaggcccaaactgactcacaaacacacagccccatctttccccttccaaagaactaccttttcaagcaattccaggaagctggactcataggaggaatttgtcagaaaagactccttcagcttcagttgcaaagtcatacccggccctgcggatccagaagtacagcttaggacccagcagtcagggctgattcctccggagacacagaaccttcctgctcacgctccccggcacagttcctcttccccagcaatgcccctccagagcctcttggaagctcaggactggggtgtctctgtcactctcagggaccatgaagtcccaccccttttctctggcctcttcttgccacagggacccaggagtcctgccctctagccctcacctgttccatgtgagctgccaaggagggtcaagaggaggacaaggggcagcccagaccccatagtggccactgcgctcctgggatggaggagacactgaagtcccggtggcctctccttaacaggcctgtttctacaccccactcaagccttagcataagtgtttgaggggaaatgggaggaggaatctggtcaactggattttccagttctcccagtaagagaggacccaggagaggaccattcactgtgcttttggggaaaatagaaggaagtcccctcttttccactctgtgtcccacccctcccttgtgtctccaggtttgagggagtgagtgcgggtcctacagacagggaaatggagagggaagcggggaccagggaggcgtccctcctgcaggtgtctgggcccccacccatgcccgggcctcccaaggatctttagagattaattattaactgcagctaattttcatcattcttgacaccgaagggctcaggaatgtgggcccaaaagggaggggtggattaagccaagtttcttccagaacccaggtgtcctgctccctcaggttttttttttttaaacactagtcaagtgcagtagtgaaaagagtagaaggagtagaacaaggagttgggtctataatggactgtgaacactgcttccccagccttggtggctttctagatgagaaatctggtcatgggaacttccattgctgaacattctgttttacttgctctaacgcatcctggattgtcagggggagacacgagtttcctcagacatcctctcactcctgccttgtgcacccatgaaaaggggacacccacctactcccggccgagatgcctacaggcaagcgctgggacaggcaagcacaggacagatgtgcagagggagttactgaccctctttagaaactaggaaagtaaggcacagagagggtaagtgacctgctaaggtcacccagccaacaagtagcagagagattagaacccatccctctagccagacaaagagacacaccagccatggaaacttgcttgaacacacaagggcacaggccagcatcttcctatctgccacttccctatgcagcctctccaccttccccaaagccagcgggactgagcacagagagacagaggcagagagagcactgggcagaggtggggagagcaaaagcaagatggggaaacagagatagaggcctggtctgcactgtaagtgtaaagtgaggcaaccaatgactcccagacttgcacaggacaacaggaagaccacagtgggaagaaagcatggaggagaagagcagcaggcacaggagggacgagcaggaggtagaattgggaacacagagagatcttcctcggagaagagt

SEQ ID NO: 58 - DNA, 2001 bp, Homo sapiens
gtcaggagttcaagaacagcctggccaacagggtgaaaccccatctctactaaaaatacaaaatttagccaggtgtagtggcaggagcctgtaatcccagctactcgggagactgaggcagggagaactgcctgaacctggaaggtggaggttgcagtgagccgagattataccactgcactccagcctgggcgacaaagcgagactgtttcaaaaaaaaaaaatttcttatggctgaggagtctgtccttatcttggggaatcgagaagaaagctgtggggatatttttaaatgacggtttccccttgacaattgtccacaagattaataaaggaagattctgagcccttaccctgtgggaccgtgttctcataactgtccagcattgtgtctctgtagcaatccttttgagagccttctaaataattccaatctttggtcatattttcaaatgctgttgcttcctgaaacaacattggctgctgccacagggaccatatagtcttggggcctgggctaaagagcagcttcagcaggactggggaagagacaagagagggcaccaggaatgaaggcatgcagcataaaggactctgaaagcgagtcaggtgaataaatggaagaaataagaaataggtctcacatctaacaagatgagtacagcttcatggatgagggaaaggagttttgctttcctgggaggtctgttggtctggccagttttgacgctgccagagctgtagcccatttattaatctttccagaataaagcagagtccaagtgaacttgggcgcaggcttgatgactcctaacaggggtggcaggatctgtttccaatggaaatattatatctgtgtacataaagatatgtcaggagattatcctgcagaaaagagataccccaaatggagtctacctgccactgtgatcagataattccttagggagagaacttgatactcagttgtctaaactgggattccttagtcagaacagccgtaagacaggatggtgatgaggccaggggagttcacatgaggactgcaggaattccagcaagcttaggaaaacaggatcccgagatcccagctgtcagatgcagagcggaggggcagggcagaatcctgagaagcagagaaagatggaaacttttaaaggctctgagcaagagtctcagttaagtggggcctgcttccaggtccggaaggtccttcactaaaagggcttcaaactcccttcctcagcattgctagttaatcatgaatattcctgattctaagacattactgattgtaaatcactaacttaataacaactttcttttgagaggaaaaacagtgtgtattaaatgaatgtattgataagacacatccaaaatttaaaatgtgaaaaatatgcattttcattctttttctttttttgtatttttttatagagatggggttttgccatgttgcaaaaaacccaggccggtctcgaactcctgggctctagtgatccacctgcctcagcctcccaaagtgaggttttgccatgctggccaggctggtcctcgaacttctagcctcaagtgatctgctcgttttggcctcccaaagtgttggaattacaggtgtgagccagcacacccagccggtcctactagttctaacagacttgaaaccacaaggtattcaatggaatccagaaaaagcaccttccagattcctttctgaaaccagcgtctctcagaacctgagtgctgtgttggttgttatttgagtcccttcaggggaccgcaaaggaaggaaaagcctcatcttcattctataaaataatgaaggcaactgggcatggtggctcatgcctgtaatcctaacactttggtaggccaaggtgagaggattgcttgaggccaggagttcaagaccaacctagtcaacatagtgagaccctgtctctaaaaataaaaaaaagaaaaaaaagaatgaaggcaacaggctagaaacaatttataatc

SEQ ID NO: 59 - DNA, 2001 bp, Homo sapiens
tctgtgttagtaattaaggaaaattcacccagaaacacagattgtgtcaacacagatagcagacagttaaacaatgatctatggaccacagaccatgttattgagaaaaagagatctccgttgcttgggattaactacatggtgtgtaacatcaggaattcactggacatgtgtatctttcaatcaaaacatggtcacagcacatttattcattgattcagaattcagaggtcatagtcttgtcaaagagttatttcccaaaacctgttccatacaagttgcctctttacgggttagaatctggtaaagagacatcttaatgattgggtttgtttctctcttctccttccctgacatttccattccctaccccagccccaacatcctactagtggattccacagatggctctcacctctgcctctcgttgtttctcgcttttccactgttgctcccccagggtcacagggtgggctagaagtgtcttcagatcctcctgcttctctaaagtaacagcagcttcatactctccatctctgaaatcctcaggaaggaattcccctgcctccccattgccattcttctttccagtgacctatgagggaagaagatggcagcgtcagaagcagcagcctcgtcctgggtcattcttcacagacaatgccgcaggaggctcttggtccttctcccctgggaacagccacctttgagcagtgcactctcagatctcaagctgcgatgcacactgctacaattgtcccagaattaccttcagatgcttgtcctactctcttggttaaatcaaagcttgggttaggttgacagattaaatctacttttaatctgcttaatcacatttcttttaggaaaatcaaaaaccttctctgcttaattaaaactcaactttaggtacttttgatgagtttcgccttattgtaaagtttccgggcttacattctttcaataacacttcttccctaagacattttgataactacacgtgattccacactgtagtaataaaaccaaaggagaagatattaaataggaaatatgcagagaattcattactggtagctccattttttttttccaatttcacttgtttcatcacatcaacctttttccatatatgacattttcagagttcccttgcattacacctgaaagcaaagggcatttcaattttcaaataaaaaccaagatgaaccagttttcattttataaaattaatgcatcttacataccagttcctctactttcagtaccatcatgttggctgaaggagtcttgtatgtttttctgtcgtggaaggaatccctggagttggccctgctgggcccctccacaccggtcagcttatatagctggggccgggtgagataatcttccaacctgggaaccgaagtaacactcccacccccattaggagccaactcagaggcaggtgaattttcattccagactcagtgtctgggaagctgaagagggaggggaggacaagctaaccctgctgaatatgtgaatcaggaagaaatgtgagagggccattcctttggtggtgatcacatcgctcagcaatgcaagtcatcctatttgtcaagagtcaggggacagctgtctgttgacattgcaccacatcactgccctttttttctttgtcagctttcatatgactacctatcaagaaaatgtagatgccctacatatcacacccccagtaatatctttctgataagcagacttatcaacacttcacttaggggaaacttgtcccaggacatcctattccctgggtaaacagcctgaggggaagggatctgggccctaaaggcacttgtctatatctgtttggaattctttggagtgatgtctggggctaaataggccccatgacatgccaccttaccatcattataagaaattgaaagcattcagtaatgcacttttcttttatgatgtcactggccataattagcacatgacatgcttaccgtgttc

SEQ ID NO: 60 - DNA, 2001 bp, Homo sapiens
agaggtaattatccagttctatatcaaggcaaaaagacagcagtggatagaaagatcttagaagtcccactaggttcatccaagccaccatacacataggcagaaaaatcaaaataagatatgagcctggacagggtgagcaatctgggaaaagatgaacacagtatgctaggacccagaaatcatcaagtctatgaaaactaagccagaacacaaatgtgaattccataagatcaggaacataatctgtcttgttcatccaggcatggtaatctgccagaaatagtgcttaactgcaagaactgaatatttgttagataattaaaccatcaactaaatgagattcatgcaaccatgaaaaatgctgctataggtacacaatattgatatactagaaagttaaaaaatcaagttggaaattagactattccatttctgtttgtgtgtatgtatctacaaataggtggaaggatataccaaaatgtcaacagcagttacctctgggtggtgaggagtaatcttaaccttgttatttatccctatatgttcatttgtgaatgaatatttattacatcattataaaaaggatttttaaactatctgtatgtttaagagtatatgttgctactatgtaagagtatatgctgttactgtaaagacattgcattactactgttgacctcagagcacgagcctcttgcctaattctaggactcctaactaagtctttggagtttcagctggaagaatgctggaggaatacggaactcctcccatttctcacagccacctccaactcttaaaaacgcttccaactgcctcccagcacacaaccaagggagaaaactattctgtcaaagagacggtgccaaaaggcaaaaacaaaggtaaggatgatcgctggggaaagaagctgaaaaggaaaagctcagaactctagctggaaatttggctcacatccctagtatgttactgcatagtctggctttgttcaatgggtcgcttttaaatattaaagctagatgtaagcaaggtttgcaacaaagtccataagaaactcagcttttctcaaaggcaagaagagagcaggatttttgactggctctttattcaatagtgctgcttattaaattaccactgctacaatgtttaaagccaattacctgagcacatcataaggattctcttaccggttgtcccagttaagtaatgttgattgatcaactccttgacaggagctgatggcaaagaaggtagctgtgattggagctggggtcagtggcctaatttctctgaagtgctgtgtggatgagggacttgagcccacttgctttgagagaactgaagatattggaggagtgtggaggttcaaagtaagtgagattttcttgggtcttgaacaggttgtgttgttatttcagggtgaatcacagttactgatgggtcatattgagaaatttattaaacaactctgatcagattttatttctacttattgatgtggccataatggaactgaagtcataggctggcatctctcccccagtcaatactaacccaacccaggtagctgacccaggcatgtaaaagatctcttcttttggattcagcaattgtcttacagcccatacttctgtcattctttaatacgctaatattagagaacattttacaaaaatagaagtaacagggattcttctcaagatatcacttctgtttcaattattaaaccaaatgcttctttagagaccatgctcttatcattactatttttctctgacaaatgaagcatgtttgtttactgagctttatcaatgacattctagtataactgctgtgaaactctttgttaaatatgttttattaaatttattctattaatcaaaccaaaatattgataatgctatttgtctgtattagtccattctcatgctgctatgaagaaatactgagactgggtgatttataaaggaaagaggtttaattgactcccagttccac

SEQ ID NO: 61 - DNA, 2001 bp, Homo sapiens
aactggggtttctccatgttggtcaggctggtctcaaactcccaacctcaggtgatccgcccgccttgacctcccaaagtgctgggattacaggtgtgaaccaccgctccctgcgagaatttcttgaacccgggaggcggaggtttcagtgagccgagattgtgccattgcactccagcctgggtgacagagtgagactcagtctcaaaaaaaaaagaattttagctggtcatagtggcacatgcctgtaatcccagctatgtgggaggttgaggcaagaggatgacttgagccaggagttcaagactggcctaggcaacgtagcaggaccctatctccaaaaatttttttaaatgtagaaacattaggttatagcttgactgttacaaataaccatgtcttggtaataagggtctcacctctccttctggaattctcagtggcacactgggatttagcagcattgggggctggtcctggtcccacccagaacactgggcctcaatatcctcatctctacagtgagtgggttggcagagatcatctctaatgttacttccaaccctgtcgctataggtccattgcgaatattattatcatcactatttttcaggtggagaaactgatgaaccagttaaatatttgaataaaaacatctgttggcatggaaaagtgattatttcctaagctactgagtaaaacacagctgcactggcagttttaactctgttttcttgtcaatttttgttctttcaggaaaccttacagaaacatgaagccctcaaccatctgctactcagttattcggggctgacggcggcttctagaacatccaggtgttctgcagatgcgagaactcatcctgtagtcaccagatggagtcccaaacagccaagcagatgtaaggcctgtgctgtggctctgaggccctgaatacagaagggtcactttcttagtggccaaagagcagttgttgacattgatgtctaattattgaacacgaccagtcattttactgagctgcggtgaggaaacactgaccatagaagatcaagccaaatgagggattgcaaatttcctgattcttttgaattaggattccagatgggggcctcatttctacagcccccaacattcctatagccgttatcactgccatcaccactgccaccagcatcttcttgcagattccacccctgctccccagagacttcctgctttgaaagtgagcagaaaggaagctctcagaaaaatctctagtggtggctgccgtcgctccagacaatcggaatcctgccttcaccaccatgggctggctttttctaaaggttttgttggcgggagtgagtttctcaggatttctttatcctcttgtggatttttgcatcagtgggaaaacaagaggacagaagccaaactttgtgattattttggccgatgacatggggtggggtgacctgggagcaaactgggcagaaacaaaggacactgccaaccttgataagatggcttcggagggaatgaggtgagtcttgagatgccaggccagcctttctttggatgtcttactcccgttcttgagaggggaaggggccgtgcaaagcacttaaagagtcattgatggacccatgctgatttagttaatttattaattaatttggtttgaagccagcatagcatcgtgaagaaatccagccatctagcacccatccatctatctagtaacattcattatgtgttaggagctaggaatacagaggtgaacaagataaaacatttttgccctcaagttattcattgtctggtagagaagacaggttaaaaggaaagtaaggggctaggtacagtggctcatggctgtaatcccaatgctttgggagactcaggtgggaggttcacctgagcccatgattttgggaccagcttgggcaacctggcaagatcccacttctacaaaaaataaaagcaaattagctagcggtggtggcacgctcctgtggt

SEQ ID NO: 62 - DNA, 2001 bp, Homo sapiens
tccagacaagggggtgtggaggtcagagtccacaggagaggggcctgagaggaaataatggaaatgcggctagcaaacagccactaaatggacagaggagggggtgcccccagagaaagtaaatggggggtccagaccatgccagaatccatcctcagtccttcctaaagctggaatggttggaattccagaggaacccactgtggtcaagacagtcagccctcaggccccagcaccagtacacgggttggcagggatgatatcaagtccccgtgattccaaagcacctctgtttagtttgggagggaatgccagggctgagaaccttcctggagccagaggcaaggagtaaggatgcagattgttctgggatttaccaaggggctgatgtgaacgtccccaacacggggccaggctcttgttggtgttcagtaaaagtgggatggatctgacaactgataaggatacgttcccacccctcctgccctggcccagccctggcagctgatgggagagcagatcgtccaattcaagccttcctacggtctctttagaagacatgggctgaaacttggggtaatagaggttggctggcatatccatgcaggatgattgtcttcacatgtatctattaccttgtagaataaggtagaccctgattttggaacctgaagaccaaagtgcaagattagctctgctacttccatctgtggaccattgggcaggtatctctgggccttcacttactctttgtgaaatgaggacaggggcaatccctaccctaccaagtcattgggagtgaagacatgatgacacggtgattgtgaaaagattttgtcaatcgcaccagcattaagggtgcccatctccaggttcccccaggcctcaaggctcccaaggcctgagtgggcaggtagcacccaggtatagaccttccacgtgcagcacccaggacacagccagcatgaactgggcatttctgcagggcctgctgagtggcgtgaacaagtactccacagtgctgagccgcatctggctgtctgtggtgttcatctttcgtgtgctggtgtacgtggtggcagcggaggaggtgtgggacgatgagcagaaggactttgtctgcaacaccaagcagcccggctgccccaacgtctgctatgacgagttcttccccgtgtcccacgtgcgcctctgggccctacagctcatcctggtcacgtgcccctcactgctcgtggtcatgcacgtggcctaccgcgaggaacgcgagcgcaagcaccacctgaaacacgggcccaatgccccgtccctgtacgacaacctgagcaagaagcggggcggactgtggtggacgtacttgctgagcctcatcttcaaggccgccgtggatgctggcttcctctatatcttccaccgcctctacaaggattatgacatgccccgcgtggtggcctgctccgtggagccttgcccccacactgtggactgttacatctcccggcccacggagaagaaggtcttcacctacttcatggtgaccacagctgccatctgcatcctgctcaacctcagtgaagtcttctacctggtgggcaagaggtgcatggagatcttcggccccaggcaccggcggcctcggtgccgggaatgcctacccgatacgtgcccaccatatgtcctctcccagggagggcaccctgaggatgggaactctgtcctaatgaaggctgggtcggccccagtggatgcaggtgggtatccataacctgcgagatcagcagataagatcaacaggtcccccccacatgaggccacccaggaaaaaaggcaggggcagtggcatccttgccgtagcagggtggtgaggagggtggctgtgggggctcaggaagctcgcccaggggccaatgtgggaggttgggggtagtttggtccctgggtcctgagcctcaggggagggaggttgatagctactggggattttgtatatggcaacagtat

SEQ ID NO: 63 - DNA, 2001 bp, Homo sapiens
gacctcggtgtccacgattccccggcggcggcggcgctgcccaggtcagccccgaagctgcacactcaccccgtgggcgccgggcaccgtctgccgtctgctgcccgcgcggccgcgctccgctcgccttccgcccggggccgggctggggacgctggtccgcgccctcccgccgcgcgcgccgtctgcacaggccggggaggctcggcgggcgccctccccaacgtggagttatcctgggagcggctccagggcccagccggtgagggcgatggcagaatcccaatccgtccccaccccctctccgcgccctgggcgcgtcgcggccggaggctggcgggcgagagccaggcgctccttgagagcgccgcgcgttcgcaggtgcccggagcaggccctcgcggcgcccggcaccccctccacgcgcgcccctcttcccggcttcgcgctcgccctcctcactcgcttctccgccgctctcccaacctccccagcccactccggtcgcctcctcccagtctgcatgccacgccaaccccctccagtcccgctgccattttaactcctgagtggccagaggccccctctacctaacgcacacacacccgggaggaaggagttgggtggggccagacgctaacgcttgagctgtccttgtgcctagagacacaaggcttggctgagagaaggggcacagcccttgggtccgggaagagaagggttggcttctctcatactgcctcggaccctcttgcaaaggccaggagacgaatggggaattcagcagctttgtgaggacaggtgcagctcacagtcacagcatgcacctaagatcacatttttgctttgcatggtgctaccctcccgaagttaacagtggactaaggaatgtagtggggtgggtgagaatttgtcactgggcccaggcacctctcaccctaaagaatggagtggaagacccctttagttgtcatgcttttccagcattgcatcctgagccgtgcagctctagcattctggaagattccgtcctggctctcaatgaaggaggtcaatttcagtcaggaactggcaggcccagttaggggtttggcttcagggttccccactctgccagctggacatcatgaagactgggggaggaaagtaagaaggtgccaaaggtgggtgctaggctgctgcctggtggcttagaaaagctgggtgactgccactagaggagggcaaaggcccaatgcccaacacccatgccttggtggggacttcagcctggaccactcagaacagacatcacaaactcggctccatggcaggccacaccctactgatgggtctggaccagagcctgcagaaccaacaacaggggaatggatgaggtggaggccaaggtgtggagtcctgtgaggagtggcttccctgggctgcctgccgtcatccagtgcttgagcttctggccctgtcttttccaagggtacagccagtggtgactgccatgcctggtggcttttgtgaagttggcttgaaggtctgagttctgtatgcacagaggggattctcaccttcatatattgaatgagcacatattaagttcccactaaaccacccccatgttcccactctccacccctgctacggagcataatggcctcaaggagctcagagtgacaggttggaactatatgaaaccaattttaatatttcaagacgatggaagtgccatagaaatacaaaaagaaaccaccttaatggactagcattattcacaaaaggtttaacggaggagctgagtctccaactgagcctagttggagggataggatttgaactggaagagaatatggggaccagcaaaggcaggaaagtggagacaagcagagtagaatgggttgtctggagggtagggggcaagggaggaaaattatagagctactattagatgccagccttttctcatctctgtcatcttagactctgagttgactaatatccccttttaacaga

SEQ ID NO: 64 - DNA, 2001 bp, Homo sapiens
atggtgctggaaagggccactgtggccagctcagatgagcttcaaggatcccctggttaggagcatgaactctcatctgtgggcagagggatctcaggttaaattttaggaactgtagcaagctgtggcgaggagtgaggggactttttgggataaaattgtcttatgagctcaattgcatcccctaaaaattcattggtttaagccctaacccccaatatcccagaatgtgactgtaatggagacacggcctttaaagaggtggcttaggtaaaatgagatagttagagtggaccctaatccaatacagctagtgtccttatcagaagaggaaatttggacacagagagagatgccagggatgcatgccatgtaagaggcagcaagagcatggccatctgcaaaccaaagagagagggctcagaggaaaacaagccttccccaatggcttgacctcacatttctagtctccagaactgtgagcaaataaatttctttagtcaaagccacccagttgcagtgttttgttgtggtggcagagcaaacagataaagtagtaagtccctccagcaccctaggaggttctgcttgttgccattccccaaaggccatcctagccatgatgagggcaggtgcggttgtttgggaggagaccactgtggaacaaagagaaacaaggcttgcttgatgcagcagggagtagactacagattcaagaagggctctagaaaggtccagcaattcagcttgtcagggaaccccgagctgtatgtagttcttgctctcctgtgccctgcaggatggcttgacccatttctgttctcatgtcagaccctaagggcagggtcatctgggccacagacccaggcccggtgggcctggtgggctgacctgtgggctattcttatgccttcaggaaacccacctttaccacctggctgttgtgactggcatcgctgaggctgtttctttgtgtataaaatcgaataaggacacatcacgtgctagcaatccaccagcatagggcgaaggctcagcaaagagaagttctctcctcctctgctcttttaggtcagcagcaaatgcagatcggggtggggacaaggtaaacacataacttgggtggagatgtgattgattaataactcataaatcatctgaaaccatactttccttttgatagtcaaccctctgtaaacactcaatgtgttctcaccttgttatcattcccaatgatgtcgaatgcgtggttgccctctccagtataaaagtttgatgcagctttgcctggatgtacctgtctataaggagtcctgcttatcacaatggtaggtaactggttttatatataaaggctagaaaagcaaaagagaatatgtattcctattttaatatgcgtgtgcttctaccacatagcattgaatggtaaatgtgacctagttaggaaccttatgttattacctgcaattcaatgtgcagagtatgagagtctgagagcttgcgaaaattgtaatgagtttagcactagttctgttctctaagatctaagttgtttttccttccagtaattctggggttcaattagatttcataagtgtctctaattcttgggtcctatgtgagtaatttgaaattaaaaataatttcagagctacatgactgcatcgtttttgagatgagggaactgagcctggggaggggaagaagctgccactggggtctcaccgcaagcctgtgaatgagctgagataggaacccaggcattcctctctccctgccaggggctcccaaacttggctgtgcagtggaaactccgggggaattttgaagaacactgacacccatcttccaccccgacactctgatttaattgggctgcagtgagaacagagcatcaatttaaaaagctgcccaggtcattttaatactcaagtttgggaaccatttccctaggcccttttcactgtatcatgtgttaaggaaaaatcttgggtttggggc

SEQ ID NO: 65 - DNA, 2001 bp, Homo sapiens
aaaactgtttagtacaattctgacaatataatagtggcttaataaatgacagaaggagcaacctttgtttccagtttcatttgtccacatataccccaactgagatttgtttccgtgtcctgaccaaaaaatcacagattgcctctgtgacccagcctactgcaggttgtttctcccagcaggctcgaacccaagccaaggccttcaacacgcccaggcactgactgaggttgatacacacaataaggatcctaaacaagacacaatgtttccctcttcctgcctccctaccccttgaataggtggctctggtatatgaagtccatcccatgtaaagaggcaaggcgtacctttctgccaaagggaagaacactgttctttgaaaccaaaagccacttccagtctggtctgtggcctggacttacaaagaaaggcatcactcccccatgccaattcttgcatacacactggaaaaaaacaattgctctgttcttacctggagagtctgtgccttgagtcatggaggaatccagtcgggtcagattcactagcagagccaccacccaggggacccacccagaacccattctggagaaaggaaaaaaatgagatagtaaaatcgtcagcctcttcagaatgagctcataaaattcagtcagaaagtacccattaagagtataaatcgctgtttttctggcttccccaggattggaaactcctcagattgacaaccaatcaagatagaagagttttgcatcatcaggtactgggtaggatactttcacaaagttgtgtcatacaactcaaccattgtttgcctgcagaatcactgacagtaatttaggtatactaaaaatgggctgggagaagaagtaaaaatatatgtttgacatattatggggccctagaagaactaggcagactgtttattatgtattcttctattccctggcctgttctgaccaataggtctcccttcttattgggtgttgacattgccgacaggcagtgtgtaaattaagaaggaattaagagtatgtaaattaagcattctgaagcatatgcctcaagaagatttagtaaagggattatcagagaagaaatagaagacatagagaacatttggagagtacgatttcctataagccaaaatgaccacaaaaaaagaaaagaaaagaaaacatggaaagagaattctcaaagaataaaattttgcaaaaaggctcaccaggatacaatctaagagagcattattgtgttttatggttagtagggcaccaataaccttcaagaggacagcagagtgttaaaggtagaagccatatttcaaaagaatgaattggaaacaagaaaacaaaggccgcagattaaaacagtctttcaagaattttgaaagtgaaaacaaaggaaaaaatgggatacactttagaaaaaaacaacactaagaaaaaatacttttcttgggttttttaaattgacacgtaataattgtacatatttatggggtacagcatgatgttccaacacacctatacattgtgtattgatcaaatcagggtaattagtatgtgatatatatacatgatggaatactattccgccataaaaaagcatgaaatcctgtcattttcaacaacatgggtgaacctggagggctctatgttaagtgaaataagccaagaacagaaggactatattacgtgatttcactcatatgtggaatctaaaaaagttgacctcatcgaagtaaagagtacgttaggagttaccagaggctggggagtcaggggtagaaatgggtagaggttggtaacaagtacaaagttacagttagacagaaggaatgagttctggtgttttgggaaaagaaacttttaagacaggagagacttcaacaaggttgcaagtgaagtgcaaggtatctgtagaaaggagaaactgaagagaaaaaagagaagaaaggggacattttctccaaaatgctgtggtctgaatgtcact

SEQ ID NO: 66 - DNA, 2001 bp, Homo sapiens
aaatggcatctttggctgatacaaaaaaattagccaagtatggtggtgcgtgcccgtggccccagctattcaggaggctgagatgggaggatcacctgagcctgggaggtggaggttgcagtgagccgagattgtgccactgcactccagcctggacgacagagcgagactctgtctcaaaagaaaaaaaaagaaagacagcctgtccaatatggtgaaaccccatccctactaaaaaatacaaaaattagccaggtgtggtggtgtgcacctgtagccccagctacttaggaggctgaggcaggagaatcgaggttgcagtgagccgagattgcgccactgcactccagcctgggcgacaaagtgagacaaaaaacagaaagaaaaaaaaaaggaaaggaaaaggaaaaggcatccttttagatactaaccaggcctggggtcagatctggacttaaatcctggcgtgattgcagactggctggcttgactttgggcttgttctttctcctctctggggctcagtttcctcatctatgaaatggggagctatagtacctgcttctgaaggttggctgggcttttgtgtaaagcactcagccctgggcctggcacatagcaggggctcactagatgggccatattaactgtctgctccaaggctggagggtgttatagggaaaacagtcagaaaatactctccagatgcaaggtggccttaatatgtcaggatgtggaaggcagagagaagcctcggggtctgggatctcccataggggatgagtattttttcctggaatccctgccctgttgccacatcactaccctgaccatttgcccagcccagccctacatgcttcaggtgaaagcagagagagaggggcgggagagaaaagcccatcaagcagcaggcggagcctccctgcaggagtctggcctggctggaaggggagggggctgtggggccagggacgtcagatgccaacagcaccaggggaccgttaaagttgctgcgccgccaggtgggccagggtgtctgcagaggtccccggcctcctgggctgccgcctgccaccgcttgggctgcccgtcctgggtacgcaccgttggagcctcctgcctggtctctcgtgcttctctgtctcttctctaagtcgatttttcgctctttcttctgtttgtgcccctctctctgtctatttctgtcctcccccagccacattgttgcctctctctcattctctcttatctctttacctttctatttctcattcttttttctctgtaactctttttttctgtcccctttttggtctctcttttttctctgcctctttcattctctgccattgggttttctctgtgtctcattctcaattcctaaattgcagcaaagcagggacaaaaggaccttgcaatatgacttggggtaaggtgggccctgccctccgagggcattgactgagcccgctccagcaggattggtgaagaggatgtctagactggagagggctgctgtctgtctcctgggctggagggacagagcactggctttgaagtggtggcaccatctcaccccagggccatgtctggattagccactgtctgggcatttcttccccttgcccaacccactcagtcccctggggtggaaaggaaggggtgctgggcatctggaccagagctgcatctggaggtctttgccaagttcccaggaggcagcatcctctgggtcggcactgttggagaacgctcagggtctggtgctctcgtgcctgccccttccaggcagccggctctgattgcacaaggcagacctgtgactcctccatccagctatgcccctgctgcccagcaccgtgggcctggcaggcctgctcttctgggctggccaggcagtgaacgccttgataatgcctaatgctaccccagccccggcccagcccgagagcacggctatgcggctcctgagtggcctggaggtgcccaggtaccgccgga

SEQ ID NO: 67 - DNA, 2001 bp, Homo sapiens
tcatgagagaaaaaagaaaggacgtattatctcactgtgataaatctcataatattggcatactccctaagttgggcaactttgatcagttttctggaaccatcataaaaaggcctttaattggataaagtctcgaacttttcttctgtggatgtgaatatcttgagaagtactggaatgaaacaatttggctctggtttcagtcatcttcacatgtttcttcaggtgctttcttaagctattagggcaaaataagctggtagaaaaaaggtgttacttgtaggatgaaagaagcaactaaagagaagaaactttctctagaaaaatgttacatttacacatttataattgttaacaacttttttcttcttttctttttgggagactaaataatgggatgatacaggaagaaatatgatttagagaaaactgattgagccaaatttttcaaacggtaatatgggtaatcataggctatggaaagttaaactgagtctccaatgtttatgattatgatgtttggctaggatataaaataaggcttcagaatataaaatatcctgcagattgagaagttttaaaagtagttacatacaagttttatttttccaacgttcctgccttttctaatgtctaaggatgaagcagtgtacagagaggaaagaatgttatatgtgtggtctgcttgcaagtgctggatgcctcctctgcctgctccaactaaactgtctgcttatgagaaagtcattgctcctctgagttcctgcctccctatatattcaacattagaattttgccaggttatcttttagggtcgttcaactctaacatatatatgagtctgtgacactgaactcagacacttcagaacaggaaccctggatttctctggatgtggaaatctttaaatctatacctgactggtaggacaaccaatctgtcttcctttcctctcctctcccacttatgaagggagtccagtcagaaacatgtttgtctacgaggtagtatatctggtatttctgaggaattcggttattatataggattatagaatattcaactttgagtagggtctgtggactccatcaattgcgttctgggggaggtaccatgaaagaacattcctactttagaaatagatattctctgttcaaacctgacaccctgacaggatcctaaaggatggaagctaaagtggtgtggtatataagtcccatgaccttgaacaactggttttatgtgggtgtcaccagtgctttataaaagtaaactgcatcgccccgagttcagtcatactgcaccagctgagcaatgcatggagtggacctgtaggcgacttgcatcgtcttcaacatgaagatagccacagtgtcagtgcttctgcccttggctctttgcctcatacaaggtgagcaatttgtgtgtaatctaagcctcttgccacacatctcaaagccctggggaagggacttttctccccctactcctgccaaaaaatgtttacgtatgcatgtataatcctgaaatgtcttgccagacacagagttctgaagatttatatatgtggctttataattatgttgttatctttagaaatggcaagtagtttgagataatctgaattttctaaccatgagaaattcttccattaattctttattaattcagaaaactgaatagattcttcttcatcttcctcttgctaaatgtatcaattcataatgcatattcttatatatgtaatagacatttaaaacttaattgtaaataatatatgtattttaatgttataggtataaatgaaatattttttaaaattcaatcatctgttcttattttaaagcagtggtatcttctagcttattttagtcttgtgttttcaaatgctaaatgggttgaaagcatttctcccaaagagtgagggacttttgtgggagctggaaggtagatttttatatttgctatttaaccctaagagaacacaaattgcctagactt

SEQ ID NO: 68 - DNA, 2001 bp, Homo sapiens
gttatagcagccctagaaaactaagatggaactcgccggctggtcactgtcttccctcttatcacactaccttcccttactgcagctacttatttaactgtttcccaccaaaagaccctaacatccaggagtgtggggcccacatctgcctaattcgccactacggcccaagcccacctctggcacactggaggtgttcaagatgtgagctgacatatgaaagagctgtcctgactagttctagaatctcaggcatgtgactccccctcccagaatgtcaatttccccatctctctgcagagggaaaagaaaataattggagtagatgacctctgtgagtccttcgggctcaaaagatggccgggactcaccactgaaggtgtctgcatccatcatggtcagatcaggccatgagaaaccccactagaggtgctaaacagagggaatgggtcacagaggtgtgggaagcctgagaaaccactgcggccttcctgctggacttcagagaagggctggcctcattttatgggctttctgaaacccatattcagagaaacacacccaattcagcatggactggcaatgcctatccccaaggaaactccaaaaggggtgagtctcctgggcagtggtcactgagttactacaaggcctttggggttggggtggggatggtgggtcaacaggctcccctagaagccctgtgccctgacaaagtaaacaatggcaaaacatgtgcttcccagcactcacctccccacctccttctaacatcagaactgggtttctgatgatgggagaaaatgtaaatgaaatcaacggaaggatgatcctgtgagtaatctgacaagggagaaccagcttcctctcccatgaaccccagctgccttggctttcacccagacccctggcccacccggtgtcctccccagacagtggtactcacccacactgctgagggagctgctgctttctgagtctgagggcatggtggacaggacgctgtaggtagaacctggggaaagaggggagaccatgttactaaccccaaggggtcactcgtcttcaccccaagccagctgatcatcagtcaatgccaggccatgagagctacatgcaccaggcactagaaatccacatccacgggccaagcagaggaggcgcgctcagcagatctggacttcggtgtttctgccaggcctcccacctcactttatcggcccctcctgctctgggcaggcgggcagaggggaacttggagtcagtcagctggcttcttgttgggacatctggggaaaggcttagcaagggagtgacatgccccaggtaaacatggggtgtgggggtttcctccacatctctagcaatatgattctggcttccatgcagagggtaacagagcagaaattaaagaacaactgaactatctgttctttctctgactgttctgctctttaggttctaactgtattcaagctcagttcttaataatttacttagtatctgcccccttattaaaagttactcctaaaatgatgagcaattgtaccatcaccaaagccagagggtagcacttttttttgtccacacttaatgttaattcctggttgtggacacacaaccactgtggtttggtttgaattcaaatatcttcaaaaccagatgttaccctcccagcaccgggaagcccagtttctgaacctagctctatgtaggtgcccatagctgggtcgtgctgagcaagtagcttttgttttctggaactctgtttcctggtctgcaaaatgaaggagttcaaccagatggcttataaggtccctaccccatcccccatcccccatcccccatcccatttcagcaatttccatgattaatggaaacagaaggtacactttgctcatcaagactatggatgacataaaattaaaagggagggcaaactgactcagagactaaattgggagtggacaaagcataacacacagagatggcaggtcagat

SEQ ID NO: 69 - DNA, 2001 bp, Homo sapiens
aaatgattgtgaaacattttttgcaactgtaagagggttgatacttttcctgaatattattcattcaacattcatttatttctttgacaaatatttaagagatgctcatgatgtcctaggcattgagctaagaactcaatgttgcaatgtggataagatccagtccctgtgtgttcatacttatttaatactattatagacaaggcctttaatacataccatagtcatatatcaatttttaattagcaatatgaaataagagaacaatgggactttccactcaatttgacaggtggctaaattttcagttactgaatttactattttagggtaatctagcacatgaccaaaatttaaatgcacagaacagaagatcagagtaagaagagcttccatttggattatcactttttctacttggagtcacatactgtactttaaattgattaagaacatgttaaaggtccaggtggtgatttccagctacaagtccaaagtatagaagaatgtgtttaaatcctacgtttatcagatccataaatatagttccttctatgttaagtggctttatttctaagaacccagccctcggtcctgggggtgcttcccagcccgctgctggaattcctgatcgtggcagtctgctgaaacatgagctcagttaccaaatatagctgagtggagtaagaggacagatgagagtctgaggccacagttgcctcaaagtgaccacggaccaggcagtctctaatgaataggcaaggccacaacctccattctcccagaaaagaagaaatgctcatctgaaattcatcacctctctggagtcttcaaactgaccaagcattgaaaaggtctgtaaggtgtatcttaatatactaactagtagggcaacattttaaggacttaaaaaaaaaaaaactaaaaatacaaaagtttttctgaagatatgatactatttacaagataccttaattattcttttgcttggagaggaaacgtaatgagaatgttacggttttgccaaaaacagataatacgtaagatttgcctgcccacttcatctctgcctttgctgtcagagaattacactttctgttataagcttaagcatgctaagatggtgctatcaattccaaaaggattaaatgtatttatggagccagcttttgtcaagatcgtgtatgtgttatgtttcacagaaaaggaaaacgcagctttctagaccctcacccatatttaagctagataagtgatgatggcttaaagtgatagaccttagatttcagtaacattataaattgttaattacactttcaaaagattctaaaagtaactcactggacataattaaatgagttgggatagtaacagcaggtattttaaattatattcatgcataaaatttacatacatatttttaataagataacgcttactaagggaaaccacagtgcaatctcgagtccagtcaagtttaaacatcacagaataatttttatcatgttgttttaagttgcacatgaaattgtcttgaatccaatataggaggttgacataaaatgatttcaggctcatattacttattaacatttgctaaaaatactaattgccttctagtatgaataatgtggtgacatataaagccatttcttggcaatataaaactattacaaatctattcaatcacatatatgcagaaattaaatcctcatttcatgataaatgtgccttccaatgttttttctcacagaaagaacttgaatactcacctcaattattcagatttttttttcagatttttaaaatcaaagtctctttgacaagttattgctaacagcagaaaaggccgatgaggtttatctggccactaactgccctgtaaatacatgattacacattcctagaatataaattagtcctctggcatcctagaatctggtatggattagggtctgtatcccaggggttctgtgtcagctcttcctctgggtttctctg

SEQ ID NO: 70 - DNA, 2001 bp, Homo sapiens
cctctttttggggaaaaggacttcaagctcaggttttctttggtggggttgggtattgcttcttccatattccaagtctcaggaccctcaggggcaggtgccaagtgagagtgacccccagaggtagaaaggacccagcatcatttctgaggacacctggtagaggaggagcctggacacttggcctcccatcctggctcagggtatggccttgacacatcttcatccttgtttcctcctttggacaatggagatgttcagactaggtggtctctgagggccctgccagctttagccatccctccccacaccagctaaagggactaattcctccgaactttcatgggtcagatagaggcagggaagggacagccccagccctgggaggcaggtctccttaccttcttccagttccttcacaggaatcacacacttgaagccacagtgcaggtaacaacacttcctttcccccagacagtcctggtctgtgtgacactggggaggatcggacttgaagcagcgtacgttgtcagctgggcaaacccctgctttctctatacctagtggaggaagccacaaggtgatatgagaactccagccccagagggcccctcaggacctctccccagcccagccccacccagctccaccatgtctgctcaccctctttaactccttccacagccaccagggtcacaagaacgagagacaccatgaggaccaagaagctgctggaccccatgttgccaggcaggtgcttggctgggagctgggctgagcctggctttatagtgttcctgctgggcatgccagtgggtggagctgaaggctcaaaatggcaagggaggctgcacttcccagctctatttggaaatggccccttctgaggagggacttggggctttccctaaggctctgcactggaatagatgaagaaatccaagagcgaccctcgttcttcacacgaggagaagaaatggacacgtgattgaccattaggcgccaccagggccaaactatcttatggaaggaggaaaagaagcacagaaagggcatgaaattactcaagttcacacacgaggcagtggcagggccagacttcctcaactccaccgtgttctctatcagtccctcttcatgcctgattcacctgggaccaaaatcttccctccttcctgtgctgccccggacttgttctcagttctcacctcaccccaaggaagaatctgaatggcagtgcttaaaatgtctgtcctgaacattcactctcagccttgcgctccagcaccgtgtgctcctgccccccgccaatctcatgtggcctggaggaaagcagattttggacctgtcaccagctgtgtgactttgaaagcagcttcacctttctaagcctccgtttcctcattcatacaatgagcataataattatactgtatcatcttcataggatttgttttccctcctctgcccctgcatcccccaaattcctggatttggaagaagacatggacgtgtgtgcgtgtgtgtgtgtgtgtgtgtgtgtgtgtgtgtgtgaccaggcttttggggtcctttctttgaatgagtactaaagggaggaaaatccttcctgttggaaaagttgcagatataagcccagaaactccagaaatttggtcctggccctggtgagagagaaattctgagaagataaatctaagatgctgataaaaagagatgagagatggacagggagtaatttagaggatatctagtccttcactaaactttttcctgagatcagccccactcatacccagggttatgttaccacccaatacattcacctttctgcctaagctggtctgaattggatttctgtcatttactaacaggacccctgagtaatgcaaagatttgggtttggggtgttccacatcctgaagtgtggaagggactgagttggggttaagtggaggcagggaggatacctccattcctggaagtgagcctactccttat

SEQ ID NO: 71 - DNA, 2001 bp, Homo sapiens; identical to SEQ ID NO: 70

SEQ ID NO: 72 - DNA, 2001 bp, Homo sapiens
aaaaaatacctaaaactcatcaccccttaattatgtgagtacatttttctattatttgtgctgtcgaggttacttacgcccattatgtctgcatgatgggactactcccgtacagtggtgagcgaccttcccatgtcccggttcaaagacactgtagtggtggtttgaaagcagccctgatgggaccactgacatcacagaaattggcaaatgctgcaaagtagggctatcccaccccacccagagatctaactgttacaacatttacaagcctgaaagccaatttgaggcaaaaaggctgtcatggaagaagattctcactgtggagtttgcgcactcagatttgagtccaggctccgtctccacccactgtggggcctgggctatttccccttgagcctcgcctttcctatctgcaaaataagactaaaagtacagcagcttttgcaaattgagcatttgccgagtgtggtggcttgcccctgcaatcccagcaactcaggaggctgaggcaggaggatcacttgaggccaggagttcaagaccagcctgagcaacatagcaagacgctgtctctacaacaaataaatacataaataagcaagtcaggcaggggggagcacacctgtagttctggctacttgagagtctgaggcaagaggattgcttgaacttaggagttggaggctggactgagctatgatggtacctctgtactccaacccaggtgagagcataagatcctgtctctcttaaaaaaaaaaaagagcatttactacctgtcaggtgctgaatccatgctcttgctaaatctgccagcagccctgtgaggtgggtgcagtcacgtcccttgcgaagatggggatttgggggcgtgaagaggataagtacctcatcaaaggtgacagatcagatttgaaccacactcttaactgctgcggtttattggccctacaataatagtgcccagttcacagggtggtttgggggattacacgacgtaatgtgtgttaaagggcttagcacagattcagctccagaggaacatagtaaatgttgatttaccgagaggcagaagagcgggatccacatccacccacgtttgaatctcaacttggctattttctgattgcgagatctctggagtcaagtcattttgcttatctaagcctcatgttcccccagtgaaaatggggataatcatagctactgaagagctgctcagaggatggagatagaggatgtaaagtatctgccgagaagtgggtactttatgggagtgagtgttcgggattatcttttattttatgtcacgcagacagcaactcttcccagccctgtggaggaggccggaggttggggccggagccctggaagcagggccttgctcgggcagcttctgaccctcccctcctggccaggtcagcccctcagtgggccctggccctagaaggtctcagaagcctggggacgctgttgctaggcaccagttgctaggctacacattgtttctcctgcttggcctcctccaggagggctggcttctctgcagagctctgcttcctgcagcctctccccgacctcagagcctctgcagaggaaaggtgagggtggggccgctgtggttgggcctcggggagagtgcagccaattcccaggggaggcaggcaaggccagaggcgggcaaggccatggagatgagggagagagaggagccgggggctgggagtgagcgcctcggcctgacttcttgggtgggggccaagaattccccaggtagtgggttgtcggctcattcattccgagaggtgggtatgtaaggttcaggtcaggggccgagtagtcaaacacctaggtctgaatcccagcactaccacctattatggagcaaatcctttatcacttggaacctcactgtcccagtggtaaatggggacaggaagagttaataagaaggtgtgggaaagtgagaggagacatgaggactcttgggataagtgt

SEQ ID NO: 73 - DNA, 2001 bp, Homo sapiens
caaagttgcaaggcactactatagatctcccatttcttccttttgttcaccagaacgttaacaagagatacaacacctagaaaacagagagcagagacttttctggatcaaacagtaccttttttttctgtttctgtctccttgtgactcaagggagaaatgatcctttctttctaagtgatcttatttctctctgtcaagaatccgtggacatgttatctggttagagctcagcctgcagccaccacgcaggtaactcactcacgttagactataactcacctaactcgtttgtgtcccagccaaatgtgtaccagagtaaaagttcagacctgaccatgggtaagtttaatgattaacataagccactcaggctttttcctcagggcttgttagtaatgaccaaaggaaaagtgtcttacatttttctccacttactgcatgtgccccagataagaattttaagtaaaagtccacccagtgagacaaacaggcattcaacattgaaagaatttgccagcaaatcagcgattgaacagagggctcaagagaaaggaccactagagggcagatccccattcatgaacagtctctcactcttcatcccgaagagtgactcgtaaataccaggttatgggctgttattcatatacccactgtaggggtatgggtctaaagaagtacagaagggaagtaacttggtccaagtcttgctgagaaagggttgattgattctcttcatttttctactcctcatcctccctcttgaccttgcttttttaaccaagaggaggtccttgacttgtactcatcaccctccagtcgtacccagaccatgggcctcagtcatcggttgaactggtgggcctaggaaggtcaacaacaccttcttacttggtccatgtctgacttctctgttcctgtcccactgcccattctgattcacatcccccaattcctgatcatgtttgtctacgtctggcgatacctgaccagaagacgtccttatcttctatcctctccctttcctttgagggagaagtcattttggtaaaatttgatagccctgagtctccaccttcaaggaccagcagtagacctggagaagcatctccagcaaaaccagattaaagccatggaaatacgaccacaactctgatgacagtagtaaaactatcctctagaaagtgtggaagtttacatcaactctgctgtatatcacagtttggcaacccgtgacaagctgaaaaatttttgtagccctgaagctagtggtgtagagagagggtgcttggccagattggggcaggtagggggtcagtcaccccaccaccctgcctgcctgctgaacctctctcagtttccactctggatctcttccatgcaggtcaacaagcatcatttgaaccacatttgtgtgcctggcatgtgggtaatgggaaggagggggaagactagtttgggggtaaaggagatttcaacattattagtaacttttatttttataaaaatgatatgaatcacttagataattcaaagttatatttttaaagagtatgacacagtttctgccttcaaactgcttaccatctggcagaggagacagaaatataatcagattattttattatattgtagagtctcctgataagtttcctaagacttatgttttatttcttgttatttcaacaagaggtttccataaattagccccacatctatccttggctaaaaggcctataataaatatttataagtatttttggcagcctagctatcaaggccgcatcctaaaatcaccccaattttcttatgaggagacagccacctctctactatggactaaaagaatcctaatgacacccaggactttggtttctgtgctctgtgttataggtatttcgtggtggacaatcaagctctattgtaaaagaaataaaggacaatgaatcatttcttgccttgaggcctaaaaggaaatgttgtagaactctagagaaa

SEQ ID NO: 74 - DNA, 2001 bp, Homo sapiens
ccaagaatgagttttcaaaacccgagttcagaagaaggattctgagtggttggttctcagatctctgaggaagcggaaggatgaccctagggggagggccaccgcatgttagaacccagcagggggctgtgacagacaggctggtgctcggagcttcatctctttctgccgttttcctctcagtttttgctctggttatgtcatatttctcattttcaacaccaacatgagcctttgtacagacattcctccacctggaaccccatttcagctcatctacctgttgctttgtatttattcttcaaaactctgccgtcttccctgaccttccggagccggtgtttgtggcgcctcctgctggtcactgggttcaggcagtttggagctgggtggaaggaacgggtagtatattaagtcttgggccctggaccttgttggctgtcctctccctgctgacctaaatgacaaagggaggggtgtccagtcagagtgtgagggctgtacccgtgggcagctgagggagtgcgagccacaggccaggcactctccttctcccattagctcagccgtggcatcggacttgcagcttcattttgggctgccttagccatgaagctccttttgctgactttgactgtgctgctgctcttatcccagctgactccaggtaacctgaacctccttaaggaaggggcagggcttagagactaaggcctggtcagagaatcccaagggctagaaggatgggctgcaggttgtcatctagcaccacgtataggaggcaggaggcgcctcaccagcagcaggtgccagctgatagatgacaactacaacatccagagctgctctctcagcggctccatccctcctcttcctgcccagctccagggagcatgtgggggtggcctagtgtgtgaggaggagtgggaggggtgagcaagagaagcaggaaagaggagaggcaggtatggcccttttgcaaaatagtccccagaatgaacgaagctttgtgaagatcacagctaccttaatgggagagaaagctcatttgtacttagctgtctctgtgccagactctgcgctggaagctaggaccacggagataaatgacatggtgccaggggaactaactggaggagacagttaaatagcatttataacccagggtgacacggctgtggcagtgggaaaccagggggctgggtacacatggtccagcccaggctgagggaaaggagagtcctcctggagggatatcctgagtcctaaaaagtgagtcgaaaatagaaaaggaaggagaggggaaagcagaagggtgtcccaggcagagaggtcagcctatgcaaaggttggatgcgggagagagcatgggagtgtcggggacctgcataacatggaggggtgggttaggagagaggcgggggtaaagctgaggaagcattcaagctccaggtctcagtgggcctcaaatgccaggtgagggacccatgggaaggtttaaacagagaaagaccctggtcagacctaccacccaccaattccagcatgagccacctatcccttcctgctcatctggcagaagtttccacccaccctaaggctgtcggctaacctttctacagagtcagaaacgtgtgaatttcctaaacaagctgacctccgtccttgtgtcttcctgaggccctaaaacaccactcacctggagtagggaggaaagcccagaccccgggttaaaacctgccccatcattttctgactctgtgagccccaacaagaagctcagctgctcagagtatcaatttcctcatttctcaaactgcaataacatcaagtgtgtaataatatcaggttgagggaacaacattgaacacactgaaattttggtctgcaaaagaaatatggcttctctattttctctaactcaccaaactttcccaggaacttccatcctgactgtgggagttagtggtgggaggagcaggactacagagataaatgaca

SEQ ID NO: 75 - DNA, 1920 bp, Homo sapiens
cccttggtaggtcctatcttatgctatccttggtctctcatgctattagaaattccacatttttttttcccgatcctttctggtgggcatctctctagtgtgactcctctcttgaggttttggctctggagggctctttccaaccccagaacatgttcctagtcagagccttttctcccagtctctcacatcactgccacatggtggttgcctggattcagtggagaaggtggctgcaaactctagttggacactgactacacagatggtttcaaccaaacccagactgtgcactgcctgcctccccataaagcctgtgaggatggggaccatgactcttgctcacccctggatcctgagccccaggaacagtggcttggacaaaatactctagaccctgctcagtggccaaatcactgtgtggtatagaaagagttctgtgccatccacgagacccacttccagacagttttccagcaccacacttattagccctgccttgatggagacagcatctccttggcatttaagcagttatttggcttcaaatgccctgagcgcccacacagtggtcctttgctcactaggtgatattatcactcagtgtggaaggctctgcccctcgttctgcttttgtgttttcagttaatctcccccatcagtctggtagctcccggtcccatttttgtctttctgtctgtgtcctagaagtatgactacctttccctttggcctgggaacttctgtgggcaagttcttgcctttgacttccctgtcagtgcccagcctgagttctgaacatgagtaggggttcaggaagggtgtgatccaggacgacacccaccttgtcgatgaaggccgcgaatctgctgttgagggacttgatctgctccttctcctcctgcttcacgcactgcgcgttggggtctatctccaggttgaggggcgtgaggaggctctcgttgaccgacacggtggtgatgcatggggggctgggtccgcacacgcccccggagcggtagccgaagctgcgtccgcaggagccggcgcggaagcccccgcacacgctgtggctgccaaagcccccggtgaggccgcggtagcaggagatgccgcggtagggggcggcggtgatgcagcagcggcttggccggggcccgcaggcagagacacagctgaagtttccagggcggaacccacagcctatggagttgaagccacaggtcatgacggaggttaggaggtgtctgaacagtggagtagatggcagaggatggaaccaagggcctgtgctccctggctgatggcaggcccttttatgtgccaggggcagctggtgttaaaagagggagcaaagagacaatagctgaattttattggcttggcatcaccctggcctcttaagctagaaccctgcttgcctcttcagtgtggctgcatcaacaatccctggccacgggccctgtttcatcttcaaagccctttataagcttcctccttgctcctgcccctcccactggcactgctgtggagcggggagaggggttcctgaaagcattagttgcattctgcggtccaagcgaggggaatgggtgcagtgacctggggtaaagcctgctctctgaaccctgttctgaaaggggcagagctaggctatgggccctggacccatggccaagccctggggtggagtggctggcctgtcccaaggagggggctgcctctgagacaacctcttgctgtaggctttcctgtctttttctcaaaggcgggttcctttcctcattgtcaagattggccgctcctatcctattcctgctcagagcctggtcaaggatgggagtgggggtagagatttggggtgggaggagggcatgttctttgcactttgggtattcctagtgcagggacaccatctgg

SEQ ID NO: 76 - DNA, 2001 bp, Homo sapiens
cattattattcccattttacagaggaggaaactgaggtaacctgatcatgaccacactgccccacagtagaggagccagaatccaaacccaggcagcctgccttgccaccagagccacgcttttcaccctcaagctgcagttaagaagtatcacgccgtggctaggcccacggcctctggcttcacatcccactagctctgccatctttagctgtgtgaccttgggcaagttacttgtctgtgcccgagggtcctctgtaaagtggggatagtagtagctgcttcactgggttcccttcaatgagttaatagctgtaacatgtttagaatggtgccctgcacatagtcaccagccagtacatatgggccattattcctgaaccctggataggcacatgtccttgaagagtgtagaattcttcaggattctgaaatgccagcctgtgggatcctggaatcctccaatagcattctaacagctctcagctgcagaactgtaccctgttttctagagctggaaggtgtgaggctgcctgtccacccagctcaaccccacagtaggcaaatgaggtgatgtcaaagccttggctggggcccagccccggtaacctcccactctccctcccctgtgtgctccctcgctcagattcacacacacactccaggccagccgggcccagagggaaggtgagagggcaaggagtaaaggtggctgggtgtgggtccgttgaagcgagccgcctccagccctgttgaactggtgggcccagggactggagcgggattgaaagggatcttgctctcccttgaagccttgagttgcagcgatttcagtgtcttctctccctgtgtaagcctgtctcggtgtttaggctgaactacagccaccccctctcccgggggtgtgcaggccagggactggccaggcagccatggctgacgagaagaccttccggatcggcttcattgtgctggggcttttcctgctggccctcggtacgttcctcatgagccatgatcggccccaggtctacggcaccttctatgccatgggcagcgtcatggtgatcgggggcatcatctggagcatgtgccagtgctaccccaaggtaggtggtagtggggctgggtggggccaggtcagctggggccaggagggctggacactgtgcctgtatgccatgcctcaccccctatcctttagttgtcttttcattcttctcattttgactcggtcttctctctgctctcctgagcgtctgtcccatccatccatccatccatccatccatccatccatccatcccttagcctaagggttgatcagaaggcccctggatctggagccctgagccggaagctatggtatagacacagcaaacacaacacaccatgtgcttgaaacaaaagctaatgagcgctgaccccctgccaggcactcctgcgttcagggtgcagttcagccctcatcctaagccttttggtgctttctgtagttccaccttctgagcctttctgaggtccagaaagagaggctgcacagctaacgagcaaggtttggagctcagatgcgtgtgacttacacgtcacccttcaccttgggtcttctacctctgggtcccagagcgactgagagctccagggtaggtcaggtctaaccttggtggcatgcactgggcagtatcctttagagtcttcatgtattctcagccttatgtgtaatttagtgatgggaagagcactggtttgggagtcctgagttctagttcaagtcctgttccaaactctctatgacctttgtctttctgagcctcatttttttcatctgcaaatgagatccctgcccctattttccacagagcttcacagggtcattataaagatcagagaagagataagatataggaatattttgtaaactgtaaggtgttatttatagatgaagggctaccactgttacccatactgatcctcccaacagctctgtggaatggaaatgcc

SEQ ID NO: 77 - DNA, 2001 bp, Homo sapiens
gtagcagaggagctttcaagcatgaaaggagcctatgggagggagcaaaggcaccaaaagatccctccctccccccaggcacccacaggaagaaccagcaggccatgaggtcccacaccacccattctgtagtgtggttccttgagagatcagggccacagaagacttagaagcgcctgagcagcttgccaggaagcagctagggacaggtctgacccctacatcctgatgctatttcttctggttagtggaaatcccttcaccaacaccctcccagtctctaagaccctttcttcagactctccccttctctgccactgcagctggaggtaagatgtggggaagaaaggctagtcttttcttaaataaatcccagctttggctcagtccccctcctgacagttgttggaaccctcaggtggagaccaccaaggactctggggcaccagggactagaggacaaggtgacagaaggcataaggtacaggctacaggcagctctggaaatgccatggtgggaggggagtcccagtgccatcctgccctccccttcccccgctccacactcaccatgcagacaaatatggctgagctggtccacaagttccaacaggttcatgaccttcaagcccttgaggacaactttcacagcctccttttctccatactttgaaatcagtaattctgccaggtccaccggaatcaggccctccaactcccctctggccagtgggggctggccctcagacagggtcatatcccgtaagtagaactttaacttcttgaaatcgttctcctcaaggtcactcaaggcccagagcaatgcctcccggggctttctggccttggccatggccatggtgatctgggggaaggatcaagtccagaccagaagaccagtgacctggagaaagaggcaaaaggagaggtccactgctgagaggcccaccctgctttctgggggcttggccaagttatagagcggaggccagggagagacgagatacatgtgcaaagctccaagaaagacaagcagatgaaacgcaggggaattttcctagtcattcccctctgcctcttagtcaggtgacacgtgactgtgttggcaaatgggtaggaaggtatgtaggtgtgttaatctctcaatctctctctctctcccccaaactctatctctgtctctctctctccccccactcccttcctctttccctctcaacacgttctttattaggctctgtatgcctccttagacggatggattaagaaaaaaaagtctaagttttttattttattttttttggagacagagtctacctctgttgcccaggctggggagcagtggcatgatctcggctcacctcaacctccacatcccacgttcaagcaattctcctgcctcagcctcccaagtagctggaattgcaggtgcccaccaccatgcccagctcacttttgtatttttagtagagatggggtttcaccatacgggcccggctagactcgatctcctgacctcaggtgatctgcccgccttggcttcccaaagtgctgggattacaggcatgagctactgtgcccggccaagttttaagttttaaaagtgagactgtccctggctgtgtgaacctatagaaagctggagcacatttgctggaactgctgtggtatggcaagaaggtaaaaaaaatctacactcaacccattggcctcacatgtcagcctcctggcctcagtcacacagggattgcaaagctagaagtctcctaattcaaataaggaaacaaatccagataatgataggaagaggaaccgccacctgtgagtgctgactttgtgccaggtgatttacatatcctttttgatcatctcagaaatcgtctcaggaactgttccacatgagaaatttggcagtcagagaaaaatggtttttcagagttttccagagctaggccaccgggcaaagaggcaggatttaaactggtctatgccctgggtgga

SEQ ID NO: 78 - DNA, 2001 bp, Homo sapiens
aaattaatagaccataaattctgtaatgtgaatctttagacaaagactgctaaatctatcatctgatatatgaagttttattacaacaaatgtttatatattgagttaaaaattatctgtacccatacacacaacctgcatttacttaatttaaaaggagtgtcaatattaaaattgaggattttggccaggtgcggtggctcacgcctataattccagcactttgggaggccgggtagatcatttgaggtcacaagtttgagaccagcctggccaacacggtgaaaccccgtcttgactaaaaatgcaaaaattagccgggcatggtggtgtgcccctgtaatcccagctactggggaggctgaggcgggagaattgcttgaacccgggagacagaagctgcagtgagccgagatcgtgccactgcactccagcctgggcaacagagcgagaacccatctcaaaaaaaaaaaattgttattttgttgttattgttgtttttgaagacagagtctcactctgttgcccaggctggagtgcggtggcatgatcttggattgtgttcttccagggttgcagagagaagtgcaacagcgtctcattgcaacctctgcctcccaggttcaagcaattctcccgcctcagcctcccaagtagctgggattacaggagtgcaccatcacacccagctaatttttgcatttttagtagagatggaggtttcaccatgttggccaggctggtctcgaactcctgacctcaggtgatccgcctgccttggcttcccaaagtgctgggattacaggtgtgagccaccatgcccagcctaagaataataactttataactatgaaaaatgtgttacatattcaccattttgccagcattaatctcaaagcataagttttaccttttggggataaagagatagattgtgttttagccatatgaccaatgtagaaacaagctaaggttaactcctcatctaatgaataggcccgataaaaaagttggttgaccacaacatgaataaataaataaccataagcttggaaatgctacctgtagtatttcagagtgtcgatcacatttgtagtcatcacttaaagaattaatgtctgccaatgttatcatctaaaatgtatttacagaggtcagaagagctatgtctgtcctcctctatgcaaagaggtgatgacatcaaggggtatgtcacaagccaaagtttagttagcctaagttagttagctggtttgcagtttttatccactaagtctgatcttctgacccctcagttttacaactttccactctctgtgctgcatagcacctctccacttctgagttaatcatttctttgccattatctggcaagaattcttatctcttttgctgtcactttataacagggtcctcttatcaacctgcatagagtcatgtgatgagtcaaagcctatcaaaagtttccattcttctgattagaaaccatcatgaaactggatacatggtttacagcaggtcactaatgttggaaaaagtacagagtccagggaaagacttgcttgtaactttatgaattctggatttttttttttcctttgctttttcttaactttcactaagggttactgtagtctgatgtgtccttcccaaggccacgaaatttgacaagctgcacttttcttttgctcaatgatttctgctttaagccaaagaactgcctataatttcactaagaatgtcttctaattcagatactggggatttacaagagtctttaaagcacggacttacacctattggtaagtaggagtttctctattgatcatctatttattctgctactacatacatataatttaatcttgttgatggattttgcattttaagtcatggcttttcctattatgtttattaaaagcacacaataagcctatgtatatatatcacacatttatctttcttgcagttcaaaaaaagcaaatggatatttcaacatat

SEQ ID NO: 79 - DNA, 2001 bp, Homo sapiens
aatggaactaataacattttaactacattaaaattcatgaatcaaagggcccatttttgaatagtaggaagaattgtttatctaaataaataaatacaagttataacagtcatgaagcatttcttattgtatgtcttttgttaggtatttcttagttttaaatacttgggttagttccaaatctgtgatttctcaccttattcttaagatcctcaattgttaggtgatatctgctatagtcatgatcaagtccacggcaagatccaggtccgtatttttcataccaacccttgatttttctctctaattcagcatttgcctcctccagagctcgcacattatccaggtaggatgccaagcggtcattaagattttgcatggtcaccttctcatttccagagaggagtcccccttcgcttccagcaaagccaatacaagcagcatttccaagggcaccaccagcatggctcccaccaggaacactgcccaagccccctcccaaggcacaggaaaattcacttccagcaacagagccaccacatgcactgctgcctgcaaagcctgcacctccattgaggggtctgacagatccagctccagaccttaagcaaacatgcctggatccattagaaaattggagagacatggtgttttgagaaatgttcaccttgtctatgcaaaactgtaatgtcccaagagaacagaatatcatgcactgataatttctttgggtttttatatatcattattggggcgttcctatttgagttatacatgccaaaagcaaaatggcatcacttagattagcataaaattatgtataattgggtgattttaaaaataattaatgtctaacatccctcggtgttgctttgggctatttcttatcttaaatataatagggtttgttctatattaagtttattaatttaacttctaattttgagtgagtaatcctttcctttcatctagaccctaggcatatactgtcattatctggcgccaggtattatcaaattttttataaagttaagtaagattatgatagagttctgtgaggtacagtagggtctcaattaccatggaggggtataaaggactgctaattacagaaacagatattttatcccaccagaaaaaaatgtgttatttagaaaacagaggctagtgacatctctgttcttaccatgattataacatctatttattatcttggacaatgacacaaatattgacctattattttgtttaaaatagcatggttatgatgtttggtgctctatgctaagtgctaaaatgaaagacatagtgttaactaaatgcagaagtgaaaaatgcgtgattacaatctttaagaaatctattgtcaggtccagacaggggctggatcatggttagctttctgtgctggtctaaagagtttgtaatgaatcctaagtaaaaagcagagggcaaaatgatcaggttgaaatgaaacaaggtggtcaattacaaggttggaataatggtccaggtgagaaggatgaaggcagtggggatgcagagaagatggctttgagagcatatggtaatatgattgaatttggagagtagggaataaggcagaattggggataactcttaggtaattaattttagattctgggaactcataatattcactgagatggggaatagaggaggaggaagacactgagtttaggtttggaccaactggatttgaggggttgccagaatgttccagatgaaggtgactgatagcaagaatgactgtttagaatatgtatactgaactggaaaaatacttgactagcaatgagtaaaagaaaattggagggtccagctctcatttataatgatgttcaactgcatatttggaagtaatcctggtgcaggaatagaaaggatgatgatcagattgatgtgggttttgggatttgaagacaggtccagaagaaggaaatagtggcaaagaaatttctggtgttcttgagaaaacaa

SEQ ID NO: 80 - DNA, 2001 bp, Homo sapiens
ttttcatgcatgccacgcccgccgcccccatccagtcaggagggttggcctaaaagcttagtctgtatcgaaacacaaagatgggatgttgtgtccatcctcctcccccgatttccttcacagctgctccctgaagaagaaatgagatattgttttcgcgcgcgcgtgtgtgtgtgtgtgtgtgtgtgtgggtatgtgtgcgagagagagagagagtatgtgtgagagaaagataaagtatattttcatccaaaacatttttttttttgctatgtggattttctaatttaatttttttggtcagaatgcacttttaagttccagcaggacgtgttgcaacatgttttcaaacatctggtatttatttttcatttttgtgtaaatgaaaaatcaacaagctcatttcctgaagtggaacatggagttcctgactccactaatgggagtcaggtaatggcgcgagggttatgagtcaaagctggctttctccaggatcctgggcagccccctaacttctctgagccacagtctcctcgttgtaagtaggggctgtcattctttctccaccagagccagacgtgtaggggagataccagttctctaacaactggggctaaaagttgagaatgcagaagacatcttctgtcgctctgttctctgtcccttttatgacccttgcagcgagaagttcaagacagtctgtcactgcttgggggtgggggccggggtggtggggagtgtggacttaaacccaggggtgcttttggtccagccttatctctcctggtggaatgaacaactttgactcttgtgagtgagattctatgtctttgtgataggcaggaggtatgtttagagacatttagataaggcagtgccttcgtagtagacttagactcctaaatgaaattcattctaaaatatgtaagcaacagagatacatttacagcactgagtctataaacatgaaaacaacaacacagatccgtaacatcccaaacgttgtgagcgctttctttaagatgttctttgccttttcagaaacataagaaattcctactaccacccagggcctgcctctcgctgacctcctgtgctgccctcccctaagagctctgtgtagctcccttccccactccaggtccctgatcagatgacacttttccctaaaaagcttcctgacctttcaaaaatagcactcttgtcactgtccctttccctgcttcattatatctttacctgaaattctctcctttatttatacattatcctgcttagtatgttttccccaggaggataggagcttgaggcgtgccaggagtttgtctctttgactctgtgttgtgccccagtggctaagcacatactaagtgctcagtaaatacttataaattaaatggatgctatccaaagaacttaatgagtttctaagatatacaaaaggccccagaggtaatattcacgcgattttgatttcaattgtatttctggagaagcactagcctgtatgtttctgtcttcttcacctgtatcacatttgccaccaagaattgcctgtcattgaagaaatacacagttagcctcttttcagcctattacaaaaaattctctctcctctaagcagttgcacagttttccacaaagcactgttgcctaagtttccaaatatgtatgttggagttggagatatctatgtattttagccctcagcactgaagtcggcaaccatggagctgaaggtccaggtctaggagtcacagcgggagaaaagccacacgatcttcctaggtctcagttcattttttctgtaaatattggtaagtgatatattccttagggaaatcctctcttcacatgtttctggaatgtatagccagtaacaaattcttaagcagcataaaacaaatcagacctatcttctaagcaagtgtttatttttaagtcccaaaataaatacacatttgttcaaaacaattaaaatagcaccaaaatgcaggactc

SEQ ID NO: 81 - DNA, 2001 bp, Homo sapiens
ggtagagttccagaaccggggagatagcagcaaagaccctcccagggtttccctcatgaacctcagttcttgggaggctcccccttggacagtggcctttatctgttcccagggaagctgtggcttgggcctggagctggaggtggaatgatctttctgggagcagagaggcccagagagtgtgcatctggggcccctggggactttgtgctgaaaggagagaaagttagtgagatttgggtcacctgggggcttacccactacaaagagagtcacaagagagaagaccaaggtcacagccataaatgccggggtagcctgaacgagcctcggtatcttgggggctgcaggagccattgccacagagtccacctctgcaggggagaaggcagtgtccaaggtgagagggtgctgtgcaaggtaagagggtgctgtgcaggctgagacacagcctctctgattctgtttcctgggttgtctaatggggatgataactcctgccatgcttatcatgcaggtggttatggagaaggaagacaaaacactttgggctgttcaccagagctagatgtggatatggtagcaacacagagctgaggagctctgagaccgagaccaccgatctccaaatctcaaggaggctttggtggtgcacagggcacagaacctggcacagagcacagagcaacaaaagtgtagtttgaatgaaagaagaaaggaagggagggaggcctcacaggaccagctcagtgtgcagagccgggattccaagccaggaccatctgaattcaaagttcatcccctttctcttgccaggtctgcatccccaaggacaagggtctggggagagcaataagacagcaataagctgccttatggcagagggccaaaggacaggcaggctactccctcactgggcaagagctggggccccagttttctcaccttgggggtgcagggagacacactggttatctgtgcagaagcggactgcctcaccgtccatctctgcttccttgatcctccagcactgctcccagccactggctcctggaagggccgtcccgtggaccaatggcagtggaagcaaagctgagacacccacacttatatgctcctgaccaccctcccagctcttcctccctccctgcctgactctgcagaaacccgccccagtgggcccctcccctagaggctcccagatacccatctgccctattttcttcctcttcccattagcaccttcccgtcagttctcagccccatcatcccagcctctgccgcatccagcataaccccaaacacacctcttcttctctgggggctgggactggcacctaggaagggggtctcagctttgcacagaaaacatccttatgactatggctgacctttaattctagcctcagaacctcttaagtaaaaggagtcaggtaaaagaggggccaggtcagtgcggccgatatttgcaggtcaaaggaggaagcggggtgaccagccacttggggactcacaactgaattatagtatacaggaggatgtgcggaggttatataaaaatactacatgattttgtacaagggacttgagcatctgtggactttggtacccatgggagtcctgaaacaaatcccctgcagataccaagggacaactctgtaaaaaatatatctggtcttcatctcagttcctgacacagaactcctaaaacgcttagagtttcctgggtgacaggagtgttttttgttattcataataagccccttaggactacacttgggtttatgctaatgaggtgacacagggtggacccttagataatttcaggctggaggctggtcccagaaagaccaaacatgggattagactttggaagactgggactttcagccacccactcccattccctactcccagacctccaggtatgggagatggcctggggtctgggctataacgactcttaaaaaatgagattcaaggagcttagggattggtgat

SEQ ID NO: 82 - DNA, 2001 bp, Homo sapiens
cagtgccctggagccggttgggttgagcgcccctgcccggggaaggtagcctgcgggctcttgccccgagcccgcggagcaggaggtctcttttccaagcgcactcacattattcatgcaaagttaatccccgccgcgtcacggccgcccgcccgaggatgtgcgccttccttggccaagccccgcagtctccaccctcctccgccccctcctcccggcggcggggtcccggggagcgccggcgactgggcgtcgcgcgagtcctgcaaaatgtcaactccttgggcgaagagaggcggccgcagccagggccgcgccgcacctccccggtctctccacttttataggcgtaggaagctactgcggctggggcaccagagtccccgcctctccgggtcccccgcgtgcccggcccgccccggcccgcttcccgggcgctgtcttactccgggcccggggcgcctgctccgcgccgcgtctgcgaaccggtgacctggtttcccctccagccctcacggctgtccgacttgcgcggcggtggcggcggcggccaagagcaggcaaacccggctccgccaggggcgcagcgaggaaatggcctcctggcgcacaccccgccgccgccgccagccatcgccaccgccccgggctgcccagccggtacagacgccccctgccaggctccgggccagcgctccgacgcgctcacagcgcccggcttgccgaccccgaccccgaccccgagtcctacgcactcgaggtccccgggccggccgcctccgcacctgcctccccaggtgtgggcgctccccctcctcctaatcccggccgtgccctcccagggttggtgacggtgctgtgggtttggtggccgcgcgtccacccacccgattgcctgcgcgcataggtgcgggtttgggcgtcattactctagttggagaagaggaaagaatggggaacgagtgacaccgggaccggagggcgagtcttccaggagcacgtctcggccttctttgcccggcccgaccggcccgacccgtgccgcagcgctcctccctccgctcctaaaccacttgtggcgcgtgtcgggaatacagggcgctctggctcttcttggggcctccgctggtccgaccatctggcagctctcttaaccaccacggccaccccacgaatggaagtttgggacgtgcctgggttgcccttaaggaggacacccccgcccccaccgccattatgcatatcgtgggagaggcgcgtacagggctgagacactcctcaggtcccagaacggccgccctattaatttgtctttgccctggaaatgtatagataaaaccaggaagacaaaaataatgtatactcttcaagagatctttcccgggcaaatataggtctctcaaatgcccccgaccttaagtcctggagaccaggaagagacttgctttctgagctagaaggatacttgggaaataccgagtcctcactcagctaaaaggtagtcaataaggaactatagttgcaacaaaactactttgacaacagagtacaattaatttcttatagacagtattaaaataattccgacatcataatttaatattcacatctattcgtgtaacacgcagtataatagagttatgacgatatggatcctcagctgcctgacaatgtataggttatttattggaaggttctctagatttccagccagtgacatgtatgttttattctcgcttcttctgagcgtcccagggaaaggggtgagaggccccccacccctgctgacagcattaacaaccaccccgctggataagcagggtttcactgatgcacttttaagaaagttgcaggccccagagaccccatttccctttggtcctcggtgagaagtccgtgctttcttctctactcttaataagtctgcaccataggacttgcaggcttctgtagttggagagctgttctcctttgcagggaagttcctgggctttgctccaa

SEQ ID NO: 83 - DNA, 2001 bp, Homo sapiens
gtacacactcacatgcatacacacacatatgctcacacacatgtacacacacacatatacacaccacacactaacatacgtacaggcgcacacaccgctacacacacacacatgtacacacacacacatgcacacactttggatcacaggctcaatttctagaccacacctggacctcgttaaatgctgcgggagttaagtgatggaaactgacccttccacctgtatgggtctgcacgtcattccctctgctgcctcaaaccggcagccatcctgtctcctcagcagactgggacctccccaagggaggacagggctggaggcttctctgggccgtctcaggcctcacccaccttgtcgatgaaggccgcgaacctgctgttgagggacttgatctgctccttctcctcctgcttcacgcactgtgcgttggggtcgatctccaggttgaggggcgtgaggaggctctcgttgaccgacacggtagtgatgcatggggggctgggtccgcacacgcccccggagcggtagccgaagctgcgtccgcaggagccggctcggaagccacctacagctatccggggcccgcaggagcccaggttgcagaggctgcggctgccgaagcccgtcagccctcggtagcaggacacccctcggtagggggcggcgctgatgcagcagcggttgccagttttgggggccacagctgagcaggagctgaagttcctggtgaccccgcatcctgagctgatcctgtaggagcggcacgacatcgtgtgaggctgagcagagtctgagaggcagcggaaggtggtgcgggcggcagagtgcgaggctcaggatccttctgctctctctgatcctccctggtccttttattggtggggagagctggggttggctgcataaaaggagtggaaagccattttatgttgtttatgggcttggggagtttgccagcaactccccacggactcatcttaaaggtgagctcagcagcgactctggggctccctgaagtcttgaagatgttattaaggagactgcatttgaacttttcatcttcaaagctcgtttacatacattaagtaattaattttctcacagtctttctgtgagatggcctcaacccacacttggagacggatttgaactccacaacagcctgggggtggctgggtttgggatacaggcaggagccattgtctcaagggagaggttggagggtcctgggttaggtgagacttgggggatcattgcattcatctatctggttccacagaggacacattctcaagtaaatcattgcatttcaggggccaataggtcaggcgaatcccaccccatctaggcagcattgcttggaaggtggttcaggaccagagcggtcattttgaccatgcccgtgccaggataaaaggctggggcagatgatctcttggaaccttctggattccagcccagaaggcgctggtccagagctttcacttctcacccagcaaaagtgcaaaccacagaactctcagtggccacaataatggctacttgcggattatggggagactccctctagccactttcttccattcggtcaaaaggattggagaactcaaccttgggataacctgggggctataaatttgctgtgggcacctttgctttgctctttaagaagaaacaggtcatgtgagtctcccttcccagaccaccagctacaagaggtattccttctgttggccagcccttgaattgtcatccctagaggagtgtttagagactgcattgcacaaagctggaagtgggatagtggtttattcttgtctaaggtggctatgacttctggctcctgggggttctgggagccccatccttctagtttctgcttccctgtgtgtccttgggacacttattaggataacaagtgctattcttttaatgtttatggagtacaaaccttggggatgtgggtctgcccccaggaaggttagccagacacccaag

SEQ ID NO: 84 - DNA, 2001 bp, Homo sapiens
tgccaggcactgctccaaataagtgtttgaagagtgtttcttcatttcattctcacaacaagcctgctactatacacatctttcatatgagaaaacagaagcacagagacattaagtagcatgcccaggtgacacagtttgtaagtggtaaatctgggacctgaagcagagaatctggctccaaagcctgtcctcttactaccaagtgacgtttgtgcctggcacagagaaggtgctcattaaatgtttgctgattcaaatcattttaccaagaaattaaacatgcttgagcttattgttctggacttaatgccctgactactcttaaaaagcaaacaaaaatgcttacaagtacaccacaatgaaacaaaacctggaattatcagaacatggtaaatgctttattagattttctgattgaccaagggaaattactctaagctttcaatgtcttcttttcctacaagggaactaaatttccaggaagttctgtgacaaagccacagagctgagaagtaaattagtctgtgctggacctaggatttcccttgttcttccacctctgtccccagcaattgctttgctgcctattctcaggggaaggcattgcactagccatgcatgctccctggagaatgacaacttgaactgcaaaatctcaaaggttgtgcttcttttgttcttcctctcatctagttgataggcagagaacaattctgattgaatattctatttaattatgtttaatcatactaagaatcaaccaatgttaattattaaaaagggagaaaatggccaaaacatcatgtacaaccatcctgactttaggtatgaagggtcaacattcattcagctctgagccaggcagttaaacagcaaacagttttaataaaggacaattaaataagtcaggaaatccaccagtctgttctccaggttcactcaccttggtgtggaggacgatgggcagaggtagtaaaaccaacacagtgaaaaccacgaagagaaatcggcgataaaccagaatgtaactgaagaatttcattgtcctgagcaggtggcttcaatagtgtcctccaaataaagcagatgttaaataaccaacttggattaatatttttccagcctgtcaccttcactcactgccctagaccgaccaacacaagttaaaattaaatctcattttttattgttttactatcacaattaacatgaagagataggcccaagacttttcatgagaaatcaaagaccatttgctcctctgggttttcaaggaaaaactgttctttccctgctttctttgatcacattcactgaaatacagactggacaagcctaggagaaggcaaaaccatgaagctggttgtttgttcacaatgaggcacttccaacccttttgatgtcctctgacttccagactccctaactggagtagttaccacattgtcatggtgccatgagtgagtctccacaggcagcctccatggccctttctaaggaagtatgttttcagtgtgtaaattgaaatagcaactaatagttggaatttcagtgtatgacagtgaaagggaaacttttttgtttagagatggatttttaacggacttcttggacaatgtattgcaagggtcttctacaaaatttatagcaaatgtccctgttggttagaagcaaagttttgtgtatttcttggaccctattgtctcttttgatagaaaaccaaaataaccatgaagtgttatataaaccctctattttaagacctttgaaggcttgatttgatactgttgtgctgatacagactacataattatcagggaatctgcataaagatgagtgattagggacattcaagtggtgtgtgatgtgtgtgtgtgtatggtttgtgcacatgagcacatgggaagttggagactgttccaacagtgatatggaggatgtattttgaaaatcttcagggataaggaccctggtttttttcacgttgtgttgtaaatt

SEQ ID NO: 85 - DNA, 2001 bp, Homo sapiens
ggagaattgcttgaaccgggaggcagaggtttcagtgagccaagatcatgccactgcattccagcctgggtgacagagcaagactccatctcaaaaaaaattaaaagacaaaatgtggacacacacatggccccagcaagcaagattgattactaacttcgtggtggcgtgcacatgcacacgatgttccacaagctcatcatctattcgagtcgagagtattcagttatcagcctcagaaatccagaaggaacctcacatcccaggagcctctggcatttcagggcctttcactccaaggagccgcctccaccgggccttccccagcaggatgtatatggccctgtctgtgtgctaggaggagaagggcaggcccgtgtgctgtctggtgctaggtgtgagaccacagcctttctggaaaggctttgtactgtgctgctggtgtgcagaccccgtgcgtggtaggggcttcaagaatacgcatcaaatgaacccgaacccaaggacgcttgctcacctgtgtcatctgggggtctccttccacaggaagcacatctgggcagagtgaaggctcccagggctcaggtccagaaagagctggaggctcctgagagcaaggggccctactcggccatgagcccagaacaatgcgagcttcacacaccgcaggtgtgtgcgtgtgggagcctcttcctgaagccttggggtcaggggccggggcccagcaggctgggtttattgtccattgtctcaggccgggacaggaagcactttctgttcccaattgctcgggtggattaatttacagtggggagctcagagtcgcccacggcacctgttttcccctctgaggggccagcttccagcttggagggtgtttcaggaggggtctcctctgcagagacgtggcctcctccagagacccttggccacagcccctgcccgctccgtgctgggccgaggccaaggccctggcagccctgcctgcttgcccgggtcacgctgcccacccacggggaaagctgagccctgtggagtgcacaccatgctgtcagcagtgagccatggtgaggcagcctcagcaatgtgtcttctctttggttgggaatccgctctctcttccaggttcaggggcaggtgatgtatgcttgttttcatttggtgaagatcgtgagtcccattttacagataaggacgctgaggctcagcaaggttgactacacaggtcgcgaatgaagtggtgggatgcaaacccgagtctgctccaggtcccagagctctcaacctcaaatgtctcaggagccaacgaaagcttgtgactgagtgtcaccagctggtgtacaaagggggagtggcaggaggtggaggagaacccaggggtgcaggcaggggggaccctgtgccatctccaagggcagcactgctctacccagcctgccatgccttgaagtaaggtggaccttgtgtggccacatttttccttgttagtaactcagaagatacatttctagacacggggtaggtcacaaaagcctgtctgcaggcggcacattgctttccacctctgctctgtgccaccatgccaacaccaacagtgagcacctgctatatgcaggtgctgcccgggcacctccacaacggggacccatcctgtgtcttcaggcccccccaggcagcaggcactgtgcttcctgtggacacacaggggactgaggctcaaaagagcaggctcctgaccctgggcaccaggctggtctcggcttgctcactccacccctgcatccctgcatcggtttttcatcaccactaatgcaggaggctgaggaagaattctggagtctggtggaggatggggaagctgaccctatttctataggtacctccctcatttttgtaggatggctccaagtcagccaccgagactgccccctctgcactaaccattagccagcactaccaggattggccagcttgctctgtgccacttgttttatttatttg

SEQ ID NO: 86 - DNA, 2001 bp, Homo sapiens
gaagtggccctccccaagatgttcattggagttgccatggtgatcactatacacagggctctgttacctcagagggctgataggcccacccgtagggaaaagaggccaggagaaagccaggattgaggccaggaaaaggcaggaggggcttcaattattgttcactctctcatccactctagaatgtcagttgagcccccactacatgccaggtgtgccaggcacagggggctcagatgctaccaagtgcaaaagatgttcttgggagctcaaagcactaagtagggagaagaactagctctgtgtaggaaaaaggaggaaggacttatgaaagagccatttgagttggatcttgaaggatgcctaggaacttaacagttttgaagggatgagaatattaaaggcatggatgtgtataacagcataaatgtaggtcgaggtcaatggggaaggagaaagatgcatctggagctgtctgatttggacacccagggatcagggacctgggagtaaaactaaaggaagggtcctctctcctattcctaaacaacaagggctacagaaggagatcaacaaaattaagtgacttcttttctgcagagaagaaggaggcagaagtcattggtgccagcactggcccattcctgtctggctgttgctcagcctggacaaagagtgggtatagtgaacgcctcctgcaagcccagcctttcaggatttccccaggaaacagagagtgctgtggttaaacttggagtccccagaatctgggcctgtcacttattagataagagactggccaagtcctttagctgaacttccttcacatgtgaaacagatatcacccaacactaaggataagggtgatgggcgaataaaacaattttttgcaaaatgtagcacattgccttgcacagagagagacatcacaaaactctaagctacttccgcaagcaacccatcagcaagtccttcgaagtacactagatttggatgccttcgtccacctccacaactccccctactccctggtccaagccaccctcatctcttgcctaagtattatctattgctgcacgacaatcttgccataaacttcgaagcttaacatacaactgttatctcacagtttctttgggtcgggtgtctactcatggcttagctgagttctctgcattggggtcttagcaggctacaatccaagtattggccagggctgcagtctcatctgaggttccactagggaagaatatgcttccaagctcatgtggctgtgggaggattcagttccttgcagtctgttagactgagggtctcaggctctctctggctgtcagccagaggccatcctcagttccttgctatgtgggccccccaacaccaacatgacactgacttcatcaaagccagcaaagagggagtccccttgcaagatggggcattacaatgttgtgtaccatagtcacagaagtggcatccatcacctttgccgtaggctgttggttaaaagcaagtcacgggtcccacccgcagtgaaagggagagggtcacacggggcatgaacacgagggggcagataatatggggaccaccctggagtccaccatatcctctgacagtcttctcactcaacagcccagcaatcctgttaagacctatattagatcatatgtcacccctctaaacaaaaccttccagctcaaccacagtaaaagctgacatcctcccatttatctgcaaggccctacccaacctgtccaccatttttctctgacctctgctcctctcactttcccaagtgctccctgggtccctgccatgccaggctctgctgttttttgggcacaccaagcacagtccctcctcggggcctttgcacgggctgttccctctcctctcctgcaacttccttcccccacatgcctacatggcttgcctcttcacctccttcaagactctgctcaccttcccagaccaccttttcttaaaaagcatcccagc

SEQ ID NO: 87 - DNA, 2001 bp, Homo sapiens
gccactgcaccccagcctgggcgagagtaagactctgtctcagaaaagaaaaaaaaacaaagaaactttaaaatagtttaatgtcaatgctactttcaatcttctctaattctaagtcctacacctctggtgaatttaatacttttaaaaagctgagttatgaaacctgttttattttttatctttttctattaaagtgctaaaacctttcagtctattcacagtagcagattttcaaaggagatcttatgcttggacaaccaaatgcgctgatgctacattataacttttcatactttgcagattttcattcattataaaaataatacaaagcacttaatcttttgctatatgtacttattctttccatttataaaactaaagaaacataaatctcttacctatgtttctatctgtattaaagctgttagatctacaaattttttaaaagaaaagaaaaacattactcattagtataatcataaaaattaacatgtaagatgatataagtgcaatttttaatggcatactacagttaagtccttcctccaggtaggaagaacaaagaaattactcattacaaagtctagtaaatccaagtctacttagagaaaacagaagtacttacttcgctcaaattaccacatttacccaaatttaagggagttgttttttccaaaaatattaaccaagagagaacagtgtccaccaaagtctcttgtcaattattttgctgacattagaagacacagcctcaatatataaatatattaattcatcattcagtaaatacatactaagctcttatttaccaggcacagttctaggtaataggattatagagataaacagaacaaagttccaacctacataaatctaatgaggaagacagaaaacaatgatatataacagatcatgatggttttcagtgctataaaaagagacataagagtatagaggtaaaaaaaaaaaacagagaaacctcaatcataaggaaatacgtgaagaaatgtagaatgaattatgcagcttactggggaaagtgagagaacagcaaatgcagaggccctgaagcaggagcattcatggaatattcaaggtacaaggatggatgttcactcaatgctcctttgaaatgatatagaggtcaagagatggaatcaaagaggaggcaaagaaattaacagtcattattcctgggggcatgacctcataattttaagtgctggatgtcttttaaagagtacttatgaaaacactgtagtgaaagtatgtatcacagtgatcataaatttagagccacaaaaaatgaaagccacaactgtctggatactatgtcaattggtacctgtggctatagatggtttccaaggattattcacctacataaaaggaccgtaaatggaagagaagataatttgatctggaaaactgacaaagaggtcaacctcaaaaatgtacatatatgtttcatgatatatggttttagaacagcagtatttctaaattaatagatatcatacatgacttcaaatatgtatacacacccaaagtaaaaaatgagcagacatttgaccatctcacctacatccctataaatatacttattccatttggctaaagcatttttagaatcaacacatggggaaaaaaaatggtaactgcttcctatttggtcaccttatactagagcttttaagaaatactcagtaacacaagaacagaaaaccaaaaaaaaggaaagaaatcatcaggccaagtgtggcagctcatgcctataatcctagtgctctggaataccaaggcaggagaactgcttgaggccaggagtttgagaccagcctggccaacacagcaagacccatttccatgaaaactaaaaataaaccagacatgatggtgcatgcctgtagtcccagctacttgggaggatcgcttgcacacaggggtttgaggctgcagcaagctatgactgcaccgctttactcactacagcctggg

SEQ ID NO: 88 - DNA, 2001 bp, Homo sapiens
tggggggcatctgactgcttccccccaccctctgccaggcattgctggagaatgttaagacggcgatggagatgccatcaaccccaccctgcagagcatcaccagacaccaccagaccaaattcactttccagccccttcatgttgaacctgaaacttgagctagtgtcttgggagaaaagggggaaatctctacgaggtacccatccttctgcaccttaggtctgaggtgcttggccccctaggaagccctacatgaatgggacagaaggtccttaacaacactggagatgaagcagccgatgctgttttggacaaatgaaacagcgtcccctaaccagccctttctatctcattgttctgacttggacacgccatggctcaccgctcccaaagtccccactatgtctccctagctgaggaaataaaagcagagaggggtgatgaaacagtgacgatcctggggaaacagctgaggaggggagggagggggaagaagccactaaaaaagtgaaatgtgcttgggagaatcggcctgcctgcagggtagatgccctttctctctgctggccagctctgcccctcagtgagaaactttacatattgctaagatgcctggccaatgaaacagttccagagactttatgtcccccagtagaaatatgaatagaaatcaccctgtgggcaatggtcccattttaaaatatgctgtcccattgtcccctagagcctactttaacttgtcagaccatgtattccacttcatatgcaagaggcatgcactgagcccataggtggctaggcaaacacccaatagctccctgaaatggcttcattatgcagcctcgacagccaccccaaccctcccactctcacactgaaacacccagacctagagatagctagacacacccagacacccgccaagcccctcacatacagatatgtgcacaatgatacacagcaaacgtacacagagttcagtacacacaaagagctcacgcccacgtgcacacacccctcagttgggacagagttgaccaccaccacctttctcccaaacacatggcttttgaactgcctttccttggatccagttcaaggggatggaggagcagtgagagtcagccgcccttccactccaatttcccagcacctcccttatctctgcctcacaagtcacccagcccccctctcttccttccttgtgcttgaagaatctctccttgctggaaagccccctgttttctcaatctccctttccacttcggtaaaatctctacttgctggaaagccccctgttttctcaatctccctttccacttcggtaaaatgcccactttctggtccccacctttttcctgagtgtagtcccaaccagccaaatccaacctcaaaacaggaagacccaaggccagtgacccccataggcctgaggcttgtgcaggcagtgggcgtggggtaaggcttcctgatgccccctgtccctgcccagaacctgatggccctcattagtccttggctcttatcttggaagcacaggcgctgacagccgtcccagcccttctgtctgcgggcctgaaccaaacggtgccatggggaactgtctgcacagggtgagtatggggccaggccccagagtcccttatccctatgcccctcatttcccgtgctgtttgcccctcagtctttatatctcttccttttcctcctcatcttttctcccttcctgcttttttcctcttccttcaaagtctttttccttctctccttcctatgctagcctcctagctccctcttgtgtccctccctttgcctttgagtcagttccatcctggtctcttggtgccttttccttctgaccttgcactgctcctccagccccagctgccctggcttccccaggactgttcctgctccggctcttcaggctccctgctttgtccttttccactgtccgcactgcatctgactcctgcagagaccttgttctcccacccga

SEQ ID NO: 89 - DNA, 2001 bp, Homo sapiens
caggcgccctttgagccgggaccagaatggatgagagggagcagctgtgtaagcatcggggggaagagcatcccagacagagggactcattgcttgcctctgcctctagaacaggcccttaggaaaatcccggcagtcgcctgtgctcagggccaggctttccatggttgccacaacacccagggcagcttggaggggaactggcctgagggccaaagtctgaggagcaggtgggcagggtctctgaccatgtcctagagccaaggccctggtgcatgccccctccccactgccccctccacccctcactcctgttccacatgtcctcaccccaaccctcacacttagctgcctggctgcccacagcccgtagctctgcacatccaggaacaaccagacagaagggcaggattcgggcattcttcagaggccatctcccctcgatctttaagggtaggggaagcggaggggagcaggggtggaagggagagaggcatctccagcaccatccgctctcccagcattcgctcagccaccctggcccatgtctgagccatgaggtttgagcatagctgcttctccacatggctctcccaggcctggttatcctgaagaaaaaggaagcacgctacccgctccttgctaaaagcattccacggccttcacctccctccacaggtctgcacagctggtgggagagggacagtgacaggttcctagtcttggcacttaccccctccctggatggaatgggtggaggggttcccgcctctttgttctccatccttgttcaggtgggagaacaggtgtgctctacctcccaagtgacacctgctctgcgccagccacctaaagaccggctgtttctcccctgggcctacctggctttatgatccttttgctctctggaccctgctgactttccccaggagcaaattctccttggaaatgttgataagacacacgttgccagtgcacaaatcatacacagcacgaatttcgtctttgcacccaccaagccccaagccgaaagctccctagttgcaccaaggagagagaaaaaacattgcctgacattggtccgcagaaatgaggactttcagagacccagtgaggctgtgacctggatctaggatgctttttctgtgtcatttttccatgtatgcctcttttttttttgagacagggtcttcctttgttgcccaggctggagtgcagtggcgcaatctcagctcactgcaacctcggcttcctgggctcaagctattctcctgccttagcttcccaagtagccgggattataggcacgtgccgccacacccagcttttttttttttttttttttttttttgaggtggagtctcgctttgtcgcccaggctggagtgcggtggcgcaatctcagctcactgcaagctctgcctcccgggttcacgccattctcctgtctcagcctcctgagtagctgggaccacaggtgcctgccaccatgcctggctaattttttttttgtatttttagtagagacggggtttcaccgtgttagccaggatggcctggatctcctgacttcatgatctgcccgcctcggcctcccaaagtgctgggattacaggcgtgagccaccgtacccagcctttttttgtgtgtttttagtagagatgaggtttcaccatgttggccaggctggtctagaactcctgacctctagtgatccgcctgccttggcctcccaaagtgctgggcttacaggcatgagccaccgcacctggctttttttttttttttaacttttattttaggttcagggtatatgtgaaggtttgttacataggtaaactcgtgccacgggggtttgttgtacagattatttcatcacccaggtattaagcccacttcccagtgtttgtttgctgctcctctccctcctcccaccctccagccggaagtagatcccagtgtttgctgttcctttctgtgtgttcctaggttcttcatatat

SEQ ID NO: 90 - DNA, 2001 bp, Homo sapiens
tattttcttgctgtcaggtagttctcaagttgccttagaaataggggtcacctctgaggacagagagaagagaagggcaaagagttagggcagtgctttaggtctttagtgtttatttctttacccagagaagtacaggaaaaatataacaaaaatgttatcattgtaaaatctaaatggtgaacatttcaatgttcgccatagtacttacagcatttttagtacatttatattttgtaatttaaatttttggggttttttttgcctcccaagtagctgggattaaggcacctgccgccacgcccggctaatttttgtattttttagtagagacagggtttcaccacgttggccaggctggtcttgaactcctgacctcagatgatccacccgccgcggcctcccaaactgctgggattacaggcgtgagccaccgtgcccagtcaatttaaatgtttttaatttcacaacttacattctagcttcaaaaatatattttacattattacgaaccactcaccaagattttatggccagatacccaaattcatctcagtctccaagtgcaaattttaaaactaatttcttccatcattcaattcaagcagaatggtagagtagtattttagaacagatagaactagtaaatcccagctctgtgaacatactttctctgttaccttggccaagttatttgctaaaggcatttgcttcccatcctgtatctgtaaaatgggtatttcctattgcaaaaggtagataagtagcatgtgcttggtgggttagtgtttttccctctccagctggaaaaaaaaaacctggtgtgggggaagaaggggaacagtttgtgcaacagaaccctaagaggaaatcagctgctcatcaagataagggctgaggcataaaactgccagagggtctcaaggcaggcaaagaagaaccatgaggctcatcctgcctgtgggtttgattgctaccactcttgcaattgctcctgtccgctttgacaggtaaatcttacttcctgtgttccagtctctgtgtagaagagaattaaaggttgtttccttacagttcactttttttttttcatttgcctccacaaagggagaaggtgttccgcgtgaagccccaggatgaaaaacaagcagacatcataaaggacttggccaaaaccaatgaggtaagcatttagagggatattttatcttttcttactgttctctaaaagatgcttcttcttattttactgtcaatgcccatgggactacaaaagactattgaacataaggggaaagcaggagctatctggataattcagccaatggccaatacttcacctgttctggttttggtcttggtttccaagactaccacttctgtttttcctcccaaaagcttttcaaacaccagctttttcaaggtaaaactatgaaatcaggagctgttcttggcctccttatcagggcatctttctcttgcctacgggtgccattccctctccattctccccactccgaccctcatctcctctggtgccataatttctcttgagtactaactgaaaacgttgaggcactggctaatatcaagatgtgaccctttccaagagcaggtacattttcaaataagacttttaaagttagatcaatcctttaaaatccaacagcctaatataattttatcatgtattcagctgctcctatttatgttgcaccagccaccttatatatgccatttcaaattctccagcagattctcaatgtcagggttataatgtaactcatttgaaacagaagagctaaagtgcaaagacaataaattaaatgaccaagattacacatcaaatttaaacccagaactgggtctgggatttattccagtcaaacatacagttacctctacatcaaatcaatcaaaggccctgcctattctaactcccaaatgtttctcaaatccacacattgccctccattccactgccactgccaagttcatgtt

SEQ ID NO: 91 - DNA, 2001 bp, Homo sapiens
cagctcaactcgccgagatgacctgggcacctctgcgttgaatcggcaaatactgatcaagccgcatttattctgctctcaggaactctaagtctagcagagaagatgaggcggtagaagttcatcagtcagtgcccgaagagaggtcctaatgggaattcgaagaaaggagttaatcaaggaagtgtatttttttgttgttgtttgagatggcgcccacctgtaatcccagctacttgggaggctgagctaagaggatcacttgaacttaggaggcagaggttgtagcgagccgagatcataccactgtactccagcctgggcgaaagagtgacactttgtctcaaacaacaacaacaacaagaaaattagccaggcatggtggcctgcacctgttgtcccggctacttgggagactgaggtgggaggattgcttgggcgcgggaggttgaggctgcagtgatctgtgatcgtgccactgcattccagcctgggcgacagagcgacaacatgtctcaaaagaaaaaaagaaaaacagaaagaaatcctaccttacatatacagccaaacagttataaccactccatatttaaccattacaaacgtaaatttttattttattttattttttgagacagagtctcgctctgttgcccaggctggggtgcagtggcacaatctcggctcaccgcaaccttcgcctctcgggttccagtgattctcctgcctcagcctcccgagtagctgggattgcaagtatccaccatcacgcctgggtaatttttgtatttctaggagagatagggtttcgccatgttggccaggctggtctcgaactcctgacctcaggtgattcgtccatccggcctcctaaagtgctgagattacaggcatgagctatcgtgctcagcctacaaacataaattcttaagattggctgcatcttatcttcccataagatttgactgtactgcttattgcaaattatttcataaagtgcgtcctaaattcacctgataattgaagtagcagtttgttttagctaattgcctttatctgaaggaataataaaatttctcttatttctatgacaagcaatatcatttcatctcagaaaaagatgcctaggttgaattcccacaatgacaaggagataggggttttattttcctagatgtttatatttgagaggaattactcgcagacccttgaaaaaacatgtcagggttataaagctgacaagggacttactcatcttctaattgggtttttttcagatggcttggctggaggacaagcaaattgaggacattggcaacggagtgatcaaaatgatagatcatgaggcctaaaatgaataaggaaagaagagaagtggcagaggctgagaacagaaagagagggtggaggggctgtaaatcttgaagattagggtataatatgagtatatgggtaagaattggaagaattgtgtaggaggcagtagtcaaaaagtagaagcagtttggaagagtagttacaaatatcaagagccaggtggctaaaaggtggagctataggtcattgaagctcaagaaactgagtctctagggcattggttaagtcatctgtctagacttcaaagttgtctaggatgataattcagaagactgatctgtgccaaagtcacaggtaagggaagtgaacaacgaggaggggaaaagggagggtataggctgatggaaggagcctcagagaggcaaagagtttatatcctgttgtgggagtggcatcaggtataaaggaacacaaactccttattttttattgatttgcttctttgtttgctatctgtaatgtgattggttacttcctgcagaagattttttttttccaaatgaattatttcctttttacatttgcaggtttttcacgactgaaaacaacatagcaaaataagccaagatgtctgtggatccaatgacctacgaggcccagttctttggcttcacgccacaaacg

SEQ ID NO: 92 - DNA, 2001 bp, Homo sapiens
gcaacttggccgaattttgataccccatgtgggggtatgaattcttcttctttttttaattactcaactccattctctacttttctaagcctgaatagatgaggccactcagcctcagaaactatgaaaacttcataaggaacaaaaggatctcaaagaataaaacctcttaagcatataccagccaacacctggctaaagtcccagtgtttccagcctgtttcagagacatttctaagtgaccagatgcaacatcttagtttgtcgagatgccctcgagtttggctggatgctcctctgtcctccagaagatatcaacactgtcggaatctatcccaccatgcagctcgtcctctggcttggccttgctctgcagcagagcaacccataaagcataccacaatgcctgaggtaggcacttgaaagatctgaggtcaaggtcaagtccccagaaccgggccagttctttttaagggaaagagcactctagcgcattttggctgtaatattcctagacccagagattcagctatcataggtaccttgtcaatgaaggaggcaaacttgttgttgagggtcttgatttgctccttctcatccttcttcaccctctgggcattggggtcaatctccaggttgaggggggtcagtaggctcttgttcacagtcacagctgtgatagatggggctgctgggactccaacccctccaactctgtaaccaaagccagggccaccaaagccatagccaatgccactgccagctccaaagcccagaccaacacagctgtcagccctaggccccagaccaacacctctcccatcaccaaaacccatcccacagccagcaccaaagcggactccacagtggatgggccgagagcctacagctgctatccggggtgagtacgatccaaaggtgatgacactccgactaccaaagctgccaaggccccggaatccaggcccactccaacaggagacagagttggcccggaagcgattcaggttctgtggtgtcattgctgaacaagagctgaagttgcccacccggtgaccagagctgactcggtaggagcggcaagacatgatggcttcctggttgggagcaaaagagcaagtgtagaatgggtgagctggagctgggagtccctctgaggccagtggaacccttgcaccccttttatgtgtgtggtgatctgaggcttggccccataaaaatgaaaaaatccatttgcaggcatttatgagcttgggtagttagcagaaactctgcatgcctataacctccttgacaatgagttaactcagacacacctagtctcattgggatgagggctgtaacagcaaaccaataaacattaaaaacacataaacatataaacattatagtcttttttttccctataagtctaccacaattgccattaaggggacattaatttgctttggtcataaatgcaataggttttttcactgtgagtgtggctaaccaccagaaagttaggatggcgacagctccagcaagaggctcacatgggatgcatccatgaagatataaccccctaaagaggtgggagtgtgtgcacatctccaagtaagaagtgatatggagaaaatacagtttgttgcatttctgcaaatcaattaaatcatcaaaaaaccttctgccatgttttatccaatatggctgtttagtatacaagagttgaatctacagtaaagctcatttgggctagtgtagacacatctgtgatttgagacactcagagatattcattcattctacagttttagagtgcctgctatgtatccagcagtgtgcttgctgctgagggtacagagattacaaagacagtctctatcttcatggtagtaggggaagatttatcttaaattttttttttttttttagagacatgatctagttgcccaggctggtcttgaactcctgggctcaagtgatcctcccgcctccgcctcccagatagctgggactaca

SEQ ID NO: 93 - DNA, 2001 bp, Homo sapiens
tctctggcctctggcttccactggcttttcctttggaaaaagccagttcaaatggtgctcactctaggaatcgcttcaactcggagccctggaggtgagcagggccgggcacgggccgggcacatacaccccctctagggagcagggaagggcacaggccaggggaagctggagacaaaagaggagacagggacagagataaaaacagggactgtgacaagaacacaggatgggaagggcagagacagaaatagaggcaggcttaaggatctagacaggtcatgccagcccaggttcaaggcagaggggcgtagggggacagagagagagacacaggaagcacagaggggagaaagctctgcttagcagacttgccaaagctgaagaccccactgttgccacaaaggggcccacaggaaagctaggggttgcaatcgcctgtcaggacccacattttcaccctgagctgtgcacactttccaacggcaagcaaaaggagcacaatctagttaagttaggactggcaagattcacttctgatttaaaacttaagacagcaacaataaagatactcagctgaaatgccacccaacaaagccagctcccctggaaagttaaagtttagcacccccatcccagcaccctgtacctttaaggaagagaaggttccctccgcttcccagccgaagaggcaggttattctaggatataggggaggcttacctaaaactgagttattctcctttcctcctctggccagcctctcttcactctggggagcttttaccatttaaggtcaatgttacatctggctccggagctcagagccgagaagcctctggctgcagaacctcggccgcacgcctcctctgagaggggctgggacttagagccgagagctcctctgggagccccactccggcctggctcgggggataatggcagggaaagccgaccagctggcctcagggctcagggcaggctgggcctttggggcagtcgctcaccctgggcccctccgatgggtatccacacagcagatggcagtccccatggcaactcgtgccactggacactgtcctggccggacgggaacaaagggaccctgcagatctccccacccgcctggagcctccagcaagcatgtgagcctggagaagaccctgggccagcgccaagagaaactgggaatggttcctgtcctggggattcgggggcaggggctgctcccactgcgttaaactcttgggattgccagagagaatggcctgggagattcagctactcccagacttggcaccagccccagggatgtcaaccttgctcctccattaaacagcctgtggtcttaacatcgtggagcctcagtctcctcatctctaaagtggaaataataatgataatgcccgactcactaggtagtcaggggattaaatgagatgttagtcaagtgtttaacacactgccagcatctcataagagctagatcagtatgaatgagcttcctgcccctgcccaggaaatccataccctgtccatacacactcttcacctgccttgggtatagggcctacccaaggccaggatcatggagctccaagctgggggcaagccacatggggacttcagccccttctgtggttagagcagcacagagaaaccctaggtaaagttgctagataaattacaggacacccatttaaatttggatttcagataaacacaggacatacttgtattaaagactgttatttatctgaaattcaaatttaattagtcatcctctatttttgtttgataaatccgggaaccctagcttttggccatcattagccacccagaaaacattcagtagtcatggtagagagcactagggattggagctcccaggcctatggcctcaagctctcctggcaaacacagtccttatgctcagctcctggccacagatcagttgtgcagcagtggcagatgctctccactgagcctggcccactgagc

SEQ ID NO: 94 - DNA, 2001 bp, Homo sapiens
actacaaagaagaaaataaataccaacggaaatcacactgtatacttctaccatgtgggggggcacttattcagatatctctctttcattctctgtccacttaaagaaatatgcatacagtttttaaagcatggagtaatactctacctgctgtttctggagtagtattgttgtagacagagtgacctctttcaatggcagatgacttgattcttggtggtaaagcaagtggttggttataagtatggggtctcattgtgcctaggttaacatttcttttccactcatatgtgccattgagtaagtgatttaacccatctctctgtgcctcagtttccttatacacaaaagtggggacgataatattacctacctcagagagttggtgtgaaggttatatcagatcatatatttaagtacttacttaagacagtacctggaacatagtaagtacttactacatttcaactattgttcttaactaaataagttttaacttcatagtaacaataaggggtttttaaatagaaaagtggaatctttttaaaaatacttttcaccatatttcaaagccattcttttaacataaattattactctttttgcacacagctatatacgttatccactgaaattagtatttgcaggtaactacccttttgcggaggcctacttaaaaactatcctgttgaacacaaggtacctttaagtgagacagttttacatgggcttcctttataacacattagtccaactaaaaatccttgttaattattaaacccttaggtattacaggaatacctgacactgcagattgaaaacagacagtgtttgtctctcaagttaaaccaacaagccgatagaaaaaggtagttatcaagagatttttaaaacttcaaccctttttctcttatagttagtgaagagagtagaatatctccagttttggctgacatctctacaacctgaacaattggcttaaacttcacttgggattcccggttgcttgttttagcatggcaaaatttggcgttcacagaatccttcttctggctatttctctgacaaagtgtctggagagtacaaaactgctggcagaccttaaaaaatgtggtgacttggaatgtgaaggtaagtttgcttcccccgcttcttctcctcctaaattgaggctctgcaatgacataaaatcttattgtggtcacgcatgataaaaatgagtattgataccattgagaaatatttggattatgaagtacagcttggtataaaaaagaaatgttggaagcaaagcattttgtggtagtatttaatacaaatatacaaatagcaaaaatcagtctgctatagaaatatatcagaagttaaaaaactagtcagtccttttactatttttaaagattaatctaggagtctctgagcctattctggctcagaaggctgcccattaaaaaaaaaagataaatctaattgattcatataaaaaaagacatcaggctaatttgataggcctttatttatgaaggccttttactttttagtttctgtttcacactcagaactttctgattctcatgacgttcctaaaaagtgtttttatttcaaaccatcatgatttattcttctaaactttactaaatgttaatcatttctgtgttatttattatgtaaaaatactgaaatcaggactagaatataaattttttatttatatcattgaatataaagctttctttactgtaaatggaaattaatttccattctattgttaaagaacttatttctgttattagttttatggtgcaaaataaattacatcccaatatattagagttgctttaaataagttatttttccttttgtttttattttataataaatacatattttttctctctcagcagaatcaaaaattgctttaaataaattctacctttttggttttttttttttctgagatggagtcttgctctgtcacccaggctggagtgcaatggcacg

SEQ ID NO: 95 - DNA, 2001 bp, Homo sapiens
agggctgtggatttaggagggggcaaccgataaactgtagaactgaagagtttggaatatttgcataggggagcacttgggctttggatttggggtctgagagtgagaatgcaccccaaagttgtggatttgggcattccaagggagaggaatccaaaggtctgggttttattagggtgagatggaagtgtggctgtgcatctggaggtcttctgttgcggaggatgcagggttaagggtctcaggaggggggactccaggcatatggacttgaaagtctcggggctaggaagacccagggctgtgggtgtggagtttctgggaggaggagtgtgtagcaagccagcaggtgctcccagaaagcccagcagcccagcaatgggcccaggggctgacttgattttgcttttgcccagagctatctcttgatgcccagaatcctcattggaacctcagagtgaggccaggatgggtgccatggaacctccactgcccatctctggttgggaaaggggtggaagaccagggccatatttcaggggaactttccccacacctggagaagagtacctggagttagcggttggcagagggattggctaggtctggagaaatagtaataacaacccccaaatggtggaaaaatctcaagtgtcccaagggttgtccgctggtttccaacccagatcctcagggaaacccactagattgattgtatcatccctattaatattatgattgttgtttatgtttctcattttatataaagatctacagaggacagaaaacagacagtgggcaggggacatgtgtgagtggccctgaactcccatctctcctgctctgtgtacttagttcccctttctggatttggttttcctgccagtggaaagggagtgtttgggaagagaagatcacatttgagggtgagttaagtctcctttaagggcgattcctgctttgtcaggggctactactattagcaggcagagggaacagtcgcagaagagattggggtaaactccagtgttaggcacagaggcagatcctggggtgggtgtgagtggagaagggatgagtagccctcctgtccttccttcagggaaattcttccttagtaaacaggacagttaccctttggtgagcatggtcatatatgggaagattaagtctaggtgtgtatgatctggttgaatctttgtaacagtgaggttggttcatcgtgatcatcctcattttgaagatgagcagatctagactccataaaccctcttgagtgaggtcacacagggagtaagtgagggagctgggatgtgaactttctcctcgccccccttcattgactggcctagatcaccctgggagtcaagtgcgggccagccttagcaggaggtgagtgctgagactggcatcagcagtggacacactgatgacattggcgggttctgatatcagcatcaggtatggccagggagggaaaggcatcgtgttggctttgggactgcccccaggtgtatgcgaggatgggaaacaagttggcactgaacttgacaccaggagaatattgaagtcatgtttggtgccaagagggctctgtgtgttgtctcaggtgtcattgagggcataagagtgtcctgcttcaggagggcataagggtggcaccatatagggtctgaggttggtcccagtgggctctgacatgagcatgaggcaggtgctgatattggtcctatgtgggtctgagatgagcctcagccagaccctgagaggggccccagatggccacaggacaggctctggtggttggaccatttagggttgcacctgtcctctgggtcaccaactttggacaagtggaggtgggtgaggttggcacccccaggactgacatcttgctcaaactgttctaggatgctgagctctgagacctgtgtggagggaacaaaggtcagagaacaaggcagagagaggaaagttgtttgttgattttggaggggaaga

SEQ ID NO: 96 - DNA, 2001 bp, Homo sapiens
ctttccttggtagctctcacttagggactctgaagagtcccatctcagaccccccaggatgtctcctgtaccctcatgccccgccagggccaagcctgctttgcacacctctgtagtcagtgcttctccgagggtacacaataggtgctcacactgctgaaagacgaaagcatgacagggcattctacagcagccctggtcttgggccaaaaccacagtggcatcctcagctccttgcttcctcccactccgcatccaattcaccagcagagctgatgggctctacctccagaacccaccagaatcttactggccacaccccctcctagccccgttctgctccaggcctccggtaccttgcacctgatggctgcagcggccacctctttgggcttcttccttccaccctgcctcctacagtctatgctgttgaaatcgaatcacgggtgtctgtctctgctcacaaccttgcagtggctcccatgacactcagaacaaaagctgaagtccttccagtggccccccccccagctgcatggcctgccctgctgtcacctctcagacctccccgcacccttgctccctctcctctgctccagctacacaggccttcgccccatggttctccaggcacacaccgctgcggatgcattccctcctcacggtcaaaatacatgcagctccctttgcagaacgttccctcaaatacctgcctggcccactctatcatttcccttgttctctgggttcaaatgtcacctcctctgagaagtactctctaatgatctgacctcaagcagccacccagggccgccacttgccctctttcttcttcattttgcttcaccatctgacatagtagattatctcttactgcctctttcccctactagtccagagggagacatactatactgtcgtatccccagcacccaggaaggcttggtccgttcagcccttggtcagggctgaatggatttctgttggatgaatgaagggacggcagttaaagagccaggttgccagctgacattgcccaatcttgctgtgtggcctcagacccacccctgcccctctttgtgcagctcagagacagccctctgtgctcagggaaggcctcttggatggacactccacctgctgattgtgacagaccccaaagccgccctctgccccctgcaaacctcccatgccccgtaccttcttcagccactgcgtgttgttctgcagtgcctgctccagctgtttcacctgctgggtgggcaacttccccaggtgcagtgggttggccagtgattctctctggagggtgttggagtccctggagacctcaggccccggagggcagggctcagacttgggcagcaagaaggtgtagctacagtggccgtgctggactacaagtgtctcgcagcccctatccgcctcctgccttgtctgttgagccacagacatggtggcaaccacaaggaggaggctgccctgcagcatggctagctgggagagcatctgaagatgtgtcaatggcgagggatgtctgctcagagccctaggggctgtgcctgggatgtcctgctgcagccaacagtggccaggcttgcctgcagctgcagctacaaacctctgtctggccgagctctgtccaggcagctatttatacttgctctgaataccacggctgctctgcttgcttcctctcaccgcctccctccggccccacccgacaccctctcacctctattcttcactccttcccttcctccttcctcagtcttggtggcacgtcgcccttaccctaagcgtgcaccaaccttgctctgtctgcctcacctctgagccctctgattatccagtgaggcctggcttccctgctggaggggagggaagaaaacagcacacagacacgcgcacacactcatgcacacactcacgagcacacactcacacgcacgctgacacgcacacacattcatgtacacacaaacgccaagctccaatgct

SEQ ID NO: 97 - DNA, 2001 bp, Homo sapiens
ttaatatgttcatgctattttcatttgtgaggtctgtaatccatcagaacaacctgacagtgattaagtaaaatcaaagagactttgtaaacatctgtgtgcttttattgttacaaatgcttcattataaaaagatcagtcaaaagcataaaagtaccctggtttaaactttttctgtgtttgaatgagaatgtttctcttggaacatgctcatatcaggcttccacttggcaaaatcacgaattggctgatgttgttttagaggctttctcatgtccccttccaaacatcactcttctccttctgtggacccctttctgttatctaggagcccctcatgctgccttccacattctgcctactgcacagtcatgttctggacccctcagaatccccctctaccattacaatatgtttttcctaagacttacttgcattgaaaggcttgccagggaagtgactaatcttatcatcttgtactaaatgttaaattaatcctcattctctctagaattgttttctaagcagggattcctagagatgctatcaatatctcagcctgaaattgcataggtattctggtgaatagcaagagaaaataacgtgagtataactggaccttttagaaaagtgaagtctattaggtcaccttagttggcctagggagttgatgtaggaatgctcctctccctgaaataatcttccaggagaggaatgtatccccaaatagcaagtgaatatccaatggcttctcattgcacttaaactctatattcctttttatgaccaaaaaggcctggcttctccctaactgtctcttccttacacattacacctcttccttttgtatgtgaatgtgccaagctaccacctcatttctaccttaaggcctgaagtgttatgttccagttcttggcatgcaaattactcttcatcacttgtttcccgacacaaatgccacctttccagacaatctaaagtaacctcaaaatcatttgctcgcacatcactttgttttatttctcctggtattaatcactatgtgaaattttctggttcatattatttgtcacctgcttcttccaggcctcctcttacttcattgtctatcatgttcattaccgtatccctagtgttttgcacagaatctggtacatattcagtgctcaataaatatgtgctaaatgaataaatgaataaattaacctaaacataagttttacacttaagtaattgagcaagaagtctttgttgtactaatattggaaaaggcaggggtgggggtggtgacgtgattataaccaatgtaaccagcttgcatttataagacttgtattgattatgaactatagcattttttcgttaaaaacattatcaatattggattgtaaaagattccattggcctttctgaaagactgtccttttgatgagatgtcacaaatgaatgctaatgtcccttgagaaatcctgccaacagatcttattcttctatcaatgttaagaaactgcatttctctttggccaatttctaggcttatggatagtctctcttgctaggacttcagaagccaatgagcaacttgcctgtcacaagatccgacgaatgatctctccctgcatgcggtgtagctaagcagagaagcagctcttgtaagagaataaccaacacagcatggaatattgctaaatctacagtgttttatccagcaaagttcaccccagtagcctgcccctgccaataaagcctagaaatttttcctaccaggcagtcccagtttagttggttgtagcttagaagcagacatctgagagcagttcagcaagcctgcagtcagatgcttttgtagagctctcctcttaaaaggcaaaattgaggcaagtcaatagattacaactttctgggtttgcttctgtcatctatcaaatatcacaattcatcctgcttcgctaaagtgaaactccaaggatttgtttttaaaggtgtccttctttctgtgaa

SEQ ID NO: 98 - DNA, 2001 bp, Homo sapiens
ttttaaattatctcttttttattacattttttggaagtatttctcaatttctctctattcacattttctgttcagcccttttccttattccgtatcttcattttcttggtagtcctcattttgaagtcattcttcagttaaataatgtcataaaaagatggagattaaacaatatgtttttcatgagagttgattaggatcaaattatttgaaatttctaacttagatgttttcaaaatcaaaaactaactaaactaccttgtattttcttaagtatggactacaactggtgaacaagactcttcaataagaccccctgtccccaccccactcccatttttcactgagcagcacatgccagatgccagggcaggagggtacgtggtcaccaacctgctccttccaggaattcaagatgtgccccagtctgtaggcatagtagtccttgctctgttggttgaatgtgtggacagcaaactccactgtggcagggaggtaacgagccatgacattgtgttcatcacagtccctttgctcgtggaaatgccaggcatagatcagcaggatctgggagcctaagagaagcagcagcagcgcccaggacagacctcccttccacggcaggcccagcatggtgctgactgtaggcaccgctgactttgctcttcccagcccccgtgcccacaggagttaattgagcacagacccagcccttaatcttatctgctcagacagccctgtgggacaaacttgtctcaccccacactggcaacaggcactcctacaggacaggaaggacaggatcctggattaaagggaggcagctctggatgtggagtcactcatctgccatccttctgccctatactctctaggccagcacaagccacaggcaggaggggagctaccctctgccactagctttgaaggctgctccttcagaacccacaggttctggttgtcctcaggtgcttggtgtccttggatgattctgtgccttcgcctcccacagcgaactgagataagacctggggacatctcgactctcactctcccctccttcaccccgtcgaagagaggatctccgttgttcagaaaaataactaagatatcagtttgttcttttgttgtataatacctcaacagttgtactctgtgaaccaagggaaaaaatgaaaaaaataaatggcaatttttatcaaccattttgacctgggatggacaatgagtattgtgagaaaagggcaggaaagacagagacccaagtcctgaggccccagcctggtcatgcccagcccagccctgcttgcatttctaaaccttgtcagccctggttccagctgagccagagccaaggtttctacagggcccaccgccctacatctgcagccccaccacacacactcatgttttattagactggaatatcattctagaccaaatttttaaacacttctctgggagatcacatgtttctgcctcagtgagatagaggttggaccattcttcagccccttgatcatttttgtctccatctgaccatccttcttggtgttctagagtgagctattttgccagcacttaggtgatactaggtggttacttttaaatcaatttcctggcaataactaacctgaaagaggaaataataatattttgttgctgtaagaatgaacattaaatagtgcaggacaggtgaagtatgcaaaagtgagagctttgagtttggctgagaagactatggtgctgcaatataggcaagaacctagcattgttgccagataacagaatagaaagttcagtcccaagtcagggagactgattctacaagaactgttctgactgtgtcccataccatgttcccagatcacagcagggccaggcctgggaagagcatcactgcatctgtccacactagcacagcactggccaggtcaagacagctgcaggtgaagagagtcatgctgcctgacccaacactggggtggtcccgagtgtctc

SEQ ID NO: 99 - DNA, 2001 bp, Homo sapiens
agtgataccctcctgccatcttaaacggctctctgccatctgcttcctcctcctaagtggttttccttctgtcctccctgtttaatgtttaagttgtgcacatcctgtgacatcctggagacccatgttcctgacctacttacctattcaaccatctcttctcacacatggggtagcaatgagaagcacagggggtctctgcaagggagtagggaggtggagagagcgggggcagccacacatcacactgctgttctcagtggcctcctgctgtcaacaagctaacattaataaaacaagcccactcttctgctaagagactgtgacagggtaacatttattctaggtagcttttcaagaaaacagctcttgctcccacggtcccctcggagggcctgcagagctccagcgatgtcagagggcatctgtttgtttctcctttgcttataaaagggacctggaaatcaaaggcaggctcttagcccttagcagtgaggatgacagctctgcaccagccacacgcaggcccctggctctaggaaccacctctcacccagacgcccaggcacagcatggtggggagagagtacaggacacagggcctaggcctgctgccccgcccacagcccgccccctagccctttctctgaggccaggtgtactcacctagcctccagggtcccctgctcaggtggccccagaggcccaaacccactgctcccactttctgagacctgggaccctcacctccccaactcccaccatagggcgacactgaaaagtgtccccttcccccccccgtttccaagtcaccagcttgaggatgcccaaggctcaaaggctcctccgagtggcaccccgcatcgggctgcagaggtcggagaggggaggggatggtgggtgcaaagggtggcttcttcaggaaagggaagcctgtccctggctcccagcacctgtcacccctggcgatctcaccccgcggccatcattcccgtcctcacggcccatttcagggacactgaggctcaaagagccacgtgatttattcaaagcagctcgagccaccacttggagccacaagaaagccctggtccgtgcctcctcgccttggtccctggcccttcactcttcgtccaggctctctgacctctttccctctgcgcccctgcctcttccccaggcctgggtccccaaggcactcggccacgcgctgggtccggggaagaggcgcggccgcggccgaggcggggacagggaggcggcagcccctcctgcgcagcgacggcgcgggcgggcggaggccggggcgccccagccccccgcccccgctccctccggccgtgacgtcagagccggacggggtaaactgagcggcggcggcggggcgctggggcggagactgcgacccggagccgcccggactgacggagcccactgcggtgcgggcgttggcgcgggcacggaggacccgggcaggcatcgcaagcgaccccgaggtaacacggccccggttccctgctcgcagggcgcggtgccggaggggagcgggcccgggggaggcagggaggcagggaggcccccagagctacgcgggcggggctgcggggaccccggggcagccggggcgcagggcgcagggcgcaaggtgcacggggccgagcgtgcagtttgggaggtggcacagttgggggcggcggggatcagggcagcgtcctgggggttctaggttgggtctccagattgggtccccgacggcgctcgcgggcgctctttccagggagcgcgggctctcgtggggacgcgctcgaggcaggtgagccgaggctggggcgcccgggggtgtccccggatgccggtgcgcgcggctccctgacccgcccttccgcggcctgggggacgggagaatggggggtccacggccgccttgggtcctcaccgcgcaccgcgcccgcagcggagccccggagccatggccctgagcgagctggcgctggtccgctg

SEQ ID NO: 100 - DNA, 2001 bp, Homo sapiens
ccttccaaaaatgtgtccacctgggcccctgccctgggactcatggatttggagttgtggccacacggttgaggggtgcagtgtccagtggaatggggcaattgcgggcctgggggcccttggcctgtccgtggcgggagcatctgcaaggaggagccccagagtccagggagcactgtggggagctccttagagctgaactcacccggcgtcaactcatcaaccctccacccatggacaggggtgcccccagcacaggagaggactcagccctctgcccccacgcacggtgggtgcctgtcaccctgtcctgcccagcggcccgagggcagcagtgggtgtgagggcagcccccggcctcccaagagcagctgagaggatccctgcgggaatccgggcttcgggtgcatgcgatctgatctgagttgtttctgacagtgacagagtgacaatctataagtatctcaagatcaaatggttaaataaaacataagaaatttaaaacgattaaaatatgcctcagtcttgggaatcctggtgtctgcaacaaaaaaacttgaggctgtccttgcaaacggtcctcatggtgctgggggtggtgccaggcccggcgtttgcctggaacacagagcatgcgagaaggagcagaggacgtgggtgccagggaaggaccctggcgtcagagccagggcctggacctgagttacccattaacggcactccactggttttcctttaaagaaatgaataaatacaactgcagtggggctgcttgtggtttccaaacgtcggcagaggctggagacggctctctagtgctgggtgtggagtgaggcaccacccttgccctgaagcctggggcactcagtcaccatggctcatgccccagaaccggacccggccgcctgcgacctcggggatgagaggcccaagtgggacaacaaggcccagtacctcctgagctgcactgggtttgccgtgggactggggaacatttggcggttcccatacctgtgccagacctatggaggaggtaagcacccacctgcgtcctggggcagacccctaagcaggggaggccgtctccaccctgacctgccaggcgcctgccacgcatccccagtgcttgggcagagtcactcctcctggaaaccagggtgcaccattgcccacggcagagtcagtcccctcagggcccacctgcagtcttctgggacctcagggcagcttcaccactgcccacagcacggaggatccagtcccctcggggtccactatggtcttccgggacctcagggcagcttcctgccaggcgtcaggttgaatgggccgccctgcccgacacacacaccctacaaacagtgagcgctcttccatctgaatgtcgtcagttctttaaagaacacacaatccgctctgggtttcctcaccacagccttggtgtgtccagacatgtgctcaggtgcccttctccgaggttctttgcaggaatttgggggcccacccctcgcctccctggctttggttctctgggagagccctgtggggggtgccaggatgttcagaactgtctgggtgactttagtgaccttggactcaccacccccagaagtgcattggtgactgaaataggctcccacgcacctcgagcctcccctcaccctgtgcctgcttctcttcatgcaggggtcacactctgctctccatgtggcgacacaggatgtagggacaggcatgaatcactgctctcagggagccgacgtacaacatcacgcatggacacccaggtcttccccaggcaacacgggcaggggcttcccaggccgcagggtcctcctcaggccaggccgggctgcttggtcagcctgttccccgggccctcagctccttccctgagagccccgcacaactatcccttgttataaggttactcctttaaagtgtttttcctcctgcttaagaaaagattctcaacaggcagaaaatgcaggaaatgcgcac

SEQ ID NO: 101 - DNA, 2001 bp, Homo sapiens
tctccagcttgggggtctcctactggattcctctacccagggctatgtaggactccagcccctccttctctccatccccagctcctgtcttctcccactcccctctccaggcacctgaggttgcaggaacccttcagacccctggggacttgggtaccactctacagttttcgagtggggagaggcccagccagccccttctcccattcatcaatgaagatgcagatattcagaatggccacatagatagagtgacctattaaggtcgcctagcagcaggtggctgggtctgactgaaaatcaggtctgaggacccgcaaagcgtgccttccactggacaggctggtctgatgccattttaagtcacagagaatccagtgcaaatgtgagacagttaggaggcagctgaattaggaagagtgactgagaatggaagagagagggatgcgggagagggggtaagacagaaaggggaggagagcaaaggggaaggaaagcaggtggaaaggattgagaaaagagagatgcaggataggcagagacagggagagagggctgaggaagagggagagagaagaaaaggatggggagggagatgaggaatgcctgaaagggttagagatgaaggagcaggatggagaaggatggcccagagatctgggacatgggcgaggggctggaagccagctctggacaaggcgtgtagagccactggggagcccactgccttcggagctgtgcctcagctctccagcctctcctcgccaagctcagctcacctggctccccctgcgcaccttcacccctcccggggccacaaagacacacagacacacacagacacacacagacacacacacacacacacacagacacacacacacacacacacacacacacactcttaccgccccagttgcaaagtctctcccaaagtggttcagcagctgcttatggctctctccttgtgtccccacaatggtcagtgagatggagtcccgtgttcccgacaagaggtcggtgcctgtggccaccctgactttgtaggtggccatggctgctcttcaggaggcaagaggggcactcagtcccagacaccgtggaggggccacacgaaggcccaaggcaggcaagagaagccaggcacagagtggagtggacagggctggcctccgaggtgcagtggtgaggtggcgaggtggggtgactaggcctgccagccaaattctggaaaagctgctgcccttggtggccggggtgggtgccgggcaggcccaggcggagcccagctggtggtgagtgagcaggtgtctgggctccaagccttctgggagctggtggggaggaagagcaggcagggcactgggcctgcctctgaaagggcggatagaagatgccgtggatgaaggcgccttcagggtgggggagccagtccagcctgagccagggccctagggaagttgcgggagggggcagggtgtggtccccagatctttatcagactaataagctccttcctcctgccagtccccattggctacagtgccaggcacccctactaagcagggccatgggcagtgagtgacttgcattggttctgaggccactggtgggcgggggagggtagggggaggggcactaagtggcagcgcagctggccagcctccaggccattcctctctgggaaatgggacaggtagtggccacagtgagaaagctggcctgtccttctgccccagggcccagagtctgtgactggaagggtgggaggtgttgaagctagggctcggggaccacatgggtgcccaaaggcctgaagatgctgcggggaggaacgatcagggtaggagaaggggatctgttgcttcctgtggggccaggatccactggtgagggattggagttggatataaaatcagaagcccaaaaaaaaaaaaaaaaaaactaaattagaagacagagtgaaatctggggcataagaattaagttaaatagctgggc

SEQ ID NO: 102 - DNA, 2001 bp, Homo sapiens
agcccctccccgagcctggggcttccccaccctaccctccactcccagccttgaccaggcgaatgcagcctccccctcctcccagcgcgcgcgcgcgcgcgcgcgcgcgcacacacacacacacacacacacacacacacacacactctctctctctcacacaccctctcccccgacgcctgggttcaggcttggccccactgaagaaggaggtggccagagttcctctcacctgatctggggtcgccaaggctgcagggagggtgtagtaaccttcctagggagccagaatggggcccagatggccacatctttcatcactcctctttgcgtcagtttctccaccaagatcagggtggctggaaggtgcctttgacaagatgaggatggagccagacagtctttgggcttctcggtgtgggacagtcacatgtggcacccacagcctgagcgcacccggggtactcagagcgggcaggagaagcacacgtctgggcagcttggttcccaccctcctccaagccctgcgcctgcccctttggaggggacagctttggcctcagagtttggccctagcagaggttcaagtagcaccaaagattgtcagcctgccccgatgcttccttgcctggcagggatgcttgccaacagaggtatcctacccttctcgggattcccccggtctgcactttgggtggcctggattcctgctccaagctgtagacttgcttcttatgggatgaggagggccttgggatccaaaattgagccaagcccaagggcagccgggaaagagcctggtcttggggtgcacaggcaaaggcaaaccgccttagggagacccagtggcagcgaacgggttaggcgccaccgtgctcccgcctgactcaccctcgaagctctaaccactgtgggctatttttgccagagaggggccccaggctttccagccgccccccgcccctgccagaccgcagcccaaggcacggctggccccacgcgccccaggcacattgactcagactgcccccaagcaagcagaggaacagggatgatacccacaaccacagtcacctcttccttggtggtggcgttgcagctctgctgggcagccagcctccagtctgtgctgctggaggtcctgcccgtgctgcaggggtcactgttggatagcgggcacagcagtctgagacaggagatcgagcaaacttacatctgttgggatatttacagatcacctttacgtgccaggctgtgagcagtctggggaggtgaaagtgccatggtccctgacactggacagcctggaccagactgcttttctctgattccctacctctgggatggggaagtgggagcactagagttcctgaccctccttcccatttagaggacaagagccaaaccatgtgtggttttggaggtcaccagaatctgcaagccccatctcatagttttcatccttacacctggattcatgatccccaccagtgtccttatcctttttcttgcagtcaccctgggattcctgccaggtccttggaagaggggaatgaggttgcctaagcctctcttctccatggggacctcatggacatttgtatttctccatgtgtctctgggtcccccttaggtttgggattattttttgccttctgattctacccttgtcaggctccagcatacagccaggtgccaagaaggggcgaggggcagaactactacaataggtttcaacttcctggtgaccctaggtctccccaagcaggactgttccaaatccaaggacccagagtcctgctccttctctctactgaagcccaactctgggacaagaggccacaggccggcagactgggtggcctgagctggcccgcagtgccctcagcccgaccggctggcctgagcactgacccgcaggcggctagagggcggtgggacaggcggggcctggagagggctgtgggtgcacacccattgcctaataccccaagcacgcgg

SEQ ID NO: 103 - DNA, 2001 bp, Homo sapiens
cacaagttgccctactgctgccgttgatgttctcatgccccaccagcttcaccttgggtctggttcccgcaagggtgagttggagatccttggtcctgagtgagaagtgctttgtcagaggcagcagagggcgccatgggagcacacaggaagggcgcctggggctgtgcagggactgcagaacaggctgcactggcacacagggttggctgctggctgctggctgctggctgctggctgggtttcctctattagcttaaacaatggaaggtggacttggataggaaatctacctcatgtattcctgagctgtgtctgcaccgttcattcattcgtaggcgactcatgtattcaagcactcatttatttattagatgcttgctgtgtgcttagcactgtgccaagccctgtccttagctaccaagagtttaaaacgtaatttggaagacaggatataagattatgtctcccctttgtattgcgactcactcatcaagttctcccagcatctagcattcatatctggcactcagtaggtgctcaacaaatatttgttgcataaatgaatgaaaagattattagtaataataccttttatttactgagctcttcccaactttccatgtattgtcgcaatcctcaaggtaagtcctgatattatcctattttattgataagaaaagtaaggcacagagaagttaagtaaactgcctaaggtcacacagacagtctgaagtcttaaccactactccaggtgcatctcagcagtgtgaggcaggccatgcttagggccaatgcttagcacacacagagattgcctcaggctttcagaggacaaagcagtcaggaggggctggggcaataaaggaaggcttcatggagacagaacaaaataaccagtgcaggtagaagaagagaaccatctttcctgatagagagagggggcagcaagcaagagggagtcccagggtggatcatccatactggcttccaggggtgacgggaaaggatgccagcaaggacttcagcaaggatcaatcagcaaatccgagggtgtctctcttgccaagccccgggctgtgtgcataggagaaggagaacatgaatgagaacctacctttgaggatctacagccaattgttggacagaggcaaaatatgtatataattttactccaggaaagcaagtgttgagagccagaagtggtatgagaatcatagtcaggaagacagaaactgtttccaaatgggaagcacagagcagtctccttggaagagagtgcattagaactgagccgtgggggcgggcaggatatcactgtggagtaggggaagggcactcctggggtggcaaggtgggaggtgggccctgtgttcccacagtgggcagggaggtagtgaaagggaagctggccggacaggcagggccattccaagagggctttgtgcgcagggctaagccaagctttctccataggcaatggggagcaactggaggttcgtagcaggagaaggacacatcttgcccaccaggaggctaagtaaaaacagttgtctcccaagttataagttcctggaacccttgctgggagcaggatttagaaaaatgatgctgagagatgctagaaacatattcgccctgaggctctctcactcagactgcaagaggaaggtatcatcagaattgcccttaaccaggaaccagaatagctgggtccccttcctgccaagtcagcaaccagctatgtgaccttgctcaggtccatctccgggtgtcagtttcttcatctacaatgcaagagggttgcccacctctgagaacccttctaaccccaaatctcaccctatgaatctaagaacaccaacccctcgccatcctaagtatcacagagccaggcaagcatgggtgagagctcagaccatccttgttggactaaaaggaaggggcagactgcccatggggggcagccgagagggtcaggcccccataggtcctcagcct

SEQ ID NO: 104 - DNA, 1925 bp, Homo sapiens
ctctttcaagaaatatgatagtggggggaggggaaacgggaagatggtggaaggaagggctttctggggagatggaggttgggagcgacttgagcagttgtgagccagaatgaaaggagaagcggaaggcaggagtttgaaggcttcaaagcaaggagcctgtccctaagagagctccagagggagctccgagggacagatggagagtagggttggtgggggctcaatgtggctggacttttccaggcctgtgcaatggaagaggtggaagtgttcatctctgtggcccccagcagcttaatagtctttccttcttccccttttcctcttcctaccttcctcccttcctctctagccacaaacatttattgagcacatactatgcaccaagtcttgtgctaggttatgttgctattataatgaacaggacagtgtatatcatccttacaactaagtgaggagacaaatattatacaagaagatggacaaatgtgtgtaggtctacatattgtataatactaacaatgggtaatgttcattgggcactttcaaggtatcaagacctttataagcatttcttaataaccttttacctaaaaatccatcccctacattaaaacttacaagatactactgatattaccttttgcagatcaggaaactgaggcacagagggatcagatgactggccaaaatcacacaacttagattcacctgtttccagacgcaagcacttaactgccacacggtcctgactatgttgagagctgcggacaccagtatgaacaagacctgcagagtgtctagggctgggaagaagccacgaattcatttactgccttttcctcaaggtgtggtccctggaccagacatatcagcgtccctggggaggtagtcagacacgcaggctcttcagcctactgcgacctactagaccgtgatgtgtagtttaaacaagacccgcaggtgtctggtgtgtagttcaagttggagaagtctggtctagagcccattcctcttattagaaacaggaaaccgaggcccaagcatttgaaggttatggacggtggtagaactttggggtttggtgagtggagccggcctaggccggaatgggcgcttccactcttggcccagcaggccagtgggcagggccgcgggaaggacgggcgctgcaggggctccgcgcgctcaaggtgctcgcgggctcccgggcccagccgggctgaggtggcgtgggcgccctgcgggccgggcctccgcggcgcgtcccagtcccagtctccgcgctggtctggcccggtccctcagctccaccccctgcctccccctcccgcccgctcgcgcctcctgaaaagccagctcgcccggggcggcggcgcagagccgggccggcgcacgaggcagccagcgcggccatgacggcggagaaggcgctgcccctgggcaatgggaaggctgctgaggaggcgcgggagtccgaggcgccgggtggcggctgcagcagcgggggcgcggcgcccgcgcgccacccgcgcgtcaagcgcgacaaggcggtccacgagcgcggccactggaacaacaaggtggagttcgtgctgagcgtggccggggagatcattgggctgggcaacgtgtggcgcttcccctacctgtgctacaagaacggaggaggtgaggtgatagtgaggagaaggggagggggcgccaaccgcccggtgggggcggggagccaggggcgagcgcgagaccccctcccgcgcctgcgtggagcggaacccgagcggagaacctcgactccaggcacctcgcgtgtgagctcgccccggagcgcggcccacctgtgccagtgcgcacgcgcacgtgccaattcgcacctgagggttccacctgccagcgcggggacttgcccgcgttctgtcccc

SEQ ID NO: 105 - DNA, 2001 bp, Homo sapiens
tttcagccagttttgcctcttgcactttgtccctgcctgtccccgtccccgtctccctctctgctccggccgccttggcgcagccctaactcgggcatccgaagctcctagtgggcgcagaagccagcggctgctgcctgcccttcgcctttcggaggctcacgggaacttggttcctttgctggggccttgaggaccggcctgggaaggaggggtggataagagcagtaagcgacaccggggcctggctgggcaagggaggaccctttggctacaggcgggagggaggccacccccaccccctaggtcttggccccacaggcttggcgcgtttctgggctggagttctgaatacctggtggaggtccacctgagccaaagggcaaggtggcagagatgcacccagggaggcctggtcctgcaccccctacacacacacccttccgtgaaaggacaggtagcagggcatagggccagaggggatattcagcatcccctgctcctttgattccacagcagaaatatccatgtaccatagccagagccctcaatctagcagggacccaggccaggcctctggaagcctcccctctctcactcatggttctacctcccactgtgcccattatggcctgatggggactgtcctagctagatgcaggcccacctaggtgtgaaaacccctgttctttgtggtccctgaggagcctgcctggcccttcctgcaggcccgcctagccacagctgcagggggcttggtgcctaccccatctctcataccaccagcacctgtgaccagagatgtccccaggccaccgcagcctcctccacctccaccgcatccccacagggagattcctgggaccccttcagaaccctgctggggcttattcgtgcccctggaggggacctgggaggcttgagggctcttctctgaggtgcagaaaggggggcacttgggggtcatcaagtggctttggcaggaccggaacctcagctccgcaccccttgagaggatctttgttcctccttgggcctgcagcctgcttgggcagcgccacccagacctggaccttcctgggctgcagctcacagcttcgagatgcaacccggctgcctctgggtcctggggtcccccagatgagcctgcagatctggtgccaaatagacatcgttggccagggtgcagggtgcagggtgcaggggacctagaacccatccctcacttgctgtgttgtctttccatctctaggtccctgactccccatttgcaaaatgacatgggaggtgggctttcctccctcagcctggggccaaccgaacacaccctctggggaagtgagacccaggcctggagacccacctgtgccagctgacctgagcagagggactctgatgtcttcccccggcttttgataggatgaggctcctttcttcccaggactcccacagcaagctcaatttagggcttcccaaggacccctccctgtctctcctatttgctgtgagttcagcaaaggacgctgcaggcagctgatatatctcccagaactgcaggcccacccacacaggataggtgcttgtggtgactggggactgacagccagcagggcttttgggagctgggaattcccagcagaactgatctggaatctggcttccactttgctccgggtgctccctgagcacactctcaacctccgcaggccctggcctcctcagctgtgcaatggaggcaacaaaagcacaggtaaggaagaattgcggatgaaggatgcaccaggaagcactcacttatctgcatctgacccctttccgtgcccctggcccttgacctggcttcccacctgcttcctttctgctcctgtgttcccgccatgctgaccgcgtcatctctccccttcccactggatgacccccggccccagatcttcctgccaatgacttatcccattcaggtttgcctgaatggagaagccactccattggagtagccactcg

SEQ ID NO: 106 - DNA, 2001 bp, Homo sapiens
cagggtgaaggagatggggctggagaggctgagcagtccgggttcgctgtccgccacttcggcgcggaatcagagcaggacttgctgagccctcctaccgctccctttccccctccccctctgtcttcccttctgtttccttttccctccccctggagctgtagcggcagcagcagcaggaagccgagccgggttgagcgactcggaggcgagcggaggagctggaatatggggagtcagcgaggacggtggggccaggagcccttgggagggcctacggagggagcggccccaggcgctttctagagcgtgagcggtgggggagcaggcgcagggtggcacgagcggaggcggggcccgggcgtggggcacggctggggaagctgccgcctccggccctgcccggctgcctccgccgcggccagtggctatggagcaggcgggcaccagacctgcggcgacagagcatccacggctccggcggcccatgccctggctgctgctactgcctctcctgctgctgttgctgctgctgctacctggccctgcggcctcccagctgcgatactctgtgccagaggagcaggcacccggcgcgctcgtgggcaacgtggctcgcgcgctggggcttgagctgcggcgcttggggccgggttgcttgcgcatcaaccatctgggtgcgcccagtccgcgctacctggagctggacctgacgagtggagcgctcttcgtcaacgagcgcattgatcgggaggcgctgtgtgagcagcggcctcgctgcctgctcagcttggaagtgctggcgcacaaccccgtggcggtgagcgccgttgaggtggaaatattggacatcaacgacaactcaccgcgtttcccgcggcccaactaccagcttcaggtaagcgaatcggtggcgcctggagcgcgctttcacatagagagtgcgcaggaccccgacgtgggcgccaactcagtacagacctacgagctcagccccagcgagcacttcgagctggaccttaagcccctgcaggagaacagtaaagtgcttgagctggtgctgcgtaagggcctagaccgggagcaggcagccttgcaccacctggttctcacagccgtggatgggggcatcccagcccgctcgggtacggcacagatctctgtgcgtgtcctggacactaacgacaactctcctgcctttgaccagtccacttatcgcgtccagctacgggaggactcacccccaggcacattggtggtgaagctgaatgcctcagacccggatgagggctccaatggtgagctcaggtactccttgagcagctacacgtcggaccgggagaggcagctcttcagcatagatgccagtaccggggaagtgcgagtaattggggggctggattatgaggaagcctcctcctaccagatctatgtgcaggcgactgaccggggtccagtgcccatggcaggtcactgcaaggtgctggtggacatcgtggacgtgaatgacaatgccccagaggtggtgctcacggacctgtatagcccagtgcctgagaatgctacacccaacaccattgtggccgttctcagtgtcaatgaccaagactcaggccccaaccggaaagtgagcctgggtctggaggccacactgcctttccgactgaatggctttggaaactcctatacactggtggtgagcggcccactggaccgagagcgggtggctgtctacaacatcacggtgacagccacagatgggggaataccgcagctcacatccctgcgtacactgaaggttgagatctctgacatcaatgacaatccaccaagcttcctggaggactcctattccatctacatacaggagaacaatttgccaggtgtgttgctctgtactgtgcaagccacagacccagatgaaaaggagaatgcagaggtgacctactcccttctggagagggagattcaagggctgccagtcacctcctatgtctccat

SEQ ID NO: 107 - DNA, 2001 bp, Homo sapiens
cccgatgcctttcatctgctgtgcctgtgctgagaccacaagacaacatactgaaaccaggaagagctgccccaaattctcagacactgctctgaggaaaaaactgtcccttagagtgaggaaaaaaggaggaaaaaggggctggtcggccaggcctggcaccctgaagtggggcgggcagagtccgttgggggaagagcacctcaggggaccaaccaaggcactgaccttgaagaggcgaaggatgttggccaccatgatggagaccgagctccctgaagcaccgatgacacccaccacacgttcaggcttggtgatgatgggtgggccgccactgccacagcggacctctgtgccatccttctcgatgagcgcctgcacaaaggtcagcgactgctcgagggcatgggtgtccctggagcaggtgtccagaatgcgggcgcccagcgtgatgttaggcagcaggtccgggtcgttgttgatgcgatccagggcgaacagcatggcctccagccggtggatgcccttttccttcttaagttctccacagggcttgccctctgagccccggccatgcaccgggaacaggcctcccagtgtgatgtccccatctatgcggatggaattcatgtgagggtggcctttgggctttcccagggaggaaggcatccaggggccgtaaaggctgaggagcaggcaaaggggcagccgggcccaccaccagcccaagcctctcttcccaggcatctcggaaatccctagagacccatgaacggcaggcccactcctagccctggcaggcccctggccccacggcctgggtgggcatgggcagggcagcttcagcagcagggggactgagggcagccaaccgcgtagcccatgctgctaccctctcccacctccttgtcactcgggccaagcacagttgcccgcacagtccaggcccacagacagcaggcagtggccggggttgcaggaaggctctgatccttgtcccgtcctgcaggcctgttctcggtggatgactgtggaaagggcagaatgctcctagcttggcgtatcttggcatcaaggagaaaacagctccaatcctctcctcctaccaaccttagaaggggaagagagagagagaatatcaaacagaagcccaaagaagacattagctagtctcatctctcatcaggagcctgagagaaggagatgctagaggttatcgccgaagattccataaatctcctgaacttggtttcctctccgccattgctcaaatctggcttaaatgggagagtgtatggcagaaattaggtcatgtttgaagcttcctaaggcctgtacccactgctatcagtagcccgggcatcagagagcacaatccccaggaacagcctcgaactggcttggaaggaagaggggccttcctcaacacatccacatgaaggagagcaagaaacaggtcctatgggcccaatcccattacagagatgaggaaactgaggcccagagcaggagagcattttgcctgagtcacgcagggagtgtgcagtttaattacaagctcagataaagcttggacaatgcctactgcagcccagctttcctgctgtcagctctggaacccgagacactttggctcttccgggactcagagacccaggaacacaaataaggccccctcaagaaatcatcctggaacgcagtggtgtaggctgggagctgctcactctgaaggccaagggaaggccagggaggagctgaggctgtgggggttctggattttcatcctgaatcagggagcgcaaggtggaccctgtactctcctgcttccaccccagctacacagctaccccaggcagagcaggggaaacccagcagcccctgcatgaggagcaggcctccattcagcttgcacatcctccctccctccctccctccctgactttcttcatctctccatctctccactgctctttctccccacccccacttcctccctctgag

SEQ ID NO: 108 - DNA, 2001 bp, Homo sapiens
atttgtattggaatagctttagatatacttcaatgtaaaaaatattcaagattcaactctgatctgtcctgtgtgtagttggttccaaaaggtagtgttttagtcagtttttagtcattaactatttatagacacactattcaggagcctaaacatttcaaaagaaatgtaaattattaagtccattccatggatatttgcaaatatgtggatgtttagaaataataatggaatattttcaccttgcaatccaagttgtgtggacttacatgagtatttaccttggtctattaatatttgcaacatgaacctatctttcccacataactctgaatgggcttagtttatgaagttttatacaaatttgctacaaactaacagctttagttatacattctacagatacatattaaccatttgctctctgcaaggcactatttactgccttattttacatacttctttctctttagcccctcttaagagatttttggccaataagggaataatatatccccagtgcttttttttttttttttttgggacggagtttcactcttgtagtccaggctggagtgcagtggtgcaatctcggcttattgcaacctccgcctcccaggttcaaatgattctcctgcctcagcctcctgagtagctgggactacaggcatgttccaccacacctagctaactttttttttttttttaatttttttagtagagacagtgtttcaccatgttggccaggctgacctcaaactcctgacctcaagtaatccgcccactctggccttgcaaagtgctgggagtacaggtgtgagccaccgcacccggcctcaatgcattttaaaagcatggtatgctgttgctaaaaccaatattaaagggtaaccatagtgataaagtatacaagtatcagtcatgattttatagtttaaattcttccacatgctaaccaaaattaaaaccaattcccaaaggcaatgagtacttacatgacgattgcccattttcctttttctgagatggtttttctgtatttcatcaagagatcagtcctcctacaggcaaagtttacaacagttttccccccggacataatatctcttgtgtttaataagtaacttttggcactgaatttgaatacattgttggggatgggtagggagtggctctagctacagataaacttagactgcccaggtttccttggtacccatgtttttaaaataatatacttcttaataaataaagtttaggcttttaaagttgaatagcttcttcccacctaaaagactaaacctgcctcacttctgaattgttgagttattctttcatattctggtgttcatttaaaatattatgccttttagtaatgaaagcattaaccaaatagaaaaatattactattttttactaacattgttattattattacagagaatttttcgaaggctacattgcattacactagatcatttaaaaaattttactgagttttgaatttaaaaagtaatatgtgcagatggtaaatttgtttctaataatacataaaggtcctgtgaaaagtcggcctactgacttcctttttccttcccccagaggttttccgtgagtccttcctgaagtggtccatgcatattcagctatgtccgtcggtctgcctgtctagttacctacctctctgtttatcttgatccaacttgtcttttcttcctcaccaaatgggtttgtacaatgtattctgttccatgccttgcatatttcttgtctttcctttttttttctgagattattacaacatgtttctttttttatttgtacaaacttatgtggtacataagacattttgttacatgcatataatgagtagtgatcaagtcagggtattgagagtgtttgcctgagtacaatagatttctgttgagtacagccaccgtactctactatccaacactgaatttattccttccatcttagtgtgtgtttgtaccctttaac

SEQ ID NO: 109 - DNA, 2001 bp, Homo sapiens
agctttctgcccctccggggaggcggatcccagcccccaacccaccaccctcatccgcccagcgccttccctgtcctggtcccgaactgacataaacagtaggttgccggacacggtgaagtccttcagaaccgcccctggtcgggggagaggtgcggcatcccgggacaggatcctgcggaccagctccgcttccatgggcgacgagaaaggcatcgtgatgtgcctggtggggacccagttaaggtgccatctcctaggatttggccggtgcccatctcatcccctcaggctgtctccccacgcactggcccaggtcctgcagcaaccccacccagctgtcttgattccccactttcctctgttgcacctcaagctgactcctgcgtgcccttccaaggccctccccaccatgccccctgccgcatcagccgggacctctccccatcgcttgacaccacccccatccagcccacaccccagccaccggctcccaccttgcacgcttccccccaactcctgatcccttcacacacccaagaacccctgtctcctgtccccctgcccctcaccccatcgcccagcacccaactgtgcacacgctccacctcccatcccctacctactcccttcctctgagccacccctcgacctccaactcccagcccccacccccaccccacccggccccttacccccatctcccaaaccccaccttccgcacctggccctgccctcacccacctcctggccctgccccccacctcttgaccctgttgtctccgtctcacggcccccacctcgccaccagaacctcctagaacagaacagaacaggatacaactgaagcaggcggctgtccggcctcctggccccgcaggggcaccttccatcctgcgcagaagcggcaccgccatgcggaccccgcggggcgcctcctctcggccccgaggcccttgctgcccctgcgccccggggacctctgccgcccgtggcacccgcctctcctgggccgccagcattgccccctgggccatcaggaatgccagggcctcctgggccatcagcatcgcccgtcgaaccccctgtgccctggccttcggcctgcatggctccggagcctctgcccggctctcagagagaaggtcagggcccacgaggatgcggaggcagagaggctgcaggaagttccgccccctggcgtgagatgggcagcccgggatcctcagggcgcctgcgcacaggggccctacttccggccctgggagaccccgagtgagccccggagcacgtgaccggttctcaccaaccccgcccctccccaagagagcccgggccggaaggtggccgcaatgccagcttggacccctcacccgtgagcagccggctgtccgccggacccctgtcccgggagccctgcagggagtcaggcactgcggggcccagcctgtcccatcccccgggtctccctcacatcgaggagcaagacgggcctgggaacacggggccgggactgtgcggccatcgtcccggaccctgcctgccctgtccgtccttgggggagcgcccaggacagaccccggggggcaggcctctaactgggctcagcagcctccgtccctgtcctggtcgcccagctggtggggtagctggaactgcatgtctggtgggcgtggcctcgcgccagccaaggtctcccaggcgccaggtccagccaaggtggacgggagggcccagcgggacccggggccggatctcactcctgggttcaacgggttctgtccctgtcctagtcgccccgcctggtgggggagctggagctgcgtgtggggtgggttgcgggtggcctcccaccagccaggggctcccgggctctaggcctggcccaggtggacaggcgggacctgctggacctgaggctgggtcgggcctggctcctgggttcggcagtatcagtccctgccctggtcgctcttgcctggtagggagctgggaggtgac

SEQ ID NO: 110 - DNA, 2001 bp, Homo sapiens
cttgacaccccctccgggactgagccctcccagccagagcatcaatgtcattaggtcagcccatcagccctgggtagtcctcctctgccctctgggtcctctctgatggcatcaccagagtccacagatgtctgactccagccctctcgttctaaggctggggaatctgaggcccagccacggggggcggtgggtggggggagtttgccccaggccacaccaggagtctgggtggagaagcaggaatgcatgctctcagccctacattccacagctgccctgcctccagcacccccaccgcacccccctccccgggtcactacggacaggggagatgtagctctcccttccagctggctcatcctgctgcatccctctatcctccaacctcggtagctgccactcacctcgggtgggggtggagggctggagcctgcactgaaaggctgggctggtgggaagtcctgtcgggaagtgagcctggaggtgggatggccacctatataggcttctgctctggctattttgggaggtggcactccccgtagccccggccccctccccttccggtgtcaggtactccccaaccgtggtgctgggtgtctgtatcacattcagcctccctgggcccatcaggccaagactattaataactcttggcccctgcttccctccagcacccaaaattgaggcccaggcagggctcgcctcttccatgaagccttccgtgaccactgtagatggcagcgatgcttccttcctccatattctgccaggaaaggaagagaaacagcaaggaaaagaatgaaacaagaactgtcctctgtgcatggggctgagccccattcctatgctgatatgatctctcactacagccttgccaggtagacatagctgctctcgcacagatgaggaaacagcataaagacgttagatgttaagtgacctgctgctcaaagccacgggcctggggttgggagcggactcagcacttttgtgcgaggatctagtgcctggtagaaggaaatgttccgcatattctcagacaagtcccttcctctctctttctcagtctttccacctgtaacatgagctcaatcagaggtaaactgtgccctatagatggtctagggcgaggggaagatggaccccctgaaattgtatgcgtgtgacattgaggcacaaaggcccaagcccttcatgatttgcaaagagatctctgacattgtctgttttggattggagaggaccagctgagggatgtgttttgagaagcatgaatgaccaaatgaattcaataaatacgtagtgaatatctaatctgaccaccatgaaatgttcctctcctcatccctaacctgaaaagaatctccagggcacgaagattgtcaatggtggggtctagttcttcacagtgacacgaggaatcccaggctccatgcaaaggctgtttggaagctccatggatagaaagggccgagggcaggctgcatgggggaagctgggactggggcctccttgaggtgtggtgctggggcctgagctaggttgctggtggtggcaacaggaaggagtgtgcagataggggagcacctcttggagagaagcttccagaattggggctgtggcatgaacaagagggacaaatccaagctgaccctaaatattcaaggtggggtttgctgaagaaggaaggtgttgactttgaaggcagggagtgtagggaggaggtagtgctcagtcgaagggatgatagagtaagtggtgttgaatcctaggatgttagggctgggacagcctccgtggtgatcggctacaaacttctacaaatgacatcatgttatagatgaagaaaccaaggccagggtgacttacccaaatcccccagagagtagtggaagagccaagagctgatgctgggtttaggggacctgaaggacaccctggcggagatgtgtctgaactctggagtgaggtcagggctggaccctgggggctttg

SEQ ID NO: 111 - DNA, 2001 bp, Homo sapiens
caggatttcggcctcctctgctccgtgtgttggctacaggctaggttcaaatcccaattctgctctttactgctgaaaggctttgggcatgttcgttaatctcattttctcttctgaaaaagagacaatcttgaaaagattgaccttccgtgaagatcaaatgaagtaatgcgtgtagtgtgtctggcacgtcgtaagcgcatcacacacgtgagctgtggcaatactaaggagcagtggcaagagctgggggaggagtggagacgaacagacctggacaaattagtgacctttctgggacccctacagaactcttctggagaaattaattctatttttccatgtacttttaagtttgcttttcctagcatttcttctacattcctgaataattttccactcaaaaatttcactggttctccctgactcccatattaatctagacataattacttccccagaacagttctgtgggcagggagggctctacactatgcaccctccaaaatccccaaagctcggaccgtcctggcctgggtccccactgctgtcctgcccatgggctcccaggctgtgcttgctcttctcccaccccaaccccatcaccacaaaaagcccctcctccaacccggggacttttgtaatttcctacctgaaatcatccactccctacctctaacaatctgtgatgagcagaggacaacatcaagtttgaatttagaggtagggaatgggtgatttcaggtaggaaattacaacttcactttgaagtttaattttgtaataaacttcacttaattttgaagtaaatttgcaaagtcagcaaaattctttcaaagtcaaccttgctctggcctagttgcttatttactgctaagctggatgttttcattaaaagattaactttgagacttcagcctcttctttagcctgaactgtgggtagtgatgtggtgtgtttcctaacacctccggcagtgagccaggctttgagtggctgcgtctaaacacttctttccctgaggactggaagacattagaataaggtttgtcattgagtgtgttctgtgtgcttggaactgagaagggtgatgaagtgccagataggatgggaatggaagatatcgtggaagccaactatgagtttgcaaggcaaggaccttggcaaagatggtcctagggagacaggtgttagggaactcagagggcatgggcaaaggaagatttggtttcaaaaaggatacagacagagaaggttaagtctaggaagttgagtcttagaattgtatggtggaatattcgtgggtgtccataggaggaaaaatgggttaaaatgttttcacttgacagcgttctgaaaacctaaaaggtattgctattgccttcagggatatatttagtattctgaccactgcttcccggccacaagcaaaagaaaacagtgacatggctatgctcctaaaaaagtaaagaagttaatgtgtctcaaacaatgtagttcctttgagcaataagcttatttggaacaaatgggttacaaagtcccactaatgaagagcctctgattgtgttaagttgaatctacagatcaacctgcggagaactgccaagaacgctaatgaagacagggaggtactggtataaagatagataacagatcaatggaacagaataaagcgtccattagcaggaagcaaaatccgcaaacttttattcttaactactactatattctgcattttacaagatccttcaattctcacaagatccctatgagatagacagtaataacctaatttaaagatagatgagaattggagcttgtctagaatagattgagtggaacatcacttgtggtatatcatttgagaggtaatgggaatattgccaaattcccaggtaactctcccctcaaagggactcctgttgtactctaaatcgtatcagctatcaaatgatgagctatgaaagtctgtcttcttcgactgtaagcttcttgaga

SEQ ID NO: 112 - DNA, 2001 bp, Homo sapiens
cttttgacaccctaaggtttacaaaaatccattaaaacagccatcagagatccctgtcacaaattaatttttccattatataagacatatggctatagcctaaagtaatgcctcccataaattatgatgaaccatatggagtaaattcatagttatcgaacactttagcttcaaaacacacttaactattaagataaagtgatttctttcctaatatcttaaccaaataatttttaaagactcgttcaagtaatattaactatccaaagagttagttttatagaagaataaagatgctacaattatttttaactttacacaaggaattactcttcaacagatgacttccacagccaaggacacaaaggatgatactattctaagccagtgctaataagcctgacatttcttcaagcttcactattaaactctgattcaaattcgtttgtaaataaaagacaagatcataattaggtacatacaggtaacttcttctccagacgaaatctttccatagtaatatagaaatgccttgtttctgatgatgtaatgcagtaactcccttgaagattccagggacagtgagcacatatctgatttaggtcactgtgctttgcagagatgaattcttctaagttacttttatggtacagccaatcactgtgttctaagttacctaatcttccaagctaataactacaacttacacagggtcacagcataacagatttggaaagtaatatacacatttagttataagtaaaaataccaagaggaatgcagcaatccgttttcaactgaaacctttctgcaatgttagtacaaagagtgcatactctaagaatatatgtggatggatgtgtgagagtatggtttataattatcattgtaaccaaaaaacaaagtattaatatggacaatagctagaaaaaaaaaagagtagcacccctctaactactggacttgggattctctacattcttaaaaattcctgaagaccgaaaaggaccttggtttatataagttaccacatcaggattaaaacagaaaacttcaaagtacttattcagaattatttacacaataataaagccattacatgttaatatacataacattttaatgaaaataacttttcaaaaacaagacaaaacttagaggaatacctctgttttacatttttacaaatctcttcaattatctaacttaatagaagacaactggattctcatatctacttctgcagtcaatctgatgtgatctgcccttggttaaagtatatttaaaaatccatattcacacagatattctgctggaaaggggaggcatatttcaatattctcttcaggtagttatggacattcctctgatagttcatcaaactcaacaagtatattgtttcttaatagttgcaacgtagaatctgaaaccattccaaatgaacattttacacattgttacatcaaaatccatttgtctactatgcactttgaatggatctctcacatttttggaatgtatgtgttcacagggtattagagcttttccaaatgtcacatttcattaaacaatatcaaaaatttttttaaatcacacttgttaataatatcaccaacaatctcataagtccaagtattgagaaactgtcaaagtcaggttagcagatgcaagttttccaaaattctaagttttgcctgaaagtctgaattattatcatgggtaacaaacactatcagttgtttttcttgaagtgacaagttcacttcattcatttttgacaaaatatctgtcaaatgcccaagtatgattaaccaagtctgtcattttttctttcaggtaaaaatggtgttccatgaaaataacagctagttgagttcacaattcaggcaattaaccagtgatttttctccaaacaatcattctatttggatatgcagcagaaatgtttttacaaatgcttattttgtcattcagaatattaaagcaatacacaaaggcaaa

SEQ ID NO: 113 - DNA, 2001 bp, Homo sapiens
ggagccagagttgcctttagctctgcagggattctcagccaagaatcagaattttctgtctgtgattttcctcaaataaacccagacctattgaatgagaatctctgcttatcagacctgtgcatccttttcgcctcttacatagccccacaggtgattctgaggcccacgcggcccgcttcccctccctgcgtcaggcccggccctagctctggaggtggttggattggttcaagggctgcagccccacctcgcgtcttcccaccgcgtcggaggacgtaggcgcctgggatcctggaaggcggccagcctctgcagccgggcccagcggctcctgagactggggcggacctcacacctgaccccggagcggctaatcctgcccaaccgtcccacctgccacgtgccctcggggctggaacgcggcgccccgggagaggcctgctggtcagggggcgctggtgcgcaggcgcgcggggccgccgtcgcagctaccttcctgcgccgattcggagagggccccctaatctctactcggcccgcaccagcagcgctggcgtcagggcgcggtgggggcaagacgggtgactggcagcagggcggtgccgggccgagaagggcgcctcacctggcgcctcctgccttcctttccacctcaggttgcagagagccgccagggcaggtggaagggccacgccccgctcccgtgagggtcaccgagagacgttgggggtggggcagggaccccaaccctagaccaggtagggggcgggcgaagattggtggtaccttcaggtaagagggagcggggagcgccccctcggcttggggtgccagaggtgtgcggcgggagcgccttttttcgtggggccctcgccctgcagggcggaggcggcattgcgttggccttagttcctagtcctggttcccgaggagcccctgaaggcttcggatgggaagtttgcgttggagtccaacacccgacaggaaccccagttaacgcctccgcatggccaggggcagagttcttgacacctgtagcttaatcaacgcaggtggagattcagggcttgaatcgcccggccacgcgcaagccgcagggcaccctcatactactacttctgccttattacccttaaaactggagtctcagttatttgtatcccactattggtttttttttaaatcttttttcgtgggtttgcccccttttcctcagctgcgtttctgtcttcagtcctgtagggcacaatgtctgaacaagagcgtatacaggaatgtctgcggaaggaaataaggtcacttctcatttccaccaaagatggtttgagcccacaggagttggagaaggagtaccttttgatggttggcaaccatctaccactccgaatccttgggtatcggtccactatggagctggtattggacatgcctgatgttgttcgtgtctgccccggtgcaggtggtactgtaatactgaaaggtaggtttaagatttttgaaggtctataaactttgtaataaaaacaattgcttagtaaataattattctagttctgcagttattatgcatcccagccagtggattttaatttggtgggggagggagactcataggtgcttttgaggttgtgggcaaaattggaacctctgcaaaattaacaaaccattcccccaaacaatcgtgcactcacaaaagctacatttttacatatgatttcaggttcttgcaccttaagtcgagagaaacaaaaaaaggacttcttaattataaaaacatgttttaaatttcgtgaagcacaagaaaaaactattgtagggaagtttgcagttcatcccaagagtttcaaaaaatctgtcaggggtcaattagttgtaatgataatacattgagttaacttttcttcagctggcaggagagctagcaagctgttgcccaaaatgcaagaaatcgaggaaccaagagttactgtttatcgtgctgcctggataaacagctcc

SEQ ID NO: 114 - DNA, 2001 bp, Homo sapiens
ggtagctacggaaacgtggagaggacccactcactccgtccagcagtaggacttgtcatcatccacacgaacaacaccacaggtagggttgtttgtgatcattcacgcacacacacatttagccctccagaatttgaccaccaaggaagtaccttaccggctccctcggcttcttcctttgtctgtgcacagagttgtctctgcagtgtgtgaggatgctttaccctaggttgcctgctggtttcttttcacattgctgagagctcaggttatttcttgcactgggtgagtcctgatttctcacccctgaggccatcacaagagggtggggcatacctcctcaggagagagaaccagagactgcccctgaaggggaatgtaatcacgagcaagcccccaaattgttataaataaagtttccccccagtgccgcaaaagaaatagcactcgaatataaaattttcttttttttcttctcagcaaggcaatttacttctatagaagggtgcgccctcacagacggagcaaaggtgagcgcacacctggacaagggaggggaaggtggttcttatgcctgacccacgtgattcctgatgctgtgtccttcccctgttggctagggttagacctataggctaaactaattccgattggctaatttaaagacagtgagggggtgagtggtttggcgggaaaaatggttaccgcagagcaggaaatcggaatgaaccacggtgaagaatgagtgagggaggagcaggtaatcgaaaaatgttgctttatgaggaagttaagtttaaaagtagaaggcaaagaattgaagatactgacatattgattcttttaagataaatttagaactcatatctaacagctggcatgtctggcatggggctccggctggggcgcagtcttcagcatttggggctctggggtgcagggggcagaggacggccggggctgtggtgcccaaccggtgctaaactcaagcttgggaggtcgcccacgagaaacagtcgcccttccccacccgcctgctcggcttggcggggagcgggatgtcgctgggggcgatgccaggcgggccgcctctgatagcctctaggctctggcgcgcttccccgcccctcccgcgcaggcgggcgtgggatcccggctgatccagtccgggttttgcggcggagcgggcgggctgcgcgtgcggcggcttcaactgtcgcggtaggcagcagcaggctgaagggatcatggtgcagtacgagctgtgggccgcgctgcctggcgcctccggggtcgccctggcctgctgcttcgtggcggcggccgtggccctgcgctggtccgggcgccggacggcgcggggcgcggtggtccgggcgcgacagaggcagcgagcgggcctggagaacatggacagggcggcgcagcgcttccggctccaggtgactgccggagcgtagtgggatgggcgcggcctgagggtactcgcagcggcactttcagccgccggacccgccgagggacaggggatggggcgggcagagcagatgtgtaactctccagaatctctccttgccctaagatattccgcactttctgagccaggtagagcacgggggaagaaaggaaacggtagtgtcaaggcaggtggtggaaggcctttccctcactgggtcaaagaggagttttgctttgtgccaaagaatctcacgattgctgcctattggaatgcgaaaacgcaagcgtgacaaccagtgggatgccaggggttgaggactttggaatcgcagagtgtaatggccaaggggatctggagagcttagagttttgttttttgtttttttttgttttgttttgagacggagtctcgctctgactcccaggcaagagtgcagtggtgcgatctcggctcactgcaacctccgcctcccgggtgcaagcgattcttgctgcctcagcatccccagtagctgggattacaggggcccgccactacgcctg

SEQ ID NO: 115 - DNA, 2001 bp, Homo sapiens
acattaaatggtctccttatggttaggataacaaaaacatgaagaaagcatataagattccaactaatttgacgttgtgaattgtgaattctgaactgaaaccaagtaagtcccttatgatcaggctccaaggttttggtcagcctctcaaaattgagaggctgaaaccctttgaccaaaattaacacatttgccccttccctccagctcctggaaaccactaccctaactctgtttccataaaatagtaaactattttagagtccacatataagtgagatcatgtagtacccaccgtgggcttattttaaggctctgagtaaatcaccatctctctgtgctagggtattctgagtcagtcttgttataaaatattttgtttaggtaacatattgcaaaacgtttcataattcaatggttcctttcccagaaaaaaagcagtgaaagttccaatagccaaaaagtgtctaactcaactgacctttgggaaaaaatatccgtataattccagatagtcctaacttctgaaggtaatatagcctggcctctgttgataaacaaacaaaaacaaaaaaaccacacacacacacacaaaccagcttcttgtctttaagtgtgcaagaatgttagcacttaaaatttttaagatacacaaaaactaaacagaactgttcaagtttttaaagggagcttggaaatttgaagaaagtaaagttaaaaatattgccccatgagtcatataattatttttctttttatttctcatagcttccctcctatccattatttaggcaattttgtatccaaatttaataatttaacaccttatttgttacaagaacacgttactatgttattttaagcttttttgggcaaattacacataattagataggctgggttagggctgtctacaagatcccagcctagagtgtggtgcagtagcctattttctctaggactcttgctgcaacaggatatgggagaagggaaacattaacgttagcagaatgggaggaatcagattatggagacatgatccttgccttctggcacctctccagctgatttagaagaggacacagagggagacagacatatggccatttctataattataaactctgctacatgctgagaggatggcttctcaaagccagttagtgtacaagcagtggaggttgggactgccctacttctcctgcctagggtcacacagtctggcagatctggaaagacttccctgagaaagtgaccttggaactgataccttagtggaaataactaggcattgggagggggtgaggtgaggagaaggtcaagagctttcatgtagagggaactttgtgcagagaccttagtcaggaagaggttttgcatagtggaggaactgaaagctaacagccaggctacagtacagagcaaatgatgctggggtgtgaggtgatgtcaaaaggtagacaccagggctagagggcgcataatcttgcaggccgtgctaatgattttgttttctacccaagaatgctacttcaggagatcagtcaaaaggttagggtgatggtgacggtggaaatatagaaaaaggaatttattcaagaggtattcaaaaagaagtgaaatagagatgagttggagatagattggataaaaagagtgagggagagggaggtgtcaggctgcagcccaggttctgagttgccaaagaggagtcttttcctccaggtgagctaaggcttagaaccacgatttttggtcactttttcttattttcagtgtgagaagtggcgagttggctgtccaggtacacactgtcatcttacacttactgtttaagacagaggcgcatatttggaaagtgaatgagtcaggagccagaggagaaggtatgccggcagagcctagggcggagaggaggaagagagtctgggggcgcgtccccaaaaaggagacagggaagacagagcagaggccggggaggggagagcaaggaccatgaca

SEQ ID NO: 116 - DNA, 2001 bp, Homo sapiens
ccgggacctcatccttaaccccttcctgggtctcagctacccccaccctttgtctctgtcccctgcagccactggaggggtccctctcaaacttcactctatgtggatctctgcattttccaaagtgcttctagtaagctcccccttggccagaattactggcaaaacaaaattcatatccctaaaaaataggctttcatgctgcgttaattatgaataaaaaattaataaaataagctcttcctcagaagtaaaaaagattaaagacacaaataaatataaatatttttttttaaaagttctaggagctgggagaatggcttgagcccaggagtttgagaccagcctgggcaacatagtgagaccctgtctctaaaaaaatgaaaaaaaaaaattagctgggtgtggtggcacgtgtctgtagtcccaactacccaggaggctgaggcaggaggatggcttgagcccaggagtcaccccagcctggatgacagagtgagaccaatcaactaacagggggcacctcccctactcaagcaccatccatggctccccactgcccaccagatcaagatccagccattcaaactggccttcacagtcaactcctgcatccctccagcctccacacctctcacactccaaggccagggaaagccatctgctaagtcacaccctccagtttctctgtgcttgagctctaccccctctcctgcacttgttgaaatccaacctctccatgcagtcccagctcaagtgtcacatcctccacgtcgccttccctgacctcatgtctgcctcctccaagcagcaaaacttcaattacttgtgtctcctcttcttcaccaggagacatgccccacacctcccacgccctctttccaagggcccagcaccaaatgagagcgtattcttagaaaaaagagggataaggaagtcttctaagtttaccacctgctggggcaagtggagattcttgtcttctcatctgtctgcccacgtgctaaggtcagccacacatccacccaccgactccggaggacagacacatccagaggcggagctgactctgtctccacacatctggagtagacgggttggccaaggacaacatcctggtcagagggtcaaggtgtctggcagtgggtggggaggagtggcggagggcgggttggcggggagcagaaccagtctggtgctgcttgttgaggttggaggtcttcctggaggaagaggctgcagagcctccaggggtgggtgccggggaagcttaggcccaggatccagagagaggggaagggctggttcccaagggctggtggagccagagaattcctgagggccccagaggaggcttctcgggggggtcagtggggccccagggggtgatggtttagagatcgctgtccaagcttgggagcaaacctggaagtaagggccatggggggtgttggtgtgggaggagtgtggtccagagcgggacttgggcgctaatgcggcccagcagtggcaggagggcggggagcctgatggtttgggacttctaggtgtccaagtctctgtctctcttcatctctccatctcagtctcagtctctctctctttccatcgctctccacccctccacacgcctatttcttgttctctccttttctagggggcagcgctggagtccccgcccagaaccctgcgggagcccggggcgggatgtccgggggcggacccctcggcacggccgggcggggttaaagggttgggccgtgggacggcgcggtcagatcccaggtagtggcgccggtggatccggatcgagggcaggaggctgagacccgcgggtgagtgcgcgcgtggtgagggctgtaacctgagcgcggccgcggcgctgcgctggaacccgaggctcgcgcggttcggtgctggcggggcaagggcgaaggtctgcgcgcggcggcgggcggggcgcagccggccacggggacccccccaggacccaggacgggg

SEQ ID NO: 117 - DNA, 2001 bp, Homo sapiens
ctccaggatggacttttatatcttgataagcctgggggtcaccatgctgcgtagggaggccggagacctgccactgacggtggttatcccatgtcatggctttcttcattcccactttacctttttgaggctggaaggtatttaggggatgacagaaaatcaagtttgaaggtcaatggaaatattgggcacccactagaatgtcccacatctgtaaatacatgtgcatctttgtaaacacttaacagctgttttggggagtgcttgtttaaatccacagatggcattttgccagaggcctccttcagttccctatttctctcactcaaggtatcctagtttggagatccagaccaggtgcagcccatgtaccctgatgaaccccaggttcccctctccaacatctttctcacccgacaggtgcggccctgaaatgggtttctactgtctcctcacacagtccccatgttcccctcctcgggttagttttagggtagtctagcctgggaaggaatttgggactgatcatgccacgttccctggaatgggactagaaaggaatcctgggcttcgtgcggaccaaggccccactcaggttgtcatggaaaatgaggctgagagcccctctctgcagcaccctgtgggacggaacccgaggtctctgctggtcccggtgggcacagacctgcagccaacccggtgttccgtgaagccctgatttcccaatgaagcccatggaagccatcactggtttccatgggagcggaccatggccacccctgctcctgttccctgtgggaaaggaccaaggccgcccctggtcctggtcactgtgggaagggactgtggtcaccgctggtcctggtccccttgggagggactgtggtcaccgctggtcctggtccccgtgagaatggtctgtggtcaccgctgcctgttccctgtggaagggaacgtggtcactgctggtcctagtcctcttgggagggactgtggtcaccgctggtcctggtccccgtgtgagggactgtggtcaccgatggtcctggtccccgtggaagggactgtggtcaccgctggtcctggtccaggtggaaacagaccgcggctaccctggtgatggaccccacggggcacacccgcgccaccccgcacccagggctcgcgccgcccgaggcccccggttcctgtccccctccccgcctggcgccggaacctgcgagctggggcgcggcctcggggaggggcgggcgggacagacccagccgccccggctcccccgccgtccgcgtctgcgccggccccggggcctggtcggcggcggcggggccggtcgatggcccgggcggcggcggcggcatgcggctcctgttcctggcggtgctgcggccacacaccggcaacgcggtcacggcccagcgcgttcggtaggtgcagggcgccggggcctacgaagcctgggccgggggccggggggcggggacgcgggacccggggacgcggggcgctcagccaggccccctccagccgcgccggggccgtcccgagccgcgcgcacaaacggatgggccggtgccgcctgccgggcgcgcgggggtcggtgccttctgcgtggcgcgcgtgtccccgggtctccgtgcggccggcgcattggcctcgcgctctccggaggggactgagcaggtgaacaggcccggagcctgtcgtggaggggcccgggaaggcctcgctgcagaagcaggatgggagcaggatccgcagggatgcgcagcggggtccgcggagctcccggcgggggcgcgtttccaggccggggaccgcggtgccagccctgccctcgcggatcgggtctcggctgtgtgccggcgtccggggtcctgtccccgaagtccctttcccacctcccctctgccctgtctattctcgtcttcctggctggttagctccttcacactcatctttttctgcctttgtcttatttgtttacttgttggtctgtgt

SEQ ID NO: 118 - DNA, 2001 bp, Homo sapiens
tgtgtgagccggggccttggccaggggaaggacgctgctcagagctcacagacctgggtgcccgggccctgacgactcacacaggcccggagctgggagaggatatggtgcagggtgtgaaggggctggtccaagacatcccccagggctgggtcagtgtcagcggtggcctccagaaccttcagcgtcagggccagctcagcctccaaagccacggggcgctccctcacctgaggagaggtgagaaagagcaggtgaggggggaggtgaggggaacaggttgggggaggaggatagagaggaacaagtgaaggtgacaggcacaggggagagggcacagccagtgtggtcaggtaggagcagagggaaggggtagcaggtgtggggagaggagagagggacaatggagaaggagaaggtgaaggggccactacagagccaggtgagcagggctgggagggcaggggtgggcctgactccccctctcacctgcagctgcctcaggtcccaggtcctggggaagaggcgggagcggcacttgcagtccttcagcagaagcgactcttcctagacagcaaaggcacaggttagccccagcaggaggggtggaggttagaccactctgatgggattggaggatggctgacaacaggataggggaggttaactgctgggaggatggtagaggaccctcttcttcaggaaaacatgagtcagtccctgcagtaggagcatgagatagcccactgcagggagggtggaggctagcccagtgaaggaagctgggagtgggaaagcatggtgacccttggagtgcgggtggaggctagtccatggcaggagggcagggggagactcactaaggcatctttggccctcttaaaggcctgcagctcctgtggagacagggacttgaactgggctatgtggcagccccttgcatccgggagagccccgcggagcctggcgacaggaactgctccagtcacggtcagcactgcggccatcagcaccagcactggcatgcagtccccggtcatgtctgtgtcacagagagaaagggagctgagggaatgcagaggctgcccactgagggcaggggctgcaggagctgagcacggacagagatgtgggactcacctagtttcattcctgatctctggtctttgtcagcagaagaaacactctgaggctgtcacccagggtctgtttgggtcttgtctggagtctctgttcttttcagtcccctcttctggatctcagactgtgtcctggctttgactctttctgtcagttttgagctctgtctgggatgtaattcctgcctgagctccatggggcagcttttatccctgacagaagggcagtcccagctgatgtaggaaaagtgaaaacacagcctcaggtaagacaccggccaccaggggagccccaggctgggaaagcccagagcagggcagggctagtgagccagatgagcagctggagggaaagagaaactgatggagactcaggggtaacctacaggaaggtatgttcccaagaggattccacctgctctggttttgttgttgtttaagacagggtctcactctgtcactgcagtgcaatggcatgatcacagctcactgcagcctcaaactcctgggctcaagccatcctcctcacccagcctattaaagtgctgagattacaggcctgagccaccactcctggcatatatatatatatatatatatatatattttgagacagggtcttgttcggtcacccaggctggagtgcagtgcaaaatcttggctaactgcaacctccgcctcctgggttcaagcaattctcttgccttagcctcccaagtagctgagattacagatttgtgccaccattcccagctaatttttatatttttagtagagatggcgtttcaccatgttggcaaggctggtctcaaattcctgatgtcaagtcatctctctgtctaggcctcccaaattgctggga

SEQ ID NO: 119 - DNA, 2001 bp, Homo sapiens
ggtggatcacctgaagtcaggagttcgagaccagcctggccaacatggcgaaacccccctctactaaaaatacaaaattagctgggcgtggttcgggtgcccgtgatcccagctacttgggaggctgaggagggagaatcgcttgagcctgggaggcagaggttgcagtgagccgagattgtgccactgcactccagcctggcgacagagcaagactctgtctcaaaaaaaaaaaaaaaaaaaaaaaaagcaagacaaaaggacttctctcctgtcacagactctgccctttctgagagtctgggaccctactattgtcactcctaaattttgtttgtttgtttgtttgtttgtttgtttgtttgtttttgagacagagtttcactctgtcgcccaggctggagtgcagtggtgtgttctcggctcactgcaagccctgcctcccgggttcacgccattctcctgcctcagcctccccagtagctgggaccacaggcgcccgccaccacgcccggctaattttttgtatttttagtagagatggggtttcaccgtgttagccaggatggtctcgatctcctgacctcgtgatctgcccgcctcggcctcccaaagtgctgggattacagacgtgagccatcgcgcccagtctgtcactcctaacttctaatctgctctagggagagtcccaatagtccatcttgcatactgaagattaagtgaatttcacagccaacaccacacaataccttctctattgagctgtaagctgcctctggactggatggtgttttgtttttatttttaccgtcagcaccagtgcctctcacatagaatttagccaagccggttaggtgttaagtgctgctgctgcgctgtcagaagggcacacggggctggttctagaccggcctctgctgcttactcgttcagtaatcttctacagcaagttcttgaacttctctgggcctgttttctacctcgccgagtggttaactgcgtttaggatcttggaagcacctaagcacgaaaagcacataggccagaagttaagatgtcccttggggacgcaaataccgggaatttctccggctgttcccaccgatcaagcaccagggctctcaggtctagctccttcgtctccccacccccacatataccctcactttttcttcctttccctatgcctcgcagtccctcaacagggtctgcggtcagacgtcactcccaggtgactggcaatcagggatgccgcctcgctcacctggccacccttgaagattgtggatcctgcaccggctagccggctggcagagggcgcgccaacagccgccagccgaagtgcccccgcgtaggtgggaggagtgactgcctctcaccatcccccggggatgcacctgtggagagacggcccgagagttccagggtgagtgcgcagaggagcttatctgcctgcctgcgggtcggtgtgtcctgccggctgctgggcgctggcctgcgtcgcctgcttgtctcgctccctgtccctcgtccgctaccccctctgccccccctccctccagcagccggcctctcggtcatcccgccgggtgggtgcctgcgaacctcactcctccctcctctgcaggagccaactggcgagccgcaggccgccgggctcggcctgggagcccataaaacccgcccgagccgcgccgctgcgctccagccgctgctcaccaggcagagcgagcgggcttggctggggcttcctgccctgagcgcgacaccgactgccccggaccgcgcctccaagcaggctgaagggcttccgctcttggcttccagaaaagtttggagaaagagaatttgaggcggattggagggtggtagcccctccccagccttctttccctcccagaagcctcactctgcacagcgtcccccattcttcccgtcctgattccccatcttcctgacccctccttctccctctcctgggtcgatcccagtcacattttc

SEQ ID NO: 120 - DNA, 2001 bp, Homo sapiens
gtagcggggagcaagtcttgctctgacagggagcagagcagcaggcggcagagtccagggccccacaccctcatgctcacttccccgctgcccatgcacagagcatagcacatgcacctgagccacctggcactcctcaatctcagcaaagattttcaagggccacgaccccagtgagtacaagggagcctctgccctgcggcttttctgtatctggaaacaacactgctgaggcctccagagctgggtctgctgcttattatctgtgtgaccctgagcaagtcacctcacccctgtgtgcctcagctttctcattcataaaagtaggagggattggaccttcctcatctggatgctgccatgatgaaataaagcatgtgggacagtgtcgggtgcaaagatgccgctgggctcccaactcagtgctcattctctaccaaaatgaataggaggagggtgaccataattgatgctcctaactttggggcatttttgaaagtaaaagggggcattgttaatgatgccagcactgcaaacataaaccagtacagtcctgggcaaacaccatgtggagccaccatagtgccagcaatatcatctctgtggagacaggactcgggccccaccctaagatccagggactcccaagggaggctggaaaccctggggttttccaaaagaggctggggcactgcaggcttttattagttcctagtcaggacacactagtaaataagacaatgtgtccttatccccagggcacagcccagccccaccccagccgcagacccacctgctttggcagaggctatttactgcaatggtatttgtaatagaatgaaataaaaaactagaaacagccttaatttttatcagagtaggactgataaaatatattacagtacatatactattcagctgtttttaaaatgagagagcaaggcagctctatatcgactgatgggagaaatgtcgtattttgctaagtgaaaaaaagcaacgtgcagaagagtgtagatagcatacttccacaggttaagactatatagttgaatatgaacagacgattcaggaaggaaaacaagaaactagggaccctggtgttcaggtaggcagctccggttgcagtctgcccctctgagtcactccaggcggtgacctcccccaacccgccctcattcctgaacttctttcctggcacaggactcactgtgccccttcccgctgtgggtacaaggtctgccccccaccccagctctccaaagcccaccggcctccctggaggccgaggtcgacggcccgtcgcaccgggagggggggctcccaggggtgccccacgcacggtcaaggtcccgcgccaagcggggaccgggctgggccggaagcgggcacggtactcgcggcaaactagcgtgggcgagtcctgattgcagtcggacctgccgccgcggcacttaacagtttgcagagtgcttcccgcccctgatctcattggagccttcggacagcccagcccatggccaccgatgcccccatttcacgcctgaggaagcggaggctcagacgggccaccagcccctccggaggctggcccgggagcgcctggcagcgtcgggtctaggagccggctccctcctgctccctcctccgcgccgcccggggtgtgcccgccgtctgtgtgcaccactgctgagcccagctccggcgccctcgcctctgctgtgggccccggggacgcggggtcaggccaccgcgttggccaggccgctgcaggtaagtgctggacggggcggggcgaggctgggcgggaccctcaggggcgccaagcgctcctggcgaccccctgcggcagggctggggcttgagcctgccagtgcgctgggatggggcgcgaagcgggggtgcggggagcccgccaccccgtacctctcaatcctccctctcgcctctcaattctgggcccattaggggtcccggctgcgggaaggttccgagacaccc

SEQ ID NO: 121 - DNA, 2001 bp, Homo sapiens
tatgatggggtcttgggtcacaccatatagcctatgcaaatagtgtgatttgtagctagggctgtcttagttcatttgtgctgctataacaaaatacccaagactgggtaatttataaataacagaattctttctcatagttaaggaggctaggaagtccaagatcaaggtgacagcaggttcagtgtctactgagggctgccctctccacttctagatggtgcctagctgctgtgtcctcacatggcagggtcggagggcagatgggagggagacagctcccttgtacctctttcataagggcattaatcccattcatgagagatctactctcatgacttaatcacctcctaaggtttcaccttttttttttttttttttgagacagagtctcactcttgtcacccaggctggagtgcaatggcatgatctcagctcactgcaacctctatctcccaggttcaagcaattctcctgcctcagcctcccgagtagttgagactacagacgcctgccaccacacctggctaatttttgtatttttagtagagatggggtttcaccatgttggccaggctggtctcgaactcctgacttcaggtagtctgcccgtcttggcctcccaaagtgctgggattacaggcgtgaaccaccgaacctggccaggtttcacctcttaataatatcacattggtgattacatttcagcgtatgaactacggggacacattcagagcatagcaggggcctagggccatgcgatatcagcttgacctctggaggggctaagactcaaccagtcacatttgcacttgaccatgtctccaagacaaatctccagtggaatctctggacatcaaggcccaggtgagcttccctgattggcagtactccagctgtcttttcatacatcttgctgaaagaagtaagagctgtccgtacaactcccctgggagacgacagctggaggcccctggcttgttgactgctggaccctgccgtatgtacttgtgccctgggctgactgtagtctgcattttttgctgtatagactctaaccatgagtataagtggcttgactgagttctgagtccttctagtgaattgtaaacctggggatggttttggggatccctgaccttgcagttagtgtcagaagtgatggtggccactgggaggccgaggcaggcggatcacgaggtcaggagattgagaccatcctggctaacaaggtgaaaccgtgtctctactaaaaatacaaaaaaaaaaaaaaaaaataggcgtggtggtgggcgcctgtagtcccagctactcgggaggctgaggcaggagaatgatgtcaaccctggaggcagagcttgcagtgagctgagatcacaccactgcacaccagcctgggtgacagagcgagactccatctcaaaaaaaaagaagtgatgatggaaccctcaaactttgtatttcataaagcctaaaatctatttattcttgcatcagctacttaaaagttgttcctaagttggtgcaaataaggcttttgagagaatcttttcttattacttctccttggagaatgtaattgttgttgtgtaattggtatgaaggtttaggcctggaatggactatttttagtaaattagatataactcttttctaaatcttcttcttttattcaatgtttgaaactttgatttgttaaacacacaagaaagtggttcactttttgaaaagagcttgacttaacaagtgaagaagctggtagagtgtggtaagtaccctctcccatctggctacaatggcaatggagaatgttatagaacaatcactcgttagcacagtctgagtttgcaccaatttgaatactgaattcaaatactcagatatacctgaagattcctctcctagaataaaaatgtcagaatctctgcaaggaactaaggatatcagagctctggtccaagatgaagagtctatcaccaagtgcctcctccccacagaaag

SEQ ID NO: 122 - DNA, 2001 bp, Homo sapiens
gtcacctcccagctccctaccaggcaagagcgaccagggcagggactgatactgccgaacccaggagccaggcccgacccagcctcaggtccagcaggtcccgcctgtccacctgggccaggcctagagcccgggagcccctggctggtgggaggccacccgcaacccaccccacacgcagctccagctcccccaccaggcggggcgactaggacagggacagaacccgttgaacccaggagtgagatccggccccgggtcccgctgggccctcccgtccaccttggctggacctggcgcctgggagaccttggctggcgcgaggccacgcccaccagacatgcagttccagctaccccaccagctgggcgaccaggacagggacggaggctgctgagcccagttagaggcctgccccccggggtctgtcctgggcgctcccccaaggacggacagggcaggcagggtccgggacgatggccgcacagtcccggccccgtgttcccaggcccgtcttgctcctcgatgtgagggagacccgggggatgggacaggctgggccccgcagtgcctgactccctgcagggctcccgggacaggggtccggcggacagccggctgctcaggggtgaggggtccaagctggcattgcggccaccttccggcccgggctctcttggggaggggcggggttggtgagaaccggtcacgtgctccggggctcactcggggtctcccagggccggaagtagggcccctgtgcgcaggcgccctgaggatcccgggctgcccatctcacgccagggggcggaacttcctgcagcctctctgcctccgcatcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttgtattctgttctgttctgttctgttctgacagttctggtggcgaggtgggggccgggagatggggagggcagggccaggtgggggaggaggcgggggagatgcgagtaagtggttgggtgggggtgggggtgggggttgggaggtggggatatgagaggccagctgcgaggaggggaggaggtaagggctgggaggtagagggggtgggggtgggggagaggggctgggtgattgggtgaggggcaccgggtcatgcggggaggggagcaggggccaggagatagggggacccaggtgtaggaggggatcaggaggtggggggaagggggcaagatgggagcgggtggcgggaggtggttgggatggagggtcgagtgatgaggagggggccgggtgatgccacagggggcagggtggggaggggtcttgtaagggcatggaggagtcagcttggggtgcagcaaaggaaagtggggaaccaagacagctgggtggggtgtgctactgggccagtgcctggggagacagcctgggggggatgggctgggcaatccggggtaggggggaggggtgggggagtgggggagggggaagctggcctggggtaagcagcaggccggagccacaggccaagtcctaggagaggatgccttaactgggcccccaccagctacctcgccatgcctttcgcgacacccatggaagcagagctggcccgcaggagcctggcccaggatgccccaccgcttcccgtgccaggggtgcttctgaaggagttcactgtgtccggcaacatactgactatgtcagttcaggaccaggacagggatggggcctggg

SEQ ID NO: 123 - DNA, 2001 bp, Homo sapiens
gacgcgcgctcccctgcgcccccggggcctcccgtgggctccgtgcggggacaaagccagcgccagcaggaagagtgcgggcaaaggggcgccgggcttaaggggccgcatgttcgcaagccgggaggagagagcgggagactccgggaggatcccgacgcaggtccggagggtgcgcggcccaagagaaggccagcgggaccacagcgcggctacgcggccggccgcagtcttcaccgcgcgcctgcccttgtctacgtcccgggggtcggctggagctgcactgggactcggtcctcagtgtgccgaagcctaagcgctgcggggcgcggggcgggaacgggaggcggtgcctggggccacggggtcgtcccccaggatgagggcgtgtcccagcgcgcgggaccctcggaagtccgcgctgggccgggcgggcaccagcctcggactcagcgggtctcagggctccctgcgcaacgcctgcctcggatccggaccccgggctcgctctctggtcgccgtccccgggaggacccagtagggtaactgccgcgtcgccccggcggttctccctgggctctgtctcccgccgcctccaccccccgagcctcggggtccgtcacggcttcccctggctggcggggtcagtagaacccgcggcgcctaggtccggacggaaaaaagcagggccggggtgcggcctggatgagcggagatctccgcgccttgggctcaaaggtgcggggtgcgctctgctgccgagcccctgctcgctcaggaacactggccacgccgtcacgccagccgcccctgccccaggtctggaggcccgacctgctctcctaggcgcagcaccgcgttctcttccgcgtgggggagcggcgggcggaagaggtctggggctgggcaccggggacacgcgcccagctcccctggcctccctggggggagtggccggtttcagtgcttccccaggtgaaatcgccaaggttgaccagtgcgcccaaaaaagcagccggctggatttttcttcaaattagggaactcttataaagagttttagtgttaacaacttaaaaaatgaaattaaggccagtcgttctgattaaagtgcgcctctctgatcaaaccgcgctctgtaaactgttgttgttttagttgtttcagccgcctgctttattgctttatgttaagtatattgaaaaatgttgaaattgctcgctaatcaattgactactaattatctcctcatatgcaaagtgcaaacaggtgaaaggaaaaaggcaggaataactaagggaactgaggctccattactccttaggcggtgcccaacgttacattactctacaaaaagaaatcttgtgggtcacagatcctgaataatgacctcaactccttcaaatagtttatctaggcttggcatgtccaacatgcctaagaaattgggaagcaaagcaagctttgcaggagccctgtgaaatcgtgcctttggatgaagctcttttgaggaagttgttacacaggtgaagactgcttcttccaatcgtttcactgttaagcacgcataggaatctggtatttcctaagcaaacacagcagtgtaagagaatggcttccaccaagggagacccacaagagtcttcctagaaaaacaggttaagggtctgaaggccctacttaggtggaataaacgctttctcacagctctactgaagtgaaataagaaaacattagaagttcacagacattgacaggcagttcaaaaattaattttaattggaaaataaaaaaatagggactagtccgaaaaacgatggggaaatgcagtatgaaataatgctggagaataagtgatactgaaaccatgacaaatggactttttaatcaaccatctatccggagcaaaagacagcaaaacctggaagctgaattatttccctcttcctctacgttgaaagctagaggaaataaacaaagcaatgaagttttcaagcctga

SEQ ID NO: 124 - DNA, 2001 bp, Homo sapiens
agagcattcctggagctcaagctcctctacaaagaggtggacagagaagacagcagagaccatgggacccccctcagcccctccctgcagattgcatgtcccctggaaggaggtcctgctcacaggtgaggggaggactccctcggagtggatgggaggagggagcacagagactggctagggtctcctggggaggacaaggctctgagaggagacagagggcttttgttgaagcctgaggaaacagaacaccagagagggacaggggtcacaacaggaaagtcacactaaactgggattgataaaaagggaggaaaatcaattgatcatgttttccaagttaatcatcatttgtcattaccatttgaaaaaaaagaaaaatgatagaaatcagaactgcattaggatgacactccaaataaaaatataacaaggaaactaaatgctgcccttactcaccaatcagaagttgaaaaataaccaccagatacactcattaactcatccacaagcatttgcaatcaattttagtcaatggcatacaacaagcatcagacaagtctcagtcatcacagagcttatgctgtcatgaagaggaaaacacacacacaaagagatatagaatgtgaggtcaggtgttgacaagagccctggaaggaacagagcagggaaaggtcagaaagaaaagacccagggtctgtagagggggtgtcagggaagggatctcccaagaatgccctgatgtgagcaggacctgaggccagtggggagggagccatgcagacccctggggaagagcattccacacagggaaatgccaaggtcaaaggtgctgaaggaatgggggtgtcacactgctgactttgactcagtaggacacacacacacacacacacacacacacacacacacacacgctccaacgtggaggggtgaagagacctgctcaggacccagggccctgtttttccaccctaatgcataggtcccaatattgaccgatgctctctcctctctcctagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtataccgtgagtatttccacatgacctctgggtgttgggggtcagttctacttcccacatacgggattgtcaggcctgggttgtgcctgtggccctctctgcattacatcctgtatcagggtttggacatttagtgcaggacacacacgggggagacaaacttccacagatcagaattcctttcctggatccagaccctgcagacactcgctgcagaggaaggacagtctgatggggggactcagcagggcgaggtcagtgtcagacaagcaccccatggtctccccatggacctgaccctgagaaagaccctggagaactggatcagggcctggcctgaggggtccccctagggacccctcagagagaagctcaactcttccctgaactgagcccctgtacaaagccctgtccctggttcctgattccaggtgatcctggggagtctgtgccagggctgtgtggtggcctcttgggcagggctggctgggagcaaggatgtgagctatccaagggccatgactcctggagttggtcaccagccaggtccgagcacccagagcctcatctagctgaggatagggcctcctccttcacttgaggctcagcatggaaaggacagatg

SEQ ID NO: 125 - DNA, 2001 bp, Homo sapiens
tgggccctttctggttcctttaaagcttcccctgaattgtcctggggtggtcctgaaatcaagccagagcccatttagtataatctcattgatccattcctggagtggagtctgtgagaatccgggtgacctccacacctcaaggccgccttgccaatgctgagatacctggatactggccagaaccaccagtcactcaggatcagggccaaatgggcagtttccacacctgccctggtagacttgaaaccatggggagccaagcagaggaaagaaaccactgacacagaagccacggaaatgtgctggagaggccacatggaggccaaggccccagggtaccacagagactgcctaacctgtcgggtgcttctggcagtcctggaacaatatggcctgagagtttagttcatactaagtgaataagaacaggctcaaagggcatgtgactgtgaccacactagccccagagagggcatgaccagctcttaaaaaccagcagaggaagcctcctgacccagtcacatggggaaccatgtggaggccgagatataaaggaagaggaggaaggagcatttcaaggaagaaacagtcccgcctaagagggtcaccctaaaaacaaggaagtaggcagtccagtcctgacctgctcggagttgaagcagtcggcgttggaagagcctgaacacccggcctaccacttgtccttccatctctgggccttcaggacagagcggactcccctctcccctaacacagacagactggtgacagtaagcctgagaaaggggctgcttcggtctccagccacaactctgtgaagccaagccacgcgctgtcttccagagagggaatagaagtttccatcctgtgcacccagtggttgagcaagaaggatgtgacgggacccaggaaggcagcagaagccagcaccaacagagtcagcatcctggcactgcagcgcagatctcagctcctcccaagagccccagggtgtggcgagagaaaaggtcatgagcaaacacctctggctgaaagtagccggaaaggcctggctacttatgggctaagggttttgaatctgggctgcagattgtgcagagagttaatttacaaccttagaacagtggacaatgcttgatagtgtcagactgtgggcactggaaaccggctggtctcaggatagggatcctatacctccaacacttggattgctgacctttcacttctcaacttggtcaacggaagtcaaaaaccccagcaaggccacctgctgaattctagaacttccaggacttcccgacctagagcacatggccaaacactttctgcagccaggatgtcactgccctcccctgattaagataaatagacttggcctaataattatccaaatagattaattcttccaacaaaagaaagagttctggaagtcaatacccaatagccttctttcctgatggtgcagtagagagcaaaatccatctctaagcccagagttcgtgatattttggaaatagctacaatggtgaatttcaccccctggagagttctgtcactggcacttgttccagggttccagcagagtccctcttctcaaataggaagcactggctcagaagactgtccccagcataaggtaggggactgattgcaccagcagcctgagggtgattccatggcagtggaaaacatctgggtgatctccacccaacccccacgcctgctcagccttcctccgggtcaggccaggtcagctgttctgcccaagctacaggcacagctgctagcccactgctgtgtctggagactaggtaagagagaaggaatagcaaaaaaatcaccttggattctcacagaaacccctgaatgagtaaatacagggaaggcttttaccaggaagctgcctcaaagggaaagcagggggcaaaagggtttgacgccagtgtttgccactcctgctaagaataagcacccctctggaagagagtggggtgctag

SEQ ID NO: 126 - DNA, 2001 bp, Homo sapiens
aattaaagaacagcaagtcagtgccacttaaacggtaatccaaccaccgccagtcagtgaacagctttttactgctccatgaacaggtaagtatagaaattgtgagtaagcatttagaaataaaaacaacaacaacaaccaataattgaaaaactaattttatactttttggcatactcattgtacacttgtaataaaaactgggcttgtagtttttgtctcttttcttatttgctagcaggttttgatttgtattttacagaaataatggtccatgtcagattggaaataataaaaacttgtcttttacccttgatagtttgagaaaaaagtaattcagtggggttagatatggcatccgagagatgattcgggaaaggtgggccagggccatgtttatgtggaccctccaatgcctcagagttcacacttgtttcagtgggcaacaggactagtaaggtttagagggactgttaaccgaaggtgagtggactttgcacattttatggtattttttttttcccagtaggcatgtgcttattctttcttttcttttctaattaaaagttctgtttggtttttttagttaagaggagagactggaactgctcagtgtttcacaaagataactgtttgtatttgataagagtttcgttaaacaaaggaatttgcacttatcaaaacataaagcattgaaaacactgagcacatactctgtgaccagaatgcgtgtgacttgttgagataagccacaccgggtaatcagatgagtctcctttcttcttgggcaccctcatataattcaggcacaaacgaaagagaaaacgctttttctgatctctcagttcgggtcatctctgccttccatcatcaccgcctaacaatcctacacctggcagggtgggaaaacggtttaataatctcttccctttgggaggcgtttgacagctcagaaagcatggcctttactcaggctcagagacacacaaaaacctttcacgttttcctttcagcttcgcagccccgtccttctccacctgcacgggcacccactgaagcacaggacccttctgccaagccatgagccttctcaggcccctgccgcccgcctcgcctgaggctgcgcttctgaaaagcacgcggccctggcctgcgcaccacccagtctgaaggcgacgcccctgcacgaaggagtcaggcctaggggctcgggacccagggcgacactcaattttctaacaggagccacagcccagaagctgagaagccttccggccgatctgtggccctgaggtcgcccttggagggcgagtgagaaccccccaggcgttccggaggtggcccccagcccgaacccggcgcccgccccggcttcccgagtcaggtctttgccccgcctctcggccccgcggcctggccggcaagcagggctgcagtcacggggcggcgcggagggccccagcccagtcaggggtgtggccgccgccaccgtaaggctaggccgcgagcttagtcctgggagccgcctccgtcgccgccgtcagagccgccctatcaggtgtgtttgcagccgaactggcagtgcccgcgggcggctgggccgaggccttggctcctacccgcgggggtcgggcgggctgaggctggagcaggccaggggcgggggcagagggggacggacttccagcatcgctcattcgggggcgtgacgggaggacctggctggggagcggcgggttgggagcctgtggctgccccttcggtgacagtggagtcaccgagcctagaaatggggaagctgtgacgtgggggtggtggagggtgctgcgggacagcagcgagcacggccctcagccgtccttcccaacacggtttcacttcccctcagctccagggctcagccccaagccgcccccttcatcccccatctgcaaacaatcctggtccccctgagcctccaatatgccttatgcagttcttttggggttcctttcgcatttt

SEQ ID NO: 127 - DNA, 2001 bp, Homo sapiens
ggccccaatcagcagtgccaaacttttcttggagatgtgctgccggtgaggctcttcatagggactctgccatcatgggtatgggggtatcaaacacacactaatgaataaacctctgctcagattcatagggcaggaacttcccccaccccacaatgaatcagaccagttcctgctccctcctcttacttccccctgtacttatctcagggagtcagcaaagggggtgggggcgtctgtgtctgtctccctcttctatgtgtctgtcatttacctgggtaactttggtgcctggcaccctttaagtgcttaataacagcgagtgcttaagaaaatgcctgctgaatggacaacctaactcataaaccccattggtgggaacctgcattcctcagtaatgtcggatcagtgtttctcaactcctccccaccctaccccatcacccgcccctaaggagctttgaaaagatcttttcttcctattctagaaccaattccagcttccacacctcccaccttgagaaggaatgcattagatgaaccaagacttctgcctatatacatgggctgctacagttggccaccccggcagtctgcggcctcccaagcgggggcctgggtcttaatccttacttctctctaagctgcatgaagggtgcccccattccatccctcttcacttctccccagcatagcgcctccataaatgtttgtgaactggatgaatgactgttgagcgcttggtatgatgagctgcttacaataggtgctcaaccaatctttgtccaatctatgaatgaatgaatgaacttcacccttttccttccatacctccgggctgagcctctctcccacatggaaaccctaaggctgagaccaccaccttcctaccagttcctgggggtgcccagtgaactgtctcagtatccccagtgcctagcacacagtaggcgctcaataaacgcttgctgtgttcataatgaatgactcaatcaggcccacggtgggggtgaaggggggctccatactgtgcctaaggatacagtgaatgagcaagtgacacacactcccccaccccaatccctaactctccttcctcccaggatcgggggagaaacctcggcccacagccgggatgcgcccccgccggggtcaccgcggcggcgaacgtcccggacttcgcatcccggtgaccccttgggatgaaccgaagccggacccgcaccttggccgtaacaagcgcgctccacgccgccacgcaccagcggtgactcccgagtcctctgtcgccctggcctccccgcaggcgggtttcgaggcccccgcagggagttcagggccacggccacgtgcagccgcccccgagcccgagtttgcaggctcggggtccggttcgcgctttggtggcgcccggcgaggctgcagcgcgcaaagttcccacctcaagttcccgtcgcggcgtccccgcctcggagttggcccggccgcggcttccgttccccccggctgcccacgggtccggggcgaggacgcgagaggggcctggaagtcgcccggggagcccgctctgcctcccggagccgccgccaaacttcggtcccggggcccggctcagctcccgccctcggagccacggccgccccgtctctgcctcccggtccccgccgggctccgagtgcgccgggagcgcggcgcagcagccgccgagggagggagcgaggcaggaggcgggacggagggaggggactgagcgcggggcggggagaaggggcgggcagccgaaagcgcagcgcctggcctcgcctcgcttcgggacagtggcagcggcgccaccgtcacggggcgtccaggcgcccaatcgggaccccgcaccttggatccgggggccctggcgcgcctgctggattgcgctcctcgcggccagttcctgagcccgcgtccgggactgctcgcgctacgccctccctcccgttgccttcttccccgtctgctctcccaggag

SEQ ID NO: 128 - DNA, 2001 bp, Homo sapiens
tttttaaataacttcctcctcctgccttcaactctcagaagattatcacaaaaacatccatattatgaatagttttataggtcagttcccttccgtaccactaggcatacactgatgatcatttccaaatttttccagactgctttgatatcatgtgattcattttaagtagaggcagttttgtgaattccctagccaaactttttagattgtggattttcatataaataaattgacagaccggtgggggtggagggggaaatagaaaaaggaaaaaaatgtttttgtaaactatcttactcctaatctgttattgagtgagccatttttggctctaaggcaccctagcccgttaattcaaggttaccttcggtcattgccatggctccagtgcttattatcattgcctcatccacatcttttgagaacactctgagaatctacacggctttaacacacttcttcgcactagtgagaacgcccgcaatgccttattcagtaaggttaaatgagtttgtttgctgaaacattcgccacatgatcccaacgctcctccagctccatcccgtctataatcgcagagcgaacactcactcaccttgggcgtgtcaataattgatggtctggggatggaagcgtgaaacaccactgcttcacagtccgccccatcatccagggacccgggcacatcttcaggcgtctccgcgggctgggccgcagtgcggtgacttgaattccgattcgcactgccatttctctctgcatgggtgctcgaatcttgccttttctctggagctccttctgcgcttgggtgtcttggctgtctgcctgctttggccacaggctgcacacggacggtggaatgagtatcagccacgccggactgattgaaggaagcactctccctcgtattcccaaaacaggcgagcagagctgagcacggaaatggatgcatgcttctagagggagacttcagtcttttggtgagtcttgctttccacgtggacatgacttgcagtgaatcattgcaaagttcaaacggggcaaggaaattttccaggggtccacatcacctccactttgcttataggaactctttccaattgcccgaagagaagaacttgctagtggttgcccctaaatagaagttgggatggcaaggccaccttccctgagagtcacttagccgctggggcccctttgaaagtgtccatcctgggcaatttcgcgctcttccttctgtgatgctgtcagtggctaaagaaagctgttggggaggcatgtgagtggctccggttacagttcctaggcagggcagacgcttcagggtagggtagtgccaagtcaatagacaattgcttcaggttacctaggaggcataaagataatgtagcctgatagcatctgccataatagcgtcagcctgcgtatcaataacatcataacagcagcaaaaccttctttggttaccccccttcaaatatttattcccccaaacaaaacaaaaacgacccagttgtccatcacaaatgaagacccctagaagggccacgaccctagcagctgtttcattattaaaataggaggtagactcaatctttcgcctggagagatgcggtgtgatgtgttttctgaatctttctggacttttcgggatctttccggaatcttcccggaatcttccccccaccctcaaaacaagtttcctggtttcaggatccacattgccttttctgataactataaataggtctttggtaatttgatttttttttttttactagctaggctaagttcttgattagttttcaacaataccaaaaagaaaaaagagagccctacattaacaattatccttatgtattatgttattatgtgtggaggtattatgtacatgtgaagtattctacctatatggtatatatcacctactttcagaaagtaactcctttttgaacatagatcttctgtttctcaacacattaatatgtgaatggtgaa

SEQ ID NO: 129 - DNA, 2001 bp, Homo sapiens
aagtccatgacaagcaacatgccgtcagcgtaaatacagcgcgggtcctctagcacaacagaacccctgacatccactccattccactactttggggagtgatgaagaaagggagagggttggggtcgtcagcggcggatccacccctccatcaacccccgcccccgccccgctccaggctccctggcccgacaccgcccacaaactaaccacttgttttgtccacgccccctcccatgggcacgccccaggccttggccccgcccacctactccggctggacttgtgcccggccctgccccttgcgaccggccctcgcgccggcctcacgttcaggctccgcccccggccgcgcctccctccttccctttccgacagcgcgggaacggctgcggcttcgggcggccgggtttctccggtctcccaacgccgaggagagcttgtaacaggcgctggagctggcccgccaccgccgcgtcagggacggcgctggagtcctccgttcccctcagcctctgagctgaggccccacaccagagtagggggcggcgcggcacccgtgccccggcatggcgacaccggacgcggggctccctggggctgagggcgtggagccggcgccctgggcgcagctggaggcccccgcccgcctcctgctgcaggcgctgcaggcggggcctgagggggcgcggcgcggcctgggggtgctccgggcgctgggcagccgcggctgggagcccttcgactggggtcgcttgctcgaggccctgtgccgggaggagccggtcgtgcaggggcctgacggccgtctggagctgtaagtcctcgcccgcggccccttagcaggtatgggaggcggggggctgtcgggggaacgacacacacagaggtgaaataagccccttcagcgacggcctgccgcgcaccctatgagcaggggcacccttcactgagcacttgctgtgtgcgggacatcgaggatccgggaactcgaggcacacgccttctgttcgctgtaaggtatagtgatgataagtggaggcaaatcacttttgatcgcgtacttccatctctaaaaaactggactcgtacgtccagtgtaagtatacttatgtataaagtgtacacatgtgttaaaatacgtacattttaaagtacaaaaatagatacttaaaacgtgatggtggaatacgtatccgctatcctggtggctgtatgctctgcttttcaaagcataacgttcactttaaggcaagattgctttctctaagaatggtaactgtgaatccatattttagtgttcttgataatttctggctcttggatgacattgaactttttttttaacctctctgtggctgacgaaggattcctactaggagcagaggtgtcggccctgccattttatttggttgtgtttctgttattgatattatctccaattttttggcaggcattcttttaagccacgtgcttatttgtgaggacaacttttgcttaaaaaaaaaagagtacagtatctacagatgcacttaactagcaaagttaactaaaacacacagaaaaatgagtgggtgaggtgccatgttcagctcctagtggggccagccttccactcctcttggatacccctcccccataatttatgctggaaaccatccagatgctaaatatttctaatgcttaatttcttatgttttagataaataggggaaaatgggagttccaatattttcttcctacactccactttgaatttctttcaggagatgggtcaggagaacttgtctgaatccccaaattggaagctatagaaatgtttgcccagctatctggcttattctggatagagtaaggagagggcagggccttaacctggggcccctggatagaagagtatttcagcacaattgatttcctttataatcctagatattttatcaaatgcatttaacactcattctgatcacaggtctctggcactaaaacaggttaaaattt

SEQ ID NO: 130 - DNA, 2001 bp, Homo sapiens
atggaggcaggttggtgcctctggcttctgaagaccttacctctcagtcacaccaggtaagatgaagaacatccagaagtgaattccgaacacaagaatgacctggaagatgacctttcccagtacagtcttcctgaggtagagggctcggtccaccaccatggttccaaactgaatgaggaccatcaccaaaaacggccccgggacctggtcctctgacagtgaagaggtgatgtctgcagctgctgagtgtttctggggaagagacaacaaattcatgcccaagagaggcaattcatggcaggaagcgccatcgccatgggtcagggcgggtgctgtggcctttgccccaacctaccccaaaggcccaaaagccgaagacaatgatgatgaagtccacagtgtcagccaggaacatgagtacatacacgtcagtcacggcgctatactccgggtggatgaggttgtaaaagaactgtttgatgggcacatagatctccagcgtcctgcaaaatggagacccccacccccaacccacttgtttcaggaagggcaggatggcagaaatgaccagacactgccatcatgccactcctctgcattcctctttccagtcaggtcccaccttcctctctctgcctcaggatagtcccgctgtggacagctcctgttctaacggagagcgtatacacatttactgccaagataggtactgcagccctctctgctgatggcaccacagccctctcctcttgcttgaagcataatagaatggtatgatgccctgggaacgaggtgttgggtttgtagcctgattcaattgctcaccaccggcgagaaccatgagcatgctatgtaacctctgtgtttagttttctcatctgtaaaactggagtgatgagaagacctgatgcctagggccaaggtgggaagtaaatgagggaatgcatgagggcgctcagacttggagtgcttggtccatgcgcgtttgaatgtgcaacgcccctgctcctcctgttttagggcctgtgtccatgctggctcccacatactcccctggccttttatctatgctttgctctgtgccttagtgtggtgtcctgttggctcttgcttgtctgtctaacctttgccctatctttgctctgggacctgccttcagttcaacgccacgttcaggtgccactggctctcctgctctcagagcctctgtccagggctccactcccagctggcagtcccagtcccatgcaccacttccatccccagcaggtggagggacctgtgtagtgtgctgggtaacaggtgtatgaccatggacaaagcatttccccaccatgcacttccatccccttctcaggataagagtgcaggtctaaggcccttccagcattgacactgtattcctctcagtctggctggacttcaccatgataatgcctaagaatacaatccatgagcccttgaaatgacccatttactctgttcaaatagaaaaagcatgtgcataaatgcaatggatataaaatgaatcacacttacagaaaaatgatttttaaaaacaagacaaaaaggggtaatttcattctgccttacgcagataacatttgtgacacgagaagttacttctctgctctgctcctggtttataggaaataataaagcccaaaaccctacaataaagcacacatgccatatgttctcatggactcacttctttatggtaaaggcttttgctttgattaaatgttcttgaagcttttccatataaagttcccttttgcctttttgcttaatactcaaaacactgcttcctttttgactgctgtttcgggtgcttgtgctgcctagaaataaaaagagatcataagatgggtggtggagcctggggtttgttcacctaaatatccaagaagcagaacccagagcccacatggtggagggatagctcaggactgtttgggaggatgagtatgcacggccttgggatttgtcctcaac

SEQ ID NO: 131 - DNA, 2001 bp, Homo sapiens
gtaaactgacactcggtctctgtgtgtggtatccaggcagggtttgatggaaaaagcctgcttccactcaacctcaggtcactgtctggggccagccccggcccacccccctcccacatacccctcccctccccacccgccctctctgcatctacagccccaccagcacactcagccaggagcagtcccccggcactccaggggcacacagggctcctgccgccccagggggcctgcctaggctgtgcggctgtgaaggctctttgcctggtgtcttcctcctgattactgcacaacctccctttctcctgcctcctgcaactccccaggctccccagcaggcacacccgtgcacctcggtcctttcagcctagacgcagcggggtgcccagctctacctcctgtttggtccgctggtctgcagcaccttctttctgggcctcccccttcagcccttgggaggccccgccagctccggcctcctgctctcctcctccctctcccagctcctatccctggtcggactacatttccctgcacgtgtaatccaataactaactgtcgggtctcattggaagtgattgctcccgagggcacatctctctcagagcctgactcttacttagggaggaattagccacaactggggagaagtgttctcggacaggagttagctgagacctggcacagttttgggtgggcaatgttaagacggaggatgtaaacctagtgacagctgcacaccccttcaggactgactataaatatagccatctcgcttatctcagggtctttctttttctttctctttctttcttttcttccttccttcctttttcttttgcacttttttcttacagaacactaactggggtgcgggggtggggggaagaagtggaagcaaatgcaaccctaagtcccatgagatccaggtttccagagtctttgaaacttggcctaggtttctcagtgtaaatccctctgctcaaagttccgaaaacgtccacgatcgccagcggcccgcaaccctgcggcggcccccagtcgccctaacaattgtcggggagcctgcaggccctgccagaaagagagaggattggcacgttacctttgggcgtctggcaccccggcgtgtggctgagggcggcaaagtctaaacgaagttgacagatggagtcagtatcggccctggggagggcggccagggaggctccgagcagccgcacccgcagcagcaccactcggcccggcgggccaggaggaggaagggggcgcggaggggcggggctgggtcctgggatgggacgcggggcttgctgcggggggcaaacagaaagaatatttcatgaagtcagaggtggtcgagttttgcgaggggtgggggtcgtccccctggcagaaccggagtgagtctgtgatccctcaattggaaagagggtgagtgagggaaaggggaggaggccgcgagaggaatgaagagcgaggagaccctggagagcggggagcgagataagagagggaggcggccgccaggtcgggagggggaggagtgagcgcggcggcgcggagcgaggagggggcagggcggggtggggggggagggagggagggagggagcgaggagggcggccggggagggcgagggagggaggaagtccgagagacagagcgaggagcgctcctgaagggagacgtcgggctcgtccccggggtccaggccgcggtcccactcgcgggcgcagtcgcccggcgccggccgctcggccccgcggcctgggaggccgttgcgcaaggcagggcccgcggctcggggagggatgcgccgggcgttgcctcccgcccgccgccgccgccgccgccagaagccccagaggagctgagggaggcgacgccgaagcgctggcccggagtggccacggctgcagcccgggcggcgggctagggcgctcgcagggcttctgggccgaccccgctccggcgcctccgcttcccgcccggggc

SEQ ID NO: 132 - DNA, 2001 bp, Homo sapiens
ttctccctggcccagtctagaagcttcttgcaggcaggtaccacagtttggtcaaggtgccatgcgtcccaggtcctagccaggccctgggctgccgctggtgaacaggaagggggtctgtgccctgccatgcccccaggtagagctcccctgggagcatcctccctggcctgggacctcccagaccccaccccccggttgagtgatggcactgccaggggttgaagaccctcagccctcgacgttgtcctctctccattggatgccgcctctctctagccacccctctctccctctctgccccttcgagctttttctctcaatatgcaattttctcttttggtcttccgcactcttggcccccagttctattgcagatctgtttctcactccatctaaactcttacccctgtgtctcaggagctgctcttgctgagggaagaaggggacactacgggacaggggggcagtgtcgtactaaggacctgggctctagccactggaggaactggactcatttgggccctcaggaagcggctgagtcttggtggggtaacccggttagcccccgtaagtgaccagcacagggctgagcccagaggaagtggccacccacagagtggttctcatgtccgaggggacctgcagggattgagcaagaagactgactcgctggatccttcgtctctgaatcagttcagggcaggcaagctggggagccccctgccccgtcctgccaccaccagccggatcgggccctctttttaagggaagaaagtctggagtggaagggagggcacagggcgccaggagcctacatgaagtccttccagaaatccacaacagctacctctctgatcctggagaaaccacctccttgcttaggcccaagcaggttcctggcaggctcaggaccaaattccaggggccactcatgggcctagcagcccaaggccgcctccccctcgtctttcttccatctctctttcctctgcctggcgagatgccagccagcacctcagtgtccccatctgggcagtggaaagtttgactctctgggtccttgtttgagtgagtgcgagtgtgtccgttcctttgctgtctgccccaggcgggggagggggggggaggtggtgggggcgagggggcgggggctcagctagtccagccgtctacaagaaaattgctccctttgaagctgccaggggggccgggaagcctgccccctcctgctcgcccgccctctccgccccaccagccccctccctcctttcctccctccccgccctctccccgctgtcccctccccgtcggcccgcctgcccagcctttagcctcccgcccgccgcctctgtctccctctctccacaaactgcccaggagtgagtagctgctttcggtccgccggacacaccggacagatagacgtgcggacggcccaccaccccagcccgccaactagtcagcctgcgcctggcgcctcccctctccaggtagggctggcttcaagctgcctcctcagcaacccagagatgcccctggctctgctgcctccgctgtcccaagccctggtcctgctgtccccagtgccgcgagggtgtccacagatttccccggtgctctctgtaggctgctgatccacgccccttcatcgccaccctgcggcccccttggtccctgtcaggcttctgcctcgtctcgccgccctccaggcacctttccctcaccccttcctctccctctcccttctgaccttgctctgcttcatccacctcttgtctctctgcctcccgctcggggtccgtcttcttggctaccaccctagagcgtggctgggtgactggtaccccagctttgccaatggccctgtttcatcattgcaagtcccaggcgcatgctccactccctcagcctcgctctgcccaggcgcctccttgctccagggcttggcgcctggcccgggttgggtcggatcggggaggaccgcccagcgcccaccga

SEQ ID NO: 133 - DNA, 2001 bp, Homo sapiens
ctgggactcctccctctgttgaccaagtgtggaaccctgtaataacttcttagcaaccctcagaccaaggtcctcacctccctgagacccggcaggcagaagtggggaggggcaccacacgatcacacctgcatgggattcccaaagctgacagaaggagcagacatttggcgggggggggggggggggtggtgggcaggggagggggacaagacggggacttcctttatttgggtgtcctccaggtattcagatttactcccgacagggcccagccctttccctcctgctgaatcgtgtatgggtcttgcacgccaaagccaccatctccctgggatccccggtgagaaagtgggggccacttctcttcctcaccgtcctgcctggggttccagagctgataagagggacagatttcctagttctgagatgcggggatctgctgagtcctccctcagggctctgggactcctccctctgctgacctgaggctccactcctcagaccatagccctctcctcattgacaccccaaaggcacagcaaagacacatcacatgggccaccgtgcctgaaaccacccagggctgaggtccccactgatctggactccaaggtcccttgagtcctttctcttcttcctccccttgactcttggctgggttgttctcctctaccacgccccgccgcagacctgagtcaccgtcccttggattcctcaggccgagtgaagagacatctcggtctgagacagaggtggggtgggcaccctgtcctgggatcccttcatgcctccctcatttcccagcagggcccgggccttccctctgctcccctgaagccgcattcttggtaccaatatccccttccctacgtcagcctcagatgccgacgtcagaatttgcgttcctggtcgccatgaccagggcttcccagggccgacatcaggggcagagccctccctgagcgtcccgacattgatgctggcagaacctaggcgcggctcccgccgaaccagccgggccctggtcacaccaagctctgactcccagagtcccctgcggcttaagcggcgggagagagtgggggcgggggtggggggggggtgacagcccagcctgagaagtctgccctcaggtggtccagagctggcagcagaggcagcgctgtattatttagggctttctatctgggttgggggcgtcctcagtcctccctcagcctctcaccttgcctcctcacagagcctgggcccgctctcttcagccgatctcaagcctgtccctcagaacgagaacctcgcttctctctgatccccgaggcgtaagttagtggcgtcacatccggtccccggggcttccctgggttgacagcaggggcggagtggattctgccggaactggggtgggagttggggtgggagtgagggtgggagtccgggtggggtggggtacgggtgaggatggggttggggcaggggtggtggtgaagatgagtgtggggaggggcgtggaggtgggaactgggatggggatcttggggctgtgggtcgtggtgggggtgggggtggggtctttcagtcatccctcagtgtctcgaccttgatgcctggaaaagcctggacccctgttttgtgctgaggatgtcttctccccttaaaccaggtctcttgactcggagtcttccaaggtggcagtgaggggagggttgtggtaggggtgacaacattgccagggtcttccagggctgacaagaggggctcagctggattctgtggcccctttgtgaggggtgggtggaccctcggtcctcactcaggagggtcttcctcctggcttttgtctctttgatgaagtgatgggtgggagatgctcagtttccgtcacctctgatcatttgcccagctgcatcccttgcagagcatgggtgcggttcccaggccagatggtccctggggagctgcaacacccgtgattgtaatgacctatgggagaagacgcac

SEQ ID NO: 134 - DNA, 2001 bp, Homo sapiens
tttaacttctgtatggtgagatcatggatattaataatacttttccatattttccaatatttctataatgaccatttatttctcttataatcagtaacaaaagaagggaaaacttggtctaaacacgaatttagggacttaaactagacttggagaaaagctttctatgcaagatttattagatactggaaaggatgaggaagacaaatttaagtcaatatgatggaaagagtggtggatttggaatgaggaaacttagaaatgaatctgttttctgggtgaccttgcgcaagcctttgctaaaatttggttttctattttgaaaaacgcagctggctgtttaaggaagataatatccaccagggcgcctggcccagaaaaggcgctctgtaagtggtaggtggatcctgtctaatcaacgccaattataaccaggcatctgcaagctttgtggagggcgcatctttcactgctaccagagcctacagttctaataatgaaaattgcctggaaaagaaccacaactcttaaaaaaaaaaaaaaagtccaaaatcacaaagtggcttttgcagcggtccttgcctgtgacgccgggccatggctggagctccttgctccgtacctctcaggagcctccatcctgtaccaatggccagagagccggggggtgacagacaggggggccggcagccgagccaggcaggacgctgcgcgccgcgttactggagccggtgtcccggcaaccccggaacgcgcctctgtgggagaagcaggcggctgcggtgtcccttttgccctaggctctcggttctcaggcctcggcgtgcggcgtgcgttcggcagttcagttgccagttggttcgttggtccgtggaggcctctgagcacagacccgggcctcccctggggaagggggtgggaagaccccgaggtggaaaagaaggagcaccttgagctggatgggggctgcagcgagcctggttcgggctgggggcggactcgctggggctcccagggcttccaaaccacgaaacgcccaggtggagggagtaaggctcccgtttagaggccagtcctgccctcctgactttcggtggcttgtggaatttgtttgttttgactgatacctttcttacgagggactttctttgtctctgggttgcttctaatatttaaaatcatcatgggcacagatgagtccatccatgctcactggaaacagtccacggggctgccgtctcccctccatctcaagcacgacctgcggcagacagctggagaaggttccagaaaaaagggactctggcatgaccgaggtggagaggacctacagttgagttctaagaaatccaggctgtggagttggtggccacatccaagtatactacacccgggggcgggggcaggtggggggcaccatttgaagtttagaaggaagctagtttgggggcatgtaaaaagagctgcttgaatagtagcttatgtttgtgctaacccaagatttgattaggcagagagcaccaccaaagcagagagtctcataaattacctcactggatctcacagcagtggagaagtatattacctatgagtaaagggagccagagatttgacctgcccagtgcttgggttaataaaaggtgagcctgggatttgacatgagcctgggatttgacatgagtctgccatactttcgagcctgtgtcctttcatctttcctttgctcagatattagtaggtttgtgtcattgtttattggatgggacagttcaggtgagaattatgaacactctaaaaattaaataatggctggtagctccataattggttaaggccgaggagggtatttgggggcacgtttgcctttggagaacagtgtcatggcgctgaacagccacagtctcccagaaaagctcagagtgcgcctgcgctcaagttctcatttacctgttatgggtgagtggcctgagtgctgcaaaaaggggttgtgaaatatccaga

SEQ ID NO: 135 - DNA, 2001 bp, Homo sapiens
tataggacgaaaaaaaaaaaaagtctagcaatcaagggagtgcgggagtacggatgcgtgtgtgtgtgtgtgagtgcgcgtttaaagaacataaaacgccacaaataagcacttaatattttactgagtcgtcatacagtaactcatttctaatgagactgttaaagctgttaccaaattccaacaggtcatgaaaaaactgctcagcacctctctgtcttctgtcaggttagggctccgcagacggacgacagaggattatccgtagacttagatgggggttcctccgcccctctctgtcctcctgccaccacagccaggctggacgcagaaccgcacggaagcctccccaactcctgcctctgggagtgctcctgcctgcttcaaggaatctgggcagcgggaccctctgggtttagcgtcctcgcctggggaagcaactttggccttaccgtagaagttacaaaattggcgggggtagggagaaagattataatatatatttggagtcctttcttccagtttaataaacacgtacttttctcattgccctagtcctggtatgcctaatggcggggaaagcctcaaataaactcggtgccctctggatctccccaacctgcctttagtccttgcgagaggcgtgagagaacttcctattatcatcccttttgttatttttattacattatttaataggctccctctacggaaccaaaaatagccttaatatgcttactttctccctgtactccgccccaaactgttagaacagcagaaaatttttttttgggggggggcggtttgggggaaggcagaaggagaagaaaatttgattgtgatggtggtggattttttccaaatttccccacagctgttatcaaaacatgcaaatgagattttccaacaggatcttaaacaaatctggaggagttaatgccgaagcaaaataatcagcacgtttgaagtgactgtgagtttcagtttgtcttttctccccgtgatattattgcaggggaatgataattagacctttagaaatccaaaggggctctcttgctctcttccctctctcatttctcttctctctttctctctctctctctctcccttcctccctccctccctccctctctccctccctctctcttgttccctccctctccctttctctctaactctgatgttggcaaagggggttttcttaatcagactgttttttggtcccagggaaaggaggaagaaggagattgtgatggagaaagggggtctgtgaaacgtcaggcacagcacaaaggctttgccacgtaattacaggctcctattaagtcgagatctgccctcccaggggtctccaattttcttgtattccctacaaagcctcctctgcatgccagtttgtgccttttgaagtgccagagagcttcttgatccaactgagaaggaaaaaggagcccagcaagaagagggggagagagagaaggggaaaggggggaacccaccagcaccctccgtcggactcttgaagccttttttttttaattcttaatttttttttttactctttacaaaaagtaaagtgagaatcctgctctctaatacatctgcaagacatcaccctctcctcctgaaactttagtcactcctgagaatccacaggagtgcagagaggggggaacacgttttcttgaagatgttttaaagctggaacaagccttcttctgttggtgcttgaactcttgcctgggaataacttttttaacctttaaaaaaaccattcactttgattcttctctcccaccccttcttctctcttcttctgtttgcctaactcccccgccctgctggcctccgctttcctctctcccccttgttattatttttagtctgtgcgtgtggacacttttggagagttggaagggatttttttctcctgacttgaacatagggtgactttttaatattgtattttactgtggattatctctttggaccgcgccg

SEQ ID NO: 136 - DNA, 2001 bp, Homo sapiens
atatctagagtgatattttacctgtttctttttcttcatctggattcttcatgggacaccttgatatcactctagatatcaagagtgaggagaattctcaataaactggcttaataggattctttgctaaagcagggttcaggaggtcaatgtcaaggcttagttcagaagaggagcctgactaaagttgggtcaaagaggaagtctttgtctttgttaatctttcaaatttgaattcacccactaataaggaccaatttagaatgctatatgtactttacaagttcttttctagacctggaaaataagctccatcattactcataatgacattataggtaactgacgccagctgtcttcataagaacaaaattatctagcagaattgcacacacactgacaatttggtccaaatgaattctaggaaatctgattaaactcagagggtaatgaaaatatttgtagtatttatattgtgcttagtatttgccaggtactgtcctgaatcatttgcatggtttaatctcttagtcctcccaacagtctattgggaggtattactaacagtctgtagggaagtactattactagccccatcccaggattacagacagagaaactgagacacatagcagtttcaaagcttgtctgaagttactcaattataatatgtcaggtttgtgattggaacctagcactataacttttgagatgatgtgtttactacagtgcattcctacttctgtaaatctcttctccttcctttctctgagcaaacacataaaactgggaggtaggtaggagaattgggatgacattcatttgtggaagtccatatactgtgtcatattcttcatcttcagcacttttaatctctaaaacatagtgcagtatttagcaggtcatcaataatttttgttgttgggtaaatggctgatcgaatgaattaacaaaaggactggatactccatttctgatcaccatagagataaccaaattacgtttctcaaactctgtcatgctgagggctgcagtgacaacattttaggactagaacacattacttggatttcacaatacaaagatgattgcagttgtccctggtaagctcagcccacagagcctttcaattcagcttggggtgagctgcacttcaggcatggggatataatagtgtgatgaaccattcttctgaaggttcttctgaaaagctgctggtcttgggatactgtgagagcccctctcattgctctttagaagtaatttaaatttttctgctacattgtctgctcagctcgtattctggtcatagaggaacctgaagccagagaaactagacaaaaaggtaagagaaaaccaagcaacaaatgcagtctcagctgttgctaacgttattcacaggagtgacaaactgaatgaatcttagggtgagagactggtctttttacctcatttacacttttccttggtctgcactgtagatcagcagaactcatttatcctaagaaatcgatgactaattttctgtgaagcagaatcaagaaatctggtctacattggttaaaaataagtacttggttcaccttgtgctgtgaagtccaagccaaataattgttgcaagttgttgaagcatttgagtgaagaaaaactgaccaagttcactatgactgcatgaaatgaaatgctgaactttttggggttgagggggttggaaggatggatggaggaagacagttgtttccagtctctgaaattcaattctgcagtaagtttctgtatcaccaagtgtcactttggcatacctcgtgcatatttatagtctaaggcttttcatataggattcaaacatagattaaagtagcagggaacttgagagctccagggaagaatatgatactttaattgcatttgatcattttattgaatttgcctaggaaaatcaattaaaacacaaaacttaatttttgtacttacagcagagattatacattccatcttgtc

SEQ ID NO: 137 - DNA, 2001 bp, Homo sapiens
gaacgggaagacgcctttcactgacccgggcgcgggacctcgggtcccgggccggggccaggggccataccctggcgcggggggcgggccgcctagcgggggaggggggcacgcccccgaggtgggggcagcggctcgcccctcaggttaactcttcgctggccgaggccaggcgcgggcatgctccctcgcacccggccaggagaaaaagggcagcgggggatgggcgggcgcgccgggcgttgccaggctacggccccccagggagcgcgggggcgcgccgggccgccaggggccgtcaggggccgtgggccgcgcgcctccccgcccgcggggcccgccgctgccgctgccgcctccggctcctgcccatggccgccagccccgggagcccggccctcgcgctcggcgccgcggcccggcccgcgacaggggggcagcggcgcggcggcggcccccgcccgcggccaggccagcgcccgcgctcccgcctcgcccggccgcgctctcggagctcccggcgcccgctcttcgtccagcagccgctggccgtctccagctcccgcccgggacgacgggcacgcgagcgcgcccacggcggcgggggaggggcgcgggcacggggcgcacgcgcgccgggaggggcggggaggggagcggggccggctgcgggagggagcggcgggggcgctggagccggcgcgggaagccgggggcgggcgctcgacggaagcgggcgggggtcctaggcaccccacgacacccggcgtccggtcctgaggggcgggcaccaggcgcgttgtctgcgcgatgccgctctgggagagaggggtcctgccaggaggagcagtctcctgggtcccccagagagagtccgcgatttcagcccctctccccggtcttggacgcagcagccctcacgcctctgctccttcctcgggcttcgccttaaatgactgcatccctccttcaactcagccattccctgctgtctttgaaacggacacctttgtttaaaacaagaaagaaagagagaaagggaggaaagaaaagaaagagagaaagaaaaaaggatgagcgaagcgtggggagggggtgggtcgtggggagagtggcctcggaggccttgggacccgggcctgcacacagcactggccagcctgggcccgccgcctacctgttcctcagccccagccctcacccggtccccgctttcacagcccttccatctcttcctcttctctcccaccacctccccgtcttcttggacaagcctggagggagaaggcctttgcctggaaagcagcagcgggtggaagtctgcgttcccagaggctgccccggctgcaggagatcacttggagaaattgctacatgcttcatttccaggtttgggcaagtgagggaacccccagccacacggggctgttgaatcggagcccttcctttcctccctggctcctcgttagaagagaaatcaagaatggcgggcctgcccagaatgtggagaagagaggcgagtttcttatccagggcttctcccagaaagcagaacttgagacaagggctggtgtgcaggttgcaacctgccacccaatggctggctagtcttgaggaatggcgtcatattgagggctcagcatcggtctctgcagctggtggatggaagaacattcactagcggcagtgtctagatgagccgtggtaagaggaagcccttgccgttgggcttatacagagcctccagccctcccaccatggccactggcatcgtgggtccatcgcatcctggggagaagctggctgacctccaccagatggtcccctctgttcgctagcttctggcgcgccccctccacactgcatgctccccagcggcatgggcgcgaagacgagcacggggccccacactctccctgagcactcccgtaggtacctcttcgccaaacctgtctcttcttccaatcttcctccacccgggtccctgaccagtcagcccag

SEQ ID NO: 138 - DNA, 2001 bp, Homo sapiens
tcctattccttggaaagttccagtatctgagggaggaacagtgacttctggtcctgacctgagtggagaccgagggactcagaagagctggaatcagacccccacacacactgagtatgagtcagagaacaaggcctgagagaaaaagtcacagtgcccaagactgctgcaggggtcaaaggggactgctgatcagtgttccagggatttgctccagattaatctgagtcatggaaaaactgcctttcttttgtttctaaacaaatacctgcaaagacaaaagaccacataaatccccaggtgcacaactttttcagatttaaagaaaaaacaacccccgtctttccctccattcccaggagaagctcactctgtggcatcaagctgcctgggtgagctctcttctagaagagtccagggggacaggcaaggaatgggaggcaggaagtccagttcagggagggggattctgggatgaaaagtgaagggagagggactgagcccatgctgagggtttttctctggtttctcagacagctccggggccaagattcggggagacattgagacagagcgtttgatataaaagaagggggtaagagccaagtcccagggcccggagcttggctctctgggcttcaggccccgagggcggtgcctgggatgggccggctcagctttggggtttcccgagctccgctctctctcttccacctttcccaacctgtgtcaggtccttttacctggatactcatgatgcggcctcagttctcacttccattgggtgttgggttcctagatcagccaatcagtgtcgccgcggttcctggttctaaagtcctcgccggcccaccgggactcagattctccccagacgccaaggttgcgggtcatggagtcccgaaccctcctcctgctgttctcgggagccgtggccctgatccagacctgggcaggtgagtgcggggtcgggagggaaaaagcctctgcgggaaggagcgaggggcccgcccggaggagtaaatctgcacatatatttaattacagattacaattacaatcaaggcagaaatgatctcatttttacattacaactctggaaaaggcaatagactgagatgcaagtgtgcccccaagtgatgggcagaaggagagaaggtgttttggatgcattctagaacacaggtaatctaaggagagttgatcaaggccgtggaagagtcctccagccactattggccatcaaaggagtcctctgtgtcccaggaatggtcctgctttggtgtccctggtgtgacccatcacccgctgggaacagcctgagagaagtagggcctctgcaccaatgctgctgaggatgtcagagcacaggaacgaggccttgggaaattacctggaaatgcgactgaaatcttccttcctgaggggtctgggctcttggaaatcaaaccctctcaggttgggtggctggacgattctcctcacacttacaatgggacaaggggaaccaggaggcccccaaggggatccctgggttccacacgaactcctcctaccctcattgtgtgacagcagccatgcctcctcctggggatcaggatctattacctgtgcctggagaggaggggactcctcttctcacccgctggtctctggacacatactgtccaattcccctgtggcagctgtaatgtgtagttcaatgggcactcatttgtccccttttaagggtaccctcctttagaatccaggaccttctaccctgcagagtgtggttttgggagagaagtgcaaaatcccacgacaggtgagttgaaggaatgggatatggagccacatccacttccaccccttggtatctggacccacgtgttcttcctactgagattacagaactgtagagatgtctttgatttttaaaatgcaccatgtcctgaaagatggcaccctcccacccgcagagtgcttcctgcaagctggcgttgagctgtgcctatagaa

SEQ ID NO: 139 - DNA, 2001 bp, Homo sapiens
tctctgcctcagctttctccactgttaattgggcataatggacacaacaatggtgaggggctcttgcaggaatgaagactaggatgaggatgaggatgagggatacgtccataaagcccctgcacaccttccatttcctgagcactcccaggaagtctttgctgaccacgccaaattagattatctttgcttagccattaggccgctgccctatattgggaggcaaatatacctctcagcttcggtggccaggtcccctggcacccacttagtcctcagtctgcacctaaaagcaagccttccgagggtcttgccttgcaggtgtagctcccaccccccacgctggggtggtccggggaccctctgtttcctcagtggtggggaagggacccgtgggttcgtgggtgctgaccgacgaatgtgcggaggagggcgcgccccagcatggggggcgggggtgcttcttaggatggcttggaggggggcgcctgcagggctggacggcggacgggggacactgcactttctgatgcggtcggggaagccgggagtcagcctactggagtccgctgccatccctggccagaagcagccagcgccaccccaagctccccagcccctcgcccgtgccggggccgccggggtccgagttaggggagagtggggggcggccaccgctgcaccgcgcggccttcgaaggactgccctgcggcgccgcgttcccccgaggccaccccctcccgccgtcaacactccccggggcgggggctcacctgtgtggtctgggtcgcctccgaggccgagtccctcgttgccaagcccagcaggcctcgctcccccgtgactgcaccgccggcccgctccagcttcccgagtaccccacctgtctccccggcttccttccggccctttgtgttttcttctcttgctccgccccctcgcctgcccgctaagcacttaagctggtgaagtcaccctactgccacctggcagtcactttcgctgttggtggcaacccaggctggaacccagctcttgccggaacccctgccccatccttgaggcttgtcccgacaccgagtaggttctagttcctttgtgccttccaaatgactcccttcctccagcaatcacagccgcaacagccatgtattgcgcccccgccctaggccaggcgtgggcatcacctttgcactcctttctgcagccgtgtgtggtgcccactgtgacctgcacttcacagacgggtcatctgagttgtgcaggccactaagggagcctggagtcaaccccaggcctgcttggctcccaggccttctctttccattgctctttgtcggtcccctgctgcctcctgtccctccccaactctgacttgtggagggaagcctttgacatgtgggggtacctttaataccgatatcacttgttaccggagaggggtcccgatccagaccccaagcgagggttcttggacttctcgcaagaaagaattcggggtgagtccgtagagtgagagcaagtttattaaggaagtatagaaaacaggccgggtgcggtggctcacgcccataatcccaacactttgggaggcaaaggcaggcagatcacttgagctcaggagttcagaccagcctggccaacatggtgaaacactctctctactaaaaatacgaaaagttagccaggcatggtgctctatgcctgtaatcccagctactcgggaggctgaggcaggagaatcgcttgaactccggaggcagaggttgcagtgagccgagattgcgccactgcactccagcttgggcgacagagccagactctgtctcaaaaaaaaaaaaaacaaaaaaaaaacaaaaaacaagaagaacaacaacaaaaaagaatggctactccactggcagagcagctgtgtgggctgcttagctaattatactggtagttatttcttgattctatgctaaacaaggggtggattattcatgaattttccaggaaaag

SEQ ID NO: 140 - DNA, 2001 bp, Homo sapiens
atcgggttcccttagagctaagaataagggacagccagactctgtggcatcccttctttctgggccgggggtatccccagtcctcatgaagggggcacaccttgggtccggcagatcctgggattcctccctctgctgacctgaagtgccactcctcagaccacagcccgcaaatctgtcaccctgaagtcgcaatgagcatggggcacattaggccactgtgcccaggatcccccagcccaaggttgccactgatctggactccaaggtcccctcagtcctccctcttcttcctccccttgactcctcgcagggctgggctccaactccctgctgaagtgggtctgtatccctaggattcccgcggccgaatgaaaaggtggctcagtttgagacaggggtactttgggcccctgtcctggcatgctgggagccctccggactccattatcttccaggaggggcttgggcccatcctctgcttcccaaaggccgtactcacgataccgacccccttccctccctgagccacagatgtgaacgtcaggatccgcgtgcttggccgccattcccggagcttccgtgggttgattgcaggggcaaagctccgattctgccaggatgggtggggatgggaatcggggtttgggggtcccgttgatgtttgagcgatggtggtgggtttggtgttgggggatgggggtggggttggtgttgggggatgggaatggtggtgggggtgcgggggggttaccctcagtcccctcttggggtcctgaccttgattcctggctgagtctgggccctgagcacttctaaccagccctgccctggtcacacaaagctcttacaccagagttccctgcggttaaagcggcggggggtggcggggtgaggtggtggcgactgagcctgagaagtcttcccctgcagggtggtccaggcctggcagcagaggcagcgctggattattgggggccctctgtctggggtgaggcccgcctcagtcctccctcggcatcttaccttgcctcctcacggagcctgcgcctgcttccctccaccgacgtcaggccgttgctcagggcgagaatctcgctgttctctgacctccaacgcgcaagtcagtggcgtcacatccggccccgtgacttccctgggttcacagcaggggtggaactggattcttcctggatggggatccagatggaggtggaggtaagcatgaagatggtgttgggggtaggggtagggatagggacggggatcctgaggctgtagatcctggtggggttgggggagttgggggtgggaggggcctttggtcatccctcaaggtcctgacttggatgccaggctgagcctggaccgccatcctctgccgattgtgtctgctcccctcagaccaagtctctgactctgagtccccctgaagtggcactgggggaggggtgcggtcggggtgacattattgcctaagtttatcagggctgacaagagggactgagctggattctgtggtcccactacgtgggggagggtggaccctcagtcctcactcaagagggtcctcttcctcactcctggctgttggatgaagtgattggcaggagatactctgtttctgtaacgtctgtgcatttgcacagctgcaccccttgtagagaatggctgcgggtcccaggccaggagctccctgcagggcgggagctcccacgatcgtattgacctctggaagaagacagacactttcccacgggagctcctctccagccagagctacacttggcaaacctttggtcctaaatgatttattcactgaattgaagaaatacggtttacatatcttccaagtatatatgtaggtagcgatatatatatttatttctacgaactaacacctacccttagtatgcatcgttcactcttactatcctaatctacttctattccagtctttgtgttctgttgaaggaatttttttttaaacgtaattcctct

SEQ ID NO: 141 - DNA, 2001 bp, Homo sapiens
tgacactgactttttaaaaaataaacttacccttttaatcaaatgtgtgaattgtttaaaggaaaaacattgtgtaaataatgttttcagggggtagaatccagacgtgagttttggaaacagagtccagtcctgcttctgacatttgaattgcttctccagcatcatcaccagaagatgggctacctgtgctcacaagcctcctgtaacacggagctcaccacctctcaaaggcaccttctgtcaggtctgcctggaaatgctgctcccgcagactccgcacctgaggccaggtctgcagcgaagctcatccacacctgaggccaggtctgcagcgaagctcagcccccttccttcacaggagaaatacttctgggtgtgcaccttccactcctggcagggccgtgagcccctgtggcagtgcctcaccctctgggagggaccctctccatctgggaacaatgtgggtacttttttctcttaaagggcagtgtcaggaatgaaagcagatacccaaaagcagtgggtctcgtggcctcgtgtctgcttccagaagtggagggcggcctgggacggcgggagccctgaggacaccaccgggggggtcccgggacctgatacgagtgggcaggagccccaagccctggaggaaggaaaaccatttcataaataactgcatggggttgtgggccttttctcacaagccgcagcacctccttcgtctctccacggtgaacccgcccgccacgctatgccaccaggccaggtcctgcagcaccgcctccgtggctaggacttcctcaccagcctggggccgtgaggtggtcatttccccccttacctgacttatctcaggattcacacacacacacttgactttcggaaacttcttttaaattaatttcactgacactaaaagggaagctttgtggagtgtgattgtggttacctgttaatacttggcaacaattatgagccctgatatgagccaacttcttccgtcttatcccctttgaggacgctctgttatcgcccccattttgcaaacgaggaaactgaggctcggaggcctggagcaatgtgttccaggacagacggcaggaagccggggtaggaattccaacagggagttccctctgtggaagggaacgtggctggcacctccctgccttccaccctcagagctagtgtgaaaaagaagacggggttatggaaaaggcaacgggctggagattccccggattcccaggctagggcgccggtctcactgtgcgcgcaggtggggcgcgtgccagaacagggcctgggggtcaggccgagaccggcgggcaccttcaccagggccgcccgtggcgttcccccagcccctctgccgcgggccccacgtggccccgaccctgcggcagcctttcggaagggcggagctgaaaaacggccgcttggctccactcggccccacagcgcccaggagcgaatcccgagtccgttcggaaccgcctcgccgcgtggcctggggcggcgtcgccaccgcgcgttctcggcctcccggctgcggcagcgatgccggtccttggagctggagcggggagtccaggaggcggcagcgctccggagagagacgccctggtcccccgccgcgcgttcccgcctctgtgccctgggggggaggagggggcggtcgccgctgccattggccgtggggggtgaggcgggccccgcgcaggaaaccccgcccctccctctgcggcgggggcggccccagcggatgaatgaagcggcgcgtggctgccggggaggccagagcgtggagcgctgcgcggcgcggcggccgggccctcgagacggggacggtgagtgcgcggggagcggggagctggcgcgggtgcacagggcccgacctgctggcgtaggagcgcggcaggtgccgccatcgtgggcaggggctggagcgccggggggatgcgcgcgggggcgggaggcctgggcaccggccgagcgccc

SEQ ID NO: 142 - DNA, 2001 bp, Homo sapiens
agaatcactgaatgatccagctcacggtcatcacaaacgagcagaggtaacagggcacaaactcttctttgaactaaaaaatccaaccaaagttagttctgttgtcattcctgagaaccacggaaaggatcctggctgggctgtatgggcctccgcgcatgtgggagaggtcaagctcctccacccttcaacgccaatgtcagctatgtgtccttatgccaccaagaagcccccttcctcttccggggcctgaggttttcccaactgatggcgaagctattcctatatgtgtcatctctgtcctactagaacataactggcctgtattttagacaacttggtaatcccagtgcccagcacagggtttggtacagaataagtgctaatgtttgatgaatgaatgaatgaatgaatggagtagattaacaccaggaaccatgaggctcctgagcagcacctgatgaaagatcccccacactgaaaacagaatccagaatcgttgtctcacttgggcagtattattcccatcaattttttcaacaaatatggcctggctgtctactacaagccgggccctgtgaaaagtcctgtggggatacagacacggtctcttgttttcaaggaccacccagttcggcaagtggaggtaagtcatggacaaacatggtggcaatgctcagtggcgagctatccagcacgtcagaagtccagcccacgtgctgaggaagtcccaaagagagcaagatcacttctgtctgggtgctgctcttggaaggcctcatggtcttccaagacccccatctgacctctgacccttggcagccaaccttccccaggccaccccagagcttgctggcccttcccacttatgcacgccgcctgggcaggagaggtcactcactctgtgtcagggtgaccgtggctgcggctgcggtggccgagggtccgagggccacagggtggatctctgtggtgctgggcaggctgatgatggtggcctcgcccagcaggttgcagatacgttgttgcatgggggtcagcagcactggggctacagctgggccaccgccaccactgccaccgcctgtcccaggccccccagctccgtcctcctcagtgggccccggcgcctcaccaccctccacggcggcccggacctgggcaaccttgcgacggacctcggtcttgaggtcagaccacttcttcttgacctcaggcagctctctgcggcaggtggccacggcgttgacccttctcaggatgccgtgccaggccgcactcttggcggccaggggtaccccggcgttgaagtggttcaccagcaggtgcttcttcagctccagctcctccacgatgatctccacctcccgctctgagaagttcatcttccttttcttggctgggacggccatggcctctcccctcccctctttttaaagaaattataatcccctcaagcgccccaattccccttctcttcttcctcagcctcgcccctcacccaccccctacaggggataggccaggcttgctcgaggccaagcactaggcctttggtaacccccctcgctacgcaaagtgcgtagggcccagccccgaccggcccggccgctgccagccagcagcgtaatggcttcctgaatctgcctcttccgccgaggtgtgcggagatccgcccacttcccctcttcccgcctcccagcgcttctccgggaaaaccggccttcccggttggtagctgctcaaatatcacgtcgatcactggttctttcgatctgtcgctcagctaaagagcacgcctgcttactgtaattcgcctgatcattggtctaaaattgtgtccgtcgcccagagggccccgcccacctaacctttattcacttccattggaaaatcattctgtcgttcaattgtcagtattacgactattaagccgttaaattcagctattttattggtggtttagggagttagattaccaaaaccctgcttcctgaggat

SEQ ID NO: 143 - DNA, 2001 bp, Homo sapiens
gaatgaaaaggtggctcagtttgagacaggggtactttgggcccctgtcctggcatgctgggagccctccggactccattatcttccaggaggggcttgggcccatcctctgcttcccaaaggccgtactcacgataccgacccccttccctccctgagccacagatgtgaacgtcaggatccgcgtgcttggccgccattcccggagcttccgtgggttgattgcaggggcaaagctccgattctgccaggatgggtggggatgggaatcggggtttgggggtcccgttgatgtttgagcgatggtggtgggtttggtgttgggggatgggggtggggttggtgttgggggatgggaatggtggtgggggtgcgggggggttaccctcagtcccctcttggggtcctgaccttgattcctggctgagtctgggccctgagcacttctaaccagccctgccctggtcacacaaagctcttacaccagagttccctgcggttaaagcggcggggggtggcggggtgaggtggtggcgactgagcctgagaagtcttcccctgcagggtggtccaggcctggcagcagaggcagcgctggattattgggggccctctgtctggggtgaggcccgcctcagtcctccctcggcatcttaccttgcctcctcacggagcctgcgcctgcttccctccaccgacgtcaggccgttgctcagggcgagaatctcgctgttctctgacctccaacgcgcaagtcagtggcgtcacatccggccccgtgacttccctgggttcacagcaggggtggaactggattcttcctggatggggatccagatggaggtggaggtaagcatgaagatggtgttgggggtaggggtagggatagggacggggatcctgaggctgtagatcctggtggggttgggggagttgggggtgggaggggcctttggtcatccctcaaggtcctgacttggatgccaggctgagcctggaccgccatcctctgccgattgtgtctgctcccctcagaccaagtctctgactctgagtccccctgaagtggcactgggggaggggtgcggtcggggtgacattattgcctaagtttatcagggctgacaagagggactgagctggattctgtggtcccactacgtgggggagggtggaccctcagtcctcactcaagagggtcctcttcctcactcctggctgttggatgaagtgattggcaggagatactctgtttctgtaacgtctgtgcatttgcacagctgcaccccttgtagagaatggctgcgggtcccaggccaggagctccctgcagggcgggagctcccacgatcgtattgacctctggaagaagacagacactttcccacgggagctcctctccagccagagctacacttggcaaacctttggtcctaaatgatttattcactgaattgaagaaatacggtttacatatcttccaagtatatatgtaggtagcgatatatatatttatttctacgaactaacacctacccttagtatgcatcgttcactcttactatcctaatctacttctattccagtctttgtgttctgttgaaggaatttttttttaaacgtaattcctctcccactatgttggtttcaggttgttattagcagaactctggagccatgctgccttaacaaacccaattttcctggtgtcctgctattacctagggctcttggcatcgtgttttcaggagctgcctcagattgtgcaatgaagtgaagtggaaattttaaaacttttattctacccattctgttatctgccgtctaggcaggctacaaacctccccaacatccctgccgctaaccccctctaaccgacacggcaagtgtcacacacaagtaccagctcaacaaggtcttcagtctcaccctggactggccctgttttgttgtgtagattttcttgctaatactggttttttactttctgtttccattcta

SEQ ID NO: 144 - DNA, 2001 bp, Homo sapiens
ggtccccggcgctgcgtcctcccgacccgctccccgcggcgcccgggtcgcgccttgcggcagtcaccgagctgagggcagggcgtcccagcgtccctggccccggccccgctccgcagcgcgccgcaccccgaggctcgggccccgcacagctgcagccggtgccctccggccaagcccccgcctggcgtccccggccccagccccggccgcccgccgctgaccttttcctgcgcgcggttgagtcgcttctggacgttcttggcgaagatgcccgtcttgatgtcggccatggctgcgggtccggggagctgcgaagagcagagcgcgcagcggggctggcggcggcgcggaggagcgggaggaggagcgggaggaggaggagagggccgagcaagggaggagcgggagaagcggcggggagccgcggagcgcggaggggtgagggcggggcgcggcggcgggaccagcttaaagcgacagagcgggggcgggatgcgtcgggactggctgtgacatggggagaaggggcgggggaagaaagagaaagaggccaagaaaaagagagggaaaagccagaagccagattgtctcctcgatgatagaagagtttcgagaggagaaagggaaccaagcctagcagaagtccctgggcccaggggcagctcgggggttcttttaatgcagtttgtatttggggagagtgaggggacctgattctttccagcaaaaaaagaaataggggagacaaaaaagggaagaaccctagggatgtggaagccagctggattggaactgctctgaagtggagctgatggggctggtcccttgctgctcccaaaagtttccagctaacagaggcccctccatacaccctgggtgttgcagaagaaggtatggaggtcgccctaccctcaggaggctcacagcccagtgaggggccctggctgggaaaccaacgaagtcagagctgcaggagagaggcgtgtggagagcaaagcggggagagaccatcgctgacacttgtttaaactttaaaacaagagttagcagggcggggaggtccgcgttgagaaaggtcagcttccggaagacaggcgatgtgttttggtcatctccaaaattcctgtgctgggcagaaaccttagaatagactactcttcaagaaatggtgcatggaacagtcaagtcccttatagcttcgcatcaaccatggaagacccagtcccccctaacttgttgcttccttggaaaccttttggcaaccttaacagggactcaccatccctattattcttattaatgcttgggagaagtacgaataatagggataaacctgccctacacctagagctcttgttggctctgaccagtggtcaaactattcatattccattctcactgagagtgtgaggagaagaagtcaggtggtgccctgcaaacagaggtcagaagtggtcactcacagtgaatcctatgagtccaggtgagcatcctactgtacctctgggggaaactgttacattggtagacctcagcgagctgccacctggtgttcactcttgtgtcaccctgtagtcccctcaccttgattctagaatgatgctatggccagctttaatcaataagatgtggtgaaagtgacgccatggtaattccagtcccccccccacccacccaacctagaaaccaggcaagttttgccctctggggattcctgtgacaccatataggaaattcagctaattcagctaccctgctgcaaaaataaaataaaataaaataacaaggagaggccatacagaaagggagaggccctgagattacgcagagagagagagagagagagagagcagccaagatgtcccagctgagcccattctccaatcagttcactggctggctgcagccacacaagcaaccatcagcaagaccagcagaagaaccactcagctaatcccagccagagtgcagaatgtgagcaaacaaaacagtttttat

SEQ ID NO: 145 - DNA, 2001 bp, Homo sapiens
ttccccagaagcacgcactcagggacagccttgggttccctcccaaccttagccacagccccagattcctggagatggccccgcactcactcttcttgcaggaggtgcatttgcactctttgcacttgcaggagctggcgcaggtgcaggagacacctgctagaagagaaaaagccagtgaacggtgagtgagatgcagaaggtacagcagtgggtcaatgagtgtccacttcccccttctctgtgcagagccagccctcagagctcctttcccactcagagcagaggaagagaagccccatgtcctctccattcatgctgggaaaaccaagcaccctcctatttctactccaaaggaaagtcccaggctgcagacatggcccaggaagacttcgactttctgtcctgaacctggaagaggcaatgtcccctcctatccaatgtctcctatccaatccggtagctcagagtcaggttagagaattggggctcagccagcagccctgtgaccaaccaggtgacgtggagcaggacagccttgagcctcaggaccctcaactctgacatagaggaacagggaggaggaaaatgccctcaggagcttggccaacaggaaagcactggaccaataaaggagtcccctttacctctgatagcaaaatacgacctcctcaaacccagggacactctctatggtgtctgggaatttggcatcccaaggcacagaacccgggcgtcccttaccagcggcacaggagcagttggggtccattgcaacccgaggcgagactagagttcccaagcgagaagggaagaggcagtgggtgcacgtggaaggcggagtggagcccaacagccagcggctgtttttatagtcaggagacggggccgggcgcagacgccccgccccgcccttgcacccagcccgctgagtccgcaccgcccgcggtcccggcctgggctgtgcgcaggagatgggccaagtgcaaggtcccttgagcgcagctgggcgcacaccgcaggacggcccctttcgcaccggctcgcgagggaggcgctgtgccccccgtgtgcggcttctctcaccctgccaggccttcccagcttccctgaggttgcctgctacacccgccccttggcgttccgcctaccccagtattctggctttcccctcaccttcaccaacgcccaggtctctagtcaatagctaagcctttcctggagtcgagggcaaccccagtcttgtgctccactttctggatctgccaaagcggggagcatagcaagggagtagtgtgcaaagggcaggccaagggtgcgaacttcgcatctctggcagtatccgccctgtccccgttcccacctcagtcctttggtcacgcttctcctgtgctcagtaacacggaagaaacagtgatctgcctctctcttctattctccactcttgaggagctgaatgggcccatttgtccaggtcctcctttgcataaatctctccagggtctccccacagcctagccgggtaccccactacaggatggcagtttggaaagtgtggcaagtccctagaagagggatcattgaaggtgatatgaagatcctgcattaaattagaaagcaagagcgctggttatttttccttccatattcctattactctatgttacacgggtctgtgtttggccctgtttaatcactttctgtctttgaaccctcacagcaaccctaaagggacatgagtccttttcacgaacccgtattcctaatctggacaatgacgaggagttcaagccactagcccagggtcagggagctgggaagtggcactgcttggtgtgaatcctgtcagcttccctaggtagagaggtcagtgatgaagcttctctatggcctaggtatgtttatctcttcactgcttgaaatctttcaacaaaaccaagcattacagtcagtcctggaagcaaaacagagacatagagggatatgaataagtatactttg

SEQ ID NO: 146 - DNA, 2001 bp, Homo sapiens
ggggtccccggcgggcggggccggagtcacctggcagctcgggactcaggcgctcccggggtcacacggggccgcgcacatcagccccgccgacgggcacgggcggctcacatcgatcccctcgcggccacgctcacgccctgagttgttgggatcgcgggcagaagtcatcccgacagccacccgtgcgacgacacagtggcccctcactcgcgtccccctgacagcctcgcagtcattcacaagccgtcacagtgacccacagccacacgcaggcggccaggcactgcttacacggtcccgccgtcccacgcaagtcaccctgacagtcacttatacacaacgacacgcaaccactcacccgcaaacagcctcaccgccacagacgagtcacccacagacgctccgatgccccacacaccgtgacatgcccccacccgctgtcacacccaggcacaacgggggacagtcacacaaagaagtcgcccgacagtcacactctcgcagccacaacgcctcacccaggcgcacgctgtcacacacagccacccccctggacggtgagcctcagacacagatcgtcctgacggccacacagtcacacgcggtcacacccagggacggccacacctcggtcatccacactcgcgcgtcgaggtttaacttctcagcgccgcagcacactaattgggcagtttactaattggccgtcactgcctggcggggcctcggattccgagtaggctgcggccaatcgaggcctgtgtcctcccgggagcggtgagccggcgtccctccaggctggggctgtagtgccggacaccctccctcctccatgtttccaggatctatccccccaccccagccaggtcccagctgcgctgcgccgggcgcattcccagtgatggaggcttgcgtgcctggaaccccgaacccgaggcgtcaggtcccactgccgggtctggcccagcctggatgggttgggtgcctctaagcagtccgcgtccccttcggtcgccggagagggaactttcaaacttagttaggaagccagactgtccgcgcgtccgccagtcggtgcgtcggtcccgggccggctggagccaagcgcgggttttcgtcactcggagcccggattgaacagcgcgcgtgggtttcccgcagccctggcgcagacgcgtgggctccgtggcgatgcggggtgagtgcgcgtccagtggctggatcggcgccccccagggtctctccccaacctcgcaggcttttcgtcaggcccctggtagtggggatgaggcaggagagaggcgtcccggcgcggatcccggggcctcccgggctgggcctccctgagtgcgccctcaggccccaggggagcggcttctggcaaagcctccctgcagttcgccaggggcagcaagcatccaggccactagggcctgcgcgatgtggggcggtggtgggcgacaagtgaggtctcgcgaagagtggcggtggttctcttttgtccagtgacctgcaaagttagaagccgatgaggggggatggtagcctcaggagaaggccgggagctgcctccagtctgatggaggaaaatagattaaaagtagcaagtggagttttcagggaggagaaggttgggtgtgggggcagctggtctgtcttctgcccttggctttgttatttttcaaaatgtgaagttggcgaggtaggtaccagcctctggtttgcgggtgtgggtgggctgggcatttgtgcctcaaagtctgagtgggtgggggtttagcctggagcgctccgtttgagggtggtgtttctataaatctcgccagggtggggctgtcccaaataggggcctttggaggcaggactcagccgaggcccctgctttattctgatgctctgtaaacctctgactcagcaccagaacctgtgcgaatctcgccgggaagggcccgtcagggagggaatgagaagccggcgattctggtttctttctgtgtg

SEQ ID NO: 147 - DNA, 2001 bp, Homo sapiens
accccatcaaaaattgggtgaaggatatgaacagacactaaaaagaagacatttatgcagccaagaaacatatgaaaaaacgctcatcatcactggtcattagagaaatgcaaatcaaaaccacaatgaggtaccatctcatgccagttagaatggtgatcgttaaaaagtcaggaagcaacagatgctggagaggatgtggagaaataggaacgcttttacactgttggtgggagtgtaaattagttcaaccattgtggaagacagtgtggtgattcctcaaggatctagaaccagaaataccatttgacccagcaatttcattactgggtatatactcaaaaatcattctactataaagacacatgcacacgtatgtttactgcagcactagtcacaatagcaaagacttggaaccaacccaaatgcccatcaatggtagactggataaagaaaatgtggcacatatacaccatggaatactatgcagccataaaaaagatgagttcatgtcctttgcagggacatggatgaagctggaaaccatcattctcagcaaactaacacaagaacagaaaaccaaacaccgcatgttctcattcataggtgggagttgaacaatgagaacacatggacacagggaggggaacatcacacaccaaggactatctggggatccggggccaggggagggatagcatttggagaaatacctaatgtagatgatgggttgatgggtgcagcaaaccaccatggcacgtgtatacctatgtaacaaacctgcatgttctgcacatgtaccccagaacttaagtaattaaaagaaaaaggaaaatcaagtgattttgcttttgaaaattcctgctaatgtttttcatgcaacatctctttcgtgtgtggctcaggggtagaacattgcatcaggatttgctcatgggtctcatgtctcttcctcctcccccagcaaatgacggctgctcggaaacttttataaaggaagcggttgctttgtggctgatagatctgaaggtgaagagcttctggccaaaatgattcagcagcaaaacactcccctccccagcaccacgcaggcagccccctagccccgcccctgccccatcccacagtggtttgcagggtggggttccatgctgtggccctggcacctgcccctgggctgggacagcccactgttccatgctgcccaagaaggctcagcacaggcacaaaccattgcccggcactggcccgtgctgcctgagaaggattggcacgggcacagaccactgcccccacctgccctgcgccatctacccaagaaggctcggcacgggcaccaaccactgcctccaactgccccatgctgcctgagaaggcactgcacggccacccccaactgccccgcactgtccctacccgggcagccatgcgagcggctggaactctgctggccttctgctgcctggtcttgagcaccactgggggcccttccccaggtaagtggcgttctcccctgtccgctccccgggggtctctccctgagattggccactggtgccttgggtcttccccagggcactttcctagcttggagcccccacaggaccatgcggtggtgagaggtgctgggagtggtgagaggggcaaggggctccatctggctgcagctacctttgggggtagcaggctacagggatgaagggtttgggggctgccccagaaggctaggggcaagggaagcatcttctattctctggctcttcacagaggtctgagaaaggtctaagtaaggcaggtctcccgcagctcctctgcgggacggaggcgacagggcagccacatgcctgtggttagccgcttctatctgatggcttgtcattccttcctgctggctgttgtcctgtgacttggctccagggaccctaacgatcagactctttcacccttcctgaaatccactccctgagggtcagcactcagggggaggggggacagggagga

SEQ ID NO: 148 - DNA, 2001 bp, Homo sapiens
agtgatgaaaaaaccacaggctggtgatgcaggtcagaggaggagatgctctttcacattcggacttgagagagcccgagagtacccataggcatgggtcaagagagctacagtggagctcttgggggacagaaaggaggatgtagaaaagaaaggggcagagactgtgcctctagccacccatggatacataaggcattgaatccattagtgaagagaaataagtaatgaagcatctggggccccgtgtggctcctattataatgaatcatgtatgttctcatattagttattaagtacacctggcttggtatatccgatggcctattttgattaatttatctaattcttttttttttttatttcctaggccttgggttaaaagactcagctatcaacctggaggataactgattacctagtcagagttctggtacctggagacaaactaactttgattaaagcatcagatcaccaggtcttaaacactgggcaccaaaccggtcctgggacaggggctaaaccttgtcagttatgatcagggagcagtcctagaagaaaatagcacccaaaggagaacttgctcttttgggaatgagaaagagctaatgttttgcactatggattcagtcaagaagagaaatgcttttggggctttcttaaaaggccaaatgtaaaaagtacccccatcatttcctaaaactcaaacctctaggactctacctcttgggctaattgctcatgagccaccaatctgttctgtcttgcactgtgtgttctgagagccagcaggttaggacagagggaacatgacggggtcagaggaggggctcacctgactgcacagctccatgttttctgagtacacagcaatctgtcgtctcggctgctgctcctctgacagatagctggagaacaggatgaggacatcaaaaccaaacttgtctgtaaatgctttcaagtcactggtaatattgctgtgaaatagacaattctgaaacgacaacaggaaatttgatattaatgtcaaactgcccagctgcggggagttgttccacaaatttgtccagtcttatgatgtatttaggagagatatttattatttaaggcaagtgaaatacagtttgctttgagaaggagtggaaactttcatgctatggcttaaaatacactttgcttgtttgttttcccaaaccatgtaagaaaggagtttttgccattattaataatagtttagacaaagaacagatatcctaaacatttttttctaaaatatttattcagatgcttaatttgcaaatcctattttccctcttgtcgcttataggatctatatacgaatgtctgctattgttacaatttatcacaataagaataggaggaagttatttcttcctgtattttttttctaatgattattattaactccaaggagaaattcctacgaatcaacatagctgagtttactgggtgatctttattccagtgtagaattctgggagatttttaaaagagctgtgatgtcagatttgcttgttctatgtggcagctgtggaggaatgaataattgcagaaagggatcccaatacctcagattacccagatgtggctcatatcatttgcaaaaatgttctggctgcttcaagttttatttactgtatgtatgtacttatttatttatttttgagacagggtctcacactgtcacccaggctggagtgctgtggcgttatctcggctcactgcaacctatgcctcctgggctcaagcaatcctcccagttcagcctcccaagtagctggggctacaggtgtgtatcaccatgcccagctaatttattgtatttttttgtaggcgtcaggttctgccattttgcccaggctggtctcgaactcctgggctcaactgatccacccacttcatccttgcaaagtgctgggcttacaggcgtaagaccgcacccggtccggtctttgagttttattaactacaatgagagcc

SEQ ID NO: 149 - DNA, 2001 bp, Homo sapiens
acctatggcttgcctggtttacactttctgtttcaggtcagccagaaagacgtccatcctacttcctgaggtttttcgtgtaatgagacactcacgtgtacccttttccacgagcgttttattcccactctccaaagcgaggtcttcctcccctctaggtggtgacctacaaagtgacctaaaatcctgtcagttcctatctttgaatactggagtttctttgatccaaaatacatttgtacccagtgctcaaatggctcatcgaaacatcagtttgaataaatcatgaaccaaattgtatccccgacatccagggccatgggtgacagagccttgttgtcttggagcataagggaactcaggccagtgcctgacactggcatcataaaagattccattgtattccagagagaaacgctggcctcccccaatttggaggctccttcttctccagtcccatggatgctctgcagcctgcacgttccaccctgacccctctgcccccagctgatgtttgcctgggccacattttttcagcgctagcatcacccaatctttctcagtgctgtggactggccctagctccctcttctgcaaccctgaagcagaagcggcacctgagaacacctcttgggctgcttacctggatctgcagcttggtgtctgagagagatggaaggccagtttctgcaatcactcagctcccggggtgtggaccttatacagattacaaaggggtttggcttccaagaggaaaagggtgggaggagcatctggagctcagttcaaataaaatcaaggacgtgaggaaggagtggtgaatcacccaggccgccgtctttctagggctgctgaagaaagagcaaatctttgcctacgttagtctttcttcctatgtgaaccacatctgtctgctgatgcacaaggagcagcagagaaagccactacagcactggtgcccaaacctggcacactaggaacaaagtccttgttacttcttcgtgggcgttttcaccaagtagacttggaggttaacaaaggacgcaaaggagaggttctaatgggagatagggaattctggccaaagctattccagggctcctagggagcaaaacccaagtctctttctaggagggcattctcccaacgctacctctcccggggatgggaggcggggtcggggggaatggaggctgtcctgctgcccccagctggaaggcttcccagatctggagaagaaagggggagtaactgaatagggaggaggaggaggcctctagggtgcgctcacccagttctggccacttcccacccaccctccacccctgctctgggggcatcgagaggatggggctgttcttatctacagtaaccactgaccctgtaggtcggaattgagtagaggaactaaagataaacttaaatcccaaatctcaaattctttttggggaaaaaatacaaagaatatcatatttattagcagcagaatgccctcctagaaagagacctgtttttttgctgagtaacaatttggatacctctaagaattatcaaaatatcccttcgtgctgtgtattcagattgtttcagcctttctgtgtaaaaagtggccatagtgaaattatctgacctaccttgtgtgattgtaggtcgtaagatcctcattccagagagggtcctgccccccatacccagaaaaaaaggacgaatgctcagagaggccaagaagaatctagacagggaggccttgctgggtttccccactcagtctattttcattagatcgccctgttgtccagtcatatttctacacagctgttcatactttgttgatatccaagtgtaagaatgcccactttcccctgttatctttgggtcttctgaaggctcccgtgtcgtgtaaaactacgatcaaatcaatttgtatgcgttttttcctatcaatctgtcttttgtcagttgattttcagcaaacctttggagggtgaaaggggaattttt

SEQ ID NO: 150 - DNA, 2001 bp, Homo sapiens
ggactgtggattctttctctggggcaaataaaaatgtagaatcagaaaattcaggcactttgcactcctcatgggacactccagcagcactcacgtgaccatcctgagaatggacaggacacctgaggtggggaagggagcacagaacccagacaccagcctggacacaggcacctgggataatctcctattccttggaaagttccagtatctgagggaggaacagtgacttctggtcctgacctgagtggagaccgagggactcagaagagctggaatcagacccccacacacactgagtatgagtcagagaacaaggcctgagagaaaaagtcacagtgcccaagactgctgcaggggtcaaaggggactgctgatcagtgttccagggatttgctccagattaatctgagtcatggaaaaactgcctttcttttgtttctaaacaaatacctgcaaagacaaaagaccacataaatccccaggtgcacaactttttcagatttaaagaaaaaacaacccccgtctttccctccattcccaggagaagctcactctgtggcatcaagctgcctgggtgagctctcttctagaagagtccagggggacaggcaaggaatgggaggcaggaagtccagttcagggagggggattctgggatgaaaagtgaagggagagggactgagcccatgctgagggtttttctctggtttctcagacagctccggggccaagattcggggagacattgagacagagcgtttgatataaaagaagggggtaagagccaagtcccagggcccggagcttggctctctgggcttcaggccccgagggcggtgcctgggatgggccggctcagctttggggtttcccgagctccgctctctctcttccacctttcccaacctgtgtcaggtccttttacctggatactcatgatgcggcctcagttctcacttccattgggtgttgggttcctagatcagccaatcagtgtcgccgcggttcctggttctaaagtcctcgccggcccaccgggactcagattctccccagacgccaaggttgcgggtcatggagtcccgaaccctcctcctgctgttctcgggagccgtggccctgatccagacctgggcaggtgagtgcggggtcgggagggaaaaagcctctgcgggaaggagcgaggggcccgcccggaggagtaaatctgcacatatatttaattacagattacaattacaatcaaggcagaaatgatctcatttttacattacaactctggaaaaggcaatagactgagatgcaagtgtgcccccaagtgatgggcagaaggagagaaggtgttttggatgcattctagaacacaggtaatctaaggagagttgatcaaggccgtggaagagtcctccagccactattggccatcaaaggagtcctctgtgtcccaggaatggtcctgctttggtgtccctggtgtgacccatcacccgctgggaacagcctgagagaagtagggcctctgcaccaatgctgctgaggatgtcagagcacaggaacgaggccttgggaaattacctggaaatgcgactgaaatcttccttcctgaggggtctgggctcttggaaatcaaaccctctcaggttgggtggctggacgattctcctcacacttacaatgggacaaggggaaccaggaggcccccaaggggatccctgggttccacacgaactcctcctaccctcattgtgtgacagcagccatgcctcctcctggggatcaggatctattacctgtgcctggagaggaggggactcctcttctcacccgctggtctctggacacatactgtccaattcccctgtggcagctgtaatgtgtagttcaatgggcactcatttgtccccttttaagggtaccctcctttagaatccaggaccttctaccctgcagagtgtggttttgggagagaagtgcaaaatcccacgacaggtgagttga

SEQ ID NO: 151 - DNA, 2001 bp, Homo sapiens
accattcatctgcctccagactttgctagtcctcagagacactgctgcgagggtagtgggcagtgggattagcctcccgcagagctctggcctcctccagagcttctctgtatcgggagggccaggcctgggggtctttcttaaagaccctggccaggaactccatgatttgcatcttggtgatttcgcggctggcccgggagccccaaaagaactcgtattcgggcggctccacgtatgggacgcgctggtacttcaggtaattcatttggacgaactcctcagtgatgagcttccgcacgtccccgaaggtggagtgcttcttccagggccgcagccccaggatgcgcagcacgttccagacggcgctctctctggcgccgcggcccttcacgtagatgaggctcaggatcatgagcaggaggcctgtcatgggcatgcggttgctcagcgccaccctgtccagctcctcgggctccagcgctttgaccagcgcaaactccatggtgtgcaggctggttagcctcaggtgcagcccgaacacccgggcgaggatgaggctggtgcgccggaggatgctcctgcaccacttcttgtagctgccgatgacatctttcaccatgtctggaaaccagatgatcatcttcttctggtccttgaccagcacgtaccacatgagctcgtgcgccttctgcaccagctgcgccggggctggcggtgccgggcccggctgggccgcgctcggggcctggtgggcgcggccctcctccgcagcctgctgcagggccttcgggtcgccctcgtcgttcggggcctggggaggcggcgcggcctgcggagcggccgtcgggcctagaggagggctctgcggctctgccagggtcgcggacggaggaaccccctccgaaaccccagggctgctgtgcacctcggagttgggggcctcggctgcaaagttagggtcgctcagatccttactttgttctgacatgtctgcgccgtctggcaagggcagggcctctgcgtccaggagctcttcgagcctgcgctccctccgcggattcctggagaggaagtgcgcgttgctgcgcgcggcgccttccgcagcgacgggcagggcggggcggcggcgggacgcggcactgcgcctgcgcgaccgcggccttggccagcggctgggggcgggcggggcagcgcggggccccgacggggagggtccatggcgaggcttcacctgctgggagagcgatcccggggtttcctgtttgggctgagaagatctagggcaggagagaagtgagatggatgcgtactggagaaggggccagtttaagggaaagataccttaagtttttgggatgttggggtcaagtttcttgggaaacgcttacctgggtatatccagaattaagttaaatatgagagtcccagagaaaggtctgggcggctttgtaaacatgagctttatgcgaaccagtgagaggaacagtcgggcttgtccaagaaaatgtgtaaactgtaaagtggatgtctgaggttggaatcctgagaaattccaataaaaaatgatcgactccctaccaaatcagataggctataagaaaaggggaaatgcagtgtgtcgttaacgtaatttcctatcaacccatgtgaactatgaactttttaagtaggcagtggacttagagcatcattaatgctttaacgggcataaatttgggttttacacttcctttgggtaactttgattccattttatttttttccatacttagtccatagcagaaggattgctatatcttttagcacaaacttgtatgataatgtcttttccctctgcccagcaccctcattcttcaattcaccttctctcaggaccctctctgaagctactgagtcctccacgttggaagccacacccaagtctttcctgagatttaaaaaaaaaataggtcccatgggtaatatctggtagtgtctctgagatgaccaaagggtagc

SEQ ID NO: 152 - DNA, 2001 bp, Homo sapiens
ctgggatctgacaactgagaagggtctcagcccagcctgcccctcccctgccgaccccacaatcaccctcccatcctttagcctcctccttccctctctgtctctcaatgtccccttgcctcttctgctcctcacccacatcccccacacatcctccccaacccggtaggctgaggggaggggagagaaagtggagggggtgctagaaggcacatactacaagcaagacagccactcagcttatctcaagcgaccccacagcatccccagggcataggcctcctcccagatgaataagctgaggttcagagaggtgctgtgacttgcccaaggccacacagcggcaccaagccagtgacgacagcccaggtcagcctggggacaaaggctgaatttcaggtttcccacttccctcctgcatccacagagtaaagactgaggcttgcctccgtgggagacctgcccccagcttttggggtgcaggtgaggagccctgggcactctcacccatagtcctgcaccctccaccgagatggcttctggggtggggtggggctgaagagggtgaagctcccctcagcccacccccgggtgctccctgtgggccctcggagaaggaagcgtcccctccaagctgccttccagggacgcctagttctgcagggccaggggcttccccaggggcaccagggcggaaggatctcagggcatcccagttgggggctgtgaggaagtccagccaggtacctacccatctcccaccaccacacacttgatggcctgcatcgtgtccggagcctggagagtgtcggtggtgacagctcagggccaggcgcgtttctgcgggcgcaaggggtgtggaggctggtgaggcgcctgctgaggagcagcggtggtggggcaggaggaaacggaaggggaacttactcaagctgctctccccacggtcgtttgcagggggcggagcatgcggtctggcagagcagctggtgggaggggcgctcaggtgctgggctgagccctggccatccccacaggccccctgcgtggaggctccagcaggagacctgggggccctctgtgctgggaggctcacttcctgtccacggggcgggcccagaggctgttctccaagaacttgagggttccaagaattcagtgcacttcaattctaagcagagcatttaatgagtgcctgctgtgtgccaggcacagtaccaggtgctggtgatcccaggatgcaagagacagctggtccctggagtttataccctttgcagagagggaggcatgacttcatcatgggagatcataacagagacaggaagtgattgaggcaacgggggaatctacagagaggacctcagggttaccgagatggagagctgggggcctgcaagctagcattggagctggctcttgaaacgtgcgccctaaagagggaactccaggtggggggagattgcttgagccaggacccagaggctcccccaggcaagggcctgtgtgggagtccctgggtgggaggatgtggggcgatgaggcaggaagggaaggtggggtccgctgatggagaagggcacattgaggggaagtggcgggcagtgccccacaggagccaagcctattctgaggccgccctgggggaccaggcaccctggagctagaactgtggcccatttccctgatgcgaaaactgagggtctgagaagtcatttgaccacctagcctgaaaccagccctaggctcaggtgactccaagctcttctccaagctgcagctattggctccggctgctgtttgaagggacaaaacctgagttctgcagcttgcagggcgctcccttcgccacgagggctctccacgtggactcgggtgaatattcgaccaccccagttggcaccaagttcactgttcattgctatcactccccacccacacacctttccagctccggggggaagggacggaggggcggggagaggaagagccactggtcac

SEQ ID NO: 153 - DNA, 2001 bp, Homo sapiens
tcaggaggccagtcacttgcctgtagcccacagttagtggtagcactgggattccaacttgggtccccttcatgtcgcctgtatttggtgtcccatttaggcgtccatgcaggttttggcgtgtctgaagatctgtgtctccccaagtaagtgtcccccatggatgcccacccagccgtgtcctcctgtctctgaggtttctccaggaatcgagctcctggcctggcttgtctgagtgtgtctgtgggccagtagcatgcccctgcccgtctgggtccctctgcgtgtctctgcttgtcctagcctgtgtgtgtcagtgacgtctaagagtgtgtgtgcgtgcgtgcgtgcgtgcgtgtgtgtgtgtgtgtgtgtgtccttgtccgaggagccgagagagtgagggggaatgaagggccaaggaggcctggctggggctgggctgggggtggaggcggggagaggggcgtcacggcctgagctgagaggggagtggagttctggaggaatgtttaccagacacagagcccagagggacagcgcccagagcccggatagagagacacggcctcactggctcaggacagggggcacagccaccagggtccccttccccctcctcagctccctccctggcccctttaagaaagagctgatcctctcctctcttgagttaacccctgattgtccaggtggcccctgggctctggccctggtgggcggaggcaaagggggagccaggggcggagaaagggttgcccaagtctgggagtgagggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccaacaacttctgaggaaagtttggcacccatggcgtggcggtgccccaggatgggcagggtcccgctggcctggtgcttggcgctgtgcggctgggcgtgcatggcccccaggggtgagtgatgggggctccttggggcagggatcccctcggaggggctggggcaggggtaggaggtgggggatgatggcaggtgtggggggcctgggctggcttagggagtttccttgggaccacagaaaagggacttcaagggagggagacacagactcagagacaccaagtgcggcagagggagagaaagactgagagaggaactgaagggcaggggggacagaagagaggacccggagagacagacagacaaggagaagctgagggcaggggtgggggctggacctgagaagggggccatggggagaggggctgagctgagggtcgccaggaaggaagaaatctttccgaaggggcaccgagtcaggcagtgggatgaggagggctctggagccctgggggaaaacgcttctccctgccaacaccacccccctgacccgccacttgtccctaccccaccagtccactccatctctggggaggaggctctcactttgtctctctgtctctgcctccagctctgctatctctgtgggtctctgtttctctctctttcacagtctctgcctttatctctccctgctctctgagtctctctctctcttctcagcctccctctgtgactgtatcccccctccgcccccaccttttcttctctcttgagggctgtttcttcattttttctgtcttgccttgtatctgtgctcctctctgtctcatctcccctgcctttctctggcttctccctccgccaccacccttatctccccccaccaccacccttatctctctcccccaacccctgattcttcccctccatccaccagcaccagcgcgacctgttaagtctctagagacctaagagaggacggagggggcaggggctggccctgccaggcagtgcagggcgtgggaggctgccagatcccgagcctccttcccgacgggatg

SEQ ID NO: 154 - DNA, 2001 bp, Homo sapiens
aaggtaaggccttttgaaaaagccacgatgcccaggtgagggttgaattgatcactgagaaccctacaaatgcgctcatggcaaggctgggaataccctacacttaccctctcggtcatttacttcagagtctgcggggcattctaatccgcgttgtcatttgctttgtgccacattcctaagaggtaggcagggcaggttttatcatcctctgtttacagatgaagataatgaaggtcacatagctcaagtgagctacccagggccacagacatacaaagtggtagaaccagaatttaaatcttggttggtctgactccaagtgtagtatttgtttcactttttctttctttctttctttcttttttttttttaactgtttctgcattctgggttctgaccaggtgaccttgaatcataaagattcagggcctgtcttgagctgctcaatatggaaggcatcttttctctaaagagtgtgagctccctgccaagcagcacaggcgcttaataggctttcagagcagcaattctagaacctcccttgtggctttctgatctcccacagcccttgtgtttgatggtcaggaaaggagaatacccttggatcgtcttatctccagaccaccccgaggctggctggacttaaggggtgaggaaagacaagatgggtgagcagctgctttcggctgaaaaccagaaagaaatcccagtgtttttgctttattttggtttaaaaatcaagtcgtatgacaatggaaacattttcctggttgtcctctcctctctgcccatgttgacagaagacggacttgctaattctggctggggtttttccctgcctgggctgtttttctcttttatcttcatcaattgaagagggctgctgtgtctttcaaactgtcggaaggtatctgtatttttttttctttcacataattctgtaggtcttttcccagatatgtatttttctctctaacttctaactcgctgaagtttccgcccatgtgacttccagcgtgagttaccagaaaccacaagagagagagagagcgtgcaagccccaaagcgagcgacatgtccctttggggagcagtccctctgcaccccagagtgaggaggacgcaggggtcagaggtggctacagggcaggcagaggaggcacctgtagggggtggtgggctggtggcccaggagaagtcaggaagggagcccagctggtgacaagagagcccagaggtgcctggggctgagtgtgagagcccggaagatttcagccatgcctcacagctccgacagcagtgactccagcttcagccgctctcctccccctggcaaacaggtagagtcctcctttttctctctcctaccttctgattctcctgggggatggaaagagagccaggcttccttgtcctgcccagggagctgaggatggaggaagtggctcgttgcacgggcactctgttagacttaggacatggaatttgctactaagctgtgcatattggcagagatcctcatccttccacccattctgccaaagcccctttttctctccattttccaaggctgcctatcacctctgcctcactggggttgccaccctaaaaagctttctaggaagcaaagaggaggatgaacatcaaagaatgcagagaaaagagtctactgttctccaaggctgtagaaaagtgagaggagttttcaagtgggccgctggcatgcacagctcaaatcctagatatgcagtttcccaggagacagccaatagggaaggggagaaaaggggttagggaaaagttaaaaaaaaaaaaaaagaagaagaagaagaggaagaaggaggaggaggaggagaaacccaggctagcatagaaggatctctctactggggcactgcctctgttgctagcacagccagcaaggggagatgctagaggtccagccaatatctctttgcttgtggcttctggataggagcaaggaaatgaatgaggaggt

SEQ ID NO: 155 - DNA, 2001 bp, Homo sapiens
gctccccattgccaggtgcttttcttcctcctcctcttcttcttttttttttttgagacagagtctcgctctgccgcccaggctggagtgcagtggtgtgatcttggctcactgcaacctcctcctcccgggttcaagcgattatcctgcctcagcctcctgagtagctgggattacaggcgtgtgccaacatgcctggctaatttttgtatttttagtagagatggggtttcaccatgttggtcaggctggtctcgaactcctgaccttgtgatctgcccacctcagcctcccaaagagctgggattacaggcgtgagccaccacacaaggccctttcttcctcttctgtcatctgtggatttcccatctgcctcagtggagtccttgaccttttcctccctggcccagcagcacattctcctctcctcctttctcccaagtcactgacctagtatttagtgtgtgttctcagcctcaccattgacttgctgtgttaccttggacaagtcactctcccactctgacctcaattttcacatctctaaaaggagactgctagactagacggtgagtgaagtcctgctgagccctccgagttcattctcccttgtccagtcagtccttccctcccaccctcccctctgggctccctctctggggccctctcccccaggggaggcctgctccagccccaggggagctgcggccctggctcctgttcccagtcggagtgaccagccctgggcctccttcccagcttcggccctggctccatgtggcccatctgagaggctggcctcaagcccggcggcaactccacatttctgtttttccttttttttccctctttcccggagttaacaagaagcagatgtggcgcacgatggttggagaggtggggggaggaagggggaggccggaccgccagccagacaggggggaagggaggggagccagcagggaggaggaggccagggcccgccccacagccactctcgcgcctccgaacagccacaggggcaaagccctgtcacccccaggatccggtcatcagggaaagaggacagggagaccagaagagggccagctgggacgagggggcggacgcccaggaggcaacttctgagacgcagctcctgagaggggcagggaccaggcgcgggaggccagagggggcacagagaacaaaccccctcagaagtgaagaggagagcggaaggaaccgagaggggacggacaggagctgaggaggaaagaggaggggagaggggtcaggccaggcagccaaggagaagacgtgtggccgggggctatcagaaggaaactgggacggacgggccgggctcgggctgtcctgtggagcagcagcatccccggggccggcagaggcgccagtggctgggcgggatgagtctctgagggccactgtggagcgccccgccatggccccccgcaccctctggagctgctacctctgctgcctgctgacggcagctgcaggggccgccagctaccctcctcgaggtttcagcctctacacaggttccagtggggccctcagccccggggggccccaggcccagattgccccccggccagccagccgccacaggtaagagtctggatcccagcccgaggcttgggtggtgaggaaggggtcagaatgccacctctgcctggctctctgctgtgtccgctaatgcagggcagagggggagtgagaagcccatccatcaggtggctcctcacatgcccaccttcccacggggccttttccaggaccccctgaacacatatgttccttgggtcacctagggggtccgctcccacatgccccagggaagctgccctgaggggagcgggccaggccttcctgccagctgccctctgtatcctagccccagagcctcagggagaacttttgtccccttctagggctcccttgaccccatccagtaatccataattctgacaccactccagctctgttccctccagc

SEQ ID NO: 156 - DNA, 2001 bp, Homo sapiens
cccaacataaaagaacgcaactagtaaccataaagtgtttaaaagtctgggcttctccccaccaagaaaagacgagttgtaatacctggactgtgggtctccagtccgacaagaattggctctctgcttcactggtctcgtaaggtcactccctagaattggctttctataggaccatttaattgagtctatttgaatcttactcaagtctaccaccttataagtatcaaaccgaatttagtaggattcgaaatggtaaggactgcattaaattatttctctaattcagcagtaaggaaagtgaacagaagtctcggtgtacaaccacgccccgggttgacatctttccttccacgaagacactgggtacaagccttgatgtagctgttaattgccctcctgtttcctagaaagcttatagggcagcagtaggaaacgggttaggtatgcctttcctggtaggagccgagctttgggtcttatctctgccacttgttcggcatctgatcagtaaaaggggaccatgatgggaggcgaggcagtagacagcactttgtgacggttcctaaccagtcgctgttatcactgagcacattgtcctcgtaatccagaattcatactgccatttgaatattttttgtatttgcaaagaaaattttaaccacttaactcaaggctagaaataaaagcaattagtgattgcaagagaaatccaccggcagaaggcagggggagacgacttaagcctctgattatttttggaagctttatatgctttctagcctttgtcattgaatggattaatgtatgtgcctggcatagactaagtgctttgcttgttaaactgcccgcgggtcatagcgagcaaactgggggagcgcccagggacagtggatggagtgagtggagactcgtgtccttttaagaagaggctccatttcttcctcgcatgtcaatgtgtgcgtgtcatcagttgttcgaggaagctcgcgcatgacgcgacacttgccgtctgggctctccaggttcggcctgctgctttcacagcccagatcttcggcgtccccgagacgcagccaggcgcccggcccggcccggcccggccctctgcacagccccacttcaggccacgccgtttccttgaccttcccccggcagggtctgaagcgtcaccgtccgtctagagcagagtcgcgcaggcccctgccctgccggtgaggtcggcgccgcgctccggagagtcggttcctcccgattcagacccatatggcttcagatggaaccatcatttctgttccgtcatgaccggcttgcaaggtagcaaagcgaaacaaacgtctcgttttcagcctgtccccgccaccttgaaataaaaccggcaaaaataaaaagagcattctgggaagtacttcatcatttcaacgctcacgaattcaaaacataaacaaaggcttccgagtgccccggccaggggcgcggggcgcacggcgggcccggggcaggtaagcgcaggtgcgcgcccgcccccacccccggctccctccccatccgctccccgctccccttcccctttcccttccccgcccgctcccagccgccgccgccgccccgcgcacggcctggagcggaggctgcgcagggcgcggggcggcgcgggccgggcgcgggccgggcggacggccgcgtctttcttctcctggcggtgatgtcattgggcgacggcggccgaggccggggggcggcggcgggcgcccgcaggttcccgagccgctcctgagaaggcgcctgacagcgggccggggcgcacggagaagcgggccgggccggacctgctgggccgcgccgagccaatcgccggcgccggccgctcgatgggcgaggcggcggcggcggcggcgggggccgcgggccgggccgccgctccgaggtgaaggcgcgcgcccctccccgcctgcctcccgggccgcagcgatgaattccgccgagcaaaccgttacgtgg

SEQ ID NO: 157 - DNA, 2001 bp, Homo sapiens
cgggagttcgtgaccagcctgaccaacatggagaaaccccgtctctactaaaaatacaaaattagctgggcatggtggcacatgcctataatcccagctactcaggaggctgaggcaggagaatcgcttgaacccaggaagctgtggtggcggtgagccaagatcgtgccattgcactccagcctgggtgacaggagtgtgaaactctgtctcaaaaaagaaaaaaagacttgatcagcttatagcctttatcatctagtcttctattctctggagagtcagtggggactcttccgagttcttgtagccttttcttaaggcctataggaagtcatctgtgctgagcctgcactttctataacagatggaaaactgaagctcagacatcttaagtggcattgacttagactatagagctagttagtggggggccagcaccatgacctggcccctacctccagcaggcttcacccacccttgcacccttgcaggaagcacttagaggcttgaggttgccagctgtggggggttgggcctgtgcagtgcctctcagccgcccacgctggtggggctctgcagtgttagcttcttgtttctctattttctacctaggtttcagtttttcacttgggtttgtttcccctttgtctctggttcagcttccttttcctgtttggttttaagtaggctataaaaatcaagttgctgtcttcagagggtctgtggtcctctgatcaacataggctggtgggagtacaggactcgcctcctcagggttccctgtgctgccacttttcagccatggccacaaggtgagtattggaaccagtgcaggggacagagggatgtcaagaggaaacctagaatcttaacggtgaatgggctgtagaaattatctcaggtccccttagtatactgatcgagaagtccatgaccagagggcagggggcttacatgaggttacacagcaggttagtgacagtctgctccctctctagccgctggtctttccattacacaggcctgtgtctcttgatgcctgtgacctttaatggcttctgagtcaaggacttacttagaaattagcacctgcctctcattatagggaggagggtctgagccatctaggggaattgttgtttagctcagaggtacatttggtgagtcctggcccttctctctagaatgacccgagactggttgttttttctaaatataggcagagagggttctttttatgataggaacttcatgaacttaatagcaagagtcctgaccctccacttgagagacttgtggacttaggctattgctgttgagcacatgtgtgaataccagccagtctacaaaccgcactcctcattgagattgttctctctaaaggtgagattacattttggacctgcctctcccctgccctacaactctgaaagattatagatgataggacagacagacttttatcctgtggggtttggggcatttgtttgattgactcatgacagctttgttttttagcataaagtaacaatccctcactttgaaaattatttcacaatttccaaagtgccatttcgccagttttcttatttgaatcttttagccgtgttagattgatgagaacgctgtcatcactcctgtttcgtagctgagaaaacctctgaaaagttgtggggtttgtccaaggtcacatggcaggttggggaaggaatggaaccagggtttaacgaggttggcctcatccggtggttttcgcagcacgctacacaggcacagaagcagcttgcagtgagttgttaaaattgacgtggtgataggtagaacttgtccagggctgcttattttccagttggctttatttctgatcctgtcattttagtaactttcctttaactttagaggactcgtgagtggagctttagaaaacagaatgcttttcatttcaaaggcatatcaacttccaaagtcattttcagttgtaaattacattgtgaaaacttc

SEQ ID NO: 158 - DNA, 2001 bp, Homo sapiens
gctggtctcaagctcctggcctcaagcaatcctcccatcttggcctctcaaaatgctaggactacaggcatgagccactgcacctggcctggacctgtcattttacagatgaggaaactgaggcccccaggggacaagacagcctgaagtcactgggatctgacaactgagaagggtctcagcccagcctgcccctcccctgccgaccccacaatcaccctcccatcctttagcctcctccttccctctctgtctctcaatgtccccttgcctcttctgctcctcacccacatcccccacacatcctccccaacccggtaggctgaggggaggggagagaaagtggagggggtgctagaaggcacatactacaagcaagacagccactcagcttatctcaagcgaccccacagcatccccagggcataggcctcctcccagatgaataagctgaggttcagagaggtgctgtgacttgcccaaggccacacagcggcaccaagccagtgacgacagcccaggtcagcctggggacaaaggctgaatttcaggtttcccacttccctcctgcatccacagagtaaagactgaggcttgcctccgtgggagacctgcccccagcttttggggtgcaggtgaggagccctgggcactctcacccatagtcctgcaccctccaccgagatggcttctggggtggggtggggctgaagagggtgaagctcccctcagcccacccccgggtgctccctgtgggccctcggagaaggaagcgtcccctccaagctgccttccagggacgcctagttctgcagggccaggggcttccccaggggcaccagggcggaaggatctcagggcatcccagttgggggctgtgaggaagtccagccaggtacctacccatctcccaccaccacacacttgatggcctgcatcgtgtccggagcctggagagtgtcggtggtgacagctcagggccaggcgcgtttctgcgggcgcaaggggtgtggaggctggtgaggcgcctgctgaggagcagcggtggtggggcaggaggaaacggaaggggaacttactcaagctgctctccccacggtcgtttgcagggggcggagcatgcggtctggcagagcagctggtgggaggggcgctcaggtgctgggctgagccctggccatccccacaggccccctgcgtggaggctccagcaggagacctgggggccctctgtgctgggaggctcacttcctgtccacggggcgggcccagaggctgttctccaagaacttgagggttccaagaattcagtgcacttcaattctaagcagagcatttaatgagtgcctgctgtgtgccaggcacagtaccaggtgctggtgatcccaggatgcaagagacagctggtccctggagtttataccctttgcagagagggaggcatgacttcatcatgggagatcataacagagacaggaagtgattgaggcaacgggggaatctacagagaggacctcagggttaccgagatggagagctgggggcctgcaagctagcattggagctggctcttgaaacgtgcgccctaaagagggaactccaggtggggggagattgcttgagccaggacccagaggctcccccaggcaagggcctgtgtgggagtccctgggtgggaggatgtggggcgatgaggcaggaagggaaggtggggtccgctgatggagaagggcacattgaggggaagtggcgggcagtgccccacaggagccaagcctattctgaggccgccctgggggaccaggcaccctggagctagaactgtggcccatttccctgatgcgaaaactgagggtctgagaagtcatttgaccacctagcctgaaaccagccctaggctcaggtgactccaagctcttctccaagctgcagctattggctccggctgctgtttgaagggacaaaacctgagttctgcagcttgcagggcgctcccttcgc

SEQ ID NO: 159 - DNA, 2001 bp, Homo sapiens
ggggtctactgctaaaagtgatgtgaaagccgccatgttgtaggcttccaaggcctaaatcccgtccctgggaagccagtgaccaaagatgaaatttagctttgaaaccaaatgcagaggaaagtgatgaaaaaaccacaggctggtgatgcaggtcagaggaggagatgctctttcacattcggacttgagagagcccgagagtacccataggcatgggtcaagagagctacagtggagctcttgggggacagaaaggaggatgtagaaaagaaaggggcagagactgtgcctctagccacccatggatacataaggcattgaatccattagtgaagagaaataagtaatgaagcatctggggccccgtgtggctcctattataatgaatcatgtatgttctcatattagttattaagtacacctggcttggtatatccgatggcctattttgattaatttatctaattcttttttttttttatttcctaggccttgggttaaaagactcagctatcaacctggaggataactgattacctagtcagagttctggtacctggagacaaactaactttgattaaagcatcagatcaccaggtcttaaacactgggcaccaaaccggtcctgggacaggggctaaaccttgtcagttatgatcagggagcagtcctagaagaaaatagcacccaaaggagaacttgctcttttgggaatgagaaagagctaatgttttgcactatggattcagtcaagaagagaaatgcttttggggctttcttaaaaggccaaatgtaaaaagtacccccatcatttcctaaaactcaaacctctaggactctacctcttgggctaattgctcatgagccaccaatctgttctgtcttgcactgtgtgttctgagagccagcaggttaggacagagggaacatgacggggtcagaggaggggctcacctgactgcacagctccatgttttctgagtacacagcaatctgtcgtctcggctgctgctcctctgacagatagctggagaacaggatgaggacatcaaaaccaaacttgtctgtaaatgctttcaagtcactggtaatattgctgtgaaatagacaattctgaaacgacaacaggaaatttgatattaatgtcaaactgcccagctgcggggagttgttccacaaatttgtccagtcttatgatgtatttaggagagatatttattatttaaggcaagtgaaatacagtttgctttgagaaggagtggaaactttcatgctatggcttaaaatacactttgcttgtttgttttcccaaaccatgtaagaaaggagtttttgccattattaataatagtttagacaaagaacagatatcctaaacatttttttctaaaatatttattcagatgcttaatttgcaaatcctattttccctcttgtcgcttataggatctatatacgaatgtctgctattgttacaatttatcacaataagaataggaggaagttatttcttcctgtattttttttctaatgattattattaactccaaggagaaattcctacgaatcaacatagctgagtttactgggtgatctttattccagtgtagaattctgggagatttttaaaagagctgtgatgtcagatttgcttgttctatgtggcagctgtggaggaatgaataattgcagaaagggatcccaatacctcagattacccagatgtggctcatatcatttgcaaaaatgttctggctgcttcaagttttatttactgtatgtatgtacttatttatttatttttgagacagggtctcacactgtcacccaggctggagtgctgtggcgttatctcggctcactgcaacctatgcctcctgggctcaagcaatcctcccagttcagcctcccaagtagctggggctacaggtgtgtatcaccatgcccagctaatttattgtatttttttgtaggcgtcaggttctgccattttgccca

SEQ ID NO: 160 - DNA, 2001 bp, Homo sapiens
atcggcctcggagacaggcagctctgagtgagtcaggaccctgcggctctctcacaactcctgaaacacagagattccttgtctttcctttttgctttctcccctaaaaattagtaactacattttaataagtggatgaaatgaattgattgaaaatatgttagtaaaaacatggtacacataattgatttttaaaatcaaataatacaaaagagcctttcctatccagaggcatctattgatctattgtgaacacttttttaggtatcctttcagaatttttctctctggatctaagcacgtttttttggtttgtttttgtttgtttgtttgttttgttttttttttgttttttttttgttttttttttttggagacagggtcttgctctgttacccaggctggagtgcagtggcacaatctcaactcactgagacccccgccctcgcggggctcaagtgatcctcccacctcagcctcctaagtagctgggccacaggcatgtgtccagctaatattttgtatttttttagagaccaggtgtcgccatgttggccaggctggtctcagactcctggactcaagtgatctgccttccttggcctcccaaagtgtcaggattacaggcatgagccactgcactcggccacatttttaacacaaacaggaactgactctccacctttgcccagcgtgctcttttaaaaactaaagactacatctcaggaattgttttataacaacatatcagatgagacagcgatgattctatccattttccagaggaagaaactaagactcaacatttccggtgcctatcagaggttactccagtgtccctggtatagctggtatttaacccactcccaggaccctaagcctccgccctcggcatccgcagcatcccccaaccccccaacctcctgcagtggactggctggtagtgggcaagagactaatctcattccaggaaaacgggcccctagattttgtgttctccgtgtttcttttccagggaggcattttgatgacccccactggcccttccaggagcctctgtctgaagggtggacttggtttatcaccccgagaaatgggagcgcctggctgcttggatttagcctgcagctggggtccccggtggtgtgtgcagggcggccttgagcctccaagggtgcatccacaaactgcccttccccgagtgacccatgggagctcgatggcgtacgtgctgagcaccaggatgtgtcctgccctgtgactcccaggccccgcccttgcgctcagcacgccctttgtggatggatttgagggcccctggcctctgatgtgccactcccgctgaggccagcgaaggaggaagaaggaaagagcgttgcctgccttgttttcggggcactcactttttctgggagacagcggacccaggttcatcaatggacagcaggattttcatgttattaaaaaggtcattgtaggggaaatccctgggggatctggtgagatctccctgggggatctggtgagatctccctgccttcccactgctcctagtacccagaggccaggccaggtcgtggtgcagggctgagttccaggagggtgcaaacaccccaggaacagactgctgcccctccggccctcatgacctgctggggaaggacccccgcaaggagcttctgcaggtgctgtggtctccacctgctgcactcaaaattgcctggagtggggagaatatcaatgcctgggccccgccctgacaggtggttctgtcgcctgtgattcttctctgcggccagcaccgagaaccctgctggggtttgcttccgtttaactagtgaatgttgactgagctcctactgcctacacccagggtttagggctggggaaggaataacaaaggtgacacattcttgtccttgaagagtccccccgcaaagggagacgggatcatgtcagagttaagatggtgaagccaagactaggttctgtgctgcctga

SEQ ID NO: 161 - DNA, 2001 bp, Homo sapiens
gagacagacaaatagcaaggtggtagtggggttcagaatacacaaaggagagatttccgtctaagttttgcctcctgatcatttcttcgaggcgttcttctctttcgtgcctaaattctggtactttcccagatcttgtcagaagaggacaagggaaaataggacaatgtttcctgttttctgagacattgtatatctcctcattctccttatgcatttcttcttcctttttctctcccttccctccttaggcaggaaactgccctaatcttaaccagatgctgcgggcaaggttttttatttttcccctaacaacaaaaacccactgtggctgccgcaaaggacaagaagcctcagatccaaggtttgttagaagaatgttcagactgaaaagcaatgttctatgatgatgaaattcacagcttttcataagtttactctagttagcttcactcatttcttgcattggctttcttaattacaaatcctccattttaagaacaatttgcagcctgcagcatgacatatggcttacacaattgcttatagtccatttacatgtaattttgcaaactggcaaaatgtccagtaggacatttaaaaactagagtgttaagggctttgagatgtctcagtgaacaggaagcactgagctactacctccccataccactctcctcctcctcactaaggagttaaccgttttgatccttgttggccaaagtagaaaacactgcagaaaggccactcttgaaggatggggaaatgattccctatatactttgcttttactaacaatcatgtatgtatatatataaatcatacaaggaattctacagatttccaggcagattttatttagccagggaatgacctaagaagtgcatctctgttttcatctgcagaggctaaaagtttattgtgatcctcaagtcactacatccctaatactcagagaattgacaggaatgaaaagctaaaagatctaggaagttgcaaacgttacctgctgatggaaatctgttgtctggcttcgtttcggcagcaaagtgtgggcacaccatcaggggtgctagatttattccatcgggattgcggaagtcagctccagtgatcggctggaaaatttgggtaagaggaggaggcggggatagaaatgctgagagaaggaagggtagggaggagggaaggaggcagtggatttgcagtgtttctgtgtgtatgcagcatgtgtgccagggtagggaattactagctatttagtctcacaagactgtctttggtggcaaataacaagacctcaatatctattgactcaaataggagcttactttctcttttggtgagtaaaaggaaggacacagccctagtctctcccatccttcttcttcaatatctttattttaatattgggggattatatcttttcctacccctttcctgttaggatttacctctccaacattgaaacctattttcatatttgtgtctgactctcacctctgggatgcggtaatgaacataaaaggctataaaagaataatgtaatggaagccataggctccagaattctttgtatttttataagcttgttggttgcaagttgtcattacttccaggcagaatagttttcccagcctaatgtggttttgctgaaggaggatatgcccagaaagtccagatgaaactgtctttaaaagtaaccaaaaacagcagtcctggttatttatgacagcacttgaatcaatgccgtaagttctgatggactcacatgtgactctgttgctaaaactctcaggtggtgggatgcccgataaacatcatctgtctcaattaaaatccccaatctaggacaagtcacatgagcttctccaaatgcttccccaacatccaagcatgtgacagatcataagaatctagctgattcctcaaaccacaccccaaacttcattttcttccataaacagatgtggggaaggagaaaaaaaggcttcttggaat

SEQ ID NO: 162 - DNA, 2001 bp, Homo sapiens
caagggccttcttgctgtgtcatcccatggtggaacagcaaagagagggcaagagtgtgccaaaaggggggcaaactagtccttttatgaaaaatactttgataacaaacccactcccatgataacaacattaaccctttcctgacggtagagtccttgtggcctaatcacctcttaaaggtgccacatcttaatactagtatgatggcaattacatttcaacatgggtttcagaggggaccaacattcaaaccatagcagtctcaaacactgggctggcagtggaagaggaggctgatctttaaagaatataaattttttttttttgagacggagttttgctcttgttgcctaggccagagtgcaacggtgtgatcttgggtcattgcaacctctgcctccccggttcaagcgattctcctgcctcagcctcctgagtagctgggattacaggcatgcgccaccacgcctggctaattttgtatttttagtggagatggggtttctccacgttggtcaggctggtctcagactcctgacctcaggtgatccgcctgcctcagcctcccaaagtgctgggattacaggcgtgggccaccacgcccagccagaatatgttctttaaaaacaggcacaattccatgaagtaaactcacattggaatggttaacaggtaaagtgatccagctggctgaaacaataggcattcaccggcatccaaagtacataaagatgagcactttgagactgcgccactgcactccagcctgggcgacagagtgagactctctcaccaaaaaaaaaacgtgagcactttcacctcaagagtcgctaggctcagatgctgaatgcaccctgaaggaactctaaagataccagcctctggagttttcacagccaacagcattcctaggtagactggttgagaacacggctagttggagggtaaaaagaatgacttgtgaatgctcactaggtctttgaatatgtaagtagacacgacttcaagccagttgctcagccttgaatatatcctagagtccatcaccttgggctgtgagtggcttgtgcggtcatctgtccatctctcagctgggcgagaaacttgatgtcctttctgagaggtggccctggctctgcctgggacctgctactcttctccaaagtgcattttttgtgggtgggttttcactgtgggtcctgggcaggtgagtgctggtgcccagtgttggggagcagggtagatagaggtgtggctgctacctggctgggcccggacttggcagccttccttcatggccctcagccacagcctgttccatagtttgttctcgatgtgtcagctttgtgttcccatgagaacaatgggctccttgaggacaggcattgcatcgtacctgtggtctcttccctaacgccccccagtacgcccagcatagacagcacagggtgcaagcagtcagatgagcagggctcccgttcccagcacgggacccagcccgttacctcgctgctatgtttcccacgtaaacggagcaatacccaattctaaagaactgttctgaggactaaatggaatagtgtaaaaaataaattgacatgaagtgaaggtcaattaaaatgcactagcttggccaggcgcagtgtggctcatgcctgtaatcccagcgctttgggaggccgaggagggaggatcgctggagcccaggagtttaagaccagccagacaacacagcaagatcccggctctagcaaaacattaataaaaattagccgggcgtggcggtgcccaccggtggtcccaccttctcgagaggctgaggcgggaggatcgctcgagtcctggacgtcgaggctgcaatgagccgatttcgcgccattgcgctccagcctgggcgacagcgcgagactccttctcaaatatagaattttaaaaaggccatctcgactgccgcgggggtcgcgtcctctccatccccgaccttggacgcaaccccgggc

SEQ ID NO: 163 - DNA, 2001 bp, Homo sapiens
agccttcccaggaggggtctgataccaaaggccaagttaagaaaatgtctgaggccgggcacagtggctcacgcctgtaatcccagcactttgggaagccaaggcaggtggatcacctgaggtcaggagttcgagaccagcctggccaatatggaaaaaccccgtctctactaaaaatacaaaaatcagctgggcatggtggtggacacctgtaatcccagctacttgggagactgaggcaggagaatggtttgaacccaggaggcagaagttgcagtgagccgacatcatgccattgcactcagcctgggcagcaagagcgaaactccatctcaaaaaaaagaaaaagaaagaaaaaaagagaaaacgtctgagaaccgacggcttgtatttaaaacgtgtgagtgggtggtggcaggagaggtagacagggagcagggccggcttggcggaagctttgctgtagagaggggcagagatggctcggtgggtggagggaggggcaaacggtgtttaagaagaaaaaacgacagcttctgcgtaggagaggggcctcctgtccatctccagggagaacatgggttctgtggccacaggtctccccagggcctggcatcaaggaggcgccaggcacgacctgcgggatgaaagcacgtggctgaagtcctggtggctgtgtctgggtgaggggtgtgagggcgtggcctgagcaggtccactctcaggggagaggggccgaggccaggaactgggtctgggtgtccacctggtgcctgggtgaggtcctgcaggcagcaggtctgggcttcagtggatgcagccagtaagggatgggctgatcagggaaaaccgtattttcaggggaggagcccaggaggcagggagctgggcggcggtggaggggcctggagggcgattgtccagatcaagcccctcgagtaaggcccgagaggctgtggcttcagaaatgaaggtcatgggcgactcgggccaatgccgcgtccttgacctgggcgaagtgagcggttgcactcacgagtgtggacggcccttccctacaggggaccttggattcgctgggggcggagtgggggctggggcccagagaacccgagtccggacgctcccgtccgtgaccctggccgggttctccgctcctcgcagcttcagtccgcaagtgtgagatgtcaggggacccctggacggcggggccctcgggctggcagcgcgcgccgcaggagtttcgattcggagggtctgggcgaaggccgagaactcgcgtttccagcgagttcccgggggcgccgctccgtcccagaggccggggcccctccggttgccgggcagagccgcgccgcgcgcggtcccattggcgcaggtgcgccgcgccccgcccaacaggtagccccgcccgacaggtagccccgcccgcgcctcgccattgggccgcggccgggtgtcccccgaggattggcccaggtggcggccccgcctcgccctcattggccgcaggtgccaccgcccgccccgggaccgcccgccccgcggattggccgctggctccacctcggtccgccatgttcggacaccgccccccgcccccgccggacggtcccgccccgcgcgccccccgctcccgccccgcgcgccgccggagttccgcggagttgaccgggtcggcgccgtcggtcctgagcgctgccttccgcgttccgccgcggccccacctggagcccccgccccgcgccatggccggagctcccggggcgcagcgctgacggcggcggggggagcgcgccatgcccagcaggaccggccccaagatggaagggagcggcggccgcgtccgcctcaaggcgcattacggggggtgagcggcggagagggcggggagcggcgcgggtgaggcagggagggcggggagggctcagccgtcggggctcctgcgcgagagggagagagggaaggggcggcgaggtcgctgcgggcccggggctgtcg

SEQ ID NO: 164 - DNA, 2001 bp, Homo sapiens
cacacactgacaatttggtccaaatgaattctaggaaatctgattaaactcagagggtaatgaaaatatttgtagtatttatattgtgcttagtatttgccaggtactgtcctgaatcatttgcatggtttaatctcttagtcctcccaacagtctattgggaggtattactaacagtctgtagggaagtactattactagccccatcccaggattacagacagagaaactgagacacatagcagtttcaaagcttgtctgaagttactcaattataatatgtcaggtttgtgattggaacctagcactataacttttgagatgatgtgtttactacagtgcattcctacttctgtaaatctcttctccttcctttctctgagcaaacacataaaactgggaggtaggtaggagaattgggatgacattcatttgtggaagtccatatactgtgtcatattcttcatcttcagcacttttaatctctaaaacatagtgcagtatttagcaggtcatcaataatttttgttgttgggtaaatggctgatcgaatgaattaacaaaaggactggatactccatttctgatcaccatagagataaccaaattacgtttctcaaactctgtcatgctgagggctgcagtgacaacattttaggactagaacacattacttggatttcacaatacaaagatgattgcagttgtccctggtaagctcagcccacagagcctttcaattcagcttggggtgagctgcacttcaggcatggggatataatagtgtgatgaaccattcttctgaaggttcttctgaaaagctgctggtcttgggatactgtgagagcccctctcattgctctttagaagtaatttaaatttttctgctacattgtctgctcagctcgtattctggtcatagaggaacctgaagccagagaaactagacaaaaaggtaagagaaaaccaagcaacaaatgcagtctcagctgttgctaacgttattcacaggagtgacaaactgaatgaatcttagggtgagagactggtctttttacctcatttacacttttccttggtctgcactgtagatcagcagaactcatttatcctaagaaatcgatgactaattttctgtgaagcagaatcaagaaatctggtctacattggttaaaaataagtacttggttcaccttgtgctgtgaagtccaagccaaataattgttgcaagttgttgaagcatttgagtgaagaaaaactgaccaagttcactatgactgcatgaaatgaaatgctgaactttttggggttgagggggttggaaggatggatggaggaagacagttgtttccagtctctgaaattcaattctgcagtaagtttctgtatcaccaagtgtcactttggcatacctcgtgcatatttatagtctaaggcttttcatataggattcaaacatagattaaagtagcagggaacttgagagctccagggaagaatatgatactttaattgcatttgatcattttattgaatttgcctaggaaaatcaattaaaacacaaaacttaatttttgtacttacagcagagattatacattccatcttgtccctgactatagatttttgccttttgcttttcattaaataatcaacttcaaagtactcagaaggccgatggttctattctgctgggtatgggcgaactggaacagggccttcattccacaaacattttacttgtttctttttcttcatctggcttctctatgggacatgttcaaattctatacataccaaagcagtgacttattagacgtatgtaatgttttaaaaagataatagtgtatttaaattgcaaatggaattactgcaataatatgaaatatgcatgattatatggaacagttgaagagttcccaagtgaatattaattgccttgtatccttccttgaggcaaaggcatataaaaataacatgaattcacaagtagttagagcc

SEQ ID NO: 165 - DNA, 2001 bp, Homo sapiens
ggagaaacaaacgagactgcctcccagtaagcaaaagtgatcacattttcacaaatgaaaatggtaaaaagaagtttaaattgttaaagaggaaagaatgaggagccaaagacaaagaaaaatcaaagctatcagagttgctgcagaggttttactaaacaaagaattttatgaagcagctgaagttcagtacaaaaagactccaccctacccagcttctgccgcgacttttccttgctctgaaaactccagcccacagaacagagctcctctaactaaaatgcctttagtgctccgttttaagcattcactcttttcttattttaaacgtcccactctcacactcagccaaaaggacttattgtaggctttcccagaaaaatttagtagtgtactctctccaacggtggatctagaagctccggatttacaaacatcccaattgtctttcaagtcgttttccaggtgcaaatccgtcagcccccttctcaaacttcaaaccagaacctcctcttcacgctcttcctgaggcttaacattccacctatttcccaaaccgtgcaataaacactacaagcaaagcaaatggcaaactcaccatcctcgtcttcttcctgggcttttattgagacaaattgccctaataaaacaataagaatgaggagaggtcttgcttccgcccagtttgccatcatgtctaaatattagacatgtgggttctcctgagagtgaaaagtagattctgaggttattgtagcaccatgaagtcagctgtgggctcttctttcagcaccagccccagggcagcctctgcaaaccccctttttcagcaccagctccagcacagtctgcgaaaacttttttcaagcgatgcagctttaaataggaagaatgctgcctccacttcttttcctgccccttttcagcttgttcaggctcataaccatccagtgtctgccaagcaacggtctgattgatggtaaacaaccaaatcagaacacgcgtctctgtgcctgaactttccctatttcatttaacttttaagcatagatggggcttaataacagaaacaaacatttgtttctcttttcttttattttttaagaaaggctgttagaagaaaagggttctattttcccctccctaacccttccccccagacacacaccctttaacttttcccctataagctgaaaaagacattccgaaggaaatccagccttttaccctagtttttttacatttagataattcaagactctcttctccaccttaaatctttaaacaatcacattttaaccctttcttttgactgaatagggagtattcttatccccttctcagattcggtttcttttcccatactctggaaggagattattagaatgttgcccaggtaaagtccaggttagaaaaccattttggataaatgaatttgtgatacattgtaatttgaattaagaaagcatgtcctttcctaatttctccacatctcttgttaaggggccacatcttcaggttcctcttgtcccagagagttttaccatgttgaaaaatagtctactaagcagtgcagaggaatgggaagtactctacctttggatgaaaaaaggcttgcttctgccttttgtgagtggttctgccactcaataattttgtaaatgtgcatatagtcctttatctctgtgcactccaattttctcatttgagaaaagggctttaccctgaccagtgtacagggtttagtgacactaaaatgagtaatgtattattaatgaagctccaaattgcaaagagctacataaaagcagatggtggtattattagctttaagccagctttgcagggaatatgatagccccagttgaacatgcattttaaggaaaattatcgatgaacttttgtgccaccctcacacttgccaagatcttaatgacctcagagagttctatacaatctaatgtatttgccaccttgagtctccctagaatcaggatggat

SEQ ID NO: 166 - DNA, 2001 bp, Homo sapiens
gaagtatacaagcgagggatctaggttgcgtgctccctataagaatctaatgcctgatgttctgaggtggaacagtttcaccctgaaaccatccctccccatcccctcagtccgtggaaaaattgtcttccatgaaactgatccctgaggccaaaaatgttggggactgctgttatacagagctgtaggagagaagaataatgcagagggaaaaggagtgcctgtctgttgagactttcagggggacctagggactggttttgttacagttgactgggaattgtcttgtttattttattttatttttcatttaaaaaatttttttgaaatggagtcttgctctgttgcccaggctggagagcagtggtgccatcttggctcactgcaacctctgcctcccagataaagtgaagctcctgcctcagcctcctgaatagctgggattacaggcatgcactaccacacctggctaatttttgtatttttagtagagatgtgtttcatcatgtcagccaggctggtcttgaactcctgacctcaagtgatccacctgcctcagcctcccaaaatactgagattacaggcatgagccaccgtgcctggccaaagtgtcttgttttatttttaagtctttgatcactctcatttacagtaagtgtttgaagagtacattctaaatcatttttctctcttcctttacaggaagtgctcaaatgttgagcacacacacacaaaaaaagaggaaacagttggataggaactcagaaatcatgtgactgatgactaagttaaatcttttctgcttactgaaaaggaagagtctgatgattagttactgatcctctttgcatttgtaaagctttggagatattgaatcatgttaccatttctgtttttttccaccctgttttcttccatatttactgaagctcagaagcagtattgggtctgcaactcatccgatgcaagtatttcatacacctactgtggtaagtaaaaccgcaaaacaaataattgtagcatcaactattttgagggtaagttttcacgagaaccgtacactgttgtggctggaacacacgaaacatcagtgtgttccagctgctgtggcggacgctgccagcaggaaaagcaatagctggcagctgccccatgagaatgttctcgaccactcacagagcttgtgagtctctaaactgtgttgtgcttgacctccaagtgcttctgttccctgcgtcatctttctctaccctgagcccagtttttggttctgcccatttttaataaaaccatctgcttctgagctttcacctcagccttgacacttactagttcttcttcttgagatggtagagcagtcccagctgcttcctggagttgatttctgctctaagctgtgactcttgagtgctcttggaatcatccccctcactggatgaaagcctggttctgtcactagctgtgtgaccttaagcaaagtactccagcctttttagcactgaagtgaaagtgattttatagtctacttgataagggtgttgtgaggtgaagttttttagcatagggtcacatagagacaataatactcttaatttttttttggagtcagccataccctaatactattcactggctgtgtcactgtcccctgccccagctgctcattagcaagtgtcctgggatctgaaggcacagtgtcaggcctgacagctgttttggttcggtactttccaccgtgctgcttaccatgggttggctgcccttttccacccactagcaatagctgggaggtttaaagtggtgcttcccaaaataccagttctttgagattcttttgggaaaataggatctatgacctaatcatctgggagagatttacagatgtactttgggttctgagaagttctgcaataaaggcacctgtttaaccacattttcaatttcatttgttgtttgtttgtttgtgttttagtctggagtgtatgttaactctcttgagaa

SEQ ID NO: 167 - DNA, 2001 bp, Homo sapiens
tggccgccattcccgtggcttccgtgggttgacagcagggattctgacaagatgaggttgggggtagggggtaggtatgggaatgtgagtggggtggcggtggagatgaggagacaggtgtgggtggggaaccctcagttccctcccagggtcctgaccctaatgccgggcggatgcctggaccttgatgcctgggccctgccctctcctgaccaaccctgccccggtcacataagctatgactacagattgccctgtgggttaagtggcagtgggacgggacggggggtggtccagctgagacatctgtcctggccgggtggtccagggctggcagcaggggtggtactggatcattttggggccatctacctggggtgtgtgcctcctcagtcctccctcagcatctcaccttgcctcctcacagagcctaaaaccactcccctcagtggatctcaagccgcccctcagaaccattaaatgaacctggcttctctcagatctgtgaggttcaagtcagtggtttcacatcgggccatccagggcttccctgggttgagagcaggggcaaaccggattctgccagaatgggggaggcgatgggagtggggcacatgagagagatgagggtggggttgggggcagtgagggtgaggtggcagaagggttggggctggtgctggggctggagggtggtggtcaggtgacggagtaggagtagggatgggggtggaggtgtagctgaggagatgggggttgggtggggaatgggaatgcgaatggggtgggagtggggattggagtgcagtcttgggggcccacagccctccctcttgggtgctgatcttgatgcctggcagagcctgtgccctgccctctcctaaccagtcctgccctggtcacagcaacctctgattccagagcttggggagaagaacctggtgtctctcaaggcttttggagcaggggtctgggggcagcccaggctgagacgtcttcccgcgggtgggccagggctggcagcaagggcggcgctggaatatttggggctctctatttggagtggcggggtcctcagtcctccctcagcctctcaccttgcctcctcacagagcctgggtcaggttccttcggccgaactctacgccgtccctcagaatggaaacctcgcttctctctgccccacaatgcgcaagtcagtggcgtcacatccgggcacccggatgtttcctggcgggaaccggatcctgcctgggtgggggtgggagtgggcgtgggggtgggggtgggggtgagggtggaatggctgggcggatgggcatcagggttgagggatggggtgggggatgtggggtgggggttggaatgacaatgaagagggggttgggtcacgggagtgagcattggagtgggggttgggtcacgggagtggggtcttagggaccctcagccctccttgagaatcctgagcatgatgtctggcagagcctggacgctgccctctcctaagcagcccttccctgatcacacgagctctgacttcagagtccccagtggtttaagcagcaagggggctgggggtggcccagcctgagaagtccaccaccagatggtccaggactggcatgaagggattatttggggtcctctatctggggtaagtgggtcctcagtcctccctcagcatgtcaccttgcctccttacagagcctgggcccgcttccctccaccgatctcaagccgcccctcagaccgaaaacctcgcttctctctaaccactaatgctcaagtcagtggcgtcacatctgggcacctgggcttccctgggttgagagcaggggtggaaccggattctgcctggcttgggctgggggtgagcgtgaagatgtttggatgggtggcgtaaagggagggattgggggtggtagggggagggatgccagtggtgccccggggcttaagtcgtcgtgttggtggtagtggggttgggcgtggggttgg

SEQ ID NO: 168 - DNA, 2001 bp, Homo sapiens
cagggcacagctggcaggcagagcctgagaccccaggagtgacaataggtgggtagggccgggtgctgtggggtctcctctgtttctgggggtagggcccttggtacacagccagggtgtgcactcaccttgactcctggcactgcctgggcctcctctgctgtgctgactttaggctgtgtgcctcacacccagttcgtcaccacctggttcctggagcacctgcaagaggaagtgagggagcccctcaggctaaagactgcaagtagggtcttgggcctgctagggctgacagctggggctggccagaatcccagagcctcatagttctggactgcatggccccctcagaatttaattagcaacttcacggcttctcagagagggccgagcctccaccactctgctggcctaaggcaaaacctcagagcaaccgccacatccctgagagcagtgggaggtagggcggtggcagccacctcccagcactctaccatgggggcagtcggggtagcctcacgtccgttctgatgcacatgtgactcctcagtcttatccccagcagggcctgggaatcttccctctgccaaccagaggcagctccccgtgctgacttgccatgggaccctcagaccaaggcctccgcctcccctacgccactcacccaggaataagatggctgccaccccgcctgagctgactggggagctctacgagggctgatacttgaggagcagggtgggaatctctgggaatctgccctctgaggtgggggttcccccagccctcctcagtgtcttcctatttattccctagagggccggcacctcctcccttcttctggggccatttccctgtctgggacctaccagtctcacggcagcgcagtacaggccggggtttcttctctctatggtgggaggtccctcagttcttcctcagggccctcaccctgaggactggccaggtctaagactcttccccccgctaacttgattcgcgcctcctcacaaatgccttcgatccgtcagattcccaagatggccgtcgggaaagcacatccgggaaccctgccgtggttgtccagggctgacgcagcgtcaatgtggggcggggcctcctctctgaggaggaaccaccccccacctctctgctttttttccatcccttcctccccacctcccagctgccctccaacccttcctgctcactccctaagccttgcggacctcttcactcccacctccccagtccttgacaccccactctcctttttccaggggtgtcagcagggagtggcttggtgcagtctgcgcctcccctccgtgttgggaggtcaccgtccttccacatctctcttgttttttggccagttctgggacttctttctctgcttaactgaagctgccctcctcagagacaacagcctccccttctgctgactctcaaaatggaagtcagaaaattgtacatctggggaccctgatggggtcctccagagctggcagcagggacaggggaggggcactaccagtcatgggtgttcccttccagccttcctgagaggctgtgcctggactcctgatactgcctgggatgcctgaccctgtgcatctgagtctttcacctccgaataagcccctcccatccctgagacctgcaaggtgcaagagagggagaatcacatccatccacagtgggatagggcctcctagccctaaggcaggcctccactgccctctgaaatggggtgggaatgcccctctgtctttctcaaggtatgactcctaaaagagcctatactccttccttctcctgtccagacagcaacatttgttccttcgacctctaaattccattccaagtttttccactttactgctgtgagtgaaattagtgtcatctctcccataccctgtgttaaaggcttccttattgttgcttcttttcctatcctagtccctgaaaaaaaccactgtcttttcagcaacccgtgaccct

SEQ ID NO: 169 - DNA, 2001 bp, Homo sapiens
gctcgtaatcatcctctgaacgcttctttttgagagaaaccacaagaaggtattcaaagaagtgctgccccccagcaaagttgggcaaagagtgctcctgggccctttctggttcctttaaagcttcccctgaattgtcctggggtggtcctgaaatcaagccagagcccatttagtataatctcattgatccattcctggagtggagtctgtgagaatccgggtgacctccacacctcaaggccgccttgccaatgctgagatacctggatactggccagaaccaccagtcactcaggatcagggccaaatgggcagtttccacacctgccctggtagacttgaaaccatggggagccaagcagaggaaagaaaccactgacacagaagccacggaaatgtgctggagaggccacatggaggccaaggccccagggtaccacagagactgcctaacctgtcgggtgcttctggcagtcctggaacaatatggcctgagagtttagttcatactaagtgaataagaacaggctcaaagggcatgtgactgtgaccacactagccccagagagggcatgaccagctcttaaaaaccagcagaggaagcctcctgacccagtcacatggggaaccatgtggaggccgagatataaaggaagaggaggaaggagcatttcaaggaagaaacagtcccgcctaagagggtcaccctaaaaacaaggaagtaggcagtccagtcctgacctgctcggagttgaagcagtcggcgttggaagagcctgaacacccggcctaccacttgtccttccatctctgggccttcaggacagagcggactcccctctcccctaacacagacagactggtgacagtaagcctgagaaaggggctgcttcggtctccagccacaactctgtgaagccaagccacgcgctgtcttccagagagggaatagaagtttccatcctgtgcacccagtggttgagcaagaaggatgtgacgggacccaggaaggcagcagaagccagcaccaacagagtcagcatcctggcactgcagcgcagatctcagctcctcccaagagccccagggtgtggcgagagaaaaggtcatgagcaaacacctctggctgaaagtagccggaaaggcctggctacttatgggctaagggttttgaatctgggctgcagattgtgcagagagttaatttacaaccttagaacagtggacaatgcttgatagtgtcagactgtgggcactggaaaccggctggtctcaggatagggatcctatacctccaacacttggattgctgacctttcacttctcaacttggtcaacggaagtcaaaaaccccagcaaggccacctgctgaattctagaacttccaggacttcccgacctagagcacatggccaaacactttctgcagccaggatgtcactgccctcccctgattaagataaatagacttggcctaataattatccaaatagattaattcttccaacaaaagaaagagttctggaagtcaatacccaatagccttctttcctgatggtgcagtagagagcaaaatccatctctaagcccagagttcgtgatattttggaaatagctacaatggtgaatttcaccccctggagagttctgtcactggcacttgttccagggttccagcagagtccctcttctcaaataggaagcactggctcagaagactgtccccagcataaggtaggggactgattgcaccagcagcctgagggtgattccatggcagtggaaaacatctgggtgatctccacccaacccccacgcctgctcagccttcctccgggtcaggccaggtcagctgttctgcccaagctacaggcacagctgctagcccactgctgtgtctggagactaggtaagagagaaggaatagcaaaaaaatcaccttggattctcacagaaacccctgaatgagtaaatacagggaaggcttttaccaggaag

SEQ ID NO: 170 - DNA, 2001 bp, Homo sapiens
aacatttcacaggggtttgggctgtgagaaacatcctgcctaaccacccgaccacaaggcagacaaaggcccaactaaagaaacatccctctcatatcttcctgggcaaaggtccaagggacaccatgatgacatcccactggaacaagggccaaaccgcctcatcatgagaacatctttatatcaatatcctgctaggcagcaacccatactgcccagacccctaccgcccatacctataaattgcccagtctgtaagcagtggtgggcactggcattaggctggttccccacttctgtaggttattatgctggacataaagcctgcgtttgctgtcgagccgccctctttctgtgtgtgtgtgtgtgtgtgtgtgtgtctttctttaacccttgccttcttttcaaaacctaacacgagtagacaaacgttctttctgaagagccagattgtaaatattattgtaggctttgcaggctatagctcttctgcccttgtagtaggaaagcagccacagtcatacttaaaggaatgagcgtggctgtgtccccataaagctttctttacggacactgaaattttaatttcacataattttcatgtgctaagaaaatatacatatatattttgtgagatggtgtgtcgctctcgtcaccctggctggagtgcaacggcgcgatctctgctcactgcaacctccgctacctgggttcaagcgattctccagcctcagcctcctgagtagctgcgattacaggcgcccgctaccacgcctggctaatttttgcatttttagtagagacggggtttcaccatgttggccaggctgctctcgaactcctgacctcaggtgatccgcccgcctcagcctcccaaagtgctggggctacaggcatgagccaccacgcccggccaagaaaatatagtttttttattttttccaactatttcaaaaggtaaaattcattcttagctcccaggttgtccaaaaacagacgcgcaggatggccaagcaccagagtttatcgtttgcccataataatcgggttgattcggccaaggtttccctcccccaacacggtgcgcactgggaagcgcgaggaccacgtcccttggtttaccactgcgcacctcacaaggcgcccagtgcatccgggtagcgggatgccggttggaccaacgcgagggtgggcggggtctaagagctaagcgttgggggcggggcgggacagtggggaagaggacgggtcgagtgctggcttccggcggaaaagcgcgcgagcaagatggccaccaccaagcgcgtcttgtacgtgggtgagcaggaggggttgctaggcggagtctgagtgaacgcgacccccaagggtcggggcgtggggtgggacgcatctctgaaccaggaggacggcgagctgctgtcaaggccgggtctctggcggtagtgctggcgatagctctggctgtgcttaaactccttccaaggtttcccactgtcctcaggaaaaagtgcagacttcttagcagtacgcacttctccagcttctttgctttgcttcccgattcgctgggtctcccgaaaaatgacccacttgcccccaaggttcaggttatttcacacctttgggcctctgcttttgcgactcccttgcagtgaacagccttgccacacacctcaggtgcgtgaacccgagacggaatgattattctgacttcctagcccgctgagctccagcgggccccgaggagcggtaaaggagtattatggcccttcggatcctctcgttcatttgttttacaaacattcagtgcagcgacggctcagccccagcccctgccataaaggaaggagttcccagactagccagaaaaaaagtggaattacctcagtagcacacttttgataagcgctgtagtgaggatggatcatgataatatgggcctggctgcgggacaaatgacccatccatctggagaagcgagggcaaattctggatgcg

SEQ ID NO: 171 - DNA, 2001 bp, Homo sapiens
actttcggacgagcacaccagctgtcagttataaatgataattaaggcatcttaattattaagcacagtgaaaatatgcagtgttcattttttataaaacttgtgtcctaaggtagaagaatcctcataccaaccaaatgctctttctctacctttgatcagcaatttggccttttttctctctgaggctgataaattgcagcaacctttgagatactagaccctccacccccaccccaggtcttcccccaggtctccttagttataaaggtagcagaatgtgatggttgaaagctgcatttgtaaccagcttaccttcgcaacaatagcaagagtaacaaagaatccatcctaataaaagctctgtgcacagtcagcttaagcaacgtaaagtgttcctttattctgcttagtagcttctcccccagaggttaaccattttaaagctggcactcagcctgagaacaggaacaaagcattactagaaagcagggatcttcctccaggatcagcatctctgctttccgtaattcttaagaaatatagaaactcaatgaaaaggaagtgatacctttggatgggcacattccagcacaggaaaatcgctgtcttgtcggctgctgtgcagaatgagttgcttgggcaggtattcctctattctcagctcccgggtctgccttgtagccgcagccagcctagggagcatttacggcatgtacgtggtgttcatccacaataaaaggagactaaccagctcagcttggcgctgctcgcctggaggcatcagatccttggaccctccccatctgattggctgctacccggtctagccgaactcctctgccttaaccttctgctgcgttcaatacattttccctcgggatacagtcagagaagagctggttagggaatcagtggcaggttaaattgagctggctttaaaaatcagtgacatgattttctatgaagatgtctaatgtttaattttccaaagttctacgtaatccacaaggagagacgtaataaaatggatatgatttaacaggagcggcttcatgattcctccctgaaaatacctggagtttaatgcatgcctaacaccctactgaagtgatcaggtggctttttgcagcactgcgtgtctgtataatcaaccttaaatcaaaacagatggggatgacagaatatattaagaggcttattaaaagtgccaggagatgcagttgaataaggctgctgaagccatctcaagaaagtgttataaatcatttacacaaaatgctcttaagacagttatggttgattttagatagctctgataataaaaacataaagaccaatgacagacaaaaaggtaaagcctgacttagcatctctagaagacaagtaacagaaagaaaaatcaaatcttttactctacaaaagtagtgaaatgagaaaagaacaaaagtacaccttcatcatctcctgtccatgtactggattcaatttggtcaaggacctagaacagggtattgaaaagtccccaatggggttttggtttgttttgttctttctgtttttttttgttttttgttttttgttgttgtttctttgttttgagaccgagttttgctctgttgcccaggctggagtgcagtggcgtgatctccacgcactaccccctccacctcccaggttcaagcagttctcctgcctcagcctcccgagcagctgggatcacaggcacctgccaccacacccagctaatttttgtatttttagtagagatggggtttcactatgttggccaggctggtctcgaactcctgacctcaggtgatccgcctgcctcaacctcccaaaatgctgggattacaggcgtgagccaccacacccagacagttttgttttgttttgttttgtttttaaaaatgagaggcatcaaatggaagatagtgaattgaccacatacatttaaccctttctccctcacaaagccccactaaaatgacaataaag

SEQ ID NO: 172 - DNA, 2001 bp, Homo sapiens
cctagagtaagggacgcattttacactgctatccaaaactcatcatagaaacatacacacacaaaaccaaagcacacatatacaactgagcaaatatttcatgacataacactttctcttactaagggtgacgcgctgaaattttgtattctgtcctatttcattttttaaaaatggtaaccatgacctgctaaattgatttcattgtccactaataaattatgacctcagtttcaaaaagattgctttaggtaaccaatcatcttctgagatttatacagattgctcataattctctcctattttttaaaaacatgctgcagtgaactgctttacactcattttatgactacttctgagaccaagatcccggattatgtaattgttatttacttaaaattctggtaaaatgtagccattatactggaaaactaaattttaatcttggatctgtcaccaccatgatatataaactttgggcaagtccctgcacctctctggacctcaatctccccatcagcaacctgctgatcctactcccaggagtgtgctctaagttgaaagtagatgccccaccccctgagtcagcgccggcaggacttctcaccaagcccttctcccccttttccgctccctgttcctggttcctaggaagcagcccaaggagaagggaaaaggcaggtctgggcaggagggagcaatgaagggcggggcagagggagggcaggagggaggccggccccctagtaggaaatgagacacagtagaaataacactttataagcctcttcctcctcccatctcctggcctccttccatcctcctctgcccagactccgcccctcccagacggtcctcacttctcttttccctagactgcagccagcggagcccgcagccggcccgagccaggaacccaggtccggagcctcaacttcaggatgttgacaacattgctgccgatactgctgctgtctggctgggccttttgtagccaagacgcctcagatggtgagtcgggggcacatctcctgcctcaggatggttctggagaatctcagtctatctgggcacatggcaagaccacaggagagcttatctcacagcatctgtgtctgcagctggctagatctctctacagggcaggcagagtcttggggactggttcgtgtcccaaagccaaggtgagttagtacatttaagcccctgaaaagggggagatgaaagaggctaggggaaacaggatgactggaaacatgagaaagaaaccagcagagagggtaggagaatcagccccagggagaggggagaaaggggaactgagggtgatggtagataggggtacatctaggggagacgggaagaggctcagaagagaagagaaatggagggaatgggaagaccctgggaaaactgatggaagaagtgggggaagagtggggcagagagaggttaggggaggctagggaaaatggaaggagactggtcgcagctggtggaactggggagaaagagatgctgtgcctaatagaacttatgggcgatcaggctactgaagtggccctgtttaagcagaaaagggagttattaccctccattataattgcacaggggcctcctttcccctctctcacaatccccgtaacttcagtctccccctcagagaggcagcaaataataaccagtattcaatgagtgctcactatggttaatacatgtattgacccatttaacttgcacaaacccctaaaggtgggtaatattattactatctccattttatgaggaggaaactgggtcacagagtagttaaggaccatgtctagggttatccataaatatacttattcacatctgcagatacaaagcacaacttctcaaatgcaaacacagacaggacccactcacacacacagatttacaaccccggactcatccaaatgtgctctgggcatcaactctgtgccagcctcttttctgggtgtaggaagcagagattac

SEQ ID NO: 173 - DNA, 2001 bp, Homo sapiens
cccgtccctggggtccttgacccaggctcttggggctagcctatcttctgaggagcacaaggtccctgggggctcagggaagagaaattggagaaagggggaggaagcccccaagatggatcacccattgcctggtttcgcaggagactgtccgccttcagttctccagcagctcggggatcatggcccactgaacccccaagcgctttcacccgaacccaaggaggacgaccaggaaagacgggaactcgcgtagacacgcccggaagcccttgtcatgtaaatagctgtcggggactggtgtattgtcgccgccccagccggcgggacctggggcgaatccacacccattgtctgctgcccaaggggcctccggctggggggcgcggctgcggagttcaaaaggggtatgagcaggaggggtgtacttttagttcattaagttttaattacaggagtgctacaagaacacattcttcaggtttaaaaagatattaaaatattacataagagacctcccctccctggcccacctccagcctcttaaaaatttagtgtgtcgccttttagacactttctcaaagcttcacttatttaacaggcacttaaggagcacctacctgtgccagaaactctccaaatattaactcaacctgacaccgactcagtgtggccgaatattactctccccattttacagagcgggcagctggtcaaggaagtcgcttgttgaaagtcacacagtggtggagcctgtgtgccaacccaggaccctggggagctgcctccccctctcccacgtagtcctgattctttaagtgtccacatattcctgtaatgcctggagtttcagtaattagcagggacttagtgtgttcagagaaaaaaaaagcttttaaaaattattgttactgtgtttgtaacagtttggatagagaaggaaaagctggaatttgggaagtgaaggtggcctcggggtagaacttacctagaccagagcgaattcatcctgaagaactcagagaaagccggtgcaggaagtgggttcccgctctccctgcacaggcacagtgatgctgccagagctctcccagaaagaccaggaggcttgttctggagaagtcaagcccagggatgtggctcaggctggtccaagctctttggaggagtccaagcgtgcccagcccagagggaggttcagaggcactgaccgtcttctgtttgggaggagaagctcactcttggagccacagccagcactaggtcaggacccaggccccggcccaggagtggggcaatacccagcgtctaccccagatggcaccctgctgtgaactgggcgccctcagcccctgccttgaggaaggggcaataccaccagcgtgtcttttatcagggaagatattgctgcagtttggccgctgcaacttaagagaaaagctaaggggtcccccagcatcccttggggtgccactgcaaatactggctgggcctggagatgacctgggtcccattcacttcctagggtgaaggaggtcatcattaccacccctgctttcagccatttcttcattcattcaatcaacaaactggctgagctgcaaccctgagccggggaattcagccactccagacacagcccctgccctccgggaagtctcgggagacctggctagtctggctgggagaagtcacacgttgattgtcttggaagtgagatggcatttacacaatggaggctgcactgccagcaggcaaaaataaccagttaattcagtggcttaaagaaaccaaacctacccacaacgcttgacctcccattgatccatctgcgacaccggcagtggctaccatttattgagtgctgatggtgtcacctgggattgacttagtggtctctggcgctagttccgaagttgattctgtctggagagcttaatgcagtgttcagacctcagggtccgaacctgagggtcacccaaagatgagtgggacatagct

SEQ ID NO: 174 - DNA, 2001 bp, Homo sapiens
acacacacacacacccagcacacacctccccacaatcctcagctgtggggggcggttgtaccaaaggcttcaagaagccactgggagcacagagcactcccaggctctgctgtccccgcccagctggcctctatccaggtagggacactggccttccccatgtgatcagtggcagcaggtgtggctgtcgggggccctagccttatctcctaggatcaactctgaaacgacaaccaactgcactgctaccctggccgcagaggtacagatgccactgtttgtgcctttcagtgcagtctctggtctacagtgacaaaaggactcaagaggaggatgagaaaagttgccgtgtaacttttgcagggatgagcccaacagaagcccttcacggtctccctgacccctgccagcaaaaggagcactcctttagtccacctttagtccacctaagacctctcaagtagctgcatggctacacccccgttcaatgtgacaaaaccaccacaaatggccaagggtatgacaattgctagagaactacaacaggacctgtctctcacaacaaacccctctcctctccaaactaacaagagactgcttgccctcacagtgacctgatcttcctggagagaagcagcagcaagcttctgttcacaattacagggcaaggaatgtcgacgagggtgagaatcattcctccccaggatcgacaaaaggcacagtggaattctgcagagtccaggaagtttggagttgctaagaaatttgctgctgcagacacaagccacatagggccttaccagggtcaaggcatgaatctgtgggactgaaggtgagtgacagaagagagacacctgtgtccaggacacgctgcacaggaatccctgggactctctctctgtcatgttcctggttatcaggacggctcaccagctttctgctgaggccagctggacccaggggagcagcggggaaccagaaggagcaagggcatggccatcgggacagactactcacaagacctggctgagctgatggaactgctccagagcctggcgacaccagcactgttgcttgtcactgctgcaccctgcacacagcgggctctgcaggagccggtagtaccggcgacgggcataccggctgtccagctccccttccagttccgggtctgtgcccccttcccccttcctcttactctgcatatagactctcaagaagcacccatacagacgctggatcatctcttggaaggttctgggggtgctaaagaacaagactccgcgcaacatagtgtggacttcttctcgcagcccctgagcaccagtgcccatcagcaagcccaggcgagtccatttctccagcagctctaggctacgcaggtagggatccaggcggctctccagcaggccaaaagcgtcaaggagtagcaaaaggcactggggctcatccgcagagttctcgcattgggagatggcattccagaactcaggggagatgttggcctgcagatcgttctgcagcacctccacgaaccactcctccaggaccgagtgtagcccgtggcccctcagaacctccaccgccgcccggagctcctcttcctttggcgggactgcaccgctggtccgggaagacacctggggtgcagaaaaccgtgaggcgaggggaacacaacatagaggtggggagaggcggggaagggaagaagctgagggggaggctgcaaaaactccgctgccccctgtccgtgctgcccggacgttgggccgaagctatggaagggctgtcagcggagctgaacgaaacgaaacggaaaggtggtggaaaatggcaggacaggacagaggcggatgatggacagagccgtatgaacccgagggtagcggccccaaagcgcgtacggtcccggaaactccgcgcggggccgcccctctcttcccaggcctcaccagccccagcgcagccggcggcaccaggccggtgctcacggtgttccag

SEQ ID NO: 175 - DNA, 2001 bp, Homo sapiens
cgaggctgtgtccaggttcagtggggaaaaagtgccttgatttgaccattatatcgtgtactcagggcctagtttatttcccacaagcagctatttaaaacatgtctcatccaaggcagggtcggggtggggaacctcagaggatcaatgatgcagcctgtattgtcaccagggctcatcccaaagcaaatcctgctgaagttctcctctgaacaacccttatcactcatgctaatatgttggatcctgcaagtcacaaatacgaaggtatgagcattggccgaatcccaacagggaaacttcaccgggaaggtgaagacccctttccattaagataacaaacagaacctagaaggcccgtagcatggtggcttagaacatgtgggccttggatgggatcctgccaaggggtgtgtgacctctccgaagcctccagggatgatactactccctggggtgcttatgccaaccacgttagagacaatggtttctgtacccattgcctggggctgccataataaagtgccacacactgagtggcttaaaacaacagaaacctattgtctcacacttccgggggccagaagtttgaaacccaggtgtgttaggatcctgctccctctgaaggctccagggaagagtgtcctctgctccctccgaaggctccagggaagggtctgtcctcttaggcttctggtggcttgcaggtgcagccctccaatcctcctccccaagcggccttctgcctataaggacacgagtcatactggatgaggggcccactaattgatggcttctgtaaagtccccatctccaaataaggtcacattgtgaggtactgggagttaggactccaacatagcttctctggtggacacaattcaactcctaataacgtccacacaaccccaagcagggcctggcaccctgtgtgctctctggagagcggctgagtcaggctctggcagtgtctaggccatcggtgactgcagcccctggacggcatcgcccaccacaggccctggaggctgcccccacggccccctgacagggtctctgctggtctgggggtccctgactaggggagcggcaccaggaggggagagactcgcgctccgggctcagcgtagccgccccgagcaggaccgggattctcactaagcgggcgccgtcctacgacccccgcgcgctttcaggaccactcgggcacgtggcaggtcgcttgcacgcccgcggactatccctgtgacaggaaaaggtacgggccatttggcaaactaaggcacagagcctcaggcggaagctgggaaggcgccgcccggcttgtaccggccgaagggccatccgggtcaggcgcacagggcagcggcgctgccggaggaccagggccggcgtgccggcgtccagcgaggatgcgcagactgcctcaggcccggcgccgccgcacagggcatgcgccgacccggtcgggcgggaacaccccgcccctcccgggctccgccccagctccgcccccgcgcgccccggccccgcccccgcgcgctctcttgcttttctcaggtcctcggctccgccccgctctagaccccgccccacgccgccatccccgtgcccctcggccccgcccccgcgccccggatatgctgggacagcccgcgcccctagaacgctttgcgtcccgacgcccgcaggtcctcgcggtgcgcaccgtttgcgacttggtgagtgtctgggtcgcctcgctcccggaagagtgcggagctctccctcgggacggtggcagcctcgagtggtcctgcaggcgccctcacttcgccgtcgggtgtggggccgccctgacccccacccatcccgggcgagctccaggtgcgccccaagtgcctcccaggtgttgcccagcctttccccgggcctggggttcctggactaggctgcgctgcagtgactgtggactggcgtgtggcgggggtcgtggcagcccctgccttacctctaggtgcc

SEQ ID NO: 176 - DNA, 2001 bp, Homo sapiens
gattctgatgctacaaatgatagatatcgttttagcattttcttacgggtcctagcgattctattcatttttctttcagtctctttctctgacttgttcacattgaacaatttccttttgggataggttgctatttctgttttcgcaggtggtttacctgtcttcccagccagtcacagtggtccttgtccccatggtgggtccggggcaagagagggccctgggttgggggtggggttcagttgaagatggggtgagttttgaggggagcactacttgagtcccagaggcataggaaacagcagagggaggtgggattcccttatcctcaatgaggatgggcatggagggtttggggcgtggcgctgggaacggcagccctccccagcccacagccgcgcatgctccctgggctcccgcctcagtgcgcatgttcactgggcgccttctgcccggccccttcgcccacgtgaagaacgccagggagctgtgaggcagtgctgtgtggttcctgccgtccggactctttttcctctactgagattcatctggtaggtgtgcaggccagtcatcccgggggctgaagtgtgagtgagggtggagagggcctcgggtgggtcaggcgggtcccgcttcctggtctgtggcctccgagggagaagggccacgaggtcgtcctccttcccttcacaggctgcgaggccaccggcggcttcgtggtcgtgaaggggcctggacggggaggaaggtgggccgtggaggggaggcggtcaggggctcaggtgaagacggggtgagtgctgttggggggatggaagtcccgaggtgccgggaacccccgacgacacagggcagattccctgaatggggtccccggcgggggcgaggcgggcggtgaagaaggggcctggcacctgggaaggctgcggcctggtgagcgcccccgcagcggtgtggagtgcggagcacccgagtgagaagcactgcaaggtctcacctccgccatggaaggtccgaaaacagtgggaaggagtgggcgaggcagtgcggtccaaccaaacttgttgtgagggggggtgaatggctctaggaagtgggagtgtgcccaaagcagcaatcacgagaattgtgattcactagggttttcgtggggagtgcacttgtgaaactaaacctcatcagaaatgacctctgtctgcggggcgcagtggcgctcgcctacgtagtcccagttactggggacactgaggtgggaggatcccttgagcgggaggtggaggctgcagtgagctgtgatcacgccgctgcactccagcctgagcaacacagcgagaccgcgtgtccaaaagaaatttagaaaaaaatgtcctctgccttttgccacacgccttaagatgattgctctgccagcctggccagcagaagtggctttgtaggcactcagacagcgtacacacgtatgcttaactctgggacttatcttgagagtattttcaaaagtaaaacggcaagtttacatttatccatggaagtgatcgaatatagcagccctgtggagcgcacgttcccaatcacggttgtctgttttcagtgtgaaatatgagttggcgaggaagatcgacctatcggcctagaccaagacgctacgtagagcctcctgaaatgattgggcctatgcgggtgagtgcttaaacgttaattcgatgttttctattagtagaaattaatttttgtgatagcgtcgttgcattagtgtggaaatgctgataaaggtctttcctgctcataaaaaatgatgatggcatctcatgaaggaaacattgattctggaggattttttttttcctctcgtgttcttcagcttttgcccatgacttctttctccggctttgtttgttaatgacagattgtacacatgtattccaacacagagtacaatagcctccaaagtcctcgtgcgtcacttttctcacagtaacctccctgtgggtggagtaacct

SEQ ID NO: 177 - DNA, 2001 bp, Homo sapiens
taaatttgatcaaattctctctcgtgcagttcaaagacatctgccagctgggtgcagtccacaccgaaacgtacatcgatacctggcaaagatagtttgggggcgcaaaatatagacaggaggatgcctcaagacatggaacagaaaaactcttcctaacttaaaccgtgcagaccagcccaagtctgggcacaaagccggctttatgtgaatatccgtgggacgatcaagaacaatctccacgtgggttacatccccacgcccccaccctttccactttgggacgcgccggccgctgggcggggtttcgcagaggggcgggctcgctacctcgctcgcgcaggcactgcagattctcccaggccagtggatcccgagccaactcggccgggcgctggaggcaggtggcggtaagttgagtgctctgatccagggtttcgctcagagcggcggcgaaggagaaattcccctccccaaccaacaggaggcgccgaggggccatggcctccacggactcccggctcgcgttctctgtggcgctcgttttacgtcacttccttcacggatctctctccgccccgtcttctcccgtcactagttgatgacgcacccatggcgcctggttgtctagacgagccggtgtaccgactccccggtggatgataactatgagtgcaaaagcaagacgcgcactttcctgtcacctcgccctttcaccccctcgggaagatcaaagtgtctttagcccaagcatggccttgagcgacttcatcacacctgcatgaggtcacaagcagtcactagcgcaggggggtgaggcgagttctgggtgaaagaaacggggcggggctgaggccaaggaagaggttggactgtgtctcgcatgctcctgacgcagaaggttttgaaccttttttcacctcgtctgaaatggctgcctcccagtgtctctgctgctcaaaatttctcttccaggtgcttaaatctcctatcttctattcgttcaagaaagagtattgagtgtctattgcgtgccaggccactgtgagggtggttacgatatgtggatgaacaaaatgtggcctatgatttcaatgagcctgcagtctggtggggaatgcacagagcaaaacaggcaactctgatatggcagggtgtgtgctctgggagcacacagaagaacgttaccaagtttgggggcgaagagggtcagagagtccttggatgaagtaaacccgagttgaaagctaacagggtaaaagtgcatcttaactgagtcaagaggcgtgaagaggcagaaggaacaatatatgcaaaagcttgatggagaaacagaatagttgagtttggctgaaacacaattggagggagagtaacagatgagactgaagagttaggcaggggccaggtcatagggagcgttgtaagcctttaaaaaaaatcaacttttattttacaaatgggggtacaggtacaggtttgttacgtggatatattgcacccaggtaggttgcaggtttgctacatgggtatattgcacctaatagtttttcaacccacaccacccgccctccccactctagttagtagcctggagtgtgtgttgtttccatgtttaggtccatgtttgctcaatgtttagctcccacttataagtgagaatgtcagaggtatttataccagagcaactccgtcttgaataggggctaggtaaataaggctaagacctgggctgcattcccagtaagttaaggcattcttagtcacaggatgagatagctcagcacaagatacaggtcataaagaccttgctgataaaacgggttgcagtaaagaagccagctaaaatccaccaaaaccgagatggtgatgagagtgatctctggtcatcctcattgctacactcccaccagcgccatgacagtttacaaatgccatggcaaagtcaggaagttaccgtatatggtctaaaaagtggaggtatgaattaatccacccctt

SEQ ID NO: 178 - DNA, 2001 bp, Homo sapiens
tgggatgcaagacttatgagggcaaagattttgtaaaattcactatgtatcccaacgtttactgaccaatagtcataagatgttttcttagttcattcactcacctgcttttctaataattatttcatatcgtctgttttctcaaattctgttcttttacatcttcacattctgctaaattaccgtgcttatttcactgatgaagttgaagtgtttagaatggaactccataacctcctaccatgatatttttccacttaccagcatctgtaccaacactctgcctttgctgtttctgtaaatgaactattcatgcctctctttgcagccagtctttgtttgcatgttcatcctgtagatcccatcccctttcactttcacaaggagccattcttccctttctgtcctatatcatcaatttccccatgcctgctggatccctcctgtgagcttacatacatactgttatttcttccatcttgaaaaacaaacaaaaaacctctcttgaccacacttcccctgccagctaccatcatatttccctcttttcctttggagctaaactggttggcaaagtttattactttcttcaattcctgttgttctctctgaaacccactctaaacagaagtttgatctttccacactaccgaaactatttttataaaagtcactgataatctctgctttgctaagtccaaagatcaattctctgttcttattttacctgacaaattcaataatttgacagatttatcactctgtctttctgaatatatctttttcatatagattccaggacaccatatgtttctcggattttcctatgctttcctgatcactcttcctcagtctcctttgatgccttctctctctctctttttttcaactacttacctaacactggagtggtccaaggctcattccttggacttctcagtctattctcgccacttctgtcatgtcagtcagtcacacagctttaaataccatctgtatcgaataatttctaactttttgtttctagcctaaaattccctcttgcattcctgactcttaaacacaagtgcatctttgacattcctatctggatgtttaacagacatctcaagcttaacatgcctaacctgctcctgatcctttccccaggctgggccctccagtgaacttctgcatccagttggcagctccagacttatagttgtcaacaaaaacactggaatcatcctgcattcctctctttctttgacacaccataatcaatctgtcaggaaatcttgttgcctttactttcaaaatatattcagaatctgacctttgccctgcacctccagctctaccaccatcatgcaagccactatcatctccacctgattttctataatagcttgttaattgtctctctgcctctgtcctttcctcctcttccgcctttgtccattgtcaaaagcatggcctggatgattcttttgaaatgtgagtcacatcacatcactcttctcttcacatttctgtggtagcccccaattcactcagtcatggcctacaatgctgtgccctctccgtgacctctctgatctcatttttatttattttgtctttgcctaactccactccagccacactggcctcttggatattactcagacaagagaggcagcactccacctcagggtgtttgtactagttgttccccctgtccaaaattatcctccccaaaatactgacatggaggacactctcacctcctaaaaatctcttctcaattagaagttggatcttgtaccctcaggactctcagttcctcttatcctactttgattttcttttccacagcatttatcaccttctagtatactatatcattaaagaatttattatgcttattgtctcttgtcatcttagtagcacataagagccgtgagagtaaaaatattaatatgttttcatttcattcgctggaatatccaataattgctgcccacacttactatgtgcaag

SEQ ID NO: 179 - DNA, 2001 bp, Homo sapiens
cagcccacccgcccgaggcggcggcgacaggacactcgcgccaaccgagaccccgcccccaggggcccgcccccccagggctccgccgccaaccctgcgtccccgacgcccgtcaccgtatgtccccgcccacccccggcctctgccccgcccgcgcagtccagcctggtgtgggctccctgcaaccccaccccctaagccgggtcccgcccccacatggccccgcccttatctcttctcagaccccttccatgggttccaggcaccttctcgccagggtggcgaggagaaacaaaacacatctgtcacacagccacatggcctcctcttacttggacagacaagcgtccccagcaaagcttcccgtcctctcgataggtgactcgtcctaaatcagctctgaattcccgcaaagacttctggaagctgtgctggggttgaaactggtagaccaaaaccagaatcaagtctgttgggaggggcctttcccctgggacgggaattccagtggtaatgtcttcctggggcttctccggaaggtccaatttccactccaccctgctcattctttcaccatgtatttctcacaaacactttgagctggattgaaggggaaacatttgataatgcaatcattaatggagtatctgggtattcggcaggaagtcagtatatctttcacactactgtttagacttattcaaaagaaatttaaaagaaacatgaaaaagcctcatctactgttcaacactacttcttaaaactatttttcgaagttttttcctagtgccaggcacagagcagtttcttcataactgttcattgcatgaaaacaccaagtgtgtatttctgacaactcacttgaggggaagggtaagaatcagttctgtaagcacaactctgagctcgcacagagccccacgatgggggcggggagctgaagactcctattctaaggtctccaggagcaaggccaaaaggaggttgtcagcagtatgcacgccgtgaaggaattccagacttaagcaacagactgagaagcatgaaaaaatgttttgtcaactaccatatctgccagtgagaacctgtcagagtaaaaaagaatctgaggctggccagtccacccggacaccttggcctggatcatttcttcttccataacaccagattgatgagctgggtacatcaacctgaggcataagaaagctctttcccaagcaccgttgaaaggcgcttcacagtgcttcaacctgtagcttcagcttcatcaatctaccagtgctgcacagctgtctttggtattgaagatagccaagcttcagcttctttttcttcattcaaaaaggacaggtcctcaatccaactttttcttccaattcttctcagccaaccatctcaccacttccttacctatcacaaggccaccttcccctgtgcactgagggcccttgacctctttaaggcgcccagtgtccacttcctggacacaccttcccttacgaagttgacagtatctcctaagaattcagtgtacacaatggtgccggaggaaatggcataacagaaggcctaagagacggatggattcatgaaactttggcttcacaaaagatcaagtttttcaacacacaggagattaagtttaccacatgattcattcaacactagtaattttatttcaaaatataaatatttcacattaataaagaagttacatcagtaggttgtcaaagcaagttagctaagaaacagcatttgtgtgagaacagagggtatttattgtattcgaatattcttttttacttttgaattgcctccttcacctgagaacccagggatgatacattttcaaagagttatgtaagcaattacaactctatatcaaatcttagttcaaaatcaaattgtactggaaatacacttcagttacctaaataagtggcactgcaaagggcaaagaagggttttctttgggatggctacaatgagagttacatctgc

SEQ ID NO: 180 - DNA, 2001 bp, Homo sapiens
gattcctctactcaagtactttaagaaaagttcctgatttttccccttgttaagcccgagaaggaggtgggagggagaatgcctcccctctgctattccttggctccaggtttctctttaagaaatgcaatcagaccacatactcctcagcatctgagaacacttcctctggagtccagttctcctttccaggcagctgagggaagagagactttaaaactggcagagggcatcaggggtgaaaggcaaccctgagagtaggaaaaggccagggagacaggccgattccagcaggggagactggcccatctggagcgccagcacatgcctggggatttcctggcctcacctgcagacaccctcccctccctgagggttgggatgctcagagatcacagggactctcaacccactgctgggagtggcccctctcagacaggcatttttattatcaaatgagcaatgagagaggggagaggtgaggaactctatgagaaacatgcagtcttttatcacgtcatcagttttcagtgagaaaatccagaccttcttacttttacagagatagaattcagttctcagggacttgtgaagatgcagcatcatcaaatcattgtgtgtgtgtgtgtgtgtgtgtgttgtgttttccttatcatccaccttagaaaatattctccacattcccatgttaaaatattttttcctaaaatccatccatccacatgccaacatcctaaagacagtaatataggttcattgcagaggatttggaaaatacagaattgtgaccagaacaaactaaactgcatccataattttatcatcgaatttgatcaacattttggtgaatagccaccagtctgttttctatctttttagggaatataagttccaagaggcagggatgtcattcacttctgtgactcagtgctctgaatcaggcccagtgaagactgacgtgcagcctgtattggcagaatgaatgaataaatactacacacgcatattttgaattgaaataagttgtcagggtgcagcagagaataacagaaatagaattgcttggatgtgctgacctagatccaagctgctactggtaacgtactgtatctccttgaataagtcacttcacctctctgggcctgagctgcttcttcagtaaaataatccttgtagctaagatttgttgagcgctcactatgcacactgccatgagcccctatatgtgtatttctcatcatagctctatgagttacatacctgccatctccattttatgcatgagaaaataggcatggagaagttaagtggtttgcctaaagtcacacagcaaatttgtggagagctgggcttgaactcaggcatctgggcttttactctctccagtggaatgcctgggctgtgggatgagatgacttcacaggcctctcccagctccagagttgagagagagagaggagtggtgcagggaggtagtagggatgattatgctactggccaagttcaagaaagtcaattgtagaacaaggtccagctgattattacagagagcaaagttcttaactagaggacttcctcctggaatgtggagagtccacacgggcactagcagagcataatgaaagaccaggaattctctgctgttcaatcagtgctccatcactcagaggcagggctgcaagagctcctagtgtgtccccttcgccttcagggcctccctgtacagccacccacaggacagtgcaccagggtcaggcagggaagtaaataaaaaaaggaatctgataattcctgacagtgagcagcagctgatgcttttaatgggggaggaagactgggaatctcgcttgatgatgacctaggacttcagtcagcaggacactggcccctttcctccccctcccaggctgaaaccttgtgttttgccttatgtccatggtaaccaagtctcctttttggtgaggaggaatgggcttcagtaaacagatctatttgtgtgat

SEQ ID NO: 181 - DNA, 2001 bp, Homo sapiens
tgtagcagttgccctgcccgttcttgcaggtgaccttttcctggaaacagacattctggacatctaccaggggctcgtgcacaaaggtgttcactggtttgcaccgcccctgtgtcatattccggcgcctcatcatttggttacagtaggtggagctgctgctgggggaactgtctgagtccatatgctgccgctggaatttcttggcccgggattccttgcccagggaaggctggacccagcccagcaccagcagtatcaggacaagcagaaggagccggacaagagacttctccagagccatggtggcctcactttcccagaaaagcctaattgagaaaaggagagagaaggaaaagagccctcttagaaaaagaactccagctcccacctatggcttgcctggtttacactttctgtttcaggtcagccagaaagacgtccatcctacttcctgaggtttttcgtgtaatgagacactcacgtgtacccttttccacgagcgttttattcccactctccaaagcgaggtcttcctcccctctaggtggtgacctacaaagtgacctaaaatcctgtcagttcctatctttgaatactggagtttctttgatccaaaatacatttgtacccagtgctcaaatggctcatcgaaacatcagtttgaataaatcatgaaccaaattgtatccccgacatccagggccatgggtgacagagccttgttgtcttggagcataagggaactcaggccagtgcctgacactggcatcataaaagattccattgtattccagagagaaacgctggcctcccccaatttggaggctccttcttctccagtcccatggatgctctgcagcctgcacgttccaccctgacccctctgcccccagctgatgtttgcctgggccacattttttcagcgctagcatcacccaatctttctcagtgctgtggactggccctagctccctcttctgcaaccctgaagcagaagcggcacctgagaacacctcttgggctgcttacctggatctgcagcttggtgtctgagagagatggaaggccagtttctgcaatcactcagctcccggggtgtggaccttatacagattacaaaggggtttggcttccaagaggaaaagggtgggaggagcatctggagctcagttcaaataaaatcaaggacgtgaggaaggagtggtgaatcacccaggccgccgtctttctagggctgctgaagaaagagcaaatctttgcctacgttagtctttcttcctatgtgaaccacatctgtctgctgatgcacaaggagcagcagagaaagccactacagcactggtgcccaaacctggcacactaggaacaaagtccttgttacttcttcgtgggcgttttcaccaagtagacttggaggttaacaaaggacgcaaaggagaggttctaatgggagatagggaattctggccaaagctattccagggctcctagggagcaaaacccaagtctctttctaggagggcattctcccaacgctacctctcccggggatgggaggcggggtcggggggaatggaggctgtcctgctgcccccagctggaaggcttcccagatctggagaagaaagggggagtaactgaatagggaggaggaggaggcctctagggtgcgctcacccagttctggccacttcccacccaccctccacccctgctctgggggcatcgagaggatggggctgttcttatctacagtaaccactgaccctgtaggtcggaattgagtagaggaactaaagataaacttaaatcccaaatctcaaattctttttggggaaaaaatacaaagaatatcatatttattagcagcagaatgccctcctagaaagagacctgtttttttgctgagtaacaatttggatacctctaagaattatcaaaatatcccttcgtgctgtgtattcagattgtttcagcctttctgtgtaaaaagtggcc

SEQ ID NO: 182 - DNA, 2001 bp, Homo sapiens
gaatgggtagaataaaagtttgaaattttccacttcacttctttgcacaatctgaggcagcctctgaaaacacgatgccaagagccctaggtaatagcaggacaccaggaaaatttggttggttaaggcagtatgactccagagttctgctaataacaacctgaaaccaccatagtggcagaggaattacattttttaaaaaaaaaaattctttcaacagaacacaaagactgaaataggaggtcactacaaccgcgggagctgccgccccgccctgcagggagcacctggcctgggacccgcaggcattctctacaaggggtgcagctgtgcaaatgctcacaggtgacagaaacagagcatctcctgcccatcacttcatccaacagccagaggtgacgaagacgaccctcctgagtgaggactgagggtccacaccgcccccccaccccacacaccatagagggaccacagaatccagctcagcccctcttgtcagccctggtaaacgcaggcagtgatgtcacccagaccacaccccttcccccaatgccacttcagggggactcagagtcagagacttggtctgaggggagcagaagcaatctgcagaggatggcggtccaggctcagccaggcatcaacttcaggaccctgagggatgaccgaaggccccgcccacccacccccaactcccccgaccccaccaggatctacagcctcaggacccccgtcccaatccttaccccttgccccatcaccatcttcatgcttacctccacccccatccgatccccatccaggcagaatccagttccacccctgcccggaacccagggtagtaccgttgccaggatgtgacgccactgacttgcgcattggaggtcagaagaccgcgagattctcgccctgagcaacgagcgacggcctgacgtcggcggagggaagccggcccaggctcggtgaggaggcaaggtaagacgctgagggaggactgaggcgggcctcacctcagacagagggcctcaaataatccagtgctgcctctgctgccgggcctgggccaccccgcaggggaagacttccaggctgggtcgccactacctcaccccgccgacccccgccgctttagccacggggaactctggggacagagcttaatgtggccagggcagggctggttagaagaggtcagggcccacgctgtggcaggaatcaaggtcaggaccccgagagggaactgagggcagcctaaccaccaccctcaccaccattcccgtcccccaacaccaaccccacccccatcccccattccccattcccatccccacccccacccctatcctggcagaatccgggctttgcccctggtatcaagtcacggaagctccgggaatggcggccaggcacgtgagtcctgaggttcacatctacggctaagggagggaaggggttcggtatcgcgagtatggccgttgggaggcagcgaaagggcccaggccctcctggaagacagtggagtcctgaggggacccagcatgccaggacagggggcccactgtacccctgtctcaaaccgaggcaccttttcattcggctacgggaatcctagggatgcagacccacttcagcagggggttggggcccagccctgcgaggagtcatggggaggaagaagagggaggactgaggggaccttggagtccagatcagtggcaaccttgggctgggggatgctgggcacagtggccaaatgtgctctgtgctcattgcgccttcagggtgaccagagagttgagggctgtggtctgaagagtgggacttcaggtcagcagagggaggaatcccaggatctgcagggcccaaggtgtacccccaaggggcccctatgtggtggacagatgcagtggtcctaggatctgccaagcatccaggtgaagagactgagggaggattgagggtacccctgggacagaatgcggactgggggccccataaaaa

SEQ ID NO: 183 - DNA, 2001 bp, Homo sapiens
cagcggtccggccgaatctcgccggggtctcctcttcccctgcaccagccagcgcctcctggctggccagtcccaccctggctcacccttcccgaagagcctgccgagaccactcatcgcgagctcgcctctcccgcctccgcccctcagcgtcctccagacttccgccttgctcctgggagggtgatgtctcatcacacagggaccagccttgcccaatccgtcctcagggcgccgccgcgacatcaagaggcgcaaacgagaccaccggtgtcacgtgacccaagccaccagccccagaccgacgcgctcctcttaaagctgccacatcattttctattaagtaaaaagacagttctatctccacctgagtacgtgcttgttctccggaaggaaatgtgcagtgtgatggcacctcagtgtatggggtcagtgggaaaacttcatgctctttacagagctcaggaaagtggggtggtataggttcaagcacttggggttgccctaagtttagatggcaaacctgatgcagtctaatgtctcacattctggtctctgcacctcaggacaaccaaccccatttcacaggctagaaaaatagaaactccccacactgtgggtaaaatctcttctgtcacacacagatgaactttaataaattacaaatgcacctgaaaatgccttcttgatttcctttcagtttaggcctcaaatgggctctcctcaaggctggacctcaaaggcccagtttgggccttcgcaaatgtctctaacccttgacttagagtttgaagattcattccattctggatgtgaatgcaggtaacacctagaaagataagaagtcacatttcattaagctttcagggttctccattattactctgacctttgtgagtgccacctatagggcaaactatccaacctgtatgtatctacctaatccctcctagggaccccaaatggctgttcctcatcactcagtacccagtttgctgacgtctacaatattccgcctctcatcatcaaagaagatcatctgggagaaaggaattccagtcttctgctgcaacctattcaagacagggcaggagtaaccaagctagtatcttggtttcccaggcctctctgatactttctcaccctgctcctcccttatctccgcatacctctcaaagtgtgtgatcttgctgcctggatagatttcccgatgaacaaagtacctgaagaggtcaaagagctccagtagctggttggccccttctatctcacttgtcctgcaaaacggtgtaaaagatgggattagcaaagtgaagggccagggctacgttaacttattaagcaaagttagtaagtataaccagggaaatcaacttgctgtttttccgggatggacaggcatcctcagcctctgtttaactccattgccttcctatttgacacaattggtgctgggccaatgttttagcttttgttttgagacagggtctagctctgttgcccagactggagtgcagcgacccaatcacggctcactgtggtcacgacctcctgggctcaagtgatcctcccacctcagcctcccaagtagctgggactacaggtgcacgccactacaattttttttttttttttggtagagatagggtctccctctattgcctaggctggtctcaaattcctgggctcaagtgatcctcccacctaggcctcccaaaatgctaagattacaggtgtgagtcactgtgtctggccccacttttttttttttttttttttttgagacattgtctcactctgtcacccaggctgaagtgcagtggctccatctcagctcactgcaacctccgcctcccgggttcaagcgattctcctgcctcagcctcccgagtagctaggattacaggtgcgtgccaccacgccctgctaatttttgtatttttagtagagacggggtttcaccatgttgatcaggctggtcttgaactcccgatctcgtga

SEQ ID NO: 184 - DNA, 2001 bp, Homo sapiens
gaccaggacacggaatgtgtgggcttgccaatattttgatatataaatcaactgttttcattatggaagtaacaaaacctcaagggaaatagaaaaaaaaattccctgtaatcatcccacccagctaggaggtctactgtgtgtgtttttgacattttgctgtcctcgttggtgcaagggcagatgtgactgtttatgcctgtggagggcagaggggtttttcttcttcttgtctttttttagagacagggtctccctcttttgcccagcctggatggaatgcagtggcatgatcccagctcactgcagcctcgaacccctggcctcaaaggatcctcctgcctcagcctcccaagtagctgggactacaggcacctgccaccgtgcctggctgatttattttttatttgtttgtttagagatggggtctccctacattggccaggctggtcttgaactcctggcctcaaatgttcctcctgtcttggcctcccaaagttttgggattataggcgtgagccatgttgtcgtcattacagctacattgagatataatttatgtgccacacaatgcatgcctttcaagtgtacagttcagtggcattgaggtatatccacagaaccgtacaaccatcaccacattgagaaaatttttatcaccctaaaaaatcctttacaaaatcctttaccctttagcagtcaccccatgttttgatttttgctttacatgattttacaagcatcttttcctgttgctggaaaatctccttacccatcctctttggtgactgtcagcaaaggctttcgggtggtgaaatagcgtggccaggagtgctctagaacaatggtgttggtgagggctgcagaagctgaggacccacaggcagagagggagggatggtagaagcaggtggtgaggagggactcggggcaggactttgggggtaaaaggaagcaagtctctggaagcagaaccagcagcactgctgatcattgggacgtggagaaaggcgaaggctcagggtgtcagcaaacttcctagggtgggtgagggttgccaggtcaataacgaacagggtcccaggaaaaggcggcagcacaggtttggggagaattagagatgagcttggttttggtcttgttggatttgggggcaccagtctctctcctggcgagagtcggcagtggggtgttaggatctggccctcaggaggctgttagggctgaagcaagagatttgggagttaagggtgtgagggccacgtgggaggcctcgggagcctgtgccgatccagggtgagtgtgaaggaaagaaaaaagagggcccggggaacactggtattttcgggcggtggacgaggatgggacggccacgagcttgtcttaaggtagaaggcagagcggggccgtaggggcagtggcgcgggcgagggtggagaacagcgctgagcctcgctgggaggcggccggagggggctgcagcccccgagggctccttgcagtccggccccgcagcctggagacagctccggtggtgctgcgctgcccgagactgctgcacgagcggggactgcggcctcggctccctgcctgcccggcagccccccatccagtcggcctgcccggggtgcgcagggttagcctggagtgagctcgccccgcccggggttgcgcggccctggggggtcctctcgcggcggctcctgggacctgctggcgccccgggcagagcccggccccatcccaggagtcgcccggactggaaggcagtgcgagcactgggacacagcctccgagctcgagcgtgacgggaagttggggcaatttgttagttatccgccgccaccaagacgcggcacggcgcctggaccggaggggccccgcgcgggcgcgaactttgggctcgggcgagtgggtggtgctccgcccagcccgagacgggcgggcgcgcgggccaatgggtgccgcctcttggccgcggggggccccgacccgtgggtc

SEQ ID NO: 185 - DNA, 2001 bp, Homo sapiens
cattcacaacaacccattaatagatgaagaaattgggctcagagaggttgccatctgtccaaagtcacacaggatgagatggaactggggtttaaaaccaggtctgtttatcctacaaaggatggaaaagaaccaaaccaaggtgcttttcccagtctcatgcagcactcacacttctgacacccaaaggtgtgagagttgtttctccatgcaccaagcaagtttctaacagacaccaagtgagtgtcctctaattcagttccgatgctatcttctggatgatagcatcagattccacaagttaaggcacaagactgccccctcctccccctcaccactgcttcagatgctgaccacaagtcccaggttgtggcctgtgcttctgaccaaccagctgtaaatcaggttcccacaaaacccccctcaggtttgattaatttgatggagtggctcacagaactcaggaaaacacttagtttactggtttgttaaaggatattacaaaggctataaatgtctagccagttggaagagatgcacagggcaaagcatgtgggaaggggtgtggagcttcagtgacctctccaggtataccaccccttaggaagctccatgggttcagctgtccaaatccagttcttttagctttttatagaggcttattgtgccggcctgattgactaaatcattggccatttggaatcaactcagcctttaggcccccacccctccctgcgggtcatgaggtgggtggggacaaaagttgcaaccctctaatcacatgattgatccccctggcaacaagcccccatccagaggctatccaggagcccaccaaaaagtcacctcactggaacaaaagatgctcctctcacccgggaattatcaagggatttaggagttctgtgtcaggaacgagggtcaaagaccaaatatttgaacaaaagattagcacccctatctacgagggttttaggagctctgcgtcagaaactcaggcgtagagaccagatacatatttcttactatatcatcagaccacgtctttaaagcccgttttctttgtacacactctactacactctaattgttagctgtttaagtattagaaaatcattccttagaggcaaaatggtgtagtatggtgacagtgtaagcctgtgctctcttcaccttcctacaactaagtggtgatgtcaagaggaaaggctaggtgtaggtttgagatccgcatttggcagcaggggaagggaagggacagagtgagcccaggaaaaattgtgaagtgggcagagaatcttcactccccttcagactctttccccttctcaggcagctcccccacctctgcactcattttgccccttggtcactgtagagaccccacccaatgcttcggacatgatctagacttatctgttcgcactgcgtttggttatatctgtccccacttgccaaggtccttcacctttgtgggccttgaggtcattgtctctaccagctggtggcctcaggcaagtgactcctctcagggtatttgagcttggagcccctgaattttcaatactaatgcccaaagaacatgagtctaaaatggggttgtctgacttggagataagagcaaagaaagaatcctttggtctcccaatctacagagctgtggatagaaacatttactaatagacagccatgcttcaaagcagtgtttctcatcattttggctggtatatggacaaccctcctccccattgtcaatgccagcagatggaagtgacacagtccatctgttggcccacttcaggggccggctgtgtcttcttactatatcttccttctaaaatacactttctattggagactgctctagtgaaggtgacattctgaggtgaggtggactctctactgtcccttattccaccagccctgaatccaggagacctaggaggggggtgctctgaaggtgtgaattttttccttgttgcaggcacatggtaattgcta

SEQ ID NO: 186 - DNA, 2001 bp, Homo sapiens
cctacgaagtaagcaagctgtgattccaacccagatttgtgggactccaaagcccactactaatccactggaccgtgttcctggaggccacaaatgctctttcctcagctgtcttctcagatcctagaacgggctgatatatggctcaaaagttctttaacggccaggcacggtggctcaagcctgtaatcccagcactttggaaggccaaggcaggaggatcacctgaggtccaggagttcgagaccaacctggccaacatggtgaaaccctgtctctactaaaaatacaaaaattagcaggatgtggtagcaggcgcctgtaatctcagctacttgggaggctgaggcaagagaatcgcttgaacctgggaggcagaggttgcagtgagccgagatcacaccattgcactccagcctgggcgacagagaatccgtctcaaaagaaaaaaaaaagttgtctaacatattaatacaactttcaagccacatgcctcggtctaaaagtaggtgcccccagtacacttttaccccaggtagcaacttccactcaccttaaagtcacatacctggaggaaggggatagggaggaaatctaaacatcttatacttgaatgtaagctccgttatttcacggattcaaatgtggtgaaaggaactatttttttttccttttttttttgagatggagtctcactctgtcgcccaggcgcgatctcggctcactgcaacctcccgtctcccgtgttcaagcaattctcctgcctcagcctccagagtagctaggattacaggcgcccaccaccacgcgcaactgatttttgtatttttagtagagacagggtttcaccatgttggccaggctggtctcaaactcctgacctcaggtgatccgcccgcctcggcctcccagagtgctgggattacaggcgtgagccaccgggaccagacggaactattcttttccacaaaagaacaaacacatacataacttttgccacgtctgaagacgagggttctaatagggcgcgggtggtgtctcaggtattttgaaaggagctacccaagaatctcagcttgtcaggccagtcattccatttgaccctatctggcgcgtattggggccccgacaaggtaaggggttggcatgaagctgcacagtgagataaagctggaccggagctagaacccagaatcaaagccttgccctgtgcggtttctcatcgcctacgcagtcgggccctggcctggcatctccaggcaggtggccttggacggaagcccggcctactggcttcatttgtgggaccccgggggtcctctcctccgcccatggagcctggttactcccacttcctcaagcctagggcctggagaagcctttcctggttcgcggcctcacggtctgcaaggtcctcttacttccctgaagccgctccagccccacttcccgaaggtcccgcttccgggtggctcgcttcgccacctgcggtcccctgcctcggcttgggaacgccaaggacatcccccacggaagcctgggcccacatccccatgcccttgccgcacgtcaccgccccgctggccccgaatacgcctccgccctgcacttcctctctgcagcgccgtctttccggccgggctcccttctaattggccagctcagagcttcctcgggtactcggccactcggggcatcgcggcggcctttctagccgctgtcccaagggttggtctcgcgctttcggctgcgagctctctgtggtgctggcagcgacatgtggcgcctcccgggactcctgggccgaggtaagggacgtccaagcaaccagcgtcactctgggccgcaaagaccgaactggagtgggggcgggcccgaggaaacagccgaagcgatcgtgggccgggttagagccaaggaaggcggggcttgaaagagggcggggaagtcaggggctgctggaatttgagatgtggatgggcctagctagaagagggtgga

SEQ ID NO: 187 - DNA, 2001 bp, Homo sapiens
cgtggcatatcaccaggaatgccactgcttaaactcgtctgaggctgggaagcccacccagaccaacaggcgggccctctggcttccctggagggagctctgtccccagcactcagagaacagagtccccacagcaaaggccctcagggtgcaacggccgtgtcggagggccccttcccatcttctcttcttcagcacatgtgcccagctcctgcacccaatgcccaggggatgcctcccagatgccagcagcatggtgacaggggcaggaggtggcattgtccttgttctggatatccctgggtgaccccggtacgtgactcccctgaactccagcggcctcatctgtgacacagggggccgggccacatcatcctgggcagtgctgctctctgcaattattgagtctggagtctcttcttgtcagagattctctttagggagtttattttccatagactaaagactaaacaatagagaacagagagaggcttctagatacctactgcaaccttagcttctcagaaaatacaggtgcttgggactcttccacaaaatcaggaatttaaatggaaaacgtgggccccaagacagctcctttagtcaacgggtctgagtacacattaaaggagggtggagggagaggggacatagagagggaggaaaggggaggagggcaccaggaagggcggagccaagggagtcctgggggtggatacgcacctgcctgtaaggggccctgtggtcctggcacgtgcctataagaggccctgtcatcgtgtgttaggttgtgacacagctctgccctccccaggtgccttttctgaaccaggaacctaagaggagctctcccaccagggccagggaaggagggcaccaggacgggcagagccaagggggttctggaagtccccggccctggtgggagagctcctctcttaggttcctggttcagaaaaggcacctggggagggcagagctgtgtcacaacctaacacacgatgacagggccccttacaggcacgtcgtatccacccccaggagcacacagacagactctcgtccacatgccctgtctggggacaggagcccagcacccaatgcttgttcctgcttccacccctttgctgacctccccactcatcagggctgcgtgccaggtgaccagggttctaaccctggcttcctccctgacccctcctcctggaaacatccttcctgacccagcctcagtttacctcattgaagggtgaattttaaggacccattcagcttttatctcctgtcactccatagacatcaagacttaacatcccatccctcccactcccagctgtcattccccccccccaatcttcggcccactccctgtccaggggcccaggtccccagcgcgggcaacccctctgtccccattcccgggacctggagcccgcgagggctcaggccggccaggatggagttctctatcccagttcaatttcccccgcctttttgatgtcacatcctgtcccagcctgcttccccgccggccgccgccctcctccccgggagagagcgaggcgcgcgggtccctctgcgccacccccgcccccgccccttccgagcaaacttttggcacccaccgcagcccagcgcgcgttcgtgctccgcagggcgcgcctctctccgccaatgccaggcgcgcgggggagccattaggaggcgaggagagaggagggcgcagctcccgcccagcccagccctgcccagccctgcccggaggcagacgcgccggaaccgggacgcgataaatatgcagagcggaggcttcgcgcagcagagcccgcgcgccgcccgctccgggtgctgaatccaggcgtggggacacgagccaggcgccgccgccggagccagcggagccggggccagagccggagcgcgtccgcgtccacgcagccgccggccggccagcacccagggccctgcatgccaggtcgttggaggtggcagcgagacatg

SEQ ID NO: 188 - DNA, 2001 bp, Homo sapiens
ctccggcagcttgtcgccccacgcttgggaggccagtccgcacgctcggtcgcgggccgtgcgccctcagatgaagcccgtgcgggccggccccagcggcgccccacctccgggccgcctcccaggcccgcctcggccctcctgcctctcagcgatcgccacctcgcgacccagcctgtggacgtgccgcggcgcccgcgccggcagcccggggcctcggaccggttccttcccgccgcgtgaggctgacgcaccgaccagcttcccggctcggccccgcccggtccgctgcgcccgcgcccgtccccgcccccggctccctgacgcccccggcctggagacgcaacccgacgcgcacaaccccagaccctgcgcgggagtcgggggtccttccagttcccagagcgagagcgaaagccggaggggcctgtgttttcctgcggggttggggggcggggggtgcccgccccgcgccgccccgcccagccccagcccgctcactcccggccaatccttccgtatctcaatgcgagacaatcaaatcgtacgaattacgtgtccaccgtgttcccggcggagtcttaaagctgaaagggatattagagagcatctagaccagggcagcatatgtcatttatatttttagtagccacagcaaaagagtaaaaataacaggtacattttatttttaaaatatttttatttaaccccgaatgtgcaaaatattatttcaaaatataaccaacattaaatttattcataagatatttgactttttttgtctttgaaatccagtgtgtattcgatgcgaactagacccatttcatagtcaaatgtggctggctagtgcccactgtattagacagcgagggtggaagccacccgctcatatcataggaagaaacaggcctagagaagtgaggtaaggtgagcaaggattggaggtcctctgatacccagtcctggtcttttaacattcagagttgggactagtctatccgctttttttttttccagacagggtctccctgggtcacccaggctggcctgcaatgatgcgatcgcagctcactgcaacctcgaactcctgggctcaagcgatcctcccacctaagcctcctgagtagcatgaactacaggcgtgcgccaccacgcctggctaattttatttttagtagaaacgaggtttcaccatgttgcccaggctggtctcaaactcctcaagagcgattctcccgcctcagcctcccaaagtgctgggattactttttttttttttttcgaggcggagtctcgctctgtcgcccaggctggagtgcagtggcgtcatcttggcttactgcaatctctgcctcccaggttcaagagattctcctgcctgagcttccccagtagctgggattacaggcgtaagccaccacacactcggctaatttttgtattttcagtagagaaggggttttgccatgttggccaggctggtctctagctctgagctcaggtgacccacccatctcagcctcccaaagtgctagaattgcagacatgagccaccgcacctggccttactttttgttttttgtttgtttgtttgtttgttgttttaagaagacttttttttgagacactctcacttcctcacccaggctggagtgcagtggcgcaatctcgggtcactgcaacctccgcttcccgggtttaagtgattcttacgcctcagctacccgagtagctgggattacaggcctgcgccaccatacccagctaacttttgtatttttagtagacatgtggtttcaccatgttggccaggctggtctcgaactcctgaccttaagtgatctgcctgcctctgcctcccaaagtgctgggattacaggtgtgagccacttcaccaagcctaagaaggtagtttctaggtgaaactggcagctttccgacatcttgcatgcacattaaaagttgaaatagttcaaatgacttaaactcatgtattttact

SEQ ID NO: 189 - DNA, 2001 bp, Homo sapiens
cgtccgcgggcgtcaatagcgactttcagcacaaaacaaagatggcggcggcggcatctcggaaatgcccggatgagactgctaacccctccgacgcgctcggccccgcccccttgggaacggtctctcgggttgataagggacgcacgcccgaagaactgcctttaaccagcactggggacagcggcacgcaggtggccggggttggcgaacagccactgcagccgcgcctgcggccgggaagcgagcattttcgcgccctgcacacccagcagccgcaggaggcggcccgaggcgcgccgcgggggagccgggggcggggcgacagtgggaggcgacgcagccgagcgccggcgcccgggggtggagcctccgcccgctcccaggctcccaggctgcggggcccgccctcgggcgccaccgcctccgcccgcgccgcagcaaaatgagccgggccgcgcgccccagaccgccgcgcgctcggccgcccccactgccgagaggcccgcagtcgggccggtggcgcacgagccgggcgggcgaccgcgggaggcgtccgcgtcatctcgcgtcacctcgccccgcccgcttcccaggcttcgagtcccgccctgcgcgcaggcgcaccggcgctgctcggatcctcccttttcggagatttgaatttcccccagcgaggcgagtgaggcgaaatacccgtatggtgatagctggccttttcgcgccaatactgtaagtgctcaccgcagccctgggaggtgggtcccggcctcgcccccacgtttcgcctcctctaggaggccgcccgggcgcggcggcggcggcggcggcggcggcatccgccctgccacgcgcaggccgcagcgcggggacccaagctgctgcccgggagcggcagtcgccagtagaaatggacagaccggtcctgtcagagcctgtggcttttgttgtaaacgtctagaagttcagttaaattacaagatctaccagatctaccggcgggcagaattattactttgaagttactgaagttatgcgtcctgctggaagaccttgtaatttaatgcgttagtaaagaagcacacgtcaccctctcacacattttcaacaatatttttaaagtcgcctctcaacatattacatgtattttgtaatcgtctgtgttttagttcatgcagttggaagtatccatttgagaagggacaaagagaagaattcttgcctgtctgtgccaccacctgctctttgtgaccttggctagttacctcacatatgtccccaagtcccccacccatggtaaataagaaaactaaggcctgatgcgttggctcatgcctgtaatcccaacactttggaaggctgaggtgggaggattgcttgagcctagccggggcaacatagcgagacccggaggcatggtggcacacgcctgtggtcccaggtacttggaacgctgaggtgggaggatggcttgagccagggagttcagggctgcagtgagctgagatcgcgccactgctccagcctgggcgacagagcaagaccctgtctcaaaaacacacacacacacacacacacacacaaaagcagctgctggtttccaagggccttgcctggaatcagtccagaagctaaattaatgttaattgatattcctttatccacagaaagacagcaagaaaggtcatgcggagcgggggacataactaaccataaatgctaaaggcctggaggtgacagtgcctgctgtttaaacgcgtgcgggagagtctggggcctttagccaggagattgtcaggcttcccagggcttccctgaaagaccccagacccaggggccagtgctgttgaggtgttggctttcccatgccaggcgctgggggaagctgtgacgagatttgagtgtcacataataaggatcatggcaattcaaaggtacagggaagctaggctataaggtaaccagctcggttttcctgggggtcttggacaggtgctttga

SEQ ID NO: 190 - DNA, 2001 bp, Homo sapiens
tgggattgcaggcgtgagccaccacgcccagccctattattatttttgacaacaacagcaaagatctgcccatccgactgcaggaatttaatacacagcctttgtcagtggtagaaattctatcactgtatgactcatgagtacataacaatgtgctataccatacttcctctttgagggataaagcccctgcactctaagggaggatttcattatgcatttctcttatatcaccagaaaattatttagtggaagacagtttaatatttgacagctttgaaccatttttttttctctttcaaccagtctttttacattgtctgcttaaaaacaggaaaccacatatctccaccccagcacaacccaccatccaccagcacagaccgtatcattttggatctacagtctttttcttccaagcctggccaggtgcctgctcctactttgcaggaaacccctccaggcactcccggtgcgtgttctcagagcccttctgagaatattcaggattgcacttcctgggctgcagcgcaccctgtaccctccttttttaagcctcctcccctgagtcactgctacccttccctcctctaacttccccttctctgcagcctccttagcaatttcttatcataaagtttgttttaaagaagaactgcccctctaaaaaaataataaactgtcgagggcggtagccccgtctagaggtttccgactttgctctgtgcccacctgaagcctccccagcccgaggtatttgaacttacattcagtgcttcacgtctctgtccggggtgcctctgtctctcaactctgacttctgagtactggcagttagaacaggaaggaagtcaatgagaaaaaaaaatgttttagacctacttgaagaggaaaccctgggctgtctcaaatcctgttcctttgcactgcctatctcttgatcagccaacttgagcagcattcttgtcaattaatggcacatgtgcgagcatgacagcccgtgtgacgtggagatgcatgaatgtacacgcaagagtgtggagcctatagctatgggtattatgaataattgtgagggtatttctgataatcaggtcattgcagtgcataagcctatggaggtaaggagcctgaaaccagtagcaataaattcacatcggctttactttgtaccactcacttctgttcccactgggtccccgccagcttcattatcaaccagatacgttgaaagtttgctggcagtgaatgtagagtgctaagaaatgatgattgttaactgcatatactacggtcctaaatttaaaaatgtgtccttaaacagatttttggagttttgttggttataatttcaagacgtagctttgtattgcactacaaacagtcaaatattgtttttaatagttttggctgatttttcttcaaatgaaacaaacaagcaagcgaaagtactgtctttgatgaaacctaggaaagaaattacaaaaggaagtaaaacttcattaattcatagtcttccaactcagaatttgtgacaacttaacaagtgccaaaatagactttgtaccatatttgaggaaatggtttatgaagtaatttaaatgggaacaagtctcggaggccatgtttaaaccatctgaggaacagtttttaagcagccaagtcacattcatgctgtatattcccgtagctgttaccaataataaatgcttatttaggttaatatttcaatattcagtatttacactacatcaatatatttagatctgactaagcacacaaatttggagaaactgacgtttattttgttttcacctaaatgtactgcatcttcttcatttccctttgtaaattgataccacagggtttgccttctacttagagtcaatgaaatcggaataatcaagggagtcatttcacgtgagaagagattagcctagaaaaatatgaaaaggaaaatagtatataattaaaatgagcaaaaatactaaaagcctaata

SEQ ID NO: 191 - DNA, 2001 bp, Homo sapiens
agccaaaagattggacacccctgattgagaacaatgcctggaactttgtagctctcaaaagtttgttcaatgaatgaatgagtgaatgcttccaatacaaaaccttagaacaggcaagtcaaaatgtcaccaaaaggtgggaagaagaggcattcacggtactggttgagtgcatagtaaataaggtacttgcacaactatgggcaacaaagagacggcttgggaattatcaaacaccaattttgattttgatgagaacagtcataacccaatatttgaagattttgatggttgcttcttctttgcccctctaaccaccccccccaccccagaacgatgaagggttaaccaaaacaccaccaaaggcaactacttattacctttgttattcccacataaaaaattgagcagacctattgtttaagcagaagaggcagtccctcaatgaagcaatatcctgtttgaataatttaggatggggcataggccagcccacacagcgcatttgtgtgacagggcgtcccactcagacgtctccatttccggtatctagagccggctgccctctaaaaatccccctgttacagtcgggatagaaaacaaaaagcttaatttaccaggaggctaaacagattgtgccttcttaggcaaatgttccacctgcttggccaattagcttcctctcccgaaagactcgttcaacaggtttcctttttctcttctctgcctcctgggttggggaaagaggctgtcacttcgcttcgaccctccccttccctccctctcggcttttccacagtgggtcggaccaatcgcgcccgctccgacgtgtccaggtccgcggccccgggagcttggcgcggcccggcctggatgcgctgggcggagggtgcaggggagggcacggcgccgcttgcttggcctgcgcacccggacctagaagccgggacgagccggggcagagccaggcgcgcggaagtctgaaggtgccctccagacacggccccgatgactgaactacagcaagatgtggaagacacaaagcctgcgaaagtgctcgggaagagggagagcaaacttggctcagcccagtgagtattctcagctgggacccatccaaggaggtgggaggagcgcagcctaaacttctctgccccgggacgcattttgggtggatgcggatttctggagctcctatatggacaatcttgtggcgcgctctctctgtctctcactctctctctctgtctagatattcagttaaggagatggtttagattgattgaaaagtctactgtaggatttaaacaaattctcttaattcaaaactcactggatgttgggaaaataattttgtagaaaataagcctaaccacattgaagctcaaagtcccaacacagcccttcgtttagaatgaccattttcacaaattcattttagttcagcaaataattgagtatctacttgttcaataacgatgggcaatacaaggataaatgttattttggatctgcccttctggaacttcaagagcagagtggggatgggggaggcacaaatgaatattactgtacttcaagggactttggtgaccattgccacattaaacacacagacaaggtatgataaagagtttagcctataattgagtcttctctacttttaaaatatttatctattttgcagtctttggtaaaattggtttgggaggagaggttgtttttttcctacaaagtggtattttctaagcctactgaagtcacttctaattcagtctatgattttagacactaacccttctgagctctaaaactcttgttctggtatcaggatatgagtaatttaggaaggagggatatgtattatcataaggttatactgatgtgataattccactttgaaatcattttaagttgccaaatttttgtgcttagaaacccggtctaacttctgttttgaacttcataacttacattaattatcactaggctcaaagtctt

SEQ ID NO: 192 - DNA, 2001 bp, Homo sapiens
aattctctcaacagtattttgaggtaatggcactggccgatccagtatcccggctttttttcttaagagccaggtagcaccaacattttcatgctaaataaacattaggaaagctaaaattcttgttgacttatacctgccatgcaaggtggaataaatatgcatcaagagtccaaatgaaacagaaaactggggagagaagaagattaagagaggcactttgcaatttattaaactgttaatttgccttacatgcctttttaaaagaaagagctttggctcaaatgaagaaagaggaatataagaatgaagattaaatgggaaggaggcaaaaacttgacaggtgaaaatgacttggaaatgatagatacataaaaacaaacaaacaaaaacctacaaacatgatcatagctaagagggaggaaaatgaaaggctatgtactaattcacatatattgacaaaagaaaaaagggtgagtccataaacgctctgacccttgttgtagatgtgaagcgttccacttccagaacttgtgcttttattggacagccggagagatacgtctgtatattaggctccttaaatccttgagtgttataaatagcagagaaagggatatacactaaaaaaaaaagtaaataaagttaatgcaatatatgattagcataacttgtcttttacaggcattcttttaagaatcacgtgttaaaacaagtaagcctcttataaaatgatctgagcccggtgttacatctgcaaggaaaatcaatcatttgctggttacttcaaatttgtggtcttccgaccaaccgagataaactcttttcttccccgctcagatcatccgtctttaccttcttgtatcttgggatcgctgggagacacgtcgtagtctacctcctctccctcaaagtggagttcccgcgtgtccagattttctatgatattactcatgtcagcagcaattttctgcagtgcagaggtatttacccgtggctcgtttttcagtgacatgttaactttggacagagtaaaagcaaaggggctaggaaagaagaaaacggttacaaagacttagcattttgataacataaaatgctattccacaatttgaatccagacttagatacagcatatggaaacaattcagcctgctctgtgtgagcctcagttatccaaaacaaccttttagtttacgttcctgtttaacatttaaaggattcctaacttgtaaattaaaacagagtgcttaattgaaaacaagagaaaaaaaaaagaaaaaaaaaagcaacaccacattgaatgccaacatttctctcatccaactttattaacttggataagtaattcaagggagattggtttgggaaagcttattcagagaaagaaatagattatttgctgtaataaatacagctgaagaaagaatatcatgaaaaataagcaactctaaatatttcctaaagaaaatttagagtatttcacatattgaaatatatagatttagaaatatttttatgatagcataatatttctacattttctctgtaggtgatatcccaagcaaagtgtatattacctcttcaagaaaggagattttggccatgactttacgtgaggtataatgacgtatgtgtcttgaaaggaaattgattaatatacctattccgaaagacatctgaggtgctagaattatacttccctcctttctttttctttaaagaaaaccagttctatttcattctgacatttaataacacaagtcgttgcacatcagtacccagggcagaagagttcacatggatattatttttcatgtaaaatattgagattcaagctttgcacactgtcatcagagttggatgaaacatctgttttgcctgatgtagaaataatctgttaaaaccaggccctagaatggaaggacgctcataccagtaactctttggcacatgttcctttcttactgcaaatcacaaacactgagcatccaccacatg

SEQ ID NO: 193 - DNA, 2001 bp, Homo sapiens
acttttatgtttttatatggttaaagtaaattcaaagaaggataatatttcatgacatgtgaaaatgatatgaaactcaaatttcaatgtccgtgaataaagtgacgactgtttttgtcattttatatatatatattttttttgcagagggactggcttgcactttttaaacattagttatattattctcagtacaaccctataagataaaattcattcccattttactgatgagcaaactgcagcttatgtttcagccaaagtcagacagttcattaagggcagagtcatccatggcaatctgcctgtccagatattacactttcccccggggtaaaagcactggctctaagaatagtgagaacaaaacatacttataaaagggagttgggggctgggaatggtggctcacgcctgtaatcccagcactttgggaggccgaggcgggtggatcacttgagatcaggagttcgagatcagcctggccaacatggtgaaaccctgtctctactaaaagtacaaaaattagccaggcgtggtggcgggtgcctgtaatcccagttattcgggaggctaagacaggagaatcacttgagtccaggaagcggaggttgcagtgagtggagatcgcaccattgcactccaggctgggcgacagagcgatactctgtctcaaaaaaaaaaagagagttgggaagtgttttgaatgcttagagggcagcttgggaactcaattagaagaagggacggtttaagcagacatatttctaagagacttatatgagttaactcatttgatcctcagcactcctccatgagttggatgctattattaacaacccaatattagataaaggatgaccttcttaaagacctcttcccaggaaaaagactgtgtgtgggtactctgaagctttggctcttctggtggggacaccactgggtttccaaaggctcatccagccaagcgaagtatctatgacctcgtcgtgttcagcgcgcttagggaaggattctatggactgcacttttaccctggtgtcctgcattcttgctccaagacttctcttaccactctcattcccctaaatgcagcttctgacaccttctagctacgagttcttgggctcctccttcgtgacttccttttaagataatttctgagttacaacaaatcagcatttgaagaaatgcagcagataagttgaaaattcaagtgctgaggaatgtaaaggcccaaaacaatcccgtagaaagcacagaggcagatgacagtcttccgttaaagatttcactgctgtagatttctgagagtgctgtgtagctgtcctctttatcctggtgtttgccacttcgaacagtcactttcccacttggaacttacttgtggctttgctgacaaaacaggaagtgatttgaatacagtttgtttttatgacctttcaacctcaggaatttgaaaagttttagcagtggatttacagccttgcttaatacttctatttcttacaaaacaaccgtataggggttaaagtatcagtgcaaagtcccgttcaaatattgactcagtaatgagctctacaagccagcctttgtggcatgttaaattcaaaacaagcttagcatttttttttttttttttggatagaaactggtgggggtggggaaagaaagagggaactgggaagagaaggaggggaacttaaaccttgcttcctgctctgtctttctcagaaaaccaaatatggcatcttccatgaggagcttgttttctgaccacgggaaatatgttgaatctttccggaggtttctcaaccattccacggaacaccagtgcatgcaggaattcatggacaagaagctgccaggcataataggaaggtaacaaaagggacgttgttgtcaaagggacaagcctcagactggctgtgcgtcagtgatagatgggtctcgctcagcctcgcaggctgggcacagggataagggcgaattactgatagc

SEQ ID NO: 194 - DNA, 2001 bp, Homo sapiens
ggcaatgaattcagggccacctgatggaaaacttgccttcctctgtccccaacacaacgaccagtacctccctgacccacccgctttgagaacctcggcttcacgcagtctcacggcatgttcttcccatccaagcgcaccacaaacgcattcccggctactccctccttccccggggactccggcgcctccataactcctcccacctccagtcccagctcctggaggatgtcagagacccccagagtctcaggagttcctcagctccagggccctttgttccttctctcatcagccacatgtttccacaggtcccctcagccttggtcatcaccgggcactgtcccaccacccagaacaacgttcagaaactcccctctctctgaacccaacctccctcctcaacttccacggcgttcactccttttttctctcaccgcatgtcctccgaccacacgcaggcctcagtcacctcctcctgcctcggtcacccccctcctgcctcagtcaacccctctcctgcctcggtcacccccatcctgcctcagtcagcccccctcctgcctcggtcacccccctcctgcctcggtcacccccctcctgccttggtcacccccctcctgcctgtgctctgcctcagttaccccctctcctgcccatgttctgcctgttaccccaactcctgcccttggtcaccctgctcctgcctgtgctcggcctcagtcacccccctcctgcctcagtcagcccctctcctacctatgctctgccttggtcaccccctctcctgcctcagccaccccctctcttgatttcatcctcacccagcttggatgtcactgtccctcatttcagctgatttcctcctccctaccccccactgtcatgccagcccagcagaactccagccaggcgtgacttcaatcatccacctgctccaggggacattcgggcagaccaccctgtgcaaaggctgtgacgttgccactctggaataacacggtcaccaacgtgtagagaaccctcagcctgcagggcgatcccacaggtcagcatggcttcccactcgtcaaacctcctctcctctctttaaacttcacacctcctcccctgtcccctctgtggtaactgcttcctaacgagcccttgacgatcccagcctcccggtgctcacaccctgtgtaggcccgacgcacacacggaattagagctgacctgtgtgaccagcagaatatggctgaggagacagcgggggtgggtgcaggggtgactttcaggaccaggtcttggaaggcactacagctgcttccgccttggtctcctggatgttgctctagaggacaccagttgccatggtgggaagacgctcaagtggccctgtggagaggcctatgtggagaggaactgaggcctcccaccgacggccagctctgactcgcaggccatggagtggcccccggtacccgttgagccttcagatgatgcagacccagctgacagccttctgcaagtttccagccagagccttccagccaagctgctcctgaattcctgaaacacaaaaacagtgagagacgatacatggtcattgtttcaagtgactaaggtttgagatcatttgttaggcagcaagagatgacaaatcatctcattttcagcagacaatcccgatcctgcttctcagggtaaagatcatgaggaggaatctactttcctgtgttgctgccagcactgcaacctgtgccccacccttctgcctcctcccatcgcagacaaggagctgcctgccttctccgcagggcttgctgggcagttctggcctggggtccaccctcctgcttttgaggagctgtctgtgctcgaagctctctcctccgccctttcccttctagctctcccgtgctgctagacccttcctcatgcccctcaagcctgatgtggcatcttctgtagtgcgatgagcccatccttggggtacagctttctttccggttactgccc

SEQ ID NO: 195 - DNA, 2001 bp, Homo sapiens
gtgtgtgtgtgtgtgttttaaggctccccgggtaaccgtgatgctgaccagctttaggaaccactgtcaatgaaagcagacatagtacaaggtctaggaagccccaggttacgtgtatataggaagaaatatatcaggtgaagccttaccagtcccctgccaggggcctgcctttgctctggcgttgggagtctgcacacagaatgtggaacttggaacgcacgctggaacctggcactgttgcttccttctttgtacagatgacccttgcagtctgaccaggcagcttgaatccaaagagccaggctgtcactggggaaggctggccaagtctgccaagaggaggctaaaagggatttccttggcacccggggccatgacgggggttatccttgcacactcagaatctcagttaagttggcacatggtagcctttagctctggggcgggctcaccttaacaggtatgcatggtgacccaggcagccagtttgctaatttactaatgaaaaatacagctgtcatgttggcggatcatggtctcctttccctctatgtgagttgcacgtgaagccaaatctgaaggttgtttaataagaaaccagcacaagcaaaattcaccagatggtctttaagagagccacacccacaacaggggatgctgtgttccagctcatgagggagggcatcctcttcatgcccagacagggcctaatgggcatcaccagcttccacggtgtcggcgtggactctgcagtcagactgacggaattcagatcctggttctgcacttaccatttgtacgactttggcaaagcagtatacccctcaagccctcagtttcttcatctgtataataaaaataatagcagctacatcattggattattgtaaggattaaatgaaagaagtgagacgtttaacacaatgttaggcatggttaatcttcatgtctggtagttattattatcatcatgaccactactactattattgtcctcgtctcctctgatttaggcctcactcttcatcatagttgttctgtgtgtacaggatacctatgaatgattaggtttccactcactaaagtggaaacatattctagtcttgcctgtttgtaataaacttagcagccctgcctggagttggtgaccctcctctccacaacacttcatggtacctttgcccacagtctcaggtgaccagtcatatcctttgtgatggtggtggaagctcctgaatcccaggattctgaactggggatgaggtggaagtcatctcacagcctgtcacacatccttgtgcctgaatctgcatcctgagttcttgtgtcccctccctgcactcccacttctaaatagctcattagtgctgggacatgaaggcagctgcagggcagggcagggttaaaaggcagggattgagcaagtccctctgcaggaaagcgtgtgcccatgtaatgtgtctagcagatgtaacttctcgtctgtctgggatggagattatgagcccacatgggcctgtgacttgagcaaaacaacaaaacaaaccaagaaatgtgaaacgcacatgcctgtccttatacgagggctcagacacttctaccctgctgaagcaggtggatgctctaagtggcacccactccgcctgtgctggggacagcagaaggggccctccaagccagactcggctctcgtgaacccttcacgactggggcctccccgctaaaacagcctcattcccacttaacagctttggctcatgctgttgtcttggctataatgcttccctccacttaataaatcctgcccatatattcaggcccagtttaagccccaccactccttgatctctggactcctcctccaaatgacagcatggtctgttttccagatacaattcagagctggattcggtgtttgtttttccagttatttttgcttcctctaggaaacttctttgaaaatgagactctccttccctagcaggggctgggtgcaga

SEQ ID NO: 196 - DNA, 2001 bp, Homo sapiens
acagatttctctttcccttgctcaaatcagcattgcattttcttcagcaatgaagtatcatagtatcagaagaaggaggggaggtcctaaaggaaaactgaggcattaaagggaatcttctttggagacagaaagtgttcctttgatccttatccttcctgaattaccatttcatctgtactggttttctgtctaagttccattgctaaccaggttaagtaaaggaattggtcacctcctcgggcctcagtttccacatctgtaaagtgaagggtctagactgtggtctctatattcactttctgtctgtagattgctcatatttctgcattccttctattggctgtccatttccatagcacttttctgcattgcttcttgtacttttcacctcttatttctgaattgtggtcccactattttactgaaattaacctctcagacatcactgcacatctctaatcatcatttcttttattttttcccctataccctcttggttacattcaatactgacaacctcagtctgatgagtctgatcgagtttcagacagttgctttctaagaaattttctacatctcatgttgctccccttttctcttccacttatagctcctgtgccctacctttgtcatttcttatagttttgtgcttggtatttacgactctctctcttttttttttttttttgacagggtctcgttttgtcacccaggccagagtgcagtgacatgatcacagctcactgcagcctcaacctcccaggctcaagtgatcctcccacctcagcctaccaaatatctgggactacaggcacacgccactatgcctggctaatctgtgtatttttttgtagagttggggtttgccatgttgtccaggctggtcttgaatgcctgggctcgacagatcctcccaccttggcctcccaaagtgctgggattataggcaggagccacctcactgtcttaatgtaaattttcccgcactgacttgtccgttctgttttactacttccctctttcccatctctttgcctttgttcatgttattctttccacctggttttcttcttcagactaaaaattataacaaatcatttgagatcttgcatttgccaagaatgattgacatcactttcaattacctagattgaaaatgaagttggcaaaaatataaattttcatgacttaataaatgagcttgcggaaaaaaagccagaaagatttgtcaatcaatttaatacattattaaagtattatggtttattgttttacataaaattaggacaccaaaacattatttctttgctgtttgtagtttatgctgttatatatgtgtacccgttacctcattacatgttataaataataagactttttagaagaaaaagatttatattttactatttctttttttttttttttgagatggagtctcgctctgtcgcccaggctggagtgcagtggtgccatctcggctcactgcaacctctgcctcccgagttcaagtgattctcctgcctcagcctcccaagtaggtgggattacaggtgcctgccaccatgcccggctaatttttgtatttttagtaaagacggggtttcaccatgttggccaggctggtctcaaactcctgacctcagaccacccaccttggtctcccaaagtgttgggattatagacttgagccaccgcgctggacctattttaatatttctaactgaactttttttctgacttttcaacaggagacctcacattttcattttgcactgggccttgtaaattatgtagccagcccgaattgttgggtctctctgaggaagctgaagaatctcagagaaaatacctccagatagaattgtttgagtgttctccacaaaaagggagctcactgtacaaccactctggaaggaaacacagacacttcacacaaagctttcaaatgtttaaggtcttactcaaaacgaacataatttaaaatatcacgatacattg

SEQ ID NO: 197 - DNA, 2001 bp, Homo sapiens
aagcatttcagactttagttttggtagtacaactcttgtaaccattcaagtgctgctttgctgtcatttcaactcttaaaagaagggaatgggggaggggcaagagagtcttctaagtaaacaaagatcccaaatccatgaaacctaggatttatcatggtatgtacattatgaaattccatatattgtaaactgtgtagcatgtcaagatttcacattagcaggtcctagtgtactcttcaatgcacatacggtttttatatgcatattaataaaggcccatctttgggagaagaaaaaaggctccctttcaaatgaaatcaagcccaggtaaagctgaaacccaacccacagagtactggcaccgttttcaggccaaaagaagacaagggtttcaatttcatcatcttgtaatgctgataaatgcgatgctgtgactggcaaaatttgcagattacataggtctttctaaacgtgtgtaaatatgcaaatatgtattccaatttatgtgcaattccaaacaatttgctgtttgaaattaaagaccaacaactcttctgtgagacaataattcgggtcatttacatgaaaagtcttaaacagcaaaaatgctacttgcaatttacatcatacagtgctcccatcacagagaacaatggcttcctcaatggaagttaacgcagtagagaggtatactgagattttttttttaacttttcagcacagtttgcaaatactaaccaatgaatcaaatttaaaataaaatagaaatgtttcttatgcttggtcatcactgctacttgagtgttatttttgtgtcttcagattcctatcaaacaggagtgaacctttttaacaggttatggtgacattttttcagctccttccattttgtcctatttccagtgtcagcacaattagcttgacttgggggtttctgtctggttttaaaaacctttcaatggagtgcacgtctgtgaacaataaaggaacgaggacacggggctgggaaaacagcatacctcatctggaatggaaatctatcgtctctcgcccttgcacgggtcacgattatacaccttcgtgacctttagcgggcttacatatgtatctttttgtgttccgcaggctatgaccacctgctgtatgtacaatatgtgcttgttacatttgtacaccatctcccttctagaatttaaagacctagctattacatcttgtttactcaatatcattcccccattaaggaaaaaaaaaaacctacatgaactgaacatgccatgtttcaatctggccccagtggctttttctctgaaagcaaacgtgtgtcttttacaccagggctttctccccaccccagggggtgtcttccatccttttgtggctcagttgaaggcgaaaagggctccaaaccactaactaaccagaggagagccccttcttccacctccagggagaatttcagatttaatttgtccgaagatagcgtgctctcttcttactcatttgccatcattacgaggaaaacaaaccaccaccttggcttcaagatcctgggtagaggctcacggtcttttcaaccatctttggcgaggccttgcttccttccactcgagtaagtcgcggcttggggccacggaaaggactgggtgagagcccgcaatgtcgttggaaaaaagcagccctccctgcgccccctcaccagccccaccaccccgctccactcccggggcgaagccgcgagccagcggccccgtatgggcgagacttggcaggtccgcggggcgccttctcgccgccgccggactcggggctccgcagcggccgggcgtcggcgcccgccggccgcggggcttcgcgctgccgccgcccggtcccggagctcccttcccccaccggccgccccctccccctcgcccgcgctcccttttgttcgctcgccgcccgggccgagccgccgccgccgccgccgcccggcccggcggcgaggccgctccaccg

SEQ ID NO: 198 - DNA, 2001 bp, Homo sapiens
tgtatgtcccattattgctggccccatactagacatgtgtctgcactcaattgatagctttataatggatgaatgattaagcctttttatttaaagctagttatgttcccacctgagaggctattcgcaggggaaaaacatttgattcaatcctctttaagaaagcagtttggccgggtgcaatggctcacatctggaatcccagcactttgggaggccgaggcaggcagatcacctgaggtcaggagtttgagaccagcctggccaacatggtgaaaccccgtctctactaaaaatacaaaaattagctgggcatggtggcgtgcccctgtatgccagctactcaggaggctgaggcaagagaatcacttgaacccaggaggtggaggttgcagagagccaagattgtgccactgcactccagcctgggtgacagagcaagactctgtctcaaagcaaaaaaaaaaaaaagagagaagaaaaaaaaaagtagtttgatcagagagttgatctatgttgttcatattatgaaaagactttaaatcgttttgtgagaatacaatgttgatcaacttatatgaattcagattcaagaagagactgcagaggatcagggttattaaatagcttcctggcagtcaaagctgttctggatgcaatgggctgccttagaaagtagcttctgtccctagtattcaagctaagaggacaatcacttgcttggattatctgcaacatctctgccatcagatggattctcagactggataatctttaagtttcttctaataatcaattttatgagtctgtgctgtgaagagtggtgtctcaagaccaagggaacaatagaggaagacgtattcagctttggtcttgtgccaacatttcctagttttaggttagcaaattcagaaatctgttaaatgtttattgagtagcttctatggtccatacactgtcttaagtgctggggaattaacagtgaccaagaattttagatcgtgacttcatggagctcataatgctaacaattgacattggacaagtaactacaagtttgatgaatcttaccaaaggaataattgaaggagcctataataaggcgatttggcaaagttggagtcaggagggttggggaaaagctatcaaggagagatatttaagatgagaccttagttcttcaaagattggtagaatctgcttagtgaagaagataatgaagtgagtagttcttaaatttcaatttatgagtataaaacctgagggattagaattttttcttaaaggtgaaccagatttcagtctgagtataaaacgtttttatgtaataaatctgtttaacaaataaaattagatccagcaatcccactgttgggtatacatccaaaggaaatgaaatcagtatgttaatgaggtatctgcactccacgcttattgcagcactattcactagagccaacatatggaatcaacctaagtgtctatcaatggatgaatgtgtaaagaaattgtagatatatacacaatggaatactatccatccttaaacaaagaaggaggctgggcaagggagctcatgcctatgatcctagcgctttgggaggccaaggcaggaagatcccttgaggccaggaattttagaccagcctaagcaacatagaaagaccccatgtctacaaaaaaaatttttaaacatgatcagggcatggtgatgcatgcctgtaatccttgctatatgtgaggctgaggtgggaggattgttgagcccgggagtttgagggtgcagtgagctatgatcatgccactgcactccagcagagaccacccagaatagagatgcatctactcccagccaggggtaagagagagggaaccagcaaaacttaaccagcaaaactgcccttaacagaaaatagtgcatcctagccatcctccaggcccactgagtgcaaagccactcagctaggtaagaagagttttgacacctccccatcctcctgg

SEQ ID NO: 199 - DNA, 2001 bp, Homo sapiens
ggtgttgttcttgaacctcccggtgtttgagagatggtgggctgccctgtcaagggcactggctggaagttgtggcttctactcctggctctatgtgtgatctgaccagctcatcaggctccacaattagctttctgcaatgtgggagtggattcttacaaaatgtagaactctactttctaccctgctcggggtctgcatgaaagatggcgggggtaagctttggctgcaaaatggtaaatatttcctgagtgtctactgtgtacagtgcagtgaggctggtttctgccgtacatggtatatagttatccaacagacactagctggcatgggctcttactgtgtgactttaggcgagttacttaacctttctcttgacgttccttttttcactttttttttttttttttgagacaaggccttgctggagtgacaagctcattaggttccacaattagccttctgcaatgttgtccaggctggagtacagtggcacaatcatagctcactgcagcctcaaactcctgggatcaagtgatcctccagcctcgggatttttttcacttttattattttatttgtttgtttgtttatttatttatttatttgagatggagtctcactctgcctcccaggctggagtgcagtggtgcgatctcggctcactgcaacctccgcctctcaggttcaagtgattctcctgcctcagcctcccgagtagctgggactacaggcacacgccaccacacctggctgatttttgtatttttagtagagagagggtttcgccatattggccaggctggtctcgaactcctgacctcaagtgatcctcctgccttggcctcccaaagtgctgggattacaagcatgagccactgcgcctggcacttttaaaacagagacaatacttacctcataggtttattgtgagagtggcaacagcttgcaatgggatgaggcacatggtaaggtgctcaatatgtgtgaactattataacggtcatagttaccattaagggaagacctatctgtgctacattaatgggggataataattactttattcgctgggcgtggtggttcatgcctataatcccaacactttgggaggctgaggcgggaggatcacttgaggccaggagttggagaccagcctgggcaacatggcaaaaccccatctctactaaaaatacaaaaactagctgggcgtggtggtgggcgtctgtaatcccagctactcgggaggctgagaattgctggaacctgggaggcagaggttgcagtgagccgagatcacgccattgcatttcagccagggcaacaagagcaaaattctgtctctaataataataataataacaacaacaacaacaacaacaacaacttcattcaaccaaatacattgtacatctcacctggcctgcccataagcaatggagacactgaaataaacacgattcctgccctcaaggtctaatggaggaaagagatgtacacagctattctagagggaaattgacatgaacagaagttctcatcctgtgctccaggatcacactggaggagggagatgctcccagagggtcttgggaaggctttccaatctggagctggaagaatgtttgcccaagagatgtacacagttattctagagggaaattgacatgaacagaagttctcatcctgtgctccaggatcacactggaggagggagatgctcccaaagggtcttgggaaggctttccaatctggagctggaagaatgtttgccagagatgaaaggaggggaagggaattccgggcagagggaagggcgcaggcaacagctaggaggcgcagatgcaggctgggtgtggagggtgcgagtaggctggagtacaccagggagttcccagcaggccactaggagccacccaggatgcccggcaacctcatgagtcagacaagcagggctgtgtctatctggaagtttccagccaagctaggtaaaacacatgg

SEQ ID NO: 200 - DNA, 2001 bp, Homo sapiens
gatccccggtgagaaagtgggggccacttctcttcctcaccgtcctgcctggggttccagagctgataagagggacagatttcctagttctgagatgcggggatctgctgagtcctccctcagggctctgggactcctccctctgctgacctgaggctccactcctcagaccatagccctctcctcattgacaccccaaaggcacagcaaagacacatcacatgggccaccgtgcctgaaaccacccagggctgaggtccccactgatctggactccaaggtcccttgagtcctttctcttcttcctccccttgactcttggctgggttgttctcctctaccacgccccgccgcagacctgagtcaccgtcccttggattcctcaggccgagtgaagagacatctcggtctgagacagaggtggggtgggcaccctgtcctgggatcccttcatgcctccctcatttcccagcagggcccgggccttccctctgctcccctgaagccgcattcttggtaccaatatccccttccctacgtcagcctcagatgccgacgtcagaatttgcgttcctggtcgccatgaccagggcttcccagggccgacatcaggggcagagccctccctgagcgtcccgacattgatgctggcagaacctaggcgcggctcccgccgaaccagccgggccctggtcacaccaagctctgactcccagagtcccctgcggcttaagcggcgggagagagtgggggcgggggtggggggggggtgacagcccagcctgagaagtctgccctcaggtggtccagagctggcagcagaggcagcgctgtattatttagggctttctatctgggttgggggcgtcctcagtcctccctcagcctctcaccttgcctcctcacagagcctgggcccgctctcttcagccgatctcaagcctgtccctcagaacgagaacctcgcttctctctgatccccgaggcgtaagttagtggcgtcacatccggtccccggggcttccctgggttgacagcaggggcggagtggattctgccggaactggggtgggagttggggtgggagtgagggtgggagtccgggtggggtggggtacgggtgaggatggggttggggcaggggtggtggtgaagatgagtgtggggaggggcgtggaggtgggaactgggatggggatcttggggctgtgggtcgtggtgggggtgggggtggggtctttcagtcatccctcagtgtctcgaccttgatgcctggaaaagcctggacccctgttttgtgctgaggatgtcttctccccttaaaccaggtctcttgactcggagtcttccaaggtggcagtgaggggagggttgtggtaggggtgacaacattgccagggtcttccagggctgacaagaggggctcagctggattctgtggcccctttgtgaggggtgggtggaccctcggtcctcactcaggagggtcttcctcctggcttttgtctctttgatgaagtgatgggtgggagatgctcagtttccgtcacctctgatcatttgcccagctgcatcccttgcagagcatgggtgcggttcccaggccagatggtccctggggagctgcaacacccgtgattgtaatgacctatgggagaagacgcacaccttcccaacggggctcttcccaagccacaagtagacttggcaaactttttgtcctaaagggtatattttctacaagaaagaggctgagcatcggggtgtggagaggtcctgctgttttgagtttagctgatattttatagtccaatgcagtgatgtctggttttctaaccctcattcctagtaaaaatatgtgttttaaatgtctgccaagtgtatattgaatacatatataacatttcatgaaagaatacctaccttacgctgtgtggttcactgcttattctatttctggtctattctttatattctattataataatttttctctttttt

SEQ ID NO: 201 - DNA, 2001 bp, Homo sapiens
tttatgggacaggatcacacacagggtggtgctactttctagagccaaatctcaagtaaggtgtccaggggtcagttgcaacagaaggcacattagccacagcagtctcagagaacactgctagaagaagaacctacacatggccatgcaatgaggctgaggggcaggcctaagtggaagcagaccaagtaaagaggtcagagagcccatggaaggagaagagggaaagacagttccaagagtaaagcagcctaggttaaggtgcagaaaattcagcatggatacttttaagagagctgtgcaaggctctccttacctaaaatcacactccgtatacagacatacccacaaacaatcacatcatgttctttgctcaagattgaacttgctgaaactgtgatgtgctactactcctaccgggggtagagggtagtctgctgctgtacttctcctctctggttactgatgcaattatctatagaagtataatgccgctctctgtaagcaattttaggtaaaatccatgggatattttcacagtgagctagcctacagagagggcaaaagtttctgggttcttaaagcagctgccgccaatgcaagctctgtacaggaggaatttcttttttttttttcgttttattattatactttaagttttagggtacatgtacacattgtgcaggttagttacatatgtatacatgtgccacgctggtgcgctgcacccactaactcgtcatctagcattaggtatatctcccaatgctatccctcccccctccccccaccccacaacagtccccagagtgtgatgtttccctacactccacctcatgcacaagcccaaatatatcacagcttctctacagaaatgcataattcactggcgaagttaatagacattggccaagaggatgatctcctcactatggaatcagctacagtcttaggtagcatgcaaactccagtctgactccaatcccttatatcttccatcgtaaagtattattgccaactttgtttctttccaatttttgtatgttctgcagtttatgtaaaggctagcaaaaacatgaatactttattatacatatggcaaatagtctcaaattgtaatgactcatttttatcctcttatactgttgtaaaattcagaacaacatgtattcactataggagtatgaaaattctttttaccaatgtacattttaaataacattgtggtaaagagaagctacatatttcacattctgattcaactcactgtatccaacgagtaaaagatcctagaaagaatcttacatttctaaaattcttatagactaagagtgagcgataggaacaataaaaatatttctgtaagtgctcttggtaaacagtcttaaagtcagaagaacacttatccaaaatgccctacattattgtgcaatcaaagtagataactttaacttcaaaaacaactcagtcttccaacccaattcttaaaactctaagctcttcacaaaaaatttttagccaatttaaacttatttctacactcagttatgtttaaaacaacataatgtttgcataattttttaaaaaacattctaactcaatgtttttatttatctaatattaattaccagatataaaatctttaaagtagcccaatatttttcaaaataatttctgcatagatgattgtcattcagtttataaaggtttatttataattttgtgataggaataagaaaagacaaaacctctgaaacaaactcacacacaaaagcaaaatacaaaacaaaacagaaaaaatacagtgaacttacttctgggaaaatggaagcaaggaatctagaggtatggtttatccactgagctccttgtttagaagaaagttattcatattttctcctggtctctaccatgtatctagaatttgctttaactatttgacttctgtgaaaagtttctgtgaagactgctttcagagtggaggttatgatctcaattctg

SEQ ID NO: 202 - DNA, 2001 bp, Homo sapiens
gtgttcctcttatttgttctcccagctgccaggtgaactcttgaactgcaaatcacattgttttcctctcctgcttggaatctgctccttggggcccactgtttgtggaatggagtccttgctgtgtctacccaaccctactccactcacctctctcctatgtcaacttactgccctcatccctggcctccactcattctcttcctaccctttgcccctgctgttctctctgaacaaagttcagctccctcgttccctgggtgctctagggttagggacaagttcttatttgcttacatgttcctcccacaaggcctgggaggccagctctgcatttcctagcttgcagcaggtatgttgtaaatctctccggcaggctgccatgcttttgatccccttccttcctgacctcaacctgacagatgcactgctgttattaccattttatggagaggatgtgggccttggaaagggaggagactttataagggtcatgcaaccacatgtagcaaatgattcatatgaggctttcaaaccaatacccccagcagggctgtgggggtgctggcaaggggccccatcctctgccccccggagtcctgcaagtgcccctagtgcggctcctctcgtcctgccttacctcttttccttgctctgctgccactgcctccttgctgttctgaatgctccaggcctccctgccactgccagcatttgctagggcatctccatctgcccagaacaccctttcttcaaatgtcactagggctttttgcattgaacctcatttaaccttaaagaggccaccctctttaaaaccccaagccccctcccccggcattccctgtcctcctcactctgctctcctttttctcttttcacttatccccttctgacatttgagatcatttactcatttcatatgtttttgtttacttccatctccctgctactaacacagaaactgcaagagggccgggatctttgtcactgtcatgccgtgaccctaacacttagtgcagggcctgatttgcaggaggtgcccagtgaagaaagccttgctcagggtctctcggagctgcggacagggtcatcatgggggcagccctctcctctggggtctcccagtccctccctgcccaaaaacctggctcacctggctcccctctgtcctgtgtccgcactgtaggccgggctggcctctccccggctgtaggccgcagcctctggcaggcttgtggtcctgcccttctctcctcccacataggggctggaacacagaagctgagagaaactgcttggccagctctcaagcccttgtctgctctcaagcccttgtctgctctcaagcccttgtctgctctcaagcccttgtctgggcttatggagctcctctccttcctccctctcttcctgcatgggtgtgacctttgtgcctgtcgctggggctgccctgggaggacttctgggaggttggagcccttacatctcaaagccagggattcctttgacactacttgtaccaaaaagctgtgtaccccagcagtgtttccaaagtgatatcctggtcttccctgatctttcactgtgtgcccttcaggcactcaagctgttaatatcacacgagcttaataggctcccctgagatggtcctcatgtgaaaggattgctctgtaaggtgtacgtttttcctctctccccatgtttctgctctaaaatatggcttgtcttatgcttagcatcccctgacattccctggaaaaatatttgtgaagcatccatctgtagtgtgtgggatccagatgagagctgggtcttagtgagaggaatgagaggcttagaggttccctccctgcataaccaccttggaaagtcagatgcggctccccattttgcaggtaaggaaacaggctcagggagggatgtcactttgtttgggtttgcacaataagtagattacgtaacacatttgtgtaagactgggaaagaaatcaaggggcc

SEQ ID NO: 203 - DNA, 2001 bp, Homo sapiens
tcctatggctaaagtgctcaaaggggaaaaaggagtttcaaaaatgttcttggaaataccatctctcacgaattcttcggcctctgctgtcccaatgtcacttgtctgagatgtaaacagaggagttctgagaaagaagctgaacttgcatttctccctgtttctatttgttccaaacttgtggcatttctaacaggatgaagcggaagagaaagggaaagagacaaaagtgtagaaagatggaagatcccagctgcaaatggccatttgcagttagatggaacagctgctgacgttcagggaaatgcatgtctctcttcagatgggaaggagcagtggaaaggggtgacgagttcctggctggccaccaatcatcccatctttctgtgccggttcctcatctggaaagtgggagtgatacttgtgcttgcttttcctacccacaaagattattgtgagagctataatacggtgagatacagaatcctgcttttaaaaatacaaagcagaatcaagatgtcaataataaggatagtaattgtgttagttatctgcaatcatctattatagctagtcgtctaggatcctggatcgttctcctggttttactacagttttggatcagctcacccccaaatcccttgctgaagggtggagctctgtcagccatgggcagggaaccacttcctcttgcctttctactttctgtctttcaaacatgcccagggtctttgcacttgctgttccccctgcctggtacctctctcctgtggcttgccccagagctgatccttgtctttgtccacttctcagcgaggatggcacttcagggagcccttcccttactatcgcagagagagcaggccctccccagtcatgtccaacccagaactctgttttgttttcttcatagccctagcatcacagaaaatcaccctgtgcattcatggatgtccacgggggcaagggctttgtgttgcttaacccagcatcctgaaccgtgtttgttgaatgaatacagaaccccgtttgctctgggagagcacagaaaacagtcttctatcatatatcatagccagctgcaaacagcagatggcttcccatatcccagagagtaagaaccagagagagagagaaagagagagagtttgggtctttctcctctgtgcctgctctctccagagaaactggaggggtagcagttagcattcccccgctggttccaccaagcacagtcaaggtctctaggacatggccacccctcacctgtggaagcggtcctgctggggtgggtgggtgttagttggttctggtttgggtcagagacacccagtggcccaggtgggcgtggggccagggcgcagacgagaaggggcacgagggctccgctccgaggacccagcggcaagcaccggtcccgggcgcgccccagcccacccactcgcgtgcccacggcggcattattccctataaggatctgaacgatccgggggcggccccgccccgttaccccttgcccccggccccgccccctttttggagggccgatgaggtaatgcggctctgccattggtctgagggggcgggccccaacagcccgaggcggggtccccgggggcccagcgctatatcactcggccgcccaggcagcggcgcagagcgggcagcaggcaggcggcgggcgctcagacggcttctcctcctcctcttgctcctccagctcctgctccttcgccgggaggccgcccgccgagtcctgcgccagcgccgaggcagcctcgctgcgccccatcccgtcccgccgggcactcggagggcagcgcgccggaggccaaggttgccccgcacggcccggcgggcgagcgagctcgggctgcagcagccccgccggcggcgcgcacggcaactttggagaggcgagcagcagccccggcagcggcggcagcagcggcaatgaccccttggctcgggctcatcgtgctcctgggcagctggagcctg

SEQ ID NO: 204 - DNA, 2001 bp, Homo sapiens
ggtccccgaggggccccgaccgttggcaggagccgcggggttgcctctgatgcctggacaaggagggagaagcaatgggagtcattccatcccgaggtgccacccattcctatctccggacgcctcttcccaaatcctgcggcctctgacaaatgcctcacaatgtctgggctgacctcgaaaattcaggtacgaccggtcccattctacagcggagggcaccgaggctcagagaggccaacggggctgttttcgggtaacatctacctgatccagatcccgcacgtgccttccccctgccctagcctccgtcccgcatgagagagggggtacttaatcaccgggcttcggtgccaaagcctgcgtcctcgggcactgggcggacacccaggccccgccgccagccactccagaaccggcccaaaacacggcggccgtccatctcccgagccaggacagcgcgcatgtgcgtccagaggggcagacccgcgctccagccagcgcgctgattggtcagctggagtgtcaatcggaatggtgaccgggcgggactcagagcctgaggaggcggggcttttcctccgcccaggaaggaaatgcagaaatgccttcctatgcttttagggagagcgctgtagcagaagaatttgatctgatcaaaaaacctgtcttagcaaaaaacctgaagccgcctttgcaaaaattatattacaaaattatgacagcgaaagagatctgatctaaccaacccgcagcttgcctttaacctctaaacggtccttggtcattcctgggcttgggccaagctaactttgggagaaattagttcgtagtctaaatgataattgcccttcccccgaactaaactggcttcatagagttaatgaaaggccaccaggttaggagtagtagagggacctgaattctgctaagggttagaggtaactgattaccagcggttattccggaggtcacagtgtttgcagcttccccagctcacgattgtccaacctgagattggcctttggagatgtctttccaggcttttgcatttctgacggccggatcaccccacccggaccctcaactcttggctctactagtcctgtggcccctcgaagtcaggagatcgagaccatcctggctaacacggtgaaaccacgtctccaccaaaaatacaaaaaaattagccgggcgtggtggcgggcgcctgtagtcccagctactcgggaggctgaggcaggagaatggcgtgaacccgggaggcggagtttgcagtgagccgagatcgtgccactgcactccagcctgggcgacgagcgagactccgtctcaaaaaaaaaaaaaaaagaaaaaagaaaaagaaagtagaagaacacagaaaaaaaagaataatataatgactgcccacattcctaccatcagaattagtcattgtgaaacaatatatatatgtataggtttcctgtttttcttgtttaagaaatgaaagggataaaagtaaattcccctttactgcgtgcttccagttttcactccctccccagtctccactttgagtttggtgtgaacattttcctaatgtgttttggtttttgttttttggagacagagtctcgctctgtcacccaggctagagtgcagtggcccgatctcgactcactgcaacttccacctcccgggttcaagcgattcccctgcctttgcctcctgagtagctggaattacaggtgcggtgcccgccaccacacccggctaattttttgtattttagtgacggggtttcaccatgtcgcccaggctggtctcgaactcctgagctcaggcaatccgcccgccttggcctcccaaagtgctaggattacagacgtgagccactgtgcccggccactaatgtgtttttttgcttgctctgtcatccaggctggagtgcagtggcacaatcacagctcactgcagcctcaacctcccaggctcaaacgatcctcccacttcagtctc

SEQ ID NO: 205 - DNA, 2001 bp, Homo sapiens
ctgcaactctatataccaggcaactagttgtcaaatgctaattactgtcatcatccttaaagacttgggcggcaatgtttccattattttcacgatggggaaacgcaggttccagaggttgagcaatggccacgaagccacactgctaggacctgagggtgtgagcttagctgcaacctccctctttggcccctgctcataccaagccaggtgggtgaactgagagtccaccgagacgcccagcacttcacagcccagcttgcggaagtcctctgcacggttgctgaacgcgatgatctcggtggggcacacaaaagtgaagtccagagggtagaaaaagaggaccacgtacttccctggggaggagggacacagaggagttagggcccagcttctctcatgcacggccccgcttctacctgggccccctccgggcggcgctcacctttgtagtccgacagcttcacctctttgaaggcgccatcaaccaccgctgtggccttgaagtcaggggctggctttccgatgcgcgcgttaccggaggccatgactgaaagctgagacccccgcccggtcagtgcgcccgggaagacactttgtcctcccaacccaaggtcgcgctgcgtgctgggccctatgactgagtcagcacggcggaggcgacagcactaaccctcaccctcccgggtacccggtctacgaggcccggaggctgccggagacccgctctcctgaccagagcacgagtgacccagccactgccaccagcaccccacgagcgagtcggccggagggcggtgtcgcaagcccccagcccagaaggagacagtgaggcccgcacgagcagggggcgggaagtcgggcgatcaggcctgcgctgcaaggctgtggcctcggtttcccccgcaaggacaaaggaggccactatagcggatggaaagcaaaggcggccagcactaaagtcaggatcgggccaaagcagcctgaagggggtcctcccgccaggtgcactccgggtgttcatacctgcgtgggcaaaggctagacgcacggacgatcacacgcgtggacccgcgttctcagcgccaagtgagccctgggccgcgaagccttttatgcccggcccgaaccaagacgtgcacggcgcgggggagggagcggacgagcggagcggtcgggtccactgcgcccgcaggtgcgggggagcgtgcggccgggtccatgcgcctgcgggcggcggggggagacgcgttgccttcggccgggaccactgcacctgcccgcgtgggtaatgcgcccgccgcagactccgcgcacgactccgcctgggagcgcgttgggggccgttggagtccagcatggcgcggaccccgggggcgctgctgctggcgcctctgctactcctgcagctggcgacccctgccctggtctaccaggactatcagtacttagggcagcagggcgaaggtgacagctgggagcagctgaggctgcagcatctaaagggtaacctcaggcggcggggatgacgatgcccaaaggctccccgttgccctacttctcctgctggctgcaaagggaccttcctcggcgggctgcaaatctccacccagctccgcactggaggcgtccccttgggctggttacttttctctaaggctcggtttactcttggatatattgaaatagatactgctttaaagcagctcccaggggaagagagggtgtgggcaggacactgaagctgtcactcggggataggtgccaggaagggcagagacagagaaagaagggggcgggcaagcttgggaaatcagagagggtccgattgcagaagacccgtgaccagtgcagcgagaacgacaagcgaaagtcccatatggaaagatttggggaaagaatgtccaaggaagagggaatagcaaatgccaaggtgaaaagtcctgaaggagaaatggaaagaggtcggtgaggtgtggcagagacatgggtgtgggaggg

SEQ ID NO: 206 - DNA, 2001 bp, Homo sapiens
cccactgggatcccctgcccactagacttgtccccatcctgaggcagcctgttcctcctgacccccgaaagcttcccacccatctcccttcggcctgaagccagcccccctcctctctcccttctgtttacctcccaggtttgggctctattttctactccttccacctcttcctgagtaacaaggacactcccttcatccacaccccatgcctcctgccttgcttcctgtaccttcaagtagttggaagaggtcatgggtgagaattccttctccttggcctagtctggcccaggtctggagagaaggcaatggagatgtgaagtggaggccaggtgtggtggctacgcctataatcccagcactttgggagtccgaggtgggcggatcacctgaggctaggaatttgagaccagcctggacagcatgctgaaaccctgcctctactaaaaatacaaaaattagccaggtgtggaggcatgcacctgcagtcccagctactcgggaggctaaggcacaagaattgcttgaacccgggaggcggaggttgcagtgagcagagatcgcgccactgcactccagcctgggtcacagaatgagactctgtctcaaaaaaaaaaaaaaaaaaaaaaagaaattgaagtggaaaatgagctggcaggggtggtcaggcagggggtgagcaatgagggctcctgagctgaggaagcaggggcggggggaggatgggggctagggagtcctcagaagttcccggagatactccctcactggacagagtggggttcccgatgacgttacttgaggcctgggagacggcctctgcccctcactgctcctccccctcctaaggtcaacatttatgtgtcctagacctagagaggtcccaggacacgccactgtcccgccttccccattgcccgccccactggccagtccccacgcccacacacccaaggctgccccatctggcgctgattatcctgctgctgccgccaccgctgctgctgctctgcaaaattcagctgctgcctctgtcttgaggaccccagcgcctttcccccggggccatgctgcctgcagccacagcctccctcctggggcccctcctcactgcctgcgccctgctgccttttgcccagggccagacccccaactacaccaggtaggtctcttggcatcttccagacagctccagtgaacttcagacccccgattcctttggggttatgcctggagatattttgggttcccttgctgacacaccactccccagattccccaccgcctcacccttctctggcccccaaatcctccccatctgctgcaagaccttggtctcctgcaggccaccactgctcccattcccccaccttcgtcctctggtctcttaagtctccccacccacccagatccttgaccccccatagaacccaagagagagccaagctcctgaacttgcctggtagctggattccaggaaggaattcgagagggattgtagggggaggagggggcagtgagtggagacttgggagtcagaatctgaagtagtttggaacaggggcagggctaaggtgtggagctcgcggagcctggcctccaggttcaatcagagggccatcgccactgcagctccctccatctctccaccccctcccccccaccgcctcgtggctgtgtccacatctggaagtagttaggcggctcctgtccctggactgccaggcagagtgaggcgggagtctggaaggggggccgaggaggattccgggaattcctgttggggtgggggtggggactcttggggttggagggatggtcgagttgccaggcccacttttcccttatctttgtgggagtctgggacaccccagccctgtcctggctggtggaagctgccccaggtctctcaaggcctttccatttcatcccccagcttccctccttggagtttcatcctcatcacctcatccccatcttgccagtgctcccccgtcc

SEQ ID NO: 207 - DNA, 2001 bp, Homo sapiens
tgactgagaaggaatagctgatacagaggggagggaatttctgaaggagggagtgaatcatcaacaccatcacatggcttagaaaagtggaccctggatttggcaatgatggacataatccaggtggactttgagcctcctcctgcccccagtggccactgcattctgcagccctgaactctacctctacctctgcttttcttctgcaaaatgaacctcttatatacaattctgtttcctacatcagttactaaactttgaggcatttgagccaaggggacccgtactcgcaccaagtttcatcacaggtgtctaatgtgattacaggggcagcacggtgccctgcacgggaaagtactggggtcagagagaccagagttcaaatcgtgactctttacccctgcgatccctttggtgcaaatgacttaatccgtctctgcctcagctttctccactgttaattgggcataatggacacaacaatggtgaggggctcttgcaggaatgaagactaggatgaggatgaggatgagggatacgtccataaagcccctgcacaccttccatttcctgagcactcccaggaagtctttgctgaccacgccaaattagattatctttgcttagccattaggccgctgccctatattgggaggcaaatatacctctcagcttcggtggccaggtcccctggcacccacttagtcctcagtctgcacctaaaagcaagccttccgagggtcttgccttgcaggtgtagctcccaccccccacgctggggtggtccggggaccctctgtttcctcagtggtggggaagggacccgtgggttcgtgggtgctgaccgacgaatgtgcggaggagggcgcgccccagcatggggggcgggggtgcttcttaggatggcttggaggggggcgcctgcagggctggacggcggacgggggacactgcactttctgatgcggtcggggaagccgggagtcagcctactggagtccgctgccatccctggccagaagcagccagcgccaccccaagctccccagcccctcgcccgtgccggggccgccggggtccgagttaggggagagtggggggcggccaccgctgcaccgcgcggccttcgaaggactgccctgcggcgccgcgttcccccgaggccaccccctcccgccgtcaacactccccggggcgggggctcacctgtgtggtctgggtcgcctccgaggccgagtccctcgttgccaagcccagcaggcctcgctcccccgtgactgcaccgccggcccgctccagcttcccgagtaccccacctgtctccccggcttccttccggccctttgtgttttcttctcttgctccgccccctcgcctgcccgctaagcacttaagctggtgaagtcaccctactgccacctggcagtcactttcgctgttggtggcaacccaggctggaacccagctcttgccggaacccctgccccatccttgaggcttgtcccgacaccgagtaggttctagttcctttgtgccttccaaatgactcccttcctccagcaatcacagccgcaacagccatgtattgcgcccccgccctaggccaggcgtgggcatcacctttgcactcctttctgcagccgtgtgtggtgcccactgtgacctgcacttcacagacgggtcatctgagttgtgcaggccactaagggagcctggagtcaaccccaggcctgcttggctcccaggccttctctttccattgctctttgtcggtcccctgctgcctcctgtccctccccaactctgacttgtggagggaagcctttgacatgtgggggtacctttaataccgatatcacttgttaccggagaggggtcccgatccagaccccaagcgagggttcttggacttctcgcaagaaagaattcggggtgagtccgtagagtgagagcaagtttattaaggaagtatagaaaacaggccgggtgcggtggctcacg

SEQ ID NO: 208 - DNA, 2001 bp, Homo sapiens
tgaaggatggggaaatgattccctatatactttgcttttactaacaatcatgtatgtatatatataaatcatacaaggaattctacagatttccaggcagattttatttagccagggaatgacctaagaagtgcatctctgttttcatctgcagaggctaaaagtttattgtgatcctcaagtcactacatccctaatactcagagaattgacaggaatgaaaagctaaaagatctaggaagttgcaaacgttacctgctgatggaaatctgttgtctggcttcgtttcggcagcaaagtgtgggcacaccatcaggggtgctagatttattccatcgggattgcggaagtcagctccagtgatcggctggaaaatttgggtaagaggaggaggcggggatagaaatgctgagagaaggaagggtagggaggagggaaggaggcagtggatttgcagtgtttctgtgtgtatgcagcatgtgtgccagggtagggaattactagctatttagtctcacaagactgtctttggtggcaaataacaagacctcaatatctattgactcaaataggagcttactttctcttttggtgagtaaaaggaaggacacagccctagtctctcccatccttcttcttcaatatctttattttaatattgggggattatatcttttcctacccctttcctgttaggatttacctctccaacattgaaacctattttcatatttgtgtctgactctcacctctgggatgcggtaatgaacataaaaggctataaaagaataatgtaatggaagccataggctccagaattctttgtatttttataagcttgttggttgcaagttgtcattacttccaggcagaatagttttcccagcctaatgtggttttgctgaaggaggatatgcccagaaagtccagatgaaactgtctttaaaagtaaccaaaaacagcagtcctggttatttatgacagcacttgaatcaatgccgtaagttctgatggactcacatgtgactctgttgctaaaactctcaggtggtgggatgcccgataaacatcatctgtctcaattaaaatccccaatctaggacaagtcacatgagcttctccaaatgcttccccaacatccaagcatgtgacagatcataagaatctagctgattcctcaaaccacaccccaaacttcattttcttccataaacagatgtggggaaggagaaaaaaaggcttcttggaatagaaaggataataaatgggatctgtaatagactcagtttctagctggattccatgcataaaccctcaaaaatacaggcttatgcctctggattttaagagctgctaacaggctaggcgcagtggttcacgcctgtaatcccaggcctttgggaggccaaagcatgtggatcacctaaggtcaggagttcgagactagcctggccaacatatagtgaaaccccatctctactaaaaaaaaaaaacacaaaaattagctgggcatggtggcacacacctgtagtgccagctacttgggaagctgaggcaggagaattgcttgaacccaaggggcagaggttgcagtgagccaagatcgtgccactgcactccagcctgggtaacagagcgagactccatctctcacacacacacacacacacacacacaaaaaaaaaaaaaaaaaaaaaagttgctaatgaaccaacatagtctcctagagggaaaccaggtccagacttgaacagtttaaatttcactcattttaatccagaaaaggacatactgggggaaggcatccagatgtgccctagtggttttcaagtcccgactcttcccctctctcccaagaacccaggcaacgcccaccccaaaacccccacctcatggtcagagcattcctgctgatgttactcagaatgtgtcaggatcaggaggacaagtgaggggggaggcgtgcaatgtgatcagaaccctatgatttctgattcctga

SEQ ID NO: 209 - DNA, 2001 bp, Homo sapiens
tattcctatgtttccaaatagaaatgaaaattctgaaatttcagataattcccccctccaaatcccaattctcaatttagcttttctgagccttgtccctaccgtgaaatcttactagctcaccccgaaacgccggggtcttggtcctctgggggactagtgagtcctgctcctctcggctttgctccgtccctgccccggcccctccgcgggagtctggggtcgcggcccggtcaggcgccaagccggcaggggggcgcggtccacttgaggccacagctccccaggtccagggctcgggccatccgcgctgtcccttccgcgggctcttgctgttcttcgccaggaggctttgcactccgggccgcatgggtcctggataaggacctcaagaggttgtagtggcgccccttcgctccggcgtactgtctgaaccctgtgcccaaagagggctgtggcagttcctgcaccaaccgcctggagcccatacctaagccctctgggcacgagggacctcctctcccccgctcccccctccccccatcccaactccagccccaaaggaagcaatgcgcgcgtccgagagcaggagcgcgcgtggctgaggaaagaaagggaaggcaaccgggcagcccaggccccgccccgccgctcccccacccgtgcgcttataaagcacaggaaccagagctggccactcagtggtttcttggtgacactggatagaacagctcaagccttgccacttcgggcttctcactgcagctgggcttggacttcggagttttgccattgccagtgggacgtctgagactttctccttcaagtacttggcagatcactctcttagcaggtaggtgccgcagaccctgcgggttaagaggtggggtggggggcagtgcttgccaaggccctaaactgggagcgctgggtgaggggaacaacccactttggagggttctctgagagatagatacaccccatatcctgggcccagctcgtgcacacagctggaggtccagagacccagtcccctctgctccgtcagccaagttccaagaagttgagcagagaccctctgggagcctggcggggtgcagcggcctcccctgcggggcctgtcacccggccggcgcgtgcaaacgcctctggcgcctctctgcgcggagggagataagcgtctgagccagggaaagcgcgggctaaacccgcctcgccggggcccctgcccgccctccgtgccccgcccgggcggtgcagctggcccgggtgctcacgctcgactctctttcttcttttccagggtctgcgcttcgcagccgggatgaagctggtttccgtcgccctgatgtacctgggttcgctcgccttcctaggcgctgacaccgctcggttggatgtcgcgtcggagtttcgaaagaagtgagtccgggcagcgccttcccccttgctggtacctggcaggcaaggggaactgaccgttggtcccgaaggtctagaagtgaatgggagcagggacaggcctgggcgtcacctgaacgcacgcgaatcgggtctgcttgtgttttccaggtggaataagtgggctctgagtcgtgggaagagggaactgcggatgtccagcagctaccccaccgggctcgctgacgtgaaggccgggcctgcccagacccttattcggccccaggacatgaagggtgcctctcgaagccccgaagacaggtaactacgccctgtgctgtccagggacgggagggaaggaaggtgtgcgggaggagttctctgtctccactcccctggcccgggggatcgtcggggctggaccgcagctcagatggcgcgagcagtttccagctccctctggctctagaatggctcccgttcccggtgttggggccaaagctctgcttgatggggtctcaagttgcctttcttccccctccccccgcccgcagcagtccggatgccgcccgcatccgagtcaagcgctaccgccagagc

SEQ ID NO: 210 - DNA, 2001 bp, Homo sapiens
tatgaaaaacatagtgaagtgaaagcaaacatctgtccagaagggaatagaacaataaaaatgcccaataataaatttaatttgaatgtgaagaactaatatgaagcaaataacacagtcattgtgagggatacataaaaagacttgaggaaatgaggagtggctttttcttggatagagaaattctaaaatagaaagatagatattgtccctaagttaagatgtaatttaaaaataatacctagtgtctcacctggaatattttagcacctgatcaaatgattctgaagttcatctgaaagacaaatgtgtcagagtagacagaaaaggaaaaagcaatgagggagggcaagcattatttcgcattacaaatataattaagctacagcaattaaaatagtatactagtggtacaggtacaaacgactcaatggaagaaaattaaaataagagagttacatacaaaattgagtggggatgtaggtgatgataaaagagccacttcaaaacagtgggtgaaacagggattcaacaaatgatgtagagtccctaccacttaattttgttcagatggatcaattttttttttgtttgtttgttttgttttttttttttttttgagatgaggtctcactttgttgcccaggctggagtgcagtggcacgatctcggctcactgcaacttccacctcccgggttcaagcaattatccagcctcagcctcccgagtagctgagactacaggcgcgtgccactatgcccggctaattttttgaatttttagtagagatggggtttcaccatgttagccaggatgatctcgatctactgacctcgtgatctgcccgcttgggcctcccaaagggctgggattacaggcgtgagccactgcgcctggcccggatcaatgattttgaatgtacaatcatgcaacaaaaactatgaatgtcttatcatattggagtgggtaagatcattgcaagcatgacagcagactcgcgaaggcacaagaaaatgatttatgaaagcagatataaaatgggtattaacatatataatatttatttctatgatggcgaatttgtccgaaacacattgttttcttagagtaacctgggcctcatattctccaacgccattggaagtgaaaggaaggttgggcgggggaaaaagaaaagtgctttgtaagttcattttcgttgtcagaagagcttgtggtttaggttctccacagaggcaggaaaccttcagtcacgtggcaaataccccactgagtgcgcatgctgcacagatctgttgctgcgcatgcgcctttctgcccaccttctgtctaggcagagctctgcaaggagaggttgtgtcttcgttctttccgccatcttcgttctttccaacatcttcgttctttctcactgaccgagactcagccggtaggtctgcagagtggtcttcctggtaatttagttgtgagtgaatgtgtggaggagccagcgggcttaggacaggtcccgtggcacagtccgtggctttgagggaaaagggcctcgcggtggtcctccgccttcccccaggtcctgatgcaggcgccatgggccggtaatcgtggctgggctggaacgagggaggaaggtaggccgtggagggggtagatcgcctgaagatggtgcgagtcctgggggtgggtgctgttagaggtgtctgagtcccgaaaacgcctggaactctctgagggaggacagtttctggactcttcgttagggatgcgagaagggatggtgtgggtggtaaggaaggggccaggaaactgggaacgctgtgggctggtgactacggccccgaggtctgtagagtgcctggcagaggtgtccagtgaggaacataaccttaactctgtgtcgcaacttctcatctcagccatggacctcatggaaaggaatggttgaggctgtgcattccaacaaaacttgatttttcggggagtggggaggaggtggtggtggc

SEQ ID NO: 211 - DNA, 2001 bp, Homo sapiens
cccaaacatgctttactggaacttacgccctggcataagggccacaacaaaagagagagcgaattctctggcttgctgactccttggaaaaaaccggtagggatttttttattaggcaaagcacaggaattgacgtcagaggcaggatgtgctgctgggcaaagcatacgagaagtggggtatgcaggtcagcattacttggttgcaatggttatcttgaggaatgggccaactggtggtctggccagtggcaacaaggctgtaaatcaattattcagcattccttcccaaggtgggacacccggcaacattgtttatctcctaaggccagttcctggaattaagtgaaaggatgactaatggacatgttgtcagtgaggtagtggtgtgggttttgtgaccagtgggaatgcacgaaagaatgctttagcggggagtgagctgaagccaagccccatccctactctgtctcaaagtgagttcagaaaaggggatttaaagaattctttttttttttttttttttttttgagacagagtcttgctctgtcgcccaggctggagtgcagtggcgccatcttggctcactgcaagctccgccccccgggttcatgccattctcctgcctcagcctcccaagtagctgggactgcaggtgcctaccaccaagcccagctaattttttgtattttttttttagtagagacggggtttcaccatgttagccaggatggtctcgatctcctgacctcgtgatctgcccgccttagcctcccaaagtgctgggattacaggcatgagcctccgcccccggccttaaataattcttaaaggaagtaaagttaactttgaaagaactatcaggatttggattgactgaaaggagtggggaagcttagggaggaggtgcttgccagacactgggtcatggcagtggtcggtgaagctgcagttgcctagggcagggatggagagagagtctgggcatgaggagagggtctcgggatgtttggctggactagattttacagaaagccttatccaggcttttaaaattactctttccagacttcatctgagactccttcttcagccaacattccttagccctgaatacatttcctatcctcatctttcccttcttttttttcctttcttttacatgtttaaatttaaaccattcttcgtgaccccttttcttgggagattcatggcaagaacgagaagaatgatggtgcttgttaggggatgtcctgtctctctgaactttggggtcctatgcattaaataattttcctgacgagctcaagtgctccctctggtctacaatccctggcggctggccttcatcccttgggcaagcattgcatacagctcatggccctccctctaccataccctccacccccgttcgcctaagctcccttctccgggaatttcatcatttcctagaacagccagaacatttgtggtctatttctctgttagtgtttaaccaaccatctgttctaaaagaagggctgaactgatggaaggaatgctgttagcctgagactcaggaagacaacttctgcagggtcactccctggcttctggaggaaagagaaggagggcagtgctccagtggtacagaagtgagacataatggaatcaggcttcacctccaaggacacctatctaagccattttaaccctcgggattacctagaaaaatattacaagtttggttctaggcactctgcagaaagccagattcttaagcaccttctgaaaaatcttttcaagatattctgcctaggtaagtctgttgtctgcatgtctccccactaatgtgagtcatatagatggagtctcagggcacgactgggttttgtgtctctcgttgttgcttcacagcccttttggcatcacccatttatttaactaggataaaaacgaatattggtatagcgattccacagtttacaaagtgcttttgtatccactgtctcacttgatcaag

SEQ ID NO: 212 - DNA, 2001 bp, Homo sapiens; identical to SEQ ID NO: 182

SEQ ID NO: 213 - DNA, 2001 bp, Homo sapiens
gccaggggatggggaaatggagtcaggtcacagccatgcctggaacctgcactaacgccctccccatcccagaccacagagagccagccaggaaagtcattccacacccatgtttcagaaaacattcaaggtcagatcctttttacctaaatcctgctttcatttgggtttgcaccaggctcatccccctcctccagcaagccttcctggatttattcactcagtggatgccattccactggggcaaacctttccggcacctctcctgacctaaggcacagtcctgccccaatccccttaccctcctacccagatcactcccaagttaaccacttgagtgatccacagcaggctgtcagccccggggactggcttccagctcctgcccaagcccaaaacaggaaatggggctccaaggctgaagccttgatcttaacacaggcaaggttaaatgctgccaaaataaatgatttaaaaaaaccctggcccactgaggtggaagtgaccgggagataatctcctgctgttcattaaccatgccgctgcctctacctcaggcagggcagggcaggcaggcaggccactggaaggctgactcaagtgtttccttccttaacttagaagctcctcctatcctcagaccagggacacaggacacagagggacaggatgggggtgggggggtgacatctctagatagtggagggcatcttgagagcaagaggcagatgccccctgcagccagcttctacgtttagcggggtaatcaacattcaacctcgccctggaggaagacacctggctttccttcccccgtagggcaaggctgcgcctctccactcccctgccggacaggcgcccgcagttcccctcccctttgaggaaagggaggcaggcccggaggttctaagaacggccccaagatttcccttcttgaggtttccatagccccaaagcctctgaatctcattcctaactttacttgacaatctactcctaacgttcgccaccacttaaacctcccccaggcctcccagggaccaccagtggctgagccccacacagcgcacgtttgggaagcttgaagacgaccccgaaatgcgaagaaccaacgtttctgtcggtttacgcccttaagcacgaggcaagggctcactggcccggctgaagttcggggtcctgatcccccaccagccccgccctgttaggcctttagccgtttctgtgcccccgcgccccctactcactgcgtgtgctgggcgcggcctgcgccgaagacctcgtccagcgccaccgtggcctggcccaggaacttgtcgacgccgatgagcgagcggtgcatggtggtgagcaccagctcgcaggcggcggcggagctcgcggcccagggcgccgggcccgcgtcggcctcctgcgcccgcagcaggccatccagggcccccggcggcagctcgaaggagcactcctcacgccactcggggcagccgtgcgtcttctccaccaccgacgtactgtacttctcgcggcccacctggatcaccgtgtacgcgtcgctggtgctgcccgctcccgagctcttgccccgcagcccgcgggcccgcagcaccgtcacctggacgtgcgtgggcagccagcgggaaggccccgccgccggctccgcgccccgcaccagggccatggcggagaagcggggggcgaggtctggccgagccggtgcagaccccaggacctggtgacaaacccggccgcggcagaaggcggtcaggaaccaactctgagcgccgccgcagctgcgggctgggctgggccgggccgaccggccgccgcctccccgcctcaccgggccgctggccgcgggccgcgccgcgacgtcagcgccccgccccctgccccgccccgcgcccgccgccccaaccggcgaggcggtgcgacagggatgcgggcacgcagctcgcgccccggcagccgcagaccagcccggagcgggaaggggcggtgcgc

SEQ ID NO: 214 - DNA, 2001 bp, Homo sapiens
gtttgctgttagttatctcatacctaaagggctattatcattattaatatttttattttttgagccagagtttcgctcttgttgcccatgctggagtgcaatggcgtgatcttggctcactgcaacccctgcctcctgggttcaagtgattctcctgcctcagcctcccaaataactgggattacagggatgcaccaccacacccagctaattttgtattttcagtagagatggggtttcaccatgttggtcaagccggtcttgaactcctgacctcaggtgatctacccgcctcggcctcccaaagtgctgggattgcaggcgtgagccaccacgcccagccctattattatttttgacaacaacagcaaagatctgcccatccgactgcaggaatttaatacacagcctttgtcagtggtagaaattctatcactgtatgactcatgagtacataacaatgtgctataccatacttcctctttgagggataaagcccctgcactctaagggaggatttcattatgcatttctcttatatcaccagaaaattatttagtggaagacagtttaatatttgacagctttgaaccatttttttttctctttcaaccagtctttttacattgtctgcttaaaaacaggaaaccacatatctccaccccagcacaacccaccatccaccagcacagaccgtatcattttggatctacagtctttttcttccaagcctggccaggtgcctgctcctactttgcaggaaacccctccaggcactcccggtgcgtgttctcagagcccttctgagaatattcaggattgcacttcctgggctgcagcgcaccctgtaccctccttttttaagcctcctcccctgagtcactgctacccttccctcctctaacttccccttctctgcagcctccttagcaatttcttatcataaagtttgttttaaagaagaactgcccctctaaaaaaataataaactgtcgagggcggtagccccgtctagaggtttccgactttgctctgtgcccacctgaagcctccccagcccgaggtatttgaacttacattcagtgcttcacgtctctgtccggggtgcctctgtctctcaactctgacttctgagtactggcagttagaacaggaaggaagtcaatgagaaaaaaaaatgttttagacctacttgaagaggaaaccctgggctgtctcaaatcctgttcctttgcactgcctatctcttgatcagccaacttgagcagcattcttgtcaattaatggcacatgtgcgagcatgacagcccgtgtgacgtggagatgcatgaatgtacacgcaagagtgtggagcctatagctatgggtattatgaataattgtgagggtatttctgataatcaggtcattgcagtgcataagcctatggaggtaaggagcctgaaaccagtagcaataaattcacatcggctttactttgtaccactcacttctgttcccactgggtccccgccagcttcattatcaaccagatacgttgaaagtttgctggcagtgaatgtagagtgctaagaaatgatgattgttaactgcatatactacggtcctaaatttaaaaatgtgtccttaaacagatttttggagttttgttggttataatttcaagacgtagctttgtattgcactacaaacagtcaaatattgtttttaatagttttggctgatttttcttcaaatgaaacaaacaagcaagcgaaagtactgtctttgatgaaacctaggaaagaaattacaaaaggaagtaaaacttcattaattcatagtcttccaactcagaatttgtgacaacttaacaagtgccaaaatagactttgtaccatatttgaggaaatggtttatgaagtaatttaaatgggaacaagtctcggaggccatgtttaaaccatctgaggaacagtttttaagcagccaagtcacattcatgctgtatattcccgtagc

SEQ ID NO: 215 - DNA, 2001 bp, Homo sapiens
tatttagtgtgtgttctcagcctcaccattgacttgctgtgttaccttggacaagtcactctcccactctgacctcaattttcacatctctaaaaggagactgctagactagacggtgagtgaagtcctgctgagccctccgagttcattctcccttgtccagtcagtccttccctcccaccctcccctctgggctccctctctggggccctctcccccaggggaggcctgctccagccccaggggagctgcggccctggctcctgttcccagtcggagtgaccagccctgggcctccttcccagcttcggccctggctccatgtggcccatctgagaggctggcctcaagcccggcggcaactccacatttctgtttttccttttttttccctctttcccggagttaacaagaagcagatgtggcgcacgatggttggagaggtggggggaggaagggggaggccggaccgccagccagacaggggggaagggaggggagccagcagggaggaggaggccagggcccgccccacagccactctcgcgcctccgaacagccacaggggcaaagccctgtcacccccaggatccggtcatcagggaaagaggacagggagaccagaagagggccagctgggacgagggggcggacgcccaggaggcaacttctgagacgcagctcctgagaggggcagggaccaggcgcgggaggccagagggggcacagagaacaaaccccctcagaagtgaagaggagagcggaaggaaccgagaggggacggacaggagctgaggaggaaagaggaggggagaggggtcaggccaggcagccaaggagaagacgtgtggccgggggctatcagaaggaaactgggacggacgggccgggctcgggctgtcctgtggagcagcagcatccccggggccggcagaggcgccagtggctgggcgggatgagtctctgagggccactgtggagcgccccgccatggccccccgcaccctctggagctgctacctctgctgcctgctgacggcagctgcaggggccgccagctaccctcctcgaggtttcagcctctacacaggttccagtggggccctcagccccggggggccccaggcccagattgccccccggccagccagccgccacaggtaagagtctggatcccagcccgaggcttgggtggtgaggaaggggtcagaatgccacctctgcctggctctctgctgtgtccgctaatgcagggcagagggggagtgagaagcccatccatcaggtggctcctcacatgcccaccttcccacggggccttttccaggaccccctgaacacatatgttccttgggtcacctagggggtccgctcccacatgccccagggaagctgccctgaggggagcgggccaggccttcctgccagctgccctctgtatcctagccccagagcctcagggagaacttttgtccccttctagggctcccttgaccccatccagtaatccataattctgacaccactccagctctgttccctccagccctccagccagacacagatcagaggccggagtgaatggggccaggagtatagaactgcaatccccaagaggggcaggctcatcaatggcttattctctacttaaccccaagctggcctgcccgtcttctcctggaggggtggggtggaagccagtgttcagatcacccacagggcccgggctggctatgcgcacacagcatgccaatctggggactgagacaggcccagaaagggacacacatgattgtgtacattcagaggctccccgggaccccaccaccatgaggattgactgctctgtggttttccattctgcgcgtctccccatcttgaaaagtctttatctgctcctgctacacaggaaagcgtgggggatagggagggtggcgatgccaaagcccttgctctcactgatccccaagggttctgcatctttggagccttcttcagccc

SEQ ID NO: 216 - DNA, 2001 bp, Homo sapiens
ttttgaattatttgccatgaaagcctttcggtgtcccctccatcccccccaaaaaaaagaaagagaaaaagaaaaaaggccctagcactactagaactcaagcagaaattcacattctcccagcactcacgtaggataagaaatagaattctaccccaagtccaaaaaagagtctggcccaatatcaaaatagactttacctccaaagcagtttgatatttattaaacactacctttttctcttcattaacatctcttgcctcctggtaatttgccctttacttttcctctgtttctccagctttgaaatcacacgccattttttttaagacaatgtaacaatgtgcaagatattttttgttgttgttttttgcacttttgacataccctatccccaaacacaccccaaaccaggacactgtggaattaatacagtttggaaattgggtatagtttagtgactcagtgttagttaagatgctggcaacagatgttggcaaaggctatggaatgagtcccaagaatgacaaaccttacattactccaaaggtttgaaatccaggatttctctggttaatatggttaatagttccactgctggttttaattgtcttagagtggaaggaggtgtcatattttcaggccctagtaatctcttccttcagaattcttctgaataaaaagcttagcaacaaaatcactagagttagagtgctatgccttcttgatttaatactaaaacatggaacaaatgaaaccactctggcctttttccaacctctacagatccctcattcttatacttctagtccgtcttttcttcctcataacaccccaattccctttttgctctttcttttcctcctcaaatcatatctctgcattgtctcgctatgtggccacccttgatctccagtgacctccaaagggcaaaacgcccataacaagacaacaactgcagtaacaatccagtccaaaagtatttgccaagagtttattccgcggttagcaccaaactctccatctattttgccactgcaaacagtgaacccatagttccccaaaacaatcatagttcctcatgcttcactcttggcagggctgaggacaggaaagaagtgaaaataaaatcttccattactaccctcagtctagccagaagtataccccaccacacaaggagatggcagccaagtcttcactccctcagagtttctcctcaggctcctcactcacctaactcttccattgtcctacatccaagatgctcttgtctcacaggccgtgaggaacagctttggcatgtgctgaagaagaggaccgcaaaggaactggatgagacagcataaggcagcctctataaacctttttggttcctgtatttgcttagcacttcctctcttttgatttggtgagcttttttttttttttcataggttacacagttttaaaaatgtgactgacatagaaaggtagatagtaagatttgcattatggcagtgcattggccagggtgataaagattcaaaattaattttgcaaaatttgaggacctactctgtgccaggattcatcttggattcatgggaatggcagacttatacttgcgttttttccaagtgtaggtaccccactctgagcatctttcttttttcttttttttttttctgtgaagctgggagacacagaggcatagtctccttcttaaaatttgcatcagagttgaccagcatccatttcattttgttgtcttatttattcagaaattatggttatctgctttcctgcatggaacgtaaatccttaaaagtagggcagaagtggattcatgcttggattctcagggcctggtttactcagtggtcccaaaagcaaacatctatagggtggtcacaaagaaggaagccaggtttgacaccagatgttaaactgtgtgcatctcaattaagcataatccctgcctagatacactgccttatagcaatttctttggaaagtaac

SEQ ID NO: 217 - DNA, 2001 bp, Homo sapiens
ggagtgggctgcatgtgccgcgcggggagcccgcccgacttcggggaggcagcggggcgccgtggccccgcggctcagctccctgccgggtcgggtgtcccagccctgcgacttccggggcccggcggctcctcggggagctcgtccacaggcagcctccggggcgagagagcgcgcggcgcgggcgccgggacggtccgcggcacgcgggtggagccgccggcgctcgggctcccgcaggggcggcgggcggggcggggtgaggcgggccggccgccctcccctctcctccctcccgcagcccgccctcccttcctccggtcccgcagccaattaggcggccccctcgggcgggaggcgtggggcgctgcataaagcggcggggagctgccaccccgggagcgggctgcgcagtgtccgggccgagccggtgcgccgcagactagggcgcctcgggccagggagcgcggaggagccatggccaccgctaacggggccgtggaaaacgggcagccggacaggaagccgccggccctgccgcgccccatccgcaacctggaggtcaagttcaccaaggtgaggcgggcgcccctcccacccgacggccgcgggcccctgcgctgggcagccagactcggggcggcgggggcgagggagcagcgggccgggggtccgccgggcgccgccgagacccgagcctccctgcccgggcgcggctctccagaaaccgcacctttcgcgggacgtctcgggcgggatcgcaggcggcggggctcggcgctgtgagcctcgaagccgggaaaccgaggccgtccagcgcccgcgcggggccgggccgcccgcatccctgcgaggccccgacgtgtccctgcgctgcccgctttgatcgcgagtggaggtgcgcgccgcgttgactcggagcgcgctggttttggggagctccctccccaaaaacccctgcgatgatttccagggtggacgagtctggctttgagagggtcggagggtccgagagcgccaagagagagtccgggaggcagatggcggagctgcagttggaagactggggaagcgggtcctccgagcaaagcgcagcgggcgccttccgtgcttagtttatacccggctcagcagcagaacgaggaggagtaggaaggtgctcgcttactgacgaaacctccccggagcccagaggtcccggtgtttacacacaaggcgcgcgctccggaaggtcgcagacctgggcctttgggaagcggtgggtcgcgaggccgcagaagcgcacggttctctgcctcctcgctctgtgggctgggaggctacagcgttggcagaacggcgagattacctgcccggggcagaaaggaataaaagaagatggtgaggcctccgaggcctctgagctggggctgaggagggccgagagacccgggtccgggagcgcgacccgatggaggacacccccaggctgtctggcgtgcgcgtctgtcgtgggccggtgctggggcactttagaggcacaggccaggccgagagccattgccttaactttccccacctttggcttctcttgatttttgcttttacccatagcccccaactccagctgacaccgcacttagagaatttcctcatccgttttactttaagaagtagcaattatctttctcaaagaattttaatcaagcggagaaagttgcagggcactatgtgtggccttcagatttgggtctcttggcacttaattcccaatgtcggctgagcctctgatcacaagacatttaaattaactgttttcagaaggctctgtaatcaacaaagacacgtcgctgtaaactaaacgctggaaattattcttaatgtagttttatttgtaatatcagcttcagagtgaaaattccaacacgaattctgggaagagtgaaaatggtacccgaattttgtaagaccctaaccccaggtgcttggctgctgctactgatggttaagtggggaggttgt

SEQ ID NO: 218 - DNA, 2001 bp, Homo sapiens
aggcaggagaatcacttgaacccgggaggcgtgggttgcagtgagccgagatcgcgccgtggcactccagcctgggcggccagagtgaaactccgtttcaaaaaaataaattaataaattaaattaaaggtatgtgaatgcaatctctcgctgacaaaatatctttatgtactgtagatcttggcaaccttagcatacgattttgttttctttccttattatgttaaaaactttcccattttcacttaaaggaagcaagcactttacagcttctttttggcatatttcaatcgccagcattgctacccttgtactttggggccattattaagtaccataagggttaattgaacacaagcactgagataccaccaggacagtcactctgataactgcgaccgctcctaagtgactaaggtacagagagcgcagccagccaacagcatggggactcaggacaaagggaggattaccatcccagggcagggctgactcaagagcttatcacactactcagaagggctggcgacttaaaatttatgtattgcctatttctggaattttccaattaatattttggaacctcaggtaactgaaagcacagaaaggaaaaccctggataagggaagactactgtagttggagcagagaattgcaatacctaaaatatttgccatctagtctttcacagaacaagtttgccagctctagttttagaggagcaaaatgaaagatagcaaatttctcagctcaccagttcctaaagttgcgagtgcatccagatgattcagtgcagctcagtatgttccactctgggccacaagggaaagcgaagctggagcttccttgtctccacctccgggagaaatttgtccagagaccggcagcactgcttccgcaggcgaacaacgccgcttgctcttcaaagtcctttctgaggccagatcagaaggtcccactccttttaagccccttattgcccaatcatttttccagtcacgtgtctgaagcagtttatcctgattggattttcagttgttgtccagcttgattgccaaaagttttcctccaattgctactcaaaggaaagaatgagggtggggcttcagggctgctttccaggttgattgagctgctgatgggcatcctccctcctctctctcttgttcagagtctaccactttctctgcagtgtgcctgggccctcagctattggggatgagaagggatgcggagtgggaggcagtccacatgtctgttccaacaaagctcagaggtgagcgagttaaaaagcagatgtctttccagtaggagccccaggggacagggtgggtctcacagcccttgaagaccgagctcccgtaggagtttccaggtgtccaaacagggtcctcactgtgagtcttcaacagctttctccaagcagcagcccactcccttcttaatccatttccaggggtcaaaggtctatcctggttccctcccatgccctctcacttccttccctctgtctctccctcctccctcctccttcactaccccctccctggaaccttctcagctgaatttcaggcccaccctttcactctctttacaggctttgcaacacagattagaaagcgggggagggggagcctcaagagtcccttgacaggtaagaaatatttatatttccacccccattccaacgcaatgcaatcaagtctcaaaattaaaacgaaaatcatttgcaaattaaaatgttctaaagtaccatctcctgataaaactttaaaaatacctttctgggccggatgtggtgactcacatctgtaatcccagcacttttgaaggccgaggcagcagatcacatgagcccattagtttgagaccagcctggccaacatgatgaaaacctgtctcttattctttataaagtaaaatacaaaaatttcctgggcgtggccgtgcgtgcctgtaatcccaggtactcgggaggctgaggcagaataatcacttgaa

SEQ ID NO: 219 - DNA, 2001 bp, Homo sapiens
gaggctaagccggcagatcatttgatgtcaggagttcgagaacagcctgaccaaaattgtgaaatcccatctctgttaaaaatacaaaaaaattagccagacatagtggcacacatttgtaatctagctactcaggaggctgaagcaggagaattgcttgaacccgggaggtggtggttgcagtgattcgagatccgtgccactgcactgcaacaaagcaaaagcaagactctgtctcaaaaaaaaaaggacaaggacaaggtcagaggcaaaacaggaggaacagaggaaggaagggacaaagagggaccccaatgcagactggacgagtgcagagggcagggatggaaagtcacgtcctgagctgtttgctgttgcagaatctcagcagctgggccataaagaagggacttgacgctgtcccagtctctacacttggcacccttgacctctctcatcccacctttccctaggccgaggtcacacctgctttcattttgtttttttttgttttttttttttaagacggggtctcgctagattgccctgacgggagtgcagtggctattcaagggcaccatgatagtgcactgcggcctggaattcctgggctcaagcgatcctcccaccccagcctctcgagttgctgggactacaggcgtgtgcccctgcacccagctgcgcccgtttttgtcctcctgtctggcttctcttcatgatccccacccctttattcggcctcaggggaaccccgtggtctattccgtgagaccgagcagagttttggggagccccgactcttggatctccctgctggctcacctttctctgttcggtctccaaaccagaccagcaatggccccaggaagaaggtggcgctgggagaagttccctaagctatgtatgcctacctgtccccttcctgacttttcggttccccaaacccgaggctggacctacctttaatcagatccacgatgcgcagctcgaagggaatgtcgttcttcttggcaaagatgtaaacagcgcggcagggctgggacagcaggtccaggtacagctccaggcccatagtggggaccgaccgacctgaccggaaaccagagggagtcagcaaactccagtgccaggactcgggtctcggacctcagaccacgcccctgcggctacgcaccccgaccgctgtgccccattgggcggcacggacgcctgggacactctttagtccaattgcgggcggcgggcggcctgtctcagggctgggcccgcctggctcctgtgggatcagggtgggtggccaagagaccgttgtctctcagggggacgcacagagaggccccagtcccatccctttcgtgcctggctcgctcatttcacttagcataatgtcttcaaggtgcatctgtgttgaagcgtgtgtcagaatttcccaccgtccgcattttagaggaggagacagacactgaatgattatagacccagaaagtctggctccaaagtccatcctcttggctcaacactgcagagtccttggtgccagcaagtggcaaagcctcagggcttacgttcagtgttttgtggtcactcgcttcactgcagtcgcactccctggaatgtcacagccttggtcttcctgcccctgccagcacagtgccatctatcgcattccctgggtttatttcttcctttctccaggcctgtgtttctccatctttaaaatgacagggaagctagagccctccagagtccttgcaagtccacccccctggattcatctgctgagccgtaagcccaggcctgccccttgctgccatggagcctgtcaaatagtatcagggtcacatgtcattttctaaaacgaatcccactgaaagtgtgtcagccaacgcataatgttttctagagaataaggaacatttctgcaaataaagtggaagtaacaactgttttattttatttagatagagtctcactctcttgcccagggtagaatgcagtggc

SEQ ID NO: 220 - DNA, 1147 bp, Homo sapiens
gggggaggagccaagatggccgaataggaacagctccggtctacagctcccagcgtgagcgacgcagaagacgggtgatttctgcatttccatctgaggtaccgggttcatctcactagggagtgccagacagtgggcgcaggccactgtgtgcgcgcaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccacgagactatatcccacacctggctcagagggtcctacgcccacggaatctcgctgattgctagcacagcagtctgagatcaaactgcaaggcggcaacgaggctgggggaggggcgcccgccattgcccaggcttgcttaggtaaacaaagcagccgggaagctcgaactgggtggagcccaccacagctcaaggaggcctacctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcagggcacactgacacctcacacggcagggtattccaacagacctgcagctgagggtcctgtctgttagaaggaaaactaacaaccagaaaggacatctacacgaaaacccatctgtacatcaccatcatcaaagaccaaaagtagataaaaccacaaagatggggaaaaaacagaacagaaaaactggaaactctaaaacgcagaggccctctcctcctccaaaggaacgcagttcctcaccagcaacagaacaaagctggatggagaatgattttgacgagctgagagaagaaggcttcagacgatcaaattactctgagctacaggaggacattcaaaccaaaggcaaagaagttgaaaactttgaaaaaaatttagaagaatgtataactagaataacc

SEQ ID NO: 221 - DNA, 20 bp, Artificial Sequence
  Synthetic Primer
ttttttgagttaggtgtggg

SEQ ID NO: 222 - DNA, 23 bp, Artificial Sequence
  Synthetic Primer
tctcactaaaaaataccaaacaa

SEQ ID NO: 223 - DNA, 13 bp, Artificial Sequence
  Synhetic Primer
gggtgggagtgat

SEQ ID NO: 224 - DNA, 18 bp, Artificial Sequence
  Synthetic Primer
cgagcgcggctacagctt

SEQ ID NO: 225 - DNA, 20 bp, Artificial Sequence
  Synthetic Primer
ccttaatgtcacgcacgatt

SEQ ID NO: 226 - DNA, 18 bp, Artificial Sequence
  Synthetic Primer
accaccacggccgagcgg

SEQ ID NO: 227 - DNA, 22 bp, Artificial Sequence
  Synthetic Primer
tgtcgtcggaaattggcagtat

SEQ ID NO: 228 - DNA, 22 bp, Artificial Sequence
  Synthetic Primer
caaagaccagctgcaaggaact

SEQ ID NO: 229 - DNA, 17 bp, Artificial Sequence
  Synthetic Primer
tctttcctgtgatcttc

SEQ ID NO: 230 - DNA, 18 bp, Artificial Sequence
  synthetic primer
cgcgagtcgaagtagggc

SEQ ID NO: 231 - DNA, 23 bp, Artificial Sequence
  synthetic primer
acccgattttccaaatacgaccg

SEQ ID NO: 232 - DNA, 21 bp, Artificial Sequence
  synthetic primer
tgtgtgtgagttgaagtaggg

SEQ ID NO: 233 - DNA, 26 bp, Artificial Sequence
  synthetic primer
acccaattttccaaatacaaccatca

(19) United States
(12) Patent Application Publication
(10) Pub. No.: US 2013/0316931 A1
(43) Pub. Date: Nov. 28, 2013

(54) MARKERS OF MELANOMA AND USES THEREOF

(71) Applicants: Luca Sigalotti, Maniago (PN) (IT); Michele Maio, Roveredo in Piano (PN) (IT)

(72) Inventors: Luca Sigalotti, Maniago (PN) (IT); Michele Maio, Roveredo in Piano (PN) (IT)

(73) Assignees: CENTRO DI RIFERIMENTO ONCOLOGICO-INSTITUTO NAZIONALE TUMORI-AVIANO, Aviano (PN) (IT); AZIENDA OSPEDALIERA UNIVERSITARIA SENESE, Siena (SI) (IT)

(21) Appl. No.: 13/979,309

(22) PCT Filed: Feb. 1, 2012
(86) PCT No.: PCT/EP2012/051663

§ 371 Date: Aug. 12, 2013

(30) Foreign Application Priority Data
Feb. 1, 2011 (EP) 11152864.2

(51) Int. Cl. C12N 15/10

(52) U.S. Cl. CPC C12N 15/1072
USPC 506/9; 506/16

(57) ABSTRACT

The invention relates to a method for determining prognosis, predicting or monitoring response to therapy of patients affected by melanoma through the evaluation of the methylation of different genomic DNA sequences, including the Long Interspersed Nucleotide Element-1 (LINE-1) repetitive DNA sequences and/or the concomitant methylation profile of specific groups of genes defined as methylation signature.

### FIELD OF THE INVENTION

The present invention relates to the methylation of genomic DNA sequences, including Long Interspersed Nucleotide Element-1 (LINE-1) sequences and genome-wide gene methylation profiles. More specifically, the invention relates to: i) the use of low levels of methylation of LINE-1 sequences as a prognostic and predictive marker in human melanoma; ii) the use of multiple gene methylation signatures as prognostic and predictive markers in human melanoma; and iii) the use of combined LINE-1 methylation and multiple gene methylation signatures as a further improved prognostic and predictive marker in human melanoma.

### BACKGROUND OF THE INVENTION

Cutaneous melanoma (CM) is characterized by a constantly growing incidence and high mortality rates in industrialized countries, representing the leading cause of skin cancer-related deaths worldwide (MacKie et al., 2009). Only surgery, in the initial phases of disease, may have a curative intent for CM patients; otherwise, advanced stage CM is characterized by a substantial unresponsiveness to conventional therapies (Bhatia et al., 2009). At present, the unique predictor of the 5-year survival of CM patients is the clinico-pathological staging of the disease, which defines overall survival (OS) rates ranging from 95% to 7% for stage I to IV patients, respectively (Balch et al., 2001; Balch et al., 2009). American Joint Committee on Cancer (AJCC) staging of CM identifies 4 stages of disease, stage I to stage 1V, and respective substages, which are characterized by a progressively reduced survival of patients along with the increase in the stage of their disease (Balch et al., 2009). In detail, localized CM has no evidence of regional lymph node or distant metastasis, and is classified as stage I or II. Substaging reflects increased tumor thickness and presence of ulceration and/or mitoses, and identifies the following categories: stage IA, IB, IIA, IIB, IIC. Ten year survival rates range from 93% of stage IA to 39% of stage IIC patients. Regional metastatic CM is characterized by the absence of distant metastases, the presence of regional lymph node metastases and/or in transit/satellite metastases, and is classified as stage III. Substages IIIA to IIIC reflect an increasing involvement of regional lymph-nodes and/or in transit/satellite metastases. Stage IIIC is the more aggressive, with a 5 year survival rate of 40%, as compared to 59% and 78% of patients with stage IIIB and IIIA disease, respectively. Stage IV CM is the most advanced stage, characterized by the presence of distant metastases, and is substaged to M1a, M1b, M1c by the location of the metastases and the level of serum lactate dehydrogenase. One-year survival rates are 62% for M1a, 53% for M1b, and 33% for M1c CM.

CM patients within the same clinico-pathological staging category often behave extremely differently, and the current lack of established prognostic molecular markers greatly impairs physicians ability to identify CM patients who will evolve through a highly aggressive rather than through a more favorable course of disease (Jennings and Murphy, 2009). Thus, the definition of prognostic molecular markers that enable predicting survival of patients within the same clinico-pathological stage of disease is a mandatory requisite to boost the current clinical management of CM patients, providing physicians and patients with more efficient clinical, follow-up and treatment procedures.

In mammals, DNA methylation, occurring at SC-position of cytosine in the context of CpG dinucleotides, represents a major epigenetic mechanism in controlling gene expression, chromosome X inactivation, imprinting and repression of endogenous parasitic sequences (Esteller, 2008). Alterations in the genomic DNA methylation patterns represent a hallmark of cancer, and include both global genomic DNA hypomethylation and gene-specific hypermethylation. Each of these apparently contrasting events are thought to play a major role in tumor development and progression.

Global genomic DNA hypomethylation (i.e., overall reduction of the 5-methylcytosine content) is a frequent molecular event in cancer and has been observed in neoplastic cells of different histotype (Gama-Sosa et al., 1983). Genomic hypomethylation might contribute to cancer development and progression through different mechanisms including generation of chromosomal instability, reactivation of transposable elements, and loss of imprinting (Esteller, 2008). Substantial decreases in the 5-methylcytosine content in the genome mainly reflect the hypomethylation of repetitive genomic sequences. Among these, the Long Interspersed Nucleotide Element-1 (LINE-1) family accounts for 17% of the human genomic DNA, is capable of transposition, and its methylation level may be utilized as a surrogate for the overall level of genomic DNA methylation (Yang et al., 2004). In accordance with the proposed role of genomic DNA hypomethylation in cancer biology, preliminary studies have reported LINE-1 hypomethylation to be associated with a reduced overall (OS) and cancer-specific survival in patients with colorectal and ovarian cancer (Ogino et al., 2008; Pattamadilok et al., 2008). However, these studies did not investigate the role of LINE-1 methylation as a prognostic factor in patients in specific stages of disease, but rather looked at the patients' population as a whole. Besides, no literature data is available on the prognostic role of LINE-1 in CM.

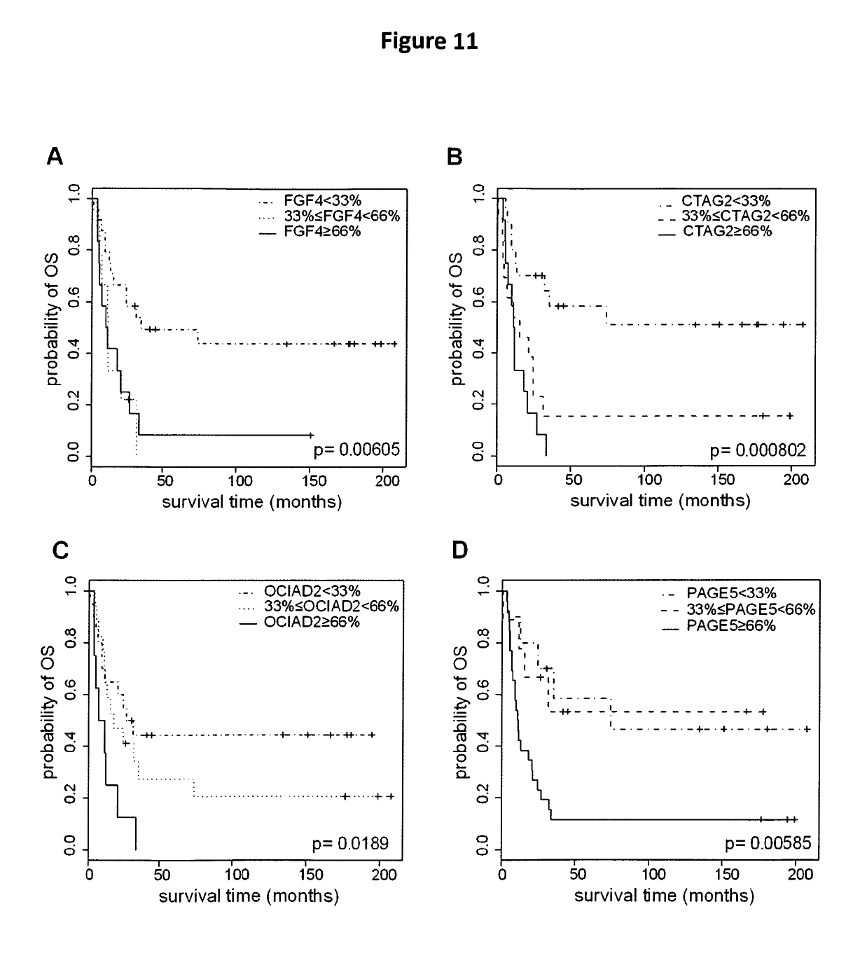

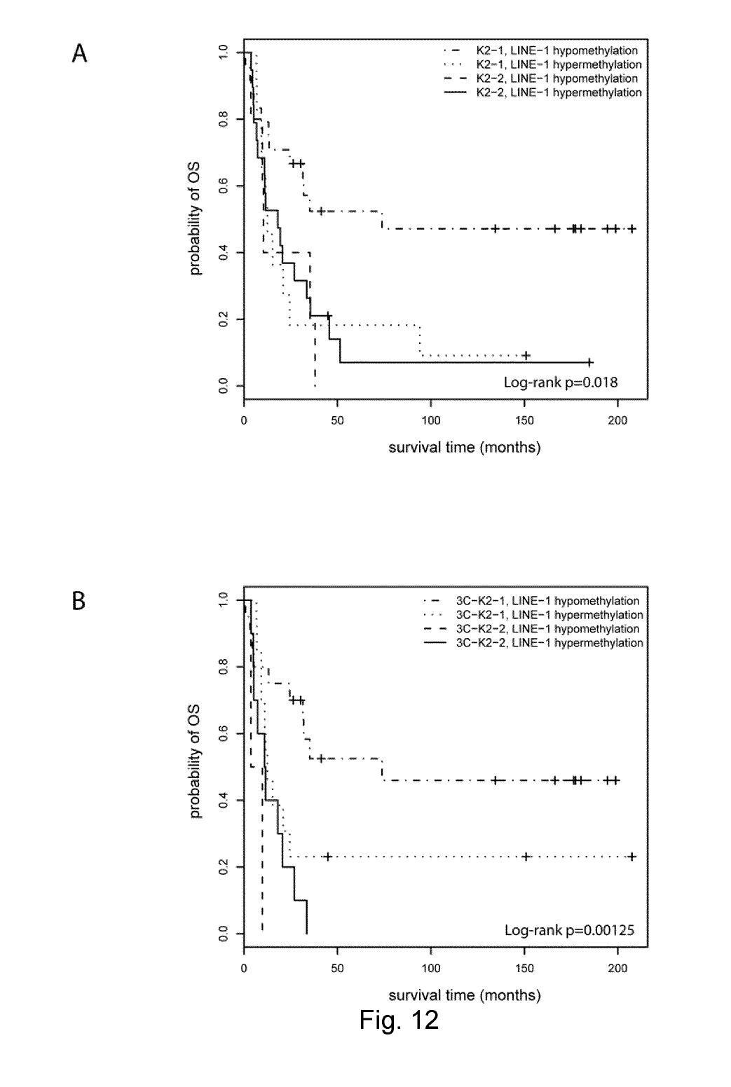

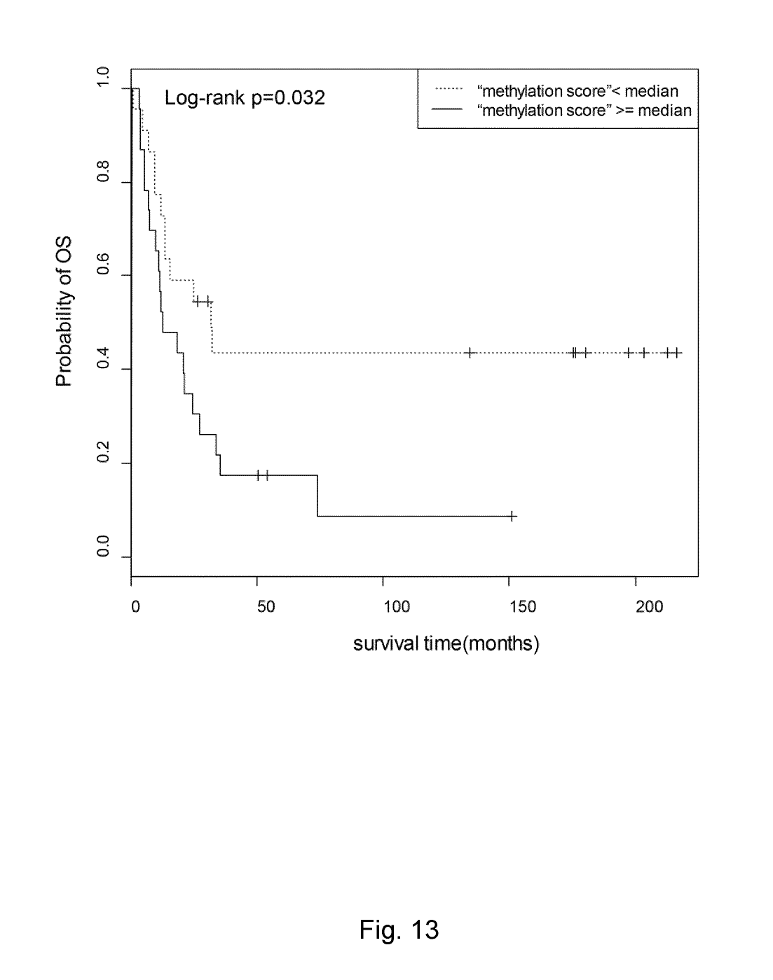

In the application WO 2008/134596, the methylation status of LINE-1 in breast, colorectal, esophageal and prostate cancer as well as in CM was investigated. No significant difference in LINE-1 hypomethylation level was observed between primary CM and adjacent normal skin (FIG. 29), nor between different stages of disease other than between stage I and stage 1V (FIG. 31). A significantly higher LINE-1 hypomethylation of metastatic CM vs adjacent normal skin or primary CM (FIG. 30), and of stage IV CM vs primary CM or adjacent normal skin (FIG. 31) was observed. The application does not disclose any correlation between LINE-1 methylation level and survival variables (OS, cancer-specific, or disease-free survival) in cancer patients. In addition, it does not provide neither suggest evidence of the existence of molecular markers able to predict different survivals of patients, in general, and, most importantly, of individuals within identical clinico-pathological stages of disease. Further, the data reported in WO 2008134596 would indicate that patients with low LINE1 methylation levels have a lower survival rate.

By contrast, the authors of the present invention examined the prognostic role of LINE-1 methylation status on survival of CM patients, in particular at identical stages of the disease. The authors found that within identical clinico-pathological stage of disease (e.g., stage III or IIIC), CM patients which neoplastic cells have a higher LINE-1 hypomethylation survive significantly longer, and a higher LINE-1 hypomethylation level represents a positive, independent, prognostic factor for CM patients.

Besides the overall hypomethylation of genomic DNA discussed above, the aberrant gene-specific hypermethylation observed in human malignancies might dramatically impact their biology by affecting different key cellular pathways involved in cancer development and progression (Esteller, 2008). Along this line, initial evidences in CM have demonstrated that this malignancy is characterized by a profound epigenetic deregulation, characterized by the transcriptional silencing of over 50 genes through the aberrant hypermethylation of CpG islands located within their promoter regions (Sigalotti et al., 2010). The inactivation of these genes affects diverse cellular pathways including cellular proliferation/differentiation, apoptosis, DNA repair, extracellular matrix interaction, metastatization and immune recognition, and is likely to play a relevant role in CM development and progression (Sigalotti et al., 2010). Accordingly, initial studies, conducted on very small cohorts of patients, have examined the prognostic potential of the methylation status of single or limited panels of genes in CM, showing an association between the methylation of the Methylated IN Tumors locus 31 (MINT31) in tumor tissues and disease outcome in stage III patients, and a negative correlation between presence of circulating methylated DNA for RasAssociation (RalGDS/AF-6) domain Family member 1A (RASSF1A) and Estrogen Receptor alpha (ER-α) and survival of CM patients receiving biochemotherapy (Mori et al., 2006; Mori et al., 2005; Tanemura et al., 2009). Despite these initial evidences, no study has investigated the genome-wide gene methylation profile in CM patients and its prognostic/predictive potential. Thus, the authors have utilized a whole-genome approach to identify genes or gene signatures which methylation status has a prognostic/predictive role in CM, paying particular emphasis in identifying multi-gene methylation signatures able to discriminate survival of CM patients at identical stages of disease. With this approach the authors were able to define distinct methylation signatures that were able to sort stage III CM patients into categories with highly significantly different survival rates. Even more important, the gene methylation signatures the authors defined enable to identify patients with significantly different survival also in sub-stages of disease (e.g., stage IIIC), which currently represent the upper limit of our ability to segregate patients into groups characterized by different clinical outcomes using the tools that are at present available. In this context, the concomitant evaluation of both the extent of LINE-1 methylation and of the gene-methylation signature(s), proved to have a further increased power to discriminate survival of patients as compared to each marker alone.

In consideration of such arguments, our invention has a significant industrial transferability to the clinical setting, where it will provide a key contribution in guiding the daily clinical management of CM patients.

### SUMMARY OF THE INVENTION

LINE-1 sequences account for a substantial portion of the genome and are usually heavily methylated in normal somatic tissues. Reduced levels of LINE-1 methylation (i.e. LINE-1 hypomethylation) significantly contribute to the well-known global genomic DNA hypomethylation occurring in cancer tissues. Besides LINE-1 hypomethylation, neoplastic tissues are characterized by a genome-wide alteration of gene methylation patterns that appears to profoundly contribute to the neoplastic phenotype. The inventors developed a method to predict survival in CM patients at identical stage of disease through the evaluation of: i) LINE-1 methylation status; ii) multiple gene methylation signatures; iii) combined LINE-1 methylation and multiple gene methylation signatures.

Assessment of neoplastic cells from CM patients within the same clinico-pathological stage of disease demonstrated a significant association between higher LINE-1 hypomethylation and longer OS. LINE-1 hypomethylation represents an independent factor predicting a more favorable prognosis of CM patients (Example 1). LINE-1 hypomethylation can be used as a surrogate marker for the expression of different methylation regulated molecules, such as tumor-associated antigens, that are relevant for cancer therapy (Example 2).

Assessing with high-throughput approaches the genome-wide gene methylation status in neoplastic cells of CM patients at the same stage of disease, the authors have defined multi-gene methylation signatures that identify groups with significantly different OS within stage III and IIIC patient cohorts (Table 9, Example 3). In particular, the differential methylation pattern of: i) signature A: MGC29671, GJB5, KCNK16, IGLL1, ANXA8, OR12D3, TUB, CST11, TMEM10, CRHR1, TRIM40, GAS2L2, FOXN1, FXYD4, KRTHB2, FLJ33860, S100A9, FOXI1, GUCA2B, CYP2B6, IL8RA, CYP8B1, WFDC8, and SLPI genes identifies 2 classes of stage III patients with significantly different survivals; ii) signature B: FLJ25421, LAMC2, Clorf36, MGC29671, GJB5, KCNK16, ANGPTL3, IGLL1, DEFB4, STK38, SEMG2, ANXA8, OR12D3, PREPL, IL17F, DCLRE1A, FLJ20581, PTCD2, CX3CR1, TUB, UBE2E1, CST11, IFT140, TMEM10, SLC22A8, MLN, DIRC1, Clorf64, CRHR1, TRIM40, TNFSF13B, GAS2L2, FOXN1, CYP2B6, ACSL5, SERPINB12, APOBEC4, TNNI1, Clorf42, FXYD4, PPAT, PSD4, SCN4A, KRTHB2, IL8RB, UNC45B, C1orf201, SFTPG, LOC124751, FLJ33860, ANKRD1, FMO2, ARSG, GJB4, S100A9, LRRN5, MSMB, HLA-DOB, R3HDML, SPINK5, DLG5, PALLD, WFDC12, WDR20, FLJ32771, PKHD1, FOXI1, DEFB123, KRTHB3, GUCA2B, CYP2B6, BSND, NALP10, NR5A2, KRT25D, IL8RA, APOD, CLEC4F, THRB, KRTHB5, CYP8B1, WFDC8, SLC13A1, C3orf27, MGC35206, C3orf63, DARC, SLPI, SLC2A7, CPA3, MIS12, KRTHB4, MRVI1, MIA2, CYP2A7, ANGPT4, SLC44A5, and CST9L identifies 3 classes of stage III patients with significantly different survivals; iii) signature C: SLC18A2, GJB5, SLC6A18, IGLL1, ALOX12B, WNT10B, TUB, S100A9, CRHR1, TRIM40, SLC6A11, FLJ33860, FGF4, S100A9, MGC35206, PCDHAC2, and GRM4 identifies 2 classes of stage IIIC patients with significantly different survivals; iv) signature D: FLJ25421, ANXA13, SLC18A2, CTAG2, GJB5, KCNK16, SLC6A18, STK38, PVALB, OR12D3, ALOX12B, HAO2, DCLRE1A, WNT10B, TUB, UBE2E1, IFT140, SLC4A7, S100A9, DIRC1, CRHR1, TRIM40, SLC6A11, TDRD5, ACSL5, APOBEC4, FAAH, CD1D, Clorf201, FLJ33860, ANKRD1, FGF4, FLJ39501, S100A9, LRRN5, GLT1D1, IL28B, AVPR1B, GJB3, MGC35206, PCDHAC2, GABRG3, MIA2, CTAG1B, UNQ9433, GRM4, and CEACAM6 identifies 3 classes of stage IIIC patients with significantly different survivals. Furthermore, the concomitant evaluation of whole genome expression and methylation profiles allowed to identify 2 multi-gene methylation signatures of 51 and 73 genes that were constituted by the sole genes displaying a significant (p<0.05) correlation between expression and methylation. These latter signatures identify groups with significantly different OS within IIIC patient cohorts (Table 10, example 3). Specifically, the differential methylation pattern of: i) signature E: DENND2D, CTAG2, PGCP, KCNG1, FAM107B, FANCE, FAM38B, FOXF2, BGN, MAGEA10, SPATS1, PRRX1, MLSTD1, PDZD4, HCP5, OCIAD2, CSAG1, SLCO4A1, C9orf90, CSAG1, AMPH, MT1G, C20orf100, CST7, KIAA0367, RNASE1, HCP5, NDN, RAC2, AXL, BATF, EMILIN1, ARHGEF7, EVL, RAC2, KIAA0367, TFF3, CTSK, C21orf70, PRKCZ, MLSTD1, COL5A2, LY96, MAGEA1, MAGEC2, DENND2D, PPIE, SORBS2, PROCR, TMEPAI, SSNA1, and ZNF324 identifies 2 classes of stage 111C patients with significantly different survivals; and ii) signature F: FLJ25421, DENND2D, CTAG2, PGCP, KCNG1, MGMT, FAM107B, FANCE, GAGE2, Cllorfl, ABCB4, BGN, MAGEA10, SPATS1, CTNNAL1, PRRX1, EMP1, RNASE1, PDZD4, MAGEA6, CHMP4A, ADORA2B, HCP5, PLA1A, KIAA0141, OLFM1, ERO1L, CSAG1, SLCO4A1, CXorf12, C9orf90, CSAG1, AMPH, MT1G, ARHGDIB, C20orf100, CST7, KIAA0367, RNASE1, HCP5, NDN, RAC2, AXL, GRAMD3, ARSG, PLD1, HNMT, ZNF558, ATP6V1E2, EMILIN1, ARHGEF7, KRT25C, ZNF532, EVL, RAC2, SLAMF9, NBL1, KIAA0367, MAGEA10, TFF3, C21orf70, APOD, ABCA8, ACOX2, COL5A2, LY96, MAGEA1, MAGEC2, TIMP3, PPIE, RPUSD3, PROCR, and TMEPAI identifies 3 classes of stage IIIC patients with significantly different survivals. When, among the genes having significant (p<0.05) association between expression and methylation status, only those with a coefficient of correlation Rho<−0.4 were taken into account, an additional signature of 35 genes, signature G: PRDX2, DENND2D, CTAG2, PCOLCE, BGN, OCIAD2, RNASE1, MAGEA6, HCP5, OCIAD2, CSAG1, C9orf90, CSAG1, CTSK, ADM, RNASE1, HCP5, PAGES, NNMT, EMILIN1, MAGEA3, RAB11FIP5, ARHGDIB, EMILIN1, LPXN, CTSK, ALDH1A3, SUHW1, ABCA8, COL5A2, LY96, MAGEA1, MAGEC2, GSTT1, DENND2D was defined, which identifies 2 classes of stage IIIC patients with significantly different survivals. The identified multi-gene methylation signatures represent independent factors predicting prognosis of CM patients.

In addition to the prognostic value of the multi-gene methylation signatures above described, the methylation status of specific genes taken from these signatures may also have a prognostic impact per se, as exemplified in Example 4. Similarly, “methylation scores”, summarizing the above described genome-wide gene methylation profile of neoplastic cells, can also be utuilized to define prognosius of CM patients at the same stage of disease, see Example 5.

The extent of methylation of LINE-1 sequences and/or the methylation pattern of the (multi-)gene methylation signatures can be used, alone, or in conjunction with other biomarkers, to predict prognosis and response to therapy of CM patients, thus enabling the administration of patient-tailored clinical, follow-up and therapeutic procedures.

Accordingly, the invention provides a method of prognosing CM or of predicting and monitoring the efficacy of a CM therapy, in patients at the same clinico-pathological stage of disease, comprising measuring LINE-1 methylation and/or multi-gene methylation signatures in neoplastic cells. The term, “neoplastic cells” refers to any source of neoplastic cells from CM patients, including, but not limited to, primary cultures of autologous neoplastic cells, neoplastic cells purified from fresh autologous tumor tissues or from cryopreserved cell suspensions by immuno-sorting (e.g. using antibodies specific for cancer cells) or other means, cryopreserved tumor tissues, formalin fixed paraffin embedded (FFPE) tumor tissues, or other types of fixed tumor tissue, micro-/macro-dissected tumor tissues, circulating neoplastic cells present in body fluids (Tables 14-15, Example 7). Neoplastic cells may be obtained from a subject by any means known in the art.

As used herein, a “subject” is defined as a human. In a preferred embodiment the subject is a human with cancer. “Patient” refers to a human with cancer.

Methods for extracting genomic DNA are well-known in the art. Depending on the source of neoplastic cells, a preliminary step to remove embedding substances by organic solvents may be required. Classically, neoplastic cells from the subject are lysed using detergents and proteins are removed by different proteases. DNA is then purified from cell lysates either through extraction with organic solvents (e.g. phenol) or by affinity binding on silica-gel matrixes. DNA is then either precipitated with alcohols and dissolved in aqueous solutions, or washed with different solutions and eluted in aqueous solutions. Similar procedures are used to obtain genomic DNA from biologic fluids or circulating neoplastic cells.

The extent of methylation of LINE-1 is then measured on the DNA extracted from neoplastic cells. Different methods can be used to determine the extent of methylation of LINE-1, and are well-known in the art. These include, but are not limited to, Methylight, bisulfite sequencing, bisulfite pyrosequencing, Combined Bisulfite Restriction Analysis (CoBRA). Level of methylation of LINE-1 is typically defined as the ratio between the level of methylated LINE-1 and the level of methylated or unmethylated LINE-1, but any other mathematical formula relating to the level of LINE-1 methylation also applies. Furthermore, level of methylation of LINE-1 may refer to the extent of methylation at a single or multiple CpG sites as well as to aggregating measurements of methylation of multiple CpG sites, including, but not limited to, mean, median, mode. Similarly, the methylation status of all or any of the genes included in the 7 methylation signatures is measured on the DNA extracted from neoplastic cells. Different methods can be used to determine the extent of methylation of the multigene methylation signatures, and are well-known in the art. These include, but are not limited to, microarray-based methylation analysis, Methylight, bisulfite sequencing, bisulfite pyrosequencing, CoBRA. Level of methylation of each sequence/gene included in the signature is typically defined as the ratio between the level of methylated target sequence and the level of methylated or unmethylated target sequence, but any other mathematical formula relating to the level of gene/sequence methylation also applies. Furthermore, level of methylation of any gene/sequence included in the signature may refer to the extent of methylation at a single or multiple CpG sites as well as to aggregating measurements of methylation of multiple CpG sites, including, but not limited to, mean, median, mode.

MethyLight is the quantitative implementation of Methylation Specific PCR (MSP) using real-time PCR technology. MSP is a PCR-based technique that amplifies the target sequence depending on its methylation status. The approach requires an initial chemical modification of genomic DNA by incubation with sodium bisulfite, which converts unmethylated cytosines (C) to uracil (U), leaving methylated ones unaltered. This methylation-specific difference in the primary sequence generated by treatment is exploited to design oligonucleotide primers specific for either methylated or unmethylated target DNA, which are then employed in PCR-amplification reactions to discriminate between methylated and unmethylated sequences. Performing MSP reactions through real-time quantitative PCR approaches allows an efficient and sensitive quantification of the extent of methylation of the target sequence (MethyLight). Furthermore, when performing MethyLight using fluorogenic hybridization probe approaches (e.g., TaqMan® technology), the quantitative discrimination between methylated and unmethylated sequences may rely on selectivity for different methylation status of primers, probe or both.

Bisulfite sequencing allows to determine the methylation status of each CpG dinucleotide in the target sequence by conducting standard sequencing analysis. The technique relies on the differences in the sequence between methylated and unmethylated DNA that are generated by bisulfite treatment. The sequence of interest is PCR-amplified typically with primers amplifying all states, irrespective of methylation status. Usually, PCR products are cloned into plasmid vectors and single colonies are subjected to DNA sequencing through different methodologies available in the art. The extent of methylation at each CpG site is then typically established by evaluating the percentage of colonies with methylated CpG (i.e., C base in sense strand sequence).

Bisulfite pyrosequencing. Pyrosequencing is a high-throughput nonelectrophoretic nucleotide extensionsequencing technology for various applications includingDNA methylation analysis. Pyrosequencing-based methylation analysis investigates quantitatively the degree of methylation at CpG positions after treatment of genomic DNA with sodium bisulfite. The target region is amplified by PCR using a pair of primers complementary to the bisulfite-treated DNA sequence, amplifying all states, irrespective of methylation status. Usually, one of these two amplification primers carries a biotin label at the 5′-end that is exploited to subsequently purify the PCR product by immobilization on streptavidin-coated sepharose beads. Immobilized PCR product is then rendered single-stranded by alkali and hybridized to a pyrosequencing primer complementary to a region in close proximity to the sequence for which methylation data is going to be analyzed. The pyrosequencing reaction is performed by the sequential addition of single nucleotides in a predefined order. Each incorporation event is accompanied by release of pyrophosphate (PPi) in a quantity equimolar to the amount of nucleotide incorporated. ATP sulfurylase quantitatively converts PPi to ATP in the presence of adenosine 5′ phosphosulfate. ATP then drives the luciferase-mediated conversion of luciferin to oxyluciferin that generates visible light in amounts that are proportional to the amount of ATP. Light emission is detected by an instrument and reported as a peak which area represents the quantity of the respective nucleotide. The level of methylation at each CpG position in a sequence is usually determined from the ratio of C over the sum of C and T.

CoBRA is a technique that enables quantitative evaluation of DNA methylation by exploiting the sequence differences that exist between methylated and unmethylated DNA upon treatment with sodium bisulfite. In particular, it relies on the generation, or suppression, of a recognition site for restriction enzymes depending on the methylation status of the sequence under analysis. The target region is amplified by PCR using a pair of primers complementary to the bisulfite-treated DNA sequence, amplifying all states, irrespective of methylation status. After amplification, PCR products are digested with a specific restriction enzyme that cuts only DNA molecules that were methylated (or unmethyated) before bisulfite treatment. Digestion products are then separated by conventional, microfluidic or capillary electrophoresis, and the proportion of digested versus undigested products is quantitated and used to determine the extent of methylation at that particular site.

High-throughput DNA microarray technologies can be efficiently used to define genome-wide gene methylation profiles. Different methodologies either rely on the enrichment of genomic DNA for methylated sequences, through methylated DNA immunoprecipitation (MeDIP) approaches, or on the modification of the genomic DNA by sodium bisulfite. MeDIP can be conducted utilizing either anti-5-methylcytosine antibodies or proteins which selectively bind to methylated DNA (e.g., methyl-CpG-binding domain (MBD) proteins). Capturing of the genomic DNA with these reagents bound to an insoluble matrix allows to enrich the sample for methylated sequences. Classically, for each test sample, genomic DNA immunoprecipitated (IP) and DNA non-IP, utilized as control, are labelled with different fluorescently-coupled nucleotides (e.g., Cy5-dCTP for IP DNA and Cy3-dCTP for non-IP DNA), and co-hybridized to a microarray containing the probes for the sequence set of interest (e.g., CpG island arrays representing over 27,000 CpG sites distributed genome-wide). Fluorescence intensity data are then extracted from the scanned microarrays, pre-processed and analyzed using different software packages to provide a methylation score for each analyzed CpG site. On the other hand, the treatment of genomic DNA with sodium bisulfite translates the methylation data into primary DNA sequence modifications that are interrogated using oligonucleotide probes. Typically, bisulfate-modified genomic DNA is evaluated at each locus through hybridization of a pair of oligonucleotide probes constituted by one probe specific for the methylated and one specific for the unmethylated target sequence. Probes can be elongated by a polymerase only in case of match, so that probes for methylated or unmethylated sequences are quantitatively elongated only in the presence of methylated or unmethylated sequences in the test genomic DNA, respectively. A classic application employs single-base probe extension using fluorescently-labelled nucleotides to reveal the methylation status at each locus. The level of methylation for the interrogated locus can be determined by calculating the ratio of the fluorescent signals from the methylated vs unmethylated probes. In a typical application, this technology enables to interrogate the genome-wide methylation status of over 27,000 CpG sites covering over 14,000 genes. Besides microarray technology, the identity of methylated sequences can also be determined by applying next generation sequencing approaches to the MeDIP-enriched or the bisulfite-modified genomic DNA. The identification of methylation profiles (multi-gene methylation signatures) specifically associated with different prognosis (e.g., OS, time to progression (TTP), CM-specific survival) of patients may be obtained through different approaches including, but not limited to, that illustrated in Example 3.

The invention further provides a method for determining prognosis (including, but not limited to, OS, disease- and progression-free survival, CM-specific survival) of subjects with CM within a clinico-pathological stage of disease. In one such method, a neoplastic cell sample is obtained from a subject and the level of LINE-1 methylation is determined. If the level of LINE-1 methylation in the sample is lower than a reference value, the subject survives longer. On the contrary, if the level of LINE-1 methylation in the sample is higher than a reference value, the subject survives less. The reference value of LINE-1 methylation is determined in a test population of subjects with cancer as the value that more specifically and sensitively discriminates patients with different survivals. This reference value can be defined through any approach available in the art, including, but not limited to, receiver-operator characteristic (ROC) curve analysis, mean, median, mode value of LINE-1 methylation of neoplastic cells from the population of cancer patients under evaluation. In an additional method for determining prognosis, a neoplastic cell sample is obtained from a subject affected by CM, and the methylation profile of any, multiple or all of the genes included in the multi-gene methylation signatures (Table 3) is evaluated. The methylation profile obtained from the neoplastic cells of the patient is then fed into a methylation-based classifier algorithm that will assign patients at the same stage of disease into either a high- or a low-risk group. Patients assigned to the high-risk group based on their (multi-)gene methylation profile will survive less, while patients assigned to the low-risk group will survive longer. Furthermore, combining both LINE-1 methylation and gene methylation signature classifications of patients results in a stronger prognostic marker, which, for instance, predicts that patients whose neoplastic cells have LINE-1 methylation lower than a reference value and a low-risk methylation signature have a longer survival (Example 6).

In another method of determining prognosis, a biological fluid sample is obtained from a subject and is evaluated for LINE-1 methylation and/or for (multi-)gene methylation signatures. A “biological fluid” refers to any fluid present in the subject in which acellular DNA or cancer cells may be present, including, without limitation, blood, serum, plasma, bone marrow, cerebrospinal fluid, peritoneal/pleural fluid, lymph fluid, ascitic, serous fluid, sputum, lacrimal fluid, stool, and urine. Body fluid samples can be obtained from a subject using any of the methods known in the art.

Accordingly to the above reported role of LINE-1 methylation level and of (multi-) gene methylation signatures as prognostic markers, the invention includes the measurement and use of LINE-1 methylation and/or of (multi-)gene methylation signatures to define differentiated therapeutic treatments and differentiated follow-up procedures and schedules for CM patients, according to their different prognosis as defined by LINE-1 methylation and/or (multi-)gene methylation signatures.

The invention further provides LINE-1 methylation level as a surrogate marker of presence and levels of expression in neoplastic cells of different therapeutic targets regulated by DNA methylation, including, but not limited to, Cancer Testis Antigens of the MAGE, GAGE, NY-ESO, SSX families, High Molecular Weight-Melanoma Associated Antigen, class I and II Human Leukocyte Antigens, and Intercellular Adhesion Molecule-1. In such a method, a neoplastic cell sample is obtained from a subject/patient and the level of LINE-1 methylation is determined. If the level of LINE-1 methylation in the sample is lower than a reference value, neoplastic cells are likely to express a higher number of distinct methylation-regulated therapeutic targets and/or increased levels of their expression. As a consequence, this population of patients is most likely to respond to any therapeutic treatment (including, but not limited to, chemotherapy, immunotherapy, biologic therapy, radiotherapy and targeted therapy utilized alone or in combination) involving methylation-regulated molecules, and thus it represents the preferred cohort of individuals to be treated with these therapies utilizing a patient-tailored approach. Accordingly, the invention includes the use of the evaluation of the extent of LINE-1 methylation to identify patients to be treated with therapeutic approaches targeting one or multiple DNA methylation-regulated molecules, including, but not limited to, Cancer Testis Antigens of the MAGE, GAGE, NY-ESO, SSX families, High Molecular Weight-Melanoma Associated Antigen, class I and II Human Leukocyte Antigens, and Intercellular Adhesion Molecule-1.

The invention also provides a method for predicting response to therapy. In such a method, a neoplastic cell sample is obtained from a subject/patient and the level of LINE-1 methylation and/or the (multi-)gene methylation signatures are determined. The level of LINE-1 methylation and/or the (multi-)gene methylation signature is associated with the fact that the patient is more likely to respond better to different therapeutic approaches, including, but not limited to, immunotherapy, chemotherapy, radiotherapy, biologic therapy, targeted therapy utilized alone or in combination. Accordingly, the invention includes the use of the level of LINE-1 methylation and or of (multi-)gene methylation signatures for selecting patients to be treated with specific therapeutic approaches.

Patients whose neoplastic cells have a level of LINE-1 methylation lower than a reference value have a more favorable prognosis, surviving longer. Similarly, patients whose neoplastic cells have more hypomethylated multi-gene methylation signatures (see Examples 3 and 5) are characterized by a longer survival. Accordingly, one aspect of the invention includes the administration of compounds able to reduce the extent of methylation of genomic DNA (DNA hypomethylating agents, DHA) in cancer patients whose neoplastic cells have levels of LINE-1 methylation higher than a reference value and/or hypermethylated (multi-) gene methylation signatures. The hypomethylation of genomic DNA, of LINE-1 DNA, and of the genes included in the methylation signatures in neoplastic cells of these treated patients is expected to drive cells to a less aggressive phenotype and to provide patients with a longer survival. Treatment of patients may be done with DHA alone or in combination with any other therapeutic option available. In one, not limiting, embodiment, pharmacologic DNA hypomethylation could be used as a feasible chemoprevention approach in the initial phases of disease and/or in patients at high-risk of disease recurrence.

It is therefore an object of the present invention an in vitro method of prognosing melanoma within a clinico-pathological stage thereof and/or monitoring the response to a melanoma therapy in a subject and/or to identify a subject to be treated with a DNA hypomethylating agent comprising determining the methylation status of at least one cytosine residue in a methylation relevant region of at least 16 genes selected from the group of: ABCA8, ABCB4, ACOX2, ACSL5, ADM, ADORA2B, ALDH1A3, ALOX12B, AMPH, ANGPT4, ANGPTL3, ANKRD1, ANXA13, ANXA8, APOBEC4, APOD, ARHGDIB, ARHGEF7, ARSG, ATP6V1E2, AVPR1B, AXL, BATF, BGN, BSND, Cllorfl, Clorf201, Clorf36, Clorf42, Clorf64, C20orf100, C21orf70, C3orf27, C3orf63, C9orf90, CD1D, CEACAM6, CHMP4A, CLEC4F, COL5A2, CPA3, CRHR1, CSAG1, CST11, CST7, CST9L, CTAG1B, CTAG2, CTNNAL1, CTSK, CX3CR1, CXorf12, CYP2A7, CYP2B6, CYP8B1, DARC, DCLRE1A, DEFB123, DEFB4, DENND2D, DIRC1, DLG5, EMILIN1, EMP1, ERO1L, EVL, FAAH, FAM107B, FAM38B, FANCE, FGF4, FLJ20581, FLJ25421, FLJ32771, FLJ33860, FLJ39501, FMO2, FOXF2, FOXI1, FOXN1, FXYD4, GABRG3, GAGE2, GAS2L2, GJB3, GJB4, GJB5, GLT1D1, GRAMD3, GRM4, GSTT1, GUCA2B, HAO2, HCP5, HLA-DOB, HNMT, IFT140, IGLL1, IL17F, IL28B, IL8RA, IL8RB, KCNG1, KCNK16, KIAA0141, KIAA0367, KRT25C, KRT25D, KRTHB2, KRTHB3, KRTHB4, KRTHB5, LAMC2, LOC124751, LPXN, LRRN5, LY96, MAGEA1, MAGEA10, MAGEA3, MAGEA6, MAGEC2, MGC29671, MGC35206, MGMT, MIA2, MIS12, MLN, MLSTD1, MRVI1, MSMB, MT1G, NALP10, NBL1, NDN, NNMT, NR5A2, OCIAD2, OLFM1, OR12D3, PAGES, PALLD, PCDHAC2, PCOLCE, PDZD4, PGCP, PKHD1, PLA1A, PLD1, PPAT, PPIE, PRDX2, PREPL, PRKCZ, PROCR, PRRX1, PSD4, PTCD2, PVALB, R3HDML, RAB11FIP5, RAC2, RNASE1, RPUSD3, S100A9, SCN4A, SEMG2, SERPINB12, SFTPG, SLAMF9, SLC13A1, SLC18A2, SLC22A8, SLC2A7, SLC44A5, SLC4A7, SLC6A11, SLC6A18, SLCO4A1, SLPI, SORBS2, SPATS1, SPINKS, SSNA1, STK38, SUHW1, TDRDS, TFF3, THRB, TIMP3, TMEM10, TMEPAI, TNFSF13B, TNNI1, TRIM40, TUB, UBE2E1, UNC45B, UNQ9433, WDR20, WFDC12, WFDC8, WNT10B, ZNF324, ZNF532, ZNF558, or a variant thereof, in a biological sample obtained from the subject.

Preferably, the genes are: ALOX12B, CRHR1, FGF4, FLJ33860, GJB5, GRM4, IGLL1, MGC35206, PCDHAC2, S100A9, SLC18A2, SLC6A11, SLC6A18, TRIM40, TUB and WNT10B (signature C) and allelic variants thereof.

Still preferably, for each of said genes, the methylation relevant regions are comprised in the nucleotide sequences as indicated in Table 3 signature C, or in complementary sequences thereof.

Yet preferably, for each of said genes, the methylation relevant regions consist of: nt 940 to nt 1061 of SEQ ID No. 2, nt 941 to nt 1062 of SEQ ID No. 4, nt 940 to nt 1061 of SEQ ID No. 7, nt 941 to nt 1062 of SEQ ID No. 10, nt 940 to nt 1061 of SEQ ID No. 11, nt 940 to nt 1061 of SEQ ID No. 16, nt 940 to nt 1061 of SEQ ID No. 17, nt 941 to nt 1062 of SEQ ID No. 86, nt 941 to nt 1062 of SEQ ID No. 99, nt 940 to nt 1061 of SEQ ID No. 100, nt 940 to nt 1061 of SEQ ID No. 101, nt 941 to nt 1062 of SEQ ID No. 102, nt 941 to nt 1062 of SEQ ID No. 103, nt 940 to nt 1061 of SEQ ID No. 104, nt 941 to nt 1062 of SEQ ID No. 105, nt 940 to nt 1061 of SEQ ID No. 106, nt 940 to nt 1061 of SEQ ID No. 107, an allelic variant or a complementary sequence thereof.

In a preferred embodiment the genes are: ANXA8, CRHR1, CST11, CYP2B6, CYP8B1, FLJ33860, FOXI1, FOXN1, FXYD4, GAS2L2, GJB5, GUCA2B, IGLL1, IL8RA, KCNK16, KRTHB2, MGC29671, OR12D3, S100A9, SLPI, TMEM10, TRIM40, TUB, and WFDC8 (signature A) and allelic variants thereof.

Preferably, for each of said genes, the methylation relevant regions are comprised in the nucleotide sequences as indicated in Table 3 signature A, or in complementary sequences thereof.

Still preferably for each of said genes, the methylation relevant regions consist of: nt 941 to nt 1062 of SEQ ID No. 1, nt 940 to nt 1061 of SEQ ID No. 2, nt 940 to nt 1061 of SEQ ID No. 3, nt 941 to nt 1062 of SEQ ID No. 4, nt 940 to nt 1061 of SEQ ID No. 5, nt 940 to nt 1061 of SEQ ID No. 6, nt 940 to nt 1061 of SEQ ID No. 7, nt 941 to nt 1062 of SEQ ID No. 8, nt 941 to nt 1062 of SEQ ID No. 9, nt 941 to nt 1062 of SEQ ID No. 10, nt 940 to nt 1061 of SEQ ID No. 11, nt 940 to nt 1061 of SEQ ID No. 12, nt 941 to nt 1062 of SEQ ID No. 13, nt 940 to nt 1061 of SEQ ID No. 14, nt 941 to nt 1062 of SEQ ID No. 15, nt 940 to nt 1061 of SEQ ID No. 16, nt 940 to nt 1061 of SEQ ID No. 17, nt 941 to nt 1062 of SEQ ID No. 18, nt 940 to nt 1061 of SEQ ID No. 19, nt 940 to nt 1061 of SEQ ID No. 20, nt 940 to nt 1061 of SEQ ID No. 21, nt 940 to nt 1061 of SEQ ID No. 22, nt 940 to nt 1061 of SEQ ID No. 23, nt 940 to nt 1061 of SEQ ID No. 24, an allelic variant or a complementary sequence thereof.

In a preferred embodiment the genes are: ABCA8, ADM, ALDH1A3, ARHGDIB, BGN, C9orf90, COL5A2, CSAG1, CTAG2, CTSK, DENND2D, EMILIN1, GSTT1, HCP5, LPXN, LY96, MAGEA1, MAGEA3, MAGEA6, MAGEC2, NNMT, OCIAD2, PAGES, PCOLCE, PRDX2, RAB11FIP5, RNASE1 and SUHW1 (signature G) and allelic variants thereof. Preferably for each of said genes, the methylation relevant regions are comprised in the nucleotide sequences as indicated in Table 3 signature G, or in complementary sequences thereof.

Still preferably for each of said genes, the methylation relevant regions consist of: nt 940 to nt 1061 of SEQ ID No. 109, nt 940 to nt 1061 of SEQ ID No. 125, nt 940 to nt 1061 of SEQ ID No. 132, nt 940 to nt 1061 of SEQ ID No. 138, nt 941 to nt 1062 of SEQ ID No. 139, nt 940 to nt 1061 of SEQ ID No. 140, nt 941 to nt 1062 of SEQ ID No. 142, nt 941 to nt 1062 of SEQ ID No. 143, nt 941 to nt 1062 of SEQ ID No. 149, nt 941 to nt 1062 of SEQ ID No. 150, nt 941 to nt 1062 of SEQ ID No. 155, nt 941 to nt 1062 of SEQ ID No. 161, nt 941 to nt 1062 of SEQ ID No. 165, nt 941 to nt 1062 of SEQ ID No. 166, nt 941 to nt 1062 of SEQ ID No. 167, nt 941 to nt 1062 of SEQ ID No. 168, nt 941 to nt 1062 of SEQ ID No. 169, nt 941 to nt 1062 of SEQ ID No. 181, nt 941 to nt 1062 of SEQ ID No. 182, nt 940 to nt 1061 of SEQ ID No. 201, nt 940 to nt 1061 of SEQ ID No. 205, nt 940 to nt 1061 of SEQ ID No. 206, nt 941 to nt 1062 of SEQ ID No. 207, nt 940 to nt 1061 of SEQ ID No. 208, nt 940 to nt 1061 of SEQ ID No. 209, nt 940 to nt 1061 of SEQ ID No. 210, nt 941 to nt 1062 of SEQ ID No. 211, nt 941 to nt 1062 of SEQ ID No. 212, nt 941 to nt 1062 of SEQ ID No. 213, nt 940 to nt 1061 of SEQ ID No. 214, nt 940 to nt 1061 of SEQ ID No. 215, nt 941 to nt 1062 of SEQ ID No. 216, nt 941 to nt 1062 of SEQ ID No. 217, nt 940 to nt 1061 of SEQ ID No. 218, nt 940 to nt 1061 of SEQ ID No. 219, an allelic variant or a complementary sequence thereof.

In a preferred embodiment the genes are: ACSL5, ALOX12B, ANKRD1, ANXA13, APOBEC4, AVPR1B, Clorf201, CD1D, CEACAM6, CRHR1, CTAG1B, CTAG2, DCLRE1A, DIRC1, FAAH, FGF4, FLJ25421, FLJ33860, FLJ39501, GABRG3, GJB3, GJB5, GLT1D1, GRM4, HAO2, IFT140, IL28B, KCNK16, LRRN5, MGC35206, MIA2, OR12D3, PCDHAC2, PVALB, S100A9, SLC18A2, SLC4A7, SLC6A11, SLC6A18, STK38, TDRD5, TRIM40, TUB, UBE2E1, UNQ9433 and WNT10B (signature D) or allelic variant thereof.

Preferably for each of said genes, the methylation relevant regions are comprised in the nucleotide sequences as indicated in Table 3 signature D, or in complementary sequences thereof.

Still preferably for each of said genes, the methylation relevant region consist of: nt 940 to nt 1061 of SEQ ID No. 2, nt 940 to nt 1061 of SEQ ID No. 3, nt 940 to nt 1061 of SEQ ID No. 6, nt 940 to nt 1061 of SEQ ID No. 7, nt 941 to nt 1062 of SEQ ID No. 10, nt 940 to nt 1061 of SEQ ID No. 11, nt 940 to nt 1061 of SEQ ID No. 16, nt 940 to nt 1061 of SEQ ID No. 17, nt 940 to nt 1061 of SEQ ID No. 25, nt 940 to nt 1061 of SEQ ID No. 30, nt 940 to nt 1061 of SEQ ID No. 34, nt 940 to nt 1061 of SEQ ID No. 38, nt 940 to nt 1061 of SEQ ID No. 39, nt 940 to nt 1061 of SEQ ID No. 42, nt 940 to nt 1061 of SEQ ID No. 46, nt 940 to nt 1061 of SEQ ID No. 48, nt 940 to nt 1061 of SEQ ID No. 56, nt 940 to nt 1061 of SEQ ID No. 59, nt 940 to nt 1061 of SEQ ID No. 63, nt 941 to nt 1062 of SEQ ID No. 86, nt 940 to nt 1061 of SEQ ID No. 94, nt 941 to nt 1062 of SEQ ID No. 99, nt 940 to nt 1061 of SEQ ID No. 100, nt 940 to nt 1061 of SEQ ID No. 101, nt 941 to nt 1062 of SEQ ID No. 102, nt 941 to nt 1062 of SEQ ID No. 103, nt 940 to nt 1061 of SEQ ID No. 104, nt 941 to nt 1062 of SEQ ID No. 105, nt 940 to nt 1061 of SEQ ID No. 106, nt 940 to nt 1061 of SEQ ID No. 107, nt 940 to nt 1061 of SEQ ID No. 108, nt 940 to nt 1061 of SEQ ID No. 109, nt 940 to nt 1061 of SEQ ID No. 110, nt 940 to nt 1061 of SEQ ID No. 111, nt 940 to nt 1061 of SEQ ID No. 112, nt 941 to nt 1062 of SEQ ID No. 113, nt 941 to nt 1062 of SEQ ID No. 114, nt 941 to nt 1062 of SEQ ID No. 115, nt 940 to nt 1061 of SEQ ID No. 116, nt 941 to nt 1062 of SEQ ID No. 117, nt 941 to nt 1062 of SEQ ID No. 118, nt 940 to nt 1061 of SEQ ID No. 119, nt 941 to nt 1062 of SEQ ID No. 120, nt 940 to nt 1061 of SEQ ID No. 121, nt 941 to nt 1062 of SEQ ID No. 122, nt 941 to nt 1062 of SEQ ID No. 123, nt 940 to nt 1061 of SEQ ID No. 124, an allelic variant or a complementary sequence thereof.

In a preferred embodiment the genes are: AMPH, ARHGEF7, AXL, BATF, BGN, C20orf100, C21orf70, C9orf90, COL5A2, CSAG1, CST7, CTAG2, CTSK, DENND2D, EMILIN1, EVL, FAM107B, FAM38B, FANCE, FOXF2, HCP5, KCNG1, KIAA0367, LY96, MAGEA1, MAGEA10, MAGEC2, MLSTD1, MT1G, NDN, OCIAD2, PDZD4, PGCP, PPIE, PRKCZ, PROCR, PRRX1, RAC2, RNASE1, SLCO4A1, SORBS2, SPATS1, SSNA1, TFF3, TMEPAI and ZNF324 (signature E) or allelic variants thereof.

Preferably for each of said genes, the methylation relevant regions are comprised in the nucleotide sequences as indicated in Table 3 signature E, or in complementary sequences thereof.

Still preferably for each of said genes, the methylation relevant region consist of: nt 940 to nt 1061 of SEQ ID No. 109, nt 940 to nt 1061 of SEQ ID No. 125, nt 940 to nt 1061 of SEQ ID No. 126, nt 941 to nt 1062 of SEQ ID No. 127, nt 941 to nt 1062 of SEQ ID No. 128, nt 941 to nt 1062 of SEQ ID No. 129, nt 941 to nt 1062 of SEQ ID No. 130, nt 941 to nt 1062 of SEQ ID No. 131, nt 940 to nt 1061 of SEQ ID No. 132, nt 940 to nt 1061 of SEQ ID No. 133, nt 941 to nt 1062 of SEQ ID No. 134, nt 940 to nt 1061 of SEQ ID No. 135, nt 941 to nt 1062 of SEQ ID No. 136, nt 941 to nt 1062 of SEQ ID No. 137, nt 940 to nt 1061 of SEQ ID No. 138, nt 941 to nt 1062 of SEQ ID No. 139, nt 940 to nt 1061 of SEQ ID No. 140, nt 940 to nt 1061 of SEQ ID No. 141, nt 941 to nt 1062 of SEQ ID No. 142, nt 941 to nt 1062 of SEQ ID No. 143, nt 941 to nt 1062 of SEQ ID No. 144, nt 941 to nt 1062 of SEQ ID No. 145, nt 941 to nt 1062 of SEQ ID No. 146, nt 941 to nt 1062 of SEQ ID No. 147, nt 941 to nt 1062 of SEQ ID No. 148, nt 941 to nt 1062 of SEQ ID No. 149, nt 941 to nt 1062 of SEQ ID No. 150, nt 940 to nt 1061 of SEQ ID No. 151, nt 940 to nt 1061 of SEQ ID No. 152, nt 940 to nt 1061 of SEQ ID No. 153, nt 941 to nt 1062 of SEQ ID No. 154, nt 941 to nt 1062 of SEQ ID No. 155, nt 940 to nt 1061 of SEQ ID No. 156, nt 940 to nt 1061 of SEQ ID No. 157, nt 940 to nt 1061 of SEQ ID No. 158, nt 941 to nt 1062 of SEQ ID No. 159, nt 940 to nt 1061 of SEQ ID No. 160, nt 941 to nt 1062 of SEQ ID No. 161, nt 940 to nt 1061 of SEQ ID No. 162, nt 940 to nt 1061 of SEQ ID No. 163, nt 940 to nt 1061 of SEQ ID No. 164, nt 941 to nt 1062 of SEQ ID No. 165, nt 941 to nt 1062 of SEQ ID No. 166, nt 941 to nt 1062 of SEQ ID No. 167, nt 941 to nt 1062 of SEQ ID No. 168, nt 941 to nt 1062 of SEQ ID No. 169, nt 940 to nt 1061 of SEQ ID No. 170, nt 940 to nt 1061 of SEQ ID No. 171, nt 941 to nt 1062 of SEQ ID No. 172, nt 940 to nt 1061 of SEQ ID No. 173, nt 941 to nt 1062 of SEQ ID No. 174, nt 941 to nt 1062 of SEQ ID No. 175, an allelic variant or a complementary sequence thereof.

In a preferred embodiment the genes are: ABCA8, ABCB4, ACOX2, ADORA2B, AMPH, APOD, ARHGDIB, ARHGEF7, ARSG, ATP6V1E2, AXL, BGN, Cllorfl, C20orf100, C21orf70, C9orf90, CHMP4A, COL5A2, CSAG1, CST7, CTAG2, CTNNAL1, CXorf12, DENND2D, EMILIN1, EMP1, ERO1L, EVL, FAM107B, FANCE, FLJ25421, GAGE2, GRAMD3, HCP5, HNMT, KCNG1, KIAA0141, KIAA0367, KRT25C, LY96, MAGEA1, MAGEA10, MAGEA6, MAGEC2, MGMT, MT1G, NBL1, NDN, OLFM1, PDZD4, PGCP, PLA1A, PLD1, PPIE, PROCR, PRRX1, RAC2, RNASE1, RPUSD3, SLAMF9, SLCO4A1, SPATS1, TFF3, TIMP3, TMEPAI, ZNF532 and ZNF558 (signature F) or alleleic variants thereof.

Preferably for each of said genes, the methylation relevant regions are comprised in the nucleotide sequences as indicated in Table 3 signature F, or in complementary sequences thereof.

Still preferably for each of said genes, the methylation relevant region consist of: nt 940 to nt 1061 of SEQ ID No. 25, nt 941 to nt 1062 of SEQ ID No. 61, nt 941 to nt 1062 of SEQ ID No. 80, nt 940 to nt 1061 of SEQ ID No. 109, nt 940 to nt 1061 of SEQ ID No. 125, nt 940 to nt 1061 of SEQ ID No. 126, nt 941 to nt 1062 of SEQ ID No. 127, nt 941 to nt 1062 of SEQ ID No. 128, nt 941 to nt 1062 of SEQ ID No. 129, nt 940 to nt 1061 of SEQ ID No. 132, nt 940 to nt 1061 of SEQ ID No. 133, nt 941 to nt 1062 of SEQ ID No. 134, nt 940 to nt 1061 of SEQ ID No. 135, nt 941 to nt 1062 of SEQ ID No. 137, nt 940 to nt 1061 of SEQ ID No. 138, nt 940 to nt 1061 of SEQ ID No. 140, nt 940 to nt 1061 of SEQ ID No. 141, nt 941 to nt 1062 of SEQ ID No. 142, nt 941 to nt 1062 of SEQ ID No. 143, nt 941 to nt 1062 of SEQ ID No. 144, nt 941 to nt 1062 of SEQ ID No. 145, nt 941 to nt 1062 of SEQ ID No. 146, nt 941 to nt 1062 of SEQ ID No. 147, nt 941 to nt 1062 of SEQ ID No. 148, nt 941 to nt 1062 of SEQ ID No. 149, nt 941 to nt 1062 of SEQ ID No. 150, nt 940 to nt 1061 of SEQ ID No. 151, nt 940 to nt 1061 of SEQ ID No. 152, nt 940 to nt 1061 of SEQ ID No. 153, nt 941 to nt 1062 of SEQ ID No. 155, nt 940 to nt 1061 of SEQ ID No. 156, nt 940 to nt 1061 of SEQ ID No. 157, nt 940 to nt 1061 of SEQ ID No. 158, nt 941 to nt 1062 of SEQ ID No. 159, nt 940 to nt 1061 of SEQ ID No. 160, nt 940 to nt 1061 of SEQ ID No. 162, nt 941 to nt 1062 of SEQ ID No. 165, nt 941 to nt 1062 of SEQ ID No. 166, nt 941 to nt 1062 of SEQ ID No. 167, nt 941 to nt 1062 of SEQ ID No. 168, nt 940 to nt 1061 of SEQ ID No. 170, nt 941 to nt 1062 of SEQ ID No. 172, nt 940 to nt 1061 of SEQ ID No. 173, nt 941 to nt 1062 of SEQ ID No. 175, nt 941 to nt 1062 of SEQ ID No. 176, nt 941 to nt 1062 of SEQ ID No. 177, nt 940 to nt 1061 of SEQ ID No. 178, nt 941 to nt 1062 of SEQ ID No. 179, nt 941 to nt 1062 of SEQ ID No. 180, nt 941 to nt 1062 of SEQ ID No. 181, nt 941 to nt 1062 of SEQ ID No. 182, nt 940 to nt 1061 of SEQ ID No. 183, nt 940 to nt 1061 of SEQ ID No. 184, nt 940 to nt 1061 of SEQ ID No. 185, nt 940 to nt 1061 of SEQ ID No. 186, nt 940 to nt 1061 of SEQ ID No. 187, nt 940 to nt 1061 of SEQ ID No. 188, nt 940 to nt 1061 of SEQ ID No. 189, nt 940 to nt 1061 of SEQ ID No. 190, nt 941 to nt 1062 of SEQ ID No. 191, nt 940 to nt 1061 of SEQ ID No. 192, nt 940 to nt 1061 of SEQ ID No. 193, nt 941 to nt 1062 of SEQ ID No. 194, nt 940 to nt 1061 of SEQ ID No. 195, nt 940 to nt 1061 of SEQ ID No. 196, nt 941 to nt 1062 of SEQ ID No. 197, nt 941 to nt 1062 of SEQ ID No. 198, nt 941 to nt 1062 of SEQ ID No. 199, nt 940 to nt 1061 of SEQ ID No. 200, nt 940 to nt 1061 of SEQ ID No. 201, nt 940 to nt 1061 of SEQ ID No. 202, nt 940 to nt 1061 of SEQ ID No. 203, nt 940 to nt 1061 of SEQ ID No. 204, an allelic variant or a complementary sequence thereof.

In a preferred embodiment the genes are: ACSL5, ANGPT4, ANGPTL3, ANKRD1, ANXA8, APOBEC4, APOD, ARS G, B SND, Clorf201, Clorf36, Clorf42, Clorf64, C3 orf27, C3 orf63, CLEC4F, CPA3, CRHR1, CST11, CST9L, CX3CR1, CYP2A7, CYP2B6, CYP8B1, DARC, DCLRE1A, DEFB123, DEFB4, DIRC1, DLG5, F1120581, FLJ25421, FLJ32771, FLJ3860, FMO2, FOXI1, FOXN1, FXYD4, GAS2L2, GJB4, GJB5, GUCA2B, HLA-DOB, IFT140, IGLL1, IL17F, IL8RA, IL8RB, KCNK16, KRT25D, KRTHB2, KRTHB3, KRTHB4, KRTHB5, LAMC2, LOC124751, LRRN5, MGC29671, MGC35206, MIA2, MIS12, MLN, MRVI1, MSMB, NALP10, NR5A2, OR12D3, PALLD, PKHD1, PPAT, PREPL, PSD4, PTCD2, R3HDML, S100A9, SCN4A, SEMG2, SERPINB12, SFTPG, SLC13A1, SLC22A8, SLC2A7, SLC44A5, SLPI, SPINK5, STK38, THRB, TMEM10, TNFSF13B, TNNI1, TRIM40, TUB, UBE2E1, UNC45B, WDR20, WFDC12 and WFDC8 (signature B) or allelic variants thereof.

Preferably for each of said genes, the methylation relevant regions are comprised in the nucleotide sequences as indicated in Table 3 signature B, or in complementary sequences thereof.

Still preferably for each of said genes, the methylation relevant regions consist of: nt 941 to nt 1062 of SEQ ID No. 1, nt 940 to nt 1061 of SEQ ID No. 2, nt 940 to nt 1061 of SEQ ID No. 3, nt 941 to nt 1062 of SEQ ID No. 4, nt 940 to nt 1061 of SEQ ID No. 5, nt 940 to nt 1061 of SEQ ID No. 6, nt 940 to nt 1061 of SEQ ID No. 7, nt 941 to nt 1062 of SEQ ID No. 8, nt 941 to nt 1062 of SEQ ID No. 9, nt 941 to nt 1062 of SEQ ID No. 10, nt 940 to nt 1061 of SEQ ID No. 11, nt 940 to nt 1061 of SEQ ID No. 12, nt 941 to nt 1062 of SEQ ID No. 13, nt 940 to nt 1061 of SEQ ID No. 14, nt 941 to nt 1062 of SEQ ID No. 15, nt 940 to nt 1061 of SEQ ID No. 16, nt 940 to nt 1061 of SEQ ID No. 17, nt 941 to nt 1062 of SEQ ID No. 18, nt 940 to nt 1061 of SEQ ID No. 19, nt 940 to nt 1061 of SEQ ID No. 20, nt 940 to nt 1061 of SEQ ID No. 21, nt 940 to nt 1061 of SEQ ID No. 22, nt 940 to nt 1061 of SEQ ID No. 23, nt 940 to nt 1061 of SEQ ID No. 24, nt 940 to nt 1061 of SEQ ID No. 25, nt 941 to nt 1062 of SEQ ID No. 26, nt 941 to nt 1062 of SEQ ID No. 27, nt 941 to nt 1062 of SEQ ID No. 28, nt 940 to nt 1061 of SEQ ID No. 29, nt 940 to nt 1061 of SEQ ID No. 30, nt 940 to nt 1061 of SEQ ID No. 31, nt 940 to nt 1061 of SEQ ID No. 32, nt 940 to nt 1061 of SEQ ID No. 33, nt 940 to nt 1061 of SEQ ID No. 34, nt 940 to nt 1061 of SEQ ID No. 35, nt 940 to nt 1061 of SEQ ID No. 36, nt 940 to nt 1061 of SEQ ID No. 37, nt 940 to nt 1061 of SEQ ID No. 38, nt 940 to nt 1061 of SEQ ID No. 39, nt 941 to nt 1062 of SEQ ID No. 40, nt 940 to nt 1061 of SEQ ID No. 41, nt 940 to nt 1061 of SEQ ID No. 42, nt 940 to nt 1061 of SEQ ID No. 43, nt 940 to nt 1061 of SEQ ID No. 44, nt 941 to nt 1062 of SEQ ID No. 45, nt 940 to nt 1061 of SEQ ID No. 46, nt 941 to nt 1062 of SEQ ID No. 47, nt 940 to nt 1061 of SEQ ID No. 48, nt 941 to nt 1062 of SEQ ID No. 49, nt 940 to nt 1061 of SEQ ID No. 50, nt 940 to nt 1061 of SEQ ID No. 51, nt 940 to nt 1061 of SEQ ID No. 52, nt 941 to nt 1062 of SEQ ID No. 53, nt 941 to nt 1062 of SEQ ID No. 54, nt 940 to nt 1061 of SEQ ID No. 55, nt 940 to nt 1061 of SEQ ID No. 56, nt 940 to nt 1061 of SEQ ID No. 57, nt 940 to nt 1061 of SEQ ID No. 58, nt 940 to nt 1061 of SEQ ID No. 59, nt 941 to nt 1062 of SEQ ID No. 60, nt 941 to nt 1062 of SEQ ID No. 61, nt 940 to nt 1061 of SEQ ID No. 62, nt 940 to nt 1061 of SEQ ID No. 63, nt 941 to nt 1062 of SEQ ID No. 64, nt 940 to nt 1061 of SEQ ID No. 65, nt 940 to nt 1061 of SEQ ID No. 66, nt 941 to nt 1062 of SEQ ID No. 67, nt 941 to nt 1062 of SEQ ID No. 68, nt 940 to nt 1061 of SEQ ID No. 69, nt 940 to nt 1061 of SEQ ID No. 70, nt 941 to nt 1062 of SEQ ID No. 71, nt 940 to nt 1061 of SEQ ID No. 72, nt 941 to nt 1062 of SEQ ID No. 73, nt 941 to nt 1062 of SEQ ID No. 74, nt 940 to nt 1061 of SEQ ID No. 75, nt 940 to nt 1061 of SEQ ID No. 76, nt 940 to nt 1061 of SEQ ID No. 77, nt 940 to nt 1061 of SEQ ID No. 78, nt 941 to nt 1062 of SEQ ID No. 79, nt 941 to nt 1062 of SEQ ID No. 80, nt 940 to nt 1061 of SEQ ID No. 81, nt 940 to nt 1061 of SEQ ID No. 82, nt 940 to nt 1061 of SEQ ID No. 83, nt 940 to nt 1061 of SEQ ID No. 84, nt 941 to nt 1062 of SEQ ID No. 85, nt 941 to nt 1062 of SEQ ID No. 86, nt 941 to nt 1062 of SEQ ID No. 87, nt 941 to nt 1062 of SEQ ID No. 88, nt 941 to nt 1062 of SEQ ID No. 89, nt 940 to nt 1061 of SEQ ID No. 90, nt 940 to nt 1061 of SEQ ID No. 91, nt 940 to nt 1061 of SEQ ID No. 92, nt 941 to nt 1062 of SEQ ID No. 93, nt 940 to nt 1061 of SEQ ID No. 94, nt 941 to nt 1062 of SEQ ID No. 95, nt 941 to nt 1062 of SEQ ID No. 96, nt 941 to nt 1062 of SEQ ID No. 97, nt 941 to nt 1062 of SEQ ID No. 98, an allelic variant or a complementary sequence thereof.

Yet preferably the method of the invention further comprises determining the methylation status of at least one cytosine residue in the methylation relevant region of the LINE-1 repetitive sequence as in SEQ ID No. 220.

It is a further object of the invention an in vitro method of prognosing melanoma within a clinico-pathological stage thereof and/or monitoring the response to a melanoma therapy in a subject and/or to identify a subject to be treated with a DNA hypomethylating agent comprising determining the methylation status of at least one cytosine residue in a methylation relevant region of:

a] at least one of the following genes: ALOX12B, CTAG2, FGF4, IGLL1, OCIAD2, PAGES, SI00A9, SLC6A11, SLC6A18, TUB, BATF, BGN, CRHR1, CSAG1, CTSK, DENND2D, FLJ33860, GIBS, GRA14, LY96, MAGEA1, MAGEA10, MGC35206, MLSTD1, SLC18A2, SORBS2, and TRIM40; and/or

b] the LINE-1 repetitive sequence as in SEQ ID No. 220.

Preferably the melanoma is a cutaneous melanoma, preferably at stage III or IIIC.

Still preferably the method of the invention comprises the steps of:
(a) isolating DNA from a sample obtained from a subject;
(b) subjecting said DNA to methylation-sensitive sequence modification through incubation with suitable chemicals, or contacting said DNA with a reagent capable of selectively binding to methylated DNA;
(c) optionally amplifying said DNA as obtained in b), and
(d) determining the methylation status of said DNA.

It is a further object of the invention a kit for performing the method of the invention, comprising a chemical able to differentially modify DNA sequence depending on methylation status, or a reagent capable of selelctively binding to methylated DNA, and probes, primers and/or aptamers specific for each of genes as defined above.

Preferably the kit is in the form of an array. It may also be in the form of a microarray or microchip.

In the present invention, 6 genes are common between signatures A, B, C and D (Table 1) and 14 genes are common between signatures E, F and G (Table 2).

TABLE 1
Probes/sequences/genes shared among signatures A, B, C, D

| GeneSymbol | AccessionNumber | GeneID | IlmnID |
|---|---|---|---|
| GJB5 | NM_005268.2 | 2709 | cg01333788 |
| TUB | NM_177972.1 | 7275 | cg05492113 |
| CRHR1 | NM_004382.2 | 1394 | cg08929103 |
| TRIM40 | NM_138700.2 | 135644 | cg09196959 |
| FLJ33860 | NM_173644.1 | 284756 | cg14236389 |
| S100A9 | NM_002965.2 | 6280 | cg16139316 |

TABLE 2
Probes/sequences/genes shared among signatures E, F, G

| GeneSymbol | AccessionNumber | GeneID | IlmnID |
|---|---|---|---|
| DENND2D | NM_024901.3 | 79961 | cg00619207 |
| CTAG2 | NM_020994.2 | 30848 | cg01325515 |
| BGN | NM_001711.3 | 633 | cg04929865 |
| HCP5 | NM_006674.2 | 10866 | cg07971188 |
| CSAG1 | NM_153478.1 | 158511 | cg08977028 |
| C9orf90 | NM_197956.1 | 203245 | cg09296212 |
| CSAG1 | NM_153478.1 | 158511 | cg09917461 |
| RNASE1 | NM_198232.1 | 6035 | cg13718960 |
| HCP5 | NM_006674.2 | 10866 | cg13784557 |
| EMILIN1 | NM_007046.1 | 11117 | cg15895197 |
| COL5A2 | NM_000393.2 | 1290 | cg22774472 |
| LY96 | NM_015364.2 | 23643 | cg23732024 |
| MAGEA1 | NM_004988.3 | 4100 | cg23776892 |
| MAGEC2 | NM_016249.2 | 51438 | cg23813564 |

In the present invention, the “Methylation status” refers to the presence, absence and/or quantity of methylation at a particular nucleotide, or nucleotides within a portion of DNA. The methylation status of a particular DNA sequence (e.g., a methylation relevant region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.” A methylation value can be generated, for example, by quantifying the amount of methylated DNA by quantitative MSP. In this particular example, distinct primer pairs can be utilized to selectively amplify methylated or unmethylated target sequence. The amount of methylated and unmethylated DNA could be measured, for instance, by extrapolation of the values (e.g., the n. of PCR cycles at which the quantity of amplification product reaches a particular threshold level) obtained for the test sample against standard curves generated with different known amounts of methylated and unmethylated target sequences. Accordingly, a value, i.e., a methylation value, for example from the above described example, represents the methylation status and can thus be used as a quantitative indicator of methylation status. This is of particular use when it is desirable to compare the methylation status of a sequence in a sample to a threshold value.

The “methylation relevant region” is defined as a region containing CpG dinucleotides potential targets for DNA methylation which may extend from over 5000 bases 5′ to over 5000 bases 3′ respective to the putative transcription start site of the gene, and may include gene promoter(s), enhancer(s), exonic and intronic sequences.

As used in the present invention, the term “biologic sample” refers to any sample taken from a subject or derived from it, including, without limitation, any source of neoplastic cells from CM patients, such as primary cultures of autologous neoplastic cells, neoplastic cells purified from fresh autologous tumor tissues or from cryopreserved cell suspensions by immuno-sorting (e.g. using antibodies specific for cancer cells) or other means, cryopreserved tumor tissues, FFPE tumor tissues, or other types of fixed tumor tissue, micro-/macro-dissected tumor tissues, circulating neoplastic cells present in body fluids. The term further applies to biological fluids, including, without limitation, any fluid present in the subject in which acellular DNA or cancer cells may be present, such as blood, serum, plasma, bone marrow, cerebrospinal fluid, peritoneal/pleural fluid, lymph fluid, ascitic, serous fluid, sputum, lacrimal fluid, stool, and urine. Biological samples can be obtained from a subject using any of the methods known in the art.

TABLE 3
Signatures A, B, C, D, E, F and G associated with their
SEQ ID No. to be analyzed for methylation status.

| | | | | |
|---|---|---|---|---|
| | Gene | | | |
| Seq ID no. | Symbol | Accession N.* | Gene ID† | IlmnID§ |

Signature A

| | | | | |
|---|---|---|---|---|
| 1 | MGC29671 | NM_182538.3 | 201305 | cg01138020 |
| 2 | GJB5 | NM_005268.2 | 2709 | cg01333788 |
| 3 | KCNK16 | NM_032115.2 | 83795 | cg01669948 |
| 4 | IGLL1 | NM_020070.2 | 3543 | cg02415431 |
| 5 | ANXA8 | NM_001630.1 | 244 | cg03399459 |
| 6 | OR12D3 | NM_030959.2 | 81797 | cg03608577 |
| 7 | TUB | NM_177972.1 | 7275 | cg05492113 |
| 8 | CST11 | NM_080830.2 | 140880 | cg06489008 |
| 9 | TMEM10 | NM_033207.2 | 93377 | cg06825166 |
| 10 | CRHR1 | NM_004382.2 | 1394 | cg08929103 |
| 11 | TRIM40 | NM_138700.2 | 135644 | cg09196959 |
| 12 | GAS2L2 | NM_139285.1 | 246176 | cg10016608 |
| 13 | FOXN1 | NM_003593.2 | 8456 | cg10213812 |
| 14 | FXYD4 | NM_173160.2 | 53828 | cg12619162 |
| 15 | KRTHB2 | NM_033033.3 | 3888 | cg13696887 |
| 16 | FLJ33860 | NM_173644.1 | 284756 | cg14236389 |
| 17 | S100A9 | NM_002965.2 | 6280 | cg16139316 |
| 18 | FOXI1 | NM_012188.3 | 2299 | cg19233472 |
| 19 | GUCA2B | NM_007102.1 | 2981 | cg19728577 |
| 20 | CYP2B6 | NM_000767.4 | 1555 | cg19756068 |
| 21 | IL8RA | NM_000634.2 | 3577 | cg21004129 |
| 22 | CYP8B1 | NM_004391.1 | 1582 | cg21979032 |
| 23 | WFDC8 | NM_181510.1 | 90199 | cg22021786 |
| 24 | SLPI | NM_003064.2 | 6590 | cg23889010 |

Signature B

| | | | | |
|---|---|---|---|---|
| 25 | FLJ25421 | NM_152512.3 | 150350 | cg00044729 |
| 26 | LAMC2 | NM_005562.1 | 3918 | cg00208830 |
| 27 | C1orf36 | NM_183059.1 | 343035 | cg00658007 |
| 1 | MGC29671 | NM_182538.3 | 201305 | cg01138020 |
| 2 | GJB5 | NM_005268.2 | 2709 | cg01333788 |
| 3 | KCNK16 | NM_032115.2 | 83795 | cg01669948 |
| 28 | ANGPTL3 | NM_014495.2 | 27329 | cg02218214 |
| 4 | IGLL1 | NM_020070.2 | 3543 | cg02415431 |
| 29 | DEFB4 | NM_004942.2 | 1673 | cg02658251 |
| 30 | STK38 | NM_007271.2 | 11329 | cg02964385 |
| 31 | SEMG2 | NM_003008.2 | 6407 | cg03312792 |
| 5 | ANXA8 | NM_001630.1 | 244 | cg03399459 |
| 6 | OR12D3 | NM_030959.2 | 81797 | cg03608577 |
| 32 | PREPL | NM_006036.2 | 9581 | cg03894103 |
| 33 | IL17F | NM_052872.3 | 112744 | cg04063348 |
| 34 | DCLRE1A | NM_014881.2 | 9937 | cg04281204 |
| 35 | FLJ20581 | NM_017888.2 | 54988 | cg04428453 |
| 36 | PTCD2 | NM_024754.2 | 79810 | cg04527989 |
| 37 | CX3CR1 | NM_001337.3 | 1524 | cg04569233 |
| 7 | TUB | NM_177972.1 | 7275 | cg05492113 |
| 38 | UBE2E1 | NM_182666.1 | 7324 | cg06282059 |
| 8 | CST11 | NM_080830.2 | 140880 | cg06489008 |
| 39 | IFT140 | NM_014714.2 | 9742 | cg06730286 |
| 9 | TMEM10 | NM_033207.2 | 93377 | cg06825166 |
| 40 | SLC22A8 | NM_004254.2 | 9376 | cg06917325 |
| 41 | MLN | NM_002418.1 | 4295 | cg08332212 |
| 42 | DIRC1 | NM_052952.1 | 116093 | cg08654334 |
| 43 | C1orf64 | NM_178840.2 | 149563 | cg08887581 |
| 10 | CRHR1 | NM_004382.2 | 1394 | cg08929103 |
| 11 | TRIM40 | NM_138700.2 | 135644 | cg09196959 |
| 44 | TNFSF13B | NM_006573.3 | 10673 | cg09646392 |
| 12 | GAS2L2 | NM_139285.1 | 246176 | cg10016608 |
| 13 | FOXN1 | NM_003593.2 | 8456 | cg10213812 |
| 45 | CYP2B6 | NM_000767.4 | 1555 | cg10322876 |
| 46 | ACSL5 | NM_016234.3 | 51703 | cg11152574 |
| 47 | SERPINB12 | NM_080474.1 | 89777 | cg11435943 |
| 48 | APOBEC4 | NM_203454.1 | 403314 | cg11505048 |
| 49 | TNNI1 | NM_003281.3 | 7135 | cg12114524 |
| 50 | C1orf42 | NM_019060.1 | 54544 | cg12588301 |
| 14 | FXYD4 | NM_173160.2 | 53828 | cg12619162 |
| 51 | PPAT | NM_002703.3 | 5471 | cg13086586 |
| 52 | PSD4 | NM_012455.2 | 23550 | cg13273136 |
| 53 | SCN4A | NM_000334.3 | 6329 | cg13694749 |
| 15 | KRTHB2 | NM_033033.3 | 3888 | cg13696887 |
| 54 | IL8RB | NM_001557.2 | 3579 | cg13739417 |
| 55 | UNC45B | NM_173167.2 | 146862 | cg13859324 |
| 56 | C1orf201 | NM_178122.2 | 90529 | cg13980834 |
| 57 | SFTPG | NM_205854.1 | 389376 | cg14034870 |
| 58 | LOC124751 | NM_213597.1 | 124751 | cg14069619 |
| 16 | FLJ33860 | NM_173644.1 | 284756 | cg14236389 |
| 59 | ANKRD1 | NM_014391.2 | 27063 | cg14558138 |
| 60 | FMO2 | NM_001460.2 | 2327 | cg14721213 |
| 61 | ARSG | NM_014960.2 | 22901 | cg15308737 |
| 62 | GJB4 | NM_153212.1 | 127534 | cg15554401 |
| 17 | S100A9 | NM_002965.2 | 6280 | cg16139316 |
| 63 | LRRN5 | NM_006338.2 | 10446 | cg16456919 |
| 64 | MSMB | NM_002443.2 | 4477 | cg17030820 |
| 65 | HLA-DOB | NM_002120.2 | 3112 | cg17103109 |
| 66 | R3HDML | NM_178491.2 | 140902 | cg17692403 |
| 67 | SPINK5 | NM_006846.2 | 11005 | cg17788013 |
| 68 | DLG5 | NM_004747.2 | 9231 | cg17791617 |
| 69 | PALLD | NM_016081.2 | 23022 | cg17925436 |
| 70 | WFDC12 | NM_080869.1 | 128488 | cg18241647 |
| 71 | WDR20 | NM_181291.1 | 91833 | cg18412984 |
| 72 | FLJ32771 | NM_145017.1 | 220004 | cg18432384 |
| 73 | PKHD1 | NM_138694.2 | 5314 | cg18885346 |
| 18 | FOXI1 | NM_012188.3 | 2299 | cg19233472 |
| 74 | DEFB123 | NM_153324.2 | 245936 | cg19241311 |
| 75 | KRTHB3 | NM_002282.2 | 3889 | cg19258973 |
| 19 | GUCA2B | NM_007102.1 | 2981 | cg19728577 |
| 20 | CYP2B6 | NM_000767.4 | 1555 | cg19756068 |
| 76 | BSND | NM_057176.2 | 7809 | cg19971655 |
| 77 | NALP10 | NM_176821.2 | 338322 | cg20311730 |
| 78 | NR5A2 | NM_003822.3 | 2494 | cg20406878 |
| 79 | KRT25D | NM_181535.2 | 162605 | cg20484002 |
| 21 | IL8RA | NM_000634.2 | 3577 | cg21004129 |
| 80 | APOD | NM_001647.2 | 347 | cg21084260 |
| 81 | CLEC4F | NM_173535.2 | 165530 | cg21148892 |
| 82 | THRB | NM_000461.2 | 7068 | cg21303011 |
| 83 | KRTHB5 | NM_002283.2 | 3891 | cg21518208 |
| 22 | CYP8B1 | NM_004391.1 | 1582 | cg21979032 |
| 23 | WFDC8 | NM_181510.1 | 90199 | cg22021786 |
| 84 | SLC13A1 | NM_022444.3 | 6561 | cg22024657 |
| 85 | C3orf27 | NM_007354.1 | 23434 | cg22068211 |
| 86 | MGC35206 | NM_178552.2 | 339669 | cg22088368 |
| 87 | C3orf63 | NM_015224.1 | 23272 | cg22332306 |
| 88 | DARC | NM_002036.2 | 2532 | cg23507131 |
| 24 | SLPI | NM_003064.2 | 6590 | cg23889010 |
| 89 | SLC2A7 | NM_207420.1 | 155184 | cg24027679 |
| 90 | CPA3 | NM_001870.1 | 1359 | cg24290574 |
| 91 | MIS12 | NM_024039.1 | 79003 | cg24445405 |
| 92 | KRTHB4 | NM_033045.2 | 3890 | cg24461814 |
| 93 | MRVI1 | NM_130385.1 | 10335 | cg24541550 |
| 94 | MIA2 | NM_054024.3 | 117153 | cg24603941 |
| 95 | CYP2A7 | NM_030589.2 | 1549 | cg25427638 |
| 96 | ANGPT4 | NM_015985.2 | 51378 | cg26540515 |
| 97 | SLC44A5 | NM_152697.2 | 204962 | cg27336379 |
| 98 | CST9L | NM_080610.1 | 128821 | cg27655855 |

Signature C

| | | | | |
|---|---|---|---|---|
| 99 | SLC18A2 | NM_003054.2 | 6571 | cg00498305 |
| 2 | GJB5 | NM_005268.2 | 2709 | cg01333788 |
| 100 | SLC6A18 | NM_182632.1 | 348932 | cg02064402 |
| 4 | IGLL1 | NM_020070.2 | 3543 | cg02415431 |
| 101 | ALOX12B | NM_001139.1 | 242 | cg03742272 |
| 102 | WNT10B | NM_003394.2 | 7480 | cg05164634 |
| 7 | TUB | NM_177972.1 | 7275 | cg05492113 |
| 103 | S100A9 | NM_002965.2 | 6280 | cg07039113 |
| 10 | CRHR1 | NM_004382.2 | 1394 | cg08929103 |
| 11 | TRIM40 | NM_138700.2 | 135644 | cg09196959 |
| 104 | SLC6A11 | NM_014229.1 | 6538 | cg09395732 |
| 16 | FLJ33860 | NM_173644.1 | 284756 | cg14236389 |
| 105 | FGF4 | NM_002007.1 | 2249 | cg14578030 |
| 17 | S100A9 | NM_002965.2 | 6280 | cg16139316 |
| 86 | MGC35206 | NM_178552.2 | 339669 | cg22088368 |
| 106 | PCDHAC2 | NM_031883.2 | 56134 | cg24076884 |
| 107 | GRM4 | NM_000841.1 | 2914 | cg26424956 |

Signature D

| | | | | |
|---|---|---|---|---|
| 25 | FLJ25421 | NM_152512.3 | 150350 | cg00044729 |
| 108 | ANXA13 | NM_004306.2 | 312 | cg00283535 |
| 99 | SLC18A2 | NM_003054.2 | 6571 | cg00498305 |
| 109 | CTAG2 | NM_020994.2 | 30848 | cg01325515 |
| 2 | GJB5 | NM_005268.2 | 2709 | cg01333788 |
| 3 | KCNK16 | NM_032115.2 | 83795 | cg01669948 |
| 100 | SLC6A18 | NM_182632.1 | 348932 | cg02064402 |
| 30 | STK38 | NM_007271.2 | 11329 | cg02964385 |
| 110 | PVALB | NM_002854.2 | 5816 | cg02978737 |
| 6 | OR12D3 | NM_030959.2 | 81797 | cg03608577 |
| 101 | ALOX12B | NM_001139.1 | 242 | cg03742272 |
| 111 | HAO2 | NM_016527.2 | 51179 | cg03762535 |
| 34 | DCLRE1A | NM_014881.2 | 9937 | cg04281204 |
| 102 | WNT10B | NM_003394.2 | 7480 | cg05164634 |
| 7 | TUB | NM_177972.1 | 7275 | cg05492113 |
| 38 | UBE2E1 | NM_182666.1 | 7324 | cg06282059 |
| 39 | IFT140 | NM_014714.2 | 9742 | cg06730286 |
| 112 | SLC4A7 | NM_003615.2 | 9497 | cg06798189 |
| 103 | S100A9 | NM_002965.2 | 6280 | cg07039113 |
| 42 | DIRC1 | NM_052952.1 | 116093 | cg08654334 |
| 10 | CRHR1 | NM_004382.2 | 1394 | cg08929103 |
| 11 | TRIM40 | NM_138700.2 | 135644 | cg09196959 |
| 104 | SLC6A11 | NM_014229.1 | 6538 | cg09395732 |
| 113 | TDRD5 | NM_173533.2 | 163589 | cg09656934 |
| 46 | ACSL5 | NM_016234.3 | 51703 | cg11152574 |
| 48 | APOBEC4 | NM_203454.1 | 403314 | cg11505048 |
| 114 | FAAH | NM_001441.1 | 2166 | cg12671744 |
| 115 | CD1D | NM_001766.2 | 912 | cg13765621 |
| 56 | C1orf201 | NM_178122.2 | 90529 | cg13980834 |
| 16 | FLJ33860 | NM_173644.1 | 284756 | cg14236389 |
| 59 | ANKRD1 | NM_014391.2 | 27063 | cg14558138 |
| 105 | FGF4 | NM_002007.1 | 2249 | cg14578030 |
| 116 | FLJ39501 | NM_173483.1 | 126410 | cg14851685 |
| 17 | S100A9 | NM_002965.2 | 6280 | cg16139316 |
| 63 | LRRN5 | NM_006338.2 | 10446 | cg16456919 |
| 117 | GLT1D1 | NM_144669.1 | 144423 | cg16717225 |
| 118 | IL28B | NM_172139.2 | 282617 | cg17982671 |
| 119 | AVPR1B | NM_000707.2 | 553 | cg18992688 |
| 120 | GJB3 | NM_024009.2 | 2707 | cg21151355 |
| 86 | MGC35206 | NM_178552.2 | 339669 | cg22088368 |
| 106 | PCDHAC2 | NM_031883.2 | 56134 | cg24076884 |
| 121 | GABRG3 | NM_033223.1 | 2567 | cg24244000 |
| 94 | MIA2 | NM_054024.3 | 117153 | cg24603941 |
| 122 | CTAG1B | NM_001327.1 | 1485 | cg25531166 |
| 123 | UNQ9433 | NM_207413.1 | 389658 | cg26021627 |
| 107 | GRM4 | NM_000841.1 | 2914 | cg26424956 |
| 124 | CEACAM6 | NM_002483.3 | 4680 | cg26813458 |

Signature E

| | | | | |
|---|---|---|---|---|
| 125 | DENND2D | NM_024901.3 | 79961 | cg00619207 |
| 109 | CTAG2 | NM_020994.2 | 30848 | cg01325515 |
| 126 | PGCP | NM_016134.2 | 10404 | cg01892689 |
| 127 | KCNG1 | NM_002237.2 | 3755 | cg02305261 |
| 128 | FAM107B | NM_031453.2 | 83641 | cg02876062 |
| 129 | FANCE | NM_021922.2 | 2178 | cg03030757 |
| 130 | FAM38B | NM_022068.1 | 63895 | cg03426225 |
| 131 | FOXF2 | NM_001452.1 | 2295 | cg03848675 |
| 132 | BGN | NM_001711.3 | 633 | cg04929865 |
| 133 | MAGEA10 | NM_021048.3 | 4109 | cg04950711 |
| 134 | SPATS1 | NM_145026.2 | 221409 | cg05010967 |
| 135 | PRRX1 | NM_022716.2 | 5396 | cg05593300 |
| 136 | MLSTD1 | NM_018099.3 | 55711 | cg05697976 |
| 137 | PDZD4 | NM_032512.2 | 57595 | cg06051662 |
| 138 | HCP5 | NM_006674.2 | 10866 | cg07971188 |
| 139 | OCIAD2 | NM_152398.2 | 132299 | cg08942875 |
| 140 | CSAG1 | NM_153478.1 | 158511 | cg08977028 |
| 141 | SLCO4A1 | NM_016354.3 | 28231 | cg09210315 |
| 142 | C9orf90 | NM_197956.1 | 203245 | cg09296212 |
| 143 | CSAG1 | NM_153478.1 | 158511 | cg09917461 |
| 144 | AMPH | NM_001635.2 | 273 | cg09966445 |
| 145 | MT1G | NM_005950.1 | 4495 | cg09985279 |
| 146 | C20orf100 | NM_032883.1 | 84969 | cg11319389 |
| 147 | CST7 | NM_003650.2 | 8530 | cg11804789 |
| 148 | KIAA0367 | NM_015225.1 | 23273 | cg11880010 |
| 149 | RNASE1 | NM_198232.1 | 6035 | cg13718960 |
| 150 | HCP5 | NM_006674.2 | 10866 | cg13784557 |
| 151 | NDN | NM_002487.2 | 4692 | cg13828758 |
| 152 | RAC2 | NM_002872.3 | 5880 | cg14072120 |
| 153 | AXL | NM_021913.2 | 558 | cg14892768 |
| 154 | BATF | NM_006399.2 | 10538 | cg15645309 |
| 155 | EMILIN1 | NM_007046.1 | 11117 | cg15895197 |
| 156 | ARHGEF7 | NM_003899.2 | 8874 | cg16427670 |
| 157 | EVL | NM_016337.2 | 51466 | cg17813891 |
| 158 | RAC2 | NM_002872.3 | 5880 | cg18265887 |
| 159 | KIAA0367 | NM_015225.1 | 23273 | cg19282250 |
| 160 | TFF3 | NM_003226.2 | 7033 | cg20488657 |
| 161 | CTSK | NM_000396.2 | 1513 | cg20802392 |
| 162 | C21orf70 | NM_058190.2 | 85395 | cg20884362 |
| 163 | PRKCZ | NM_002744.4 | 5590 | cg21137823 |
| 164 | MLSTD1 | NM_018099.3 | 55711 | cg21522988 |
| 165 | COL5A2 | NM_000393.2 | 1290 | cg22774472 |
| 166 | LY96 | NM_015364.2 | 23643 | cg23732024 |
| 167 | MAGEA1 | NM_004988.3 | 4100 | cg23776892 |
| 168 | MAGEC2 | NM_016249.2 | 51438 | cg23813564 |
| 169 | DENND2D | NM_024901.3 | 79961 | cg24641737 |
| 170 | PPIE | NM_006112.2 | 10450 | cg24920358 |
| 171 | SORBS2 | NM_021069.3 | 8470 | cg26583078 |
| 172 | PROCR | NM_006404.3 | 10544 | cg26806924 |
| 173 | TMEPAI | NM_020182.3 | 56937 | cg26912636 |
| 174 | SSNA1 | NM_003731.1 | 8636 | cg27291231 |
| 175 | ZNF324 | NM_014347.1 | 25799 | cg27401095 |

Signature F

| | | | | |
|---|---|---|---|---|
| 25 | FLJ25421 | NM_152512.3 | 150350 | cg00044729 |
| 125 | DENND2D | NM_024901.3 | 79961 | cg00619207 |
| 109 | CTAG2 | NM_020994.2 | 30848 | cg01325515 |
| 126 | PGCP | NM_016134.2 | 10404 | cg01892689 |
| 127 | KCNG1 | NM_002237.2 | 3755 | cg02305261 |
| 175 | MGMT | NT_008818.15 | 4255 | cg02330106 |
| 128 | FAM107B | NM_031453.2 | 83641 | cg02876062 |
| 129 | FANCE | NM_021922.2 | 2178 | cg03030757 |
| 176 | GAGE2 | NM_001472.1 | 2574 | cg04431776 |
| 177 | C11orf1 | NM_022761.1 | 64776 | cg04545708 |
| 178 | ABCB4 | NM_018849.1 | 5244 | cg04737046 |
| 132 | BGN | NM_001711.3 | 633 | cg04929865 |
| 133 | MAGEA10 | NM_021048.3 | 4109 | cg04950711 |
| 134 | SPATS1 | NM_145026.2 | 221409 | cg05010967 |
| 179 | CTNNAL1 | NM_003798.1 | 8727 | cg05485060 |
| 135 | PRRX1 | NM_022716.2 | 5396 | cg05593300 |
| 180 | EMP1 | NM_001423.1 | 2012 | cg05885720 |
| 181 | RNASE1 | NM_198232.1 | 6035 | cg05958352 |
| 137 | PDZD4 | NM_032512.2 | 57595 | cg06051662 |
| 182 | MAGEA6 | NM_005363.2 | 4105 | cg06899808 |
| 183 | CHMP4A | NM_014169.2 | 29082 | cg07296772 |
| 184 | ADORA2B | NM_000676.2 | 136 | cg07677850 |
| 138 | HCP5 | NM_006674.2 | 10866 | cg07971188 |
| 185 | PLA1A | NM_015900.1 | 51365 | cg08141463 |
| 186 | KIAA0141 | NM_014773.2 | 9812 | cg08158289 |
| 187 | OLFM1 | NM_014279.2 | 10439 | cg08268099 |
| 188 | ERO1L | NM_014584.1 | 30001 | cg08554114 |
| 140 | CSAG1 | NM_153478.1 | 158511 | cg08977028 |
| 141 | SLCO4A1 | NM_016354.3 | 28231 | cg09210315 |
| 189 | CXorf12 | NM_003492.1 | 8269 | cg09275137 |
| 142 | C9orf90 | NM_197956.1 | 203245 | cg09296212 |
| 143 | CSAG1 | NM_153478.1 | 158511 | cg09917461 |
| 144 | AMPH | NM_001635.2 | 273 | cg09966445 |
| 145 | MT1G | NM_005950.1 | 4495 | cg09985279 |
| 190 | ARHGDIB | NM_001175.4 | 397 | cg10925082 |
| 146 | C20orf100 | NM_032883.1 | 84969 | cg11319389 |
| 147 | CST7 | NM_003650.2 | 8530 | cg11804789 |
| 148 | KIAA0367 | NM_015225.1 | 23273 | cg11880010 |
| 149 | RNASE1 | NM_198232.1 | 6035 | cg13718960 |
| 150 | HCP5 | NM_006674.2 | 10866 | cg13784557 |
| 151 | NDN | NM_002487.2 | 4692 | cg13828758 |
| 152 | RAC2 | NM_002872.3 | 5880 | cg14072120 |
| 153 | AXL | NM_021913.2 | 558 | cg14892768 |
| 191 | GRAMD3 | NM_023927.1 | 65983 | cg15165084 |
| 61 | ARSG | NM_014960.2 | 22901 | cg15308737 |
| 192 | PLD1 | NM_002662.2 | 5337 | cg15329866 |
| 193 | HNMT | NM_006895.2 | 3176 | cg15441973 |
| 194 | ZNF558 | NM_144693.1 | 148156 | cg15751406 |
| 195 | ATP6V1E2 | NM_080653.3 | 90423 | cg15759873 |
| 155 | EMILIN1 | NM_007046.1 | 11117 | cg15895197 |
| 156 | ARHGEF7 | NM_003899.2 | 8874 | cg16427670 |
| 196 | KRT25C | NM_181537.2 | 342574 | cg16488522 |
| 197 | ZNF532 | NM_018181.4 | 55205 | cg17675150 |
| 157 | EVL | NM_016337.2 | 51466 | cg17813891 |
| 158 | RAC2 | NM_002872.3 | 5880 | cg18265887 |
| 198 | SLAMF9 | NM_033438.1 | 89886 | cg18913171 |
| 199 | NBL1 | NM_005380.3 | 4681 | cg19136075 |
| 159 | KIAA0367 | NM_015225.1 | 23273 | cg19282250 |
| 200 | MAGEA10 | NM_021048.3 | 4109 | cg19964192 |
| 160 | TFF3 | NM_003226.2 | 7033 | cg20488657 |
| 162 | C21orf70 | NM_058190.2 | 85395 | cg20884362 |
| 80 | APOD | NM_001647.2 | 347 | cg21084260 |
| 201 | ABCA8 | NM_007168.2 | 10351 | cg21660392 |
| 202 | ACOX2 | NM_003500.2 | 8309 | cg22012981 |
| 165 | COL5A2 | NM_000393.2 | 1290 | cg22774472 |
| 166 | LY96 | NM_015364.2 | 23643 | cg23732024 |
| 167 | MAGEA1 | NM_004988.3 | 4100 | cg23776892 |
| 168 | MAGEC2 | NM_016249.2 | 51438 | cg23813564 |
| 203 | TIMP3 | NM_000362.4 | 7078 | cg24080529 |
| 170 | PPIE | NM_006112.2 | 10450 | cg24920358 |
| 204 | RPUSD3 | NM_173659.2 | 285367 | cg26417554 |
| 172 | PROCR | NM_006404.3 | 10544 | cg26806924 |
| 173 | TMEPAI | NM_020182.3 | 56937 | cg26912636 |

Signature G

| | | | | |
|---|---|---|---|---|
| 205 | PRDX2 | NM_005809.4 | 7001 | cg00155609 |
| 125 | DENND2D | NM_024901.3 | 79961 | cg00619207 |
| 109 | CTAG2 | NM_020994.2 | 30848 | cg01325515 |
| 206 | PCOLCE | NM_002593.2 | 5118 | cg02797569 |
| 132 | BGN | NM_001711.3 | 633 | cg04929865 |
| 207 | OCIAD2 | NM_152398.2 | 132299 | cg04961553 |
| 181 | RNASE1 | NM_198232.1 | 6035 | cg05958352 |
| 182 | MAGEA6 | NM_005363.2 | 4105 | cg06899808 |
| 138 | HCP5 | NM_006674.2 | 10866 | cg07971188 |
| 139 | OCIAD2 | NM_152398.2 | 132299 | cg08942875 |
| 140 | CSAG1 | NM_153478.1 | 158511 | cg08977028 |
| 142 | C9orf90 | NM_197956.1 | 203245 | cg09296212 |
| 143 | CSAG1 | NM_153478.1 | 158511 | cg09917461 |
| 208 | CTSK | NM_000396.2 | 1513 | cg11946165 |
| 209 | ADM | NM_001124.1 | 133 | cg12320306 |
| 149 | RNASE1 | NM_198232.1 | 6035 | cg13718960 |
| 150 | HCP5 | NM_006674.2 | 10866 | cg13784557 |
| 210 | PAGE5 | NM_130467.3 | 90737 | cg13991029 |
| 211 | NNMT | NM_006169.2 | 4837 | cg14209518 |
| 155 | EMILIN1 | NM_007046.1 | 11117 | cg15895197 |
| 212 | MAGEA3 | NM_005362.3 | 4102 | cg16390856 |
| 213 | RAB11FIP5 | NM_015470.1 | 26056 | cg18943195 |
| 214 | ARHGDIB | NM_001175.4 | 397 | cg19826026 |
| 215 | EMILIN1 | NM_007046.1 | 11117 | cg20516209 |
| 216 | LPXN | NM_004811.1 | 9404 | cg20654468 |
| 161 | CTSK | NM_000396.2 | 1513 | cg20802392 |
| 217 | ALDH1A3 | NM_000693.1 | 220 | cg21359747 |
| 218 | SUHW1 | NM_080740.3 | 129025 | cg21604856 |
| 201 | ABCA8 | NM_007168.2 | 10351 | cg21660392 |
| 165 | COL5A2 | NM_000393.2 | 1290 | cg22774472 |
| 166 | LY96 | NM_015364.2 | 23643 | cg23732024 |
| 167 | MAGEA1 | NM_004988.3 | 4100 | cg23776892 |
| 168 | MAGEC2 | NM_016249.2 | 51438 | cg23813564 |
| 219 | GSTT1 | NM_000853.1 | 2952 | cg24330042 |
| 169 | DENND2D | NM_024901.3 | 79961 | cg24641737 |

*GeneBank accession number,
†Illumina unique gene identifyer,
§Illumina unique probe identifier that defines the exact CpG dinucleotide that is evaluated.

TABLE 4
Genes of the various signatures and relative SEQ ID No. Genomic coordinates are
given respective to assembly GRCh37, in the form of: assembly n: chromosome n: start
position-end position

Seq ID no. 1, MGC29671 - GRCh37:17: 4336094-4338094, Probe design region
(genomic position GRCh37:17: 4337034-4337155, nt 941 to nt 1062 of SEQ ID No. 1),
investigated CpG (genomic position GRCh37:17: 4337094, nt 1001 of SEQ ID No. 1)
Seq ID no. 2, GJB5 - GRCh37:1: 35219815-35221815: 1, Probe design region (genomic
position GRCh37:1: 35220754-35220875, nt 940 to nt 1061 of SEQ ID No. 2),
investigated CpG (genomic position GRCh37:1: 35220814, nt 1000 of SEQ ID No. 2)
Seq ID no. 3, KCNK16 - GRCh37:6: 39289878-39291878, Probe design region (genomic
position GRCh37:6: 39290817-39290938, nt 940 to nt 1061 of SEQ ID No. 3),
investigated CpG (genomic position GRCh37:6: 39290877, nt 1000 of SEQ ID No. 3)
Seq ID no. 4, IGLL1 - GRCh37:22: 23922255-23924255, Probe design region (genomic
position GRCh37:22: 23923195-23923316, nt 941 to nt 1062 of SEQ ID No. 4),
investigated CpG (genomic position GRCh37:22: 23923255, nt 1001 of SEQ ID No. 4)
Seq ID no. 5, ANXA8 - GRCh37:10: 47746083-47748083, Probe design region (genomic
position GRCh37:10: 47747022-47747143, nt 940 to nt 1061 of SEQ ID No. 5),
investigated CpG (genomic position GRCh37:10: 47747083, nt 1000 of SEQ ID No. 5)
Seq ID no. 6, OR12D3 - GRCh37:6: 29341680-29343680, Probe design region (genomic
position GRCh37:6: 29342619-29342740, nt 940 to nt 1061 of SEQ ID No. 6),
investigated CpG (genomic position GRCh37:6: 29342680, nt 1000 of SEQ ID No. 6)
Seq ID no. 7, TUB - GRCh37:11: 8059333-8061333, Probe design region (genomic
position GRCh37:11: 8060272-8060393, nt 940 to nt 1061 of SEQ ID No. 7), investigated
CpG (genomic position GRCh37:11: 8060333, nt 1000 of SEQ ID No. 7)
Seq ID no. 8, CST11 - GRCh37:20: 23432349-23434349 - Probe design region (genomic
position GRCh37:11: 8060272-8060393, nt 941 to nt 1062 of SEQ ID No. 8), investigated
CpG (genomic position GRCh37:20: 23433349, nt 1001 of SEQ ID No. 8)
Seq ID no. 9, TMEM10 - GRCh37:10: 98118079-98120079, Probe design region
(genomic position GRCh37:11: 8060272-8060393, nt 941 to nt 1062 of SEQ ID No. 9),
investigated CpG (genomic position GRCh37:10: 98119079, nt 1001 of SEQ ID No. 9)
Seq ID no. 10, CRHR1 - GRCh37:17: 43859355-43861355, Probe design region
(genomic position GRCh37:17: 43860295-43860416, nt 941 to nt 1062 of SEQ ID No.
10), investigated CpG (genomic position GRCh37:17: 43860355, nt 1001 of SEQ ID No.
10)
Seq ID no. 11, TRIM40 - GRCh37:6: 30103441-30105441, Probe design region (genomic
position GRCh37:6: 30104380-30104501, nt 940 to nt 1061 of SEQ ID No. 11),
investigated CpG (genomic position GRCh37:6: 30104441, nt 1000 of SEQ ID No. 11)
Seq ID no. 12, GAS2L2 - GRCh37:17: 34078666-34080666, Probe design region
(genomic position GRCh37:17: 34079605-34079726, nt 940 to nt 1061 of SEQ ID No.
12), investigated CpG (genomic position GRCh37:17: 34079666, nt 1000 of SEQ ID No.
12)
Seq ID no. 13, FOXN1 - GRCh37:17: 26849322-26851322, Probe design region (genomic
position GRCh37:17: 26850262-26850383, nt 941 to nt 1062 of SEQ ID No. 13),
investigated CpG (genomic position GRCh37:17: 26850322, nt 1001 of SEQ ID No. 13)
Seq ID no. 14, FXYD4 - GRCh37:10: 43866156-43868156, Probe design region (genomic
position GRCh37:10: 43867095-43867216, nt 940 to nt 1061 of SEQ ID No. 14),
investigated CpG (genomic position GRCh37:10: 43867156, nt 1000 of SEQ ID No. 14)
Seq ID no. 15, KRTHB2 - GRCh37:12: 52799423-52801423, Probe design region
(genomic position GRCh37:12: 52800363-52800484, nt 941 to nt 1062 of SEQ ID No.
15), investigated CpG (genomic position GRCh37:12: 52800423, nt 1001 of SEQ ID No.
15)
Seq ID no. 16, FLJ33860 - GRCh37:20: 58630039-58632039, Probe design region
(genomic position GRCh37: 20: 58630978-58631099, nt 940 to nt 1061 of SEQ ID No.
16), investigated CpG (genomic position GRCh37:20: 58631039, nt 1000 of SEQ ID No.
16)
Seq ID no. 17, S100A9 - GRCh37:1: 153329759-153331759, Probe design region
(genomic position GRCh37:1: 153330698-153330819, nt 940 to nt 1061 of SEQ ID No.
17), investigated CpG (genomic position GRCh37:1: 153330759, nt 1000 of SEQ ID No.
17)
Seq ID no. 18, FOXI1 - GRCh37:5: 169531752-169533752, Probe design region
(genomic position GRCh37:5: 169532692-169532813, nt 941 to nt 1062 of SEQ ID No.
18), investigated CpG (genomic position GRCh37:5: 169532752, nt 1001 of SEQ ID No.
18)
Seq ID no. 19, GUCA2B - GRCh37:1: 42618164-42620164, Probe design region
(genomic position GRCh37:1: 42619103-42619224, nt 940 to nt 1061 of SEQ ID No. 19),
investigated CpG (genomic position GRCh37:1: 42619164, nt 1000 of SEQ ID No. 19)
Seq ID no. 20, CYP2B6 - GRCh37:19: 41496223-41498223, Probe design region
(genomic position GRCh37:19: 41497162-41497283, nt 940 to nt 1061 of SEQ ID No.
20), investigated CpG (genomic position GRCh37:19: 41497223, nt 1000 of SEQ ID No.
20)
Seq ID no. 21, IL8RA - GRCh37:2: 219031025-219033025, Probe design region
(genomic position GRCh37:2: 219031964-219032085, nt 940 to nt 1061 of SEQ ID No.
21), investigated CpG (genomic position GRCh37:2: 219032025, nt 1000 of SEQ ID No.
21)
Seq ID no. 22, CYP8B1 - GRCh37:3: 42916518-42918518, Probe design region (genomic
position GRCh37:3: 42917457-42917578, nt 940 to nt 1061 of SEQ ID No. 22),
investigated CpG (genomic position GRCh37:3: 42917518, nt 1000 of SEQ ID No. 22)
Seq ID no. 23, WFDC8 - GRCh37:20: 44206289-44208289, Probe design region
(genomic position GRCh37:20: 44206289-44208289, nt 940 to nt 1061 of SEQ ID No.
23), investigated CpG (genomic position GRCh37:20: 44207289, nt 1000 of SEQ ID No.
23)
Seq ID no. 24, SLPI - GRCh37:20: 43881991-43883991, Probe design region (genomic
position GRCh37: 20: 43882933-43883051, nt 940 to nt 1061 of SEQ ID No. 24),
investigated CpG (genomic position GRCh37:20: 43882991, nt 1000 of SEQ ID No. 24)
Seq ID no. 25, FLJ25421 - GRCh37:22: 40289977-40291977, Probe design region
(genomic position GRCh37:22: 40290916-40291037, nt 940 to nt 1061 of SEQ ID No.
25), investigated CpG (genomic position GRCh37:22: 40290977, nt 1000 of SEQ ID No.
25)
Seq ID no. 26, LAMC2 - GRCh37:1: 183153778-183155778, Probe design region
(genomic position GRCh37:1: 183154718-183154839, nt 941 to nt 1062 of SEQ ID No.
26), investigated CpG (genomic position GRCh37:1: 183154778, nt 1001 of SEQ ID No.
26)
Seq ID no. 27, C1orf36 - GRCh37:1: 211665129-211667129, Probe design region
(genomic position GRCh37:1: 211666069-211666190, nt 941 to nt 1062 of SEQ ID No.
27), investigated CpG (genomic position GRCh37:1: 211666129, nt 1001 of SEQ ID No.
27)
Seq ID no. 28, ANGPTL3 - GRCh37:1: 63061489-63063489, Probe design region
(genomic position GRCh37:1: 63062429-63062550, nt 941 to nt 1062 of SEQ ID No. 28),
investigated CpG (genomic position GRCh37:1: 63062489, nt 1001 of SEQ ID No. 28)
Seq ID no. 29, DEFB4 - GRCh37:8: 7750367-7752367, Probe design region (genomic
position GRCh37:8: 7751306-7751427, nt 940 to nt 1061 of SEQ ID No. 29),
investigated CpG (genomic position GRCh37:8: 7751367, nt 1000 of SEQ ID No. 29)
Seq ID no. 30, STK38 - GRCh37:6: 36514677-36516677, Probe design region (genomic
position GRCh37:6: 36515616-36515737, nt 940 to nt 1061 of SEQ ID No. 30),
investigated CpG (genomic position GRCh37:6: 36515677, nt 1000 of SEQ ID No. 30)
Seq ID no. 31, SEMG2 - GRCh37:20: 43848760-43850760, Probe design region
(genomic position GRCh37:20: 43849699-43849820, nt 940 to nt 1061 of SEQ ID No.
31), investigated CpG (genomic position GRCh37:20: 43849760, nt 1000 of SEQ ID No.
31)
Seq ID no. 32, PREPL - GRCh37:2: 44585902-44587902, Probe design region (genomic
position GRCh37:2: 44586841-44586962, nt 940 to nt 1061 of SEQ ID No. 32),
investigated CpG (genomic position GRCh37:2: 44586902, nt 1000 of SEQ ID No. 32)
Seq ID no. 33, IL17F - GRCh37:6: 52108348-52110348, Probe design region (genomic
position GRCh37:6: 52109287-52109408, nt 940 to nt 1061 of SEQ ID No. 33),
investigated CpG (genomic position GRCh37:6: 52109348, nt 1000 of SEQ ID No. 33)
Seq ID no. 34, DCLRE1A - GRCh37:10: 115611803-115613803, Probe design region
(genomic position GRCh37:10: 115612742-115612863, nt 940 to nt 1061 of SEQ ID No.
34), investigated CpG (genomic position GRCh37:10: 115612803, nt 1000 of SEQ ID
No. 34)
Seq ID no. 35, FLJ20581 - GRCh37:16: 20419653-20421653, Probe design region
(genomic position GRCh37:16: 20420592-20420713, nt 940 to nt 1061 of SEQ ID No.
35), investigated CpG (genomic position GRCh37:16: 20420653, nt 1000 of SEQ ID No.
35)
Seq ID no. 36, PTCD2 - GRCh37:5: 71613753-71615753, Probe design region (genomic
position GRCh37:5: 71614692-71614813, nt 940 to nt 1061 of SEQ ID No. 36),
investigated CpG (genomic position GRCh37:5: 71614753, nt 1000 of SEQ ID No. 36)
Seq ID no. 37, CX3CR1 - GRCh37:3: 39320450-39322450, Probe design region
(genomic position GRCh37:3: 39321389-39321510, nt 940 to nt 1061 of SEQ ID No.
37), investigated CpG (genomic position GRCh37:3: 39321450, nt 1000 of SEQ ID No.
37)
Seq ID no. 38, UBE2E1 - GRCh37:3: 23845693-23847693, Probe design region (genomic
position GRCh37:3: 23846632-23846753, nt 940 to nt 1061 of SEQ ID No. 38),
investigated CpG (genomic position GRCh37:3: 23846693, nt 1000 of SEQ ID No. 38)
Seq ID no. 39, IFT140 - GRCh37:16: 1656373-1658373, Probe design region (genomic
position GRCh37:16: 1657312-1657433, nt 940 to nt 1061 of SEQ ID No. 39),
investigated CpG (genomic position GRCh37:16: 1657373, nt 1000 of SEQ ID No. 39)
Seq ID no. 40, SLC22A8 - GRCh37:11: 62782123-62784123, Probe design region
(genomic position GRCh37:11: 62783063-62783184, nt 941 to nt 1062 of SEQ ID No.
40), investigated CpG (genomic position GRCh37:11: 62783123, nt 1001 of SEQ ID No.
40)
Seq ID no. 41, MLN - GRCh37:6: 33769851-33771851, Probe design region (genomic
position GRCh37:6: 33770790-33770911, nt 940 to nt 1061 of SEQ ID No. 41),
investigated CpG (genomic position GRCh37:6: 33770851, nt 1000 of SEQ ID No. 41)
Seq ID no. 42, DIRC1 - GRCh37:2: 189654590-189656590, Probe design region
(genomic position GRCh37:2: 189655529-189655650, nt 940 to nt 1061 of SEQ ID No.
42), investigated CpG (genomic position GRCh37:2: 189655590, nt 1000 of SEQ ID No.
42)
Seq ID no. 43, C1orf64 - GRCh37:1: 16329717-16331717, Probe design region (genomic
position GRCh37:1: 16330656-16330777, nt 940 to nt 1061 of SEQ ID No. 43),
investigated CpG (genomic position GRCh37:1: 16330717, nt 1000 of SEQ ID No. 43)
Seq ID no. 44, TNFSF13 - GRCh37:13: 108920053-108922053, Probe design region
(genomic position GRCh37:13: 108920992-108921113, nt 940 to nt 1061 of SEQ ID No.
44), investigated CpG (genomic position GRCh37:13: 108921053, nt 1000 of SEQ ID
No. 44)
Seq ID no. 45, CYP2B6 - GRCh37:19: 41495749-41497749, Probe design region
(genomic position GRCh37:19: 41496689-41496810, nt 941 to nt 1062 of SEQ ID No.
45), investigated CpG (genomic position GRCh37:19: 41496749, nt 1001 of SEQ ID No.
45)
Seq ID no. 46, ACSL5 - GRCh37:10: 114133551-114135551, Probe design region
(genomic position GRCh37:10: 114134490-114134611, nt 940 to nt 1061 of SEQ ID No.
46), investigated CpG (genomic position GRCh37:10: 114134551, nt 1000 of SEQ ID
No. 46)
Seq ID no. 47, SERPINB12 - GRCh37:18: 61221687-61223687, Probe design region
(genomic position GRCh37:18: 61222627-61222748, nt 941 to nt 1062 of SEQ ID No.
47), investigated CpG (genomic position GRCh37:18: 61222687, nt 1001 of SEQ ID No.
47)
Seq ID no. 48, APOBEC4 - GRCh37:1: 183621727-183623727, Probe design region
(genomic position GRCh37:1: 183622666-183622787, nt 940 to nt 1061 of SEQ ID No.
48), investigated CpG (genomic position GRCh37:1: 183622727, nt 1000 of SEQ ID No.
48)
Seq ID no. 49, TNNI1 - GRCh37:1: 201389794-201391794, Probe design region
(genomic position GRCh37:1: 201390734-201390855, nt 941 to nt 1062 of SEQ ID No.
49), investigated CpG (genomic position GRCh37:1: 201390794, nt 1001 of SEQ ID No.
49)
Seq ID no. 50, C1orf42 - GRCh37:1: 152486336-152488336, Probe design region
(genomic position GRCh37:1: 152487275-152487396, nt 940 to nt 1061 of SEQ ID No.
50), investigated CpG (genomic position GRCh37:1: 152487336, nt 1000 of SEQ ID No.
50)
Seq ID no. 51, PPAT - GRCh37:4: 57302150-57304150, Probe design region (genomic
position GRCh37:4: 57303089-57303210, nt 940 to nt 1061 of SEQ ID No. 51),
investigated CpG (genomic position GRCh37:4: 57303150, nt 1000 of SEQ ID No. 51)
Seq ID no. 52, PSD4 - GRCh37:2: 113930566-113932566, Probe design region (genomic
position GRCh37:2: 113931505-113931626, nt 940 to nt 1061 of SEQ ID No. 52),
investigated CpG (genomic position GRCh37:2: 113931566, nt 1000 of SEQ ID No. 52)
Seq ID no. 53, SCN4A - GRCh37:17: 62049038-62051038, Probe design region (genomic
position GRCh37:17: 62049978-62050099, nt 941 to nt 1062 of SEQ ID No. 53),
investigated CpG (genomic position GRCh37:17: 62050038, nt 1001 of SEQ ID No. 53)
Seq ID no. 54, IL8RB - GRCh37:2: 218989627-218991627, Probe design region
(genomic position GRCh37:2: 218990567-218990688, nt 941 to nt 1062 of SEQ ID No.
54), investigated CpG (genomic position GRCh37:2: 218990627, nt 1001 of SEQ ID No.
54)
Seq ID no. 55, UNC45B - GRCh37:17: 33473693-33475693, Probe design region
(genomic position GRCh37:17: 33474632-33474753, nt 940 to nt 1061 of SEQ ID No.
55), investigated CpG (genomic position GRCh37:17: 33474693, nt 1000 of SEQ ID No.
55)
Seq ID no. 56, C1orf201 - GRCh37:1: 24726851-24728851, Probe design region
(genomic position GRCh37:1: 24727790-24727911, nt 940 to nt 1061 of SEQ ID No. 56),
investigated CpG (genomic position GRCh37:1: 24727851, nt 1000 of SEQ ID No. 56)
Seq ID no. 57, SFTPG - GRCh37:6: 30899198-30901198, Probe design region (genomic
position GRCh37:6: 30900137-30900258, nt 940 to nt 1061 of SEQ ID No. 57),
investigated CpG (genomic position GRCh37:6: 30900198, nt 1000 of SEQ ID No. 57)
Seq ID no. 58, LOC124751 - GRCh37:17: 8274274-8276274, Probe design region
(genomic position GRCh37:17: 8275213-8275334, nt 940 to nt 1061 of SEQ ID No. 58),
investigated CpG (genomic position GRCh37:17: 8275274, nt 1000 of SEQ ID No. 58)
Seq ID no. 59, ANKRD1 - GRCh37:10: 92679511-92681511, Probe design region
(genomic position GRCh37:10: 92680450-92680571, nt 940 to nt 1061 of SEQ ID No.
59), investigated CpG (genomic position GRCh37:10: 92680511, nt 1000 of SEQ ID No.
59)
Seq ID no. 60, FMO2 - GRCh37:1: 171153612-171155612, Probe design region (genomic
position GRCh37:1: 171154552-171154673, nt 941 to nt 1062 of SEQ ID No. 60),
investigated CpG (genomic position GRCh37:1: 171154612, nt 1001 of SEQ ID No. 60)
Seq ID no. 61, ARSG - GRCh37:17: 66302328-66304328, Probe design region (genomic
position GRCh37:17: 66303268-66303389, nt 941 to nt 1062 of SEQ ID No. 61),
investigated CpG (genomic position GRCh37:17: 66303328, nt 1001 of SEQ ID No. 61)
Seq ID no. 62, GJB4 - GRCh37:1: 35225892-35227892, Probe design region (genomic
position GRCh37:1: 35226831-35226952, nt 940 to nt 1061 of SEQ ID No. 62),
investigated CpG (genomic position GRCh37:1: 35226892, nt 1000 of SEQ ID No. 62)
Seq ID no. 63, LRRN5 - GRCh37:1: 204654379-204656379, Probe design region
(genomic position GRCh37:1: 204655318-204655439, nt 940 to nt 1061 of SEQ ID No.
63), investigated CpG (genomic position GRCh37:1: 204655379, nt 1000 of SEQ ID No.
63)
Seq ID no. 64, MSMB - GRCh37:10: 51548260-51550260, Probe design region (genomic
position GRCh37:10: 51549200-51549321, nt 941 to nt 1062 of SEQ ID No. 64),
investigated CpG (genomic position GRCh37:10: 51549260, nt 1001 of SEQ ID No. 64)
Seq ID no. 65, HLA-DOB - GRCh37:6: 32784142-32786142, Probe design region
(genomic position GRCh37:6: 32785081-32785202, nt 940 to nt 1061 of SEQ ID No.
65), investigated CpG (genomic position GRCh37:6: 32785142, nt 1000 of SEQ ID No.
65)
Seq ID no. 66, R3HDML - GRCh37:20: 42963960-42965960, Probe design region
(genomic position GRCh37:20: 42964899-42965020, nt 940 to nt 1061 of SEQ ID No.
66), investigated CpG (genomic position GRCh37:20: 42964960, nt 1000 of SEQ ID No.
66)
Seq ID no. 67, SPINK5 - GRCh37:5: 147442252-147444252, Probe design region
(genomic position GRCh37:5: 147443192-147443313, nt 941 to nt 1062 of SEQ ID No.
67), investigated CpG (genomic position GRCh37:5: 147443252, nt 1001 of SEQ ID No.
67)
Seq ID no. 68, DLG5 - GRCh37:10: 79627941-79629941, Probe design region (genomic
position GRCh37:10: 79628881-79629002, nt 941 to nt 1062 of SEQ ID No. 68),
investigated CpG (genomic position GRCh37:10: 79628941, nt 1001 of SEQ ID No. 68)
Seq ID no. 69, PALLD - GRCh37:4: 169417492-169419492, Probe design region
(genomic position GRCh37:4: 169418431-169418552, nt 940 to nt 1061 of SEQ ID No.
69), investigated CpG (genomic position GRCh37:4: 169418492, nt 1000 of SEQ ID No.
69)
Seq ID no. 70, WFDC12 - GRCh37:20: 43752347-43754347, Probe design region
(genomic position GRCh37:20:43753287-43753408, nt 940 to nt 1061 of SEQ ID No.
70), investigated CpG (genomic position GRCh37:20: 43753347, nt 1000 of SEQ ID No.
70)
Seq ID no. 71, WDR20 - GRCh37:14: 102603722-102605722, Probe design region
(genomic position GRCh37:14: 102604662-102604783, nt 941 to nt 1062 of SEQ ID No.
71), investigated CpG (genomic position GRCh37:14: 102604722, nt 1001 of SEQ ID
No. 71)
Seq ID no. 72, FLJ32771 - GRCh37:11: 61247085-61249085, Probe design region
(genomic position GRCh37:11: 61248024-61248145, nt 940 to nt 1061 of SEQ ID No.
72), investigated CpG (genomic position GRCh37:11: 61248085, nt 1000 of SEQ ID No.
72)
Seq ID no. 73, PKHD1 - GRCh37:6: 51952114-51954114, Probe design region (genomic
position GRCh37:6: 51953054-51953175, nt 941 to nt 1062 of SEQ ID No. 73),
investigated CpG (genomic position GRCh37:6: 51953114, nt 1001 of SEQ ID No. 73)
Seq ID no. 74, DEFB123 - GRCh37:20: 30027893-30029893, Probe design region
(genomic position GRCh37:20:30028833-30028954, nt 941 to nt 1062 of SEQ ID No.
74), investigated CpG (genomic position GRCh37:20: 30028893, nt 1001 of SEQ ID No.
74)
Seq ID no. 75, KRTHB3 - GRCh37:12: 52713892-52715892, Probe design region
(genomic position GRCh37:12: 52714831-52714952, nt 940 to nt 1061 of SEQ ID No.
75), investigated CpG (genomic position GRCh37:12: 52714892, nt 1000 of SEQ ID No.
75)
Seq ID no. 76, BSND - GRCh37:1: 55463928-55465928, Probe design region (genomic
position GRCh37:1: 55464867-55464988, nt 940 to nt 1061 of SEQ ID No. 76),
investigated CpG (genomic position GRCh37:1: 55464928, nt 1000 of SEQ ID No. 76)
Seq ID no. 77, NALP10 - GRCh37:11: 7984184-7986184, Probe design region (genomic
position GRCh37:11: 7985123-7985244, nt 940 to nt 1061 of SEQ ID No. 77),
investigated CpG (genomic position GRCh37:11: 7985184, nt 1000 of SEQ ID No. 77)
Seq ID no. 78, NR5A2 - GRCh37:1: 199995238-199997238, Probe design region
(genomic position GRCh37:1: 7985123-7985244, nt 940 to nt 1061 of SEQ ID No. 78),
investigated CpG (genomic position GRCh37:1: 199996238, nt 1000 of SEQ ID No. 78)
Seq ID no. 79, KRT25D - GRCh37:17: 38955500-38957500, Probe design region
(genomic position GRCh37:17: 38956440-38956561, nt 941 to nt 1062 of SEQ ID No.
79), investigated CpG (genomic position GRCh37:17: 38956500, nt 1001 of SEQ ID No.
79)
Seq ID no. 80, APOD - GRCh37:3: 195310868-195312868, Probe design region (genomic
position GRCh37:3: 195311808-195311808, nt 941 to nt 1062 of SEQ ID No. 80),
investigated CpG (genomic position GRCh37:3: 195311868, nt 1001 of SEQ ID No. 80)
Seq ID no. 81, CLEC4F - GRCh37:2: 71046650-71048650, Probe design region
(genomic position GRCh37:2: 71047589-71047710, nt 940 to nt 1061 of SEQ ID No. 81),
investigated CpG (genomic position GRCh37:2: 71047650, nt 1000 of SEQ ID No. 81)
Seq ID no. 82, THRB - GRCh37:3: 24536178-24538178, Probe design region (genomic
position GRCh37:3: 24537117-24537238, nt 940 to nt 1061 of SEQ ID No. 82),
investigated CpG (genomic position GRCh37:3: 24537178, nt 1000 of SEQ ID No. 82)
Seq ID no. 83, KRTHB5 - GRCh37:12: 52760417-52762417, Probe design region
(genomic position GRCh37:12: 52761356-52761477, nt 940 to nt 1061 of SEQ ID No.
83), investigated CpG (genomic position GRCh37:12: 52761417, nt 1000 of SEQ ID No.
83)
Seq ID no. 84, SLC13A1 - GRCh37:7: 122838956-122840956, Probe design region
(genomic position GRCh37:7: 122839895-122840016, nt 940 to nt 1061 of SEQ ID No.
84), investigated CpG (genomic position GRCh37:7: 122839956, nt 1000 of SEQ ID No.
84)
Seq ID no. 85, C3orf27 - GRCh37:3: 128294141-128296141, Probe design region
(genomic position GRCh37:3: 128295081-128295202, nt 941 to nt 1062 of SEQ ID No.
85), investigated CpG (genomic position GRCh37:3: 128295141, nt 1001 of SEQ ID No.
85)
Seq ID no. 86, MGC35206 - GRCh37:22: 37403888-37405888, Probe design region
(genomic position GRCh37:22: 37404828-37404949, nt 941 to nt 1062 of SEQ ID No.
86), investigated CpG (genomic position GRCh37:22: 37404888, nt 1001 of SEQ ID No.
86)
Seq ID no. 87, C3orf63 - GRCh37:3: 56697168-56699168, Probe design region (genomic
position GRCh37:3: 56698108-56698229, nt 941 to nt 1062 of SEQ ID No. 87),
investigated CpG (genomic position GRCh37:3: 56698168, nt 1001 of SEQ ID No. 87)
Seq ID no. 88, DARC - GRCh37:1: 159173156-159175156, Probe design region
(genomic position GRCh37:1: 159174096-159174217, nt 941 to nt 1062 of SEQ ID No.
88), investigated CpG (genomic position GRCh37:1: 159174156, nt 1001 of SEQ ID No.
88)
Seq ID no. 89, SLC2A7 - GRCh37:1: 9085621-9087621, Probe design region (genomic
position GRCh37:1: 9086561-9086682, nt 941 to nt 1062 of SEQ ID No. 89),
investigated CpG (genomic position GRCh37:1: 9086621, nt 1001 of SEQ ID No. 89)
Seq ID no. 90, CPA3 - GRCh37:3: 148582153-148584153, Probe design region (genomic
position GRCh37:3: 148583092-148583213, nt 940 to nt 1061 of SEQ ID No. 90),
investigated CpG (genomic position GRCh37:3: 148583153, nt 1000 of SEQ ID No. 90)
Seq ID no. 91, MIS12 - GRCh37:17: 5390239-5392239, Probe design region (genomic
position GRCh37:17: 5391178-5391299, nt 940 to nt 1061 of SEQ ID No. 91),
investigated CpG (genomic position GRCh37:17: 5391239, nt 1000 of SEQ ID No. 91)
Seq ID no. 92, KRTHB4 - GRCh37:12: 52778282-52780282, Probe design region
(genomic position GRCh37:12: 52779221-52779342, nt 940 to nt 1061 of SEQ ID No.
92), investigated CpG (genomic position GRCh37:12: 52779282, nt 1000 of SEQ ID No.
92)
Seq ID no. 93, MRVI1 - GRCh37:11: 10714360-10716360, Probe design region (genomic
position GRCh37:11: 10715300-10715421, nt 941 to nt 1062 of SEQ ID No. 93),
investigated CpG (genomic position GRCh37:11: 10715360, nt 1001 of SEQ ID No. 93)
Seq ID no. 94, MIA2 - GRCh37:14: 39702303-39704303, Probe design region (genomic
position GRCh37:14: 39703242-39703363, nt 940 to nt 1061 of SEQ ID No. 94),
investigated CpG (genomic position GRCh37:14: 39703303, nt 1000 of SEQ ID No. 94)
Seq ID no. 95, CYP2A7 - GRCh37:19: 41388357-41390357, Probe design region
(genomic position GRCh37:19: 41389297-41389418, nt 941 to nt 1062 of SEQ ID No.
95), investigated CpG (genomic position GRCh37:19: 41389357, nt 1001 of SEQ ID No.
95)
Seq ID no. 96, ANGPT4 - GRCh37:20: 895349-897349, Probe design region (genomic
position GRCh37:20: 896289-896410, nt 941 to nt 1062 of SEQ ID No. 96), investigated
CpG (genomic position GRCh37:20: 896349, nt 1001 of SEQ ID No. 96)
Seq ID no. 97, SLC44A5 - GRCh37:1: 76077230-76079230, Probe design region
(genomic position GRCh37:1: 76078170-76078291, nt 941 to nt 1062 of SEQ ID No. 97),
investigated CpG (genomic position GRCh37:1: 76078230, nt 1001 of SEQ ID No. 97)
Seq ID no. 98, CST9L - GRCh37:20: 23548455-23550455, Probe design region (genomic
position GRCh37:20: 23549395-23549516, nt 941 to nt 1062 of SEQ ID No. 98),
investigated CpG (genomic position GRCh37:20: 23549455, nt 1001 of SEQ ID No. 98)
Seq ID no. 99, SLC18A2 - GRCh37:10: 118999236-119001236, Probe design region
(genomic position GRCh37:10: 119000176-119000297, nt 941 to nt 1062 of SEQ ID No.
99), investigated CpG (genomic position GRCh37:10: 119000236, nt 1001 of SEQ ID
No. 99)
Seq ID no. 100, SLC6A18 - GRCh37:5: 1224720-1226720, Probe design region (genomic
position GRCh37:5: 1225659-1225780, nt 940 to nt 1061 of SEQ ID No. 100),
investigated CpG (genomic position GRCh37:5: 1225720, nt 1000 of SEQ ID No. 100)
Seq ID no. 101, ALOX12B - GRCh37:17: 7989706-7991706, Probe design region
(genomic position GRCh37:17: 7990645-7990766, nt 940 to nt 1061 of SEQ ID No. 101),
investigated CpG (genomic position GRCh37:17: 7990706, nt 1000 of SEQ ID No. 101)
Seq ID no. 102, WNT10B - GRCh37:12: 49365538-49367538, Probe design region
(genomic position GRCh37:12: 49366478-49366599, nt 941 to nt 1062 of SEQ ID No.
102), investigated CpG (genomic position GRCh37:12: 49366538, nt 1001 of SEQ ID
No. 102)
Seq ID no. 103, S100A9 - GRCh37:1: 153328013-153330013, Probe design region
(genomic position GRCh37:1: 153328953-153329074, nt 941 to nt 1062 of SEQ ID No.
103), investigated CpG (genomic position GRCh37:1: 153329013, nt 1001 of SEQ ID
No. 103)
Seq ID no. 104, SLC6A11 - GRCh37:3: 10856457-10858457, Probe design region
(genomic position GRCh37:3: 10857396-10857517, nt 940 to nt 1061 of SEQ ID No.
104), investigated CpG (genomic position GRCh37:3: 10857457, nt 1000 of SEQ ID No.
104)
Seq ID no. 105, FGF4 - GRCh37:11: 69590383-69592383, Probe design region (genomic
position GRCh37:11: 69591323-69591444, nt 941 to nt 1062 of SEQ ID No. 105),
investigated CpG (genomic position GRCh37:11: 69591383, nt 1001 of SEQ ID No. 105)
Seq ID no. 106, PCDHAC2 - GRCh37:5: 140345922-140347922, Probe design region
(genomic position GRCh37:5: 140346861-140346982, nt 940 to nt 1061 of SEQ ID No.
106), investigated CpG (genomic position GRCh37:5: 140346922 nt 1000 of SEQ ID No.
106)
Seq ID no. 107, GRM4 - GRCh37:6: 34100527-34102527, Probe design region (genomic
position GRCh37:6: 34101466-34101587, nt 940 to nt 1061 of SEQ ID No. 107),
investigated CpG (genomic position GRCh37:6: 34101527, nt 1000 of SEQ ID No. 107)
Seq ID no. 108, ANXA13 - GRCh37:8: 124748565-124750565, Probe design region
(genomic position GRCh37:8: 124749504-124749625, nt 940 to nt 1061 of SEQ ID No.
108), investigated CpG (genomic position GRCh37:8: 124749565, nt 1000 of SEQ ID
No. 108)
Seq ID no. 109, CTAG2 - GRCh37:X: 153880680-153882680, Probe design region
(genomic position GRCh37:X: 153880619-153880740, nt 940 to nt 1061 of SEQ ID No.
109), investigated CpG (genomic position GRCh37:X: 153880680, nt 1000 of SEQ ID
No. 109)
Seq ID no. 110, PVALB - GRCh37:22: 37215968-37217068, Probe design region
(genomic position GRCh37:22: 37216007-37216128, nt 940 to nt 1061 of SEQ ID No.
110), investigated CpG (genomic position GRCh37:22: 37216068, nt 1000 of SEQ ID
No. 110)
Seq ID no. 111, HAO2 - GRCh37:1: 119910481-119912481, Probe design region
(genomic position GRCh37:1: 119911420-119911541, nt 940 to nt 1061 of SEQ ID No.
111), investigated CpG (genomic position GRCh37:1: 119911481, nt 1000 of SEQ ID
No. 111)
Seq ID no. 112, SLC4A7 - GRCh37:3: 27497660-27499660, Probe design region
(genomic position GRCh37:3: 27498599-27498720, nt 940 to nt 1061 of SEQ ID No.
112), investigated CpG (genomic position GRCh37:3: 27498660, nt 1000 of SEQ ID No.
112)
Seq ID no. 113, TDRD5 - GRCh37:1: 179560500-179562500, Probe design region
(genomic position GRCh37:1: 179561440-179561561, nt 941 to nt 1062 of SEQ ID No.
113), investigated CpG (genomic position GRCh37:1: 179561500, nt 1001 of SEQ ID
No. 113)
Seq ID no. 114, FAAH - GRCh37:1: 46858774-46860774, Probe design region (genomic
position GRCh37:1: 46859714-46859835, nt 941 to nt 1062 of SEQ ID No. 114),
investigated CpG (genomic position GRCh37:1: 46859774, nt 1001 of SEQ ID No. 114)
Seq ID no. 115, CD1D - GRCh37:1: 158148228-158150228, Probe design region
(genomic position GRCh37:1: 158149168-158149289, nt 941 to nt 1062 of SEQ ID No.
115), investigated CpG (genomic position GRCh37:1: 158149228, nt 1001 of SEQ ID
No. 115)
Seq ID no. 116, FLJ39501 - GRCh37:19: 15617560-15619560, Probe design region
(genomic position GRCh37:19: 15618499-15618620, nt 940 to nt 1061 of SEQ ID No.
116), investigated CpG (genomic position GRCh37:19: 15618560, nt 1000 of SEQ ID
No. 116)
Seq ID no. 117, AGLT1D1 - GRCh37:12: 129336768-129338768, Probe design region
(genomic position GRCh37:12: 129337708-129337829, nt 941 to nt 1062 of SEQ ID No.
117), investigated CpG (genomic position GRCh37:12: 129337768, nt 1001 of SEQ ID
No. 117)
Seq ID no. 118, IL28B - GRCh37:19: 39734568-39736568, Probe design region (genomic
position GRCh37:19: 39735508-39735629, nt 941 to nt 1062 of SEQ ID No. 118),
investigated CpG (genomic position GRCh37:19: 39735568, nt 1001 of SEQ ID No. 118)
Seq ID no. 119, AVPR1B - GRCh37:1: 206222242-206224242, Probe design region
(genomic position GRCh37:1: 206223181-206223302, nt 940 to nt 1061 of SEQ ID No.
119), investigated CpG (genomic position GRCh37:1: 206223242, nt 1000 of SEQ ID
No. 119)
Seq ID no. 120, GJB3 - GRCh37:1: 35245607-35247607, Probe design region (genomic
position GRCh37:1: 35246547-35246668, nt 941 to nt 1062 of SEQ ID No. 120),
investigated CpG (genomic position GRCh37:1: 35246607, nt 1001 of SEQ ID No. 120)
Seq ID no. 121, GABRG3 - GRCh37:15: 27669336-27671336, Probe design region
(genomic position GRCh37:15: 27670275-27670396, nt 940 to nt 1061 of SEQ ID No. 121),
investigated CpG (genomic position GRCh37:15: 27670336, nt 1000 of SEQ ID No. 121)
Seq ID no. 122, CTAG1B - GRCh37:X: 153812580-153814580, Probe design region
(genomic position GRCh37:X: 153812520-153812641, nt 941 to nt 1062 of SEQ ID No. 122),
investigated CpG (genomic position GRCh37:X: 153812580, nt 1001 of SEQ ID No. 122)
Seq ID no. 123, UNQ9433 - GRCh37:8: 53477706-53479706, Probe design region (genomic
position GRCh37:8: 53478646-53478767, nt 941 to nt 1062 of SEQ ID No. 123), investigated
CpG (genomic position GRCh37:8: 53478706, nt 1001 of SEQ ID No. 123)
Seq ID no. 124, CEACAM6 - GRCh37:19: 42259486-42261486, Probe design region
(genomic position GRCh37:19: 42260425-42260546, nt 940 to nt 1061 of SEQ ID No. 124),
investigated CpG (genomic position GRCh37:19: 42260486, nt 1000 of SEQ ID No. 124)
Seq ID no. 125, DENND2D - GRCh37:1: 111742369-111744369, Probe design region
(genomic position GRCh37:1: 111743308-111743429, nt 940 to nt 1061 of SEQ ID No. 125),
investigated CpG (genomic position GRCh37:1: 111743369, nt 1000 of SEQ ID No. 125)
Seq ID no. 126, PGCP - GRCh37:8: 97656073-97658073, Probe design region (genomic
position GRCh37:8: 97657012-97657133, nt 940 to nt 1061 of SEQ ID No. 126), investigated
CpG (genomic position GRCh37:8: 97657073, nt 1000 of SEQ ID No. 126)
Seq ID no. 127, KCNG1 - GRCh37:20: 49637967-49639967, Probe design region (genomic
position GRCh37:20: 49638907-49639028, nt 941 to nt 1062 of SEQ ID No. 127),
investigated CpG (genomic position GRCh37:20: 49638967, nt 1001 of SEQ ID No. 127)
Seq ID no. 128, FAM107B - GRCh37:10: 14815654-14817654, Probe design region
(genomic position GRCh37:10: 14816594-14816715, nt 941 to nt 1062 of SEQ ID No. 128),
investigated CpG (genomic position GRCh37:10: 14816654, nt 1001 of SEQ ID No. 128)
Seq ID no. 129, FANCE - GRCh37:6: 35419754-35421754, Probe design region (genomic
position GRCh37:6: 35420694-35420815, nt 941 to nt 1062 of SEQ ID No. 129), investigated
CpG (genomic position GRCh37:6: 35420754, nt 1001 of SEQ ID No. 129)
Seq ID no. 130, FAM38B - GRCh37:18: 10696032-10698032, Probe design region (genomic
position GRCh37:18: 10696972-10697093, nt 941 to nt 1062 of SEQ ID No. 130),
investigated CpG (genomic position GRCh37:18: 10697032, nt 1001 of SEQ ID No. 130)
Seq ID no. 131, FOXF2 - GRCh37:6: 1388146-1390146, Probe design region (genomic
position GRCh37:6: 1389086-1389207, nt 941 to nt 1062 of SEQ ID No. 131), investigated
CpG (genomic position GRCh37:6: 1389146, nt 1001 of SEQ ID No. 131)
Seq ID no. 132, BGN - GRCh37:X: 152759079-152761079, Probe design region (genomic
position GRCh37:X: 152760018-152760139, nt 940 to nt 1061 of SEQ ID No. 132),
investigated CpG (genomic position GRCh37:X: 152760079, nt 1000 of SEQ ID No. 132)
Seq ID no. 133, MAGEA10 - GRCh37:X: 151305719-151307719, Probe design region
(genomic position GRCh37:X: 151306658-151306779, nt 940 to nt 1061 of SEQ ID No. 133),
investigated CpG (genomic position GRCh37:X: 151306719, nt 1000 of SEQ ID No. 133)
Seq ID no. 134, SPATS1 - GRCh37:6: 44309640-44311640, Probe design region (genomic
position GRCh37:6: 44310580-44310701, nt 941 to nt 1062 of SEQ ID No. 134), investigated
CpG (genomic position GRCh37:6: 44310640, nt 1001 of SEQ ID No. 134)
Seq ID no. 135, PRXX1 - GRCh37:1: 170631093-170633093, Probe design region (genomic
position GRCh37:1: 170632032-170632153, nt 940 to nt 1061 of SEQ ID No. 135),
investigated CpG (genomic position GRCh37:1: 170632093, nt 1000 of SEQ ID No. 135)
Seq ID no. 136, MLSTD1 - GRCh37:12: 29375483-29377483, Probe design region
(genomic position GRCh37:12: 29376423-29376544, nt 941 to nt 1062 of SEQ ID No. 136),
investigated CpG (genomic position GRCh37:12: 29376483, nt 1001 of SEQ ID No. 136)
Seq ID no. 137, PDZD4 - GRCh37:X: 153095786-153097786, Probe design region (genomic
position GRCh37:X: 153096726-153096847, nt 941 to nt 1062 of SEQ ID No. 137),
investigated CpG (genomic position GRCh37:X: 153096786, nt 1001 of SEQ ID No. 137)
Seq ID no. 138, HCP5 - GRCh37:6: 31430101-31432101, Probe design region (genomic
position GRCh37:6: 31431040-31431161, nt 940 to nt 1061 of SEQ ID No. 138), investigated
CpG (genomic position GRCh37:6: 31431101, nt 1000 of SEQ ID No. 138)
Seq ID no. 139, OCIAD2 - GRCh37:4: 48907902-48909902, Probe design region (genomic
position GRCh37:4: 48908842-48908963, nt 941 to nt 1062 of SEQ ID No. 139), investigated
CpG (genomic position GRCh37:4: 48908902, nt 1001 of SEQ ID No. 139)
Seq ID no. 140, CSAG1 - GRCh37:X: 151882197-151884197, Probe design region (genomic
position GRCh37:X: 151883137-153096258, nt 940 to nt 1061 of SEQ ID No. 140),
investigated CpG (genomic position GRCh37:X: 151883197, nt 1000 of SEQ ID No. 140)
Seq ID no. 141, SLCO4A1 - GRCh37:20: 61272056-61274056, Probe design region
(genomic position GRCh37:20: 61272995-61273116, nt 940 to nt 1061 of SEQ ID No. 141),
investigated CpG (genomic position GRCh37:20: 61273056, nt 1000 of SEQ ID No. 141)
Seq ID no. 142, C9orf90 - GRCh37:9: 130827969-130829969, Probe design region
(genomic position GRCh37:9: 130828909-130829030, nt 941 to nt 1062 of SEQ ID No. 142),
investigated CpG (genomic position GRCh37:9: 130828969, nt 1001 of SEQ ID No. 142)
Seq ID no. 143, CSAG1 - GRCh37:X: 151902447-151904447, Probe design region (genomic
position GRCh37:X: 151903387-151903508, nt 941 to nt 1062 of SEQ ID No. 143),
investigated CpG (genomic position GRCh37:X: 151903447, nt 1001 of SEQ ID No. 143)
Seq ID no. 144, AMPH - GRCh37:7: 38670659-38672659, Probe design region (genomic
position GRCh37:7: 38671599-38671720, nt 941 to nt 1062 of SEQ ID No. 144), investigated
CpG (genomic position GRCh37:7: 38671659, nt 1001 of SEQ ID No. 144)
Seq ID no. 145, MT1G - GRCh37:16: 56701134-56703134, Probe design region (genomic
position GRCh37:16: 56702074-56702195, nt 941 to nt 1062 of SEQ ID No. 145),
investigated CpG (genomic position GRCh37:16: 56702134, nt 1001 of SEQ ID No. 145)
Seq ID no. 146, C20orf100 - GRCh37:20: 42543648-42545648, Probe design region
(genomic position GRCh37:20: 42544588-42544709, nt 941 to nt 1062 of SEQ ID No. 146),
investigated CpG (genomic position GRCh37:20: 42544648, nt 1001 of SEQ ID No. 146)
Seq ID no. 147, CST7 - GRCh37:20: 24928716-24930716, Probe design region (genomic
position GRCh37:20: 24929656-24929777, nt 941 to nt 1062 of SEQ ID No. 147),
investigated CpG (genomic position GRCh37:17: 24929716, nt 1001 of SEQ ID No. 147)
Seq ID no. 148, KIAA0367 - GRCh37:9: 79327644-79329644, Probe design region
(genomic position GRCh37:9: 79328584-79328705, nt 941 to nt 1062 of SEQ ID No. 148),
investigated CpG (genomic position GRCh37:9: 79328644, nt 1001 of SEQ ID No. 148)
Seq ID no. 149, RNASE1 - GRCh37:14: 21270313-21272313, Probe design region (genomic
position GRCh37:14: 21271253-21271374, nt 941 to nt 1062 of SEQ ID No. 149),
investigated CpG (genomic position GRCh37:14: 21271313, nt 1001 of SEQ ID No. 149)
Seq ID no. 150, HCP5 - GRCh37:6: 31429916-31431916, Probe design region (genomic
position GRCh37:6: 31430856-31430974, nt 941 to nt 1062 of SEQ ID No. 150), investigated
CpG (genomic position GRCh37:6: 31430916, nt 1001 of SEQ ID No. 150)
Seq ID no. 151, NDN - GRCh37:15: 23931374:23933374, Probe design region (genomic
position GRCh37:15: 23932313-23932434, nt 940 to nt 1061 of SEQ ID No. 151),
investigated CpG (genomic position GRCh37:15: 23932374, nt 1000 of SEQ ID No. 151)
Seq ID no. 152, RAC2 - GRCh37:22: 37639403-37641403, Probe design region (genomic
position GRCh37:22: 37640342-37640463, nt 940 to nt 1061 of SEQ ID No. 152),
investigated CpG (genomic position GRCh37:22: 37640403, nt 1000 of SEQ ID No. 152)
Seq ID no. 153, AXL - GRCh37:19: 41724333-41726333, Probe design region (genomic
position GRCh37:19: 41725272-41725393, nt 940 to nt 1061 of SEQ ID No. 153),
investigated CpG (genomic position GRCh37:19: 41725333, nt 1000 of SEQ ID No. 153)
Seq ID no. 154, BATF - GRCh37:14: 75987747-75989747, Probe design region (genomic
position GRCh37:14: 75988687-75988808, nt 941 to nt 1062 of SEQ ID No. 154),
investigated CpG (genomic position GRCh37:14: 75988747, nt 1001 of SEQ ID No. 154)
Seq ID no. 155, EMILIN1 - GRCh37:2: 27300490-27302490, Probe design region (genomic
position GRCh37:2: 27301430-27301551, nt 941 to nt 1062 of SEQ ID No. 155), investigated
CpG (genomic position GRCh37:2: 27301490, nt 1001 of SEQ ID No. 155)
Seq ID no. 156, ARHGEF7 - GRCh37:13: 111765903:111767903, Probe design region
(genomic position GRCh37:13: 111766842-111766963, nt 940 to nt 1061 of SEQ ID No.
156), investigated CpG (genomic position GRCh37:13: 111766903, nt 1000 of SEQ ID No.
156)
Seq ID no. 157, EVL - GRCh37:14: 100531037-100533037, Probe design region (genomic
position GRCh37:14: 100531976-100532097, nt 940 to nt 1061 of SEQ ID No. 157),
investigated CpG (genomic position GRCh37:14: 100532037, nt 1000 of SEQ ID No. 157)
Seq ID no. 158, RAC2 - GRCh37:22: 37639251-37641251, Probe design region (genomic
position GRCh37:22: 37640190-37640311, nt 940 to nt 1061 of SEQ ID No. 158),
investigated CpG (genomic position GRCh37:22: 37640251, nt 1000 of SEQ ID No. 158)
Seq ID no. 159, KIAA0367 - GRCh37:9: 79327522-79329522, Probe design region
(genomic position GRCh37:9: 79328462-79328583, nt 941 to nt 1062 of SEQ ID No. 159),
investigated CpG (genomic position GRCh37:9: 79328522, nt 1001 of SEQ ID No. 159)
Seq ID no. 160, TFF3 - GRCh37:21: 43735927-43737927, Probe design region (genomic
position GRCh37:21: 43736866-43736987, nt 940 to nt 1061 of SEQ ID No. 160),
investigated CpG (genomic position GRCh37:21: 43736927, nt 1000 of SEQ ID No. 160)
Seq ID no. 161, CTSK - GRCh37:1: 150779684-150781684, Probe design region (genomic
position GRCh37:1: 150780624-150780745, nt 941 to nt 1062 of SEQ ID No. 161),
investigated CpG (genomic position GRCh37:1: 150780684, nt 1001 of SEQ ID No. 161)
Seq ID no. 162, C21orf70 - GRCh37:21: 46357703-46359703, Probe design region
(genomic position GRCh37:21: 46358642-46358763, nt 940 to nt 1061 of SEQ ID No. 162),
investigated CpG (genomic position GRCh37:21: 46358703, nt 1000 of SEQ ID No. 162)
Seq ID no. 163, PRKCZ - GRCh37:1: 1980271-1982271, Probe design region (genomic
position GRCh37:1: 1981210-1981331, nt 940 to nt 1061 of SEQ ID No. 163), investigated
CpG (genomic position GRCh37:1: 1981271, nt 1000 of SEQ ID No. 163)
Seq ID no. 164, MLSTD1 - GRCh37:12: 29375873-29377873, Probe design region
(genomic position GRCh37:12: 29376812-29376933, nt 940 to nt 1061 of SEQ ID No. 164),
investigated CpG (genomic position GRCh37:12: 29376873, nt 1000 of SEQ ID No. 164)
Seq ID no. 165, COL5A2 - GRCh37:2: 190043636-190045636, Probe design region
(genomic position GRCh37:2: 190044576-190044697, nt 941 to nt 1062 of SEQ ID No. 165),
investigated CpG (genomic position GRCh37:2: 190044636, nt 1001 of SEQ ID No. 165)
Seq ID no. 166, LY96 - GRCh37:8: 74902801-74904801, Probe design region (genomic
position GRCh37:8: 74903741-74903862, nt 941 to nt 1062 of SEQ ID No. 166), investigated
CpG (genomic position GRCh37:8: 74903801, nt 1001 of SEQ ID No. 166)
Seq ID no. 167, MAGE1 - GRCh37:X: 152484938-152486938, Probe design region
(genomic position GRCh37:X: 152485878-152485999, nt 941 to nt 1062 of SEQ ID No. 167),
investigated CpG (genomic position GRCh37:X: 152485938, nt 1001 of SEQ ID No. 167)
Seq ID no. 168, MAGEC2 - GRCh37:X: 141292020-141294020, Probe design region
(genomic position GRCh37:X: 141292960-141293081, nt 941 to nt 1062 of SEQ ID No. 168),
investigated CpG (genomic position GRCh37:X: 141293020, nt 1001 of SEQ ID No. 168)
Seq ID no. 169, DENND2D - GRCh37:1: 111742271-111744271, Probe design region
(genomic position GRCh37:1: 111743211-111743332, nt 941 to nt 1062 of SEQ ID No. 169),
investigated CpG (genomic position GRCh37:1: 111743271, nt 1001 of SEQ ID No. 169)
Seq ID no. 170, PPIE - GRCh37:1: 40203286-40205286, Probe design region (genomic
position GRCh37:1: 40204225-40204346, nt 940 to nt 1061 of SEQ ID No. 170), investigated
CpG (genomic position GRCh37:1: 40204286, nt 1000 of SEQ ID No. 170)
Seq ID no. 171, SORBS2 - GRCh37:4: 186876779-186878779, Probe design region
(genomic position GRCh37:4: 186877718-186877839, nt 940 to nt 1061 of SEQ ID No. 171),
investigated CpG (genomic position GRCh37:4: 186877779, nt 1000 of SEQ ID No. 171)
Seq ID no. 172, PROCR - GRCh37:20: 33759017-33761017, Probe design region (genomic
position GRCh37:20: 33759957-33760078, nt 941 to nt 1062 of SEQ ID No. 172),
investigated CpG (genomic position GRCh37:20: 33760017, nt 1001 of SEQ ID No. 172)
Seq ID no. 173, TMEPAI - GRCh37:20: 56285392-56287392, Probe design region (genomic
position GRCh37:20: 56286331-56286452, nt 940 to nt 1061 of SEQ ID No. 173),
investigated CpG (genomic position GRCh37:20: 56286392, nt 1000 of SEQ ID No. 173)
Seq ID no. 174, SSNA1 - GRCh37:9: 140080916-140082916, Probe design region (genomic
position GRCh37:9: 140081856-140081977, nt 941 to nt 1062 of SEQ ID No. 174),
investigated CpG (genomic position GRCh37:9: 140081916, nt 1001 of SEQ ID No. 174)
Seq ID no. 175, MGMT - GRCh37:10: 131263840-131265840, Probe design region
(genomic position GRCh37:10: 131264780-131264901, nt 941 to nt 1062 of SEQ ID No.
175), investigated CpG (genomic position GRCh37:10: 131264840, nt 1001 of SEQ ID No.
175)
Seq ID no. 176, GAGE2 - GRCh37:X: 49225695-49227695, Probe design region (genomic
position GRCh37:X: 49226635-49226756, nt 941 to nt 1062 of SEQ ID No. 176),
investigated CpG (genomic position GRCh37:X: 49226695, nt 1001 of SEQ ID No. 176)
Seq ID no. 177, C11orf1 - GRCh37:11: 111749355-111751355, Probe design region
(genomic position GRCh37:11: 111750295-111750416, nt 941 to nt 1062 of SEQ ID No.
177), investigated CpG (genomic position GRCh37:11: 111750355, nt 1001 of SEQ ID No.
177)
Seq ID no. 178, ABCB4 - GRCh37:7: 87108371-87110371, Probe design region (genomic
position GRCh37:7: 87109310-87109431, nt 940 to nt 1061 of SEQ ID No. 178), investigated
CpG (genomic position GRCh37:7: 87109371, nt 1000 of SEQ ID No. 178)
Seq ID no. 179, CTNNAL1 - GRCh37:9: 111775779-111777779, Probe design region
(genomic position GRCh37:9: 111776719-111776840, nt 941 to nt 1062 of SEQ ID No. 179),
investigated CpG (genomic position GRCh37:9: 111776779, nt 1001 of SEQ ID No. 179)
Seq ID no. 180, EMP1 - GRCh37:12: 13347429-13349429, Probe design region (genomic
position GRCh37:12: 13348369-13348490, nt 941 to nt 1062 of SEQ ID No. 180),
investigated CpG (genomic position GRCh37:12: 13348429, nt 1001 of SEQ ID No. 180)
Seq ID no. 181, RNASE1 - GRCh37:14: 21269924-21271924, Probe design region (genomic
position GRCh37:14: 21270864-21270985, nt 941 to nt 1062 of SEQ ID No. 181),
investigated CpG (genomic position GRCh37:14: 21270924, nt 1001 of SEQ ID No. 181)
Seq ID no. 182, MAGEA6 - GRCh37:X: 151866333-151868333, Probe design region
(genomic position GRCh37:X: 151867273-151867394, nt 941 to nt 1062 of SEQ ID No. 182),
investigated CpG (genomic position GRCh37:X: 151867333, nt 1001 of SEQ ID No. 182)
Seq ID no. 183, CHMP4A - GRCh37:14: 24682529-24684529, Probe design region
(genomic position GRCh37:14: 24683468-24683589, nt 940 to nt 1061 of SEQ ID No. 183),
investigated CpG (genomic position GRCh37:14: 24683529, nt 1000 of SEQ ID No. 183)
Seq ID no. 184, ADORA2B - GRCh37:17: 15846413-15848413, Probe design region
(genomic position GRCh37:17: 15847352-15847473, nt 940 to nt 1061 of SEQ ID No. 184),
investigated CpG (genomic position GRCh37:17: 15847413, nt 1000 of SEQ ID No. 184)
Seq ID no. 185, PLA1A - GRCh37:3: 119309382-119311382, Probe design region (genomic
position GRCh37:3: 119310321-119310442, nt 940 to nt 1061 of SEQ ID No. 185),
investigated CpG (genomic position GRCh37:3: 119310382, nt 1000 of SEQ ID No. 185)
Seq ID no. 186, KIAA0141 - GRCh37:5: 141301730-141303730, Probe design region
(genomic position GRCh37:3: 141302669-141302790, nt 940 to nt 1061 of SEQ ID No. 186),
investigated CpG (genomic position GRCh37:3: 141302730, nt 1000 of SEQ ID No. 186)
Seq ID no. 187, OLFM1 - GRCh37:9: 137965585-137967585, Probe design region (genomic
position GRCh37:9: 137966524-137966645, nt 940 to nt 1061 of SEQ ID No. 187),
investigated CpG (genomic position GRCh37:9: 137966585, nt 1000 of SEQ ID No. 187)
Seq ID no. 188, ERO1L - GRCh37:14: 53162205-53164205, Probe design region (genomic
position GRCh37:14: 53163144-53163265, nt 940 to nt 1061 of SEQ ID No. 188),
investigated CpG (genomic position GRCh37:14: 53163205, nt 1000 of SEQ ID No. 188)
Seq ID no. 189, CXorf12 - GRCh37:X: 153237580-153239580, Probe design region
(genomic position GRCh37:X: 153238519-153238640, nt 940 to nt 1061 of SEQ ID No. 189),
investigated CpG (genomic position GRCh37:X: 153238580, nt 1000 of SEQ ID No. 189)
Seq ID no. 190, ARHGDIB - GRCh37:12: 15113704-15115704, Probe design region
(genomic position GRCh37:12: 15114643-15114764, nt 940 to nt 1061 of SEQ ID No. 190),
investigated CpG (genomic position GRCh37:12: 15114704, nt 1000 of SEQ ID No. 190)
Seq ID no. 191, GRAMD3 - GRCh37:5: 125758291-125760291, Probe design region
(genomic position GRCh37:5: 125759231-125759352, nt 941 to nt 1062 of SEQ ID No. 191),
investigated CpG (genomic position GRCh37:5: 125759291, nt 1001 of SEQ ID No. 191)
Seq ID no. 192, PLD1 - GRCh37:3: 171454827-171456827, Probe design region (genomic
position GRCh37:3: 171455766-171455887, nt 940 to nt 1061 of SEQ ID No. 192),
investigated CpG (genomic position GRCh37:3: 171455827, nt 1000 of SEQ ID No. 192)
Seq ID no. 193, HNMT - GRCh37:2: 138720318-138722318, Probe design region (genomic
position GRCh37:2: 138721257-138721378, nt 940 to nt 1061 of SEQ ID No. 193),
investigated CpG (genomic position GRCh37:2: 138721318, nt 1000 of SEQ ID No. 193)
Seq ID no. 194, ZNF558 - GRCh37:19: 8933543-8935543, Probe design region (genomic
position GRCh37:19: 8934483-8934604, nt 941 to nt 1062 of SEQ ID No. 194), investigated
CpG (genomic position GRCh37:19: 8934543, nt 1001 of SEQ ID No. 194)
Seq ID no. 195, ATP6V1E2 - GRCh37:2: 46745935-46747935, Probe design region
(genomic position GRCh37:2: 46746874-46746995, nt 940 to nt 1061 of SEQ ID No. 195),
investigated CpG (genomic position GRCh37:2: 46746935, nt 1000 of SEQ ID No. 195)
Seq ID no. 196, KRT25C - GRCh37:17: 38939133-38941133, Probe design region (genomic
position GRCh37:17: 38940072-38940193, nt 940 to nt 1061 of SEQ ID No. 196),
investigated CpG (genomic position GRCh37:17: 38940133, nt 1000 of SEQ ID No. 196)
Seq ID no. 197, ZNF532 - GRCh37:18: 56528784-56530784, Probe design region (genomic
position GRCh37:18: 56529724-56529845, nt 941 to nt 1062 of SEQ ID No. 197),
investigated CpG (genomic position GRCh37:18: 56529784, nt 1001 of SEQ ID No. 197)
Seq ID no. 198, SLAMF9 - GRCh37:1: 159924416-159926416, Probe design region
(genomic position GRCh37:1: 159925356-159925477, nt 941 to nt 1062 of SEQ ID No. 198),
investigated CpG (genomic position GRCh37:1: 159925416, nt 1001 of SEQ ID No. 198)
Seq ID no. 199, NBL1 - GRCh37:1: 19967885-19969885, Probe design region (genomic
position GRCh37:1: 19968825-19968946, nt 941 to nt 1062 of SEQ ID No. 199), investigated
CpG (genomic position GRCh37:1: 19968885, nt 1001 of SEQ ID No. 199)
Seq ID no. 200, MAGEA10 - GRCh37:X: 151306054-151308054, Probe design region
(genomic position GRCh37:X: 151306993-151307114, nt 940 to nt 1061 of SEQ ID No. 200),
investigated CpG (genomic position GRCh37:X: 151307054, nt 1000 of SEQ ID No. 200)
Seq ID no. 201, ABCA8 - GRCh37:17: 66949546-66951546, Probe design region (genomic
position GRCh37:17: 66950485-66950606, nt 940 to nt 1061 of SEQ ID No. 201),
investigated CpG (genomic position GRCh37:17: 66950546, nt 1000 of SEQ ID No. 201)
Seq ID no. 202, ACOX2 - GRCh37:3: 58521690-58523690, Probe design region (genomic
position GRCh37:3: 58522629-58522750, nt 940 to nt 1061 of SEQ ID No. 202), investigated
CpG (genomic position GRCh37:3: 58522690, nt 1000 of SEQ ID No. 202)
Seq ID no. 203, TIMP3 - GRCh37:22: 33196035-33198035, Probe design region (genomic
position GRCh37:22: 33196974-33197095, nt 940 to nt 1061 of SEQ ID No. 203),
investigated CpG (genomic position GRCh37:22: 33197035, nt 1000 of SEQ ID No. 203)
Seq ID no. 204, RPUSD3 - GRCh37:3: 9885233-9887233, Probe design region (genomic
position GRCh37:3: 9886172-9886293, nt 940 to nt 1061 of SEQ ID No. 204), investigated
CpG (genomic position GRCh37:3: 9886233, nt 1000 of SEQ ID No. 204)
Seq ID no. 205, PRDX2 - GRCh37:19: 12911528-12913528, Probe design region (genomic
position GRCh37:19: 12912467-12912588, nt 940 to nt 1061 of SEQ ID No. 205),
investigated CpG (genomic position GRCh37:19: 12912528, nt 1000 of SEQ ID No. 205)
Seq ID no. 206, PCOLCE - GRCh37:7: 100199010-100201010, Probe design region
(genomic position GRCh37:7: 100199949-100200070, nt 940 to nt 1061 of SEQ ID No. 206),
investigated CpG (genomic position GRCh37:7: 100200010, nt 1000 of SEQ ID No. 206)
Seq ID no. 207, OCIAD2 - GRCh37:4: 48907469-48909469, Probe design region (genomic
position GRCh37:4: 48908409-48908530, nt 941 to nt 1062 of SEQ ID No. 207), investigated
CpG (genomic position GRCh37:4: 48908469, nt 1001 of SEQ ID No. 207)
Seq ID no. 208, CTSK - GRCh37:1: 150780435-150782435, Probe design region (genomic
position GRCh37:1: 150781374-150781495, nt 940 to nt 1061 of SEQ ID No. 208),
investigated CpG (genomic position GRCh37:1: 150781435, nt 1000 of SEQ ID No. 208)
Seq ID no. 209, ADM - GRCh37:11: 10325924-10327924, Probe design region (genomic
position GRCh37:11: 10326863-10326984, nt 940 to nt 1061 of SEQ ID No. 209),
investigated CpG (genomic position GRCh37:11: 10326924, nt 1000 of SEQ ID No. 209)
Seq ID no. 210, PAGE5 - GRCh37:X: 55245456-55247456, Probe design region (genomic
position GRCh37:X: 55246395-55246516, nt 940 to nt 1061 of SEQ ID No. 210),
investigated CpG (genomic position GRCh37:X: 55246456, nt 1000 of SEQ ID No. 210)
Seq ID no. 211, NNMT - GRCh37:11: 114165636-114167636, Probe design region (genomic
position GRCh37:11: 114166576-114166697, nt 941 to nt 1062 of SEQ ID No. 211),
investigated CpG (genomic position GRCh37:11: 114166636, nt 1001 of SEQ ID No. 211)
Seq ID no. 212, MAGEA3 - GRCh37:X: 151866333-151868333, Probe design region
(genomic position GRCh37:X: 151867273-151867394, nt 941 to nt 1062 of SEQ ID No. 212),
investigated CpG (genomic position GRCh37:X: 151867333, nt 1001 of SEQ ID No. 212)
Seq ID no. 213, RAB11FIP5 - GRCh37:2: 73338228-73340228, Probe design region
(genomic position GRCh37:2: 73339168-73339289, nt 941 to nt 1062 of SEQ ID No. 213),
investigated CpG (genomic position GRCh37:2: 73339228, nt 1001 of SEQ ID No. 213)
Seq ID no. 214, ARHGDIB - GRCh37:12: 15113394-15115394, Probe design region
(genomic position GRCh37:12: 15114333-15114454, nt 940 to nt 1061 of SEQ ID No. 214),
investigated CpG (genomic position GRCh37:12: 15114394, nt 1000 of SEQ ID No. 214)
Seq ID no. 215, EMILIN1 - GRCh37:2: 27300944-27302944, Probe design region (genomic
position GRCh37:2: 27301883-27302004, nt 940 to nt 1061 of SEQ ID No. 215), investigated
CpG (genomic position GRCh37:2: 27301944, nt 1000 of SEQ ID No. 215)
Seq ID no. 216, LPXN - GRCh37:11: 58341997-58343997, Probe design region (genomic
position GRCh37:11: 58342937-58343058, nt 941 to nt 1062 of SEQ ID No. 216),
investigated CpG (genomic position GRCh37:11: 58342997, nt 1001 of SEQ ID No. 216)
Seq ID no. 217, ALDH1A3 - GRCh37:15: 101419636-101421636, Probe design region
(genomic position GRCh37:15: 101420576-101420697, nt 941 to nt 1062 of SEQ ID No.
217), investigated CpG (genomic position GRCh37:15: 101420636, nt 1001 of SEQ ID No.
217)
Seq ID no. 218, SUHW1 - GRCh37:22: 22873678-22875678, Probe design region (genomic
position GRCh37:22: 22874617-22874738, nt 940 to nt 1061 of SEQ ID No. 218),
investigated CpG (genomic position GRCh37:22: 22874678, nt 1000 of SEQ ID No. 218)
Seq ID no. 219, GSTT1 - GRCh37:22: 24383160-24385160, Probe design region (genomic
position GRCh37:22: 24384099-24384220, nt 940 to nt 1061 of SEQ ID No. 219),
investigated CpG (genomic position GRCh37:22: 24384160, nt 1000 of SEQ ID No. 219)
Seq ID no. 220, Human LINE-1 transposon (L1Hs) DNA, GenBank accession n.: X58075,
assay design region (nt 111 to nt 358 of SEQ ID No. 220), investigated CpGs (nt 252, 256
and 270 of SEQ ID No. 220).

The following examples are intended to illustrate, but not to limit, the scope of the invention. The examples are illustrated by reference to the non limiting following figures.

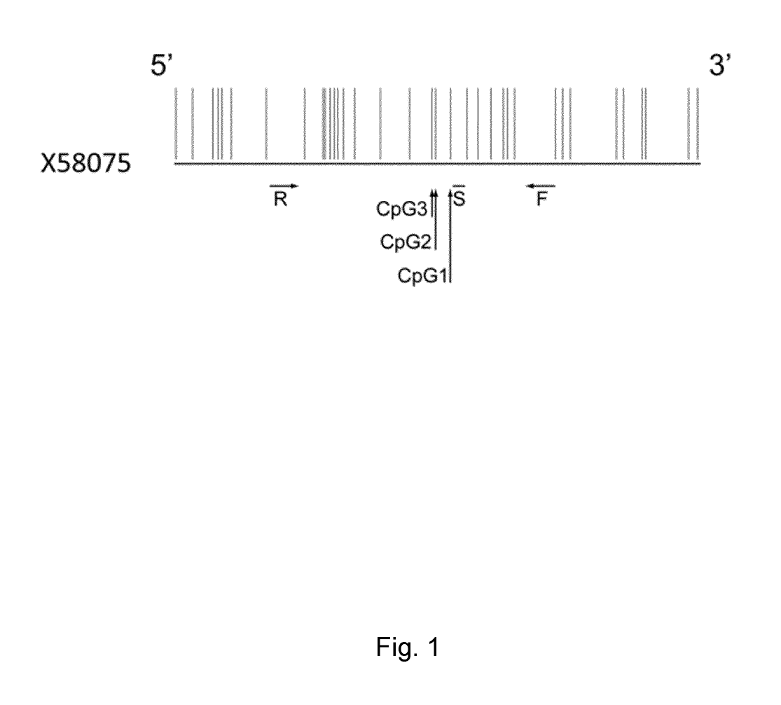

FIG. 1. LINE-1 bisulfite pyrosequencing assay map.

The region of the LINE-1 sequence (GenBank accession n. X58075) utilized for the design of the assay is reported. Vertical bars indicate individual CpG sites. Horizontal lines indicate forward (F), reverse (R) and sequencing (S) primers. Vertical arrows indicate the CpG sites (CpG1, CpG2, CpG3) analyzed by pyrosequencing (adapted from Aparicio et al, Epigenetics, 4:176-84, 2009).

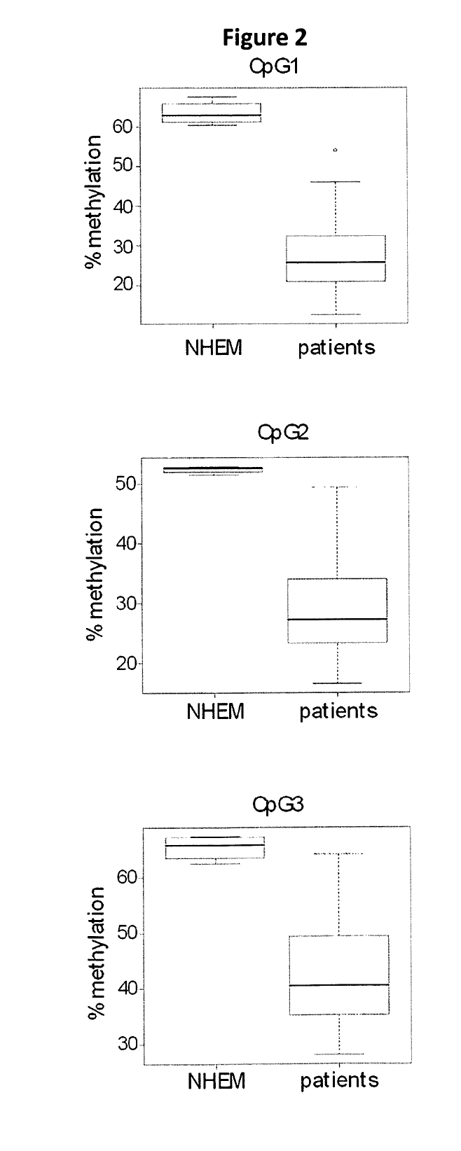

FIG. 2. LINE-(methylation in stage IIIC CM patients

LINE-1 methylation at 3 CpG sites (CpG1, CpG2, CpG3) was evaluated by bisulfite pyrosequencing analysis in 4 independent primary cultures of Normal Human Epidermal Melanocites (NREM) and inshort-term cultures of CM cells generated from neoplastic lesions of 42 stage IIIC melanoma patients. All cells were analyzed at 6^{th} in vitro passage. Separate box plots have been generated for each of the CpG sites under analysis. Black horizontal bars represent the median values of methylation for each group.

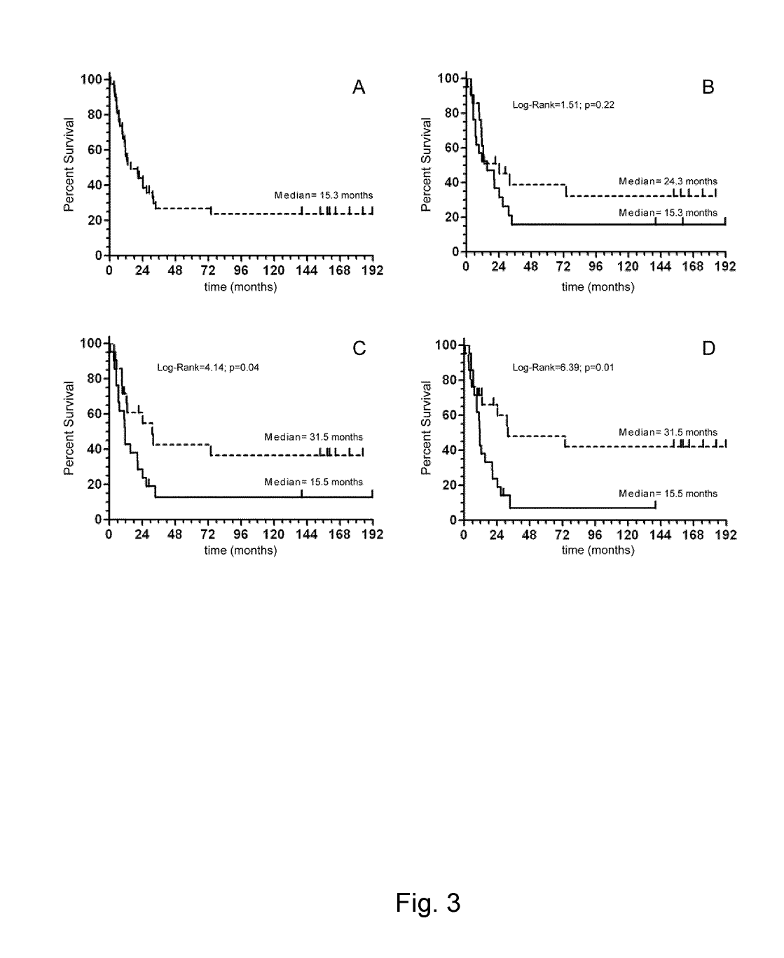

FIG. 3. Kaplan-Meier analysis of CM patients survival according to LINE-1 methylation

LINE-1 methylation at 3 CpG sites (CpG1, CpG2, CpG3) was evaluated by bisulfite pyrosequencing analysis in short-term cultures of CM cells generated from neoplastic lesions of 42 stage IIIC melanoma patients. Cells were analyzed at 6^{th} in vitro passage. Kaplan-Meier function for OS was calculated for CM patients either unstratified (A) or stratified according to median methylation of CpG1 (B), CpG2 (C) or CpG3 (D) site of LINE-1 elements. Dashed and solid lines refer to patients with LINE-1 methylation below or above the median, respectively. Vertical bars represent censored patients. Cumulative survival by LINE-1 methylation level was evaluated using the Log-Rank test, reported P values were two sided.

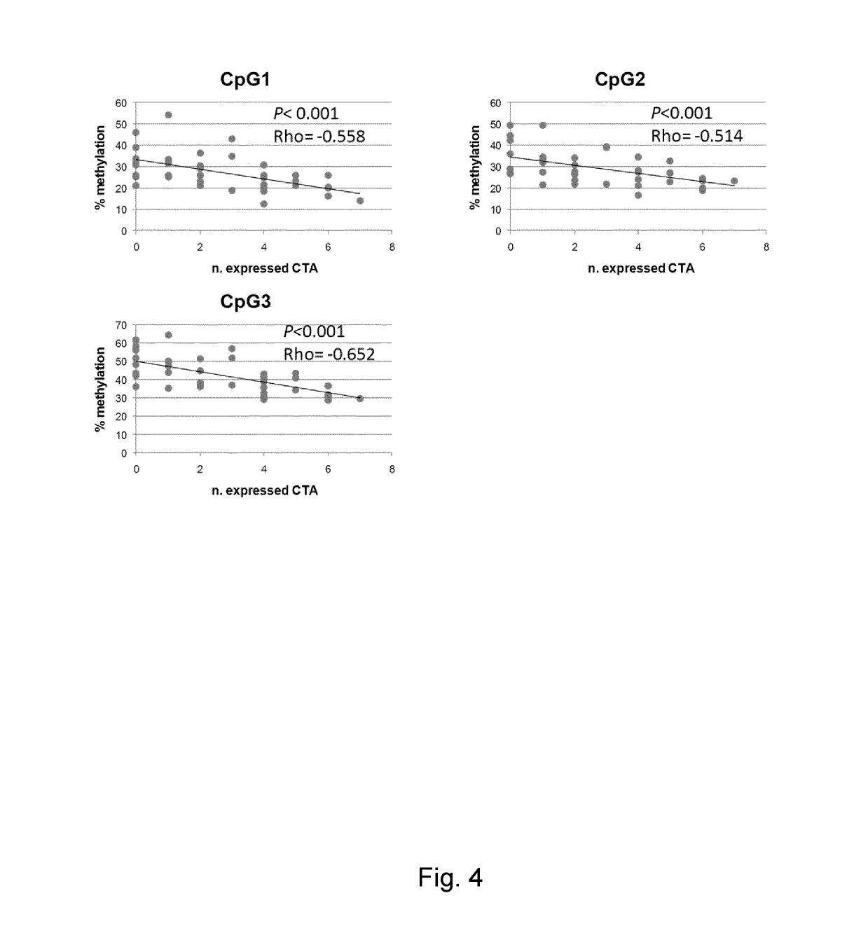

FIG. 4. LINE-1 methylation correlates with the number of CTA concomitantly expressed by CM cells.

LINE-1 methylation at 3 CpG sites (CpG1, CpG2, CpG3) was evaluated by bisulfite pyrosequencing analysis in primary cultures of CM cells at the 6^{th} in vitro passage. Total RNA was extracted from the same cell cultures and subjected to RT-PCR analyses for the expression of the CTA MAGE-AL-A2, -A3, -A4, -A10, SSX-2 and NY-ESO-1. For each cell culture, the number of different CTA expressed was evaluated. The correlation between LINE-1 methylation at each CpG site and the number of expressed CTA was evaluated through Spearman's rank correlation test. P values<0.05 were considered to be statistically significant.

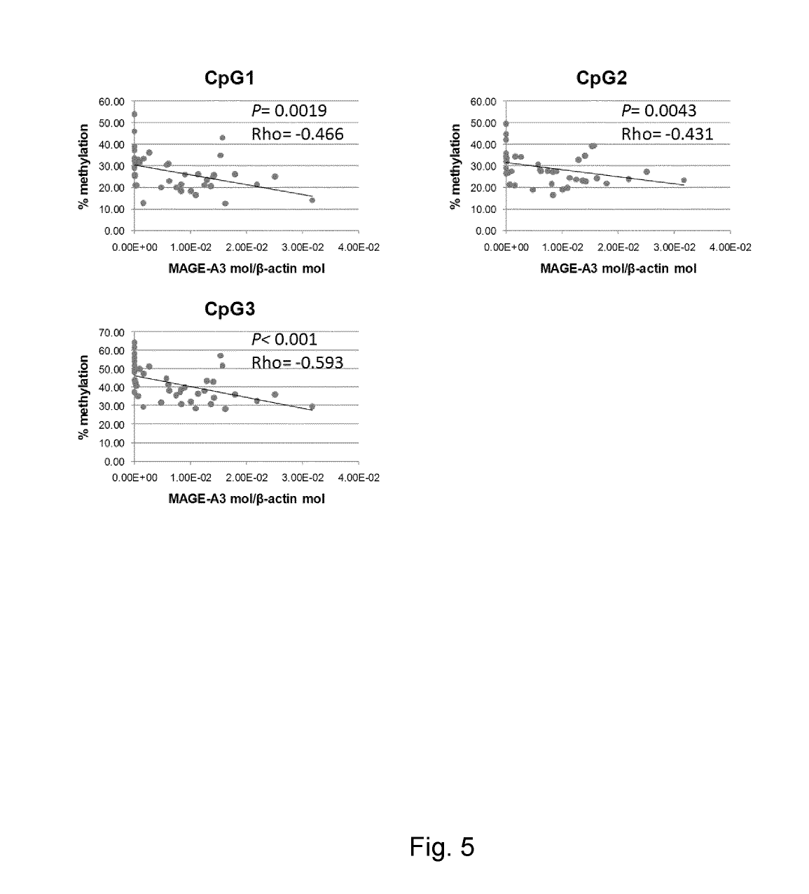

FIG. 5. LINE-1 methylation correlates with the quantitative expression of MAGE-A3 by CM cells.

LINE-1 methylation at 3 CpG sites (CpG1, CpG2, CpG3) was evaluated by bisulfite pyrosequencing analysis in primary cultures of CM cells at the 6^{th} in vitro passage. Total RNA was extracted from the same cell cultures and subjected to quantitative RT-PCR analysis for the expression of the CTA MAGE-A3 and of the house-keeping gene (3-actin. MAGE-A3 expression in each sample was normalized to that of (3-actin, and reported as MAGE-A3 molecules/β-actin molecules. The correlation between LINE-1 methylation at each CpG site and the quantitative levels of expression of MAGE-A3 was evaluated through Spearman's rank correlation test. P values<0.05 were considered to be statistically significant.

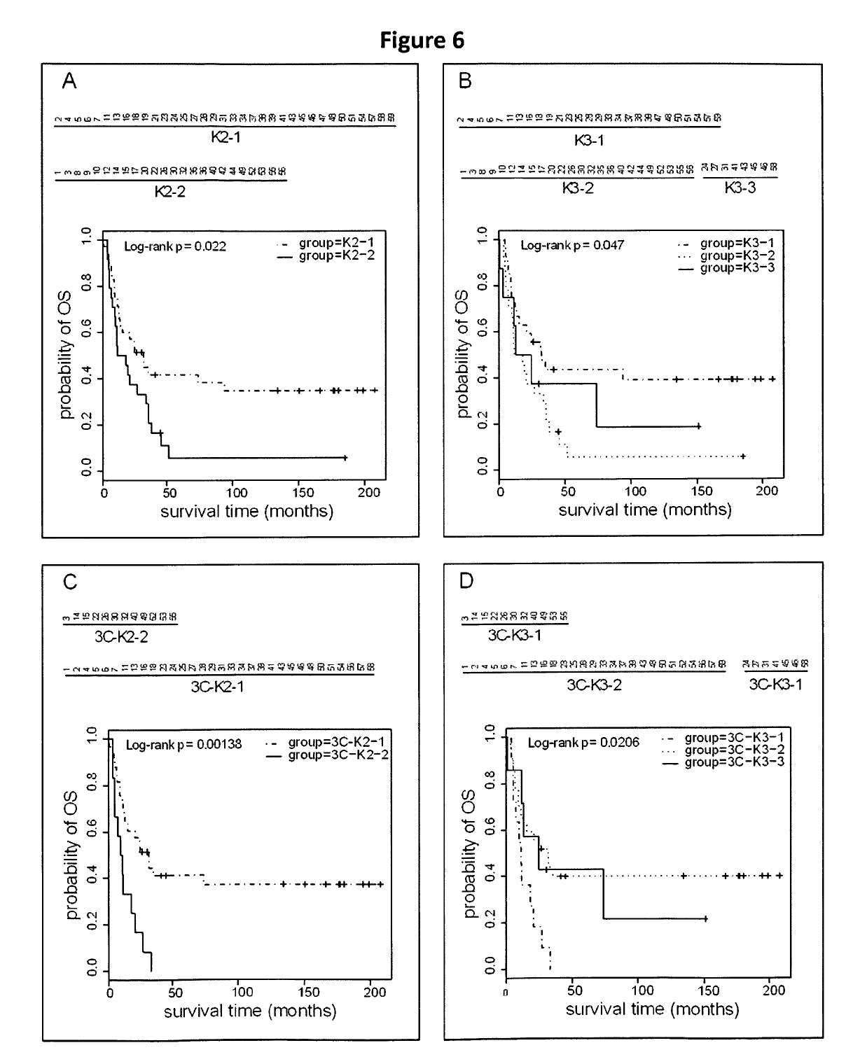

FIG. 6. Kaplan-Meier analysis of CM patients survival according to genome-wide methylation profiles

Genome-wide DNA methylation profile was evaluated by Illumina HumanMethylation27 Bead-Chip whole-genome assay in short-term cultures of CM cells generated from neoplastic lesions of stage III CM patients. Cells were analyzed at 6^{th} in vitro passage. Patients were divided in either 2 or 3 classes based on the whole genome methylation profile of their tumor cells through the k-means algorithm. Kaplan-Meier function for OS was calculated for stage III and stage IIIC CM patients stratified according to k-means-defined methylation classes. Vertical bars in the Kaplan-Meier curves represent censored patients. Cumulative survival by k-means-defined methylation group was evaluated using the Log-Rank test, reported P values were two sided. Numbers above the horizontal bars in the upper portion of panels A-D represent the patients' numeric identifiers, horizontal bars and text below identify the k-means-defined methylation classes. Panel A, B, stage III patients stratified according to two (K2-1, K2-2) or three (K3-1, K3-2, K3-3) k-means-defined methylation classes, respectively; Panel C, D stage IIIC patients stratified according to two (3C-K2-1, 3C-K2-2) or three (3C-K3-1, 3C-K3-2, 3C-K3-3) k-means-defined methylation classes, respectively.

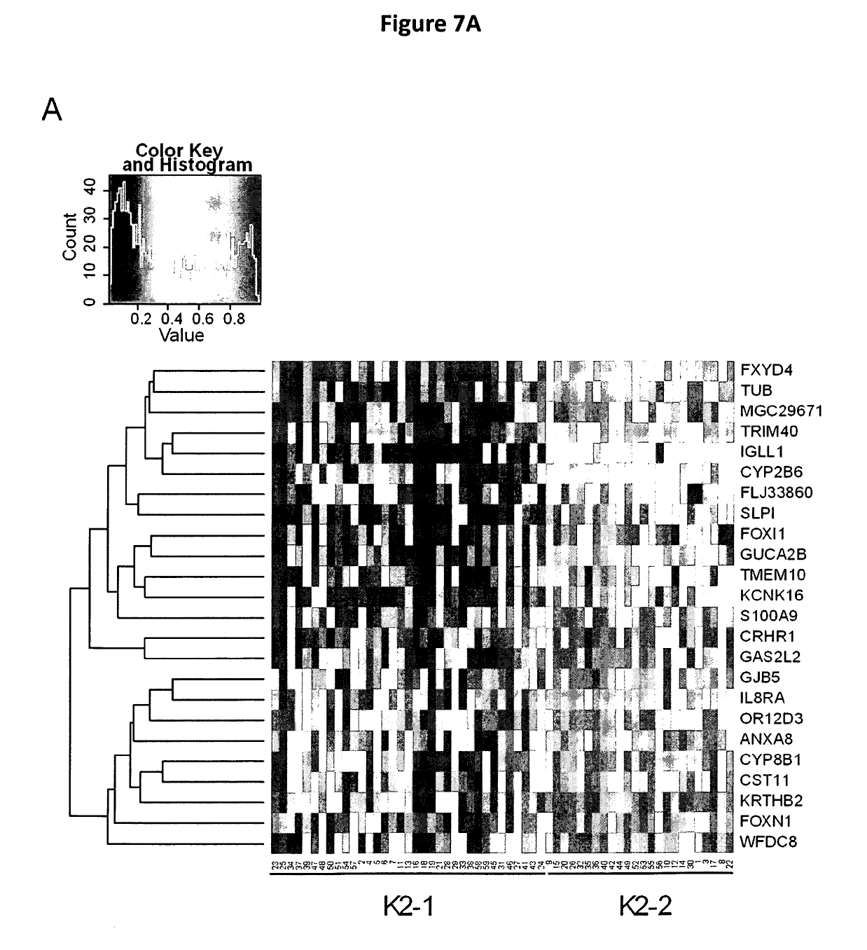

FIG. 7. Prognostic methylation signatures identified by the “nearest shrunken centroid” algorithm in stage III CM patients.

Pre-processed genome-wide methylation data from stage III CM patients under study was analyzed through nearest shrunken centroids algorithm to identify genes best characterizing the k-means defined prognostic methylation groups. The methylation values of the genes composing the methylation signatures in the patients' population have been reported as a heatmap. Genes defining the signature have been reported as Gene Symbol on the right of the heatmap, while patient identifier numbers and the k-means-defined classes have been reported on foot of the heatmap. Each color patch represents the methylation level of one gene in each patient, with a continuum of methylation levels from dark blue (completely unmethylated, β=0) to dark red (completely methylated, β=1). Panel A, 24 genes signature classifying patients into 2 k-means-defined classes (K2-1 and K2-2) with an overall error rate of 0.05; Panel B, 98 genes signature classifying patients into 3 k-means-defined classes (K3-1, K3-2, and K3-3) with an overall error rate of 0.034.

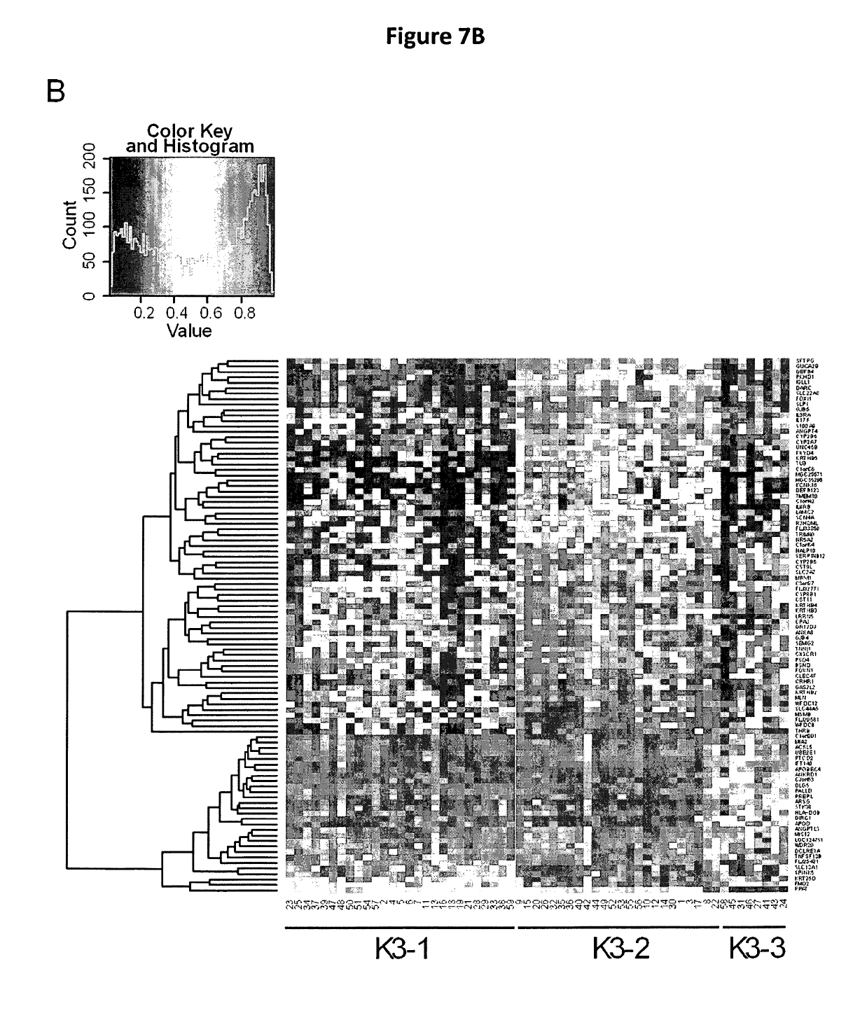

FIG. 8. Prognostic methylation signatures identified by the “nearest shrunken centroid” algorithm in stage IIIC CM patients.

Pre-processed genome-wide methylation data from stage IIIC CM patients under study was analyzed through nearest shrunken centroids algorithm to identify genes best characterizing the k-means defined prognostic methylation groups. The methylation values of the genes composing the methylation signatures in the patients' population have been reported as a heatmap. Genes defining the signature have been reported as Gene Symbol on the right of the heatmap, while patient identifier numbers and the k-means-defined classes have been reported on foot of the heatmap. Each color patch represents the methylation level of one gene in each patient, with a continuum of methylation levels from dark blue (completely unmethylated, β=0) to dark red (completely methylated, β=1). Panel A, 17 genes signature classifying patients into 2 k-means-defined classes (3C-K2-1 and 3C-K2-2) with an overall error rate of 0; Panel B, 47 genes signature classifying patients into 3 k-means-defined classes (3C-K3-1, 3C-K3-2, and 3C-K3-3) with an overall error rate of 0.022.

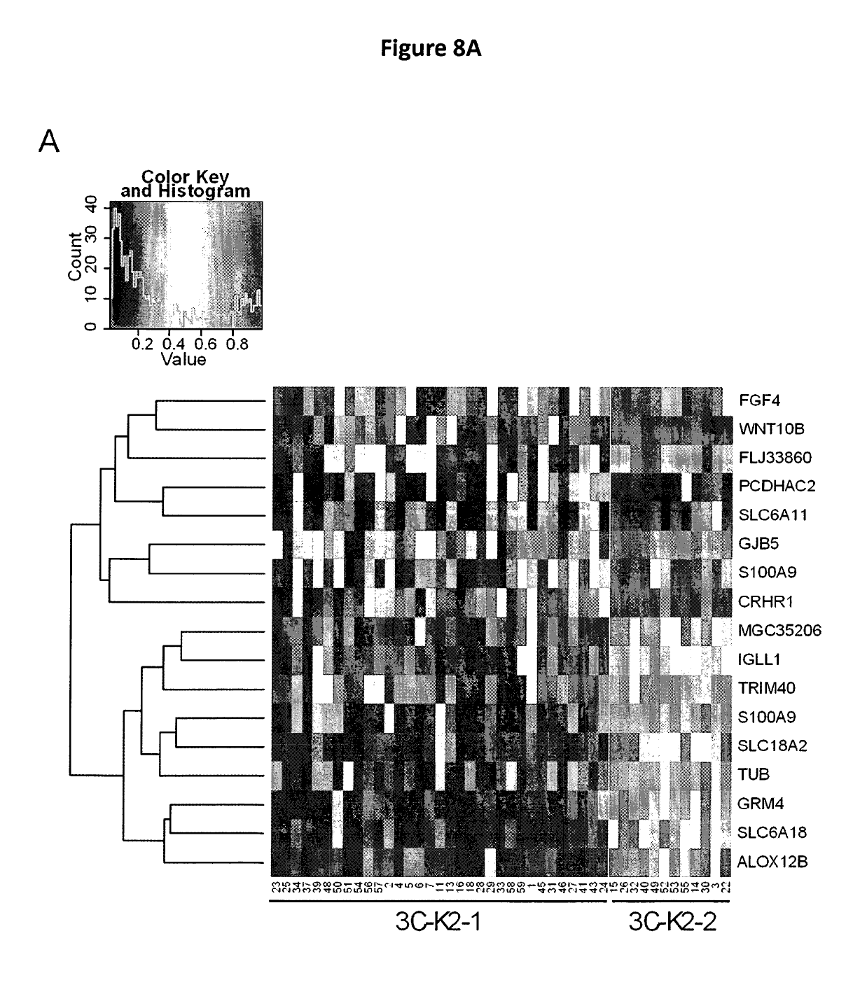

FIG. 9. Prognostic methylation signatures identified by the “nearest shrunken centroid” algorithm in stage IIIC CM patients using correlation between gene methylation and gene expression (p<0.05).

Pre-processed genome-wide methylation data from stage IIIC CM patients under study was analyzed through nearest shrunken centroids algorithm to identify genes best characterizing the k-means defined prognostic methylation groups. The analysis has been restricted to 820 methylation probes showing significant association between gene methylation and gene expression values in the same patient population, as evaluated by Spearman test. The methylation values of the genes composing the methylation signatures in the patients' population have been reported as a heatmap. Genes defining the signature have been reported as Gene Symbol on the right of the heatmap, while patient identifier numbers and the k-means-defined classes have been reported on foot of the heatmap. Each color patch represents the methylation level of one gene in each patient, with a continuum of methylation levels from dark blue (completely unmethylated, (3=0) to dark red (completely methylated, (3=1). Duplicate Gene Symbol entries refer to methylation data read for the same gene from different probes. Panel A, 51 genes signature classifying patients into 2 k-means-defined classes (3C-K2-1 and 3C-K2-2) with an overall error rate of 0.022; Panel B, 73 genes signature classifying patients into 3 k-means-defined classes (3C-K3-1, 3C-K3-2, and 3C-K3-3) with an overall error rate of 0.

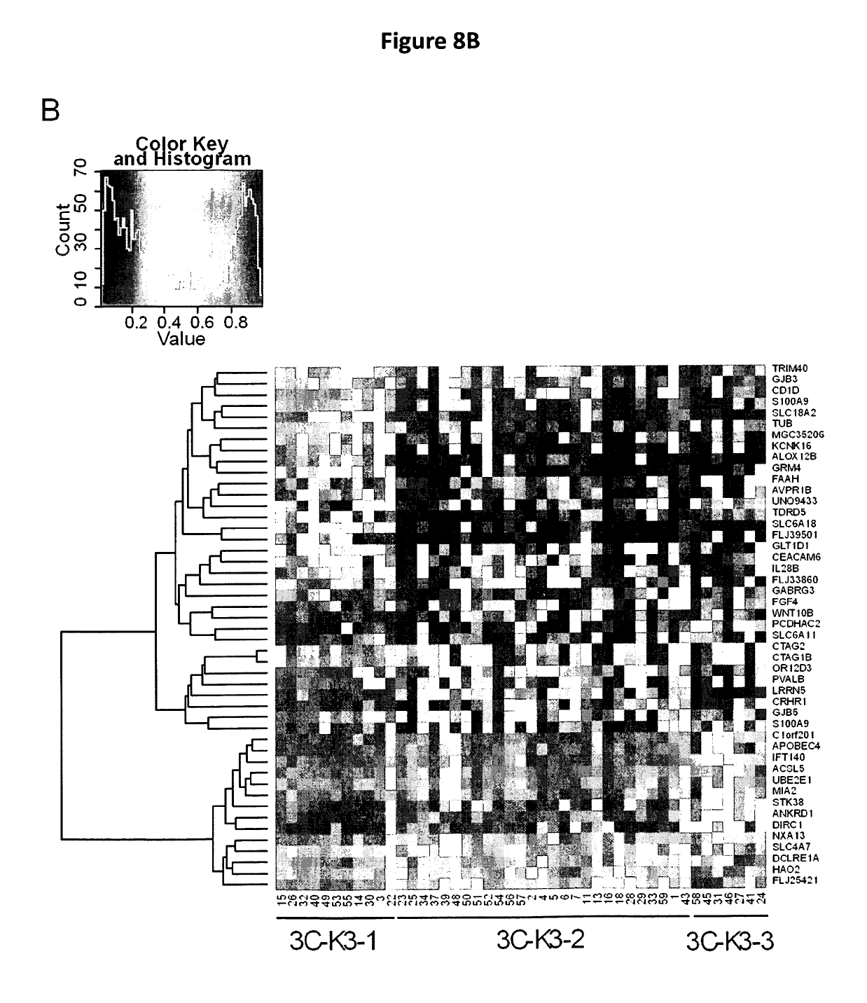

FIG. 10. Prognostic methylation signatures identified by the “nearest shrunken centroid” algorithm in stage IIIC CM patients using correlation between gene methylation and gene expression (p≦0.05, Rho≦−0.4).

Pre-processed genome-wide methylation data from stage IIIC CM patients under study was analyzed through nearest shrunken centroids algorithm to identify genes best characterizing the k-means defined prognostic methylation groups. The analysis has been restricted to 274 methylation probes showing significant (p≦0.05) association between gene methylation and gene expression values in the same patient population, and with a coefficient of correlation Rho≦−0.4, as evaluated by Spearman test. The methylation values of the genes composing the methylation signatures in the patients' population have been reported as a heatmap. Genes defining the signature have been reported as Gene Symbol on the right of the heatmap, while patient identifier numbers and the k-means-defined classes have been reported on foot of the heatmap. Each color patch represents the methylation level of one gene in each patient, with a continuum of methylation levels from dark blue (completely unmethylated, β=0) to dark red (completely methylated, β=1). Duplicate Gene Symbol entries refer to methylation data read for the same gene from different probes.

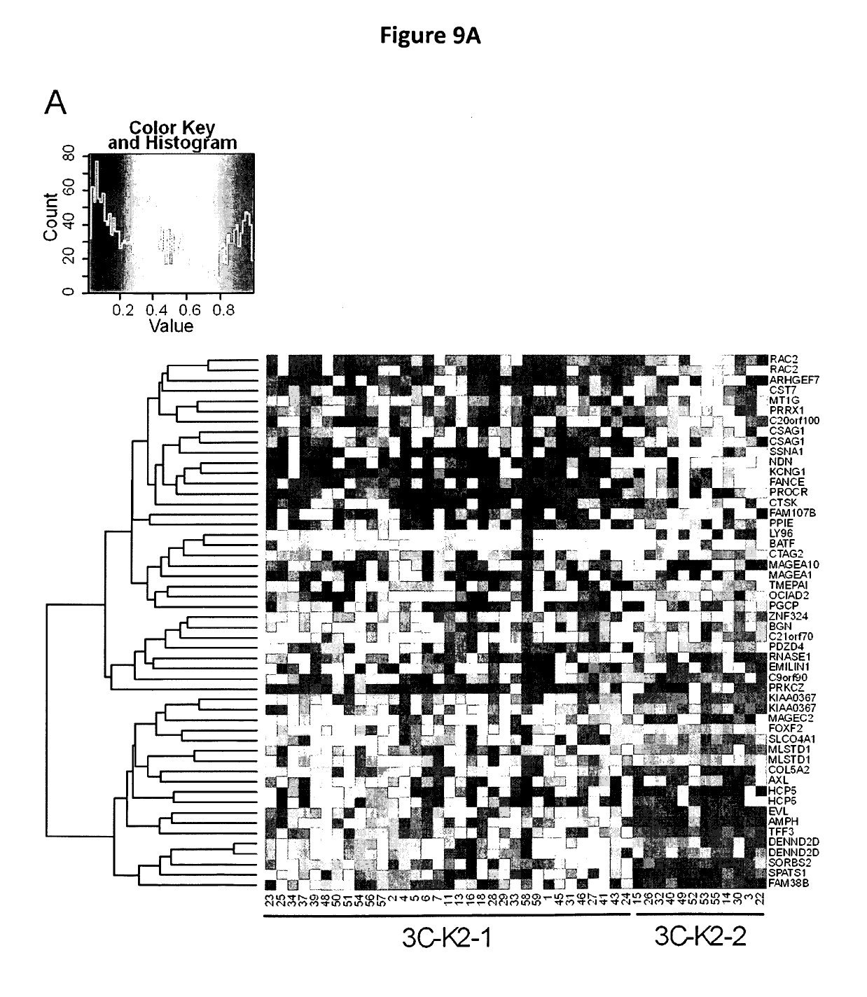

FIG. 11. Kaplan-Meier analysis of stage IIIC CM patients survival according to FGF4, CTAG2, OCIAD2 or PAGE5 methylation status.

Genome-wide DNA methylation profile was evaluated by Illumina HumanMethylation27 Bead-Chip whole-genome assay in short-term cultures of CM cells generated from neoplastic lesions of stage IIIC CM patients. Cells were analyzed at 6^{th} in vitro passage. The methylation level of the genesFGF4 (panel A), CTAG2 (panel B), OCIAD2 (panel C) and PAGES (panel D), selected from the whole-genome defined prognostic methylation signatures E and G, have been utilized to subdivide patients according to the methylation level of each gene being<33%, between 33% and 66%, or >66%. Kaplan-Meier function for OS was calculated for stage IIIC patients stratified according to the methylation level of each gene. Vertical bars in the Kaplan-Meier curves represent censored patients. Cumulative survival by methylation group was evaluated using the Log-Rank test, reported P values were two sided.

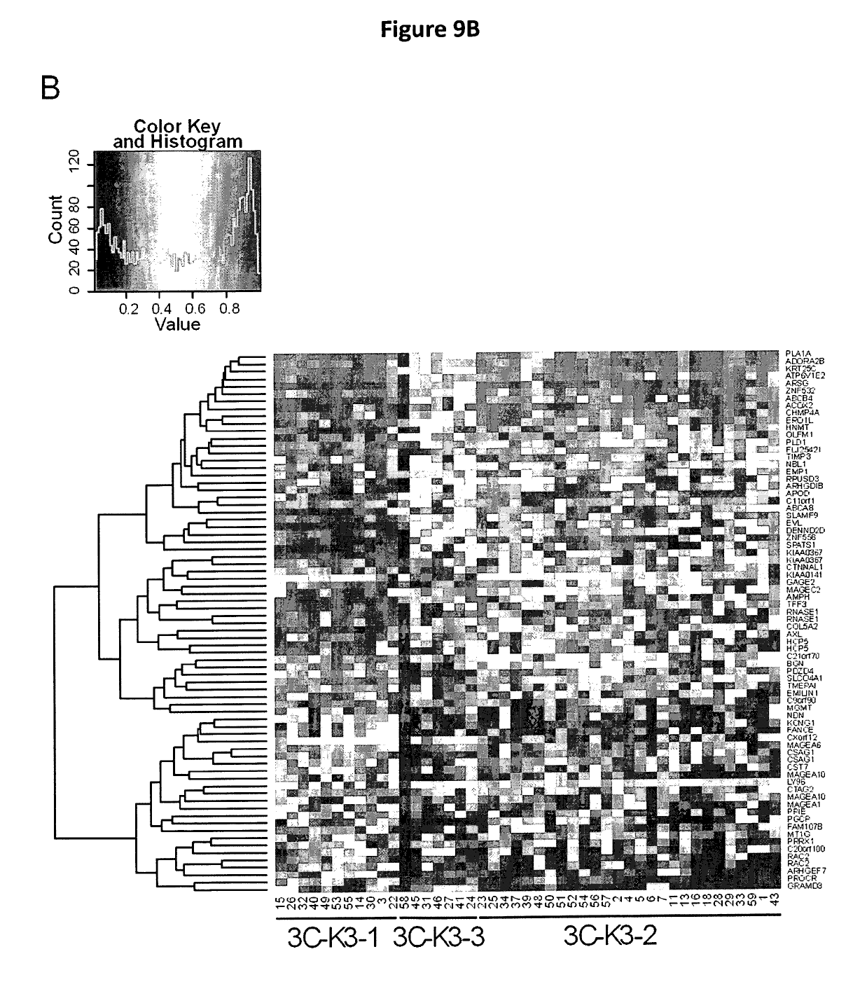

FIG. 12. Kaplan-Meier analysis of CM patients survival according to genome-wide methylation profiles and LINE-1 methylation status

Genome-wide DNA methylation profile was evaluated by Illumina HumanMethylation27 Bead-Chip whole-genome assay in short-term cultures of CM cells generated from neoplastic lesions of stage III CM patients. Cells were analyzed at 6^{th} in vitro passage. Patients were scored as K2-1 or k2-2 (stage III patients) or as S3-K2-1 or S3-K2-2 (substage IIIC patients) based on the whole genome methylation profile of their tumor cells through the k-means algorithm. Concomitantly, short-term cultures of CM cells were evaluated for the extent of LINE-1 methylation by pyrosequencing, and scored as hypomethylated or hypermethylated depending on their value being< or ≧ median value, respectively. Kaplan-Meier function for OS was calculated for stage III (panel A) and stage IIIC (panel B) CM patients stratified according to both genome-wide methylation profile-defined classes and LINE-1 methylation. Vertical bars in the Kaplan-Meier curves represent censored patients. Cumulative survival by methylation group was evaluated using the Log-Rank test, reported P values were two sided.

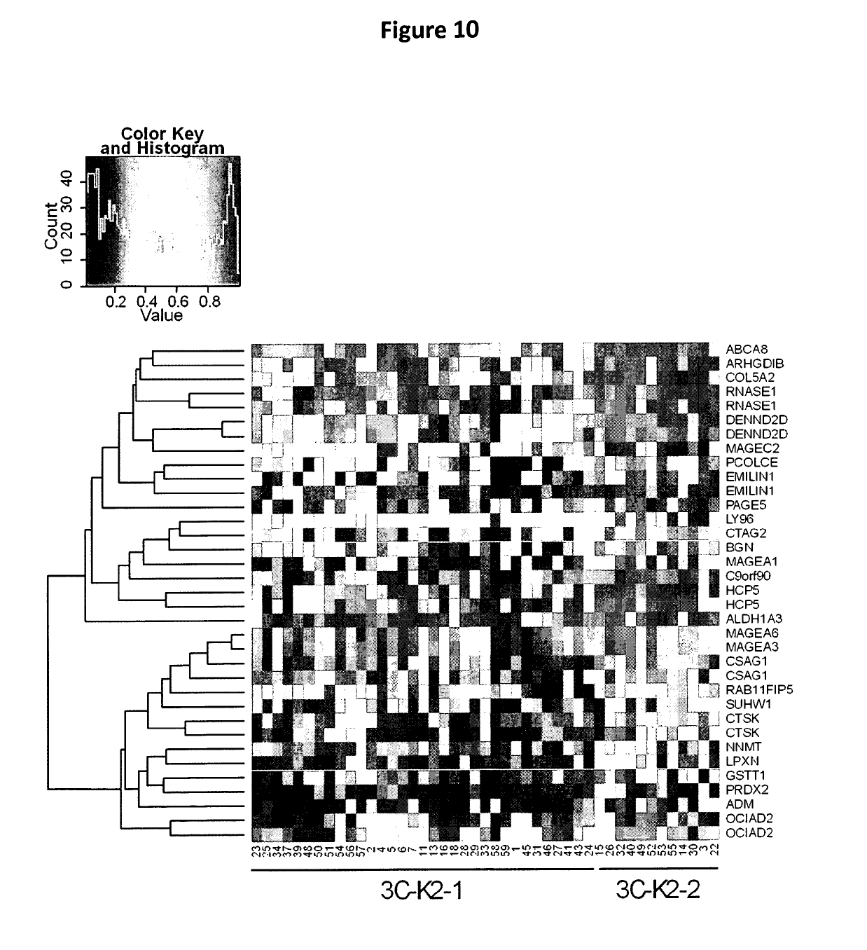

FIG. 13. Kaplan-Meier analysis of CM patients survival according to genome-wide methylation profile defined “methylation score”. Genome-wide DNA methylation profile was evaluated by Illumina HumanMethylation27 Bead-Chip whole-genome assay in short-term cultures of CM cells generated from neoplastic lesions of stage III CM patients. Cells were analyzed at 6^{th} in vitro passage. The genome-wide methylation profile of each patient was summarized by the “methylation score”. Patients were divided into 2 classes based on the “methylation score” of their tumor cells being<(dotted line) or ≧(solid line) the median value of the patients' population. Kaplan-Meier function for OS was calculated for stage 111C CM patients stratified according to the “methylation score”. Vertical bars in the Kaplan-Meier curves represent censored patients. Cumulative survival by “methylation score”-defined groups was evaluated using the Log-Rank test, reported P values were two sided.

### DETAILED DESCRIPTION OF THE INVENTION

### Example 1

### Methylation Levels of the “Long Interspersed Nucleotide Element-1” Repetitive Sequences Predict Survival of Melanoma Patients

The prognosis of cutaneous melanoma (CM) differs for patients with identical clinico-pathological stage, and no molecular markers discriminating the prognosis of stage III individuals have been established. Genome-wide alterations in DNA methylation are a common event in cancer. This example defines the prognostic value of genomic DNA methylation levels in stage III CM patients.

Overall level of genomic DNA methylation was measured using bisulfite pyrosequencing at three CpG sites (CpG1, CpG2, CpG3) of the Long Interspersed Nucleotide Element-1 (LINE-1) sequences in short-term CM cultures from 42 stage IIIC patients. The impact of LINE-1 methylation on overall survival (OS) was assessed using Cox regression and Kaplan-Meier analysis.

Here the authors provide the first evidence that the methylation level of LINE-1 repetitive elements has a significant prognostic value in stage IIIC CM patients. Specifically, patients whose tumors exhibit more hypomethylated LINE-1 sequences had a significantly longer overall survival. The reported data have significant prospective practical clinical implications. Indeed, it can be expected that the evaluation of LINE-1 methylation status will: i) provide CM patients with improved clinico-pathological sub-staging information; ii) allow physicians to personalize follow-up procedures for CM patients; iii) guide the future selection and/or stratification of patients for trials of adjuvant treatment; iv) select patients who are most likely to benefit from therapy, including DNA hypomethylating drugs. Overall, the evaluation of LINE-1 methylation in the routine clinico-pathological ascertainment of CM patients is anticipated to greatly help in personalizing their comprehensive clinical management.

### Materials and Methods

### Patients and Cell Cultures

Short-term cell cultures were established from metastatic lesions removed surgically from consecutive CM patients referred to the National Cancer Institute of Aviano (Italy) for stage III surgery from 1991 to 2007, as previously described (Altomonte et al., 1993). Autologous tumor cell cultures were successfully established from 30% of patients. The micrometastatic nature of lymph-node tumor tissues from AJCC stage IIIA patients precluded their use for cell culture generation, while short-term CM cultures were available only from 12 stage TIM patients, and were excluded from the statistical analyses. Thus, the planned studies were conducted on a total of 42 available short-term cultures, identified as having been generated from CM patients classified as AJCC stage IIIC. Short-term CM cell cultures were grown in RPMI 1640 Medium (Biochrome KG, Berlin, Germany) supplemented with 20% heat-inactivated fetal calf serum (Biochrome KG) and 2 mM L-glutamine (Biochrome KG). Four independent cultures of NREM were purchased from Invitrogen (Milan, Italy), Gentaur (Brussels, Belgium), Provitro (Berlin, Germany), and ScienCell (Carlsbad, Calif., USA), and were maintained in M254 Medium supplemented with Human Melanocyte Growth Supplement (Invitrogen). To minimize alterations potentially arising with extended in vitro culturing, all cell cultures were utilized for molecular assays at the 6^{th} ex vivo passage.

### LINE-1 Bisulfite Pyrosequencing Analysis

Genomic DNA was extracted from short-term cultures of CM cells by proteinase K treatment followed by standard phenol/chloroform extraction and ethanol precipitation (Ausubel et al., 1998). Bisulfite conversion was carried out on 500 ng genomic DNA using EZ DNA Methylation-Gold™ Kit (Zymo Research, Orange, Calif., USA), according to the manufacturer's protocol. Methylation analysis of the LINE-1 elements was performed as previously described (Yang et al., 2004), with minor modifications. LINE-1 elements were amplified using 50 pmol each of forward primer 5′-TTTTTTGAGTTAGGTGTGGG-3′ (SEQ ID No. 221) and reverse biotinylated primer 5′-TCTCACTAAAAAATACCAAACAA-3′ (SEQ ID No. 222) in a 50 μL reaction volume containing 2.5 ng of bisulfite-treated DNA, 1×PCR buffer, 1.5 mM MgCl₂ and 1.25 U of Platinum Taq DNA polymerase (Invitrogen). PCR thermal amplification profile consisted of an initial denaturation step of 5 min at 95° C., followed by 50 cycles of 30 s at 95° C., 30 s at 58° C., and 1 min at 72° C. The PCR product was purified using Streptavidin Sepharose High Performance beads (Amersham Biosciences, Uppsala, Sweden) and denatured using 0.2 mol/L of NaOH solution. Next, 0.3 μmol/L of the sequencing primer (5′-GGGTGGGAGTGAT-3′) (SEQ ID No. 223) was annealed to the purified single-stranded PCR product and the Pyrosequencing reaction was performed using the PSQ HS 96 Pyrosequencing System (Pyrosequencing, Inc., Westborough, Mass.). The level of methylation for each of the 3 analyzed CpG sites (CpG1, CpG2, CpG3) was expressed as the percentage of methylated cytosines over the sum of methylated and unmethylated cytosines (FIG. 1).

### Statistical Analysis

The primary objective was to determine differences in survival among various LINE-1 DNA methylation level groups. LINE-1 DNA methylation levels were dichotomized using median value into the following categories: CpG1 (<25.68, ≧25.68), CpG2 (<27.26, ≧27.26), and CpG3 (<40.46, ≧40.46). The characteristics including age, gender, primary tumor localization, Breslow thickness, Clark level, and ulceration of the primary tumor, number of lymph nodes involved, and pre-operative serum LDH values were examined. Survival time was calculated in months from the date of stage IIIC diagnosis until the date of death. According with the specific goals of the analysis, the authors did not classify the deaths considering their cause. Patients were censored at the last follow-up date or the last date the patient was last known to be alive. Median survival duration was determined by the Kaplan-Meier method (Kaplan and Meier, 1958). Cumulative survival by DNA methylation level was evaluated using the log-rank test. P values were two sided and values<0.05 were considered to be statistically significant. Cox proportional hazard method (Cox, 1972) was used to examine the effect of DNA methylation level on survival and results were presented as Hazard Ratios (HR) with corresponding 95% Confidence Intervals (CI). LINE-1 methylation was also entered in the model as a continuous variable with the unit set at 10% of methylation. Patient characteristics and clinical and pathologic features were examined by univariate Cox model. The statistical analyses were carried out using the SAS Software version 9.13 (SAS Institute Inc., Cary, N.C., USA).

### Results

### Patients

The study was conducted on CM patients who underwent radical lymph node dissection for stage III disease at the Centro di Riferimento Oncologico National Cancer Institute between 1991 and 2007. Patients diagnosed with a stage IIIC disease, and for whom a short-term cell culture had been successfully generated from the surgically removed autologous neoplastic tissue, were included in the study. Table 5 summarizes the 42 patients under study and their clinico-pathologic characteristics at presentation.

TABLE 5
Characteristics of the 42 AJCC stage IIIC melanoma patients

| Variable | n. patients | % |
|---|---|---|
| Age, years | | |
| Median | 54 | |
| Range | 29-83 | |
| Gender | | |
| Male | 27 | 64 |
| Female | 15 | 36 |
| Localization of primary tumor | | |
| extremities | 14 | 33 |
| trunk | 23 | 55 |
| head & neck | 3 | 7 |
| NA* | 2 | 5 |
| Breslow thickness of primary tumor | | |
| ≦2.0 mm | 13 | 31 |
| >2.0 mm | 22 | 52 |
| NA | 7 | 17 |
| Clark level of primary tumor | | |
| 1-3 | 12 | 29 |
| 4-5 | 24 | 57 |
| NA | 6 | 14 |
| Ulceration of primary tumor | | |
| No | 10 | 24 |
| Yes | 30 | 71 |
| NA | 2 | 5 |
| N. lymph nodes involved | | |
| 1 | 9 | 21 |
| >1 | 33 | 79 |
| LDH | | |
| Low_{†} | 28 | 67 |
| High | 11 | 26 |
| NA | 3 | 7 |

*NA, not available
_{†}low LDH is established as LDH values ≦ 0.8 times the upper limit of normal, high LDH is established as LDH values > 0.8 times the upper limit of normal

### Extent of LINE-1 Methylation in CM Patients

To define if CM undergoes changes in the overall content of 5-methylcytosine, bisulfite pyrosequencing analyses were utilized to compare the extent of methylation of LINE-1 repetitive elements in the 42 short-term CM cell cultures under study to that of primary cultures of NREM. LINE-1 sequences were heavily methylated at all investigated CpG sites in NREM (CpG1: median 62.82%, range 60.43%-67.53%; CpG2: median 52.57%, range 51.37%-52.87%; CpG3: median 65.77%, range 62.40%-67.33%; FIG. 2).

On the other hand, much lower and largely heterogeneous levels of methylation of the LINE-1 elements were observed in CM cells from stage IIIC patients (CpG1: median 25.68%, range 12.45%-54.05%; CpG2: median 27.26%, range 16.50%-49.43%; CpG3: median 40.46%, range 28.10%-64.15%; FIG. 2), demonstrating that highly variable alterations in the overall level of genomic DNA occur in CM.

### Prognostic Value of LINE-1 Methylation in CM Patients

The highly heterogeneous levels of LINE-1 methylation observed in CM cells from stage IIIC patients led us to investigate whether they correlated with a different clinical outcome of patients under study.

Kaplan-Meier analysis indicated that median OS for stage IIIC CM patients under analysis was 15.3 months (95% CI, 11.0-31.5; FIG. 3A). To evaluate the association between LINE-1 methylation status and OS, patients were divided according to the median value of methylation of each analyzed CpG site (CpG1=25.68%; CpG2=27.26%; CpG3=40.46%). Patients were defined as having hypomethylated or hypermethylated LINE-1 sequences, depending on the methylation level being below or above the median value for each group, respectively. Kaplan-Meier analysis showed a trend towards an increased OS rate for patients with hypomethylated CpG1, however, the difference did not reach statistical significance (P=0.22, log-rank=1.51; FIG. 3B). On the other hand, a significant survival advantage was observed in patients with CpG2<27.26% as compared to patients with CpG2>27.26% (P=0.04, log-rank=4.14) (FIG. 3C). Similarly, the survival rate of patients with CpG3<40.46% was significantly higher than that of patients with CpG3>40.46% (P=0.01, log-rank=6.39) (FIG. 3D). In line with these data, median OS of patients with hypomethylated CpG1, CpG2 and CpG3 sites was 24.3, 31.5, and 31.9 months, respectively, as compared to 15.3, 11.5, and 11.5 months of patients with hypermethylated LINE-1 CpGs (FIG. 3, Table 6).

TABLE 6
OS of stage IIIC CM patients according to LINE-1 methylation

| LINE1 | Extent | | | |
|---|---|---|---|---|
| CpG site | methylation* | Median OS (95% CI)^{†} | | 5 year OS (%) |
| CpG1 | <25.68 | 24.3 | (11.1-inf) | 39 |
| | ≧25.68 | 15.3 | (6.8-26.9) | 16 |
| CpG2 | <27.26 | 31.5 | (12.5-inf) | 43 |
| | ≧27.26 | 11.5 | (6.8-20.9) | 13 |
| CpG3 | <40.46 | 31.9 | (13.1-inf) | 48 |
| | ≧40.46 | 11.5 | (9.2-20.6) | 7 |

*Patients were divided according to the % of methylation of the specified CpG site being < or ≧ the median % methylation measured in the examined patients' population;
^{†}Survival functions were calculated by the Kaplan-Meier method. Data are reported as Median OS in months, together with the corresponding 95% Confidence Intervals (CI).

Accordingly, the 5 year OS was 39%, 43%, and 48% for patients with hypomethylated CpG1, CpG2, and CpG3 sites, respectively, as compared to 16%, 13%, and 7% of patients with hypermethylated LINE-1 CpGs (Table 5).

Cox univariate analysis was carried out to identify patient characteristics and clinico-pathologic factors that predicted survival. Among all factors examined, including age, gender, localization of primary tumor, Breslow thickness, Clark level and ulceration of primary tumor, number of lymph nodes involved, and level of pre-operative LDH, only CpG2 methylation (HR=2.12 for CpG2≧27.26% vs. CpG2<27.26; 95% CI: 1.01-4.44; P=0.04) and CpG3 methylation (HR=2.63 for CpG2>40.46% vs. CpG2<40.46; 95% CI: 1.21-5.69; P=0.01) were associated with statistically significant differences in OS (Table 7). When LINE-1 methylation was analyzed as a continuous variable, a statistically significant inverse association emerged between OS and an increase of 10% of methylation of CpG1 (HR=1.51; 95% CI:1.06-2.16; P=0.02), CpG2 (HR=1.60; 95% CI:1.02-2.52; P=0.04) and CpG3 (HR=1.49; 95% CI:1.06-2.09; P=0.02) (Table 7).

TABLE 7
Cox analysis of the influence of LINE-1 methylation
on OS of stage IIIC CM patients

| LINE1 | Extent | | | | |
|---|---|---|---|---|---|
| CpG | methyl- | | 95% CI; | | 95% CI; |
| site | ation* | HR^{†} | P value | HR_{cont.}^{‡} | P value |
| CpG1 | <25.68 | 1^{§} | | 1.51 | 1.06-2.16; 0.02 |
| | ≧25.68 | 1.57 | 0.76-3.24; 0.22 | | |
| CpG2 | <27.26 | 1 | | 1.60 | 1.02-2.52; 0.04 |
| | ≧27.26 | 2.12 | 1.01-4.44; 0.04 | | |
| CpG3 | <40.46 | 1 | | 1.49 | 1.06-2.09; 0.02 |
| | ≧40.46 | 2.63 | 1.21-5.69;. 0.01 | | |

*Patients were divided according to the % of methylation of the specified CpG site being < or ≧ the median % methylation measured in the examined patients' population;
^{†}Cox proportional hazard method was used to examine the effect of LINE-1 methylation on OS. Results were presented as Hazard Ratios (HR) with corresponding 95% Confidence Intervals (CI);
^{‡}LINE-1 methylation was also evaluated as continuous variable. The HR value is that of the LINE-1 methylation relative to an increase of 10%;
^{§}set as reference.

In the present invention, the authors demonstrate that the global level of LINE-1 methylation of short-term tumor cell cultures grown from patients with nodal disease is a significant predictor of OS in stage IIIC CM patients. This finding is of remarkable clinical relevance, since, to the best of our knowledge, it provides the first evidence of a molecular marker capable of differentiating the prognosis of CM patients in this high-risk substage. These results are of particular emphasis given the conduct of this study in subjects within a single clinically well-defined clinico-pathological staging sub-group, which has become the focus of several ongoing clinical trials in the US and Europe (i.e., ECOG intergroup trial E4697, EORTC trial 18071, GSK trial 111482 “DERMA”).

Genomic DNA hypomethylation has been proposed to have an important impact on tumor biology through the generation of chromosomal instability, reactivation of transposable elements, and loss of imprinting (Esteller, 2008). Thus, a negative correlation between genomic hypomethylation and survival of CM patients could have been expected. Instead, the authors found that hypomethylation of LINE-1 elements at CpG2 or CpG3 sites was associated with a significantly better OS, as demonstrated by Kaplan-Meier analysis and log-rank test. The positive prognostic value of LINE-1 hypomethylation the authors have identified in CM is in sharp contrast with data most recently obtained in colon and ovarian cancer patients, in which LINE-1 hypomethylation in neoplastic tissues was associated with a poorer prognosis (Ogino et al., 2008; Pattamadilok et al., 2008). Since CM behaves differently from other solid tumors, the underlying biological effect(s) of LINE-1 hypomethylation on patients' outcome could depend on the tumor histotype. Nevertheless, it should be emphasized that our findings are generated from patients in the same clinico-pathological stage of disease, while the studies on ovarian and colon cancer were conducted on the heterogeneous patients population as a whole, and did not investigate the prognostic potential of LINE-1 methylation in specific clinically defined stages of disease.

The mechanism(s) through which LINE-1 hypomethylation affects survival of CM patients remains to be fully explored; however, some speculation can be made, based on recent data in the literature. Tellez et al (Tellez et al., 2009) have demonstrated that higher levels of LINE-1 methylation correlate with an increased number of aberrantly hypermethylated tumor suppressor genes (TSG) in cultured melanoma cell lines. Thus, epigenetic inactivation of TSG might account for more aggressive disease the authors have observed in patients with elevated LINE-1 methylation in their neoplastic cells. This notion is in accordance with initial studies reporting a negative association between survival and the presence of hypermethylated ER-α, RASSF1A, RAR-β2, or MINT31 DNA in neoplastic tissues or sera of stage III/IV CM patients (Mori et al., 2006; Mori et al., 2005; Tanemura et al., 2009). On the other hand, hypomethylation, and consequent transcriptional activation, of LINE-1 elements might per se reduce the tumorigenic potential of neoplastic cells by triggering apoptosis and a senescence-like state through the activity of the second open reading frame of LINE-/(Wallace et al., 2008). Furthermore, genomic DNA hypomethylation has been associated with the de novo expression of tumor associated antigens belonging to the Cancer Testis Antigen (CTA) class by neoplastic cells of different histotype (De Smet et al., 1996; Sigalotti et al., 2002; Woloszynska-Read et al., 2008), and the authors have recently identified a significant correlation between a hypomethylated status of LINE-1 elements and increased presence, levels and total number of CTA concomitantly expressed in short-term cultures of CM cells (see example 2). Thus, it is intriguing to speculate that a better immune recognition of LINE-1 hypomethylated CM cells might contribute to the improved survival of these patients.

Irrespective of the underlying biological mechanism(s) triggered by LINE-1 hypomethylation, the prognostic value of LINE-1 methylation here identified for stage IIIC CM patients bears several important practical clinical implications. Among these, the goal to provide CM patients with improved clinico-pathological sub-stage and/or follow-up-procedures would be enhanced using LINE-1 methylation status, and these findings might be used to select and/or stratify patients for adjuvant treatment based on the methylation level of LINE-1 in their tumors. In addition, the significant positive prognosis of LINE-1 hypomethylated patients should prompt the incorporation of this in new studies aimed at understanding whether pharmacologic DNA hypomethylation (Sigalotti et al., 2007) could be regarded as a feasible chemoprevention approach in the initial phases of disease and/or in patients at high-risk of disease recurrence.

The authors' present findings will be further investigated in prospective multicenter studies in which the prognostic significance and the predictive value for different treatments of CM will be validated. Providing further support to our data will undoubtedly add the evaluation of LINE-1 methylation into the routine clinico-pathological ascertainment of CM patients, helping to personalize their comprehensive clinical management

Hypomethylation (i.e., methylation below median) at CpG2 and CpG3 sites significantly associated with improved prognosis of CM, CpG3 showing the strongest association. Patients with hypomethylated CpG3 had increased OS (P=0.01, log-rank=6.39) by Kaplan-Meier analysis. Median OS of patients with hypomethylated or hypermethylated CpG3 were 31.9 and 11.5 months, respectively. The 5 year OS for patients with hypomethylated CpG3 was 48% compared to 7% for patients with hypermethylated sequences. Among the variables examined by Cox regression analysis, LINE-1 methylation at CpG2 and CpG3 was the only predictor of OS (Hazard Ratio=2.63, for hypermethylated CpG3; 95% Confidence Interval: 1.21-5.69; P=0.01).

LINE-1 methylation is identified as a molecular marker of prognosis for CM patients in stage IIIC. Evaluation of LINE-1 promises to represent a key tool for driving the most appropriate clinical management of stage III CM patients.

### Example 2

### Methylation Levels of the “Long Interspersed Nucleotide Element-1” Repetitive Sequences Correlate with Presence and Levels of Expression of Cancer Testis Antigens in Cutaneous Melanoma

Cancer Testis Antigens (CTA) represent an important class of immunogenic tumor antigens that are currently utilized as targets in several cancer immunotherapy approaches. The expression of CTA has been shown to depend on the methylation status of their promoters. In view of the therapeutic relevance of CTA expression and of its regulation by promoter methylation, the authors asked the question whether LINE-1 methylation might correlate with CTA expression in CM cells, thus representing a surrogate marker for their presence and levels of expression. To this end, LINE-1 methylation was measured in CM cells by pyrosequencing assays and CTA expression was evaluated by qualitative or quantitative RT-PCR analyses. The correlation between LINE-1 methylation and CTA expression was evaluated through Spearman's rank correlation test.

### Materials and Methods

### LINE-1 Methylation

The extent of methylation of LINE-1 elements was measured in primary cultures of CM cells at the 6^{th} in vitro passage by bisulfite pyrosequencing as reported in example 1.

### Qualitative and Quantitative RT-PCR Analyses

Total RNA was extracted from CM cells at the 6^{th} in vitro passage using the TRIzol reagent (Invitrogen, Milan, Italy) following manufacturer's recommendations. RT-PCR reactions were performed as previously described (Coral et al., 1999). The oligonucleotide primer sequences and gene-specific PCR amplification programs utilized have been defined for MAGE-A1, -A2, -A3, -A4, -A10(De Plaen et al., 1994; Rimoldi et al., 1999), NY-ESO-1 (Jager et al., 1998), SSX-2(Gure et al., 1997). The integrity of each RNA and oligo-dT-synthesized cDNA sample was confirmed by the amplification of the (3-actin housekeeping gene (Coral et al., 1999). Ten μl of each RT-PCR sample were run on a 2% agarose gel and visualized by ethidium bromide staining.

Real-time quantitative RT-PCR analyses were performed as previously described (Calabro et al., 2005). Briefly, total RNA was digested with RNase free DNase (Roche Diagnostics, Milan, Italy) to remove contaminating genomic DNA. Synthesis of cDNA was performed on 1 μg total RNA using MMLV reverse transcriptase (Invitrogen) and random hexamer primers (Promega, Milan, Italy), following manufacturers' instructions. TaqMan quantitative RT-PCR reactions were performed on 10 ng of retrotranscribed total RNA in a final volume of 25 μl TaqMan Universal Master Mix (Applyed Biosystems, Milan, Italy) at95° C. for 10 min, followed by 45 cycles at 95° C. for 15 sec and at 60° C. for 1 min. TaqMan primers/probe sets were as follows: β-actin forward CGAGCGCGGCTACAGCTT (SEQ ID No. 224), β-actin reverse CCTTAATGTCACGCACGATT (SEQ ID No. 225), β-actin probe FAM-ACCACCACGGCCGAGCGG-BHQ1 (SEQ ID No. 226); MAGE-A3 forward TGTCGTCGGAAATTGGCAGTAT (SEQ ID No. 227), MAGE-A3 reverse CAAAGACCAGCTGCAAGGAACT (SEQ ID No. 228), MAGE-A3 probe FAM-TCTTTCCTGTGATCTTC-MGB (SEQ ID No. 229). Measurement of gene expression was performed utilizing the ABI PRISM 7000 Sequence Detection System (Applyed Biosystems) and the copy number of MAGE-A3 and of the reference gene β-actin was established in each sample by extrapolation of the standard curve. The number of MAGE-A3 cDNA molecules in each sample was then normalized to the number of cDNA molecules of β-actin.

### Statistical Analyses

The correlation between LINE-1 methylation at each CpG site and the number of expressed CTA or the quantitative levels of expression of MAGE-A3 was evaluated through Spearman's rank correlation test. P values<0.05 were considered to be statistically significant.

### Results

Results demonstrate a significant inverse association of LINE-1 methylation with both number of CTA concomitantly expressed (FIG. 4) and level of expression of the CTA MAGE-A3 (FIG. 5).

### Example 3

### Whole-Genome Methylation Profiles, and Derived Methylation Signatures, Predict Survival of Melanoma Patients

### Introduction

Cutaneous melanoma (CM) is a very aggressive neoplasm of growing incidence and mortality rates in industrialized countries, and the leading cause of skin cancer-related deaths worldwide (MacKie et al., 2009). Surgery, in early phases of disease has curative potential for patients; for advanced CM conventional therapies have failed to prolong survival (Bhatia et al., 2009). At present, the best predictor of 5-year survival is the clinico-pathological stage of disease, which defines overall survival (OS) rates ranging from 95% to 7% for stage I to IV patients, respectively (Balch et al., 2001; Balch et al., 2009). However, within the same clinico-pathological stage category, patients often behave radically differently, and the current lack of prognostic molecular markers impairs our ability to identify CM patients with highly aggressive as opposed to more indolentcourses of disease (Jennings and Murphy, 2009).

Methylation of genomic DNA in mammals occurs at SC-position of the cytosine in the context of CpG dinucleotides, and results in gene silencing through different mechanisms (Sigalotti et al., 2010). Recent studies have demonstrated that alterations in genomic DNA methylation patterns represent a hallmark of cancer cells, being proposed to actively contribute to cancer development and progression mainly through inactivation of tumor suppressor genes by aberrant promoter hypermethylation (Esteller, 2008). In particular, epigenetic alterations are emerging as important players in CM tumorigenic process, as demonstrated by the continuously growing list of genes that are transcriptionally inactivated by aberrant DNA hypermethylation in CM, and which potentially affect key cellular pathways such as cellular proliferation/differentiation, apoptosis, DNA repair, extracellular matrix interaction, signaling, metastatization and immune recognition (Sigalotti et al., 2010). The increasing role of aberrant methylation in CM biology strongly suggests for the opportunity to test methylation markers as potential indicators of disease prognosis. Along this line, preliminary investigations are suggesting a possible prognostic role of the methylation status of selected genes in CM patients. Indeed, Lahtz et al have most recently evaluated a total of 230 CM patients at stage 0 to IV of disease, demonstrating PTEN methylation to be an independent negative prognostic factor, though it did not outperform the strongest traditional markers of tumor thickness and ulceration (Lahtz et al., 2010). Similarly, methylation of the tumor suppressor gene TSLC1 was found to be significantly increased in advancing tumor stage and to associate with a reduced disease-related survival of CM patients (You et al., 2010), and the methylation of the methylated in tumors (MINT) locus MINT31 has been recently shown to be significantly associated with advancing clinical stage in 107 stage I to IV CM patients, and to predict improved disease-free and overall survival in the 25 analyzed stage III patients (Tanemura et al., 2009).

In line with the above reported information, initial studies have also investigated the methylation status of selected tumor suppressor genes in sera of CM patients. Among the genes more frequently methylated in CM, a negative correlation between presence of circulating methylated DNA for RASSF1A and ER-a and survival of CM patients receiving biochemotherapy was found, suggesting for the potential usefulness of soluble methylation markers as prognostic factors in this malignancy (Mori et al., 2006; Mori et al., 2005).

Despite these promising initial results, the studies so far available have several limitations, including: i) investigation of only single/few genes; ii) reduced number of patients evaluated; iii) rarely investigated the prognostic significance of methylation markers in patients at the same stage of disease.

Furthermore, to the best of our knowledge, no studies have investigated the influence of the genome-wide gene methylation profiles on CM prognosis. Accordingly, the authors investigated whether whole-genome methylation profiles may account for the differing survival patterns of CM patients of identical clinico-pathological stage of disease. The study was conducted on a series of 59 consecutive stage III CM patients for whom the autologous short-term cell cultures were available. The latter were analyzed early during in vitro passage, and utilized instead of tumor tissues to overcome possible alterations in the evaluation of genomic methylation profiles due to the unavoidable presence of contaminating normal cells.

Results demonstrated that whole-genome methylation profiling is a powerful tool to identify CM patients with a significantly different prognosis, and that methylation signatures of 17 to 98 genes can be utilized to assign CM patients to distinct prognostic groups. These findings demonstrate that evaluation of whole-genome-defined methylation signatures may greatly help in guiding the daily clinical management of CM patients, and provide a strong rationale for the development of a large prospective validation study.

### Materials and Methods

### Patients and Cell Cultures

Short-term cell cultures were established from metastatic lesions removed surgically from consecutive CM patients referred to the National Cancer Institute of Aviano (Italy) for stage III surgery from 1991 to 2007, as previously described (Altomonte et al., 1993). Autologous tumor cell cultures were successfully established from 30% of patients. The micrometastatic nature of lymph-node tumor tissues from AJCC stage IIIA patients precluded their use for cell culture generation. Thus, the planned studies were conducted on a total of 59 available short-term cultures, identified as having been generated from CM patients classified as AJCC stage IIIB or IIIC. Short-term CM cell cultures were grown in RPMI 1640 Medium (Biochrome KG, Berlin, Germany) supplemented with 20% heat-inactivated fetal calf serum (Biochrome KG) and 2 mM L-glutamine (Biochrome KG). To minimize alterations potentially arising with extended in vitro culturing, all cell cultures were utilized for molecular assays at the 6^{th} ex vivo passage.

### Whole Genome Methylation Profiling

Genomic DNA was extracted from short-term cultures of CM cells by proteinase K treatment followed by standard phenol/chloroform extraction and ethanol precipitation (Fratta et al., 2010). The 59 samples under analysis were evaluated for genome-wide promoter methylation using the Illumina Infinium HumanMethylation27 Bead array (Illumina Inc, San Diego, Calif.), which allows the interrogation of 27,578 CpG dinucleotides, covering 14,495 genes. The assay is based on the conversion of unmethylated C-nucleotides into U(T) nucleotides by the bisulfite treatment, leaving unaltered methylated ones. Accordingly, the EZ DNA methylation kit (Zymo Research, Orange, Calif., USA) was used for bisulfite conversion of 500 ng of genomic DNA, and the remaining assay steps were performed as per Illumina protocol. Briefly, the bisulfite-modified genomic DNA was whole-genome amplified, enzymatically fragmented, precipitated, resuspended, and hybridized overnight to locus-specific oligonucleotide primers on the BeadArray. Each interrogated locus is represented by specific oligomers linked to two bead types: one representing the sequence for the methylated DNA (M) and the other for unmethylated DNA (U). After hybridization, the C or T nucleotides were detected on M- or U-beads, respectively, by single-base primer extension. The arrays were then imaged using a BeadArray™ Reader. Image processing and intensity data extraction were performed according to Illumina's instructions. The methylation status of a specifc CpG site is calculated from the intensity of the M- and U-beads, as the ratio of fluorescent signals: β=Max(M,0)/[Max(M,0)+Max(U,0)]. DNA methylation β values are continuous variables between 0 (no methylation) and 1 (completely methylated) representing the ratio of combined locus intensity.

### Gene Expression Profiling

Total cellular RNA was purified from short-term cultures of CM cells by TRIzol reagent (Invitrogen, Milan, Italy) following manufacturer's recommendations. Gene expression profiling was conducted on Illumina human WG-6 version 3 arrays using standard Illumina protocols.

### Statistical Analysis

All statistical analyses were performed using the R, version 2.11.1, statistical environment (http://www.R-project.org) and bioconductor2.6 packages (http://www.bioconductor.org). The authors used the methylumi package for importing and pre-processing the methylation data, the package lumi (Du et al., 2008) for importing and pre-processing the gene expression data, the survival package for Kaplan-Meier estimates and the pamr package for the shrunken centroid supervised analysis. In brief, the methylation data exported from the Illumina Beadstudio software were imported in R, checked for quality and normalized using the methylumi bioconductor package. K-means partitioning clustering was used to divide the transformed data into respectively 2 and 3 groups. The survival times of each of the ⅔ groups were evaluated using a Kaplan-Meier analysis. The characteristics including age, gender, primary tumor localization, Breslow thickness, Clark level, and ulceration of the primary tumor, number of lymph nodes involved, and pre-operative serum LDH values were also examined. Survival time was calculated in months from the date of stage III or IIIC diagnosis until the date of death. According with the specific goals of the analysis, the authors did not classify the deaths considering their cause. Patients were censored at the last follow-up date or the last date the patient was last known to be alive. Median survival duration was determined by the Kaplan-Meier method. Cumulative survival was evaluated using the log-rank test. P values were two sided and values<0.05 were considered to be statistically significant. Methylation patterns (signatures) related to the discovered groups were determined using the nearest shrunken centroid classification algorithm (PAM). As the algorithm requires a threshold to balance between the number of sample correctly classified and the subset of features representing the methylation patterns, the authors determined this threshold by cross-validation as recommended by the inventors of the algorithm (Tibshirani et al., 2002).

### Results

### Patients

The study was conducted on CM patients who underwent radical lymph node dissection for stage III disease at the Centro di Riferimento Oncologico National Cancer Institute between 1991 and 2007. Patients diagnosed with a stage III disease, and for whom a short-term cell culture had been successfully generated from the surgically removed autologous neoplastic tissue, were included in the study. Table 8 summarizes the 59 patients under study and their clinico-pathologic characteristics at presentation.

TABLE 8
Characteristics of the 59 AJCC stage III melanoma patients

| Variable | n. patients | % |
|---|---|---|
| Sub-stage | | |
| IIIB | 13 | 22 |
| IIIC | 45 | 76 |
| NA* | 1 | 2 |
| Age, years | | |
| Median | 52 | |
| Range | 27-84 | |
| Gender | | |
| Male | 35 | 59 |
| Female | 24 | 41 |
| Localization of primary tumor | | |
| extremities | 21 | 36 |
| trunk | 31 | 52 |
| head & neck | 4 | 7 |
| NA | 3 | 5 |
| Breslow thickness of primary tumor | | |
| ≦2.0 mm | 20 | 34 |
| >2.0 mm | 29 | 49 |
| NA | 10 | 17 |
| Clark level of primary tumor | | |
| 1-3 | 19 | 32 |
| 4-5 | 31 | 53 |
| NA | 9 | 15 |
| Ulceration of primary tumor | | |
| No | 20 | 34 |
| Yes | 36 | 61 |
| NA | 3 | 5 |
| N. lymph nodes involved | | |
| 1 | 19 | 32 |
| >1 | 39 | 66 |
| NA | 1 | 2 |
| LDH | | |
| Low_{†} | 37 | 63 |
| High | 19 | 32 |
| NA | 3 | 5 |

*NA, not available
_{†}low LDH is established as LDH values ≦ 0.8 times the upper limit of normal, high LDH is established as LDH values > 0.8 times the upper limit of normal

Unsupervised Analysis of Whole-Genome Methylation Profiles Identifies CM Patients Subgroups with Different Prognosis

In light of the likely influence of altered genome-wide methylation profiles on CM biology, the authors sought to investigate whether distinct methylation patterns might associate with a different clinical outcome of CM patients.

Genome-wide gene methylation profiles were evaluated in the 59 short-term CM cell cultures under study using the Illumina HumanMethylation27 Bead-Chip whole-genome assay, which interrogates the methylation status of 27,578 CpG sites, corresponding to 14,495 genes. Patients were then grouped according to their whole genome methylation profile by applying a k-means clustering, which is a non hierarchical unsupervised method that allows partitioning of data into a predetermined number (k) of groups (FIG. 6). K-means clustering algorithm was applied by requiring a separation of all stage III CM patients under analysis into 2 or 3 subgroups (FIG. 6). Accordingly, the classification split the population in either 2 subgroups (group K2-1 and K2-2), consisting of 35 and 24 patients, respectively, or in 3 subgroups (group K3-1, K3-2, K3-3), counting 27, 24 and 8 patients, respectively (FIG. 6).

The prognostic relevance of the genome-wide methylation profile-based classification was assessed by evaluating the obtained subgroups of patients for overall survival (OS) through Kaplan-Meier analysis. Results demonstrated a significant survival advantage for patients classified as K2-1 as compared to those belonging to group K2-2 (P=0.022, log-rank=5.2) (FIG. 6A). The statistically significant difference in OS among groups was maintained also when splitting the population in 3 classes (P=0.047, log-rank=6.1) (FIG. 6B). In line with these data, an increased median OS was observed for patients classified as K2-1 (31.5 months) as compared to patients in the K2-2 group (14.8 months) (Table 9). Similarly, patients in the K3-1 class had a superior median OS (31.9 months) as compared to K3-3 (18.8 months) and K3-2 (14.8 months) (Table 9). Accordingly, the 5 year OS of patients classified as K2-1 and K2-2 was 41.8% and 5.6%, respectively, while that of patients classified as K3-1, K3-3 and K3-2 was 43.7%, 37.5%, and 5.6%, respectively (Table 9).

TABLE 9
OS of stage III CM patients according
to genome-widemethylation profiles

| stage | group* | Median OS (95% CI)^{†} | | 5 year OS (%) |
|---|---|---|---|---|
| III aII | K2-1 | 31.5 | (13.5-NA‡) | 41.8 |
| | K2-2 | 14.8 | (10.4-35.4) | 5.6 |
| III aII | K3-1 | 31.9 | (15.3-NA) | 43.7 |
| | K3-2 | 14.8 | (10.4-35.4) | 5.6 |
| | K3-3 | 18.8 | (11.5-NA) | 37.5 |
| IIIc | 3C-K2-1 | 31.5 | (13.12-NA) | 41.2 |
| | 3C-K2-2 | 10.4 | (5.29-NA) | 0 |
| IIIc | 3C-K3-1 | 11.0 | (7.3-NA) | 0 |
| | 3C-K3-2 | 31.5 | (12.5-NA) | 39.9 |
| | 3C-K3-3 | 24.5 | (11.5-NA) | 42.9 |

*Patients were divided by the k-means clustering algorithm in either 2 or 3 groups, according to their genome-wide methylation profile;
^{†}Survival functions were calculated by the Kaplan-Meier method. Data are reported as Median OS in months, together with the corresponding 95% Confidence Intervals (CI).
‡NA, not available.

In light of the above reported prognostic significance of genome-wide methylation profiles in stage III CM patients, the authors asked the question whether methylation profiling could allow to identify different survival classes also in sub-stage IIIC patients, which are characterized by highly homogeneous clinico-pathological characteristics. Thus, the same analytical procedures employed for the whole population of stage III patients were applied to the subgroup of 45 stage IIIC patients. K-means classification split the population in either 2 subgroups (group 3C-K2-1 and 3C-K2-2), counting of 33 and 12 patients, respectively, or in 3 subgroups (group 3C-K3-1, 3C-K3-2, 3C-K3-3), including 11, 27 and 7 patients, respectively (FIG. 6). Kaplan-Meier analysis demonstrated a significant survival advantage for patients classified as 3C-K2-1 as compared to group 3C-K2-2 (P=0.001, log-rank=10.2) (FIG. 6C). Similarly, a significant difference in OS was also observed among groups 3C-K3-3, 3C-K3-2, and 3C-K3-1 (P=0.021, log-rank=7.8) (FIG. 6D). An increased median OS was observed for patients classified as 3C-K2-1 (31.5 months) as compared to patients in the 3C-K2-2 group (10.4 months) (Table 9). Similarly, patients in the 3C-K3-2 class had a superior median OS (31.5 months) as compared to 3C-K3-3 (24.5 months) and 3C-K3-1 (11 months) (Table 9). Accordingly, the 5 year OS of patients classified as 3C-K2-1 and 3C-K2-2 was 41.2% and 0%, respectively, while that of patients classified as 3C-K3-3, 3C-K3-2 and 3C-K3-1 was 42.9%, 39.9%, and 0%, respectively (Table 9).

Laying on the above reported information, Cox univariate analysis was carried out to identify patient characteristics and clinico-pathologic factors that predicted survival. Among all examined factors, including age, gender, localization of primary tumor, Breslow thickness, Clark level and ulceration of primary tumor, number of lymph nodes involved, and level of pre-operative LDH, only the classes defined by k-means algorithm on the genome-wide methylation profiles were associated with statistically significant differences in OS, both when evaluating the entire population of stage III CM patients (HR=1.99 for group K2-2 vs. K2-1; 95% CI: 1.09-3.64; P=0.025), and the sole substage IIIC patients (HR=3.2 for group 3C-K2-2 vs. 3C-K2-1; 95% CI: 1.51-6.79; 0.0023) (Table 10).

TABLE 10
Cox analysis of the influence of genome-wide methylation
profile on OS of stage III CM patients

| stage | group* | HR^{†} | 95% CI; P value |
|---|---|---|---|
| III | K2-1 | 1^{§} | |
| | K2-2 | 1.99 | 1.09-3.64; 0.025 |
| IIIC | 3C-K2-1 | 1 | |
| | 3C-K2-2 | 3.20 | 1.51-6.79; 0.0023 |

*Patients were divided by the k-means clustering algorithm in 2 groups, according to their genome-wide methylation profile;
^{†}Cox proportional hazard method was used to examine the effect of methylation-based k-means clustering on OS. Results were presented as Hazard Ratios (HR) with corresponding 95% Confidence Intervals (CI);
^{§}set as reference.

Supervised Analysis of Whole-Genome Methylation Profiles by Shrunken Centroids Identifies the Methylation Signature of CM Patients Subgroups with Different Prognosis

To define the methylation signature, that is the minimal number of methylation markers succinctly characterizing each of the CM patient prognostic group defined by the unsupervised clustering analysis, the authors have applied the “nearest shrunken centroid” algorithm (Tibshirani et al., 2002) (FIG. 7, 8).

Using this approach the authors have identified different methylation signatures able to correctly classify patients into the previously defined prognostic classes, with acceptable error rates (FIG. 7, 8). Indeed, methylation signature A, consisting of 24 genes, had an overall error rate of 0.05 and classified stage III patients into classes K2-1 and K2-2 with a total of 3 errors, while methylation signature B, including 98 genes, had an overall error rate of 0.034 and classified stage III patients into classes K3-1, K3-2, and K3-3 with a total of 2 errors (Table 3, FIG. 7). The same approach conducted on sub-stage IIIC patients led to the identification of a methylation signature of 16 genes (signature C) able to sort stage 111C patients into groups 3C-K2-1 and 3C-K2-2 without any misinterpretation, and a methylation signature of 47 genes (signature D) able to sort stage IIIC patients into groups 3C-K3-1, 3C-K3-2, and 3C-K3-3 with a total of 1 error (overall error rate of 0.022) (Table 3, FIG. 8).

The initial analysis was further refined in the attempt to identify methylation signatures that were likely to be directly associated to a biologic effect through the transcriptional regulation of the respective genes. To this end, a Spearman's correlation analysis was conducted comparing genome-wide methylation data and gene expression profiles obtained from neoplastic cells of the stage III CM patients under study. This analysis yielded a set of 820 methylation probes which methylation status was significantly correlated (p<0.05) with the expression of the respective gene. By restricting the application of the “nearest shrunken centroid” algorithm to this sub-set of methylation signals, the authors were able to identify methylation signatures capable to correctly classify stage IIIC patients into the previously defined prognostic classes (Table 3, FIG. 9). Of these, a 51 gene methylation signature (signature E) was able to assign stage IIIC patients to groups 3C-K2-1 or 3C-K2-2, committing only one error (overall error rate 0.022), while a 73 gene methylation signature (signature F) was able to sort stage IIIC patients into groups 3C-K3-1, 3C-K3-2, and 3C-K3-3 without any misinterpretation (Table 3, FIG. 9). When, among the genes having a significant (p<0.05) association between expression and methylation status, only those with a coefficient of correlation Rho<−0.4 were taken into account, an additional signature of 35 genes (signature G) was defined, which was able to assign stage IIIC patients to groups 3C-K2-1 or 3C-K2-2, committing only one error (overall error rate 0.022) (Table 3, FIG. 10).

Whole genome methylation profiles identified classes with significantly different OS in stage III CM patients. As an example, stage IIIC patients with a “favorable” methylation profile had increased OS (P=0.001, log-rank=10.2) by Kaplan-Meier analysis. Median OS of stage IIIC patients with a “favorable” vs “unfavorable” methylation profile were 31.5 and 10.4 months, respectively. The 5 year OS for stage IIIC patients with a “favorable” methylation profile was 41.2% as compared to 0% for patients with an “unfavorable” methylation profile. Among the variables examined by Cox regression analysis, classification defined by methylation profile was the only predictor of OS (Hazard Ratio=3.2, for “unfavorable” methylation profile; 95% Confidence Interval: 1.51-6.79; P=0.002). Different methylation signatures, consisting of 17 to 98 genes, able to correctly assign (overall error rate<0.05) stage III or IIIC patients to distinct methylation-defined prognostic groups, were identified.

Discrete whole-genome methylation signatures have been identified as molecular markers of prognosis for CM patients in stage III. Their use in the daily practice will drive the most appropriate clinical management of stage III CM patients.

### Example 4

### Prognostic Significance of the Methylation Status of Single Genes Taken from Whole-Genome Defined Prognostic Methylation Signatures

In light of the prognostic relevance of whole-genome methylation profiles that the authors have identified (see example 3 above), the authors have addressed the question whether specific genes taken from the prognostic methylation signatures may retain a prognostic information. To this end, the authors selected genes ALOX12B, CTAG2, FGF4, IGLL1, OCIAD2, PAGES, S100A9, SLC6A11, SLC6A18, and TUB from signature E and G as playing a major contribution in the classification tasks. The 45 stage IIIC patients under study were scored accordingly to the methylation of each gene in their tumor cells being<33%, between 33% and 66%, and >66%, or < and >50%. The impact on patients' survival of ALOX12B, CTAG2, FGF4, IGLL1, OCIAD2, PAGES, S100A9, SLC6A11, SLC6A18, and TUB methylation levels was evaluated by Kaplan-Meier analysis and Log-rank test, showing a significantly improved survival for patients with unmethylated vs methylated genes (Table 11, FIG. 11). Similar results were found for other tested genes, including: BATF, BGN, CRHR1, CSAG1, CTSK, DENND2D, FLJ33860, GIBS, GRM4, LY96, MAGEAL MAGEA10, MGC35206, MLSTD1, SLC18A2, SORBS2, and TRIM40 (data not shown).

TABLE 11
OS of stage IIIC CM patients according to methylation
status of selected signature genes

| | extent | | | |
|---|---|---|---|---|
| gene | methylation* | Median OS (95% CI)^{†} | | 5 year OS (%) |
| FGF4 | <33% | 35.2 | (24.5-NA‡) | 49 |
| | ≧33%, <66% | 11.5 | (6.8-NA) | 0 |
| | ≧66% | 9.8 | (5.3-NA) | 8 |
| CTAG2 | <33% | NA | (31.9-NA) | 58 |
| | ≧33%, <66% | 15.3 | (4.5-NA) | 15 |
| | ≧66% | 11.3 | (7.3-NA) | 0 |
| OCIAD2 | <33% | 27.0 | (11.5-NA) | 44 |
| | ≧33%, <66% | 18.1 | (11.1-NA) | 28 |
| | ≧66% | 9.4 | (5.0 -NA) | 0 |
| PAGE5 | <33% | 73.9 | (24.5-NA) | 58 |
| | ≧33%, <66% | NA | (15.3-NA) | 53 |
| | ≧66% | 11.1 | (7.3-24.5) | 12 |
| S100A9 | <33% | 35.2 | (12.5-NA) | 48 |
| | ≧33%, <66% | 20.9 | (11.5-NA) | 24 |
| | ≧66% | 11.1 | (7.3-NA) | 0 |
| SLC6A18 | <33% | 31.5 | (12.4-NA) | 41 |
| | ≧33%, <66% | 9.8 | (5.3-NA) | 0 |
| | ≧66% | 11.5 | (11-NA) | 0 |
| ALOX12B | <33% | 24.5 | (13.1-NA) | 39 |
| | ≧50% | 8.5 | (5.3-NA) | 0 |
| IGLL1 | <33% | 28 | (13.1-NA) | 40 |
| | ≧50% | 11 | (5.3-NA) | 0 |
| SLC6A11 | <33% | 31.5 | (15.3-NA) | 40 |
| | ≧50% | 9.8 | (7.3-NA) | 0 |
| TUB | <33% | 31.5 | (13.1-NA) | 41 |
| | ≧50% | 10.4 | (5.3-NA) | 0 |

*Patients were divided according to the % of methylation of genes selected from prognostic methylation signatures in their tumor cells being <33%, between 33% and 66%, and >66%, or < and ≧50%.
^{†}Survival functions were calculated by the Kaplan-Meier method. Data are reported as Median OS in months, together with the corresponding 95% Confidence Intervals (CI).
‡NA, not available.

### Example 5

### Prognostic Utility of Methylation Scores Defined by Whole-Genome Methylation Profiles in Melanoma

In light of the prognostic relevance of whole-genome methylation profiles that the authors have identified (see example 3 above), the authors have addressed the question whether simple scores summarizing the whole genome methylation profiles could retain a prognostic information. To this end, the genome-wide methylation profile of tumor cells from each patient (defined by Illumina Infinium HumanMethylation27 Bead array, see example 3 above) was summarized with a “methylation score” as follows: methylation for each gene among the patients was standardized by the Z score method, calculated by (X−μ)/σ where X stands for methylation data of each gene in each sample, μstands for mean of methylation of each gene among all samples, and σstands for the standard deviation. Each patient was then assigned a “methylation score” consisting of the average of Z scores for all genes. The 45 stage IIIC patients under study were divided accordingly to the “methylation score” of their tumor cells being below or above the median value of the patients' population. The impact on patients' survival of the whole genome methylation profiling-derived “methylation score” was evaluated by Kaplan-Meier analysis, Log-rank test and Cox regression analysis, showing a significantly improved survival for patients with low vs high “methylation score” (Table 12, FIG. 13).

TABLE 12
OS of stage IIIC CM patients according to whole genome methylation
profiling-derived “methylation score”

| methylation | | 5 year | | |
|---|---|---|---|---|
| score* | Median OS (95% CI)^{†} | OS (%) | HR^{‡} | 95% CI; P value |
| <median | 31.5 (13.1-NA^{§}) | 44 | 1** | |
| ≧median | 12.5 (9.8-33.5) | 17 | 2.15 | 1.05-4.44; 0.04 |

*Patients were divided according to the “methylation score” being < or ≧ the median “methylation score” measured in the examined patients' population;
^{†}Survival functions were calculated by the Kaplan-Meier method. Data are reported as Median OS in months, together with the corresponding 95% Confidence Intervals (CI);
^{‡}Cox proportional hazard method was used to examine the effect of LINE-1 methylation on OS. Results were presented as Hazard Ratios (HR) with corresponding 95% Confidence Intervals (CI);
^{§}NA, not available;
**set as reference.

### Example 6

### Prognostic Significance of Combined Evaluation of LINE-1 Methylation Levels and Multigene Methylation Signatures in Stage III Cutaneous Melanoma Patients

In light of the prognostic relevance of LINE-1 methylation status and of whole-genome methylation profiles that the authors have identified (see examples 1, 3 above), the authors have addressed the question whether combining these two markers would result in an additional prognostic information. To this end, 59 stage III melanoma patients were scored according to their whole genome methylation profile as K2-1 or k2-2 (stage III patients) or S3-K2-1 or S3-K2-2 (substage IIIC patients), and as having hypomethylated or hypermethylated LINE-1 sequences, as detailed in examples 1 and 3. The impact on patients' survival of the combined evaluation of the 2 markers was evaluated by Kaplan-Meier analysis and Log-rank test (FIG. 12, Table 13). Results demonstrated that the combined use of the 2 markers allowed to further discriminate survival groups within the population under study (P<0.05).

TABLE 13
OS of stage III CM patients according to genome-wide
methylation profiles and LINE-1 methylation status

| | | Median OS | | 5 year |
|---|---|---|---|---|
| stage | group* | (95% CI)^{†} | | OS (%) |
| III aII | K2-1, LINE-1 hypomethylated | 73.9 | (31.5-NA‡) | 52.4 |
| | K2-1, LINE-1 hypermethylated | 12.5 | (9.4-NA) | 18.2 |
| | K2-2, LINE-1 hypomethylated | 10.4 | (9.8-NA) | 0 |
| | K2-2, LINE-1 hypermethylated | 18.1 | (11.01-45.6) | 7 |
| IIIC | 3C-K2-1, LINE-1 hypomethylated | 73.8 | (31.5-NA) | 52.5 |
| | 3C-K2-2, LINE-1 hypermethylated | 12.5 | (9.3-NA) | 23.1 |
| | 3C-K2-2, LINE-1 hypomethylated | 6.8 | (3.7-NA) | 0 |
| | 3C-K2-2, LINE-1 hypermethylated | 11.3 | (5.3-NA) | 0 |

*Patients were scored by k-means clustering algorithm as K2-1 or K2-2 (stage III patients) or as S3-K2-1 or S3-K2-2 (sub-stage IIIC patients), according to their genome-wide methylation profile, and as having hypomethylated (< median value) or hypermethylated (≧ median value) LINE-1 sequences. Combined evaluation of this 2 markers resulted in the definition of 4 classes.
^{†}Survival functions were calculated by the Kaplan-Meier method. Data are reported as Median OS in months, together with the corresponding 95% Confidence Intervals (CI).
‡NA, not available

### Example 7

### Analysis of LINE-1 Methylation in Neoplastic Material from Different Sources

DNA was extracted from neoplastic cells and evaluated for LINE-1 methylation at 3 CpG sites (CpG1, 2, 3) by pyrosequencing or for mean LINE-1 methylation by quantitative Methylation-Specific PCR (qMSP).

Neoplastic cells were purified using anti-HMW-MAA antibody-coated magnetic beads from cryopreserved cell suspensions obtained by mechanical and enzymatic dissection of neoplastic lesions surgically removed from CM patients.

Surgically-removed neoplastic tissues from CM patients were flesh-frozen in liquid nitrogen and maintained at −80° C. until use.

Surgically-removed neoplastic tissues from CM patients were formalin-fixed and paraffin-embedded. Ten micrometer sections (total 2 to 6) were cut from paraffin embedded lesions and used for evaluation of LINE-1 methylation.

Neoplastic tissue of different extensions (area 9-100 mm2) was scraped from 15 micrometer sections of formalin-fixed paraffin-embedded CM tissues, and used for evaluation of LINE-1 methylation.

In a further technical validation, neoplastic cells were purified from hematoxylin and eosin stained formalin-fixed paraffin-embedded CM tissues by Laser Capture Microdissection (LCM) using an Arcturus System (Applied Biosystems). Genomic DNA was extracted from LCM tumor cells using the PicoPure DNA kit (Applied Biosystems) and subjected to bisulfite modification by the EZ DNA Methylation-Gold Kit (Zymo Research). The extent of methylation of LINE-1 elements was measured by a qMSP assay using primers: L1M fwd CGCGAGTCGAAGTAGGGC (SEQ ID No. 230) and L1M rev ACCCGATTTTCCAAATACGACCG (SEQ ID No. 231), specific for the methylated sequences; and L1U fwd TGTGTGTGAGTTGAAGTAGGGT (SEQ ID No. 232) and L1U rev ACCCAATTTTCCAAATACAACCATCA (SEQ ID No. 233), specific for the umethylated sequences. SYBR green qMSP reactions were performed with methylated- or unmethylated-specific primer pairs on 2 μl of bisulfite-modified genomic DNA in a final volume of 25 μl 1X Power SYBR green mastermix (Applyed Biosystems) at 95° C. for 10 min, followed by 45 cycles of 15 sec at 95° C. and 1 min at 60° C. Real-time measurement of fluorescence intensity was performed utilizing the ABI PRISM 7000 Sequence Detection System (Applyed Biosystems), and the copy number of methylated or unmethylated sequences for each target gene was established in each sample by extrapolation from the standard curves generated with plasmids carrying fully methylated or unmethylated LINE-1 sequences. The % of methylation was defined as the ratio between methylated molecules and the sum of methylated and unmethylated molecules.

The results are shown in Tables 14 and 15.

TABLE 14
Pyrosequencing analysis of LINE-1 methylation
in neoplastic material from different sources*

| | | | |
|---|---|---|---|
| patient | CpG1 | CpG2 | CpG3 |

neoplastic cells immunosorted from cryopreserved cell suspensions†

| | | | |
|---|---|---|---|
| 403 | 28.4 | 23.2 | 34.0 |
| 390 | 44.3 | 41.1 | 60.1 |
| 554 | 21.9 | 26.2 | 42.0 |
| 578 | 32.2 | 39.7 | 59.5 |
| 475 | 40.3 | 33.9 | 47.3 |
| 514 | 38.6 | 28.7 | 43.9 |

cryopreserved CM neoplastic tissues‡

| | | | |
|---|---|---|---|
| 588 | 68.7 | 51.4 | 67.2 |
| 603 | 29.0 | 25.7 | 39.3 |
| 629 | 48.0 | 37.9 | 58.0 |
| 790 | 50.0 | 32.9 | 47.3 |
| 816 | 42.3 | 34.9 | 50.5 |
| 11766 | 50.6 | 32.0 | 49.9 |
| 2605 | 37.4 | 26.3 | 40.4 |

formalin-fixed paraffin embedded neoplastic lesions§

| patient | n. of sections | CpG1 | CpG2 | CpG3 |
|---|---|---|---|---|
| 11482 | 2 | 40.5 | 25.7 | 38.6 |
| | 4 | 38.6 | 24.8 | 37.6 |
| | 6 | 37.8 | 24.9 | 38.7 |
| 11766 | 2 | 48.4 | 29.2 | 47.3 |
| | 4 | 45.5 | 30.4 | 44.2 |
| | 6 | 45.3 | 30.7 | 44.8 |
| 6432 | 2 | 46.9 | 32.2 | 50.1 |
| | 4 | 48.7 | 36.0 | 53.6 |
| | 6 | 50.1 | 37.6 | 53.2 |
| 2605 | 2 | 42.8 | 29.2 | 43.3 |
| | 4 | 39.5 | 27.3 | 41.6 |
| | 6 | 42.3 | 29.0 | 40.8 |

macro-dissected formalin-fixed paraffin embedded neoplastic lesions††

| patient | area (mm²) | CpG1 | CpG2 | CpG3 |
|---|---|---|---|---|
| 17193 | 9 | 22.8 | 30.2 | 44.2 |
| | 25 | 25.9 | 32.4 | 50.4 |
| | 100 | 28.8 | 28.1 | 47.6 |
| 6432 | 9 | 29.6 | 36.3 | 54.6 |
| | 25 | 23.6 | 35.2 | 55.0 |
| | 100 | 25.3 | 36.0 | 53.6 |

*DNA was extracted from neoplastic cells and evaluated for LINE-1 methylation at 3 CpG sites (CpG1, 2, 3) by pyrosequencing;
†neoplastic cells were purified using anti-HMW-MAA antibody-coated magnetic beads from cryopreserved cell suspensions obtained by mechanical and enzymatic dissection of neoplastic lesions surgically removed from CM patients;
‡surgically-removed neoplastic tissues from CM patients were flesh-frozen in liquid nitrogen and maintained at −80° C. until use;
§surgically-removed neoplastic tissues from CM patients were formalin-fixed and paraffin-embedded. Ten micrometer sections (total 2 to 6) were cut from paraffin embedded lesions and used for evaluation of LINE-1 methylation;
††Neoplastic tissue of different extensions (area 9-100 mm2) was scraped from 15 micrometer sections of formalin-fixed paraffin-embedded CM tissues, and used for evaluation of LINE-1 methylation.

TABLE 15
qMSP analysis of LINE-1 methylation in laser
capture microdissected tumor cells*

| | % LINE1 methlylation | | |
|---|---|---|---|
| patient | cell culture (6p) † | LCM ‡ | Correlation § |
| 629 | 89 | 74 | R = 0.99 (95% CI: 0.86 1.0) |
| 767 | 76 | 67 | P = 0.001 |
| 790 | 48 | 55 | |
| 812 | 22 | 43 | |
| 816 | 58 | 63 | |

*DNA was extracted from neoplastic cells and evaluated for LINE-1 methylation by qMSP.
† autologous neoplastic cell cultures were evaluated at the 6^{th} in vitro passage;
‡ neoplastic cells obtained from formalin-fixed paraffin-embedded CM tissues by Laser Capture Microdissection (LCM);
§ correlation between % LINE-1 values obtained from 6^{th} passage neoplastic cells and autologous LCM neoplastic cells was evaluated by the Pearson's correlation test. Coefficient of correlation is reported with the corresponding 95% Confidence Interval (CI). Reported P value is 2-sided.

### REFERENCES

Altomonte, M., et al. 1993. Cancer Res. 53:3343-3348.
Ausubel, F. M., et al. 1998. Current Protocols in Molecular Biology John Wiley & Sons, NY.
Balch, C. M., et al. 2001. J Clin Oncol. 19:3635-3648.
Balch, C. M., et al. 2009. J Clin Oncol. 27:6199-6206.
Bhatia, S., et al. 2009. Oncology (Williston Park). 23 :488-496.
Calabro, L., et al. 2005. J Cell Physiol. 202:474-477.
Coral, S., et al. 1999. J Immunother. 22:16-24.
Cox, D. R. 1972. J Roy Statist Soc B. 34:187-220.
De Plaen, E., et al. 1994. Immunogenetics. 40:360-369.
De Smet, C., et al. 1996. Proc Natl. Acad. Sci. U.S.A. 93:7149-7153.
Du, P., et al. 2008. Bioinformatics. 24:1547-1548.
Esteller, M. 2008. N Engl J Med. 358:1148-1159.
Fratta, E., et al. 2010. J Cell Physiol. 223:352-358.
Gama-Sosa, M. A., et al. 1983. Nucleic Acids Res. 11:6883-6894.
Gure, A. O., et al. 1997. Int J Cancer. 72:965-971.
Jager, E., et al. 1998. J Exp Med. 187:265-270.
Jennings, L., and G. M. Murphy. 2009. Br J Dermatol. 161:496-503.
Kaplan, E. L., and P. Meier. 1958. J of the Am. Statistical Association. 53:457-481.
Lahtz, C., et al. 2010. J Invest Dermatol. 130:620-622.
MacKie, R. M., et al. 2009. Ann Oncol. 20 Suppl 6:vi1-7.
Mori, T., et al. 2006. Cancer Res. 66:6692-6698.
Mori, T., et al. 2005. J Clin Oncol. 23:9351-9358.
Ogino, S., et al. 2008. J Nall Cancer Inst. 100:1734-1738.
Pattamadilok, J., et al. 2008. Int J Gynecol Cancer. 18:711-717.
Rimoldi, D., et al. 1999. Int J. Cancer. 82:901-907.
Sigalotti, L., et al. 2002. J. Immunother. (1997). 25:16-26.
Sigalotti, L., et al. 2010. J Transl Med. 8:56.
Sigalotti, L., et al. 2007. J Cell Physiol. 212:330-344.
Tanemura, A., et al. 2009. Clin Cancer Res. 15:1801-1807.
Tellez, C. S., et al. 2009. Melanoma Res. 19:146-155.
Tibshirani, R., et al. 2002. Proc Natl Acad Sci USA. 99:6567-6572.
Wallace, N. A., et al. 2008. Gene. 419:75-81.
Woloszynska-Read, A., et al. 2008. Clin. Cancer Res. 14:3283-3290.
Yang, A. S., et al. 2004. Nucleic Acids Res. 32:e38.
You, Y., et al. 2010. Melanoma Res. 20:179-183.

1. An in vitro method of prognosing melanoma within a clinico-pathological stage thereof and/or monitoring the response to a melanoma therapy in a subject and/or to identify a subject to be treated with a DNA hypomethylating agent comprising determining the methylation status of at least one cytosine residue in a methylation relevant region of at least 16 genes selected from the group of: ABCA8, ABCB4, ACOX2, ACSL5, ADM, ADORA2B, ALDH1A3, ALOX12B, AMPH, ANGPT4, ANGPTL3, ANKRD1, ANXA13, ANXA8, APOBEC4, APOD, ARHGDIB, ARHGEF7, ARSG, ATP6V1E2, AVPR1B, AXL, BATF, BGN, BSND, C11orf1, C1orf201, C1orf36, C1orf42, C1orf64, C20orf100, C21orf70, C3orf27, C3orf63, C9orf90, CD1D, CEACAM6, CHMP4A, CLEC4F, COL5A2, CPA3, CRHR1, CSAG1, CST11, CST7, CST9L, CTAG1B, CTAG2, CTNNAL1, CTSK, CX3CR1, CXorf12, CYP2A7, CYP2B6, CYP8B1, DARC, DCLRE1A, DEFB123, DEFB4, DENND2D, DIRC1, DLG5, EMILIN1, EMP1, ERO1L, EVL, FAAH, FAM107B, FAM38B, FANCE, FGF4, FLJ20581, FLJ25421, FLJ32771, FLJ33860, FLJ39501, FMO2, FOXF2, FOXI1, FOXN1, FXYD4, GABRG3, GAGE2, GAS2L2, GJB3, GJB4, GJB5, GLT1D1, GRAMD3, GRM4, GSTT1, GUCA2B, HAO2, HCP5, HLA-DOB, HNMT, IFT140, IGLL1, IL17F, IL28B, IL8RA, IL8RB, KCNG1, KCNK16, KIAA0141, KIAA0367, KRT25C, KRT25D, KRTHB2, KRTHB3, KRTHB4, KRTHB5, LAMC2, LOC124751, LPXN, LRRN5, LY96, MAGEA1, MAGEA10, MAGEA3, MAGEA6, MAGEC2, MGC29671, MGC35206, MGMT, MIA2, MIS12, MLN, MLSTD1, MRVI1, MSMB, MT1G, NALP10, NBL1, NDN, NNMT, NR5A2, OCIAD2, OLFM1, OR12D3, PAGES, PALLD, PCDHAC2, PCOLCE, PDZD4, PGCP, PKHD1, PLA1A, PLD1, PPAT, PPIE, PRDX2, PREPL, PRKCZ, PROCR, PRRX1, PSD4, PTCD2, PVALB, R3HDML, RAB11FIP5, RAC2, RNASE1, RPUSD3, S100A9, SCN4A, SEMG2, SERPINB12, SFTPG, SLAMF9, SLC13A1, SLC18A2, SLC22A8, SLC2A7, SLC44A5, SLC4A7, SLC6A11, SLC6A18, SLCO4A1, SLPI, SORBS2, SPATS1, SPINK5, SSNA1, STK38, SUHW1, TDRD5, TFF3, THRB, TIMP3, TMEM10, TMEPAI, TNFSF13B, TNNI1, TRIM40, TUB, UBE2E1, UNC45B, UNQ9433, WDR20, WFDC12, WFDC8, WNT10B, ZNF324, ZNF532, ZNF558, or a variant thereof, in a biological sample obtained from the subject.

2. The method of claim 1 wherein the genes are: ALOX12B, CRHR1, FGF4, FLJ33860, GJB5, GRM4, IGLL1, MGC35206, PCDHAC2, S100A9, SLC18A2, SLC6A11, SLC6A18, TRIM40, TUB and WNT10B (signature C) and allelic variants thereof.

3. The method of claim 2 wherein, for each of said genes, the methylation relevant regions are comprised in the nucleotide sequences as indicated in Table 3 signature C, or in complementary sequences thereof.

4. The method of claim 3 wherein for each of said genes, the methylation relevant regions consist of: nt 940 to nt 1061 of SEQ ID No. 2, nt 941 to nt 1062 of SEQ ID No. 4, nt 940 to nt 1061 of SEQ ID No. 7, nt 941 to nt 1062 of SEQ ID No. 10, nt 940 to nt 1061 of SEQ ID No. 11, nt 940 to nt 1061 of SEQ ID No. 16, nt 940 to nt 1061 of SEQ ID No. 17, nt 941 to nt 1062 of SEQ ID No. 86, nt 941 to nt 1062 of SEQ ID No. 99, nt 940 to nt 1061 of SEQ ID No. 100, nt 940 to nt 1061 of SEQ ID No. 101, nt 941 to nt 1062 of SEQ ID No. 102, nt 941 to nt 1062 of SEQ ID No. 103, nt 940 to nt 1061 of SEQ ID No. 104, nt 941 to nt 1062 of SEQ ID No. 105, nt 940 to nt 1061 of SEQ ID No. 106, nt 940 to nt 1061 of SEQ ID No. 107, an allelic variant or a complementary sequence thereof.

5. The method of claim 1 wherein the genes are: ANXA8, CRHR1, CST11, CYP2B6, CYP8B1, FLJ33860, FOXI1, FOXN1, FXYD4, GAS2L2, GJB5, GUCA2B, IGLL1, IL8RA, KCNK16, KRTHB2, MGC29671, OR12D3, S100A9, SLPI, TMEM10, TRIM40, TUB, and WFDC8 (signature A) and allelic variants thereof.

6. The method of claim 5 wherein, for each of said genes, the methylation relevant regions are comprised in the nucleotide sequences as indicated in Table 3 signature A, or in complementary sequences thereof.

7. The method of claim 6 wherein for each of said genes, the methylation relevant regions consist of: nt 941 to nt 1062 of SEQ ID No. 1, nt 940 to nt 1061 of SEQ ID No. 2, nt 940 to nt 1061 of SEQ ID No. 3, nt 941 to nt 1062 of SEQ ID No. 4, nt 940 to nt 1061 of SEQ ID No. 5, nt 940 to nt 1061 of SEQ ID No. 6, nt 940 to nt 1061 of SEQ ID No. 7, nt 941 to nt 1062 of SEQ ID No. 8, nt 941 to nt 1062 of SEQ ID No. 9, nt 941 to nt 1062 of SEQ ID No. 10, nt 940 to nt 1061 of SEQ ID No. 11, nt 940 to nt 1061 of SEQ ID No. 12, nt 941 to nt 1062 of SEQ ID No. 13, nt 940 to nt 1061 of SEQ ID No. 14, nt 941 to nt 1062 of SEQ ID No. 15, nt 940 to nt 1061 of SEQ ID No. 16, nt 940 to nt 1061 of SEQ ID No. 17, nt 941 to nt 1062 of SEQ ID No. 18, nt 940 to nt 1061 of SEQ ID No. 19, nt 940 to nt 1061 of SEQ ID No. 20, nt 940 to nt 1061 of SEQ ID No. 21, nt 940 to nt 1061 of SEQ ID No. 22, nt 940 to nt 1061 of SEQ ID No. 23, nt 940 to nt 1061 of SEQ ID No. 24, an allelic variant or a complementary sequence thereof.

8. The method of claim 1 wherein the genes are: ABCA8, ADM, ALDH1A3, ARHGDIB, BGN, C9orf90, COL5A2, CSAG1, CTAG2, CTSK, DENND2D, EMILIN1, GSTT1, HCP5, LPXN, LY96, MAGEA1, MAGEA3, MAGEA6, MAGEC2, NNMT, OCIAD2, PAGES, PCOLCE, PRDX2, RAB11FIP5, RNASE1 and SUHW1 (signature G) and allelic variants thereof.

9. The method of claim 8 wherein, for each of said genes, the methylation relevant regions are comprised in the nucleotide sequences as indicated in Table 3 signature G, or in complementary sequences thereof.

10. The method of claim 9 wherein for each of said genes, the methylation relevant regions consist of: nt 940 to nt 1061 of SEQ ID No. 109, nt 940 to nt 1061 of SEQ ID No. 125, nt 940 to nt 1061 of SEQ ID No. 132, nt 940 to nt 1061 of SEQ ID No. 138, nt 941 to nt 1062 of SEQ ID No. 139, nt 940 to nt 1061 of SEQ ID No. 140, nt 941 to nt 1062 of SEQ ID No. 142, nt 941 to nt 1062 of SEQ ID No. 143, nt 941 to nt 1062 of SEQ ID No. 149, nt 941 to nt 1062 of SEQ ID No. 150, nt 941 to nt 1062 of SEQ ID No. 155, nt 941 to nt 1062 of SEQ ID No. 161, nt 941 to nt 1062 of SEQ ID No. 165, nt 941 to nt 1062 of SEQ ID No. 166, nt 941 to nt 1062 of SEQ ID No. 167, nt 941 to nt 1062 of SEQ ID No. 168, nt 941 to nt 1062 of SEQ ID No. 169, nt 941 to nt 1062 of SEQ ID No. 181, nt 941 to nt 1062 of SEQ ID No. 182, nt 940 to nt 1061 of SEQ ID No. 201, nt 940 to nt 1061 of SEQ ID No. 205, nt 940 to nt 1061 of SEQ ID No. 206, nt 941 to nt 1062 of SEQ ID No. 207, nt 940 to nt 1061 of SEQ ID No. 208, nt 940 to nt 1061 of SEQ ID No. 209, nt 940 to nt 1061 of SEQ ID No. 210, nt 941 to nt 1062 of SEQ ID No. 211, nt 941 to nt 1062 of SEQ ID No. 212, nt 941 to nt 1062 of SEQ ID No. 213, nt 940 to nt 1061 of SEQ ID No. 214, nt 940 to nt 1061 of SEQ ID No. 215, nt 941 to nt 1062 of SEQ ID No. 216, nt 941 to nt 1062 of SEQ ID No. 217, nt 940 to nt 1061 of SEQ ID No. 218, nt 940 to nt 1061 of SEQ ID No. 219, an allelic variant or a complementary sequence thereof.

11. The method of claim 1 wherein the genes are: ACSL5, ALOX12B, ANKRD1, ANXA13, APOBEC4, AVPR1B, Clorf201, CD1D, CEACAM6, CRHR1, CTAG1B, CTAG2, DCLRE1A, DIRC1, FAAH, FGF4, F1125421, FLJ33860, FLJ39501, GABRG3, GJB3, GJB5, GLT1D1, GRM4, HAO2, IFT140, IL28B, KCNK16, LRRN5, MGC35206, MIA2, OR12D3, PCDHAC2, PVALB, S100A9, SLC18A2, SLC4A7, SLC6A11, SLC6A18, STK38, TDRD5, TRIM40, TUB, UBE2E1, UNQ9433 and WNT10B (signature D) or allelic variant thereof.

12. The method of claim 11 wherein, for each of said genes, the methylation relevant regions are comprised in the nucleotide sequences as indicated in Table 3 signature D, or in complementary sequences thereof.

13. The method of claim 12 wherein for each of said genes, the methylation relevant region consist of: nt 940 to nt 1061 of SEQ ID No. 2, nt 940 to nt 1061 of SEQ ID No. 3, nt 940 to nt 1061 of SEQ ID No. 6, nt 940 to nt 1061 of SEQ ID No. 7, nt 941 to nt 1062 of SEQ ID No. 10, nt 940 to nt 1061 of SEQ ID No. 11, nt 940 to nt 1061 of SEQ ID No. 16, nt 940 to nt 1061 of SEQ ID No. 17, nt 940 to nt 1061 of SEQ ID No. 25, nt 940 to nt 1061 of SEQ ID No. 30, nt 940 to nt 1061 of SEQ ID No. 34, nt 940 to nt 1061 of SEQ ID No. 38, nt 940 to nt 1061 of SEQ ID No. 39, nt 940 to nt 1061 of SEQ ID No. 42, nt 940 to nt 1061 of SEQ ID No. 46, nt 940 to nt 1061 of SEQ ID No. 48, nt 940 to nt 1061 of SEQ ID No. 56, nt 940 to nt 1061 of SEQ ID No. 59, nt 940 to nt 1061 of SEQ ID No. 63, nt 941 to nt 1062 of SEQ ID No. 86, nt 940 to nt 1061 of SEQ ID No. 94, nt 941 to nt 1062 of SEQ ID No. 99, nt 940 to nt 1061 of SEQ ID No. 100, nt 940 to nt 1061 of SEQ ID No. 101, nt 941 to nt 1062 of SEQ ID No. 102, nt 941 to nt 1062 of SEQ ID No. 103, nt 940 to nt 1061 of SEQ ID No. 104, nt 941 to nt 1062 of SEQ ID No. 105, nt 940 to nt 1061 of SEQ ID No. 106, nt 940 to nt 1061 of SEQ ID No. 107, nt 940 to nt 1061 of SEQ ID No. 108, nt 940 to nt 1061 of SEQ ID No. 109, nt 940 to nt 1061 of SEQ ID No. 110, nt 940 to nt 1061 of SEQ ID No. 111, nt 940 to nt 1061 of SEQ ID No. 112, nt 941 to nt 1062 of SEQ ID No. 113, nt 941 to nt 1062 of SEQ ID No. 114, nt 941 to nt 1062 of SEQ ID No. 115, nt 940 to nt 1061 of SEQ ID No. 116, nt 941 to nt 1062 of SEQ ID No. 117, nt 941 to nt 1062 of SEQ ID No. 118, nt 940 to nt 1061 of SEQ ID No. 119, nt 941 to nt 1062 of SEQ ID No. 120, nt 940 to nt 1061 of SEQ ID No. 121, nt 941 to nt 1062 of SEQ ID No. 122, nt 941 to nt 1062 of SEQ ID No. 123, nt 940 to nt 1061 of SEQ ID No. 124, an allelic variant or a complementary sequence thereof.

14. The method of claim 1 wherein the genes are: AMPH, ARHGEF7, AXL, BATF, BGN, C20orf100, C21orf70, C9orf90, COL5A2, CSAG1, CST7, CTAG2, CTSK, DENND2D, EMILIN1, EVL, FAM107B, FAM38B, FANCE, FOXF2, HCP5, KCNG1, KIAA0367, LY96, MAGEA1, MAGEA10, MAGEC2, MLSTD1, MT1G, NDN, OCIAD2, PDZD4, PGCP, PPIE, PRKCZ, PROCR, PRRX1, RAC2, RNASE1, SLCO4A1, SORBS2, SPATS1, SSNA1, TFF3, TMEPAI and ZNF324 (signature E) or allelic variants thereof.

15. The method of claim 14 wherein, for each of said genes, the methylation relevant regions are comprised in the nucleotide sequences as indicated in Table 3 signature E, or in complementary sequences thereof.

16. The method of claim 15 wherein for each of said genes, the methylation relevant region consist of: nt 940 to nt 1061 of SEQ ID No. 109, nt 940 to nt 1061 of SEQ ID No. 125, nt 940 to nt 1061 of SEQ ID No. 126, nt 941 to nt 1062 of SEQ ID No. 127, nt 941 to nt 1062 of SEQ ID No. 128, nt 941 to nt 1062 of SEQ ID No. 129, nt 941 to nt 1062 of SEQ ID No. 130, nt 941 to nt 1062 of SEQ ID No. 131, nt 940 to nt 1061 of SEQ ID No. 132, nt 940 to nt 1061 of SEQ ID No. 133, nt 941 to nt 1062 of SEQ ID No. 134, nt 940 to nt 1061 of SEQ ID No. 135, nt 941 to nt 1062 of SEQ ID No. 136, nt 941 to nt 1062 of SEQ ID No. 137, nt 940 to nt 1061 of SEQ ID No. 138, nt 941 to nt 1062 of SEQ ID No. 139, nt 940 to nt 1061 of SEQ ID No. 140, nt 940 to nt 1061 of SEQ ID No. 141, nt 941 to nt 1062 of SEQ ID No. 142, nt 941 to nt 1062 of SEQ ID No. 143, nt 941 to nt 1062 of SEQ ID No. 144, nt 941 to nt 1062 of SEQ ID No. 145, nt 941 to nt 1062 of SEQ ID No. 146, nt 941 to nt 1062 of SEQ ID No. 147, nt 941 to nt 1062 of SEQ ID No. 148, nt 941 to nt 1062 of SEQ ID No. 149, nt 941 to nt 1062 of SEQ ID No. 150, nt 940 to nt 1061 of SEQ ID No. 151, nt 940 to nt 1061 of SEQ ID No. 152, nt 940 to nt 1061 of SEQ ID No. 153, nt 941 to nt 1062 of SEQ ID No. 154, nt 941 to nt 1062 of SEQ ID No. 155, nt 940 to nt 1061 of SEQ ID No. 156, nt 940 to nt 1061 of SEQ ID No. 157, nt 940 to nt 1061 of SEQ ID No. 158, nt 941 to nt 1062 of SEQ ID No. 159, nt 940 to nt 1061 of SEQ ID No. 160, nt 941 to nt 1062 of SEQ ID No. 161, nt 940 to nt 1061 of SEQ ID No. 162, nt 940 to nt 1061 of SEQ ID No. 163, nt 940 to nt 1061 of SEQ ID No. 164, nt 941 to nt 1062 of SEQ ID No. 165, nt 941 to nt 1062 of SEQ ID No. 166, nt 941 to nt 1062 of SEQ ID No. 167, nt 941 to nt 1062 of SEQ ID No. 168, nt 941 to nt 1062 of SEQ ID No. 169, nt 940 to nt 1061 of SEQ ID No. 170, nt 940 to nt 1061 of SEQ ID No. 171, nt 941 to nt 1062 of SEQ ID No. 172, nt 940 to nt 1061 of SEQ ID No. 173, nt 941 to nt 1062 of SEQ ID No. 174, nt 941 to nt 1062 of SEQ ID No. 175, an allelic variant or a complementary sequence thereof.

17. The method of claim 1 wherein the genes are: ABCA8, ABCB4, ACOX2, ADORA2B, AMPH, APOD, ARHGDIB, ARHGEF7, ARSG, ATP6V1E2, AXL, BGN, C11orf1, C20orf100, C21orf70, C9orf90, CHMP4A, COL5A2, CSAG1, CST7, CTAG2, CTNNAL1, CXorf12, DENND2D, EMILIN1, EMP1, ERO1L, EVL, FAM107B, FANCE, FLJ25421, GAGE2, GRAMD3, HCP5, HNMT, KCNG1, KIAA0141, KIAA0367, KRT25C, LY96, MAGEA1, MAGEA10, MAGEA6, MAGEC2, MGMT, MT1G, NBL1, NDN, OLFM1, PDZD4, PGCP, PLA1A, PLD1, PPIE, PROCR, PRRX1, RAC2, RNASE1, RPUSD3, SLAMF9, SLCO4A1, SPATS1, TFF3, TIMP3, TMEPAI, ZNF532 and ZNF558 (signature F) or alleleic variants thereof.

18. The method of claim 17 wherein, for each of said genes, the methylation relevant regions are comprised in the nucleotide sequences as indicated in Table 3 signature F, or in complementary sequences thereof.

19. The method of claim 18 wherein for each of said genes, the methylation relevant region consist of: nt 940 to nt 1061 of SEQ ID No. 25, nt 941 to nt 1062 of SEQ ID No. 61, nt 941 to nt 1062 of SEQ ID No. 80, nt 940 to nt 1061 of SEQ ID No. 109, nt 940 to nt 1061 of SEQ ID No. 125, nt 940 to nt 1061 of SEQ ID No. 126, nt 941 to nt 1062 of SEQ ID No. 127, nt 941 to nt 1062 of SEQ ID No. 128, nt 941 to nt 1062 of SEQ ID No. 129, nt 940 to nt 1061 of SEQ ID No. 132, nt 940 to nt 1061 of SEQ ID No. 133, nt 941 to nt 1062 of SEQ ID No. 134, nt 940 to nt 1061 of SEQ ID No. 135, nt 941 to nt 1062 of SEQ ID No. 137, nt 940 to nt 1061 of SEQ ID No. 138, nt 940 to nt 1061 of SEQ ID No. 140, nt 940 to nt 1061 of SEQ ID No. 141, nt 941 to nt 1062 of SEQ ID No. 142, nt 941 to nt 1062 of SEQ ID No. 143, nt 941 to nt 1062 of SEQ ID No. 144, nt 941 to nt 1062 of SEQ ID No. 145, nt 941 to nt 1062 of SEQ ID No. 146, nt 941 to nt 1062 of SEQ ID No. 147, nt 941 to nt 1062 of SEQ ID No. 148, nt 941 to nt 1062 of SEQ ID No. 149, nt 941 to nt 1062 of SEQ ID No. 150, nt 940 to nt 1061 of SEQ ID No. 151, nt 940 to nt 1061 of SEQ ID No. 152, nt 940 to nt 1061 of SEQ ID No. 153, nt 941 to nt 1062 of SEQ ID No. 155, nt 940 to nt 1061 of SEQ ID No. 156, nt 940 to nt 1061 of SEQ ID No. 157, nt 940 to nt 1061 of SEQ ID No. 158, nt 941 to nt 1062 of SEQ ID No. 159, nt 940 to nt 1061 of SEQ ID No. 160, nt 940 to nt 1061 of SEQ ID No. 162, nt 941 to nt 1062 of SEQ ID No. 165, nt 941 to nt 1062 of SEQ ID No. 166, nt 941 to nt 1062 of SEQ ID No. 167, nt 941 to nt 1062 of SEQ ID No. 168, nt 940 to nt 1061 of SEQ ID No. 170, nt 941 to nt 1062 of SEQ ID No. 172, nt 940 to nt 1061 of SEQ ID No. 173, nt 941 to nt 1062 of SEQ ID No. 175, nt 941 to nt 1062 of SEQ ID No. 176, nt 941 to nt 1062 of SEQ ID No. 177, nt 940 to nt 1061 of SEQ ID No. 178, nt 941 to nt 1062 of SEQ ID No. 179, nt 941 to nt 1062 of SEQ ID No. 180, nt 941 to nt 1062 of SEQ ID No. 181, nt 941 to nt 1062 of SEQ ID No. 182, nt 940 to nt 1061 of SEQ ID No. 183, nt 940 to nt 1061 of SEQ ID No. 184, nt 940 to nt 1061 of SEQ ID No. 185, nt 940 to nt 1061 of SEQ ID No. 186, nt 940 to nt 1061 of SEQ ID No. 187, nt 940 to nt 1061 of SEQ ID No. 188, nt 940 to nt 1061 of SEQ ID No. 189, nt 940 to nt 1061 of SEQ ID No. 190, nt 941 to nt 1062 of SEQ ID No. 191, nt 940 to nt 1061 of SEQ ID No. 192, nt 940 to nt 1061 of SEQ ID No. 193, nt 941 to nt 1062 of SEQ ID No. 194, nt 940 to nt 1061 of SEQ ID No. 195, nt 940 to nt 1061 of SEQ ID No. 196, nt 941 to nt 1062 of SEQ ID No. 197, nt 941 to nt 1062 of SEQ ID No. 198, nt 941 to nt 1062 of SEQ ID No. 199, nt 940 to nt 1061 of SEQ ID No. 200, nt 940 to nt 1061 of SEQ ID No. 201, nt 940 to nt 1061 of SEQ ID No. 202, nt 940 to nt 1061 of SEQ ID No. 203, nt 940 to nt 1061 of SEQ ID No. 204, an allelic variant or a complementary sequence thereof.

20. The method of claim 1 wherein the genes are: ACSL5, ANGPT4, ANGPTL3, ANKRD1, ANXA8, APOBEC4, APOD, ARSG, BSND, Clorf201, Clorf36, Clorf42, Clorf64, C3orf27, C3orf63, CLEC4F, CPA3, CRHR1, CST11, CST9L, CX3CR1, CYP2A7, CYP2B6, CYP8B1, DARC, DCLRE1A, DEFB123, DEFB4, DIRC1, DLG5, FLJ20581, FLJ25421, FLJ32771, FLJ33860, FMO2, FOXI1, FOXN1, FXYD4, GAS2L2, GJB4, GJB5, GUCA2B, HLA-DOB, IFT140, IGLL1, IL17F, IL8RA, IL8RB, KCNK16, KRT25D, KRTHB2, KRTHB3, KRTHB4, KRTHB5, LAMC2, LOC124751, LRRN5, MGC29671, MGC35206, MIA2, MIS12, MLN, MRVI1, MSMB, NALP10, NR5A2, OR12D3, PALLD, PKHD1, PPAT, PREPL, PSD4, PTCD2, R3HDML, S100A9, SCN4A, SEMG2, SERPINB12, SFTPG, SLC13A1, SLC22A8, SLC2A7, SLC44A5, SLPI, SPINK5, STK38, THRB, TMEM10, TNFSF13B, TNNI1, TRIM40, TUB, UBE2E1, UNC45B, WDR20, WFDC12 and WFDC8 (signature B) or allelic variants thereof.

21. The method of claim 20 wherein, for each of said genes, the methylation relevant regions are comprised in the nucleotide sequences as indicated in Table 3 signature B, or in complementary sequences thereof.

22. The method of claim 21 wherein for each of said genes, the methylation relevant regions consist of: nt 941 to nt 1062 of SEQ ID No. 1, nt 940 to nt 1061 of SEQ ID No. 2, nt 940 to nt 1061 of SEQ ID No. 3, nt 941 to nt 1062 of SEQ ID No. 4, nt 940 to nt 1061 of SEQ ID No. 5, nt 940 to nt 1061 of SEQ ID No. 6, nt 940 to nt 1061 of SEQ ID No. 7, nt 941 to nt 1062 of SEQ ID No. 8, nt 941 to nt 1062 of SEQ ID No. 9, nt 941 to nt 1062 of SEQ ID No. 10, nt 940 to nt 1061 of SEQ ID No. 11, nt 940 to nt 1061 of SEQ ID No. 12, nt 941 to nt 1062 of SEQ ID No. 13, nt 940 to nt 1061 of SEQ ID No. 14, nt 941 to nt 1062 of SEQ ID No. 15, nt 940 to nt 1061 of SEQ ID No. 16, nt 940 to nt 1061 of SEQ ID No. 17, nt 941 to nt 1062 of SEQ ID No. 18, nt 940 to nt 1061 of SEQ ID No. 19, nt 940 to nt 1061 of SEQ ID No. 20, nt 940 to nt 1061 of SEQ ID No. 21, nt 940 to nt 1061 of SEQ ID No. 22, nt 940 to nt 1061 of SEQ ID No. 23, nt 940 to nt 1061 of SEQ ID No. 24, nt 940 to nt 1061 of SEQ ID No. 25, nt 941 to nt 1062 of SEQ ID No. 26, nt 941 to nt 1062 of SEQ ID No. 27, nt 941 to nt 1062 of SEQ ID No. 28, nt 940 to nt 1061 of SEQ ID No. 29, nt 940 to nt 1061 of SEQ ID No. 30, nt 940 to nt 1061 of SEQ ID No. 31, nt 940 to nt 1061 of SEQ ID No. 32, nt 940 to nt 1061 of SEQ ID No. 33, nt 940 to nt 1061 of SEQ ID No. 34, nt 940 to nt 1061 of SEQ ID No. 35, nt 940 to nt 1061 of SEQ ID No. 36, nt 940 to nt 1061 of SEQ ID No. 37, nt 940 to nt 1061 of SEQ ID No. 38, nt 940 to nt 1061 of SEQ ID No. 39, nt 941 to nt 1062 of SEQ ID No. 40, nt 940 to nt 1061 of SEQ ID No. 41, nt 940 to nt 1061 of SEQ ID No. 42, nt 940 to nt 1061 of SEQ ID No. 43, nt 940 to nt 1061 of SEQ ID No. 44, nt 941 to nt 1062 of SEQ ID No. 45, nt 940 to nt 1061 of SEQ ID No. 46, nt 941 to nt 1062 of SEQ ID No. 47, nt 940 to nt 1061 of SEQ ID No. 48, nt 941 to nt 1062 of SEQ ID No. 49, nt 940 to nt 1061 of SEQ ID No. 50, nt 940 to nt 1061 of SEQ ID No. 51, nt 940 to nt 1061 of SEQ ID No. 52, nt 941 to nt 1062 of SEQ ID No. 53, nt 941 to nt 1062 of SEQ ID No. 54, nt 940 to nt 1061 of SEQ ID No. 55, nt 940 to nt 1061 of SEQ ID No. 56, nt 940 to nt 1061 of SEQ ID No. 57, nt 940 to nt 1061 of SEQ ID No. 58, nt 940 to nt 1061 of SEQ ID No. 59, nt 941 to nt 1062 of SEQ ID No. 60, nt 941 to nt 1062 of SEQ ID No. 61, nt 940 to nt 1061 of SEQ ID No. 62, nt 940 to nt 1061 of SEQ ID No. 63, nt 941 to nt 1062 of SEQ ID No. 64, nt 940 to nt 1061 of SEQ ID No. 65, nt 940 to nt 1061 of SEQ ID No. 66, nt 941 to nt 1062 of SEQ ID No. 67, nt 941 to nt 1062 of SEQ ID No. 68, nt 940 to nt 1061 of SEQ ID No. 69, nt 940 to nt 1061 of SEQ ID No. 70, nt 941 to nt 1062 of SEQ ID No. 71, nt 940 to nt 1061 of SEQ ID No. 72, nt 941 to nt 1062 of SEQ ID No. 73, nt 941 to nt 1062 of SEQ ID No. 74, nt 940 to nt 1061 of SEQ ID No. 75, nt 940 to nt 1061 of SEQ ID No. 76, nt 940 to nt 1061 of SEQ ID No. 77, nt 940 to nt 1061 of SEQ ID No. 78, nt 941 to nt 1062 of SEQ ID No. 79, nt 941 to nt 1062 of SEQ ID No. 80, nt 940 to nt 1061 of SEQ ID No. 81, nt 940 to nt 1061 of SEQ ID No. 82, nt 940 to nt 1061 of SEQ ID No. 83, nt 940 to nt 1061 of SEQ ID No. 84, nt 941 to nt 1062 of SEQ ID No. 85, nt 941 to nt 1062 of SEQ ID No. 86, nt 941 to nt 1062 of SEQ ID No. 87, nt 941 to nt 1062 of SEQ ID No. 88, nt 941 to nt 1062 of SEQ ID No. 89, nt 940 to nt 1061 of SEQ ID No. 90, nt 940 to nt 1061 of SEQ ID No. 91, nt 940 to nt 1061 of SEQ ID No. 92, nt 941 to nt 1062 of SEQ ID No. 93, nt 940 to nt 1061 of SEQ ID No. 94, nt 941 to nt 1062 of SEQ ID No. 95, nt 941 to nt 1062 of SEQ ID No. 96, nt 941 to nt 1062 of SEQ ID No. 97, nt 941 to nt 1062 of SEQ ID No. 98, an allelic variant or a complementary sequence thereof.

23. The method of claim 1 further comprising determining the methylation status of at least one cytosine residue in the methylation relevant region of the LINE-1 repetitive sequence as in SEQ ID No. 220.

24. An in vitro method of prognosing melanoma within a clinico-pathological stage thereof and/or monitoring the response to a melanoma therapy in a subject and/or to identify a subject to be treated with a DNA hypomethylating agent comprising determining the methylation status of at least one cytosine residue in a methylation relevant region of:
a] at least one of the following genes: ALOX12B, CTAG2, FGF4, IGLL1, OCIAD2, PAGE5, S100A9, SLC6A11, SLC6A18, TUB, BATF, BGN, CRHR1, CSAG1, CTSK, DENND2D, FLJ33860, GJB5, GRM4, LY96, MAGEA1, MAGEA10, MGC35206, MLSTD1, SLC18A2, SORBS2, and TRIM40; and/or
b] the LINE-1 repetitive sequence as in SEQ ID No. 220.

25. The method of claim 1 wherein the melanoma is a cutaneous melanoma.

26. The method of claim 1 comprising the steps of:
(a) isolating DNA from a sample obtained from a subject;
(b) subjecting said DNA to methylation-sensitive sequence modification through incubation with suitable chemicals, or contacting said DNA with a reagent capable of selectively binding to methylated DNA;
(c) optionally amplifying said DNA as obtained in b), and
(d) determining the methylation status of said DNA.

27. A kit for performing an in vitro method of prognosing melanoma within a clinico-pathological stage thereof and/or monitoring the response to a melanoma therapy in a subject and/or to identify a subject to be treated with a DNA hypomethylating agent, comprising a chemical able to differentially modify DNA sequence depending on methylation status, or a reagent capable of selectively binding to methylated DNA, and probes, primers and/or aptamers specific for each of genes as defined in claim 1.

28. The kit of claim 27 in the form of an array.

29. The method of claim 1, wherein the melanoma is at stage III or IIIC.
